SEQUENCE LISTING (533 sequences)

SEQ ID NO: 1 - DNA, 1578 bp, synthetic construct
  source 1..1578: mol_type = other DNA
atggcacatatgaccaatgtgcgccgggtgtttaatgaattgattggcgacgatgaaagtgaaactcaggaagagtcgctgtcggaacagtggcgtgagctgtggcaggatgcgttgcaggaagatgacactacgccagtgctggcgcatcttagcgaggatgatcgcaaacaggtgctaacgctgattgccgatttccgcaaagagctggataagcgcaccatcgggccgcgaggacgtcaggtgctcgaccatctgatgccgcatctgctaagtgatgtctgtgcgcgtgaagacgctgccgttacgctgtcgcgcattaccgccttgctggtggggattgttacccgcaccacctatttagaattgctcagtgaattccccgcggcgcttaaacatttgatttctctgtgtgccgcgtcgccgatgattgccagccagctggcgcgttatccattattgctggatgaattgctcgatccaaacaccctttaccagccgacggcgaccgatgcctaccgcgatgagttgcgccagtatttgctgcgcgtgccggaagatgacgaagagcaacagcttgaggcgctgcgtcagttcaaacaggcgcagctgttacgcatcgccgcagcggatatcgccggtacgctaccggtgatgaaagtgagcgatcacttaacctggctggcggaagccatgatagatgccgtcgttcagcaggcgtgggttcaaatggttgcccgctacggtaagccgaatcacctgaacgaacgcgaagggcgtggttttgcggtggtcggctacggcaagctgggcggctgggagttaggctacagttccgatcttgaccttatcttcctccatgattgcccaatggatgcgatgactgacggtgagcgggaaatcgacgggcggcagttttatctgcgtctggcgcaacgcattatgcatctgttcagtacgcgtacctcttccggcattttgtatgaagtggatgctcgactgcgtccgtccggggcggcgggaatgctggtgacatccgcagaagcatttgccgattatcagaaaaacgaggcctggacgtgggaacatcaggcgctggtgcgtgcgcgtgtagtgtacggcgatccgcagctcaccgcgcactttgacgcagtgcgtcgcgagattatgacgctgccgcgtgaaggtaaaactctgcaaacggaagtgcgggaaatgcgcgagaaaatgcgcgctcatctcggcaataaacatcgcgatcgctttgatatcaaagctgatgaagggggaattaccgatatcgaatttattacccaatatctggtgttgcgctacgctcatgaaaaaccgaagttaacgcgctggtcagacaacgtgcgtattctggaactactggcgcaaaacgacattatggaagagcaggaagcgatggcgctgacccgtgcttacactacgcttcgcgatgaacttcatcatctggcattacaggaattgccgggccatgtgtcggaggattgcttcaccgcagagcgtgaactggtgcgggcaagctggcagaagtggctggtggaagaatga

SEQ ID NO: 2 - DNA, 1578 bp, synthetic construct
  source 1..1578: mol_type = other DNA
atggctcatatgaccaatgtgcgacgtgttttcaacgaactcatcggggacgacgaatcggaaacgcaggaagagagcctctctgaacagtggcgtgagctgtggcaggatgcactgcaagaggatgatacgaccccggtgcttgcccatctgagtgaagacgataggaagcaggttctgaccctgatcgcggacttccgaaaggaacttgataaacgcaccattggaccgcggggtcgtcaagtgctggatcatctgatgccccatcttctgtcggatgtgtgcgcacgagaggatgctgccgtcaccctctcgcgcattaccgcccttctggtcggtatcgtgacccgcactacgtacctcgagcttctctcggagttccccgcagccctcaagcatctgatctccttgtgcgcggcctcgccaatgatcgcgtcgcaattggcccgttacccgctgttgcttgatgagcttctggatccaaataccttgtaccagccaacggcgaccgatgcctacagggatgaactccgccagtatctccttcgggttcccgaagacgacgaagaacaacagctcgaagcgcttcggcaatttaagcaggcccaattgcttcgcatcgcggcagcggacattgctggcacccttccggtgatgaaagtttcggaccatctgacatggctggcggaggccatgattgacgccgtcgtccagcaagcgtgggtgcagatggtcgctcgttatggaaagcccaaccacctgaatgagcgcgagggccgcggattcgcggtggttggctatggcaagctgggtggttgggaattggggtattcctccgacctggatctgatcttcttacatgactgccctatggacgccatgacagatggcgagcgggagatcgacggtagacaattttaccttcgcctggcccaacgcataatgcatttattctcgacgcgcacgtcgagtggtatcctttacgaagtggacgcgagactgcggccttccggcgccgcaggcatgctggtgacctcggccgaagcgttcgccgattaccaaaaaaacgaagcctggacgtgggaacatcaagctcttgtccgcgcgcgcgtggtctatggagatccgcagctcaccgcccatttcgacgcggtgcggcgcgaaattatgacgctcccacgcgaaggcaagacgctgcagaccgaggtgcgcgaaatgcgggagaaaatgcgcgcccatttgggtaataagcaccgcgacaggttcgatatcaaggcggacgaaggcggaatcacggacatcgaatttattactcaatatttggtcctgcgatatgcacacgaaaagcccaaactaacacgctggtccgataacgtcaggatcttagagcttcttgctcagaacgatatcatggaagagcaagaggctatggcgcttacccgtgcctacacgacgctgcgggatgaacttcatcatcttgcgctgcaggaactgccgggtcatgtctcagaagattgctttaccgctgaacgcgaactggtccgggcaagctggcagaagtggctggtcgaagagtga

SEQ ID NO: 3 - DNA, 1578 bp, synthetic construct
  source 1..1578: mol_type = other DNA
atggcgcacatgacgaacgtcaggagagtcttcaacgagctcatcggggatgacgagtcggagacgcaagaggaaagcttaagcgagcaatggagggaattatggcaagacgcactccaagaggacgataccacccccgtcttagcccacttgtcggaagacgacaggaagcaagttttgaccttaatcgcggactttaggaaggaattagacaaaaggacgattggccccaggggcaggcaagtcttggatcacttaatgccccacttattgtccgacgtgtgcgccagggaggatgccgcagtgaccttaagcagaatcacggcgctcttagtcggcatcgtcacgaggacgacgtacctggagctcttgtcggagtttccggccgccttgaagcacctcatcagcttatgcgcggccagccccatgatcgcgtcgcaattagccagatacccgctgcttttagacgagctcttggacccgaatacgttgtatcaacccaccgccacagacgcgtatagggacgaactcagacaataccttttaagagtccccgaggacgatgaggaacagcaattggaagccttaaggcaatttaagcaagcccaattactgagaattgcagccgcagacattgcgggcaccttgcccgttatgaaggtttcggaccatctgacgtggttagccgaggcgatgatcgacgcggtggtccaacaagcctgggtccagatggtcgcgaggtatggcaaaccaaaccatttaaatgagagagagggcagaggcttcgcagtcgtgggatatgggaaattaggggggtgggaactggggtattccagtgacttggatttgatatttttgcacgactgtcccatggacgccatgacggatggcgaaagagagattgatggaagacaattctacttaaggttagcccagaggatcatgcacttattttccaccaggacgagcagtgggatcctctacgaggtcgacgccaggttaagacctagtggcgccgccggcatgttagtcaccagtgctgaggcgttcgcggactaccaaaagaatgaagcatggacctgggagcaccaagccttagttagggccagggtggtctatggagacccccaattgacggcccatttcgatgcggtcagaagggaaatcatgaccttacccagggagggcaagaccttacagaccgaggtcagagagatgagagaaaagatgagggcccacttggggaacaagcacagagacaggttcgacataaaggcggacgagggcggcatcacggacattgagttcatcactcagtacttagtcctcagatatgcccacgagaagcccaaactgaccagatggagtgataatgtcagaatcttagagttgttagctcagaatgatatcatggaggaacaagaggccatggccttaacgagggcctatacgaccttgagggacgagttgcaccacttagcgctgcaagagctccccgggcacgtcagcgaagactgttttacggccgaaagagagttagttagagcgtcttggcaaaaatggttagtcgaggagtga

SEQ ID NO: 4 - DNA, 1578 bp, synthetic construct
  source 1..1578: mol_type = other DNA
atggcccacatgactaacgtacgacgcgttttcaacgagcttataggtgacgacgaaagcgaaacccaggaggagtctctcagtgaacagtggcgcgaactctggcaggacgcccttcaggaagacgacacgactcctgttcttgcacatctctcagaggacgatcgtaagcaggtcctcactctcatagctgacttccgtaaggaactcgacaagcgtactataggaccacgcgggcgccaggtacttgaccatcttatgcctcatctcctctcggacgtatgtgctcgcgaagacgcggcggtcacactctcacgtataactgcacttctcgtaggtatagtgactagaacaacttacctcgagcttctaagcgagttccctgctgctctcaagcaccttatatcgctctgcgctgcatctccaatgatagcttctcagctcgctcgctaccccctcctcctcgatgaacttctagaccccaacactctctaccagcctactgcaacggacgcataccgtgatgaactacggcagtacctcctccgggttcctgaagacgatgaagaacaacagctcgaagctctccgccagttcaaacaggctcagctcctccggatagcggcggccgacatagcagggacactcccagtcatgaaagtctcagaccacctcacatggctcgctgaggcaatgattgacgctgttgtgcaacaggcatgggtgcagatggtggctcgttacggaaagcccaaccatctcaacgaacgggaaggacgggggttcgccgttgttgggtacggtaagctcggaggatgggaactcggttactcgtcggacctcgacctcattttccttcatgactgcccgatggacgctatgaccgatggagaacgtgaaatagacggccgccagttctatctccgcctcgcacagcgaataatgcacctcttctcgacacgcacttcgtcgggtatactatatgaagttgacgcgcgcctccggccctcgggtgctgcagggatgctcgttacgtcggcggaagccttcgctgactatcagaagaatgaagcgtggacttgggaacatcaggcactcgtccgcgctcgcgtcgtttacggggacccacagcttactgctcacttcgacgccgttcgccgagaaataatgactcttccacgcgaagggaagacgcttcagactgaggttcgcgaaatgcgggaaaagatgcgtgcgcaccttggtaacaagcaccgggaccgattcgatattaaggccgatgaaggtgggataacagatatagaattcataacgcagtacctcgttcttcggtacgcgcacgaaaagcctaagctcacacggtggtcggacaacgttcggatactcgaacttcttgcccagaacgatataatggaagaacaggaagctatggctctcactcgcgcgtacaccactctccgtgacgagctccatcatctcgcccttcaggaacttcctggtcacgtttctgaagactgcttcacagcggaacgcgaactcgtccgcgcctcgtggcagaagtggcttgtagaggaatga

SEQ ID NO: 5 - DNA, 1578 bp, synthetic construct
  source 1..1578: mol_type = other DNA
atggctcacatgacaaacgttcgtcgtgtattcaacgaactaatcggagacgacgaatcagaaacacaggaagaatcactttcagaacagtggcgagaactttggcaggacgctctacaggaagatgatacaacaccggtactcgctcacttaagtgaagacgaccgaaagcaggtacttacacttatcgcagacttccgaaaggaacttgacaagcgaacaattggtcctcgtggtcgacaggttctagatcatctcatgccacaccttctttcagacgtttgcgcacgagaagacgcagctgtaactctttctcggatcacagctctacttgttggaatcgtaacacgtactacataccttgaactactttcagaattcccagcagcactaaagcacctaatctcactttgcgcagcttcacctatgatcgcatcacaacttgcacggtatcctcttctacttgacgagctacttgaccctaacacactatatcagccaacagctactgacgcttaccgagacgagcttcgtcagtatctacttcgtgtaccagaggacgacgaggagcagcagctagaagcacttagacagttcaagcaggcacagcttcttcgtattgctgctgctgacatcgctggaactcttcctgtaatgaaggtaagtgaccaccttacttggcttgcagaagctatgatcgacgcagtagtacagcaggcttgggtacaaatggtagcacgatatgggaaacctaaccaccttaacgaacgtgaaggtcgcggattcgctgtagtaggttacggaaagcttggtggttgggaacttggatactcatcagacttagacctaattttcctacacgattgccctatggacgcaatgacagacggggaacgcgagatcgatggtcgtcagttctaccttcgacttgctcagcgtatcatgcatcttttctcaactcgaacatcatcaggaatcctttacgaagtagacgcacgtcttcgcccatcaggagcagctggtatgcttgtaacttcagccgaagctttcgcagattaccagaagaacgaagcttggacatgggaacaccaggctcttgtacgagcaagagttgtatatggtgaccctcagttaacagcacacttcgacgctgtacggcgtgagatcatgacactccctcgagagggaaagacactccagacagaagtacgtgaaatgcgtgaaaagatgcgagcacacctaggaaacaagcatcgtgaccgtttcgacatcaaggcagacgaaggaggtatcactgacatcgagttcatcacacagtaccttgtactacgttacgcacacgaaaagccaaagcttactcgttggtccgacaacgtacgcatccttgaactcctcgcacagaacgacatcatggaagaacaagaagcaatggcacttacacgagcatatacaacactacgagacgaactacaccaccttgctctccaggagctaccaggacacgtatcagaggactgcttcactgctgaacgggagcttgtacgtgcttcatggcagaagtggctcgttgaagaatga

SEQ ID NO: 6 - DNA, 1020 bp, synthetic construct
  source 1..1020: mol_type = other DNA
atggacgaagagcaacagcttgaggcgctgcgtcagttcaaacaggcgcagctgttacgcatcgccgcagcggatatcgccggtacgctaccggtgatgaaagtgagcgatcacttaacctggctggcggaagccatgatagatgccgtcgttcagcaggcgtgggttcaaatggttgcccgctacggtaagccgaatcacctgaacgaacgcgaagggcgtggttttgcggtggtcggctacggcaagctgggcggctgggagttaggctacagttccgatcttgaccttatcttcctccatgattgcccaatggatgcgatgactgacggtgagcgggaaatcgacgggcggcagttttatctgcgtctggcgcaacgcattatgcatctgttcagtacgcgtacctcttccggcattttgtatgaagtggatgctcgactgcgtccgtccggggcggcgggaatgctggtgacatccgcagaagcatttgccgattatcagaaaaacgaggcctggacgtgggaacatcaggcgctggtgcgtgcgcgtgtagtgtacggcgatccgcagctcaccgcgcactttgacgcagtgcgtcgcgagattatgacgctgccgcgtgaaggtaaaactctgcaaacggaagtgcgggaaatgcgcgagaaaatgcgcgctcatctcggcaataaacatcgcgatcgctttgatatcaaagctgatgaagggggaattaccgatatcgaatttattacccaatatctggtgttgcgctacgctcatgaaaaaccgaagttaacgcgctggtcagacaacgtgcgtattctggaactactggcgcaaaacgacattatggaagagcaggaagcgatggcgctgacccgtgcttacactacgcttcgcgatgaacttcatcatctggcattacaggaattgccgggccatgtgtcggaggattgcttcaccgcagagcgtgaactggtgcgggcaagctggcagaagtggctggtggaagaatga

SEQ ID NO: 7 - DNA, 1671 bp, synthetic construct
  source 1..1671: mol_type = other DNA
atgctcggccgggtggaggaccgctacgccgagctgttcgaggaggcgccgtcgctgtccggccccggcaacctcgtcttcaccggcaccgacgacgaccccggcacggtgaagacgctggccggcatgggctaccgcgaccccagccgggtcatcgccgtggtctccacctggcaccgcgggcgctaccgctccacccgctcgggccgcgcgcgggagctgctgaccgagctggtgccggccatgctgaacgagctggcgaagacccccgccccggacgacgcgctggttaagttcgacagcttcctggagcggcttccggcgggggtcgggctgttctcgctgttcatcgccaacccctggctgctcgccctggtcgcggagatcatgggcacggcgccgcagctggccgagacgttgtcgcgcaacccgtcgctgctcgacgccgtgctgtcgcccgacttcttcgacccgctgcccgacgcggcggggctgacgccggagtaccagcgcttcatcgccggggcgcacaacttcgaggatgtgctgaccctgtcgcggcgctggaccaacgaccagcgcttccgcgccggggcgcacatcctgcgcggcatcaccgacggcgaccgctgcggccccttcctcgccgatctggccgacgtggtggtgccggagctggccgcccgcgtggaggaggagttcgccgcccgccacggccgcatccccggcggcgcctgggtggtggtggcgatgggcaagctaggcagccggcagctcaccatcacctccgacatcgacctgatcgtggtctacgaggtgccgccgggcacccgccagtcggacggagccaagccgctggcccccaacgagtattacatcaagctgacgcagcgcctgaccaacgccatcaccgccccgatggccgacgggcggctgtacgaggtggacatgcggctgcgcccgtcgggcaacgccggtccgctcgccaccgcgctggacgccttcaccgcctatcaggccaaggatgcctggacgtgggagcacatggccctgacccgcgcccgcgtcatcggcagtgattccggcggcggcgatccggcgctgggtcataaggtcgagtcggcgatccgcggtgtgctgaccggcccgcgcgacccggccaaggtgctgcgggacgtcgccgacatgcgccgccgcatcgacaaggagttcggcaccaccaacccgtggaacgtcaaatacgcccgcggcggcctgatcgacatcgagttcacggcccagtatctccagctccgtcacggccacgcgcatccggacatcctgtccatcgccaccagccgcgccctgctcaacgccgctgcggccgggctgctggcgccggaggtggcggaggagctggtggcgacgctgaagctgtggcggcgggtgcagggcttcctgcgcctgaccaccgacggcgtgctcgatccgcggcaggtttcgcccaccctgcgggagggcctctcgcgcgccgccttcccggacgaggagccggcggttgacttcgccgcgctcgacagcagaatccgggacatcgccgcccgcgcccaccgccatttcgtggcgctggtcgaggagccggcgtcaaggctgcctcccccagagaccaacgaagaagccaaactcccatga

SEQ ID NO: 8 - DNA, 1575 bp, synthetic construct
  source 1..1575: mol_type = other DNA
atgcccggcacggtgaagacgctggccggcatgggctaccgcgaccccagccgggtcatcgccgtggtctccacctggcaccgcgggcgctaccgctccacccgctcgggccgcgcgcgggagctgctgaccgagctggtgccggccatgctgaacgagctggcgaagacccccgccccggacgacgcgctggttaagttcgacagcttcctggagcggcttccggcgggggtcgggctgttctcgctgttcatcgccaacccctggctgctcgccctggtcgcggagatcatgggcacggcgccgcagctggccgagacgttgtcgcgcaacccgtcgctgctcgacgccgtgctgtcgcccgacttcttcgacccgctgcccgacgcggcggggctgacgccggagtaccagcgcttcatcgccggggcgcacaacttcgaggatgtgctgaccctgtcgcggcgctggaccaacgaccagcgcttccgcgccggggcgcacatcctgcgcggcatcaccgacggcgaccgctgcggccccttcctcgccgatctggccgacgtggtggtgccggagctggccgcccgcgtggaggaggagttcgccgcccgccacggccgcatccccggcggcgcctgggtggtggtggcgatgggcaagctaggcagccggcagctcaccatcacctccgacatcgacctgatcgtggtctacgaggtgccgccgggcacccgccagtcggacggagccaagccgctggcccccaacgagtattacatcaagctgacgcagcgcctgaccaacgccatcaccgccccgatggccgacgggcggctgtacgaggtggacatgcggctgcgcccgtcgggcaacgccggtccgctcgccaccgcgctggacgccttcaccgcctatcaggccaaggatgcctggacgtgggagcacatggccctgacccgcgcccgcgtcatcggcagtgattccggcggcggcgatccggcgctgggtcataaggtcgagtcggcgatccgcggtgtgctgaccggcccgcgcgacccggccaaggtgctgcgggacgtcgccgacatgcgccgccgcatcgacaaggagttcggcaccaccaacccgtggaacgtcaaatacgcccgcggcggcctgatcgacatcgagttcacggcccagtatctccagctccgtcacggccacgcgcatccggacatcctgtccatcgccaccagccgcgccctgctcaacgccgctgcggccgggctgctggcgccggaggtggcggaggagctggtggcgacgctgaagctgtggcggcgggtgcagggcttcctgcgcctgaccaccgacggcgtgctcgatccgcggcaggtttcgcccaccctgcgggagggcctctcgcgcgccgccttcccggacgaggagccggcggttgacttcgccgcgctcgacagcagaatccgggacatcgccgcccgcgcccaccgccatttcgtggcgctggtcgaggagccggcgtcaaggctgcctcccccagagaccaacgaagaagccaaactcccatga

SEQ ID NO: 9 - DNA, 1500 bp, synthetic construct
  source 1..1500: mol_type = other DNA
atgcaccgcgggcgctaccgctccacccgctcgggccgcgcgcgggagctgctgaccgagctggtgccggccatgctgaacgagctggcgaagacccccgccccggacgacgcgctggttaagttcgacagcttcctggagcggcttccggcgggggtcgggctgttctcgctgttcatcgccaacccctggctgctcgccctggtcgcggagatcatgggcacggcgccgcagctggccgagacgttgtcgcgcaacccgtcgctgctcgacgccgtgctgtcgcccgacttcttcgacccgctgcccgacgcggcggggctgacgccggagtaccagcgcttcatcgccggggcgcacaacttcgaggatgtgctgaccctgtcgcggcgctggaccaacgaccagcgcttccgcgccggggcgcacatcctgcgcggcatcaccgacggcgaccgctgcggccccttcctcgccgatctggccgacgtggtggtgccggagctggccgcccgcgtggaggaggagttcgccgcccgccacggccgcatccccggcggcgcctgggtggtggtggcgatgggcaagctaggcagccggcagctcaccatcacctccgacatcgacctgatcgtggtctacgaggtgccgccgggcacccgccagtcggacggagccaagccgctggcccccaacgagtattacatcaagctgacgcagcgcctgaccaacgccatcaccgccccgatggccgatgggcggctgtacgaggtggacatgcggctgcgcccgtcgggcaacgccggtccgctcgccaccgcgctggacgccttcaccgcctatcaggccaaggatgcctggacgtgggagcacatggccctgacccgcgcccgcgtcatcggcagtgattccggcggcggcgatccggcgctgggtcataaggtcgagtcggcgatccgcggtgtgctgaccggcccgcgcgacccggccaaggtgctgcgggacgtcgccgacatgcgccgccgcatcgacaaggagttcggcaccaccaacccgtggaacgtcaaatacgcccgcggcggcctgatcgacatcgagttcacggcccagtatctccagctccgtcacggccacgcgcatccggacatcctgtccatcgccaccagccgcgccctgctcaacgccgctgcggccgggctgctggcgccggaggtggcggaggagctggtggcgacgctgaagctgtggcggcgggtgcagggcttcctgcgcctgaccaccgacggcgtgctcgatccgcggcaggtttcgcccaccctgcgggagggcctctcgcgcgccgccttcccggacgaggagccggcggttgacttcgccgcgctcgacagcagaatccgggacatcgccgcccgcgcccaccgccatttcgtggcgctggtcgaggagccggcgtcaaggctgcctcccccagagaccaacgaagaagccaaactcccatga

SEQ ID NO: 10 - DNA, 1428 bp, synthetic construct
  source 1..1428: mol_type = other DNA
atgctgaacgagctggcgaagacccccgccccggacgacgcgctggttaagttcgacagcttcctggagcggcttccggcgggggtcgggctgttctcgctgttcatcgccaacccctggctgctcgccctggtcgcggagatcatgggcacggcgccgcagctggccgagacgttgtcgcgcaacccgtcgctgctcgacgccgtgctgtcgcccgacttcttcgacccgctgcccgacgcggcggggctgacgccggagtaccagcgcttcatcgccggggcgcacaacttcgaggatgtgctgaccctgtcgcggcgctggaccaacgaccagcgcttccgcgccggggcgcacatcctgcgcggcatcaccgacggcgaccgctgcggccccttcctcgccgatctggccgacgtggtggtgccggagctggccgcccgcgtggaggaggagttcgccgcccgccacggccgcatccccggcggcgcctgggtggtggtggcgatgggcaagctaggcagccggcagctcaccatcacctccgacatcgacctgatcgtggtctacgaggtgccgccgggcacccgccagtcggacggagccaagccgctggcccccaacgagtattacatcaagctgacgcagcgcctgaccaacgccatcaccgccccgatggccgacgggcggctgtacgaggtggacatgcggctgcgcccgtcgggcaacgccggtccgctcgccaccgcgctggacgccttcaccgcctatcaggccaaggatgcctggacgtgggagcacatggccctgacccgcgcccgcgtcatcggcagtgattccggcggcggcgatccggcgctgggtcataaggtcgagtcggcgatccgcggtgtgctgaccggcccgcgcgacccggccaaggtgctgcgggacgtcgccgacatgcgccgccgcatcgacaaggagttcggcaccaccaacccgtggaacgtcaaatacgcccgcggcggcctgatcgacatcgagttcacggcccagtatctccagctccgtcacggccacgcgcatccggacatcctgtccatcgccaccagccgcgccctgctcaacgccgctgcggccgggctgctggcgccggaggtggcggaggagctggtggcgacgctgaagctgtggcggcgggtgcagggcttcctgcgcctgaccaccgacggcgtgctcgatccgcggcaggtttcgcccaccctgcgggagggcctctcgcgcgccgccttcccggacgaggagccggcggttgacttcgccgcgctcgacagcagaatccgggacatcgccgcccgcgcccaccgccatttcgtggcgctggtcgaggagccggcgtcaaggctgcctcccccagagaccaacgaagaagccaaactcccatga

SEQ ID NO: 11 - DNA, 1143 bp, synthetic construct
  source 1..1143: mol_type = other DNA
atgaacttcgaggatgtgctgaccctgtcgcggcgctggaccaacgaccagcgcttccgcgccggggcgcacatcctgcgcggcatcaccgacggcgaccgctgcggccccttcctcgccgatctggccgacgtggtggtgccggagctggccgcccgcgtggaggaggagttcgccgcccgccacggccgcatccccggcggcgcctgggtggtggtggcgatgggcaagctaggcagccggcagctcaccatcacctccgacatcgacctgatcgtggtctacgaggtgccgccgggcacccgccagtcggacggagccaagccgctggcccccaacgagtattacatcaagctgacgcagcgcctgaccaacgccatcaccgccccgatggccgacgggcggctgtacgaggtggacatgcggctgcgcccgtcgggcaacgccggtccgctcgccaccgcgctggacgccttcaccgcctatcaggccaaggatgcctggacgtgggagcacatggccctgacccgcgcccgcgtcatcggcagtgattccggcggcggcgatccggcgctgggtcataaggtcgagtcggcgatccgcggtgtgctgaccggcccgcgcgacccggccaaggtgctgcgggacgtcgccgacatgcgccgccgcatcgacaaggagttcggcaccaccaacccgtggaacgtcaaatacgcccgcggcggcctgatcgacatcgagttcacggcccagtatctccagctccgtcacggccacgcgcatccggacatcctgtccatcgccaccagccgcgccctgctcaacgccgctgcggccgggctgctggcgccggaggtggcggaggagctggtggcgacgctgaagctgtggcggcgggtgcagggcttcctgcgcctgaccaccgacggcgtgctcgatccgcggcaggtttcgcccaccctgcgggagggcctctcgcgcgccgccttcccggacgaggagccggcggttgacttcgccgcgctcgacagcagaatccgggacatcgccgcccgcgcccaccgccatttcgtggcgctggtcgaggagccggcgtcaaggctgcctcccccagagaccaacgaagaagccaaactcccatga

SEQ ID NO: 12 - DNA, 1500 bp, synthetic construct
  source 1..1500: mol_type = other DNA
atgcatagaggcagatatagaagtacgaggagcggaagggccagagaattattaacggaattagtccccgcaatgttaaatgaattagccaaaacgcctgcgcccgatgatgccttagtgaaatttgattcgtttttagaaagattgcccgccggcgtgggcttatttagcttatttattgcgaatccatggttattggcgttagtggccgaaattatgggtaccgccccccaattagcggaaactctaagtaggaatcctagtttattagatgctgtcttaagcccggatttttttgatcccttaccggatgccgccggcttaacccccgaatatcaaaggtttattgcgggcgcccataattttgaagacgttttaactttaagcagaaggtggacgaatgatcaaagatttagggcgggcgcccatattttaagaggtattacggatggagataggtgtgggccgtttttggctgacttagcggatgtcgttgtcccagaattagcagcgagggtcgaagaagaatttgcggcgagacatggtagaattccgggaggtgcgtgggtcgtcgtcgccatgggaaaattgggttcgagacaattgacgattacgagtgatattgatttaattgtcgtgtatgaagttccccctggtacgaggcaaagcgatggcgctaaacccttagcgccgaatgaatactatattaaattaacccaaaggttaacgaatgcgattacggcgcccatggcagacggcagattatatgaagtcgatatgagattaaggcccagcgggaatgcgggccccttggcgacggccttagatgcgtttacggcgtaccaagcgaaagacgcttggacctgggaacatatggcgttaacgagagctagagtgataggttcggacagtggtgggggtgaccccgccttaggccacaaagtggaaagcgccattaggggcgtcttaacggggcccagggatcccgcgaaagtcttaagagatgtggcggatatgaggaggaggattgataaagaatttgggacgacgaatccctggaatgtaaagtatgcgaggggggggttaattgatatagaatttaccgcgcaatacttgcaattgaggcatgggcatgcccaccctgatattttaagtattgcgacgtcgagagcgttattgaatgcggccgccgcgggattattagcccccgaagtcgccgaagaattagtcgccaccttaaaattatggagaagagtccaaggatttttaagattaacgacagatggggtcttagaccctagacaagtgagcccgacgttaagagaagggttgagcagggcggcgtttcccgatgaagaacccgccgtcgattttgcggccttggattcgcggattagagatattgcggcgagagcgcataggcactttgtcgccttagtggaagaacccgccagccgcttaccgccgccggaaacaaatgaggaggcgaagttgccctga

SEQ ID NO: 13 - DNA, 1413 bp, synthetic construct
  source 1..1413: mol_type = other DNA
atgcgtgcgctgcgcacggacgcggcgcgtgagcatctcgcccgcatcgtggcccttctcatcgagcaactgtcccatggcggcgacccggacggcgcactcaatgccatggacagcttcctgcgcgacctgcccggtccgcacctgctggcggcgctggagcacaatccggacctcgtgcgcctcatcgccaccatcgtcagcgccgccccgcgcctcggcgagacgctggcccggcgcccctcgctcgccgatgcgctgctcgatcccgccttcttcgacgtgctgcccgacgagccggccctcgccgcccatctcgaagccttgctcgatgcggcgccgaatgaggaagaacagttcgaccgcgcccgccgcttccggcaggaacagcacgttctgatcggcgtgcgcatcgtctccggcacgctgccggcggcgcgggcgggcgaggcctatgcccgcgtggcggaggtcatcatccgcgccctgcacgcccgcgtctgggcgcgcttcgtggaggcgcacggcagcattcccggcgcggatacggcagtgctctccatgggcaagctcggcagccgcgagatgaccgccggctccgacctcgatctcatcgtgctctacgatttcgatccggcctcggacggcacgtcggatggcccgcgcccgctggtcggagcgcagtatttcgcccgcttcacccagcgcctcgtcaccgcgctgaccagcctcaccaatgccggcaagctctatgacgtggacctgcgcctgcgcccgtccggccggtcaggcccggtggcgacgcgcatcggctccttcgagaattaccagcgcaccgaggcctggacctgggagcacatggcgctgacccgggcgcggatcatctccgccagccccgcctttgcccggcgggtggagcaggtgatcctgcaggtgctcgcccatccgcgtgatccccgccgcatcgccggcgacattctcgacatgcgccgggccatcgcggcggagaagggcgaaagcgaccgctggaatctcaagcacgcggccggcggacaggtggacgcggagttcctcgcccagttcctcgtgctcatccatgccgaacggcatccggagatcgtcgataccgcgacggcgcgcatcctcaccgtggcgggctggcttcagctcctgagtcccgaggattgccagaccctctccaacgcctgccggctctatcaggatctcacgcaggtgctccgcctcgccatcgaccggccattcgtgccggcgcaggcgagcccggcgctgaaagcgctgctggcgcgggcgggcgagatgccggacttctcctccctcgacgcccatctcaccgacacggaagcgcgcgtgcgggccatcttcgagcgcattctggaagccgcttccgcctga

SEQ ID NO: 14 - DNA, 1383 bp, synthetic construct
  source 1..1383: mol_type = other DNA
atgcatctcgcccgcatcgtggcccttctcatcgagcaactgtcccatggcggcgacccggacggcgcactcaatgccatggacagcttcctgcgcgacctgcccggtccgcacctgctggcggcgctggagcacaatccggacctcgtgcgcctcatcgccaccatcgtcagcgccgccccgcgcctcggcgagacgctggcccggcgcccctcgctcgccgatgcgctgctcgatcccgccttcttcgacgtgctgcccgacgagccggccctcgccgcccatctcgaagccttgctcgatgcggcgccgaatgaggaagaacagttcgaccgcgcccgccgcttccggcaggaacagcacgttctgatcggcgtgcgcatcgtctccggcacgctgccggcggcgcgggcgggcgaggcctatgcccgcgtggcggaggtcatcatccgcgccctgcacgcccgcgtctgggcgcgcttcgtggaggcgcacggcagcattcccggcgcggatacggcagtgctctccatgggcaagctcggcagccgcgagatgaccgccggctccgacctcgatctcatcgtgctctacgatttcgatccggcctcggacggcacgtcggatggcccgcgcccgctggtcggagcgcagtatttcgcccgcttcacccagcgcctcgtcaccgcgctgaccagcctcaccaatgccggcaagctctatgacgtggacctgcgcctgcgcccgtccggccggtcaggcccggtggcgacgcgcatcggctccttcgagaattaccagcgcaccgaggcctggacctgggagcacatggcgctgacccgggcgcggatcatctccgccagccccgcctttgcccggcgggtggagcaggtgatcctgcaggtgctcgcccatccgcgtgatccccgccgcatcgccggcgacattctcgacatgcgccgggccatcgcggcggagaagggcgaaagcgaccgctggaatctcaagcacgcggccggcggacaggtggacgcggagttcctcgcccagttcctcgtgctcatccatgccgaacggcatccggagatcgtcgataccgcgacggcgcgcatcctcaccgtggcgggctggcttcagctcctgagtcccgaggattgccagaccctctccaacgcctgccggctctatcaggatctcacgcaggtgctccgcctcgccatcgaccggccattcgtgccggcgcaggcgagcccggcgctgaaagcgctgctggcgcgggcgggcgagatgccggacttctcctccctcgacgcccatctcaccgacacggaagcgcgcgtgcgggccatcttcgagcgcattctggaagccgcttccgcctga

SEQ ID NO: 15 - DNA, 1353 bp, synthetic construct
  source 1..1353: mol_type = other DNA
atggagcaactgtcccatggcggcgacccggacggcgcactcaatgccatggacagcttcctgcgcgacctgcccggtccgcacctgctggcggcgctggagcacaatccggacctcgtgcgcctcatcgccaccatcgtcagcgccgccccgcgcctcggcgagacgctggcccggcgcccctcgctcgccgatgcgctgctcgatcccgccttcttcgacgtgctgcccgacgagccggccctcgccgcccatctcgaagccttgctcgatgcggcgccgaatgaggaagaacagttcgaccgcgcccgccgcttccggcaggaacagcacgttctgatcggcgtgcgcatcgtctccggcacgctgccggcggcgcgggcgggcgaggcctatgcccgcgtggcggaggtcatcatccgcgccctgcacgcccgcgtctgggcgcgcttcgtggaggcgcacggcagcattcccggcgcggatacggcagtgctctccatgggcaagctcggcagccgcgagatgaccgccggctccgacctcgatctcatcgtgctctacgatttcgatccggcctcggacggcacgtcggatggcccgcgcccgctggtcggagcgcagtatttcgcccgcttcacccagcgcctcgtcaccgcgctgaccagcctcaccaatgccggcaagctctatgacgtggacctgcgcctgcgcccgtccggccggtcaggcccggtggcgacgcgcatcggctccttcgagaattaccagcgcaccgaggcctggacctgggagcacatggcgctgacccgggcgcggatcatctccgccagccccgcctttgcccggcgggtggagcaggtgatcctgcaggtgctcgcccatccgcgtgatccccgccgcatcgccggcgacattctcgacatgcgccgggccatcgcggcggagaagggcgaaagcgaccgctggaatctcaagcacgcggccggcggacaggtggacgcggagttcctcgcccagttcctcgtgctcatccatgccgaacggcatccggagatcgtcgataccgcgacggcgcgcatcctcaccgtggcgggctggcttcagctcctgagtcccgaggattgccagaccctctccaacgcctgccggctctatcaggatctcacgcaggtgctccgcctcgccatcgaccggccattcgtgccggcgcaggcgagcccggcgctgaaagcgctgctggcgcgggcgggcgagatgccggacttctcctccctcgacgcccatctcaccgacacggaagcgcgcgtgcgggccatcttcgagcgcattctggaagccgcttccgcctga

SEQ ID NO: 16 - protein, 525 aa, synthetic construct
MAHMTNVRRVFNELIGDDESETQEESLSEQWRELWQDALQEDDTTPVLAHLSEDDRKQVLTLIADFRKELDKRTIGPRGRQVLDHLMPHLLSDVCAREDAAVTLSRITALLVGIVTRTTYLELLSEFPAALKHLISLCAASPMIASQLARYPLLLDELLDPNTLYQPTATDAYRDELRQYLLRVPEDDEEQQLEALRQFKQAQLLRIAAADIAGTLPVMKVSDHLTWLAEAMIDAVVQQAWVQMVARYGKPNHLNEREGRGFAVVGYGKLGGWELGYSSDLDLIFLHDCPMDAMTDGEREIDGRQFYLRLAQRIMHLFSTRTSSGILYEVDARLRPSGAAGMLVTSAEAFADYQKNEAWTWEHQALVRARVVYGDPQLTAHFDAVRREIMTLPREGKTLQTEVREMREKMRAHLGNKHRDRFDIKADEGGITDIEFITQYLVLRYAHEKPKLTRWSDNVRILELLAQNDIMEEQEAMALTRAYTTLRDELHHLALQELPGHVSEDCFTAERELVRASWQKWLVEE

SEQ ID NO: 17 - protein, 339 aa, synthetic construct
MDEEQQLEALRQFKQAQLLRIAAADIAGTLPVMKVSDHLTWLAEAMIDAVVQQAWVQMVARYGKPNHLNEREGRGFAVVGYGKLGGWELGYSSDLDLIFLHDCPMDAMTDGEREIDGRQFYLRLAQRIMHLFSTRTSSGILYEVDARLRPSGAAGMLVTSAEAFADYQKNEAWTWEHQALVRARVVYGDPQLTAHFDAVRREIMTLPREGKTLQTEVREMREKMRAHLGNKHRDRFDIKADEGGITDIEFITQYLVLRYAHEKPKLTRWSDNVRILELLAQNDIMEEQEAMALTRAYTTLRDELHHLALQELPGHVSEDCFTAERELVRASWQKWLVEE

SEQ ID NO: 18 - protein, 556 aa, synthetic construct
MLGRVEDRYAELFEEAPSLSGPGNLVFTGTDDDPGTVKTLAGMGYRDPSRVIAVVSTWHRGRYRSTRSGRARELLTELVPAMLNELAKTPAPDDALVKFDSFLERLPAGVGLFSLFIANPWLLALVAEIMGTAPQLAETLSRNPSLLDAVLSPDFFDPLPDAAGLTPEYQRFIAGAHNFEDVLTLSRRWTNDQRFRAGAHILRGITDGDRCGPFLADLADVVVPELAARVEEEFAARHGRIPGGAWVVVAMGKLGSRQLTITSDIDLIVVYEVPPGTRQSDGAKPLAPNEYYIKLTQRLTNAITAPMADGRLYEVDMRLRPSGNAGPLATALDAFTAYQAKDAWTWEHMALTRARVIGSDSGGGDPALGHKVESAIRGVLTGPRDPAKVLRDVADMRRRIDKEFGTTNPWNVKYARGGLIDIEFTAQYLQLRHGHAHPDILSIATSRALLNAAAAGLLAPEVAEELVATLKLWRRVQGFLRLTTDGVLDPRQVSPTLREGLSRAAFPDEEPAVDFAALDSRIRDIAARAHRHFVALVEEPASRLPPPETNEEAKLP

SEQ ID NO: 19 - protein, 524 aa, synthetic construct
MPGTVKTLAGMGYRDPSRVIAVVSTWHRGRYRSTRSGRARELLTELVPAMLNELAKTPAPDDALVKFDSFLERLPAGVGLFSLFIANPWLLALVAEIMGTAPQLAETLSRNPSLLDAVLSPDFFDPLPDAAGLTPEYQRFIAGAHNFEDVLTLSRRWTNDQRFRAGAHILRGITDGDRCGPFLADLADVVVPELAARVEEEFAARHGRIPGGAWVVVAMGKLGSRQLTITSDIDLIVVYEVPPGTRQSDGAKPLAPNEYYIKLTQRLTNAITAPMADGRLYEVDMRLRPSGNAGPLATALDAFTAYQAKDAWTWEHMALTRARVIGSDSGGGDPALGHKVESAIRGVLTGPRDPAKVLRDVADMRRRIDKEFGTTNPWNVKYARGGLIDIEFTAQYLQLRHGHAHPDILSIATSRALLNAAAAGLLAPEVAEELVATLKLWRRVQGFLRLTTDGVLDPRQVSPTLREGLSRAAFPDEEPAVDFAALDSRIRDIAARAHRHFVALVEEPASRLPPPETNEEAKLP

SEQ ID NO: 20 - protein, 499 aa, synthetic construct
MHRGRYRSTRSGRARELLTELVPAMLNELAKTPAPDDALVKFDSFLERLPAGVGLFSLFIANPWLLALVAEIMGTAPQLAETLSRNPSLLDAVLSPDFFDPLPDAAGLTPEYQRFIAGAHNFEDVLTLSRRWTNDQRFRAGAHILRGITDGDRCGPFLADLADVVVPELAARVEEEFAARHGRIPGGAWVVVAMGKLGSRQLTITSDIDLIVVYEVPPGTRQSDGAKPLAPNEYYIKLTQRLTNAITAPMADGRLYEVDMRLRPSGNAGPLATALDAFTAYQAKDAWTWEHMALTRARVIGSDSGGGDPALGHKVESAIRGVLTGPRDPAKVLRDVADMRRRIDKEFGTTNPWNVKYARGGLIDIEFTAQYLQLRHGHAHPDILSIATSRALLNAAAAGLLAPEVAEELVATLKLWRRVQGFLRLTTDGVLDPRQVSPTLREGLSRAAFPDEEPAVDFAALDSRIRDIAARAHRHFVALVEEPASRLPPPETNEEAKLP

SEQ ID NO: 21 - protein, 475 aa, synthetic construct
MLNELAKTPAPDDALVKFDSFLERLPAGVGLFSLFIANPWLLALVAEIMGTAPQLAETLSRNPSLLDAVLSPDFFDPLPDAAGLTPEYQRFIAGAHNFEDVLTLSRRWTNDQRFRAGAHILRGITDGDRCGPFLADLADVVVPELAARVEEEFAARHGRIPGGAWVVVAMGKLGSRQLTITSDIDLIVVYEVPPGTRQSDGAKPLAPNEYYIKLTQRLTNAITAPMADGRLYEVDMRLRPSGNAGPLATALDAFTAYQAKDAWTWEHMALTRARVIGSDSGGGDPALGHKVESAIRGVLTGPRDPAKVLRDVADMRRRIDKEFGTTNPWNVKYARGGLIDIEFTAQYLQLRHGHAHPDILSIATSRALLNAAAAGLLAPEVAEELVATLKLWRRVQGFLRLTTDGVLDPRQVSPTLREGLSRAAFPDEEPAVDFAALDSRIRDIAARAHRHFVALVEEPASRLPPPETNEEAKLP

SEQ ID NO: 22 - protein, 380 aa, synthetic construct
MNFEDVLTLSRRWTNDQRFRAGAHILRGITDGDRCGPFLADLADVVVPELAARVEEEFAARHGRIPGGAWVVVAMGKLGSRQLTITSDIDLIVVYEVPPGTRQSDGAKPLAPNEYYIKLTQRLTNAITAPMADGRLYEVDMRLRPSGNAGPLATALDAFTAYQAKDAWTWEHMALTRARVIGSDSGGGDPALGHKVESAIRGVLTGPRDPAKVLRDVADMRRRIDKEFGTTNPWNVKYARGGLIDIEFTAQYLQLRHGHAHPDILSIATSRALLNAAAAGLLAPEVAEELVATLKLWRRVQGFLRLTTDGVLDPRQVSPTLREGLSRAAFPDEEPAVDFAALDSRIRDIAARAHRHFVALVEEPASRLPPPETNEEAKLP

SEQ ID NO: 23 - protein, 470 aa, synthetic construct
MRALRTDAAREHLARIVALLIEQLSHGGDPDGALNAMDSFLRDLPGPHLLAALEHNPDLVRLIATIVSAAPRLGETLARRPSLADALLDPAFFDVLPDEPALAAHLEALLDAAPNEEEQFDRARRFRQEQHVLIGVRIVSGTLPAARAGEAYARVAEVIIRALHARVWARFVEAHGSIPGADTAVLSMGKLGSREMTAGSDLDLIVLYDFDPASDGTSDGPRPLVGAQYFARFTQRLVTALTSLTNAGKLYDVDLRLRPSGRSGPVATRIGSFENYQRTEAWTWEHMALTRARIISASPAFARRVEQVILQVLAHPRDPRRIAGDILDMRRAIAAEKGESDRWNLKHAAGGQVDAEFLAQFLVLIHAERHPEIVDTATARILTVAGWLQLLSPEDCQTLSNACRLYQDLTQVLRLAIDRPFVPAQASPALKALLARAGEMPDFSSLDAHLTDTEARVRAIFERILEAASA

SEQ ID NO: 24 - protein, 460 aa, synthetic construct
MHLARIVALLIEQLSHGGDPDGALNAMDSFLRDLPGPHLLAALEHNPDLVRLIATIVSAAPRLGETLARRPSLADALLDPAFFDVLPDEPALAAHLEALLDAAPNEEEQFDRARRFRQEQHVLIGVRIVSGTLPAARAGEAYARVAEVIIRALHARVWARFVEAHGSIPGADTAVLSMGKLGSREMTAGSDLDLIVLYDFDPASDGTSDGPRPLVGAQYFARFTQRLVTALTSLTNAGKLYDVDLRLRPSGRSGPVATRIGSFENYQRTEAWTWEHMALTRARIISASPAFARRVEQVILQVLAHPRDPRRIAGDILDMRRAIAAEKGESDRWNLKHAAGGQVDAEFLAQFLVLIHAERHPEIVDTATARILTVAGWLQLLSPEDCQTLSNACRLYQDLTQVLRLAIDRPFVPAQASPALKALLARAGEMPDFSSLDAHLTDTEARVRAIFERILEAASA

SEQ ID NO: 25 - protein, 450 aa, synthetic construct
MEQLSHGGDPDGALNAMDSFLRDLPGPHLLAALEHNPDLVRLIATIVSAAPRLGETLARRPSLADALLDPAFFDVLPDEPALAAHLEALLDAAPNEEEQFDRARRFRQEQHVLIGVRIVSGTLPAARAGEAYARVAEVIIRALHARVWARFVEAHGSIPGADTAVLSMGKLGSREMTAGSDLDLIVLYDFDPASDGTSDGPRPLVGAQYFARFTQRLVTALTSLTNAGKLYDVDLRLRPSGRSGPVATRIGSFENYQRTEAWTWEHMALTRARIISASPAFARRVEQVILQVLAHPRDPRRIAGDILDMRRAIAAEKGESDRWNLKHAAGGQVDAEFLAQFLVLIHAERHPEIVDTATARILTVAGWLQLLSPEDCQTLSNACRLYQDLTQVLRLAIDRPFVPAQASPALKALLARAGEMPDFSSLDAHLTDTEARVRAIFERILEAASA

SEQ ID NO: 26 - protein, 237 aa, synthetic construct
MSTKKKPLTQEQLEDARRLKAIYEKKKNELGLSQESVADKMGMGQSGVGALFNGINALNAYNAALLAKILKVSVEEFSPSIAREIYEMYEAVSMQPSLRSEYEYPVFSHVQAGMFSPELRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 27 - protein, 218 aa, synthetic construct
MARLNRESVIDAALELLNETGIDGLTTRKLAQKLGIEQPTLYWHVKNKRALLDALAVEILARHHDYSLPAAGESWQSFLRNNAMSFRRALLRYRDGAKVHLGTRPDEKQYDTVETQLRFMTENGFSLRDGLYAISAVSHFTLGAVLEQQEHTAALTDRPAAPDENLPPLLREALQIMDSDDGEQAFLHGLESLIRGFEVQLTALLQIVGGDKLIIPFC

SEQ ID NO: 28 - protein, 207 aa, synthetic construct
MSRLDKSKIINGALELLNGVGIEGLTTRKLAQKLGVEQPTLYWHVKNKRALLDALPIEMLDRHHTHSCPLEPESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGS

SEQ ID NO: 29 - protein, 360 aa, synthetic construct
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 30 - protein, 200 aa, synthetic construct
MARTPSRSSIGSLRSPHTHKAILTSTIEILKECGYSGLSIESVARRAGASKPTIYRWWTNKAALIAEVYENESEQVRKFPDLGSFKADLDFLLRNLWKVWRETICGEAFRCVIAEAQLDPATLTQLKDQFMERRREMPKKLVENAISNGELPKDTNRELLLDMIFGFCWYRLLTEQLTVEQDIEEFTSLLINGVCPGTQR

SEQ ID NO: 31 - DNA, 70 bp, synthetic construct
  source 1..70: mol_type = other DNA
attatcaccgccagaggtaaaatagtcaacacgcacggtgttagatatttataaatagtggtgatagatt

SEQ ID NO: 32 - DNA, 55 bp, synthetic construct
  source 1..55: mol_type = other DNA
tccctatcagtgatagagattgacatccctatcagtgatagagatactgagcaca

SEQ ID NO: 33 - DNA, 67 bp, synthetic construct
  source 1..67: mol_type = other DNA
taatgtgagttagctcactcattaggcaccccaggctttacactttatgcttccggctcgtatgttg

SEQ ID NO: 34 - DNA, 56 bp, synthetic construct
  source 1..56: mol_type = other DNA
gattcgttaccaattgacatgatacgaaacgtaccgtatcgttaaggttactagag

SEQ ID NO: 35 - protein, 605 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAA

SEQ ID NO: 36 - protein, 246 aa, synthetic construct
MNNVIPLQNSPERVSLLPIAPGVDFATALSLRRMATSTGATPAYLLAPEVSALLFYMPDQRHHMLFATLWNTGMRIGEARMLTPESFDLDGVRPFVRILSEKVRARRGRPPKDEVRLVPLTDISYVRQMESWMITTRPRRREPLWAVTDETMRNWLKQAVRRAEADGVHFSIPVTPHTFRHSYIMHMLYHRQPRKVIQALAGHRDPRSMEVYTRVFALDMAATLAVPFTGDGRDAAEILRTLPPLK

SEQ ID NO: 37 - protein, 385 aa, synthetic construct
MLTVKQIEAAKPKEKPYRLLDGNGLYLYVPVSGKKVWQLRYKIDGKEKILTVGKYPLMTLQEARDKAWTARKDISVGIDPVKAKKASSNNNSFSAIYKEWYEHKKQVWSVGYATELAKMFDDDILPIIGGLEIQDIEPMQLLEVIRRFEDRGAMERANKARRRCGEVFRYAIVTGRAKYNPAPDLADAMKGYRKKNFPFLPADQIPAFNKALATFSGSIVSLIATKVLRYTALRTKELRSMLWKNVDFENRIITIDASVMKGRKIHVVPMSDQVVELLTTLSSITKPVSEFVFAGRNDKKKPICENAVLLVIKQIGYEGLESGHGFRHEFSTIMNEHEWPADAIEVQLAHANGGSVRGIYNHAQYLDKRREMMQWWADWLDEKVE

SEQ ID NO: 38 - protein, 92 aa, synthetic construct
MVVTTSDVVMCQMRRSDVQGGYRVYGSWMAENVQDQVSILNQKLSEFAPSMPHAVRSDVINNRLQNLHLHAHHFLIRRHQLITHLNPHLHRN

SEQ ID NO: 39 - protein, 368 aa, synthetic construct
MSVKKLENGQYQVDVSFGFDPITGERIRTRPVASTRKEALELEAKLRREFQEERARKSRSVSFPTLISIYLASCEIDSKPNYYQNQKYIINKHISDYFLKSDIQKITHREITDFRKHLMETGLSNKSVNNIMTSLSKIFDTAVHEEILKRNPCDNVKRLPLTRKKMKFWRPEEFKKFISLIPQDQLLFKTFYTVAFLTGLRCGEMLALQWKDIDKILLEIDVHKSCTWLDGQFVVTTPKTKNSIRRVSINKKLLKLLERWKEAQEELFNELGIRHSHDTYIFQYKDTPSRKDIFSRKIKYFCKDSDLTPIRLHDFRHSHVALLIHQGEDYITIKERLGHGSVKTTIDVYGHLYPNKQKEMADKLDDLL

SEQ ID NO: 40 - protein, 197 aa, synthetic construct
LDRLRAVLDQGRNPKLYLQQERAKYSANQSFESIFRDWIDSAGKQGLKEKTWHYQKRSSEIYLLPRLGKYPLTDITELSLRNCLREVSESSPSNTERLVSVLHKFYDWLIDEQILEINAAAGITAKKVGGKKGKRTRVLNDNEIRILWRYLHESKITEKNRIYIKLLLLLGGRKGELIQAEKHHFDLQSAMWTVPIE

SEQ ID NO: 41 - DNA, 1818 bp, synthetic construct
  source 1..1818: mol_type = other DNA
atggacacgtatgcgggcgcatacgatcgacagagccgtgagcgtgaaaatagctcagccgctagcccagcaacccaacgatctgccaacgaggataaagcagctgatctgcaacgggaggtggagcgtgatggcggacggttccgctttgtgggtcacttctcggaagctccgggtacatccgcctttggtactgctgaacgcccggaatttgaacgtattctgaatgaatgtcgcgcgggccgcttaaacatgattattgtatatgatgtcagccggtttagccgcttgaaggtgatggatgctatacctattgtcagtgagctgctagcattaggcgtaacaattgtctcgacccaggaaggcgtttttcgccaaggtaacgttatggatctgatccacttaattatgaggttagatgcatcgcataaggaaagttcgctcaaatccgctaagattttagataccaaaaacctccagcgtgagctgggtggctatgtgggcgggaaagcgccgtatggctttgaattggtcagcgaaactaaagagatcaccagaaatggtcgtatggtcaacgtggttattaataaacttgcacattcaaccacgccactgacaggtccgtttgagtttgaaccggatgttattcgttggtggtggcgcgaaatcaaaactcacaaacatctgccgtttaaaccgggatcacaggctgccatccatccgggctccatcacgggtctgtgcaaacgtatggacgcagacgcggtgcctacccgcggcgagacgatcggcaagaaaaccgcgagcagtgcgtgggacccggccactgttatgcggattctgcgggacccgagaattgccggcttcgcggccgaagtgatatacaaaaaaaaacccgatggcactcccacgaccaaaatagaaggttatcgcatccagcgagatccgatcacccttcgtccagtagagcttgattgtgggcctattattgaaccggctgagtggtacgaattacaggcgtggctggacggtcgtgggagaggcaaaggcctctctcgtggtcaggccattttgtctgcaatggataaactctattgcgaatgcggtgcagttatgacgagtaagcgtggtgaggaaagcatcaaagattcctatcgctgccggcgccgcaaagtggtggacccgtcggcccctgggcagcatgaaggaacatgtaacgtatccatggcggcgcttgacaagttcgttgcagagcgcatttttaataaaattcgtcacgcggaaggcgacgaagaaacactggcgctgttgtgggaagcggcccgccgcttcggtaaactgacagaagctccagaaaagagtggagagcgcgcgaatctggtggcagaacgtgcagatgccctgaacgcgctggaagaactttacgaagatcgtgccgccggtgcgtacgatgggccggttggtcgcaagcatttccgcaaacagcaggcagccctgactctgcgtcagcaaggcgcggaggaacgcctggcggaactggaagccgcggaggctccgaagctgccgttggatcaatggttcccagaagatgcggacgcggacccaacggggcctaaaagctggtggggacgtgcctctgtcgatgataaacgagtgttcgttgggctatttgtagacaaaatcgtggtcaccaaatctaccaccggccgtggccagggcacgcctatcgaaaaacgtgcatcaatcacctgggcgaaaccccccacggacgatgacgaggatgacgcccaagatggaaccgaagatgtggcggcctaa

SEQ ID NO: 42 - DNA, 50 bp, synthetic construct
  source 1..50: mol_type = other DNA
ctacgcccccaactgagagaactcaaaggttaccccagttggggcactac

SEQ ID NO: 43 - DNA, 53 bp, synthetic construct
  source 1..53: mol_type = other DNA
ccgcggtgcgggtgccagggcgtgcccttgggctccccgggcgcgtactccac

SEQ ID NO: 44 - DNA, 35 bp, synthetic construct
  source 1..35: mol_type = other DNA
gctagcacaatacctaggactgagctagctgtcaa

SEQ ID NO: 45 - protein, 242 aa, synthetic construct
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 46 - protein, 241 aa, synthetic construct
SKALLKGVRDFNPISACVCLLENSSDGHSERLFGIGFGPYIIANQHLFRRNNGELTIKTMHGEFAVANSTQLQMKPVEGRDIIVIKMAKDFPPFPQKLKFRQPTIKDRVCMVSTNFQQKSVSSLVSESSHIVHKEDTSFWQHWITTKDGQAGSPLVSIIDGNILGIHSLTHTTNGSNYFVEFPEKFVATYLDAADGWCKNWKFNADKISWGSFTLVEDAPEDDFMAKKTVAAIMDDLVRTQ

SEQ ID NO: 47 - protein, 242 aa, synthetic construct
MGVSLSRGVRDYNAISSMVCRVTNDSGSSSTTMYGIGYGCYIITNKHLFRENNGRLLITSHHGEYICKNSASLKLSLVPGRDMLLIRLPKDCPPFPSKLKFREPTSEEKAVLVVTNFQEKHLSSMVSESSCVVQREDSPIWRHWISTKDGHCGAPIVSIRDGYIIGSHCGENPMTSNFFTSIPKDFQNLLNGKEANEWVSGWKYNIDAVCWGGLSVVNDAPSEPFITAKVVSALDTEGIKVQ

SEQ ID NO: 48 - protein, 219 aa, synthetic construct
SNSMFRGLRDYNPISNNICHLTNVSDGASNSLYGVGFGPLILTNRHLFERNNGELIIKSRHGEFVIKNTTQLHLLPIPDRDLLLIRLPKDVPPFPQKLGFRQPEKGERICMVGSNFQTKSITSIVSETSTIMPVENSQFWKHWISTKDGQCGSPMVSTKDGKILGLHSLANFQNSINYFAAFPDDFTEKYLHTIEAHEWVKHWKYNTSAISWGSLNIQA

SEQ ID NO: 49 - protein, 219 aa, synthetic construct
SKSVYKGLRDYSGISTLICQLTNSSDGHKETMFGVGYGSFIITNGHLFRRNNGMLTVKTWHGEFVIHNTTQLKIHFIQGRDVILIRMPKDFPPFGKRNLFRQPKREERVCMVGTNFQEKSLRATVSESSMILPEGKGSFWIHWITTQDGFCGLPLVSVNDGHIVGIHGLTSNDSEKNFFVPLTDGFEKEYLENADNLSWDKHWFWEPSKIAWGSLNLVE

SEQ ID NO: 50 - protein, 219 aa, synthetic construct
SKSLFRGLRDYNPIASSICQLNNSSGARQSVMFGLGFGGLIVTNQHLFKRNDGELTIRSHHGEFVVKDTKTLKLLPCKGRDIVIIRLPKDFPPFPKRLQFRTPTTEDRVCLIGSNFQTKSISSTMSETSATYPVDNSHFWKHWISTKDGHCGLPIVSTRDGSILGLHSLANSTNTQNFYAAFPDNFETTYLSNQDNDNWIKQWRYNPDEVCWGSLQLKR

SEQ ID NO: 51 - protein, 182 aa, synthetic construct
APITAYAQQTRGLLGCIITSLTGRDKNQVEGEVQIVSTATQTFLATCINGVCWTVYHGAGTRTIASPKGPVIQTYTNVDQDLVGWPAPQGSRSLTPCTCGSSDLYLVTRHADVIPVRRRGDSRGSLLSPRPISYLKGSSGGPLLCPTGHAVGLFRAAVCTRGVAKAVDFIPVENLETTMRSP

SEQ ID NO: 52 - protein, 233 aa, Homo sapiens [sequence omitted]

SEQ ID NO: 53 - protein, 315 aa, Homo sapiens
QWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMV

SEQ ID NO: 54 - DNA, 714 bp, synthetic construct
  source 1..714: mol_type = other DNA
atgggtgaatccctgttcaaaggcccgcgcgattataacccgatctcctcaacgatctgtcatctcacgaatgagtccgacggccacactacgtctctttacggtatcggattcggcccgttcatcattacgaacaagcacttgttccgccgtaacaacggtaccctgcttgtacaatccctgcacggtgtgttcaaggtgaagaacacgaccaccctgcaacaacatctgatcgacggccgcgacatgatcatcatcaggatgcctaaagatttccctccattcccgcagaaactcaaatttcgcgaaccgcagcgcgaagaacgcatctgcctggtcaccactaatttccaaaccaagtccatgtcgtccatggtctctgacacctcatgtacattcccgtcgtcagatggaatcttctggaagcattggattcagactaaggacggccagtgcggttccccgcttgtcagcaccagggatggtttcattgtgggtatccacagcgcctcgaattttacgaataccaacaattacttcacctcggtcccgaagaactttatggaacttctaacaaaccaagaggcccaacaatgggtttcgggatggcgcctgaacgcggattcggtgttatggggcggccacaaagtgttcatggtcaagcccgaggaacccttccagccggtgaaggaggccacacagctgatgaactaa

SEQ ID NO: 55 - DNA, 657 bp, synthetic construct
  source 1..657: mol_type = other DNA
atgagtaaggcattgctgaagggagtccgcgacttcaacccgatctcggcttgtgtctgcctgttggagaactcctcggatggccacagcgaacgcctgttcgggatcggcttcggcccgtatatcatcgccaatcagcaccttttccgccgaaacaacggcgagctgacgatcaagacaatgcacggtgaattcaaagttaagaactcgacccagctgcaaatgaagccggttgaaggacgcgacatcatcgttatcaagatggctaaagacttcccgccgttcccccagaagctgaagttccggcaaccgacgatcaaggaccgcgtttgcatggtgtccaccaatttccagcaaaagagcgtgagtagcctcgtttccgaatcgagccatatcgtgcacaaagaggatacttcgttctggcaacattggatcactactaaagacggtcaatgcggaagtccgcttgtgagcatcattgacgggaacatcctgggaatccacagcttgacacacaccactaacggttccaactattttgtggaattcccggaaaagtttgtggcaacctacctggatgccgccgatggctggtgtaaaaactggaaattcaacgcggataagatcagttggggatctttcacattggtcgagtaa

SEQ ID NO: 56 - DNA, 729 bp, synthetic construct
  source 1..729: mol_type = other DNA
atgggcgtttcgctgagtcggggcgtccgcgactacaacgccatctcttcgatggtgtgccgggtgacaaatgattccggctcgtcgtctaccacaatgtatggtattggttacggctgctacatcatcaccaataagcatctgttcagggagaacaacggccgactgctgattacatcccatcacggggagtatatttgcaagaattcggcgagtctgaagctgagccttgttccaggtcgtgatatgctgctgatccggctcccaaaggactgccccccgtttccgagcaaactgaagttccgggagccaacctcggaagagaaggcggtgctggtcgtcaccaacttccaggaaaaacacctgtcgtccatggtgagcgaaagcagttgcgtcgtacaacgggaagactccccgatttggcgccactggatctccaccaaggacggccattgcggcgctccgattgtgtcgatccgggatgggtatatcatcggtagccattgcggcgaaaaccctatgacgagcaactttttcacgtcgatcccaaaggattttcaaaacctgctgaacgggaaggaggcgaacgagtgggtgtccggctggaagtataacatcgatgcggtctgctggggtggccttagcgtggtcaatgacgccccgtccgaaccttttattaccgctaaggttgtttccgccctggatacggaaggaatcaaggtccagtaa

SEQ ID NO: 57 - protein, 382 aa, Bacillus subtilis
MKVPKTMLLSTAAGLLLSLTATSVSAHYVNEEHLFKVTAHTETDPVASGDDAADDPAIWVHEKHPEKSKLITTNKKSGLVVYDLDGKQLHSYEFGKLNNVDLRYDFPLNGEKIDIAAASNRSEGKNTIEVYAIDGDKGKLKSITDPKHPISTNISEVYGFSLYHSQKTGAFYALVTGKQGEFEQYEIVDGGKGYVTGKKVREFKLNSQTEGLVADDEYGNLYIAEEDEAIWKFNAEPGGGSKGQVVDRATGDHLTADIEGLTIYYAPNGKGYLMASSQGNNSYAMYERQGENRYVANFEITDGEKIDGTSDTDGIDVLGFGLGPKYPYGIFVAQDGENIDNGQAVNQNFKIVSWEQIAQHLGEMPDLHKQVNPRKLKDRSDG

SEQ ID NO: 58 - protein, 103 aa, unidentified
MKFFNYILFLIIINISYSQILFVFQHIRHGAREPIYYDRTRKDKNVDYYGYQWTGKAVLTPIGKRMNYLLGIRNRNKYKNLLNEIYDSREIFVSSTGFDRTIN

SEQ ID NO: 59 - protein, 114 aa, unidentified
MKLGTQSEGCVVDDRTGILYVAEEDVGLWRIDPGSTVPVRIAAADGRQIVADAEGVALAPVGERGGYVVVSSQGDNAYTLYSLPDERYAGRFRVVDGPRSADRRRPGRHRTDAR

SEQ ID NO: 60 - protein, 162 aa, Clostridium colicanis
MGLNYSQVIARNNAQLSSIKIGEPLTIKNTTIVPKEVYSEETLIKENNNQYIRIPVTDTKLPTPEMVDLFVSSVTNLPKNSWLHFHCKEGIGRTTTFMTMFDMMKNAKTVPLNDIVLRQIKLANLENKEKGLESTTRMAFYNKFYAYAKNGDFTKKFSEFNN

SEQ ID NO: 61 - protein, 164 aa, Chlorobaculum limnaeum
MATKVRAFGAWRGVKEIEAVAVDDGNATVYYSDERFGIRKYSADPAAVDAGKELAVFGQSGFAADREGIAVTRNAEGGTLVLVSDQSGGALRIYSSTKVDENRYRFITSVPYRATETDGLDLSTEIRTPEFPEGVLVAMSDDRTFQFYSLGEVLKRLGERVDGE

SEQ ID NO: 62 - protein, 175 aa, Bacillus amyloliquefaciens
MNHSKTLLLTAAAGLMLTCGAVSSQAKHKLSDPYHFTVNAAAETEPVDTAGDAADDPAIWLDPKNPQNSKLITTNKKSGLVVYSLEGKMLHSYHTGKLNNVDIRYDFPLNGKKSILRRHPIGLKERIPLRFTPLTGKTAHYKALRIQTARLHQQLMKYTVSACTTVKKQENITRW

SEQ ID NO: 63 - protein, 235 aa, Cystobacter fuscus
MGGAALRALAANTPGPLVVVDLREESHGFLGDLPVSWYAPRNVGNRGRTREATLAEELRLLDSLRRHESLAFDGQGKDRGPPEPVRPIAAFGTVCTEESICTEAGAGYARLLVTDHHGPDAGELDRFVAFLERLPDGAWVHYHCRGGRGRTSTFLLLHDLLRNAHRLPFSVIAHRQRVLSDGYDLLAHGEPADWKTPLRRARAEIVPAFAEFARERAVGGSQRFTEWLGARQEMR

SEQ ID NO: 64 - protein, 260 aa, Acinetobacter baumannii
MNILFKMTVLAASLLLVACNDNDDQEAQTAPSTNQSKYYQTKTPYQPQQEIKNYEQTPNGFQPVFTELVARHGSRGLSSLKYDLALYNLWKQAKAENALTPLGEQLGADLEAMMKANILLGYGVEGIRQYGYGNETMTGILEHRGIADRLLQRLPTLLNSQASILVQSSGVDRAVDSAKFFTAELIKQRPPLKDKIVPLSYTNLSSESVPSVEDGGVDRFKLYFHSLNADEDLVQPLSASQQKFMMLARPIKILKKIIKI

SEQ ID NO: 65 - protein, 268 aa, unidentified
MRWVKAVLTFGLLGCLLKGKPLVPKPPCFVKDPTSAKRFQTRLRVLSPRNPTLTKNLQRLRVIGSPQPSFKQLKMLLQENPQLYGIVDLRKETHFFMKGQSISWGTKGNCVNHRTIQEHLSEHAWTELQGLKYLRLHVPDHYPPTIEQINTFLQFYKSLPPGAVLLIHCRGGRGRTTTFLTLVDIIENAKCDSFDTILARQWRLGGADLRKPRTGVKKRPAETRLKLLKLFYARVRKDAEGSLRPFQRQRRKTQLPEALSLPPPSVLI

SEQ ID NO: 66 - protein, 280 aa, Clostridium ihumii
MSYFKKLLLVLLLIFSFAFNSFSVLGYDEASNIYTVLDNENSNSLPNLFRKDASLNISGSAQFTAAQVENIINNIDSNKLYIVDLRQESHGLLNENIAFMFWNPNVDLNDGLSSSEVLRVESKEISKIPISSVMHIYNKKLDLLKTVKVQSVLSEKNLVTSKNINYIRFAVKDNYIPSDDIVDEFVDFVKSEGTSSHLHFHCEAGEGRTTTFMAMYQMMNNKDNLSLDEILQYQIKLGGISLIDIPIRKDFLNTFYNYTKANIDSNFEVPYSKWVERKVI

SEQ ID NO: 67 - protein, 283 aa, Desulfovibrio fructosovorans
MESDPTLIFDSIREQGLPSHFRSLAGPWAVPITTTPPSRQGLAELRASGSEQPSSSEMATTIKGLAPEVTVVDLRQESHALLGEHPVSWYGLRNWANDGKSLQAVECDEETRIGDLPPCGEAAVSRVISKDPDGALAEVRVEEVAYDRARSEQEALCGLGLGTFRIAVRDHSRPSDADVDRFIRFVRELPPGTWLHFHCHAGDGRTTTFLLLYDMLRNAAVLGLEELAARQHMIGGIDLLHTPHTGWKGALYNERAAFVGRFHHYAGTRDFRRVLWTQYLASA

SEQ ID NO: 68 - protein, 285 aa, Clostridium novyi
MKKTNFSFTLFILLFLISIPVNGKLISTDAPVPPKPLVVLDSNSTSPLPSFFRIIPELNISGSKQFTPAQLKNIQNKINSKNLYIVDLREESHGFINDNTAISFYLPRKYINDNFNTHEILKKEKLDLDSIKNVENLNIYDQLGTLKETLKPDKTLSEKELTKHNKIHYVRLPVIDNYIPSPEIVDKFIELIKSKPSNSHLHFHCKEGQGRTTMFMAMYEMMHNKDNLSLDEILKHQQDIGGIILTGNPARGTFLKNFYNYTIDNKKNNFDTSYSQWLKNISSMK

SEQ ID NO: 69 - protein, 291 aa, Clostridium bornimense
MAKKIHFNFIKIFIVLIIFSLALPNLLVNGKSPSSDIHIVLDTLNNTEIPRNFRSTNNINVSNSINTKGMENLNISGSSQFSKTNLPLLIKSINTNLPIIDIDLRQESHGFVNEMPISFKNDKNNANLGLSSEEVLLKEIKDLNTINLNSTLTFYNDKNNPVLVKSVENESLVISKNSIGYLRIAVTDSMLPNKDEVNKFVTFVKNQPKNTHLHFHCKEGIGRTTIFMILYDMMKNYNDIPMEDIISRQIALAKLDEKDYMAFYDKNHTEFFTSFYNYCRSSKDLFLSPWQ

SEQ ID NO: 70 - protein, 297 aa, Bacteroides stercorirosoris
MFCPVSILWGQDRIQQYAGTAMPYPIVKDSFVFFQDSMVPFYVNHLGRHGARFPTSGKALNRVKEILELARRENRLTSGGVTLLSTIQNLSDTFDGQWGKLSVVGEEEQRGIARRMIERYPQLFSDSVKVQAIATYVPRCIHSMDAFLACMVEFNSSLHIQRNEGKQYNDILRFFDLNQSYVDYKENGDWRPIYETFVRRKISPASVMENFFLESGQETDKEAEEFVMALFSIAAILPDTGTPINLDGLFTIGEWDNYWQTQNLRQYMSKSSSPVGRMLPVAIAWLCYQNLFIQLMK

SEQ ID NO: 71 - protein, 303 aa, Clostridium paraputrificum
MKKYLKIISSILIFSLLSPLSISAKEVEQVYLSLDYENTDSLPHNFRKTTDLSNVIKGGGNTTGLENLNISGSSQFTSLSFLNLKKNINTKNEFWDIDLRQESHGFINGNAVSWYGPGDKANAGLNYKEVIAKEAKQLSEIPFGKSITLDNGKHTLIPTVVENESKLVSSNNVKYLRIAVTDGDRPTDESVDQFVKFVTHLPNNYWLHFHCKEGIGRTTTFMSLFDMMKNSKNVSLDDIIKRQFLLGGFNLFKPGDENKRVLFLRNFYKYTKENNDNFKTTWSDWIKQNNITPYTSTTELPKN

SEQ ID NO: 72 - protein, 304 aa, Clostridium pasteurianum
MRKRIKLFWITFFIAIFSLSFIFENVNAVSFAPKELRLTLNANNTVTLPKNFRKTTDSDKIKDIDKSVNLEGMNKLNISGSGQFSEKGLEMAKENIGEKVPITVVDLREESHGFLNGNAISWTDGHNKANKGLIEAQVIKDENERLKKLSEEKTVEIKNRTLNVEKVENEENLTKKHGISYTRITVTDKEAPSKEAVDEFVNFAKSVPNSGWLHFHCKAGKGRTTTFMAMYDMMKNAKNVSFEDIIKRQFLLGGENLLKRTTVENIKGTRAKFLKNFYDYCRTNNDNFNTTWEQWLKNNPDNSR

SEQ ID NO: 73 - protein, 308 aa, Clostridium perfringens
MFKYFKKNALIIFLVLSIFTSFFINNTSIVLAEDNNVHLVLDTKNINNLPNNFRTTSDLERLKNLSNINTKGLDTLNISGSQQFSPNNLSLLVTSIKTNLPITVVDLRQESHGFINEYPVSWKGTKNAANLGLTREEVIDTERNLLNSITLGTPIQFFNNPKLTVIPEKVLSENQLIKANSMDYIRIPVTDGKLPTYEMVDFFVQYINSMPKDSWLHFHCKEGIGRTTTFMIMYDIMKNYNNATLDEIIYRQLVLFGLTEKNFNSFLSKERLDFFTKFYQYVQEENTDFKTSWSQWLNKNNFPLVTIR

SEQ ID NO: 74 - protein, 308 aa, Clostridium botulinum
MYKKAKLKLYIPMLLLITIFSSFFLNFNIALAFNEPNNKSDVHIIVDNLRTNKIPSNFRTTSNLTNIKNNSSLNLKGLETLNTSGSQQFSKDNLDILTKSIDSTLPILVIDLRQESHGFVNEFPISFANEKNDANLGLSKSAVTFTEKKDLKSIKLNTPLTFYKHPEINVVPKEVLSEKQLTKSYSLNYSRVPVTDTKLPTNEMVDCFINIVKECSKENWLHFHCKAGFGRTTTFMIMYDMIKNYNNATYDEIIKRQFALANFDEKEIIELSSSDRINFLNQFYNYCKDVNGNFDTTWSSWLNDSQKH

SEQ ID NO: 75 - protein, 311 aa, unidentified
MKNIYILLFLFSLGVVAQSFLYQHQPTLFPIVDAPLNSENHLPKKWRSTKQISQLVGNYANLKGLTQLHLSGSGQFSQEDLKNMSQEIKGKAFVLDLREESHGFIDGTPISWTDGLNYGNVGKTLRQIELDEQKRLKLTAQKGSIIVDLSKDLGENFQKFFVREVKTEKELVESFGYTYIRLPITDHHRPVDSVVDQFIEIVLSLPADSWIHLHCKGGKGRTTTFMTLYDIMHNAQTVGLNDILSRQTLLGGADLVQAEKNETYKQKPAKDRIEFIRAFYTYCREVPNFEMTWSDWVHQKQLVAYQTSSSN

SEQ ID NO: 76 - protein, 314 aa, Acidovorax avenae
MPHVSLACRHHAGTRPDRSAGRTALQLRQPEAETRLELVYDTEPPHPAGTMAFFRHSTQASELPPGIDTRGLESLQLSGSERITSVEQVRAIRQACGDAPLVVVDLRQESHAVADGHSLTWRGPMDWGNVGLGTAAATAREAEQLEELRRQGNAVATHADHVKGKSDEPALRRLDTTLARSEQEIVEAAGADYRRIAVTDHLRPSRGEVDQFIDLVRGLPDGAGLHVHCNGGRGRTTTFMVLYDMPRNAREAGADAIMARQSRLGMDYNRCAYRPPDACGFQVKFSERTLAVVRTSWRSPINPLIPRVSPIDCS

SEQ ID NO: 77 - protein, 314 aa, Clostridium sp.
MKKSTAVFFAFILVFISLSSTTLSLDTAAVAASDEVQLALDSLNESTPRNFRKTSTPIYLTKNEKFSLKGLDKLNISGSQQFSEKNLPILLEEIGTSKPITIVDLRQESHGFINGLPVSWKNLNNDVNKGLTRDEVIEKEANQLSSIPLNSPVTVYRAPNNIKIPTEVFNEDALVTSKNLSYIRVPVTDLGLPTDDMVDYFIEVFKNLPKDSWVHFHCKAGVGRTTTFMIMYDMLINYNQVSATDIIDRQLKLANFDEVTIREFYTPERYKFLLNFYNYCKENGGSFKMSWSTWSKEYDGFISLNKDTGYLKVS

SEQ ID NO: 78 - protein, 325 aa, Clostridium sp.
MKQNEITLNNSTNYNTIEDNFRHGHHGWYSYRHPFLYEYCYHINDEMIQEPYYKSRSNKLHLVINSDNAKKLPDNFRNTIDLANFKDNKVINLNGLANLNASASSQFSESGLAFVKQSIGHAMPIIIVDLRQESHGFINGVAISWADNHNKANKGLTKEAVLTDENTRLHGILLNKPIYIGDITLIPKKLENEKSLVQSYDMSYMRIPVTDNEMPNNGMVDYFIKFVNSLLPNTWLHFHCKAGLGRTTTFIVMYDMMKNAKNVSLQDIMNRQVLLGGKNLLRDEQLLMNRSEQRVKLIKRFYRYCVENNDNFKTTWSQWIRQHRN

SEQ ID NO: 79 - protein, 332 aa, Acidaminococcus sp.
MLRVGGLFFIGGKMLKKVPFLLMLSASLIAFSAPLPCVRAKEAPKSVPLEAPFSYEGALWRLDAENAPGLPRNYRTCDDAYRAIPPRYAKEASSRIPSCKGLSELHISGSSQYSEKGLDAILADLRNRTQGPIVLVDLRQESHGFVNGMPVSWYGKRNWANREKGHFAVLADEAQRIHALQGQDVTLVRLKTDKRSASTMTVEAGQVLTEAELAAQKGVMYVRFTATDHLWPDAGEIERFRRFVKTLPKDAWLHFHCVAGEGRTTAFMTMYDMLKNPDVPYEDIVLRQLRIGGVYTPFLGKGLSTWKKPYYRAKKRGLEAFYRKLHKTQVTD

SEQ ID NO: 80 - protein, 332 aa, Anaerovibrio lipolyticus
MLMKKNRIEKSACSIFAVVCSLLIMLMATAQAAEKQEAPIWRLDSKPGAVLPRSLRFMTDEFPQDLNSVPSRKGMDTLRCSASAEFSGFGLSMIRDKIYEHAGKNAVIYMVDLRKESHGFVNGEFAVSHYFKKNLGNRNLASAAVPQAEEKALQSIMGQEITFVPLGKTDTKLFTASTVKVEQIETEEAITARLGIHYKRIPIADQCAPSDEDIDEFMNFYKNLPSNSWLHFHCHAGHGRTTTFMVFYDILCNPDVALDDIVARQHALGGTNLFAPGKKNNWKGEEIRKRAQQVRNFYAYVQANHRNKYAQPYSQWMKAQNSRVDNAQKSKS

SEQ ID NO: 81 - protein, 339 aa, Cellulophaga sp.
MNKYINYIGVMVVVLAFACKENSLPAIAPNVITEKTKNDTDDPAIWVNLKNAEKSIVFGTDKETDGGVYAFDLNGKIIKNKSITGVKRPNNVDVAYNFKINDSTFTDVLVFTEREKQQIRMFSVPDMMPLDNGGFSVFKNEKNLEYTLPMGVGLYTSSVDNTLYAIVGRKNGPKDGYLHQYKLSTDTTGVVQSTLVRKFGVFSGKKEIEAIAVDSELGYVYYSDEQHGIRKYYAEPTKGDKEISCFGGELFLSDIEGIAIAKQAKGKGYIIVSDQQKGQFNIFSRDTNKFVKAVNLSTTETDGCEVVTVPLNNVFKNGLFVAMNDAKDFYFYDLAKLGL

SEQ ID NO: 82 - protein, 339 aa, Bifidobacterium dentium
MLMSTRMMKRIVGSVACVAAAAMLMPGTVCPALALESDGQYYSSKQPYVAPSAATVASYSKAPEGYEPIYTESMARHGSRGLSSYKYDALLMKMAEAAERDGGFKSDAIKTEFMKNLNGITAANVENGYGMLTGQGADQHYGIGERAYQRNQSLFDQAAVDGGTISYQSSGEARATESGENFEKGFNAASGGKLTDSVVKPTDPADSGDGKDFQKNPDTLYFHKVQNPDGTEKTPGTKAYDIANDYQNFISWPTMRSSPVPRRPSATMPTSSGPRMTCCRRSSPTNSLPNSRTANTRGTTPPMAPRRVPRTARRAPTRPRTPMRAAKAPRRSRASTTPQ

SEQ ID NO: 83 - protein, 344 aa, Chryseobacterium sp.
MKNIHYILALSVLPFVINCTGQKDLAEKLKPAVITETVVHDTDDPAIWINPQDATKSIIIGTDKDTDGGLYAFDLNGKIINKVSGLKRPNNVDLEYGFMLNGKKTDIAAVTERETNKVKLYTLPELKEVGEISVFDGESERGPMGISLYKNPQTEEIFAVVGRKSGPADGYLWQYKLIEKDGIITGEVVRKFGKYSGLKEIESIAVDDEMGYIYYSDEQFGVHKYYADPAKGNEELSVFGKGDFKSDVEGISIYPTSQGKGYILVSNQQNDTFNVYLREDQSKGKIAEIPVSTLESDGSEVTNVNLGPKFPKGVFVAMSNGRVFHFYDWRMVEKAITSAVKAVK

SEQ ID NO: 84 - protein, 347 aa, Erythrobacter litoralis
MLKPFHGFALISLGIAAACASIHVTGDPAVTVTAVAETEPVGTANEDAADDPAIWRNPADPAKSLIVGTDKKAGLHVYDLAGRELSFMPAPGLNNVDLVELPDGRVLALSSDRSDLETAHVSVARLDPDTAALTPIARIEVGPGEGYGICMGEVDGDGSFTVFSAPKKGIVYRTALQFEGEELIDKTETLTSVPSQPEGCIADPRTGTLYIGEEAAGIWAIDMQTGDKRLVAEVDNRLLVADVEGLAIAPEGRDGGYLVASSQGDNAYAVFRLPGMEPVGRFRIGAGAFGSTEETDGIELDPRAFGPAFPGGLFVAQDGMNGASAQNFKLVRWDAVLEALEAVPPAP

SEQ ID NO: 85 - protein, 350 aa, unidentified
MRLTFIVCASICLLGCSTTQRGPAWAGGETPVAVAALAETRVDTDPAVDADDPALWSDESDPTRAVMFGTDKTDGLYVHDLDGSVRQFLASGALNNVDLRTGFSAEGRDDFVLVAATNDERMGINLYLFDPRTLETRDYGFIETDIGEPYGFCMGRRSDGFYLIANNKQGDIKIWRLANGAEPGAAEMVRQLKLPSQLEGCVVDDDRDVLYVGEENAAIWRFDFDPAASPTPTKVASVDRQRITDDIEGLTIMRDGRQRYLIASSQGDDTFPVFRITEEGETYLGRFTVVARDDIDGVTHTDGLDAWSGPIGRYPEGALAIHDDNEEPLEGQQNYKIVDWREVKAALDLP

SEQ ID NO: 86 - protein, 355 aa, Devosia riboflavina
MIDLRPALALLALSLCWTGTALAQPEPIFASVSATLETENVDIDPDDPAIWINTADPAQSRVIGTDKDVGLVVFDLDGKIVQRLSDGRLNNVDIRQDVTVGNETLSLVAASRRNDDTIVFYTVDETGTLTKATPFAFPGAPRKIDDDIYGIGLYHDAVAHRLFVIASFKTGEIMQWEVTSNAGQLSLAFTRELAVPTQPEGVVADDELGFIYVGEEDGGLWRFAAAPDADASGHLVDKVGSACLPEDDLEGLAVVKGADNSSYILASAQGVNRYVLYARQPDADGNQPCYGAFNLVTGATDPVSETDGLDVTTTALSDRFPKGLMVVQDDRNEGFNRNFKFVSWADVMQAMQLAD

SEQ ID NO: 87 - protein, 360 aa, Bradyrhizobium sp.
MRCGPGKPVGLSYQMPAPDSDLGARAPASSSVESRTPFRIPPTTIPKKISDQNPPTLVYDMIEPSTFRTTSDTSGMESCDLTGFDGLSLSGSEQFSERQLEELITLTRPKRIVIIDTRQEFHGFANGEPISWYAADNYNWGNVEKPAEIIPAKETKKLAKLPTKFPFGLTIESGSVVKGKASSAPPKVLDFGKVKIETEEELVKRKGVAYLRIPTPDHARPTDAEIDKLVSELRNLARAGDVHLHFHCRGGIGRTTLLMTLADMLHNSDKVQCAHIIERQIKLRRRDEFPTSKALNKVLRNEKSELMNTFFQYAKEQPLEKADAISWTEWLALKAHREHQILDTSSVGTPPTAPPGHRMP

SEQ ID NO: 88 - protein, 362 aa, Algoriphagus marincola
MHKKLNYFLSIFTLTGLASCQESSLQKGDLPDEKVELISPLHISDSVIHDTDDPAIWINKSNPSQSLIIGTDKDSIGALYVFDLTGKTIDSLVRRDIQRPNNVDIGYGLIHGKDTIDFAVTGERLTSKIRFFSLPDMKELNPGGLEVYQGETGEDYRDLMGIALYQSPKSGKTYVIAGRKNGPTDGTYLWQYEVKSQDGNLSLELVRKFGNFSGNKEIEAIAVDNELGYIYYSDEGTGVRKYYAEPDMGNEELALFATEGFTDDHEGISIYKTSETEGYLLVSDQGANLFHVFPREGSQSNPHQHSLITTLPLSTLSSDGNEVTSADLGGEFSEGLFVAMSDDKTFQIYSWKQLKKHINQNQ

SEQ ID NO: 89 - protein, 369 aa, Bryobacter aggregatus
MKKNQTFLSFFLLAAYGIAQKESPQPKLQYAVILTRHGVRAPLAPNRWLHFYSVGSWPSWGVLPGHLTPHGRQMMKLMGAYYGETFRAEGLLARNGCEDSGRIYVWSDSDQRTTESARALLEGIIPGCRYAVHSGRPGIPDPLLSTQGTARFRSEDRSGLQPALDKLHFVLKGARARSNLSLRTAATLAENLMLEYANGFEGNELGWGRLSQEDLQEILTLRAAQPRKAEYARNLWLRIQTSLCQAATGESIDGSIGPKNTALLILSGHDKNLESLAALLGLNWQLASYPKNYASPGGALVFTLWRQDGRFRLRSQYIAQSLEQLHNGSGGTPVVIDLGSSDFTPITRPPHGHFCVATSLLVTSSKRRR

SEQ ID NO: 90 - protein, 371 aa, Asticcacaulis sp.
MDLKRFYVGFTTASAVVITATLLTGCASLHSELVSDSKLIAIGTGTPVLASAETVAVGTAGLDAADDPEIWVDPMDKSRSLILGTDKKAGLYAYNLDGSVRDFVAHGPLNNVDLRTIYGETGSFTLIAASDRARNGAALFTLTDDLKLKVAGFLPMTTSEAYGLCMGTIEAGITIVIIGKNGDVVQAIYSETEAGPRGEIVRRFNVGTQSEGCVIDDRTGALYIAEEAKGIWRYGVEPATGSTRVELQAAPSNILVPDVEGLALLVDEDSGLSYLIASSQGDSAFAVWQVAGETSIYKGRFSVFPGNGFDAVTGTDGVAALGGQVGPYAEGVVVMQDDSDMEGETPTGARARQNFKIVPWMDVKKALNIGS

SEQ ID NO: 91 - protein, 375 aa, unidentified
MGSNYTIYKRFFNSRNVKRLLIVLLFIGIVHVSFTWYLFHSESTNTITPTMKLLAEGSIDQDDMCIWIHPTESSKSVIITADKKAEKVFVYDLEGSVLQVLSMPKPGNIDVRYGFPLGGKKIDIVAYLQRKKGHRIIVFAVEPKTHQLKRVDDNSILVGKGVGGTLYHSQKTDKFYFIKTAEKKGKGGIEQFELFDDGKSKITGRKVRQWDLAGCEGAVADDESCALYISQEDKGVWKVGAEPGEPTPGELIIKTWKNGLMFDVEGLTIYKSHTREGYLMVSNQSRNSVKVYQLGKEDKFLGTFHIDGAIHTDGIDMTNVCLGIRFPKGLFVCHTDQEGSNRCPVLVTPWESIARGLNFKVDVSWNPRSETFKHF

SEQ ID NO: 92 - protein, 375 aa, Bacillus licheniformis
MNFYKTLALSTLAASLLSPSWSILPRAEASAYKDFSVTADAETEPVDTPDDAADDPAIWVHPKQPEKSRLITTNKKSGLIVYDLNGKQLAAYPFGKLNNVDLRYNFPLDGKKIDIAGASNRSDGKNTVEIYAFDGEKNKLKNIVNPQKPIQTDIEEVYGFSLYHSQKTGKFYAMVTGKNGEFEQYELFDNGKGQVEGKKVRSFKMSSQTEGLAADDEYGKMYIAEEDAAIWSFSAEPNGGDKGKIVDRAGGPHLTADIEGLTIYYGEDGEGYLIASSQGDNRYAIYDRRGKNDYVADFSIDDGKEIDGTSDTDGIDVIGFGLGKKYPYGIFVAQDAKIRKMDSQPIRTSKLSPGKKLLTRWTTSLISMIRSIPEN

SEQ ID NO: 93 - protein, 381 aa, Deinococcus proteolyticus
MSRFIRPLLLVSLCAGVCACAPVATGPASPQAQTGAGALPVVEARAQTAPVGDPADSDDPAIWVDAAEPARSFVIATRKEGGLTVFDLKGQTIQDLNPGGVRYNNVDLVRGFRLGGETVDLAVTSDRKGDRVAAFVIDPQTRTLREVTSPATPLLFSPGPGTDGKRTAYGLAAYRTAAGEDRVLVSQNGFPVVGEFELYDDGQGVSVRPVRRLELPAALPGLTVDDPQFEGMVVDAEQGVAFLGQEQIGVWRWDLDGRRSVLLDQVAPLAPRLHADVEGLTLVRGSGGRGYLLVSSQGSNAYAVYSRDGKRYFGSFQVTAGSDLVQDSDGADAVLTPLGSDYPGGLLVVQDGEAGGAEGQTNFKLVSWADVERALNLPDVR

SEQ ID NO: 94 - protein, 387 aa, unidentified
MNNNQYQLDKVVILSRHGIRTPLENTIAFLEKSSPFKWPSWDHAYGYLTTRGGVLETFFGHYLSQWLEQKNINIEPENPDIYVYANSLQRTVATAQFLVAGAFAGYDIPIHHKYTIEKMDPIFDPSVKNDSLEIKQKVLKEIEEADQKQSIFKNLAPAYKIVSDILDYPHSQLYAELKCDFADIPYELHFVKNEEPELRGPLAIGICVVDAFLLQYYSAFPKEQIAWGRITSQEQWQQLMHIRNQYIDLVFHSRTIARHNSKLLINKIDDLLHNKTHKVNLLVGHDSTIAALLGALDFAEYQLPNQFENTPIGGMVILQRYRNKASEKYLFKAEYVYQSFEQLYTAQPLDINHPPQHYQLQLKNAVANSDGFYQWQDIETRLKEFKK

SEQ ID NO: 95 - protein, 388 aa, Bacillus sp.
MLKEISTAALMTSVLFSTVNSAADLVGEFPSLSVQEKQRHSKFVKVEAKEETAAVANAGDAADDPAIWVHPHDSEKSKIIGTNKEEGISVYDLKGKLLYSYNFGKLNNVDVRYGFPIGGKKIDIAAASNRSTNTIDIFAINSKTGELENITGSPIKSNMKEVYGFSLYHSQKTGVFYALVVGKDGTFEQYELFDNGKGKIEGKKVRELKLSSQSEGIVADDEYGTMYIGEEDVAIWKFNAEPDGGKQPITKVDSADGNHLTADIEGLTIYYGKNGTGYLIASSQGNNTYAVYDRKGNNEYIGNFAIVDGKNTDGTSDTDGIDVMSFGLGKKYPNGIFLAQDGENIERGKIVNQNFKMVDWKKIAKGFDSKLQIENNVDPRKLKFRQNS

SEQ ID NO: 96 - protein, 391 aa, Saccharicrinis fermentans
MQFINLTTLFIAAASILSSCQPKGNQNNNSEAERDNTAILEKNKEARDDSLKLTEAYALQAQFSNIVSADYETTPVKSNLGDDAADDPAIWINKIHPEKSMIIGTNKKAGLNVYDLQGNELQFIPIGKMNNADVSYNFAYKGQQVDLLAGSNRTSQSIDVLLIDGANQKIIEKPLCSIPSSVDDVYGLCMYFDAVANKHYVFVNGKNGKIEQWLLKNDNDSIKGELARSFWVSSQPEGMVVDHVTNTLFVGVEEDAIYKFKAQAQADTSSIRLTASCNANNAHISYDIEGLTIYRISDTKGYLLASIQGNFSYAIFDLSEKNNYITSFIIKDGVFDGVEETDGIDANASLMGPKFPKGMLVVQDGFNKDKKENQNQNFKIISFERILQFLQ

SEQ ID NO: 97 - protein, 403 aa, Asticcacaulis benevestitus
MTPSRILTFATLFACLAVVTPCSTASAKENTFTLERVVILMRHGIKRPNNDPPLPKRFTDQAWPVWSVPPAGLTPHGEQAIARIADFDRLTYAGLLGSDCPPAGAVRVIADTDQRTIRTAEVYASTVFKGCDVRVEHAGEGHADAHFSPFNEDVASPPLDRKAFMDEALTSGGMAAIDKAHHVDYALLSEVLNLKDLPGCQIDKVCSLSDMPSVLDVSGRDPKVSGALKTASSLSQILMLEYANGFPMDEVGWGKVSESQITALSALHAEEFRLIARPKAVATYASGGLLKDIEAALFGPDASPYTLLVGHDGNIAYVGGALGLHWQAQGFAADDPPPGGALIFELWRDGAGEAFVRVRFRSSSLEGMRNLTHLKRAASTRIPMPLCDVKAECSAATFKALIP

SEQ ID NO: 98 - protein, 405 aa, Amycolatopsis orientalis
MRIAALLPLLLAASLLTGVPAEASQNPSPVAQTRAFVDDAGADPANADADDPAIWVHPENPSASVVLGTLKEGGLAAFDLKARQLQHLAVPAGGRFNNVDVVGDLAVVSDRGRDRVRVYRIDPAGAAAGSRVLRDVTDPAAAPVFSASESEVDEQRTAYGLAAGRDPRTGTRWVAVTRRHETRVALLRLVDKPDGTVGTAPIGTIDLPASFRLPNGRTWSPCGEPDERPQLEGSVLDVGHRVLYTAQEDVGIWRIPLGEEGFGRPALIDKVRSFGVPQRFDEATEECVADGPDPGFGGKWLTADAEGLTLADGKLLASSQGDSRFVVYDRAGTPRRDFRIVAGRGTDSVEHSDGAAITTRSLGPLFPHGLLVVHDGERRPAATGPSGEELATTGFAFVRLEAVVR

SEQ ID NO: 99 - protein, 408 aa, Asticcacaulis benevestitus
MTLKHWLRFALTFSGACLLAIQASAQPTKVERVVMLMRHGVRSPITGEAPLDTQTGAPWPVWSVPPETITPHGAEALKALGNVDRAWLSARGILPAKACPDLAKTIVWTNTSPRTIATGQAYVQGLAPGCALHVGHLPEDQIDPLFEPTRAPPPWFDAQRAVTSINAYTGGMPVLVQRHDATLGQLEHVLDCGASPCSPQRAPRLAVTADNRGLVFEGPVRDASGTAEVLMLEYLEGFPLKEVGWGRANPATLKTVGEVHAALFDVFSRPPYMMAFQTEPTARRIIDDFSRPDAPDFDMLVGHDTNVAALAALLGVTVEAPGFAVNDPSPGGALVLALIRDAQGRAFVRVYYRSQSADDIRAARDHATWKSLTMNACKQGPQHMCPLPDFVALLKAGTAEARAPLVAR

SEQ ID NO: 100 - protein, 423 aa, Acidobacteriaceae sp.
MLFRFVLPAVLLLPAFCSFAQTPTDDVRAVVIVARHGVRAPIENETRSSVFNAQRWPAWPSAPGDLTPHGIAALKRMGEFYRQRYATLLHDCNSVAVESTNTSRTIGSAKAVMSVVVPDCDVRVMADLTADPPSIDKAKLADATHGRMADQPEWFTHAFARPLEEMHDVLAKCAGCKQVPDFRTSMLDPSAHSENMMQAQGEKAGALVPRDPRKENAAALGADFSENFLLQYAEGMPMDQVGWGRVDRARLDDLMEMNTRYHDFILRTPYYAQQAAGAMAQKISDWLSMAAILNIPVRPGRPDVPAGGRESVLYLSVHDANLTWLGGLLRIAWLVSDETLNATPPGSALVFELHHNRQSNADTVQVFFIAQTLDQIRNLTDLTRAEKPSISPIYVPGCSGPGPGYACSMEAFVRVTKEATKVP

SEQ ID NO: 101 - protein, 426 aa, Bacteroides sp.
MAENPLDEIRRDPERAGGVYYVTDLSHPVTPAPKGYTPFYINGYFRHGARQIDDEVTYPAIYGVLEKAHATNNLTDFGKALYERLEPFKKNVFYKEGDLTQIGYRQTREIGRRMVQNYPEVFEGHPYLKTNATNVLRVAATMQSVNSGILSLRPGLEWAEIDNSRSFLTTLNPYGNVCPGRSPLDKYILGKENCWYKKYRSYIDEKLDVDAFFRRLFIDVTQVESEYDKYDLIHRFWLMASLMQCLDRQVPIWDIFTEEEILVWAEIENYKYFAQKGPEPVSHGRSWGLASRTLRHLLDESAEDLVRKRHGINLNFGHDGVLMAILTNLQAGTWAREASNSKEALQSWKYWDIPMGANLQMIFYQSEGNPDVLVKFMLNEKDLRLPLEAVEASYYKWNEVYKFYIEHCDKVEKSLAETLKLSYEDF

SEQ ID NO: 102 - protein, 432 aa, Barnesiella intestinihominis
MKKRTLTAGTILLCLSAISFGQNSIKDFLHQNPQFFGSTASVYYCNDTTDTPAPKDFIPFHIDHIARHGSRTHDSKSMVPNLYKLMNKADSLNLLTREGKLLRNQIDTIYHLMNHRWGDLTPLGARQHRDMARRMYHRFRPAFTPQDGKVTLVAQSTTVPRSMASMAAFVAEMRGYTPTAEFSMDPSNGYDNTLRFFKGKEYQQYLSKGSWKKILRAYQEKHTPTRLIDRIFKKGWEQIIPDPITFMTHLYALTIILPNTDYDISLYPWFTEEEKFDLWSANNLSQYLRKANSIPGKGLPVAIAKPLLKDMLATSQAAIDGNGVEANLRFAHGENTIPLLALLGIENAAVVEADPEKVTEVWQDFKYNPMATNIQWILYKNTDGKILVKVLFNEQETKLPIDSEYAPYYDWKLFQKYCEKQMAKYPDTTPAL

SEQ ID NO: 103 - protein, 433 aa, Endozoicomonas elysicola
MKLQTLLGHCLKKVMAFDYCNNSLKSYLMHGRTSGCFHICSLPESGFKKLFLIFHTVFAMCFLSVGVDATGIGFEMETPADYLISLVRHGDRSPRDLGDMAQYWPMGPGQLTAGGLEQEYLLGKKIRQHYFSESLPDSWSPKISQHYAKGLDRTIQSASALLQGVYPGQPRNTGLPGGIQVPPVYASPLASDDLFSAQRLCPGYLHRVQALEKSADWLRKKEQYRDQLSSWFELSEGSGAGDLYSLIPLIDQISIHRMHRRPMPKGITIQEAIELEELLNWVVSRIIANYEIAQLIGAPLAKAMIRDFKRVQQCLEDKGSCNSCQRWTLYSASDSNLLAIMTMLGAPSDRIVDYATHFGVQLNWNEGRPEIALSLNHEPFSVPGCVGHCSLDQWLVLLEQSLPDDWDYLCARDRGGFAPYPEPYVPSGSVASR

SEQ ID NO: 104 - protein, 440 aa, Asaia astilbes
MLMIGSPVNALAALWGFALSSLLLCGVATPARAVPLSAAPPSADAVLERVVLVARHGIRSPTHDPAALAHETGIAWPEWPVSPGQLTEHGRATLSVMMRDIGRHYDLGCSTHKESCLTQTRPVIWADSADDRTRESGEIMAASLAPDLHLVSRSLGAKVKDPLFGGPPQDFFVREAKRLYKDALTAQKDDMRSRPESVKAGLAAMQLLLAPQGCVKDDGPCLSGPITVTSKNGKPVLEGGPVLGASLAENLLLLHVQGLDHKKAGWTTAIDPAFLTRALAVHDYLSDLTRKRGKLVEEKSRALAGVIDAFLQGRDAQLPNGDSIGPQTRFLAFAGHDTTLDALAARYGLSWSFKDQPTEQLRTRRWLSRDGESQAARSFIALSFSTNPWLHCLKEGGSMKPMEGFRPWCRSRAERAGNSRLSCRREGMMRCTQLSSWRSR

SEQ ID NO: 105 - protein, 440 aa, Alistipes timonensis
MKRPLLILSACLCALAAAVSCSSGLRTADGESVPDRVMGVYLPYPETVAPSPGAPEGYETFYISHYGRHGSRYLLYDSQYAFVRDVLSRAAADGKLTPSGQKALADFLEAYPQFEGRAGMLTRIGAAQHRAIARRMAERHPSAFAPEAAVRASTSATARTQESMEAFCAALQTCRPSLRITCAEDAALNPYSAGSGIPTEYDLRVKSPEAEWRPDFETFCRQQIDAERFAARIFTDTDYAAGLCDLTDLERGAFYLAVHFRGCGIAADWLRLFTLDELCTLAACDAYTFYMEKGPAAETSDRTWALSAHILDKMLTDAEQDIADGTAANLRFGHDGCIMALLTLMGADGWTATAATQEEIARAWDVSQIPMACNLQWIFYRPVSGEGEPLVRTLLNERPLRLPVTDPEGMCAWSDLKRYLAERCDTAFAVLNKDRTPNNL

SEQ ID NO: 106 - protein, 442 aa, unidentified
MKLLNKTTFLIGLGLVFQTMTISAQTLTDRVYEDPTTSANIYHVYKQREGKLTPAPKGYQPFYISHYGRHGSRYLTGDYYFRPAMAMLEAEKEKGNLTEVGENLYSDIKKLAAAHEGMYGELSPRGAREHRAISGRMYNRFTKVFKSKTRNKVNCISSVVPRCIISMANFTTALNDNRPELEFSYTTGDKYFELLAHNYDTDEIYKTIGHITDSLRKATCHYDKFYGKIFKNPTVAVDSMKSPYSLIGSVYSSACICECLDFLNVDLFKYFDKDELAQQAVVRNNRVYGDLGNSIECGDVVAASARFLLQDFVDKADAALQNGSDVAADLRFGHDSGIQPFFCILGIEGHDKQLHIADADKCWFSDLTVCMGTNLQMIFYKNKKGQVLVKLLYNEAEAMIPAVQAYSGPYYRWEDLRSYFVDRIAAAKAFAATLPPPQPREE

SEQ ID NO: 107 - protein, 444 aa, unidentified
MTLRKTLSAAVVTMLTAFALTATAQTTADADISAKEAISKDNNLAGCMYRAYPGPTKQLTPAPEGKRPFYISHYGRHGSRWLIGRDDYKYLVEVLQKADSAKTLTKYGQELLKKLRVINDHSRKRLGELTSLGAKQHREIGGRMADRFPEVFQGGVRIEARSSTVVRCILSMENELLEMMRRYNNLRIWSDASDRDMWFMAGGRENLGDVKGKKAAQELEWKWEQTKMPLFKSMFTKIFNDTAYVNKMVNKDKLVYRLYSIAVNMQSLDERKEISLYDLFDNDQLYACYEKENAGWFINYGSNLWTQGKMPYSQRFLLRDIITKADSCIALPKPGATLRFGHDTMICPMVCLLGIGGRDKMLDDTDFDNIAEKWRMYEFVPMAANIQFIFYRSSPSDGDVLVKVLLNEDEVAVTEDLKTDIFPYYHWKDFKEYFLKKLDNFQLR

SEQ ID NO: 108 - protein, 463 aa, Desulfovibrio piger
MLPSRKEILMRTWTCLAGLLALLLGLMPCPVSAAPSAYHLSKVVMLTRHGVAAPADDNQLPRITGKAWPQWPVGPGQISPRGAQLLTAQWASLRALYLQADLLPAQKTESRVFVRADNTPRSRGTAEALLQGLTPGTLPAYAVVNLDPDPLFEPVQAGLAIFDPAETALAILDHINGDFSQFWESLGEPMSLLDQLAGPLSAEACNSINLPGGCRLSDMVPTISIMDMGRRVEIRGGLGLGSTLVDLLVQEYAQWPQQEAAWGQANAAALRKLLPIRSEIFNALHRTPLIAGIYGGPLLRDMALALYSQHADPRLNEAKCAVFVGNDNNLAAIGALLGIDWQASGFPPNALPPGTILAMELWEGPGNDSEVRFRVFMQSLDFMHAPLSVTTASGYQTAPESYGPALLEAHVTLDGQKDGPVMSRELFEQRVRGALEGRGLPRITFLPEMVSPQAPAPSLPVQP

SEQ ID NO: 109 - protein, 488 aa, Chitinibacter sp.
MRHLFCLSVLSALLTACGGDITDPAATPAPTATPVATATPAPTVQPYTVSAVSFDAETENVLSAAGDVDDPAIWVHPSQSAKSLVVTAIKDGGMRVYDLQGQLVQSIGPGALNNDSKGKSRYNNVDIVYGFKLDDNSTVDLAIATDRGQDLIRVWKIDPNNSTTPLSDITSATPLRLFPTVPTEGSESDASKDAAIAVSKQNTGYGITHFSDKASGKHFVLVNQRKQARIRQFELIAEAGGTVNVVAVAGRDWRFPYSWRGQNLRETNDLDATRDWSPQFEGMVVDQRNGMLYAGQEDVGIWRVDLKTGQADTQPVYETRGSSRETYTTGTPATVVKHSFFNPESKISRDLEGLSIYYGPNGTGYLIASSQGGAHGGAPNVSDAPYDDSFAVFALNGNAKPVLKQGFRLSQGPNGNTEGVQECDGADVVALQLPGYPFGLLVTQDGYNDDLNNLDGVTAQTNLKFTSWEKIATALKLEKYNNFDPRKL

SEQ ID NO: 110 - protein, 510 aa, Caulobacter sp.
MIHRRQRLAAASALALTLGAVAGQTFAQAVAPVGATVPTAEGGANAAALLYDRADPGKSVIAATGELGGLEFHDLDGARKSALPGGETYGVDVRDDVLAVLDRKDGRIRLSRYDFATGQASAIDARPLMLGYAGEGLCLHRSARDGSLYAFALGGDGQLDQWLLFPTADGKLDGRIVRRLHLSSEAKYCVADDASGMLYVAQQAVGVWRYDADPEAEAVATVVDINRLGHIAGEVGGLAILNGGAQGDYLVAANADAGDYNVYDRNADDRFLGRFRIQANGADAIEGPAGLFGVRAPLGADLPAGALLVTDDRKVGANTKLLSWRDVAVALKVSPGQVTPPAAPSRLALVKPTMETRPVEHGGDAADDPAIFVHPTDPARSVIVATDKKAGLYVYDLAGKPLQFLPDGKMNNVDLRGGFKLGGRTTTLVVASDRTHKSIALYTIDPDTRMLTNVADGVQATGLSDPYGCACTRAARVRPTCSSAIPTAACASGNWSPPRPARSAPRRSAT

SEQ ID NO: 111 - protein, 540 aa, Butyrivibrio sp.
MKKTVITACSLVLAAVIGSTGCGKISLKNPNMKNSETNSELKTEDNTEVKTETNTETNTETNTGATTEAKTEAKTEETTEAKTETTTEAKAETTAEDNTEANSEATSEVNAESNTETETNTGSTQNIDEEANAGGRIIDYDGYKLDQVVILSRHNIRSPLSGGDSLLGKITPHEWFNWTSAPSELSLRGGVLETQMGQYFRQWAEEEELIPTNYHPDNGQVRIYANSKQRTIATAQYFTAGMFPTAGLPVEYHMEFDKMDPVFTPQLTFWTDEYEKDAKDQIYEYYADAMKSLNDNYELLSDVIDLEESNGFKDQSITAFTAGDEEFSLKEGAEPGVSGSLKTACSVSDALVLQYYEESDTQKAGFGQELTYEQWKSISEIKDVYGDVLFTAPMIAVNVAYPLVSEIYDEMNTDGRVFTFLCGHDSNVGSVLAAIGAEDYELPNAIEKKTPIGCKIVFTKWSKGSEVFWSADLVYQTPSQLRNMPILDTDSNSPEIFHISFKNIKQNEDGLYKADEFMKIFLDAMDSYESLYQEYVEKAA

SEQ ID NO: 112 - protein, 549 aa, Desulfatirhabdium butyrativorans
MKPKRCFLMTVCALLILISGPAMAQPQAPVLSVTTRGWVYLSWTEVSGATGYTLSYAQIPYTGSSAIGTVDMGTQTSLSGALAADTAFYVAVQSRDNTGISEYSNVVSIAGDSTTLKQIIIFGRHSIRSATSDPASLAQYAVDAYPEFTGVPAGYLTPRGQQAATLLGSYFHEYLLLEGLLTGEAQTDISRSYFRANSIQRSNVTAAKFGSGLIPGATIPVHSYKIADGASSAEPDPVFDPILTKVATVDPARALGEVQGIFNSGAAISSAYSGELSLIRSALYPKGTQPTPGAAHGSTDPTSQTSHPITLKASTSTLYTGGVIDLGGLNLTNSAADPFVMQYTDNFPLNDVAWGRLSLDAVSQQSRIINLIFNIQILPPYLNQVQSSNAAQHVLRTMEQAVTGQNISGAFGDAQTRVHVIVSSDGYVIGLAGLLHLHWLLPGYQPDFCPPGGALVFEVRQVNGTGEHVVRVYYTAQSFDQLRNLTPLSLANPPQTMNLLVPGCSRSATNLDVPFSAFQQVLQNAIGMEYVQDPSKETPPGVLTGVPLE

SEQ ID NO: 113 - protein, 556 aa, Cystobacter fuscus
MFQGVLLTELLLGARGCSSTMRCPRRLGAASTLEEFMSAKRRVLGSASLALVLVPLASLAEPPVIPPRAETPVLHRYDEAPRTPDSDDPAIWIHPSRPERGLVIGVLKEAGLQVYDLSGRVVQTVLPANRPAISAGDPMAPGPRPEAATSACPESESGETFGRFNNVDIQYGFPLRGADGRVRKVDLAVVTDRGCDRLRIYAIDPGRAGGPLFDVTARAARRVFPERFVRPSPFQPTGEPEGVRPNPLDDQNTAYGLALYRDPSNRFHAFVTQRSRAVVAWLELYEAGTEQVGYREVREFRFDSRFSLPRPGGGGALAWSPCREEPAEDPQFEGLVVDQQEGILYAAQEVVGLWKIPLSVSLPRVVDVPRGRLIEPVKSFGAPYWAVPDDGEYACELEAPAPAPEGTIAVPGNPAVGGKHLEADAEGLALYYAGDEKGYLIVSSQGDDTFHLYDREGGWTRERGNRHLGTFQVEGVGETDGHDVVNVPMGAGFPRGLVVLQTGKAPPPPSTEPVNGYAYDGSTRFKLVRWDDIAEAVPPGFKVDTDDYHPRDPHDD

SEQ ID NO: 114 - protein, 636 aa, unidentified
MMIRVLSKLLLPCLLCGLLTACATSPLKATDKLALPPPSAAWKNISAITKKGQPLWLITSETTGLSLIDSSNNRLLALQGHFELLDVRPAHDNEFWALTVDSQSNAPQLLLMQTHPEIRLRERHTLFNGDFQIDGLCLYKDAQSHLMAYFLDGNGSGELRWLVDGSSGKVVDTSITMLNLPPNSEHCTVHDASNSLFISEAEIGIWRYPANPEIENSRILVDRTFDGGKIDGEVMHLTTTRNALYAATEQRLNIYSPAQWQLQSSVPFPPGVTVEGIAASQSQLAVLDEAQQALTLMPLSQPTSYRSSEKHILPVVKANAQTTAMDRRGDAADDPAIWIHPQEPEKSLILGTNKKWGLLVYNLQGEAVRSIATGHINNVDVRQNLLFANEPIDIAVASNRSNNAIALYKIHPETGDVNSWASLPTDLNDVYGICLFTPTPSDIYIIINDKSGRVNQYKLGSRNRKPHATLTHSVQLKSQPEGCVADDTRRELFIGEEDKGVWRFNLTNDKLVEPELIATTNGHLVADVEGIDIVKNEFGNYLVVSSQGDHSYAVFENKAPYEFLGKFRIAPNATLKIDGTSETDGLAVTGMPLGNNYPAGLMVVQDGHNLMPNAPQNFKLISWKDVLNALKIPTVE

SEQ ID NO: 115 - protein, 644 aa, Microbulbifer agarilyticus
MALKTLPKLFSILAVAALCSACGPQIGNDAAKPKGIASEHQLALTDVVSSQLAPLALGGVEYLLLASEKRGLVLVDKEGSEKLALDGGTVERFALHPLEQDRWLIAVYDEDNGELQLRLLDVEEGSPRIRYLAAMPTNAPQVAMCFSSQAGRTHLFAIDETGLGHEYVVHPREQAWTFTGLRPLYFGEQVSSCVVDDRRGKLLVAQPPLGIWSLNADAETDEARQVFIAASALPDGEFGGLWLDEVNGNLWLTAAEKVMAFDINDPAKGPLFVEALADIEPVSAAVQGEALLALEEEGDQVHRFAVSLPQPPAEVQAFRGPVEIPRVRASGQTAPVASGGDAADDPAIWVNPAKPSASLIFGTDKKSGLSVYDLNGKLVEHFEVGRVNNVDLRPMQHGKFVAIAAATNRTDPGVSLFGITAAGAVEYLGLRNLDMEDPYGLCVYRKGADLMTWVSDKEGAVQLLQIVPGTGNVDWSLRKLATLEVASQVEGCVVDDEMQMLFFGEEDGGIWRLDIAAYLAGEAKPQLIAPVDGERLAADVEGMGFYHAADKSYLVVSSQGNNSYALFNRDGSEFVGHFKVDINLDKGLDGSSETDGLEVSSAALGAQYPQGLLVVQDGRNRMPSASQNFKLVSWADIAETLQLP

SEQ ID NO: 116 - protein, 655 aa, Acinetobacter nectaris
MNIIFLKKTCFVSIVLLGLMGCGQKTMPVPEKSVISKDGTIQFESISINGTKDVFKVQSAKLLSIPYWSEAHLIQTSKTQGLQILNRQNHVLQSIDGAFGDIDYRIKDNLLFIQSVDLKQQRPTLLIFDMKSKQWQPSILLEKTKFKIAVCLYQDQSQQLYSFVIGGQGSAQQWAVNTTPNAKQPMQLVRNLNIPIGSKSCVVEDQQERLLINEEGIGIWEYLTDAEQPFKRKIVDMVKPYGHIEGSPGAFTQVDNMLFVVDTQQPFIYKYIHDGKNWQISDQLNTAELKKPEQLSAKREGEKVQLLLNDNHKLKIASLPYTTAHQDKKKDEHFQFVQAKVQTTPVPNVGDAADDPAIWYNEEIPTASRILGTDKQGGLQVYSLDGNEQQYLAVGRLNNVDIRPNFNWDGQRVDLAVATNRDHNSLHLFAIEKKTGHVSEIGQAQTSLKDIYGLCLYQNKQGDIYAIPNDKDGTFIQYHLSALKNKVNAKEIQKFSVKTQPEGCVVDDKNDQIFLGEEDRAVWQKSLLQNNSNLQEVATVGKNQIQDDIEGMGLYHGKKQSYLVVSSQGNNSYAVMEAHAPYRYRGSFKIVMNSVEQIDAVSETDGLDVTSHNLGGVWEKGMLVVQDGHKVMPEDHQNFKYVPWSEISEKLNLEE

SEQ ID NO: 117 - protein, 669 aa, Catenovulum agarivorans
MQYFHQLTHNKLAKVSVVITAAVFALAACQSSIETTAHSAVQALAKAPQQIFQHDKLNGQKVIQLNQDTWVANSENFGLMLINQQGKMVQNYAGNFESLDIRQLDNKTWFVSSVDKEAGQFLLFKVVLAANAQTHKITLLNTIKPEASVIDNQCLAYQNGQLSSFWLTAQQQVEQRIVYLNQPNAQPSNVLVRSFSAPPGASACVVDDVKQQVYVAEESAGVWAYPTNPEKELSRTPIALVAPFGQIDGEIKDISLLIDGSLLVALPEAEQIQHYQTDNSSHLAQVYSVAGFTIESIDAQQTADNKWLANFYDDESGAYYSIELPLMVADKSIAKQGASQLTADMQTAPVARFGDAADDPEVWLNPNNGADSLILTTDKRYGLNVYNLAGELVETIASGRINNIDISYGAEFNGDKFDFATASNRTYNSITMYKISHTGVISELANLPTSLPDVYGLCQYQSPITGDYFVFINDESGIYHQYQLDFSTNQVTGKLVREFKVASQPEGCVADAQTQTLYLGEEDWGIWTIGAEPDAGTRLAPFYQVDGDVLVDDVEGLSLYLPNKSAAKNRSEKYLVASSQGDDSYVVFDLAKNASRGHVVAKFNIVADVQKGIDGASETDGLAVIAAPLGDLYPLGALVVQDGRNIMPVQPQNFKLVDWRKISGLIIKQ

SEQ ID NO: 118 - protein, 736 aa, Corynebacterium timonense
MRKFSGAFVISLAAAVIAQNASVSSAQVADYSAEFSARTTPSFTGSNTSYANSNEASKLSTELQEAPDGFEPSYSALFARHGSRTSSDSGYYDRLINGLEAAGRVDFFGGAPTRTELGKEIEDHLRQLRQLHDQIGGGNLTGLGREESRELADSFAERNRELLSDSEAKLRLEASTKGRSIETGEIFAQQLIAHRFVVAQIPDRATQNKEVLQNQDVPESPAQQRREAYLQTDPGYQAVKGIPENWTAEVQPYANAVFEQLQGSRKPSFYGYCGGRARDLFEARAFSELMSVEREAAGVGPFPAIPGAEDMYRSVQQCTAIDEFYKRGVGLEGQDHSNANGLPLLNQIFAGIDASENNEEVGQLLFSHNEILTPFNAVLAIPEIGANQKFRPGELFNYETYPVLNQAQSDPMMGNIEWNVFKHENGTRLVRMQHNERAVHFGRSCKPINEEFDYFYLYEDLKACLPDAGQSIPSEDELSKARKLLQEALSEEELIQIVSVIENEDGSTTLKLSNGQVVTVPPNRQIVGTEETGSAVIIKLSDGSVIEIAKGPKGEPGEQGPKGEPGEQGPKGEPGEQGPKGEPGEQGPKGEPGEQGPKGEPGEQGPKGEQGEPSRIVAQGVDGEGNTVLTFSDGTVVTIPSGALSIERQYLDSEGNTVVEFSNGQKIVIASPAPSKSSSGSSWSDSSSLTAVGSSAFAAGGVVVAFVLAIAAVVGGLLGIIPLAPPIQSLIDLVQR

SEQ ID NO: 119 - protein, 738 aa, Alteromonas macleodii
MDKQKITRIYPTMGKTFFLTGMLATLFSCATAGKASHQPQADIQEPVLKLKQLANGFSQTLYYRSDNQQTAYKDTHANEQETPKALNKSYKDTHANDNEKLAPALNAQTNDDTHTIELQLNEHQGISISNSQGKARFIAGSFVLASAEIAEFENKQWVSMVVFNNNTQHIEAYRLAPDTLDIQQQLTQSAKGSEAICAAVTAQGAPHIVNIDATGTLNQFEIHDNQFLPLRNFAIGPGMKSCSLDMLTHSIYLADEFAGVWKVNADIESELEKELFFHNANIAIEGVSTLSSNTSLFKGSLLGWVSPNKSGVWLQSQCQTEFITLQYDLAGVPQTIEPEFIHLAFYEYEKGPRISVIVDDDASGNFFSAELSSELSAAYFNHGCLSKDEEPIANAGSRSPKYALARVLPSAETQPVKNYGDAADDPAIWVNAQSPSQSRILGTDKKGALNTYDLLGHQQQSLAVGRVNNVDVGYNVAIANINNSGETTFTDIAIASNRSHNSLSVFEIDEFGVLAHLGEIDTTLTDIYGMCLFVENGVTQVFANDTSGLFERYEVSFNKNKKAKGTLTQSFSLPSQPEGCVVDTHTNTAYFGEEGAGIWSMDINSTKHEPAFVSAIAPPVEADVEGLALFTVDAQTYLIASSQGNNSYAIYNINSKNADKLTLMGLIRITADMNNSIDGVSETDGLEATNANLGGVYSEGLWVVQDGRNVMPSETQNFKLVSGARLKEAIRNLVSTKH

SEQ ID NO: 120 - protein, 791 aa, Clostridium sp.
MKKLFKILFSIFIAIILSFNFITYYPKASNNLELVQDSTNITKIPKNFRKSTDVLNLDEGEKINLTGLKELNISGSAQFSKEGLSLIKKSIGDKYNITVVDLREESHGFINGIPISFKNKNNNANEGLSLNEILNDEAKRLNSIKINEPITFSNTNISVIPKEVYSEETLTKNNNLNYIRIPVTDGGLPSTDMVNRFKETVINNKGNNNWFHFHCKAGIGRTTTFMIMYDIVKNCNEVSLEDIITRQVKLFEMSENDSKGFYSGDRYKFLSDFYNECKGNSKNIFNKNNNKNDYYIKNNIMPKTLYVIKDNEMTKEEQTMVATLQGNVATKSKEQIYILSNYEPSYETWLNDLKENYNVKYKFIKDPYELLSKFKDYIKGYILYNNSNKYSINNGCSLASLENGIVIDELIEEKVKEIGVKNLIKDCRDTDKYWAFNNLWNKGLNHSTVIQLSPDKTMALRDYAIVSKSLVFYEEEINNKEFREKIFSSMDDVSRVFGWGPDEHTNVSIASKFGVDIVASDWSYNLSVLSSYPTRDQKQNLSKESVKDNAHYVTFIMSDGDNQQWFLGSNFNSNNWYGSKYRGKFNLGWSISPSIYYLAPTVFNKYYESASNIRYKDYYLAPPSGNGYMYPSKFPEEKLNNYTKRLSNYMKKVDQNYVLILDDESFYRTDLWDKYTCHNNIDGLIYLDYKKNNNYNGEIVWSNNKPVVSCRDLMWKGLEEEDTLIENINSRVKEGYTDIKNPKSYTCVYVHVWSKNMDDLNLVIEELNKNSKVEVVTPYTFMKLIKENIKK

SEQ ID NO: 121 - protein, 821 aa, Clostridium beijerinckii
MNKNFKRITILTLLIVSIFSFAVNNNAIAQVPSDKDNGVHLILDSVNYNSVLPKHFRKTTDLAVVKDNKDLDLKGLDKLNISGSQQFSGNNIDLLTKAIDTSLPMTVIDLRQECHGFINGFAVSWADARNNANVGLTRDQVIAKEKEDLKSIKLNEPITFYNNSKQTMDVKTVQSEEELTKSKKLGYERVTVRDGGIPTDDMVDYFMEFIKNKPKDSWLHFHCKEGIGRTSTFMIMYDMINNYKDVNADGIIKRQLALANFEDTTLKSFYNNERMGFLNKFYDYCKANGDSFDTKWSEWNSKASSIDETDKSNAIQVSNVNSVYMKNNIVPKSLYAISLDSMTPGERTMITSLQGLVNNHCSFQIYTLNSSQPDYKIWLEDLKSNHNVPYEMISDPWELLKIYKDYIKGYVLYNGETKDPSINNACSLAGVKDAIVIDEKLEPKIKELGIESKGDCRSTDENWAYDNLWNKGLNHSMVMELSPDRIDALRDYAIMTKSLIFYEGSVDKTALRDKIFSSVDDNATCLGWGPDEFINVSTASKYGVSMVAADWSYNLTTLSAFKEPHINKNSSLKVPKEENVHYVTFIMSDGDNQQWNLGTNYGSSKWFGYENRDKLSLGWSMTPALYYLSPTVFDLYNKSISNEDPRNNFIVAPSGNGYIYPSKFPRNKLGSYVDLLNNYMKRVNENCVSIIDDSAFHDIELWNEFTKKSNIKGLFYIDYHRHDNYKGEILWSNNKPLVSCRDLLWNSLEDEEELVKRINDRVSLGQTNIHSPEAYSFVYVHVWTKETSNVEKVTKMLKENKSVRVVPPETFMELISKNVKH

SEQ ID NO: 122 - protein, 293 aa, Bdellovibrio bacteriovorus
MKKLLLLPVLFAVAACAEKTVSLTPDKPVSTKIPFFMTRPQPTTPVELVFDNDHATAKPMNYRKNDSLRMSGSATFSPKALKEVSKPVKKNKASLYVFDLRQESHGLINDIPVTWYADRDWANADLNHEEAVRRERRLLGDLRVGEKIGTTTIQSIETEESMIRTGGHQYVRLTVTDHVRPVDSEVDRFIESVRALPENAWVHFHCRAGKGRTTTFMVLYDMLKNAKADSFEAIIKHNTELSNDYDVLTVPADEKDWKYPYQKERAAFVTEFYNYAKAHPNGEGMLWGEWVLR

SEQ ID NO: 123 - protein, 301 aa, Clostridium tyrobutyricum
MKKNRVRIGIFLLFFIFLLGASNVNVLALQKNTADNKLELTVDSHKKAEMPKRFRKTSDKIKTEGKMPNLTGLAELNASGSQQFTPGNINLVKDAIGKDKKISIVDLRQESHGFINHLAVSWKLPDSNKANKGLTREQVIKEEKKLLKSIKLNEPLKIKNKTVIPVKVQSEKQLVKDTGMDYIRVTVTDTERPEDDMVDYFVKKVRKLPEDTWLHFHCKAGMGRTTTFMAMYDMMKNSKKVSLEDIMERQELLGGVKLLKPVGGKESESQKRSDFLRQFYQYTKENNDNFKTSWSQWLNSK

SEQ ID NO: 124 - protein, 302 aa, Acidaminococcus fermentans
MKFWKITTGILLMSQLSLGLAWADAPSIAAIVKPSKGYVWRLDTKNKLQLPRNYRADVENCMSGSAQPSILGLSSLVQELAQQGVKPQQIILVDLRQESHGFVNGQAVSWYGDNNWANVGKADAAIRKDEANRLAKTLGKETSYYKLDKNKQPHFKGKENVAAALTERQAAASFGLGYARFASTDHIWPEPEEVDAFLAWQKTLPKDAWLHFHCQAGKGRTTAYMIMRDIWLNGQKDSLETICARQHALGGQDVLHMTHKEAWRQTIDDNKVYRLKQFYTYVKGLQQGTITGTWTEYLKQNP

SEQ ID NO: 125 - protein, 307 aa, Desulfovibrio alcoholivorans
MTPLCSARFLPRLLACALFLAILGAAKPGPGADGPVLIYDYPPGQALPIHFRLADGGPKGDPASAKLRLSGSSEFDAAGLDNLARTLPGPLTIVDLRQESHGFLNDRPVSWFAPKDEANMGKTPEEAARDEAQRLAALSKAGTAAVTVIEAKSKSGGIKKARTLRVDVVGVASEAQLTAARAVGYLRLFVTDDMAPDAAQVDRFVEYCRSMAPNTWLHVHCHAGDGRTTTFMVLYALLHNPAGRSLEEIAADQAAAGGVDLIGQPRKGWKKPLYMARVAFLERFAAYAAANPGGAPMTFSAWTATHK

SEQ ID NO: 126 - protein, 317 aa, Desulfovibrio frigidus
MSAKIFFPIKTRILDDIFKLILASFVILLIFNSNSFADDSVLILDAPAVENLPRNFRTSAFPFKNSTDSPSRTGLDTLKISGSGQFSADQFDVMRSNLPATVTVVDLRQEFHGNLNGAAVCWFSYRDSGNKGLDSAQVTSGEQEALSLLGAQKEIQLGMNIIKNDDGGFKVDKKYQLKPHTVYSEHELMGARGVGYIRIACTDHLRPPDVAVERFVKFVRNLKSDVWLHFHCRAGKGRTTTFMLMYDMLRNANKLDLGTLAARQKMIGGSGLLGKVSTDKEWKIEIGHERQNFIRKFYDYAKANPNGLPMTWLEWQK

SEQ ID NO: 127 - protein, 319 aa, Clostridium acetobutylicum
MHKKTKLICIGILATAIVTISIYNFDFFKRTPLKEHKVKLVIDAENKNTLPPKFRTTSDTISLHKKGSLNLSGLSDLNASGSGAFSEDELKSIKNKIGNKPIVDIDLRQESHIFVNGIGISWYGKNDDANLNLTSSEVLKDENNKLMRISKDKKVTFDKLSKKKSISNISQLNDVKSVETEEQLAKALGINYSRITVPDHKTPDDAQINSFVSFVKNLPKGTWLHFHCRGGKGRTTTFMAMYDMMINSKKVSFYDIMKRQKLIGGADLLSGQDSLGKSDAEKRVQLLKKFYNYCQNNNDNFKTSWTNYNNKYRIKNLQR

SEQ ID NO: 128 - protein, 324 aa, Allisonella histaminiformans
MKLQWMTTLLLTSCISLAGYCGSGAAEMHSPIPLQQEIPPAPGYFNGRLWRLDASASSPFPSHFRTSASAFDLSLPHKLPLPSRKGLDRLRISGSAQPSGMAIPALARHLKYLASGGPVFLIDLRQESHGFFNGNAVSWYGNRNWANRNKTTEAVCQDEKERIQSACGTQVTVYSLNEEKEPADAEKITVQSASTEEALAARAGLHYVRFPITDHTTPSEATIRQFISFMRSLPPDAWVHFHCEAGMGRTTTMMILWDIYNNPELPLETIVERQHLIGGAQISLYPQEERKGPWKFNESQSRARIIHMFYDTMHSQSASHADRA

SEQ ID NO: 129 - protein, 328 aa, Centipeda periodontii
MHAFKHLLTALLLCTLCIIPYTASATDTNADYRGYVWRIDTAAGNEAELPHNFRTAGSPFQTRTDTAKFGVDPNYTPSREGLDALPLSGSAEFSVPAFHALLKDLHTRAKGSICIVDLRQESHGFMNGNAVSWYGKHDWGNIGRTKYEALCDENMRIRSAQGKDVVLAHLDKKKQPKNPQTIRVITAMTERELVEDAGVRYVRLAVTDHKWADPQTIDNFVDLVKKMPADTWMHFHCQAGKGRTTSFMAMYDMMKNPSVPLKDILYRQYLLGGAYLAYDPTTLHAPTGWEDADYHHKAEMIAKFYDYVQQNHKNDYAIPWSMWLKKNP

SEQ ID NO: 130 - protein, 331 aa, Desulfovibrio magneticus
MPTLPPPCRRLRRSHRTRLAAPWPLAGLLAVALALFLTGPIAVAWAEPEPDVGVLTLDAAAASALPHRFRTCFFPLARPDGATTPSQEGLNGLRISGSSQFSLAGLAIMREQFPPRAVIVDLRRESHGFLGDAAVSWRLPDNQGNPGREAAFVAAAEAGLLAAIDERPDIVVAREAKRGGPTPLTLGPLAAVNEAQAAASLGLGYLRLAVSDHTRPDDAVVERFVRFYRSLPPDVWLHFHCRGGAGRTTTFMTLVDMLRNAPAVAFEDIIARQKALGGSDLAKTTGGSAPGRDALARERLDFLRRFYDYARANPSGAPLGWRAWLAGGAKP

SEQ ID NO: 131 - protein, 397 aa, Burkholderiales bacterium
MKAKICGSLCLLSAAVLLSNAHAETPSDVLQAIPHTEELLNVVVVSRHGVRSPTQSAEKLHGWSDKKWSAWPVAPGLLTSRGFYLVKATWKLNRSRAPFTYGVCPKPEDVQIIADVDERTRKTAEALNEGLYPTCGYKVKVTSSEHSAIFSPLKAKVCRIDYPKELEEKLTQKVVGINEKFAAQMAEISKLTGHEFTGPMRAKVSKYKVGFKGAPYDCSSITEIFALEWGQNPEKKVAWDQLDWNGILHLMPIRVSVFSALNRDMEVARYKGSALANKIIESLDHGPKYTYLIGHDTNLANLGEIFDLNWKLPGRDQNENTPGGYLTFEKWSVNGQPEIRVFYSALSPEQIHAERVTQPTDDFEILPRGTKFDEWKTTYGRRLQSNCIADDKYENRK

SEQ ID NO: 132 - protein, 424 aa, Acinetobacter nectaris
MIKNKLICAILTSITALTGTTVYAATNDYQLEQVVTLVRHGIRPQTNLQELNDATGKDWPKWSVPDGYLTQRGYDGILNQSQYQLKTWQEAGLPISLNQENCSNQNQVFIWAAPDQRTRKTADAIAESISQTCHPEVGVSGYKHDPLFDALKMGSATDQFDEIKKEFKARIVSQDDIQAKYAQSNQHLKATVCAPNGCDFLDKDWKLKLNKNGQPKLNGPADEGSNIGETIRLQYSENFPLSQVAFGHVKNANDVKKLMVLHDAKYKYLNEIPLFAQRGGSILYQQILDALDNSKATEAPLHRPLVVFVGHDTNISEIKTLLQFNWQLPQYLANDIPPGGTLSFEKYKEKSTGQYFVKIGFSARTLDQWRHLTPLSKAQPLHEDVLKYSYCKTTSVGVLCPLNQFLQNAQKQIVRLNIDQPLYK

SEQ ID NO: 133 - protein, 431 aa, Dyella marensis
MRPPGRFARAALLGAALLAPVLAQADDATLRLRVVVLRHGVRAPTKAPDALAPYASRPWETWPVAPGQLTPHGIEGMRALGANYRRTLAADGLWSGACDRMDQLVVIADSTPRNHASGAALVQGLAPACDGTYLALPTDQNNPLFHFGGKDDDKDEDDKPALPVTWPPAVLAQLQRVLLGCEGQACLAKAHADGRKTLLDAADDAARAKALKTAGSLSENLMLEYAQGFPSAQVAWGQGDEATIGRLVTLHNLQFALSKKAMPAAARGGSNLLAHVLATLQQADGEKPDAAALAPASARAVLVVGHDTNLAQLAGLLDVDWHDAAHPDDYPPGGALVFDLWQTHDTYSVTVSTAMPTLDALRRADVARPTALTRKTLRLTPCPVTDHCPLDKLSSWLRGRLDATRIDPALPDMRAWSASSQEAPHEPAVSK

SEQ ID NO: 134 - protein, 432 aa, Escherichia coli
MKAILIPFLSLLIPLTPQSAFAQSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 135 - protein, 436 aa, Anaerovibrio sp.
MQPLNYTIKKCLGLLGPAAISALLLLSWSTWTEAASELTDKGYQLDQIVMFSRHNLRTSITSEDSSAATMMTHPMPKWQVPRGNLTMKGGVNETILGQYFQLYLMDEGFMADSWQPGYGQVDFYANSYERTIATARYFATGLLPTANITIKHTKAVGGSDAVFNKPFAMDTPKLMELAMEYHNEYFQSYKDVFSKCYDSFARVTDFPNSPYAKKHGIDHINTDDLALTFSGKEKNVINYDGTARKAYKLLDPIIMNYYQEADNSKADFGTGLTRKDWENLGAVKTKSIEMVVGNPAVALSHSHDILNIMESDLKNDTLKFAFICGHDTNIFTILTALETEDYSLPETILTKAPLGVKIVFEKRRNPQGDLFIYPYIAYMSDDQQRNSRQLSLHEPPMIYPLNFRGLAKNGDGLYRYEDILTLMDRVNAKYEEYCLH

SEQ ID NO: 136 - protein, 436 aa, Avibacterium paragallinarum
MKKLTLSLNLVSLLSGSLFYPTNLSAAPNVPDNHQAQALQLKKIVIFSRHGIRTPLVGKESILSQATPYRWVKWQDKTGYLTAKGARLEKLFAAYWSEWLQQTGILSSQCPKDDLFIYTNARPRTIDTGVSFAKGAYSHCAIRVNYLGEYNTMDNTFNPVIRSKVDKAFEQKARKAVDELVGEGGFAQLNRHLQSNFNALSQALNQGQSPLCQEKQLCNLDQEANTLTFTQGKEPKTTGALRDGTGAADSFLLQYYEGFPAKDVAWGRIDNQESWKKIVDIKERYNQLLFGTQVMAKEAATPLLTFIQNSFKDYGYQHPYIEKARNAKVVLLVGHDSNVGSLLPLLKVKPYYLPDQLETTPISGKVAFEKWVNAKGEAFMKVEYVYQTIDQLRNGTKLSLTNPPNRITLQLEDCPTNAQGLCKMDDFYNAVKNALK

SEQ ID NO: 137 - protein, 438 aa, Cronobacter sakazakii
MKTLFLHLFLFIFIFMPGVFPLHAQGADKMKLERVVIVSRHGVRAPTKFTPLMQEITPYPWPQWDVPLGWLTTRGGELASELGRYQKRVLLDKGILESHGCPSPEQVAVIADTDQRTRKTGEAFLAGFAPGCQNKVHYQKELDKKDPLFNPVKMGVCSFNVSKTREAILTRAEGNIERYTQRYDSAFRTLEQVLNFSQSAACKSPRQPGCTLTGVLPSELSVSQDNVSLFGAWSLSSTLTEIFLLQEAQGMAEVAWGRIHGEKQWTELLRLHNAQFDLLQRTPDVARTRATPLLDLISRALISNGSTESHYGIKLPVSLLFIAGHDTNIANLSGVFALNWSLPGQPDNTPPGGELVFERWKRVSDNTDWIQISFVYQTLQQMREFKSSSSSSLPHKIVLPLPSCQEKNAEGMCSLKRFNDIVQTIRVPQCAVMAEKDQ

SEQ ID NO: 138 - protein, 451 aa, unidentified
MLGSRNLLTVFLFTFTSLSFAQTNVIAGSKTAYQYNSNQTLTLAPTGYQAFYIDHVGRHGSRYISKSKYEDLAYHILLLADSQHQLTDSGKDLLRQVTILKQLNQNHYGQLTNLGRKDISLISNRMLENNPTVFKGQKIEVISSSSPRAKETAEIFIDSFKAKYPNIHVIQQPDNEQTLLRFFEYSPAYSEYKKSKAVKDAVKSIEYASQTKQMSKQVAKGIFKKSFLIKLKKGLDESEDTLVKTSDFVIAIYQLYQELQAFSPQVLTDNHLDLGRYFSTEQKIWLNTVVTAKNYLQIGPAFDATGIQIKIAAPLLWDMIHSADKAIVDNNVDANLRFAHAETVSPLATLLEIEGSANVTKTLFNYPTIWRADKIIPMGANIQWIFYKSKQTHQPILIKVLLNEREVHLPIKTDSYPYYRWNNVKQFYVNKLNKLGLDENQSAIEMLKNLK

SEQ ID NO: 139 - protein, 544 aa, Corynebacterium singulare
MRIRTLALATAIACTLTPVQALAADDTTYYSTKQHYEPQGSNYSTAPTGFHQIYTSTVNRHGSRGLSGFKYDDLAQQMLEYAKEHDQLTELGEKLIPQVEAMITVNKELAGGPGQEAGYGNLTVVGREELQGIGQRNAQRNAALMESIEEDNLKVKYMSSGADRANDSGWSFGEAWLSHNSKLSDNLVDGMEDGHVAIETRTDLLHAHKDKKSPSYERYSKWKDSETLDSKVQEAYAKPASQTAARSLLNKIFTEDFIAGLEDGTITFVGRDNKGKSVEGIVEAALQFYNLYIIAPALAHEEQTPSEGWIFDQYMDNASSPTFAYLLDVEDYYQKGPAIEGQTVAYDNYEPLLEEMIQGVKDRAEGGDVAAEYRFGHAETIIPLAALLKLPGSEKGTPADELYTWENSEWRGDKVAPMGANIQWDAFQNERGETLVRMLYNEKEIAFHDGCEPIETGSTFYTIDELSDCLPLGATSDHSKARLQEDKTHETAQPSPSDAVSSKAKVWGIVAAVLGALAIAVGAATANAQQIKDILNKFGIHVPF

SEQ ID NO: 140 - DNA, 1149 bp, Bacillus subtilis
  source 1..1149: mol_type = genomic DNA
atgaaggttccaaaaacaatgctgctaagcactgccgcgggtttattgcttagcctgacagcaacctcggtgtcggctcattatgtgaatgaggaacatcttttcaaagtgactgcacacacggagacagatccggtcgcatctggcgatgacgcagcagatgacccggccatttgggttcatgaaaaacacccggaaaaaagcaagctgattacaacaaataagaagtcagggctcgttgtgtatgatttagacggaaaacagcttcattcttatgagtttggcaagctcaataatgtcgatctgcgctatgattttccattgaacggcgaaaaaattgatattgctgccgcatccaatcggtccgaaggaaaaaatacaattgaagtatatgcaatagacggggataaaggaaaattgaaaagcattacagatccgaaacatcctatttctaccaatatttctgaggtttatggattcagcttgtatcacagccagaaaacaggagcattttacgcattagtgacaggcaaacaaggggaatttgagcagtatgaaattgttgatggtggaaagggttatgtaacagggaaaaaagtgcgtgaatttaagttgaattctcagaccgaaggccttgttgcggatgatgagtacggaaacctatacatagcagaggaagatgaggccatctggaaatttaacgctgagcccggcggaggatcaaaggggcaggttgttgaccgtgcgacaggagatcatttgacagctgatattgaaggactgacaatctattatgcaccaaatggcaaaggatatctcatggcttcaagtcaaggaaataacagctatgcaatgtatgaacggcagggggaaaatcgctatgtagccaactttgagattacagatggcgagaagatagacggtactagtgacacggatggtattgatgttctcggtttcggacttggcccaaaatatccgtacgggatttttgtggcgcaggatggcgaaaatatcgataacggacaagccgtcaatcaaaatttcaaaattgtatcgtgggaacaaattgcacagcatctcggcgaaatgcctgatcttcataaacaggtgaacccgaggaagctgaaagaccgttctgacggctag

SEQ ID NO: 141 - DNA, 312 bp, unidentified
  source 1..312: mol_type = genomic DNA
atgaaatttttcaattacattctgtttctgatcatcattaatatcagctatagccaaatcctgttcgtttttcagcacatccgtcacggcgcgcgtgaaccgatttattacgaccgcacgcgcaaggacaagaacgtggattattatggttaccaatggacgggtaaagccgttctgacgccgatcggtaaacgcatgaattatctgctgggtattcgtaatcgcaacaagtataagaatctgctgaatgagatttacgacagtcgtgaaatctttgttagtagtacgggtttcgaccgcaccattaattga

SEQ ID NO: 142 - DNA, 345 bp, unidentified
  source 1..345: mol_type = genomic DNA
atgaaactgggtacgcaaagcgagggttgcgtggtggatgaccgcaccggtatcctgtacgttgccgaagaggatgttggcctgtggcgtattgacccaggcagtacggttccggttcgcattgccgcggccgacggtcgccagatcgttgcggacgccgaaggtgttgccctggcgccggtgggcgaacgtggtggttatgtggttgttagtagccaaggcgacaacgcctacacgctgtatagtctgccagatgagcgttatgccggtcgctttcgcgtggttgacggtccacgcagtgcggaccgtcgccgtccgggccgccatcgtacggatgcccgctga

SEQ ID NO: 143 - DNA, 489 bp, Clostridium colicanis
  source 1..489: mol_type = genomic DNA
atgggtctgaactacagccaagtgatcgcccgcaacaatgcccaactgagtagtatcaaaatcggcgaaccactgacgatcaagaataccaccattgttccgaaagaagtgtatagtgaggagacgctgatcaaggagaataataaccaatacattcgcatcccggttacggacacgaaactgccaaccccggaaatggtggatctgttcgtgagcagtgttaccaatctgccaaagaatagctggctgcattttcactgtaaagaaggtatcggccgcacgaccaccttcatgacgatgtttgacatgatgaagaacgcgaaaacggtgccactgaatgacatcgttctgcgtcaaatcaagctggcgaatctggagaataaagaaaagggcctggagagtacgacccgtatggccttctataacaagttttacgcctatgccaagaatggtgatttcacgaaaaaattcagtgaatttaacaattaa

SEQ ID NO: 144 - DNA, 495 bp, Chlorobaculum limnaeum
  source 1..495: mol_type = genomic DNA
atggcgacgaaggtgcgtgcctttggtgcctggcgcggcgttaaggaaattgaggccgttgcggtggacgatggtaatgcgacggtgtactacagtgacgagcgctttggcatccgtaagtatagtgcggatccagcggccgtggacgccggtaaggaactggccgtgttcggtcagagtggtttcgccgccgatcgtgagggtatcgccgttacccgcaacgcggaaggtggcaccctggtgctggttagtgatcaaagcggtggcgcgctgcgtatctatagtagcaccaaggtggacgagaatcgctatcgttttatcacgagtgtgccatatcgcgccacggaaaccgatggtctggatctgagcacggaaatccgcaccccggaattcccggaaggtgttctggttgccatgagtgatgaccgtacgttccaattttacagtctgggtgaggtgctgaagcgtctgggtgaacgtgtggacggcgaataa

SEQ ID NO: 145 - DNA, 528 bp, Bacillus amyloliquefaciens
  source 1..528: mol_type = genomic DNA
atgaaccatagtaaaaccctgctgctgacggccgccgcgggtctgatgctgacctgtggcgccgttagtagtcaggcgaagcataaactgagcgatccgtatcattttacggtgaatgccgcggccgaaaccgaaccggtggatacggcgggtgatgccgccgatgatccagcgatctggctggacccgaaaaatccgcaaaatagtaagctgatcacgacgaacaaaaagagtggcctggtggtgtatagtctggagggtaagatgctgcatagttatcataccggtaaactgaataacgtggatatccgttacgactttccgctgaatggtaagaaaagtatcctgcgccgccacccaatcggcctgaaagaacgtattccgctgcgcttcaccccactgacgggcaaaacggcccactataaggccctgcgtatccaaacggcccgcctgcatcagcagctgatgaagtataccgtgagcgcctgcaccacggtgaaaaagcaagaaaatattacccgctggtaa

SEQ ID NO: 146 - DNA, 708 bp, Cystobacter fuscus
  source 1..708: mol_type = genomic DNA
atgggtggtgccgccctgcgtgcgctggcggccaatacgccgggtccgctggttgtggttgatctgcgtgaggaaagccacggtttcctgggtgatctgccagtgagttggtatgcgccgcgcaacgtgggcaatcgtggtcgcacgcgtgaagccacgctggccgaggaactgcgcctgctggacagtctgcgtcgtcatgagagtctggcgtttgatggtcaaggcaaagatcgtggtccaccggaaccagttcgtccaatcgcggccttcggtaccgtgtgtacggaagaaagtatttgtacggaagccggtgccggctatgcccgcctgctggttaccgaccatcatggtccggatgcgggtgagctggaccgtttcgttgcctttctggagcgcctgccggatggtgcgtgggtgcattaccactgtcgtggtggccgcggtcgtacgagtacgtttctgctgctgcatgacctgctgcgcaatgcccaccgtctgccgttcagcgttattgcccaccgtcagcgcgttctgagtgacggctatgatctgctggcccacggtgaaccagcagattggaaaacaccattgcgtcgtgcacgtgcagaaattgttccagcatttgcggagttcgcgcgtgagcgcgcggttggtggcagccaacgttttaccgagtggctgggtgcccgtcaagaaatgcgttga

SEQ ID NO: 147 - DNA, 783 bp, Acinetobacter baumannii
  source 1..783: mol_type = genomic DNA
atgaatattctgttcaagatgacggttctggccgcgagtctgctgctggtggcctgtaatgacaatgatgaccaggaggcgcaaaccgccccgagtaccaaccaaagcaaatactatcaaaccaaaacgccatatcagccgcaacaagagattaagaattatgagcaaaccccaaacggctttcagccggtttttaccgaactggttgcccgccatggtagccgcggtctgagtagcctgaaatatgatctggccctgtataacctgtggaagcaagccaaagcggagaatgcgctgaccccactgggtgagcaactgggtgccgacctggaagcgatgatgaaggcgaatattctgctgggttatggcgtggagggtattcgccaatatggttacggcaatgagacgatgacgggcatcctggagcatcgtggcatcgccgatcgcctgctgcaacgcctgccgaccctgctgaacagtcaagccagtatcctggttcaaagtagtggtgtggaccgtgccgttgacagtgccaaattctttacggccgagctgatcaaacaacgcccgccgctgaaagataaaattgtgccgctgagctacaccaatctgagtagtgagagcgtgccgagtgttgaagacggcggcgtggaccgcttcaagctgtatttccacagcctgaatgcggatgaggacctggttcagccgctgagtgccagtcagcaaaagtttatgatgctggcgcgtccgattaagatcctgaagaaaatcattaaaatttga

SEQ ID NO: 148 - DNA, 807 bp, unidentified
  source 1..807: mol_type = genomic DNA
atgcgttgggttaaagcggtgctgacgttcggtctgctgggctgtctgctgaagggcaaaccactggttcccaagccaccgtgtttcgtgaaagacccgaccagcgccaagcgtttccagacgcgtctgcgcgtgctgagtccacgcaatccaaccctgacgaagaacctgcaacgcctgcgtgttattggtagtccgcaaccgagttttaaacagctgaaaatgctgctgcaagaaaatccgcaactgtacggtatcgttgacctgcgtaaagagacccatttcttcatgaaaggtcagagtattagctggggcacgaagggtaattgcgttaatcatcgtacgattcaagaacatctgagtgaacatgcctggacggagctgcaaggtctgaaatatctgcgtctgcacgttccggaccattacccgccgacgatcgaacaaattaacacctttctgcaattctataagagtctgccaccaggcgcggttctgctgattcattgtcgcggtggccgtggccgcacgacgaccttcctgacgctggtggacatcattgaaaatgccaagtgtgacagtttcgataccatcctggcccgccaatggcgtctgggtggcgccgacctgcgcaagccacgtacgggtgttaaaaaacgcccagccgaaacgcgtctgaaactgctgaagctgttttatgcgcgcgtgcgtaaagatgccgagggcagtctgcgcccatttcagcgccaacgccgcaagacgcagctgccagaagccctgagcctgccgccaccgagtgtgctgatctga

SEQ ID NO: 149 - DNA, 843 bp, Clostridium ihumii
  source 1..843: mol_type = genomic DNA
atgagttactttaagaagctgctgctggtgctgctgctgattttcagctttgccttcaatagttttagtgtgctgggctacgatgaagccagtaatatctacacggttctggataatgagaacagtaatagcctgccgaatctgtttcgtaaggacgcgagtctgaatattagtggtagcgcccagttcacggccgcgcaagtggagaatatcatcaataatattgatagtaataaactgtacatcgttgatctgcgccaagaaagtcatggtctgctgaacgaaaatattgcctttatgttttggaatccaaatgtggacctgaatgacggcctgagtagtagcgaggtgctgcgcgtggagagtaaggagattagcaaaatcccgatcagtagtgtgatgcacatctataataagaagctggatctgctgaaaaccgtgaaagttcagagcgtgctgagtgagaagaatctggttaccagcaaaaatatcaattatattcgctttgcggtgaaagacaactacattccaagtgatgacatcgttgatgagttcgtggactttgttaagagcgagggtacgagtagtcatctgcacttccattgtgaagccggcgagggtcgtacgacgacctttatggccatgtaccaaatgatgaataataaagataacctgagtctggacgagattctgcaataccaaatcaaactgggcggtattagtctgattgacatcccaatccgcaaggatttcctgaatacgttctataactacaccaaagcgaacattgacagtaattttgaggtgccgtatagtaaatgggttgaacgtaaagttatctaa

SEQ ID NO: 150 - DNA, 852 bp, Desulfovibrio fructosovorans
  source 1..852: mol_type = genomic DNA
atggagagtgatccgacgctgatcttcgacagtatccgcgaacaaggtctgccaagtcattttcgtagtctggcgggcccgtgggccgttccaattaccacaacaccaccaagtcgtcaaggtttggcagaattgcgtgcaagtggcagtgaacagccgagtagtagtgaaatggccacgacgatcaagggtctggccccggaagtgaccgtggtggatctgcgtcaagaaagtcacgccctgctgggtgagcacccggttagttggtacggcctgcgcaactgggcgaatgacggtaaaagtctgcaggcggtggaatgtgatgaggaaacgcgcattggtgacctgccgccatgtggcgaggccgcggtgagtcgtgtgattagcaaggatccagacggtgccctggccgaagttcgcgtggaagaggttgcgtatgatcgcgcccgtagcgaacaagaggccctgtgcggcctgggtctgggcaccttccgtattgccgtgcgcgaccatagccgtccaagtgatgcggacgttgatcgcttcatccgctttgtgcgcgaactgccaccgggtacgtggctgcacttccattgtcacgccggcgacggtcgcaccacgacctttctgctgctgtacgacatgctgcgtaacgccgccgttctgggtctggaggagctggccgcccgccagcatatgatcggcggtatcgacctgctgcacacgccgcataccggttggaaaggtgccctgtataatgaacgtgcggcctttgtgggtcgtttccaccattacgcgggcacgcgcgatttccgtcgcgtgctgtggacccagtatctggccagcgcctga

SEQ ID NO: 151 - DNA, 858 bp, Clostridium novyi
  source 1..858: mol_type = genomic DNA
atgaagaaaacgaactttagctttacgctgttcatcctgctgtttctgattagtatcccggttaacggcaagctgattagtacggatgcgccggttccgccaaaaccgctggttgtgctggacagcaatagtacgagtccactgccgagctttttccgtattatcccagaactgaatattagtggtagtaagcaattcaccccggcccagctgaagaacatccagaataaaattaacagcaaaaatctgtatatcgtggacctgcgcgaagagagtcacggttttattaatgataatacggcgattagtttctatctgccgcgtaagtacattaatgataattttaatacccatgagatcctgaaaaaagagaagctggacctggatagcattaaaaatgttgaaaatctgaacatttatgaccagctgggtacgctgaaggaaaccctgaaaccagataaaacgctgagtgagaaggagctgaccaaacacaataagatccattatgtgcgcctgccagttattgataactacatcccgagcccagaaattgttgacaagtttatcgaactgattaaaagtaaaccaagcaatagtcacctgcactttcattgcaaggaaggccaaggtcgtacgaccatgtttatggccatgtatgagatgatgcacaacaaagataatctgagcctggacgaaatcctgaaacatcaacaggatattggcggtattattctgacgggcaacccagcgcgcggtacgttcctgaagaatttttacaattatacgattgacaataaaaagaacaatttcgataccagttatagccaatggctgaagaacatcagtagcatgaaataa

SEQ ID NO: 152 - DNA, 876 bp, Clostridium bornimense
  source 1..876: mol_type = genomic DNA
atggccaaaaaaatccatttcaactttatcaaaatttttatcgttctgatcattttcagcctggccctgccaaatctgctggtgaacggtaagagtccaagcagcgacattcacattgtgctggatacgctgaataatacggaaatcccacgcaattttcgtagtacgaataatattaacgttagtaatagcatcaatacgaaaggcatggagaacctgaatatcagtggtagcagtcagtttagtaagacgaacctgccgctgctgatcaaaagcattaatacgaatctgccgatcatcgacattgatctgcgccaagagagtcatggctttgtgaacgaaatgccgatcagtttcaagaacgataagaataacgcgaatctgggcctgagcagtgaggaggttctgctgaaagagattaaagacctgaacaccatcaatctgaacagcaccctgacgttctataacgacaagaataatccggttctggtgaaaagtgttgaaaatgaaagcctggtgattagtaaaaacagcatcggttatctgcgtatcgccgtgacggacagcatgctgccgaataaggatgaggttaataaatttgttacgtttgtgaagaaccaaccgaagaatacccatctgcacttccactgtaaagagggtattggccgcacgaccatctttatgatcctgtatgacatgatgaagaattacaacgacattccaatggaagacatcattagccgtcagatcgcgctggccaaactggacgaaaaagattatatggccttttacgacaaaaatcatacggaattctttaccagtttctataactattgccgtagcagcaaggatctgttcctgagcccgtggcaatga

SEQ ID NO: 153 - DNA, 894 bp, Bacteroides stercorirosoris
  source 1..894: mol_type = genomic DNA
atgttttgcccggtgagcattctgtggggccaggatcgtattcaacaatacgcgggtacggccatgccatatccgatcgtgaaggatagtttcgtgttcttccaagacagtatggtgccgttctacgtgaaccacctgggtcgccatggcgcccgtttcccaaccagcggtaaggcgctgaatcgtgtgaaagagatcctggaactggcccgccgtgagaatcgcctgacgagtggtggcgttaccctgctgagtacgatccagaatctgagcgatacgtttgacggtcaatggggcaaactgagtgtggttggtgaggaggaacagcgcggcattgcgcgccgcatgatcgaacgttatccgcaactgttcagcgatagtgtgaaggttcaagcgattgccacctatgtgccacgctgtatccacagtatggacgccttcctggcgtgtatggtggagtttaatagtagtctgcacattcagcgcaacgaaggtaagcagtataatgatatcctgcgtttctttgacctgaatcaaagttatgttgactacaaagaaaatggcgactggcgcccgatctatgagacgtttgtgcgtcgcaagattagtccagccagcgttatggaaaacttctttctggagagtggtcaggaaaccgataaagaggcggaggagttcgtgatggccctgtttagcattgccgcgatcctgccggatacgggcaccccgattaatctggacggtctgttcacgatcggcgaatgggataattattggcagacccaaaatctgcgccaatatatgagtaaaagtagcagcccagtgggtcgtatgctgccggttgccatcgcgtggctgtgctaccaaaacctgtttattcaactgatgaagtaa

SEQ ID NO: 154 - DNA, 912 bp, Clostridium paraputrificum
  source 1..912: mol_type = genomic DNA
atgaagaagtacctgaaaatcattagtagtattctgatctttagtctgctgagtccactgagcatcagtgccaaagaggttgaacaagtgtatctgagtctggactatgaaaatacggacagcctgccgcacaactttcgtaaaaccacggatctgagtaatgttattaagggcggtggcaataccacgggtctggaaaatctgaatatcagtggcagtagccaattcacgagtctgagctttctgaacctgaaaaaaaatatcaataccaaaaatgaattctgggacattgatctgcgccaagagagccatggtttcatcaatggtaatgccgtgagctggtatggtccaggcgacaaggcgaacgccggtctgaattataaggaagttatcgccaaagaggccaagcaactgagtgaaatcccattcggtaaaagcattacgctggataacggtaaacacacgctgatcccaaccgtggtggagaatgagagtaagctggtgagtagtaataacgttaaatatctgcgtattgccgtgacggacggtgaccgcccgaccgacgaaagtgttgaccagttcgtgaagtttgttacgcatctgccgaacaattactggctgcacttccactgtaaggagggcatcggtcgcaccacgacgtttatgagtctgtttgatatgatgaaaaacagtaagaatgttagtctggacgatatcatcaagcgccaattcctgctgggcggctttaatctgttcaaaccgggtgacgagaacaagcgtgtgctgttcctgcgcaatttctacaaatatacgaaagagaataacgacaatttcaagaccacgtggagtgattggattaagcaaaataacatcaccccatataccagcacgaccgagctgccgaaaaattaa

SEQ ID NO: 155 - DNA, 915 bp, Clostridium pasteurianum
  source 1..915: mol_type = genomic DNA
atgcgcaagcgtatcaagctgttttggattacgttcttcattgcgatttttagtctgagttttatctttgagaatgtgaacgcggttagttttgccccgaaagaactgcgtctgaccctgaatgcgaataacacggttaccctgccgaaaaattttcgcaaaacgacggacagtgacaagattaaagacatcgataagagtgtgaacctggagggtatgaataaactgaacatcagtggcagcggtcagtttagtgagaagggcctggagatggccaaggaaaatatcggtgagaaagttccaatcacggtggtggatctgcgcgaggaaagtcatggtttcctgaacggtaatgccattagttggaccgacggccacaataaagccaataaaggtctgatcgaagcccaggtgattaaggacgagaacgaacgtctgaaaaagctgagcgaggagaagacggtggagatcaagaatcgcaccctgaacgttgaaaaggtggaaaatgaagaaaatctgacgaaaaagcatggtattagttacacccgcattacggtgacggacaaggaagcgccaagtaaggaggccgttgacgaatttgttaactttgccaagagtgtgccaaatagtggttggctgcactttcactgtaaggccggtaaaggtcgcaccacgacgttcatggcgatgtatgacatgatgaaaaatgcgaagaacgttagcttcgaggatattatcaaacgtcaatttctgctgggcggtgaaaacctgctgaaacgtaccacggtggagaacattaagggcacccgcgcgaagttcctgaaaaatttttacgactattgtcgtacgaacaatgataattttaacacgacctgggagcaatggctgaagaacaacccggacaatagtcgctaa

SEQ ID NO: 156 - DNA, 927 bp, Clostridium perfringens
  source 1..927: mol_type = genomic DNA
atgtttaaatacttcaagaaaaatgcgctgattatttttctggttctgagtattttcacgagttttttcatcaacaatacgagtatcgtgctggccgaggacaataatgtgcacctggtgctggatacgaagaacattaataatctgccaaataactttcgtaccacgagtgacctggagcgcctgaaaaatctgagtaacattaatacgaagggcctggatacgctgaatattagcggtagccagcaattcagtccgaataacctgagcctgctggtgaccagcatcaaaaccaatctgccaattaccgtggttgacctgcgccaagaaagtcatggttttatcaacgagtacccggtgagttggaagggtacgaaaaatgcggccaatctgggcctgacccgtgaggaagttatcgacacggaacgcaacctgctgaatagtattaccctgggtacgccgattcagttcttcaacaatccgaagctgaccgtgatcccagaaaaggtgctgagtgagaaccagctgattaaggcgaatagtatggattatattcgtatcccggttacggacggtaaactgccgacgtatgagatggtggattttttcgttcaatacatcaatagcatgccaaaggacagttggctgcactttcactgtaaggaaggcatcggccgtacgacgacgttcatgattatgtatgatatcatgaaaaattataacaacgccaccctggatgaaatcatttaccgtcaactggtgctgtttggtctgacggaaaagaatttcaatagctttctgagtaaggaacgtctggattttttcacgaagttctaccaatatgttcaagaggaaaatacggacttcaaaaccagctggagccaatggctgaacaagaacaatttcccgctggtgacgattcgttga

SEQ ID NO: 157 - DNA, 927 bp, Clostridium botulinum
  source 1..927: mol_type = genomic DNA
atgtataaaaaagcgaagctgaagctgtacatcccaatgctgctgctgattaccatctttagcagtttcttcctgaatttcaacattgcgctggcctttaacgagccgaacaataagagtgatgttcatatcattgtggacaatctgcgtacgaataaaatcccgagtaatttccgtaccacgagtaatctgacgaacatcaaaaacaatagcagtctgaatctgaaaggcctggaaaccctgaacacgagcggtagtcaacaatttagcaaggacaatctggatattctgaccaaaagtatcgatagcacgctgccaattctggttattgatctgcgccaggagagtcacggcttcgtgaatgaatttccaattagcttcgcgaatgagaagaacgacgcgaatctgggtctgagcaagagtgccgttaccttcacggagaaaaaggatctgaaaagtatcaagctgaataccccgctgacgttctataagcacccagaaatcaatgtggttccgaaagaggtgctgagtgaaaagcaactgacgaagagttatagcctgaattacagtcgcgttccggtgacggacacgaaactgccaaccaacgagatggttgactgcttcattaatatcgtgaaggagtgtagcaaagaaaactggctgcattttcactgcaaagcgggtttcggccgcacgacgacgtttatgattatgtatgatatgatcaagaattacaataatgccacgtatgacgagattatcaagcgtcaattcgccctggccaatttcgacgaaaaagagattatcgaactgagtagtagtgatcgcatcaactttctgaatcaattctacaactattgtaaagacgtgaatggtaattttgatacgacgtggagcagttggctgaatgacagtcagaagcactga

SEQ ID NO: 158 - DNA, 936 bp, unidentified
  source 1..936: mol_type = genomic DNA
atgaagaacatttacattctgctgtttctgttcagtctgggtgttgtggcccaaagcttcctgtatcaacatcaaccaacgctgttcccaatcgttgatgccccactgaacagtgagaatcatctgccgaagaagtggcgtagcaccaaacagattagtcaactggttggtaattacgccaatctgaagggcctgacgcaactgcatctgagtggcagtggtcaatttagccaggaggacctgaaaaacatgagccaggaaattaaaggcaaggccttcgtgctggatctgcgcgaggaaagtcacggtttcatcgatggtaccccgattagctggacggacggcctgaattatggtaacgttggcaaaaccctgcgccaaatcgaactggatgagcaaaagcgcctgaaactgacggcccaaaagggtagtattattgtggacctgagcaaggatctgggtgaaaattttcaaaagttctttgtgcgtgaggtgaaaacggagaaggaactggtggagagttttggttatacgtacatccgtctgccgattacggatcatcaccgcccggttgacagcgtggtggatcagttcatcgagattgttctgagcctgccagcggacagctggatccacctgcattgcaaaggtggtaagggtcgcaccaccacgtttatgaccctgtatgacatcatgcacaacgcgcaaacggtgggtctgaatgatatcctgagtcgtcaaaccctgctgggcggtgccgacctggtgcaggccgaaaaaaacgagacctataagcaaaagccggccaaagaccgcatcgaattcattcgtgcgttctacacgtattgtcgcgaggtgccaaactttgaaatgacgtggagcgactgggtgcatcaaaagcaactggttgcctatcaaacgagtagtagcaattga

SEQ ID NO: 159 - DNA, 945 bp, Acidovorax avenae
  source 1..945: mol_type = genomic DNA
atgccacatgtgagcctggcctgtcgtcatcatgcaggtacacgtccagatagaagtgcaggtcgtacagcattgcaattgcgtcaaccagaagcagaaacgcgcctggagctggtttacgacacggagccgccgcacccagcgggtacgatggccttctttcgccatagcacccaagcgagtgaactgccgccgggcatcgacacgcgcggtctggagagtctgcaactgagcggtagcgaacgtattacgagtgtggagcaggtgcgcgccattcgtcaagcctgtggcgacgccccgctggttgtggttgatctgcgtcaagaaagtcatgcggtggccgatggtcacagtctgacctggcgcggcccgatggactggggtaatgttggtttgggtacagcagcagcaacagcaagagaagcagaacaactggaggaactgcgccgtcagggtaatgccgtggccacgcacgcggaccacgttaaaggtaagagtgatgagccggcgctgcgtcgcctggacacgacgctggcccgtagtgagcaagagattgttgaggcggccggtgcggactatcgccgtatcgccgttaccgatcatctgcgtccaagtcgcggtgaggttgaccagttcattgatctggtgcgtggtctgccggatggtgcgggcctgcacgtgcattgtaatggtggccgcggccgtaccacgaccttcatggtgctgtacgatatgccgcgtaatgcccgcgaagccggtgcggatgcgatcatggcgcgccaaagtcgcctgggcatggactataaccgttgcgcctatcgcccaccagatgcctgtggtttccaggttaagttcagcgaacgtacgctggcggtggttcgcaccagttggcgcagtccaatcaatccgctgattccgcgtgtgagtccgatcgactgtagttaa

SEQ ID NO: 160 - DNA, 945 bp, Clostridium sp.
  source 1..945: mol_type = genomic DNA
atgaaaaaaagcacggcggttttcttcgcgttcattctggtgttcatcagcctgagtagcacgacgctgagtctggataccgccgccgtggccgcgagtgacgaagttcagctggccctggatagcctgaatgagagtacgccacgcaactttcgcaagacgagtaccccgatttatctgacgaaaaatgagaaattcagtctgaaaggtctggataagctgaatatcagtggtagtcaacagttcagcgaaaaaaacctgccaatcctgctggaggaaattggtaccagtaaaccgattacgattgttgacctgcgtcaggagagccatggttttatcaatggcctgccggtgagttggaagaacctgaataatgatgtgaataaaggtctgacgcgcgatgaggtgattgaaaaggaggccaatcaactgagtagtatcccgctgaatagtccggtgacggtgtatcgcgccccaaataatatcaagattccgacggaagtttttaatgaagacgccctggtgaccagtaagaatctgagctatatccgtgtgccggttacggatctgggtctgccaaccgacgatatggtggactattttatcgaagttttcaaaaacctgccgaaagatagttgggtgcactttcactgcaaagccggcgttggtcgcacgacgacgttcatgatcatgtacgacatgctgatcaattataaccaagttagtgcgaccgacattatcgaccgtcagctgaagctggccaacttcgatgaggtgacgatccgcgaattctacaccccggagcgctacaaattcctgctgaacttctataattattgtaaggagaacggtggcagtttcaagatgagttggagcacgtggagtaaggagtacgacggcttcattagtctgaataaagacacgggttatctgaaggtgagttaa

SEQ ID NO: 161 - DNA, 978 bp, Clostridium sp.
  source 1..978: mol_type = genomic DNA
atgaaacagaatgaaattacgctgaataatagtacgaactataataccatcgaggataattttcgccatggtcatcacggctggtatagttatcgccacccattcctgtatgaatattgttaccatatcaacgatgagatgattcaagaaccatactataagagccgcagtaataaactgcacctggtgatcaacagcgacaacgccaaaaaactgccagacaatttccgtaacaccatcgacctggccaacttcaaggataataaggtgatcaatctgaacggtctggcgaatctgaacgccagtgcgagcagtcagttcagtgaaagtggtctggccttcgtgaaacaaagtattggtcacgccatgccaatcattatcgtggacctgcgccaggagagccacggcttcattaatggtgttgcgattagctgggcggacaaccataataaggcgaataaaggtctgacgaaagaagccgtgctgaccgatgagaatacgcgtctgcacggtatcctgctgaataaaccgatttacatcggtgatatcaccctgattccgaagaaactggagaatgagaaaagcctggtgcagagttatgatatgagctacatgcgcattccagtgacggacaacgaaatgccgaataacggcatggtggattattttatcaaattcgtgaatagcctgctgccgaatacctggctgcattttcactgtaaggccggtctgggtcgtaccaccacgttcattgtgatgtatgatatgatgaaaaatgccaagaacgtgagtctgcaagatatcatgaatcgtcaagttctgctgggcggtaaaaatctgctgcgtgacgaacagctgctgatgaatcgcagtgagcaacgcgttaagctgattaaacgcttttaccgttattgtgtggagaataacgataactttaagaccacgtggagtcagtggatccgtcagcatcgcaattga

SEQ ID NO: 162 - DNA, 999 bp, Acidaminococcus sp.
  source 1..999: mol_type = genomic DNA
atgctgcgcgtgggcggtctgtttttcattggtggcaaaatgctgaagaaggtgccgtttctgctgatgctgagtgcgagtctgatcgcctttagtgcgccactgccatgcgtgcgtgcgaaagaagccccgaagagtgtgccactggaggcgccatttagctacgagggtgccctgtggcgcctggatgccgagaacgcgccaggcctgccacgcaattatcgtacgtgtgatgacgcctatcgtgccattccaccgcgctacgcgaaagaagcgagtagtcgtatcccaagctgtaagggtctgagtgagctgcacatcagtggtagcagtcaatatagtgaaaaaggtctggacgccatcctggccgacctgcgcaaccgcacccagggcccgattgttctggtggatctgcgccaagagagccatggttttgtgaatggcatgccagttagctggtatggtaagcgtaattgggccaaccgcgagaagggtcacttcgccgtgctggccgacgaagcccaacgtatccatgcgctgcaaggccaagatgttacgctggtgcgcctgaagacggataaacgtagtgcgagtaccatgaccgttgaggccggccaggttctgacggaagcggagctggccgcccagaaaggtgttatgtacgtgcgctttaccgccacggatcacctgtggccggacgccggtgaaattgaacgcttccgccgtttcgtgaagaccctgccaaaagacgcctggctgcatttccactgtgtggcgggtgaaggtcgcacgacggccttcatgacgatgtatgacatgctgaagaatccggacgtgccgtatgaagacattgttctgcgccaactgcgtatcggtggcgtttacaccccgtttctgggcaaaggtctgagtacctggaagaagccgtattaccgcgccaaaaaacgcggcctggaggccttctatcgtaagctgcataaaacgcaagtgaccgactga

SEQ ID NO: 163 - DNA, 999 bp, Anaerovibrio lipolyticus
  source 1..999: mol_type = genomic DNA
atgctgatgaaaaaaaatcgtatcgagaagagtgcctgtagcatctttgccgtggtttgtagtctgctgattatgctgatggcgaccgcccaagccgccgaaaaacaagaagcgccgatctggcgcctggatagcaagccaggtgccgttctgccacgtagtctgcgcttcatgacggacgagttcccgcaagacctgaatagcgtgccaagtcgcaagggcatggacacgctgcgctgcagtgcgagcgccgaatttagtggttttggcctgagcatgattcgcgataaaatctatgagcatgccggtaaaaatgcggttatctatatggtggacctgcgtaaggaaagtcacggcttcgtgaatggtgaattcgccgttagtcattactttaagaaaaatctgggcaaccgcaatctggccagtgcggccgttccacaagccgaggaaaaagccctgcagagtattatgggtcaagagatcacctttgtgccgctgggcaagaccgacacgaaactgttcacggccagcacggtgaaggtggaacaaattgaaaccgaggaagcgatcacggcccgtctgggtattcactataagcgcattccgattgccgatcaatgtgcgccaagtgacgaagatatcgatgaatttatgaacttctacaaaaatctgccaagcaatagttggctgcactttcactgccacgccggccacggtcgtaccaccacgttcatggtgttttacgatattctgtgtaacccagatgttgccctggacgatattgtggcgcgccaacatgccctgggtggcacgaatctgttcgccccgggtaaaaagaacaattggaagggcgaggaaatccgtaagcgcgcccagcaagtgcgtaatttttacgcgtacgttcaagccaatcatcgcaacaagtatgcccaaccatatagccaatggatgaaagcccaaaatagtcgcgtggacaatgcccagaagagcaaaagttaa

SEQ ID NO: 164 - DNA, 1020 bp, Cellulophaga sp.
  source 1..1020: mol_type = genomic DNA
atgaacaagtacattaactacatcggcgttatggtggtggttctggcctttgcctgcaaagagaacagtctgccagcgattgcgccgaacgtgattacggaaaagaccaaaaatgataccgacgatccggcgatctgggtgaatctgaagaacgccgagaaaagtatcgtgttcggtaccgataaggaaacggacggcggtgtgtacgcgtttgacctgaacggtaaaattatcaagaataaaagtattaccggtgttaaacgcccaaacaacgtggatgttgcctataatttcaaaattaacgacagcacgttcacggatgttctggtgtttacggaacgcgaaaaacaacaaatccgtatgttcagtgttccggacatgatgccgctggacaatggcggctttagcgtgttcaagaacgaaaaaaatctggagtacaccctgccgatgggtgttggcctgtataccagtagcgttgacaacaccctgtatgccatcgtgggtcgcaagaatggtccgaaagacggctatctgcatcagtacaagctgagtacggacacgacgggcgtggttcagagcaccctggtgcgcaagttcggtgtttttagtggtaagaaagaaattgaggccattgccgtggatagtgaactgggctatgtgtactatagcgacgagcagcatggtatccgtaagtactatgcggaaccaacgaaaggcgataaggaaatcagttgtttcggtggtgagctgtttctgagcgacatcgaaggtattgccatcgcgaagcaagcgaagggtaaaggttatattattgttagtgaccaacaaaagggtcaatttaacatcttcagtcgcgacacgaataaattcgtgaaagccgtgaacctgagcaccaccgagacggatggctgtgaggtggttacggttccgctgaataacgtgtttaaaaatggcctgttcgtggccatgaacgacgcgaaggatttctacttttatgatctggccaaactgggtctgtga

SEQ ID NO: 165 - DNA, 1020 bp, Bifidobacterium dentium
  source 1..1020: mol_type = genomic DNA
atgctgatgagtacgcgcatgatgaaacgcattgttggtagtgtggcgtgtgtggcggccgcggccatgctgatgccaggcaccgtttgcccagccctggcgctggagagcgacggtcagtactatagtagcaagcaaccatatgtggcgccgagtgccgccacggtggccagttatagcaaggccccagaaggttacgagccgatctatacggaaagtatggcccgtcatggcagtcgcggtctgagcagttacaaatatgacgcgctgctgatgaaaatggccgaagccgcggagcgcgacggtggtttcaagagtgatgccattaaaaccgagttcatgaagaacctgaatggtatcacggcggccaatgtggaaaatggctatggtatgctgaccggccagggcgcggatcaacattacggcattggtgagcgcgcctatcagcgcaaccaaagcctgtttgaccaagcggccgtggatggtggtacgatcagttatcaaagtagtggtgaggcccgtgccaccgaaagtggtgaaaatttcgagaagggtttcaacgcggccagcggcggtaagctgacggacagtgtggttaaaccaacggatccggcggacagcggcgatggcaaggactttcaaaagaatccagacacgctgtatttccacaaagtgcaaaatccagacggtacggagaaaacgccgggtaccaaagcctatgatattgcgaatgattatcaaaatttcatcagctggccaacgatgcgcagtagtccggtgccgcgtcgcccaagtgccacgatgccgacgagcagtggtccgcgtatgacgtgttgccgccgtagtagtccgaccaatagtctgccgaacagtcgcaccgccaatacgcgcggtaccacgccgccaatggcaccacgtagagttccacgtacagcaagacgtgcaccaacacgtccacgtacaccaatgcgtgcagcaaaagcaccaagacgtagtcgtgccagtacgacgccacaatga

SEQ ID NO: 166 - DNA, 1035 bp, Chryseobacterium sp.
  source 1..1035: mol_type = genomic DNA
atgaagaacattcactacatcctggccctgagtgtgctgccattcgttatcaattgtacgggccaaaaagacctggccgagaagctgaaaccggcggtgatcacggaaaccgttgtgcatgacacggacgacccagccatttggatcaacccccaagacgcgacgaaaagcattatcattggtaccgataaggataccgatggcggtctgtatgccttcgatctgaatggcaaaatcatcaacaaggttagtggcttgaagcgtccgaacaatgttgatctggagtatggttttatgctgaacggtaaaaagacggacatcgcggccgttacggagcgtgaaacgaataaagtgaagctgtacaccctgccggaactgaaagaggttggcgagattagcgtgttcgatggtgaaagtgaacgcggcccgatgggtattagtctgtacaagaacccacaaaccgaagagattttcgccgtggttggtcgcaaaagcggtccagccgacggctatctgtggcaatataagctgattgaaaaagacggtatcattacgggcgaggtggttcgtaaatttggcaagtacagcggtctgaaggagatcgaaagcatcgcggttgacgacgaaatgggctatatttattacagtgatgagcagttcggtgtgcacaagtattacgccgacccggcgaagggtaatgaagaactgagtgtttttggtaaaggtgatttcaaaagcgacgtggaaggcatcagtatttatccgacgagtcaaggtaaaggctatatcctggtgagtaatcaacaaaatgacacgtttaacgtttatctgcgtgaggatcaaagtaagggtaaaatcgccgaaatcccggttagcaccctggagagtgacggtagcgaagttaccaatgtgaatctgggtccaaagttcccgaaaggtgtttttgtggccatgagtaatggccgcgtgtttcatttttatgattggcgtatggtggaaaaggccatcacgagtgccgtgaaagccgtgaagtga

SEQ ID NO: 167 - DNA, 1044 bp, Erythrobacter litoralis
  source 1..1044: mol_type = genomic DNA
atgctgaaaccgtttcacggtttcgcgctgattagtctgggtatcgccgcggcgtgcgccagtattcatgttacgggtgatccagccgtgacggttaccgccgtggcggaaaccgagccggtgggtacggcgaacgaggacgccgccgatgacccggcgatctggcgcaatccggccgatccggcgaagagcctgatcgttggtacggacaaaaaggccggtctgcacgtgtatgacctggccggccgcgagctgagttttatgccggcgccgggcctgaataatgtggacctggttgaactgccggatggccgtgtgctggccctgagcagtgaccgcagcgatctggagaccgcgcatgttagtgtggcccgtctggacccggacaccgccgccctgacgccgattgcgcgtatcgaggtgggtccaggcgaaggttacggtatttgtatgggcgaggttgatggtgacggcagcttcacggtgttcagtgccccgaaaaagggtattgtttaccgcacggccctgcagtttgagggtgaggaactgattgacaagacggagaccctgacgagtgtgccgagtcaaccggaaggttgcatcgccgacccacgtacgggcacgctgtatattggcgaagaggcggccggtatctgggccatcgacatgcagaccggcgacaaacgtctggtggcggaagtggataaccgcctgctggtggccgacgtggaaggtctggcgatcgccccagaaggccgtgatggtggctacctggttgcgagtagtcagggtgataacgcctatgccgttttccgcctgccgggtatggagccagtgggtcgctttcgtattggtgcgggtgcgttcggcagtacggaagagacggacggtatcgagctggatccgcgcgcctttggcccagcctttccaggtggcctgttcgttgcgcaagacggtatgaatggtgccagtgcccagaacttcaaactggtgcgctgggatgccgttctggaggccctggaagccgttccaccggcgccgtaa

SEQ ID NO: 168 - DNA, 1053 bp, unidentified
  source 1..1053: mol_type = genomic DNA
atgcgcctgaccttcattgtttgtgccagcatctgtctgctgggctgcagtacaacacaacgtggtccagcatgggcaggtggtgaaacaccagttgcagttgcagcattggcagaaacgcgcgttgacaccgatccagcggtggacgccgatgatccagcgctgtggagtgacgagagcgatccaacgcgcgccgttatgtttggtaccgataagacggacggtctgtacgttcatgacctggatggtagcgtgcgtcaattcctggcgagtggcgccctgaacaacgtggatctgcgtaccggtttcagtgcggaaggtcgcgatgactttgtgctggtggccgcgaccaacgatgagcgtatgggcattaatctgtatctgttcgatccgcgcacgctggagacccgcgactatggtttcatcgaaacggacattggtgagccatacggtttctgtatgggtcgccgcagtgacggcttctatctgatcgccaacaataagcaaggtgatattaagatttggcgcctggccaacggtgcggagccgggtgccgcggaaatggttcgtcagctgaagctgccgagccaactggagggctgtgttgtggacgatgaccgcgacgttctgtatgtgggcgaagaaaatgccgccatctggcgcttcgattttgatccggcggccagtccaaccccaaccaaagtggcgagcgttgatcgtcagcgcattacggacgatatcgagggtctgacgattatgcgtgacggtcgccaacgctatctgatcgcgagtagccaaggcgatgacacgttcccggtgttccgcattaccgaggaaggtgagacgtacctgggccgctttaccgttgttgcccgcgatgacatcgatggtgtgacgcacacggacggtctggacgcgtggagtggcccaattggccgctatccggaaggtgcgctggccatccatgatgataacgaggagccgctggaaggtcagcaaaactacaagattgtggactggcgcgaagttaaagccgcgctggatctgccataa

SEQ ID NO: 169 - DNA, 1068 bp, Devosia riboflavina
  source 1..1068: mol_type = genomic DNA
atgatcgacctgcgcccggccctggccctgctggccctgagtctgtgctggacgggcacggccctggcccagccggaaccaatcttcgcgagtgtgagcgcgaccctggagaccgagaatgtggacatcgatccggacgacccggccatctggatcaatacggccgatccagcgcagagtcgtgtgatcggcacggacaaagatgttggtctggtggtttttgatctggacggtaagattgtgcagcgcctgagtgacggtcgcctgaataatgttgatatccgccaagacgttaccgtgggcaacgagacgctgagcctggtggccgcgagtcgccgcaatgatgacaccattgttttctacaccgtggacgaaacgggtaccctgacgaaagccacgccgttcgcgtttccaggtgcgccgcgcaaaattgacgacgatatctatggtattggcctgtatcatgacgccgttgcccatcgcctgtttgtgattgccagcttcaaaacgggcgagatcatgcagtgggaagtgacgagtaacgccggtcaactgagcctggcctttacgcgtgagctggcggttccaacccaaccagagggcgttgtggccgatgacgaactgggcttcatttatgtgggtgaggaagatggtggtctgtggcgttttgcggccgcgccggatgccgacgccagtggtcacctggtggacaaagtgggtagtgcgtgtctgccagaggacgacctggaaggcctggccgtggtgaagggtgcggataatagcagttacattctggccagcgcccagggcgtgaatcgttatgtgctgtacgcgcgccaaccggatgccgacggtaatcagccatgttatggcgcgttcaacctggtgacgggtgccaccgacccggtgagtgagacggacggcctggatgttacgaccacggccctgagtgaccgttttccgaaaggtctgatggttgtgcaagatgaccgcaatgaaggttttaaccgtaatttcaagttcgttagttgggccgacgttatgcaggccatgcaactggcggattga

SEQ ID NO: 170 - DNA, 1083 bp, Bradyrhizobium sp.
  source 1..1083: mol_type = genomic DNA
atgcgctgtggcccaggtaagccggttggtctgagttatcagatgccagccccggacagtgacctgggtgcgcgtgccccagccagcagtagcgtggagagtcgcacgccgttccgcatcccgccaaccacgatcccgaaaaaaatcagtgaccaaaacccgccaaccctggtgtatgatatgatcgagccgagcacgtttcgtaccacgagtgacaccagcggcatggagagttgtgatctgaccggttttgacggtctgagcctgagtggcagtgaacaattcagtgaacgtcaactggaggaactgattaccctgacgcgcccgaaacgtatcgtgatcattgacacccgtcaggaatttcatggtttcgcgaacggcgagccgattagctggtatgccgccgacaattataattggggtaacgttgaaaaaccagccgagatcattccagcgaaggaaacgaaaaagctggccaaactgccgaccaaattcccgtttggtctgacgatcgaaagtggtagcgtggtgaaaggtaaggccagtagtgccccgccaaaggttctggatttcggtaaagtgaagattgaaaccgaggaagagctggtgaaacgcaagggcgtggcgtatctgcgtatcccgaccccggaccatgcccgcccaacggatgccgaaatcgataagctggttagtgaactgcgcaatctggcccgcgcgggtgacgtgcacctgcattttcactgtcgtggcggtatcggccgcacgaccctgctgatgaccctggccgatatgctgcataatagtgacaaggtgcaatgcgcccacatcattgaacgtcagatcaaactgcgccgtcgcgacgagttcccaacgagcaaagcgctgaacaaggttctgcgtaatgagaagagtgagctgatgaatacctttttccaatacgccaaagaacagccgctggagaaagccgatgcgatcagctggaccgagtggctggccctgaaggcgcatcgcgagcatcaaattctggacacgagtagtgttggtacaccaccaacagcaccaccaggtcatcgtatgccataa

SEQ ID NO: 171 - DNA, 1089 bp, Algoriphagus marincola
  source 1..1089: mol_type = genomic DNA
atgcacaaaaagctgaattactttctgagtattttcacgctgaccggtctggccagttgtcaagagagtagcctgcagaagggcgacctgccagacgaaaaagttgagctgatcagcccactgcatattagtgacagtgtgatccacgatacggacgacccggcgatttggatcaataaaagtaatccaagtcaaagtctgatcatcggtacggataaagatagcattggcgccctgtatgttttcgacctgacgggtaaaaccatcgatagtctggtgcgtcgtgacattcaacgtccgaacaatgttgacatcggttacggtctgattcacggcaaggacaccattgatttcgcggtgacgggtgagcgcctgacgagcaaaattcgttttttcagtctgccagatatgaaggagctgaacccaggtggcctggaagtgtatcaaggtgaaacgggcgaggactatcgcgacctgatgggtatcgccctgtatcagagtccgaaaagtggcaaaacctatgtgatcgcgggtcgtaaaaatggtccgacggatggcacctacctgtggcaatatgaggttaaaagccaagatggtaatctgagtctggaactggtgcgtaagtttggcaattttagtggcaataaagagattgaggccatcgcggtggataacgagctgggttatatttattatagcgacgaaggcaccggtgttcgcaagtactacgccgagccagacatgggcaacgaagagctggccctgttcgccaccgagggttttacggatgaccatgagggtattagtatttataaaacgagtgaaaccgaaggttatctgctggttagcgaccaaggtgccaatctgttccacgtttttccgcgcgagggcagccaaagtaatccacatcaacacagtctgatcaccacgctgccgctgagcacgctgagtagcgacggtaatgaagttaccagcgccgatctgggcggtgagttcagtgaaggcctgttcgtggccatgagcgacgacaaaacgttccagatctacagctggaaacagctgaaaaaacatatcaatcagaatcaatga

SEQ ID NO: 172 - DNA, 1110 bp, Bryobacter aggregatus
  source 1..1110: mol_type = genomic DNA
atgaaaaagaaccaaacctttctgagtttcttcctgctggcggcctatggtatcgcgcaaaaagagagtccacaaccgaagctgcaatatgccgttatcctgacgcgccacggcgtgcgtgccccactggccccgaatcgttggctgcacttctatagcgtgggtagttggccgagctggggcgttctgccaggccatctgacgccgcacggtcgtcaaatgatgaaactgatgggtgcgtactatggcgaaaccttccgcgccgaaggtctgctggcccgtaacggttgtgaagacagtggtcgcatctacgtgtggagtgatagtgaccagcgcacgacggaaagtgcgcgtgccctgctggagggcattatcccaggttgccgctatgcggttcacagtggtcgtccgggcatcccagatccgctgctgagtacgcaaggtacggcccgctttcgtagcgaggatcgtagtggcctgcagccggcgctggacaaactgcatttcgtgctgaagggtgcccgcgcgcgtagtaatctgagtctgcgcacggccgccacgctggcggaaaacctgatgctggagtacgccaatggctttgaaggtaatgagctgggctggggtcgcctgagccaagaagacctgcaagagatcctgacgctgcgcgccgcccaaccgcgcaaagcggaatatgcgcgtaatctgtggctgcgcatccaaaccagtctgtgccaagccgccacgggtgagagtattgacggtagtattggtccaaagaataccgccctgctgatcctgagcggtcacgacaaaaacctggaaagcctggccgcgctgctgggtctgaattggcaactggccagctacccgaaaaactatgccagtccgggcggtgcgctggtgttcacgctgtggcgtcaagacggtcgcttccgtctgcgtagtcagtacatcgcccaaagcctggagcaactgcacaatggcagtggtggcaccccagtggtgattgacctgggtagcagtgacttcaccccaatcacgcgcccaccgcatggtcatttctgtgttgccacgagtctgctggttacgagtagtaaacgtcgtcgttga

SEQ ID NO: 173 - DNA, 1116 bp, Asticcacaulis sp.
  source 1..1116: mol_type = genomic DNA
atggacctgaagcgtttttatgtgggtttcacgaccgcgagtgccgtggtgatcaccgccaccctgctgacgggctgtgcgagtctgcacagtgagctggttagtgatagtaaactgatcgccatcggtaccggtacgccagtgctggccagtgcggaaacggttgcggtgggcaccgccggcctggacgcggccgatgatccggagatctgggtggacccaatggataagagtcgcagtctgattctgggcacggacaaaaaggccggcctgtatgcctataatctggatggcagcgtgcgtgattttgtggcgcacggtccgctgaacaatgttgacctgcgtacgatctatggtgaaaccggcagtttcaccctgattgccgccagcgaccgtgcgcgcaatggtgccgccctgtttacgctgacggacgatctgaaactgaaagtggcgggcttcctgccaatgaccacgagcgaagcctacggtctgtgcatgggcaccattgaggcgggtattacgatcgtgatcattggtaaaaacggtgatgttgtgcaagccatctatagtgaaacggaggccggtccgcgtggtgaaattgttcgtcgctttaatgtgggtacccagagtgagggttgtgttatcgacgatcgcacgggcgcgctgtacatcgccgaagaggcgaaaggtatttggcgctatggtgtggaaccggcgacgggcagcacccgtgtggagctgcaagccgcgccgagtaatatcctggttccagacgtggagggtctggcgctgctggtggatgaagacagtggtctgagttatctgattgcgagcagccagggcgatagtgccttcgccgtttggcaagtggccggtgagaccagtatctacaagggtcgtttcagcgttttcccaggcaatggtttcgacgcggtgacgggtacggatggtgttgccgccctgggtggtcaagtgggcccgtatgccgaaggtgtggttgtgatgcaggacgacagtgatatggagggcgaaacgccaaccggtgcgcgcgcccgccaaaacttcaaaattgttccgtggatggatgtgaaaaaggcgctgaatatcggcagttaa

SEQ ID NO: 174 - DNA, 1128 bp, unidentified
  source 1..1128: mol_type = genomic DNA
atgggtagcaattacacgatttataagcgtttcttcaatagtcgtaatgttaagcgtctgctgattgtgctgctgtttatcggtattgtgcatgtgagcttcacgtggtatctgtttcatagtgagagtaccaatacgatcaccccaacgatgaaactgctggccgaaggtagcatcgaccaagacgatatgtgtatctggattcacccgacggagagcagtaagagcgtgatcattaccgcggacaaaaaggccgaaaaagtgttcgtgtacgatctggagggcagtgttctgcaagtgctgagcatgccaaagccaggcaatatcgacgtgcgctatggtttcccgctgggtggtaaaaagattgacatcgtggcctacctgcaacgcaaaaagggccatcgtatcattgtttttgcggtggagccgaaaacgcaccagctgaagcgcgtggatgacaatagtatcctggtgggtaaaggtgttggtggtacgctgtatcatagccaaaaaaccgacaagttttacttcatcaagaccgccgagaagaagggtaagggcggtatcgaacagtttgagctgttcgacgatggcaaaagtaagattaccggtcgtaaggtgcgccaatgggatctggccggttgcgaaggtgccgtggccgacgacgagagttgtgcgctgtatatcagtcaagaagataaaggtgtttggaaagtgggtgccgaaccaggcgagccaacgccgggtgaactgatcattaaaacctggaagaatggcctgatgtttgacgtggaaggcctgaccatctataagagccacacgcgcgagggttatctgatggtgagtaatcaaagtcgcaatagcgtgaaggtgtatcaactgggcaaagaggacaagttcctgggtacctttcatattgacggtgcgatccacacggacggcattgatatgaccaatgtgtgcctgggtattcgcttcccaaagggtctgttcgtttgtcatacggaccaagaaggcagtaatcgttgtccggttctggttaccccgtgggagagtatcgcccgtggtctgaacttcaaggtggatgtgagttggaatccacgcagtgagacgtttaaacatttctga

SEQ ID NO: 175 - DNA, 1128 bp, Bacillus licheniformis
  source 1..1128: mol_type = genomic DNA
atgaatttttataaaacgctggccctgagcacgctggccgccagtctgctgagtccaagctggagcattctgccgcgcgcggaagccagtgcctacaaggatttcagtgttaccgccgatgccgagaccgaaccagtggacacgccggatgacgccgccgatgacccggcgatttgggtgcatccgaaacagccagagaaaagtcgtctgatcacgacgaacaagaaaagcggtctgattgtgtatgacctgaatggcaagcagctggccgcctatccgttcggtaagctgaacaatgttgacctgcgttacaattttccgctggatggtaaaaagattgacatcgccggtgccagtaaccgtagcgacggtaaaaataccgtggagatttatgcctttgatggtgaaaaaaacaagctgaagaatatcgtgaacccacaaaagccgattcagaccgacattgaagaagtttacggcttcagcctgtatcatagtcaaaaaacgggtaaattttatgccatggtgacgggtaagaatggtgagttcgagcaatatgaactgtttgacaatggcaagggtcaagttgaaggcaaaaaggtgcgtagcttcaagatgagtagccaaaccgagggtctggcggccgacgacgaatacggcaaaatgtacatcgccgaggaggatgcggccatttggagttttagcgccgaaccgaatggtggtgacaaaggtaagattgttgatcgcgccggtggcccacatctgacggcggacattgaaggtctgaccatctactatggcgaggacggtgagggttacctgatcgcgagcagtcagggtgacaaccgttacgccatttatgatcgccgcggtaaaaatgactatgtggcggattttagtatcgacgacggcaaagaaattgatggtaccagcgacacggatggcattgacgtgattggcttcggtctgggtaagaaatatccgtatggtatctttgtggcccaggatgcgaagatccgcaagatggacagccaaccaatccgtacgagtaagctgagcccgggcaagaaactgctgacccgttggacgacgagtctgattagcatgatccgcagtattccagagaactga

SEQ ID NO: 176 - DNA, 1146 bp, Deinococcus proteolyticus
  source 1..1146: mol_type = genomic DNA
atgagccgtttcattcgcccgctgctgctggttagtctgtgtgccggcgtgtgtgcctgtgccccggttgccacgggtccagcgagtccgcaagcccagaccggtgccggcgcgctgccggtggttgaagcccgtgcccaaacggcgccggtgggtgacccggccgatagcgacgatccagcgatctgggtggacgccgccgagccagcgcgcagtttcgttatcgccacccgcaaggaaggtggtctgacggtgtttgatctgaaaggtcaaacgattcaggacctgaacccgggcggcgttcgttacaataacgtggatctggtgcgcggtttccgtctgggtggcgaaacggtggacctggcggttaccagtgaccgcaagggcgaccgcgtggcggccttcgttatcgacccacagacgcgcaccctgcgcgaggtgaccagcccggccaccccactgctgttcagtccgggtccaggtacggacggtaagcgtacggcctacggcctggccgcctatcgtaccgccgcgggtgaggatcgcgtgctggtgagtcaaaatggcttcccggtggtgggtgaattcgaactgtacgacgatggtcaaggtgtgagtgttcgtccggttcgccgtctggagctgccagccgcgctgccgggtctgaccgtggacgatccacaatttgagggcatggtggttgacgccgaacagggtgtggcgttcctgggtcaagaacaaattggcgtttggcgctgggatctggatggtcgtcgcagtgtgttgttggatcaagttgcaccattggcaccacgtttgcatgcagatgtggaaggtctgacgctggtgcgtggcagtggtggccgcggttatctgctggtgagcagtcagggcagtaatgcctatgccgtttacagccgcgatggtaaacgctatttcggtagctttcaggtgacggccggtagtgatctggtgcaagacagtgatggtgccgacgcggttctgaccccactgggtagcgattatccgggcggtctgctggtggtgcaagatggtgaggccggtggcgccgaaggtcagacgaactttaaactggttagttgggcggacgtggagcgtgccctgaacctgccagatgtgcgttga

SEQ ID NO: 177 - DNA, 1164 bp, unidentified
  source 1..1164: mol_type = genomic DNA
atgaacaacaatcaataccaactggataaagttgtgatcctgagtcgtcacggtattcgtaccccactggagaacaccattgccttcctggagaagagcagtccgttcaaatggccgagctgggaccacgcgtatggttatctgacgacccgcggtggcgttctggaaacgtttttcggccattatctgagtcaatggctggaacagaagaatatcaacattgaaccggagaatccggatatctatgtttatgccaatagcctgcaacgcaccgttgccaccgcccagttcctggtggccggtgcgtttgccggttacgacattccaatccaccataagtataccattgaaaaaatggatccgatcttcgacccaagtgtgaagaatgatagcctggagatcaaacaaaaggtgctgaaagagattgaagaggccgaccaaaagcaaagtattttcaaaaacctggccccggcgtacaagatcgtgagcgacattctggattatccacacagtcaactgtatgccgaactgaaatgcgacttcgcggacatcccgtatgagctgcattttgttaagaatgaagaaccagagctgcgtggtccgctggccatcggtatttgtgtggtggatgcgttcctgctgcagtattacagcgccttcccgaaggagcaaatcgcctggggtcgcatcacgagtcaggaacaatggcaacaactgatgcacattcgcaaccagtacatcgacctggttttccacagccgcaccatcgcccgccacaatagtaaactgctgatcaataaaattgacgacctgctgcataacaaaacgcataaagttaatctgctggtgggtcatgatagcaccatcgccgcgctgctgggcgccctggactttgcggaatatcaactgccgaatcaattcgaaaataccccgattggtggtatggttattctgcaacgctatcgtaataaagccagtgaaaagtacctgttcaaagccgagtatgtgtaccaaagtttcgagcaactgtatacggcgcaaccactggacatcaatcatccaccgcagcattaccagctgcaactgaagaatgccgtggcgaacagtgacggcttttatcagtggcaggacatcgagacgcgtctgaaggaattcaaaaaatga

SEQ ID NO: 178 - DNA, 1167 bp, Bacillus sp.
  source 1..1167: mol_type = genomic DNA
atgctgaaggaaattagcacggccgcgctgatgacgagtgttctgttcagtacggtgaacagcgccgcggatctggtgggtgagtttccgagtctgagtgttcaagagaaacaacgccatagtaagtttgtgaaagttgaagccaaagaggaaaccgcggcggtggccaatgcgggtgacgccgcggatgacccagccatttgggtgcacccgcatgacagtgaaaagagtaaaatcatcggtaccaacaaggaagaaggtattagtgtgtatgacctgaaaggtaagctgctgtacagctataattttggcaagctgaacaacgttgacgttcgttatggtttcccaattggcggtaagaaaattgacatcgcggccgcgagtaatcgtagcacgaacaccattgatatcttcgcgatcaatagtaagacgggcgagctggaaaatattacgggtagcccgatcaaaagtaatatgaaagaggtgtatggttttagtctgtatcacagtcaaaaaacgggcgtgttttacgccctggttgtgggtaaagacggtacgttcgaacaatacgagctgttcgacaacggtaagggcaagattgagggtaagaaagttcgcgagctgaaactgagcagccaaagcgaaggtatcgtggcggacgacgagtatggcacgatgtatattggtgaggaggatgttgccatctggaagttcaatgccgaaccagatggtggtaagcaaccgatcaccaaggtggacagtgcggacggcaatcatctgacggccgacattgagggcctgacgatctactacggcaaaaatggtacgggttacctgatcgcgagtagccaaggcaacaatacctatgccgtgtacgaccgtaaaggtaataatgaatatatcggtaactttgcgatcgttgacggtaaaaacaccgacggcaccagtgataccgacggcattgacgtgatgagcttcggtctgggtaagaaatatccaaatggtattttcctggcgcaggatggcgagaacatcgagcgcggcaagatcgtgaatcagaactttaagatggtggactggaaaaagattgccaaaggttttgatagtaagctgcaaattgaaaataatgtggatccacgtaaactgaaattccgccagaatagttga

SEQ ID NO: 179 - DNA, 1176 bp, Saccharicrinis fermentans
  source 1..1176: mol_type = genomic DNA
atgcagtttattaatctgaccacgctgtttatcgcggcggccagtatcctgagtagctgtcagccaaagggtaaccagaataataatagtgaagcggaacgcgataatacggccattctggagaaaaacaaagaggcccgcgatgatagcctgaaactgaccgaggcgtatgccctgcaggcgcagttcagtaacattgttagcgccgactatgaaacgacgccagtgaagagtaatctgggtgatgacgcggccgatgatccggcgatctggattaacaaaatccatccagaaaaaagtatgatcattggcacgaataaaaaagccggcctgaacgtttatgacctgcaaggcaatgagctgcagtttatcccgatcggtaagatgaataatgccgatgtgagctataacttcgcctacaaaggccaacaggtggatctgctggcgggtagtaaccgtaccagtcaaagcattgacgttctgctgatcgatggcgccaatcaaaagattatcgagaaaccactgtgtagtatcccaagtagtgtggatgacgtgtatggtctgtgcatgtacttcgatgccgtggccaataagcactatgtgttcgtgaatggcaaaaatggtaagattgaacagtggctgctgaaaaatgacaacgatagtatcaagggtgagctggcccgcagcttttgggttagtagccaaccagagggtatggtggttgaccatgtgacgaatacgctgttcgttggcgttgaggaagatgcgatttataagttcaaagcccaagcccaggccgacacgagcagtattcgtctgaccgccagttgtaacgcgaataacgcccacattagctacgatattgaaggtctgacgatctatcgcatcagtgatacgaaaggttatctgctggcgagcattcaaggtaattttagttatgcgattttcgacctgagtgaaaagaataattatatcaccagttttattattaaagacggtgtgtttgacggtgtggaggagaccgacggcattgacgccaatgccagcctgatgggcccgaagttcccaaaaggtatgctggtggttcaagacggttttaacaaggacaaaaaggagaatcagaatcagaacttcaagatcattagttttgaacgcatcctgcagtttctgcagtaa

SEQ ID NO: 180 - DNA, 1212 bp, Asticcacaulis benevestitus
  source 1..1212: mol_type = genomic DNA
atgacgccgagtcgcattctgaccttcgcgaccctgtttgcgtgcctggccgttgtgacgccatgtagtacggcgagtgccaaagaaaatacgttcacgctggaacgtgtggttattctgatgcgccacggtattaaacgtccgaataatgacccgccgctgccgaagcgcttcacggatcaggcgtggccagtttggagcgtgccgccagccggtctgacgccgcatggcgaacaagcgattgcccgcatcgccgacttcgatcgcctgacgtacgcgggcctgctgggtagtgactgtccgccagccggcgcggttcgcgtgatcgccgacaccgaccaacgcacgatccgcacggccgaagtgtatgcgagcacggttttcaagggctgtgacgtgcgtgttgaacatgcgggtgaaggccatgccgacgcccattttagtccgttcaacgaggatgtggccagtccgccactggaccgcaaagcctttatggatgaggccctgacgagtggtggtatggccgccatcgacaaagcccaccatgtggattatgccctgctgagtgaagtgctgaatctgaaggacctgccgggttgtcagatcgataaggtgtgcagtctgagcgacatgccaagtgttctggatgttagtggccgcgatccgaaggtgagtggtgcgctgaaaacggccagcagtctgagtcaaatcctgatgctggagtacgccaacggtttcccgatggatgaggttggctggggcaaagtgagcgagagccagattaccgccctgagtgccctgcacgcggaggaattccgtctgatcgcgcgtccaaaagccgttgccacgtacgcgagcggtggtctgctgaaggacattgaggcggccctgttcggtccggacgcgagtccatataccctgctggtgggtcatgacggcaatatcgcctatgttggtggtgcgctgggcctgcactggcaagcccaaggtttcgcggccgacgacccgccaccgggtggtgccctgatttttgaactgtggcgtgatggtgccggcgaggcctttgttcgcgtgcgttttcgtagcagtagcctggagggtatgcgtaacctgacgcacctgaaacgcgccgccagtacgcgcattccaatgccgctgtgtgacgtgaaggccgaatgtagcgccgcgaccttcaaagccctgatcccgtga

SEQ ID NO: 181 - DNA, 1218 bp, Amycolatopsis orientalis
  source 1..1218: mol_type = genomic DNA
atgcgtatcgccgcgctgctgccactgctgctggccgccagcctgctgacgggcgttccggcggaagccagtcaaaatccgagcccagtggcccaaacccgtgccttcgttgacgacgccggcgcggacccagcgaatgccgatgccgatgacccggcgatttgggtgcatccagagaatccgagtgccagtgtggtgctgggtacgctgaaagagggtggcctggcggcgtttgatctgaaggcccgccagctgcagcatctggcggtgccagccggtggccgttttaataacgtggacgttgtgggtgacctggcggttgtgagtgaccgtggtcgcgaccgcgtgcgtgtgtatcgcattgatccggccggtgccgccgccggtagtcgcgtgctgcgtgatgttacggatccagccgcggccccggtgttcagcgccagtgagagtgaggtggacgagcaacgcacggcctatggcctggccgccggtcgtgatccgcgcaccggtacgcgttgggtggccgttacgcgtcgtcatgagacccgtgtggccctgctgcgcctggtggacaaaccggacggcacggttggtaccgccccgatcggtaccatcgatctgccggcgagcttccgcctgccaaacggccgtacgtggagtccatgcggtgaaccagatgagcgcccgcagctggagggtagtgttctggacgtgggtcaccgtgtgctgtatacggcccaagaagatgtgggtatctggcgtattccgctgggcgaggagggttttggtcgcccagcgctgattgataaagtgcgtagtttcggtgtgccgcaacgcttcgacgaagccacggaggagtgcgttgcggacggtccagacccgggttttggcggtaaatggctgacggcggatgccgagggtctgacgctggcggacggcaagctgctggccagtagtcaaggcgatagtcgcttcgtggtgtacgatcgcgcgggtaccccgcgtcgtgacttccgcatcgtggccggtcgtggtaccgatagcgttgagcatagtgacggcgcggccatcacgacccgcagcctgggtccgctgtttccacatggtctgctggtggttcacgacggcgaacgtcgcccggccgccacgggtccaagtggtgaggagctggccacgaccggtttcgcgttcgtgcgcctggaggcggtggtgcgttaa

SEQ ID NO: 182 - DNA, 1227 bp, Asticcacaulis benevestitus
  source 1..1227: mol_type = genomic DNA
atgacgctgaaacattggctgcgcttcgccctgacgttcagcggtgcgtgcctgctggccatccaggcgagtgcccaaccaaccaaagttgaacgtgtggttatgctgatgcgccacggcgtgcgcagtccgattacgggtgaagcgccgctggatacccaaaccggtgccccgtggccggtttggagtgtgccaccggagacgattacgccgcacggcgcggaggccctgaaggccctgggcaatgtggatcgcgcgtggctgagcgcccgtggtatcctgccagccaaagcgtgtccggacctggccaagaccattgtgtggacgaataccagtccacgtacgatcgccacgggtcaggcgtatgttcaaggtctggcgccgggttgtgccctgcatgtgggccacctgccagaagaccaaattgacccgctgttcgagccgacccgcgcgccaccgccatggttcgacgcccagcgtgccgttacgagtatcaatgcctacacgggcggtatgccagttctggtgcagcgccatgacgccacgctgggccaactggaacacgtgctggactgtggtgccagtccctgtagtccacaacgtgccccgcgcctggcggtgacggccgataatcgtggcctggtgtttgagggtccagtgcgtgacgcgagtggtaccgccgaagttctgatgctggagtacctggaaggcttcccactgaaggaggttggttggggtcgcgccaacccggcgacgctgaagaccgtgggtgaagttcatgccgcgctgtttgacgtgttcagccgtccgccgtatatgatggcctttcagacggaaccgaccgcccgtcgcattatcgacgattttagtcgcccagacgccccagatttcgacatgctggttggtcacgatacgaatgtggcggccctggccgcgctgctgggcgttaccgtggaagcgccgggttttgccgttaatgacccgagcccgggtggtgccctggtgctggcgctgattcgtgatgcccaaggtcgtgcgttcgtgcgcgtgtattaccgtagtcaaagcgcggacgatatccgtgccgcgcgcgatcatgcgacgtggaagagtctgacgatgaacgcctgtaagcaaggcccacaacacatgtgtccgctgccggatttcgttgccctgctgaaagcaggtacagcagaagcacgtgcaccattggttgcaagataa

SEQ ID NO: 183 - DNA, 1272 bp, Acidobacteriaceae sp.
  source 1..1272: mol_type = genomic DNA
atgctgttccgctttgtgctgccggcggttctgctgctgccggcgttctgcagctttgcgcagacgccgaccgatgatgtgcgcgccgttgtgattgtggcgcgccatggcgttcgtgccccaattgaaaatgaaacgcgcagtagcgtgttcaacgcccaacgttggccggcctggccgagtgccccaggtgacctgaccccgcatggcatcgccgccctgaaacgcatgggtgagttctatcgtcaacgctacgccaccctgctgcatgactgcaatagcgttgccgtggaaagtacgaataccagtcgcacgatcggcagtgccaaagcggtgatgagcgttgttgtgccagactgtgacgttcgcgttatggccgatctgaccgccgacccgccgagtatcgataaggccaagctggccgacgccacgcacggtcgcatggcggatcaaccagagtggttcacccatgcgttcgcccgcccgctggaagaaatgcacgacgtgctggcgaagtgcgccggttgtaaacaggttccagactttcgcacgagtatgctggacccaagtgcccacagtgaaaatatgatgcaagcccaaggcgagaaggccggtgcgctggtgccgcgtgacccacgcaaagaaaatgccgccgccctgggcgccgattttagcgagaacttcctgctgcaatatgcggaaggtatgccgatggaccaagtgggttggggtcgtgtggatcgtgcccgcctggacgatctgatggaaatgaatacccgttatcacgactttattctgcgcacgccgtactacgcgcagcaagccgccggtgcgatggcccagaagattagtgactggctgagtatggccgccattctgaatatcccagtgcgtccaggccgcccggatgttccggccggtggtcgtgagagtgtgctgtatctgagcgtgcacgacgcgaacctgacctggctgggtggtctgctgcgcatcgcctggctggttagcgacgaaacgctgaatgccaccccgccgggcagtgcgctggtgttcgagctgcatcataatcgccaaagtaatgccgatacggttcaagtgttctttattgcccagacgctggaccagatccgtaacctgaccgatctgacccgtgcggagaagccaagtatcagcccaatctatgtgccaggttgtagtggcccgggtccgggctacgcctgtagtatggaagccttcgttcgtgtgaccaaggaggcgacgaaggtgccgtga

SEQ ID NO: 184 - DNA, 1281 bp, Bacteroides sp.
  source 1..1281: mol_type = genomic DNA
atggccgaaaacccactggatgagattcgtcgcgacccggaacgcgcgggcggtgtttactacgtgacggatctgagccatccggtgaccccagcgccaaaaggttatacgccattctacatcaatggttatttccgtcacggcgcgcgtcagatcgacgatgaggtgacctatccagccatctatggcgtgctggagaaagcccatgcgacgaacaatctgaccgactttggtaaagccctgtacgaacgtctggaaccgtttaaaaaaaatgtgttctacaaggagggtgatctgacgcagattggttatcgccagacccgtgaaatcggccgccgtatggtgcagaattatccagaggtgtttgaaggccatccatatctgaaaacgaatgcgacgaatgttctgcgtgtggccgccacgatgcaaagtgttaacagtggtatcctgagtctgcgtccaggtctggaatgggcggaaatcgacaatagccgcagtttcctgacgaccctgaatccgtatggcaatgtgtgtccgggtcgtagtccgctggacaagtatatcctgggtaaggagaactgctggtacaagaaataccgtagctatatcgacgaaaaactggatgttgatgcgtttttccgccgtctgttcattgatgtgacgcaggttgagagcgagtatgacaagtacgatctgatccaccgcttttggctgatggccagtctgatgcaatgtctggaccgccaggttccaatttgggatatcttcacggaagaagagatcctggtgtgggcggagatcgagaattataaatacttcgcccaaaagggtccggagccagttagtcatggccgtagctggggcctggcgagtcgcacgctgcgtcacctgctggacgaaagtgcggaggacctggttcgcaaacgccatggtattaacctgaattttggtcacgacggtgtgctgatggccatcctgacgaacctgcaggcgggtacctgggcccgcgaagcgagcaatagtaaggaagccctgcaaagctggaaatactgggacatcccaatgggcgccaatctgcaaatgatcttctatcaaagtgagggtaatccagatgttctggtgaagttcatgctgaatgagaaagatctgcgtctgccgctggaggccgttgaggcgagctattacaaatggaatgaggtgtacaagttctacatcgagcactgcgacaaagtggagaaaagtctggccgaaacgctgaaactgagttatgaggatttttga

SEQ ID NO: 185 - DNA, 1299 bp, Barnesiella intestinihominis
  source 1..1299: mol_type = genomic DNA
atgaagaagcgtacgctgaccgccggtacgattctgctgtgcctgagtgcgatcagctttggtcagaacagtattaaagacttcctgcaccaaaatccacagtttttcggtagtacggccagtgtgtactattgtaatgatacgacggacaccccggccccaaaggattttatcccgtttcacatcgaccacattgcccgccacggtagtcgtacgcacgatagtaaaagtatggtgccaaacctgtataaactgatgaacaaagccgacagcctgaatctgctgacgcgcgaaggcaagctgctgcgtaaccaaattgacaccatctaccatctgatgaatcaccgctggggtgacctgacgccgctgggcgcccgccaacaccgcgatatggcccgtcgcatgtaccatcgtttccgtccagcctttaccccgcaggacggtaaagttacgctggtggcgcaaagtacgacggttccacgcagcatggccagtatggcggcgtttgttgccgagatgcgtggctataccccgacggcggaattcagcatggacccaagtaacggctatgataataccctgcgctttttcaaaggtaaagagtaccaacaatatctgagcaagggcagctggaaaaagatcctgcgtgcctatcaagagaaacacacgccaacccgcctgatcgaccgtatcttcaaaaaaggttgggaacaaatcatcccagatccgatcacctttatgacgcacctgtatgcgctgacgattatcctgccgaatacggactatgacattagtctgtatccgtggtttacggaggaggaaaagtttgatctgtggagcgccaacaatctgagtcagtatctgcgcaaagcgaatagcatcccgggtaagggtctgccagtggccattgcgaagccactgctgaaagatatgctggccaccagtcaagcggccattgacggtaacggtgttgaggccaatctgcgcttcgcgcacggcgaaaatacgatcccgctgctggccctgctgggtattgagaacgccgccgttgtggaagccgacccggagaaggttacggaagtgtggcaagactttaaatacaatccgatggccacgaatatccaatggattctgtataagaacacggacggcaagatcctggtgaaagttctgtttaatgagcaggaaaccaagctgccaattgatagtgagtatgccccgtattacgattggaagctgttccaaaaatattgcgaaaaacagatggccaagtatccagacacgaccccagccctgtga

SEQ ID NO: 186 - DNA, 1302 bp, Endozoicomonas elysicola
  source 1..1302: mol_type = genomic DNA
atgaaactgcaaacgctgctgggtcattgtctgaagaaggttatggccttcgactactgtaataacagcctgaaaagttatctgatgcacggccgtaccagtggttgctttcatatttgtagtctgccagaaagtggttttaaaaagctgttcctgatctttcacaccgtgttcgccatgtgcttcctgagtgtgggcgttgacgcgaccggtattggtttcgagatggaaacgccggccgattatctgatcagtctggtgcgccatggcgaccgcagtccgcgtgacctgggtgatatggcgcagtactggccgatgggtccaggccaactgaccgccggtggtctggagcaggagtatctgctgggtaaaaagatccgtcaacattactttagcgagagtctgccagacagttggagtccgaaaattagtcaacactatgccaagggtctggatcgcacgatccaaagtgccagtgccctgctgcaaggtgtttacccaggtcagccgcgtaacacgggcctgccgggtggcatccaagtgccaccggtttatgcgagtccgctggccagtgacgacctgttcagtgcccaacgcctgtgtccgggttacctgcatcgtgtgcaagcgctggagaagagtgccgactggctgcgcaaaaaagaacagtatcgcgatcaactgagcagttggtttgagctgagtgagggtagtggcgcgggtgatctgtatagcctgattccgctgatcgaccagatcagtattcatcgtatgcatcgccgtccgatgccaaaaggtatcacgattcaagaagccatcgaactggaggaactgctgaattgggttgtgagtcgtatcattgccaattatgaaattgcccagctgattggcgccccactggcgaaagccatgattcgcgatttcaagcgtgttcagcagtgtctggaggataaaggcagctgcaatagttgtcaacgttggacgctgtatagtgccagtgacagtaatctgctggccatcatgacgatgctgggtgccccgagtgaccgtattgtggattatgccacccactttggtgtgcaactgaattggaatgaaggccgcccagagattgcgctgagtctgaaccatgagccgttcagtgtgccgggttgtgttggtcactgtagtctggaccagtggctggtgctgctggaacaaagtctgccggacgactgggattatctgtgcgcccgcgaccgtggcggttttgccccatatccggagccgtatgttccaagcggcagtgtggccagccgctaa

SEQ ID NO: 187 - DNA, 1323 bp, Asaia astilbes
  source 1..1323: mol_type = genomic DNA
atgctgatgattggtagcccagtgaatgccctggccgccctgtggggctttgcgctgagtagtttgttgttgtgtggtgttgcaacaccagcacgtgcagttccattgagtgcagcaccaccaagtgcagatgcagttctggagcgcgttgtgctggtggcccgccacggcatccgtagtccgacgcacgacccagcggccctggcgcatgaaaccggtatcgcctggccggaatggccggttagtccaggtcaactgacggagcatggtcgcgccacgctgagtgttatgatgcgcgatattggtcgtcactatgatctgggttgtagtacccacaaagagagttgtctgacgcaaacgcgcccggtgatctgggccgacagtgccgacgaccgcacgcgcgaaagtggtgagattatggccgcgagtctggccccagacctgcacctggtgagtcgcagtctgggtgccaaagtgaaggacccgctgttcggcggtccgccacaagattttttcgttcgtgaagccaagcgtctgtataaagacgccctgacggcgcagaaagatgacatgcgcagtcgtccggaaagtgttaaggccggtctggcggccatgcaactgctgctggccccacaaggctgtgtgaaagatgacggtccatgcctgagtggtccgatcaccgttacgagcaagaatggcaaaccggtgctggagggcggtccagtgctgggtgccagcctggccgagaacctgctgctgctgcatgttcaaggtctggatcacaaaaaagccggctggaccacggcgatcgacccggcgtttctgacgcgcgccctggccgtgcacgactacctgagtgacctgacgcgtaaacgcggtaagctggtggaagaaaagagtcgtgcgctggccggcgttatcgacgccttcctgcagggtcgcgatgcgcaactgccaaatggcgatagcatcggtccgcaaacccgttttctggcgtttgcgggtcacgacacgacgctggacgccctggccgcgcgttatggtctgagctggagtttcaaagatcagccgaccgaacaactgcgcacgcgccgttggctgagccgtgacggcgagagtcaagccgcccgtagttttatcgcgctgagttttagtaccaatccgtggctgcactgtctgaaggaaggtggtagtatgaagccgatggaaggtttccgcccatggtgccgcagtcgcgccgagcgtgcgggtaatagccgcctgagttgtcgtcgcgagggcatgatgcgctgcacgcaactgagtagttggcgcagtcgctga

SEQ ID NO: 188 - DNA, 1323 bp, Alistipes timonensis
  source 1..1323: mol_type = genomic DNA
atgaaacgcccactgctgattctgagtgcgtgtctgtgtgcgctggccgcggccgttagctgcagcagtggcctgcgcaccgccgacggtgagagcgtgccggatcgtgttatgggtgtgtatttgccgtatccggagaccgtggcgccaagtccgggtgccccagagggttacgagaccttttatatcagtcactatggtcgccatggcagtcgctatctgctgtatgacagccaatatgcgttcgtgcgtgacgttctgagtcgtgccgccgcggatggtaaactgacgccaagcggccagaaagccctggcggactttctggaggcctatccgcagtttgagggtcgcgccggtatgctgacccgcattggtgcggcccaacaccgtgccatcgcccgccgtatggccgaacgccatccaagtgccttcgccccggaagcggccgtgcgtgccagtacgagtgccacggcccgcacgcaggagagcatggaggccttctgtgcggccctgcaaacgtgtcgtccaagtctgcgtattacgtgcgccgaagacgccgccctgaacccgtacagtgcgggcagcggtattccgacggagtacgacctgcgtgtgaaaagtccagaagccgagtggcgtccagacttcgaaacgttctgtcgccagcaaatcgatgccgagcgttttgccgcccgcatctttacggacacggattatgccgcgggcctgtgtgacctgacggatctggaacgtggtgccttctacctggcggttcattttcgcggttgcggtattgcggccgattggctgcgtctgttcacgctggacgagctgtgtacgctggcggcctgtgacgcctatacgttttacatggaaaaaggtccagccgcggaaacgagcgatcgtacctgggccctgagtgcccacatcctggacaaaatgctgaccgacgccgagcaagatattgcggacggtacggccgccaacctgcgcttcggtcatgacggctgtatcatggcgctgctgaccctgatgggcgcggatggttggaccgccacggcggccacccaagaggaaattgcgcgcgcctgggacgttagtcaaattccgatggcctgcaatctgcagtggatcttttatcgtccggtgagcggtgagggtgagccactggttcgcacgctgctgaatgagcgtccactgcgtctgccggtgaccgacccggaaggtatgtgtgcgtggagtgatctgaaacgctatctggcggaacgctgcgacacggccttcgccgtgctgaacaaggaccgcacgccaaataacctgtga

SEQ ID NO: 189 - DNA, 1329 bp, unidentified
  source 1..1329: mol_type = genomic DNA
atgaaactgctgaacaaaacgaccttcctgatcggcctgggtctggttttccagacgatgacgattagtgcccaaaccctgacggaccgcgtgtatgaagacccgacgaccagcgcgaatatctaccatgtgtataaacaacgtgaaggcaaactgacgccagccccaaaaggttaccagccgttttatattagtcactacggccgccacggtagccgttatctgacgggtgattactacttccgcccggccatggccatgctggaggcggagaaagagaaaggcaatctgacggaggtgggtgagaacctgtatagtgacattaagaagctggccgccgcgcatgaaggcatgtacggtgagctgagtccgcgtggtgcccgcgagcaccgcgccattagtggccgtatgtataatcgcttcacgaaggttttcaaaagtaaaacgcgcaacaaagtgaattgtatcagtagcgttgttccacgctgcatcattagtatggccaattttaccacggccctgaacgataatcgtccggaactggagttcagttacaccacgggtgacaagtactttgagctgctggcccacaactacgatacggacgagatctacaaaacgattggccatattaccgatagcctgcgcaaggccacgtgtcactatgacaaattctatggtaagatttttaagaatccaaccgtggccgtggatagtatgaaaagtccgtacagcctgatcggtagtgtttatagtagcgcgtgtatctgcgagtgcctggacttcctgaacgtggatctgttcaaatatttcgacaaggacgaactggcccaacaggccgtggttcgtaataaccgcgtttacggtgatctgggtaatagcattgaatgcggtgatgtggttgccgccagtgcccgctttctgctgcaagatttcgtggacaaggccgatgcggccctgcaaaatggcagcgacgtggcggccgatctgcgttttggccatgacagtggtattcaaccattcttctgtatcctgggcattgagggccacgacaaacaattgcatattgcggacgccgataagtgttggtttagcgatctgaccgtgtgtatgggtaccaatctgcaaatgatcttctacaaaaacaaaaagggccaagtgctggttaaactgctgtataatgaagccgaggccatgatcccggcggtgcaggcctacagcggtccgtactatcgctgggaagacctgcgtagctactttgtggatcgtattgccgccgccaaagccttcgccgccacgctgccaccgccgcaaccgcgtgaagaatga

SEQ ID NO: 190 - DNA, 1335 bp, unidentified
  source 1..1335: mol_type = genomic DNA
atgacgctgcgcaagacgctgagtgccgccgtggtgacgatgctgacggcgtttgccctgacggcgacggcccaaaccacggccgatgcggatatcagtgccaaagaggcgattagtaaagacaacaatctggcgggttgcatgtatcgcgcctatccgggcccaaccaaacagctgacgccagcgccggagggtaaacgtccattctatatcagtcattacggccgccacggcagtcgttggctgattggtcgcgacgactataagtatctggtggaggttctgcaaaaggccgatagtgcgaaaaccctgacgaaatatggtcaggaactgctgaagaagctgcgtgtgatcaacgaccacagccgtaagcgcctgggcgagctgacgagtctgggtgcgaaacaacatcgtgaaattggcggtcgcatggccgatcgtttcccagaagttttccagggcggtgttcgtatcgaggcgcgcagtagtacggtggtgcgctgtattctgagtatggagaatgagctgctggaaatgatgcgtcgttacaataacctgcgcatctggagtgacgccagcgaccgcgatatgtggtttatggccggtggtcgtgagaatctgggcgacgtgaaaggtaaaaaagcggcccaagaactggagtggaagtgggagcagaccaaaatgccactgttcaaaagtatgttcacgaagatcttcaatgacacggcctatgtgaacaaaatggttaataaggataagctggtgtatcgcctgtacagcatcgccgtgaatatgcagagtctggacgaacgcaaagagatcagcctgtatgatctgttcgacaatgatcaactgtatgcgtgttatgagaaggaaaatgccggctggtttatcaattatggcagtaatctgtggacgcaaggtaagatgccgtacagccaacgttttctgctgcgtgacatcattacgaaagcggacagttgtatcgccctgccaaaaccgggcgccacgctgcgctttggtcatgataccatgatctgtccaatggtgtgtctgctgggcattggtggtcgcgacaaaatgctggacgacaccgacttcgataatatcgccgagaaatggcgtatgtatgaatttgttccgatggccgcgaatattcaattcatcttttatcgcagcagtccgagtgacggtgacgtgctggtgaaggtgctgctgaacgaagatgaggtggccgtgacggaagacctgaaaacggacatctttccgtactatcactggaaggatttcaaggagtactttctgaagaaactggacaatttccaactgcgttga

SEQ ID NO: 191 - DNA, 1392 bp, Desulfovibrio piger
  source 1..1392: mol_type = genomic DNA
atgctgccaagtcgtaaggaaatcctgatgcgtacgtggacgtgtctggccggtctgctggccctgctgctgggcctgatgccgtgcccggttagtgccgcgccaagcgcgtatcacctgagtaaagtggttatgctgacgcgccatggtgtggccgccccggccgatgacaatcaactgccgcgtattacgggcaaggcgtggccacagtggccggtgggcccgggtcaaatcagtccacgcggtgcccagctgctgacggcgcaatgggcgagcctgcgtgccctgtatctgcaagccgatctgctgccggcgcaaaagaccgaaagtcgcgttttcgtgcgcgccgataacacgccacgcagtcgtggtacagcagaagcattgttgcaaggtttgacaccaggtaccctgccagcctatgcggtggttaatctggacccggatccactgtttgagccagtgcaagccggcctggccatcttcgacccagcggaaacggcgctggccattctggaccacattaatggtgatttcagccagttttgggagagtctgggtgagccgatgagtctgctggaccaactggcgggtccgctgagcgccgaagcctgtaatagtattaacctgccgggcggttgtcgcctgagtgatatggttccaacgatcagtattatggacatgggccgtcgtgtggagatccgcggtggcctgggtctgggtagtaccctggtggatctgctggtgcaagaatacgcgcaatggccgcaacaagaagcagcatggggtcaagcaaatgcagcagcattgcgtaaactgctgccgatccgcagtgagattttcaacgcgctgcatcgtaccccactgatcgccggtatttatggtggcccactgctgcgcgacatggccctggccctgtatagccaacacgcggacccacgtctgaatgaagccaagtgcgcggtgttcgtgggcaacgataataatctggccgcgatcggcgccctgctgggcatcgactggcaggccagtggctttccaccaaacgcgctgccgccaggtacgattctggcgatggaactgtgggagggtccgggtaatgatagtgaagttcgcttccgtgtgtttatgcagagtctggacttcatgcacgccccgctgagtgttacgacggcgagcggctatcaaaccgccccagaaagttatggcccagcgctgctggaggcccacgtgacgctggatggtcaaaaagacggcccggttatgagtcgtgaactgttcgaacaacgtgttcgtggtgcattggaaggtcgtggtttgccaagaatcaccttcctgccagaaatggtgagcccacaagccccagcgccaagtctgccggttcaaccataa

SEQ ID NO: 192 - DNA, 1467 bp, Chitinibacter sp.
  source 1..1467: mol_type = genomic DNA
atgcgtcacctgttttgcctgagtgtgctgagtgcgctgctgacggcctgtggtggtgacattacagatccggcggcgacacctgcgccaacggcaacaccagttgctactgcgactccggctccaacggtccaaccgtataccgtgagcgccgttagctttgatgccgaaacggagaacgtgctgagtgcggcgggtgacgtggacgacccagcgatctgggttcatccgagccaaagtgccaaaagtctggtggttaccgccattaaggacggtggcatgcgcgtttacgatctgcagggccaactggttcaaagtattggtccaggtgccctgaacaatgacagcaagggtaaaagccgctataataatgtggacattgtttatggttttaagctggatgacaatagtacggtggacctggccattgccacggaccgcggtcaagacctgatccgtgtgtggaaaattgacccaaataacagtaccaccccgctgagtgacatcaccagtgccacgccactgcgcctgttcccaacggtgccgaccgaaggtagcgagagtgatgccagtaaggacgccgcgattgccgttagcaaacaaaatacgggctatggtattacgcacttcagtgacaaggccagcggcaaacacttcgttctggtgaaccagcgcaaacaagcgcgcatccgtcaatttgagctgattgccgaggcgggcggtaccgttaatgtggttgcggtggcgggccgcgactggcgcttcccatatagctggcgtggtcagaacctgcgcgaaacgaatgacctggacgcgacgcgtgattggagtccgcaatttgaaggtatggtggtggaccaacgcaatggcatgctgtacgccggtcaagaggacgttggtatctggcgcgtggacctgaagaccggtcaggccgacacccaaccggtttatgagacgcgtggtagtagccgcgaaacgtacacgacgggtaccccggccacggttgtgaagcatagtttctttaacccagagagcaagattagtcgcgacctggaaggcctgagtatttactatggtccgaatggtacgggctatctgattgcgagtagtcaaggcggtgcgcatggcggtgccccgaacgtgagtgacgcgccgtatgacgatagcttcgccgtgtttgccctgaatggcaacgcgaaaccagtgctgaagcaaggttttcgcctgagtcaaggtccaaatggcaacaccgagggcgttcaagaatgtgacggtgccgatgtggtggccctgcaactgccgggttacccgttcggtctgctggttacgcaggacggctataatgatgacctgaataatctggatggtgtgacggcgcaaaccaatctgaagttcacgagttgggagaaaatcgccaccgccctgaaactggagaaatacaataattttgatccacgtaagctgtga

SEQ ID NO: 193 - DNA, 1533 bp, Caulobacter sp.
  source 1..1533: mol_type = genomic DNA
atgattcatcgtcgccaacgtttggcagcagcaagtgcattggcattgacattgggtgcagttgcaggtcaaacctttgcgcaggccgttgccccagtgggtgccaccgttccgaccgcggaaggtggtgccaatgcggccgccctgctgtatgatcgcgcggacccaggtaaaagcgttatcgccgccaccggtgagctgggcggtctggaattccatgatctggacggtgcccgcaaaagcgcgctgccaggtggcgaaacctatggcgttgatgtgcgcgacgacgtgctggccgttctggatcgcaaggacggtcgtattcgcctgagtcgttatgactttgcgaccggtcaagccagtgccattgatgcccgtccgctgatgctgggttatgccggtgagggtctgtgtctgcatcgtagtgcccgtgacggtagtctgtacgccttcgccctgggtggcgacggccagctggatcaatggctgctgtttccgaccgccgacggtaaactggatggtcgcattgtgcgtcgcctgcacctgagtagtgaggcgaaatattgcgttgcggacgatgccagtggcatgctgtatgtggcgcagcaagccgttggtgtttggcgttatgacgcggatccggaggccgaagcggtggccacggttgttgacatcaaccgcctgggccacattgcgggtgaagtgggcggcctggccattctgaatggtggcgcgcagggtgattatctggttgcggcgaacgccgacgccggtgactataatgtttacgatcgtaatgcggacgaccgttttctgggtcgcttccgtattcaagccaatggcgcggacgcgatcgagggtccagccggtctgtttggtgttcgtgcaccattgggtgcagatttgccagcaggtgcattgttggttacagatgatcgtaaggttggcgccaatacgaaactgctgagctggcgcgatgtggccgttgccctgaaggttagtccaggtcaagttacaccaccagcagcaccaagtcgtttggcattggttaagccgacgatggaaacgcgtccggtggaacacggtggcgacgccgcggacgacccggccatttttgtgcacccgacggatccggcgcgtagtgttatcgtggccacggacaaaaaggcgggtctgtatgtttatgacctggccggtaaaccactgcaattcctgccggatggcaaaatgaacaatgtggacctgcgtggcggttttaaactgggtggccgcaccaccacgctggttgtggccagtgatcgcacgcataagagcatcgccctgtatacgattgacccagatacgcgcatgctgaccaatgttgccgacggtgtgcaagcgacgggtctgagtgacccatacggttgtgcctgtacgcgcgcggcccgtgtgcgcccgacgtgtagtagtgcgatcccaacggccgcgtgtgccagcggtaattggagcccaccgcgtccggcccgtagtgccccacgccgtagtgccacgtaa

SEQ ID NO: 194 - DNA, 1623 bp, Butyrivibrio sp.
  source 1..1623: mol_type = genomic DNA
atgaaaaagaccgttattacggcctgcagtctggtgctggcggccgttatcggtagtaccggctgtggtaaaattagtctgaagaatccaaatatgaaaaacagtgaaacgaatagcgagctgaagacggaggacaataccgaagttaagacggaaactaacaccgagacgaataccgaaaccaacacaggggccacgacggaagcgaaaacggaggccaagacggaggagacgaccgaagcgaagacggagacgacgaccgaagccaaagcggagacgacggccgaagataacacggaggcgaatagcgaagcgacgagtgaagttaatgccgagagtaacaccgagacggaaaccaatacgggtagtacccagaatatcgatgaggaagcgaacgccggtggccgtattatcgactacgacggttataagctggatcaagtggtgattctgagtcgtcataacatccgtagtccgctgagcggtggcgacagtctgctgggtaaaattaccccgcacgaatggtttaattggacgagtgccccgagcgaactgagtctgcgcggtggcgtgctggaaacgcaaatgggtcaatatttccgtcaatgggcggaagaggaggaactgattccaacgaactaccacccggataatggccaggttcgtatctatgccaacagcaagcaacgcacgattgcgacggcgcagtattttaccgccggtatgttcccaacggccggtctgccggtggagtatcatatggaattcgacaaaatggatccggtgtttacgccgcaactgacgttttggacggatgagtacgagaaagacgccaaggatcagatctatgaatattatgcggatgccatgaaaagcctgaatgataattacgagctgctgagcgatgttattgacctggaagaaagtaatggtttcaaagatcaaagcattacggccttcaccgcgggtgatgaggaattcagtctgaaagagggcgccgaaccaggtgtgagcggtagcctgaagacggcgtgcagtgttagcgatgccctggtgctgcaatactacgaggaaagtgatacgcaaaaggccggttttggtcaagagctgacgtacgaacaatggaagagtatcagcgaaatcaaagatgtgtatggtgacgtgctgttcacggccccgatgattgcggtgaatgtggcctacccgctggtgagtgagatctacgatgagatgaacaccgacggtcgcgtgtttacgttcctgtgtggtcatgatagtaatgtgggcagtgtgctggccgcgatcggtgccgaagactatgaactgccgaacgccattgagaaaaagaccccgattggctgtaaaattgtttttacgaagtggagtaaaggtagtgaggtgttctggagtgccgatctggtttaccagaccccaagccaactgcgcaatatgccaattctggacaccgatagtaacagtccggagatcttccacattagcttcaagaatatcaaacaaaacgaagacggtctgtataaggcggacgagtttatgaaaatctttctggacgccatggatagttacgaaagtctgtaccaggaatatgttgaaaaggcggcgtga

SEQ ID NO: 195 - DNA, 1650 bp, Desulfatirhabdium butyrativorans
  source 1..1650: mol_type = genomic DNA
atgaaaccgaagcgttgcttcctgatgacggtttgtgccctgctgattctgatcagtggtccagcgatggcgcagccgcaggccccagtgctgagtgtgaccacgcgcggctgggtgtacctgagttggaccgaggttagtggtgcgacgggctataccctgagttatgcccagattccgtacacgggtagtagtgccatcggtaccgttgatatgggtacgcagaccagtctgagtggtgccctggccgcggataccgccttctacgttgccgttcaaagtcgtgacaacaccggtattagtgagtatagtaatgtggtgagtatcgccggtgacagcaccacgctgaagcaaattatcatttttggccgccacagtattcgtagtgcgaccagcgacccggccagtctggcccaatacgccgtggacgcgtatccggaattcacgggcgtgccggccggttacctgaccccgcgcggccaacaggcggccacgctgctgggcagttatttccacgagtatctgctgctggaaggtctgctgaccggtgaggcccaaacggatattagtcgtagctacttccgtgccaacagtattcagcgtagcaacgtgacggccgccaaatttggcagtggtctgattccgggcgcgacgatcccagtgcacagttacaaaattgcggatggtgccagtagtgccgaaccggatccagtgttcgacccgatcttgacaaaagttgcaacagttgatccagcacgtgcattgggtgaagttcaaggtatctttaatagcggtgcggccatcagcagtgcctacagtggcgagctgagtctgattcgtagcgccctgtatcccaagggtacgcaaccaacgccaggtgcggcccacggcagtaccgacccgacgagtcaaacgagccacccgattaccctgaaagccagcacgagtaccctgtacacgggtggtgtgattgatctgggcggtctgaatctgaccaacagcgcggccgacccgttcgttatgcagtacacggataactttccgctgaatgacgtggcgtggggccgcctgagtctggatgccgttagtcaacaaagccgcatcatcaatctgatcttcaatatccaaatcctgccaccgtatctgaatcaagtgcagagtagcaatgccgcccagcatgtgctgcgtacgatggagcaagccgtgacgggtcaaaatatcagtggcgcctttggtgacgcgcagacgcgcgtgcatgtgattgtgagtagtgacggttatgttatcggtctggccggtctgctgcacctgcattggctgctgccaggctaccaaccggacttttgcccaccaggtggtgccctggtgttcgaggttcgtcaagtgaatggtacgggtgaacatgtggtgcgcgtttattacaccgcccaaagcttcgaccaactgcgtaacctgacgccgctgagcctggccaatccaccgcagaccatgaacctgctggtgccaggttgtagtcgcagtgccacgaatctggatgtgccattcagcgcgttccaacaagtgctgcagaatgccattggtatggagtacgttcaagatccaagtaaagagaccccaccaggtgtgctgacgggcgttccactggagtga

SEQ ID NO: 196 - DNA, 1671 bp, Cystobacter fuscus
  source 1..1671: mol_type = genomic DNA
atgtttcagggtgttctgctgaccgagctgctgctgggtgcgcgcggttgcagtagtacgatgcgctgtccgcgccgtctgggtgccgccagcaccctggaagaattcatgagtgccaaacgccgtgttctgggtagtgccagcctggcgctggttctggtgccactggccagcctggcggaaccgccagtgattccgccacgcgccgagaccccggttctgcaccgctatgacgaggccccgcgcacgccggatagcgacgacccggcgatctggatccacccaagtcgtccggaacgcggtctggtgatcggcgttctgaaggaggccggtctgcaagtgtacgacctgagtggccgtgttgtgcagacggttctgccagccaaccgtccggccatcagtgccggcgacccgatggcgccaggtccgcgtccagaagccgcgacgagcgcctgcccggaaagtgaaagcggcgagacgtttggtcgcttcaataacgtggatattcaatacggctttccgctgcgtggtgcggacggccgtgttcgcaaggtggatctggccgtggtgaccgatcgtggttgtgatcgcctgcgcatttacgccattgacccgggccgcgcgggtggtccactgttcgacgttaccgcccgcgccgcccgtcgtgttttcccagagcgctttgtgcgtccgagtccgttccaaccgacgggtgaaccggagggtgtgcgtccgaatccactggatgaccagaacacggcgtatggcctggccctgtatcgtgacccaagtaatcgcttccacgccttcgtgacgcagcgtagccgcgccgtggtggcgtggctggagctgtacgaagccggtaccgagcaagttggttatcgtgaagtgcgcgaatttcgtttcgacagtcgttttagtttgccacgtccaggtggtggtggtgcattggcatggagtccatgtcgtgaagaaccagcagaagatccacaattcgaaggtctggtggttgatcagcaggagggtattctgtatgccgcccaagaggtggtgggtctgtggaagatcccactgagtgtgagtctgccgcgtgtggttgacgtgccacgtggccgtctgattgagccggtgaaaagttttggtgcgccgtactgggccgttccggacgatggtgagtatgcctgtgagctggaagccccagccccggcgccagagggtacgattgccgttccgggtaacccggccgtgggcggtaaacatctggaagccgacgcggagggcctggccctgtactatgcgggtgatgagaagggctatctgattgttagcagtcaaggtgatgacaccttccacctgtacgaccgcgaaggcggttggacgcgtgaacgcggtaatcgccatctgggcaccttccaagtggaaggtgtgggcgagacggatggtcatgacgttgtgaatgtgccaatgggtgccggcttcccacgcggtctggttgttctgcaaaccggcaaagccccgccaccaccgagtaccgaaccagtgaatggctatgcctatgatggtagtacgcgctttaagctggttcgctgggacgacatcgccgaagccgtgccgccgggctttaaagttgataccgacgattaccacccgcgtgatccgcatgacgattaa

SEQ ID NO: 197 - DNA, 1911 bp, unidentified
  source 1..1911: mol_type = genomic DNA
atgatgatccgtgtgctgagcaagctgctgctgccgtgcctgctgtgtggcctgctgaccgcctgtgccaccagcccactgaaagcgacggataagctggccctgccgccgccaagtgccgcgtggaaaaatattagtgccatcacgaaaaaaggtcaaccgctgtggctgatcaccagtgagacgacgggcctgagcctgatcgacagtagcaataatcgcctgctggcgctgcagggtcactttgagctgctggatgttcgtccggcccacgacaacgaattctgggcgctgaccgtggatagccaaagtaatgccccgcaactgctgctgatgcaaacgcatccagagattcgtctgcgcgagcgtcacaccctgttcaatggtgatttccagatcgacggtctgtgcctgtataaggacgcccaaagtcacctgatggcctattttctggatggcaatggtagcggtgagctgcgctggctggtggatggcagtagtggtaaggttgtggatacgagtatcaccatgctgaacctgccaccgaatagcgaacattgtacggttcacgacgccagtaacagtctgttcattagcgaggcggaaatcggtatttggcgctacccagcgaatccggaaatcgaaaatagtcgtattctggttgatcgcacgttcgacggcggtaagatcgatggtgaggtgatgcacctgacgacgacgcgcaatgcgctgtatgccgcgacggaacagcgtctgaatatctatagtccagcccaatggcaactgcaaagcagtgtgccgtttccgccaggtgtgaccgttgagggcattgcggccagtcaaagccaactggccgtgctggacgaagcccagcaagccctgacgctgatgccgctgagtcaaccaacgagttatcgcagcagtgaaaaacatatcctgccggttgtgaaggccaatgcgcaaacaacagcaatggatcgtcgtggtgatgcagcagatgatccagcaatttggattcatccacaagaaccagaaaaaagcctgattctgggtacgaataagaagtggggcctgctggtttataacctgcaaggtgaggccgttcgtagtattgcgacgggtcacattaataatgttgatgtgcgccaaaatctgctgttcgccaacgagccaatcgacatcgccgtggccagtaatcgtagtaataacgccattgccctgtataaaatccacccagaaaccggtgacgttaacagttgggccagcctgccgacggatctgaatgatgtttatggcatttgtctgttcacgccgaccccaagtgacatttatattattattaacgataagagtggtcgcgtgaatcaatataaactgggcagccgcaatcgcaagccgcacgccacgctgacccacagcgtgcaactgaagagtcagccagagggttgcgtggccgacgatacgcgccgtgaactgttcatcggtgaggaggataaaggtgtgtggcgcttcaacctgacgaatgacaaactggttgagccggagctgattgcgaccacgaacggtcatctggtggccgacgtggagggtatcgatatcgtgaaaaatgaattcggcaactacctggttgtgagtagccagggtgatcacagttacgcggtgttcgaaaataaagccccgtatgaattcctgggtaagtttcgtattgccccgaatgcgaccctgaaaatcgacggtaccagtgaaacggatggtctggcggtgacgggcatgccactgggtaataactacccggccggtctgatggtggttcaagacggtcacaatctgatgccaaatgccccgcagaatttcaagctgatcagttggaaggacgtgctgaacgcgctgaagattccaaccgtggaataa

SEQ ID NO: 198 - DNA, 1935 bp, Microbulbifer agarilyticus
  source 1..1935: mol_type = genomic DNA
atggcgctgaaaaccctgccgaagctgtttagtattctggccgttgcggccctgtgtagtgcctgtggcccacaaatcggtaacgatgccgccaaaccgaagggcattgccagcgagcaccaactggccctgacggatgtggttagtagtcaactggccccactggccctgggtggcgtggaatatctgctgctggccagtgaaaagcgtggtctggttctggtggacaaggagggtagtgagaaactggcgctggacggcggtaccgttgaacgcttcgccctgcatccgctggagcaagaccgctggttgattgcggtgtatgatgaagacaatggtgagctgcagctgcgtctgctggatgtggaagagggtagcccgcgcatccgttacctggccgcgatgccgacgaacgccccgcaagttgccatgtgtttcagtagtcaagcgggtcgcacccacctgtttgcgatcgacgaaacgggtctgggccacgagtatgtggtgcacccacgcgagcaggcctggaccttcacgggtctgcgtccgctgtactttggcgagcaggtgagcagttgtgttgtggatgatcgccgcggtaaactgctggtggcccaaccgccgctgggtatctggagtctgaacgccgacgccgaaaccgatgaggcccgtcaggtgttcattgcggccagcgcgctgccggacggcgaatttggtggtctgtggctggatgaggtgaacggtaatctgtggctgaccgcggccgaaaaggttatggccttcgacattaacgatccggccaaaggcccactgtttgtggaggccctggccgacattgaaccagttagtgccgccgtgcaaggcgaagccctgctggcgctggaggaagagggtgaccaagttcatcgcttcgccgtgagtctgccgcagccgccagccgaagttcaagcgttccgtggcccggttgagatcccacgcgtgcgtgccagtggtcagacggccccagttgcgagcggcggcgacgccgcggatgatccagccatctgggttaatccggccaagccgagcgccagtctgatctttggtacggacaaaaagagcggtctgagtgtttatgatctgaacggcaaactggtggagcacttcgaggttggtcgtgtgaataacgtggatctgcgtccgatgcaacacggcaaattcgtggccattgccgccgccacgaatcgcaccgacccaggtgttagcctgttcggcattaccgccgcgggcgccgtggaatacctgggtctgcgtaatctggatatggaggatccgtatggtctgtgtgtgtatcgtaaaggtgccgacctgatgacgtgggtgagtgataaggaaggtgccgttcaactgctgcaaattgttccaggcacgggtaatgtggattggagtctgcgtaagctggcgaccctggaagtggcgagtcaagtggagggttgtgtggtggacgatgaaatgcaaatgctgttcttcggcgaggaagacggtggcatctggcgcctggacattgcggcctatctggcgggcgaggccaagccgcaactgatcgcgccagtggatggcgaacgtctggccgcggacgtggaaggtatgggtttttaccatgccgcggataaaagctatctggttgtgagcagtcagggtaacaatagttatgccctgtttaaccgcgacggcagtgagttcgttggtcacttcaaggtggatattaatctggacaaaggtctggacggtagcagtgaaacggatggcctggaagtgagtagtgccgccctgggtgcgcaatacccacaaggtctgctggttgttcaagacggccgtaaccgcatgccgagtgccagccagaacttcaagctggttagctgggccgacatcgccgagacgctgcaactgccatga

SEQ ID NO: 199 - DNA, 1968 bp, Acinetobacter nectaris
  source 1..1968: mol_type = genomic DNA
atgaacattatctttctgaaaaaaacctgtttcgtgagcattgtgctgctgggtctgatgggttgtggtcaaaagacgatgccagtgccagaaaagagtgttatcagtaaagacggcacgattcaatttgaaagtattagtattaatggcaccaaagatgtgttcaaggttcaaagtgcgaagctgctgagcatcccgtattggagtgaagcgcatctgattcagacgagtaaaacgcagggtctgcaaatcctgaaccgtcagaatcacgtgctgcaaagtattgatggcgcctttggtgacattgactaccgtatcaaggataatctgctgttcattcagagtgtggacctgaaacaacagcgcccaaccctgctgatcttcgatatgaagagtaagcaatggcagccaagtatcctgctggaaaaaaccaagttcaagattgcggtgtgcctgtatcaagaccagagtcagcagctgtacagttttgttatcggtggccaaggtagtgcccaacaatgggccgtgaacaccacgccaaatgccaaacagccgatgcaactggttcgtaatctgaacatcccaattggtagtaagagctgcgtggtggaggaccaacaagaacgcctgctgattaatgaggagggcatcggtatctgggagtatctgacggatgccgagcaaccgtttaaacgtaagatcgttgacatggtgaaaccgtatggtcatattgagggcagtccaggtgcgttcacgcaggttgataatatgctgttcgttgtggacacccaacagccgttcatctacaaatatatccacgacggcaagaattggcaaatcagcgatcaactgaacaccgccgagctgaaaaagccggaacaactgagtgcgaaacgcgagggcgagaaagttcaactgctgctgaatgataatcacaaactgaaaatcgccagtctgccatatacgaccgcccaccaggacaagaaaaaggacgaacactttcaattcgtgcaagcgaaagtgcagacgaccccggttccaaacgtgggtgacgccgcggatgacccagccatttggtacaatgaagaaatcccgacggcgagtcgtatcctgggcaccgataagcagggtggtctgcaagtttacagtctggatggcaatgagcagcaatatctggccgttggtcgtctgaataacgttgacatccgtccaaattttaattgggatggccaacgcgtggatctggccgttgcgacgaaccgcgatcataatagtctgcatctgttcgcgatcgaaaagaaaacgggtcacgtgagtgaaattggccaggcccagacgagtctgaaggatatctatggtctgtgtctgtatcaaaacaaacagggtgacatttatgccatcccaaatgataaggacggtaccttcattcaataccatctgagtgccctgaagaacaaagttaatgccaaggaaatccaaaaattcagtgtgaaaacccaaccggagggttgcgttgttgatgacaagaatgaccagatcttcctgggtgaggaagatcgtgcggtgtggcaaaagagcctgctgcagaataatagtaacctgcaagaggttgccacggttggcaaaaatcaaattcaggacgacattgaaggtatgggtctgtatcatggtaaaaagcaaagttacctggtggtgagcagtcaaggtaacaatagttatgcggttatggaagcccatgccccatatcgttaccgcggtagttttaagattgttatgaacagtgtggaacaaatcgacgccgttagcgagaccgacggcctggacgtgacgagccataatctgggtggtgtttgggaaaagggtatgctggttgtgcaagatggtcacaaggtgatgccagaggaccaccagaatttcaaatacgtgccatggagtgagatcagcgaaaaactgaatctggaagagtga

SEQ ID NO: 200 - DNA, 2010 bp, Catenovulum agarivorans
  source 1..2010: mol_type = genomic DNA
atgcaatattttcaccaactgacgcacaacaagctggcgaaagtgagtgttgtgattaccgcggccgttttcgccctggcggcctgccagagcagtattgagacgaccgcccatagcgccgtgcaggcgctggccaaagccccgcaacaaatctttcaacatgacaagctgaacggccaaaaggttatccaactgaaccaagacacctgggtggcgaatagtgaaaatttcggtctgatgctgattaaccaacaaggtaaaatggtgcagaattatgccggtaatttcgagagcctggatatccgccaactggacaataagacgtggtttgttagtagtgtggacaaagaggcgggccagtttctgctgttcaaggtggtgctggccgcgaatgcccaaacccataaaatcacgctgctgaacacgattaagccagaggccagcgtgattgataatcaatgtctggcctatcagaacggccaactgagcagtttctggctgacggcccagcaacaagtggaacaacgtatcgtttatctgaatcaaccgaatgcgcagccaagtaatgtgctggtgcgcagttttagcgccccgccgggtgcgagtgcgtgtgtggttgatgacgtgaaacaacaggtttacgtggccgaggaaagcgccggcgtgtgggcgtatccaacgaatccggagaaagaactgagtcgtacgccgattgccctggtggcgccattcggtcagattgacggtgaaatcaaggacatcagcctgctgatcgacggcagcctgctggtggccctgccggaagcggagcaaattcaacactatcagacggataatagcagccacctggcccaagtgtatagtgtggcgggtttcaccattgaaagcatcgacgcccaacagaccgccgataacaagtggctggccaacttttacgatgatgaaagtggcgcgtattatagtattgagctgccgctgatggtggccgataaaagtatcgccaaacaaggtgccagccagctgacggccgacatgcagacggcgccagttgcccgcttcggcgatgcggccgacgatccggaagtgtggctgaatccgaacaacggtgccgatagtctgatcctgaccaccgacaaacgttatggtctgaatgtgtacaatctggccggcgaactggtggagacgattgccagtggccgcatcaacaatattgatatcagttatggtgcggaattcaatggtgacaaatttgatttcgccaccgccagcaatcgtacgtataatagtatcacgatgtacaagatcagccataccggcgttatcagtgagctggccaatctgccgacgagcctgccagacgtgtatggcctgtgccaataccaaagtccgattacgggtgactattttgttttcatcaacgacgaaagtggtatctaccaccagtaccaactggactttagtaccaatcaagttaccggcaagctggtgcgtgagttcaaagtggccagccaaccggaaggttgcgttgccgatgcgcaaacgcaaacgctgtatctgggcgaggaggattggggtatttggaccattggcgccgagccagacgccggtacgcgcctggccccgttttaccaagttgacggtgacgtgctggttgatgatgtggagggcctgagtctgtatctgccaaacaagagtgccgccaaaaatcgtagtgagaaatatctggttgccagtagtcaaggtgatgacagctacgtggtgtttgatctggccaagaatgcgagtcgtggccatgttgtggccaaattcaacatcgttgccgacgttcaaaaaggtattgacggcgcgagtgagaccgacggcctggccgtgatcgcggccccgctgggtgatctgtatccactgggtgcgctggtggtgcaagacggtcgtaatattatgccggttcaaccacaaaacttcaagctggttgattggcgcaaaattagtggcctgattatcaaacagtga

SEQ ID NO: 201 - DNA, 2211 bp, Corynebacterium timonense
  source 1..2211: mol_type = genomic DNA
atgcgtaaatttagtggtgccttcgtgatcagcctggcggccgccgttattgcccaaaatgccagtgtgagcagtgcgcaagttgccgactacagtgcggaatttagcgcgcgtacgacgccaagttttacgggtagtaacaccagttatgccaatagcaatgaggcgagtaagctgagcacggagctgcaggaagccccggatggcttcgagccaagttatagcgcgctgttcgcgcgccacggtagtcgtacgagtagcgacagcggttattacgaccgcctgattaatggcctggaagccgccggtcgtgtggattttttcggcggtgcgccgacccgcaccgagctgggcaaagaaattgaagatcacctgcgccagctgcgtcaactgcatgatcaaattggcggtggtaacctgacgggtctgggccgtgaggaaagtcgcgaactggcggatagttttgcggagcgtaatcgcgagctgctgagcgatagtgaggccaaactgcgtctggaggccagtaccaaaggtcgcagtattgaaaccggcgagattttcgcgcagcaactgatcgcccatcgcttcgttgtggcccagattccagaccgcgcgacgcaaaacaaagaagttctgcaaaaccaagatgtgccggagagcccagcgcagcaacgtcgcgaagcctacctgcaaacggacccaggttatcaggccgtgaagggtatcccagagaattggacggccgaagttcagccgtacgcgaacgccgtgtttgaacagctgcaaggtagtcgtaaaccaagtttctatggttactgtggtggtcgcgcccgtgatctgttcgaagcccgcgcgtttagtgagctgatgagcgttgaacgcgaagccgcgggcgttggcccgtttccagccattccgggtgccgaagacatgtatcgcagtgtgcaacaatgtacggccatcgacgagttctataagcgcggcgtgggtctggagggccaagatcacagcaacgcgaatggtctgccactgctgaaccaaatttttgccggtattgacgcgagtgagaacaacgaggaagttggtcagctgctgttcagccacaatgaaatcctgacgccgttcaatgcggtgctggccattccggagattggtgccaatcaaaaatttcgcccaggtgaactgtttaactatgagacgtatccggtgctgaaccaagcgcaaagcgacccaatgatgggcaacatcgaatggaacgtttttaagcatgaaaatggcacccgcctggtgcgcatgcagcacaatgagcgagccgtgcacttcggtcgcagctgcaaaccgatcaacgaagagttcgactatttttatctgtacgaagatctgaaggcctgtctgccggacgccggtcaaagtatcccgagtgaggacgaactgagtaaggcccgtaaactgctgcaagaggcgctgagcgaggaagagctgatccaaattgttagtgtgatcgaaaatgaagatggtagcacgaccctgaaactgagtaacggtcaggttgtgacggtgccaccgaatcgccagattgtgggtacggaagagaccggtagcgccgttatcattaagctgagtgatggtagtgtgatcgagatcgcgaaagggccaaagggggaacccggggagcagggtcctaagggtgaaccaggtgaacaaggcccgaagggagagccaggggagcaaggcccaaagggtgagcctggagagcagggtccgaaaggagaaccaggcgagcagggcccgaagggagaaccgggcgaacaggggccgaaaggcgaacaaggagaaccgagccgcatcgttgcgcaaggtgtggacggtgaaggcaataccgttctgacgttcagtgatggtacggttgtgacgattccgagtggtgcgctgagtattgaacgccagtatctggacagtgagggcaacaccgtggtggagttcagcaatggtcagaagatcgtgattgccagtccagccccgagtaaaagtagtagtggcagcagttggagcgacagtagcagtctgacggcggttggtagcagtgccttcgccgcgggcggtgtggtggttgcctttgtgctggccattgcggccgtggttggcggtctgctgggcatcatcccgctggcgccgccaattcaaagtctgatcgacctggtgcaacgctaa

SEQ ID NO: 202 - DNA, 2217 bp, Alteromonas macleodii
  source 1..2217: mol_type = genomic DNA
atggacaaacaaaaaattacgcgtatctatccaaccatgggcaagaccttttttctgaccggtatgctggcgaccctgtttagttgcgccaccgcgggcaaagccagtcatcaaccgcaagccgatatccaagaaccggttctgaagctgaagcagctggcgaacggtttcagtcaaacgctgtattatcgcagcgacaatcagcaaaccgcctacaaagatacgcatgccaatgagcaagagacgccaaaagccctgaacaagagttataaggacacgcacgccaacgataatgaaaaactggccccggcgctgaatgcccaaaccaatgacgacacgcacacgattgaactgcagctgaatgagcaccaaggtatcagtatcagtaatagccaaggtaaggcgcgttttattgcgggtagtttcgttctggccagcgcggagattgccgaattcgagaataaacaatgggtgagcatggtggtgttcaataataatacccaacacatcgaagcctaccgtctggccccggacacgctggatatccaacaacaactgacgcaaagtgccaaaggtagtgaggcgatttgtgccgcggttaccgcccagggcgcgccacatatcgttaatatcgatgccaccggtacgctgaatcaattcgagattcacgataaccaatttctgccgctgcgcaatttcgccatcggcccgggtatgaaaagctgtagtctggacatgctgacccacagtatctatctggccgatgaattcgccggtgtgtggaaggtgaacgccgatattgaaagtgaactggaaaaagaactgttcttccataatgcgaacatcgccatcgaaggtgttagcacgctgagcagtaatacgagcctgtttaagggtagtctgctgggttgggtgagcccaaacaaaagtggtgtttggctgcagagtcaatgtcagacggaatttatcacgctgcaatatgacctggcgggcgtgccgcaaaccattgaaccagagttcattcacctggccttctatgaatacgaaaagggtccgcgcattagcgtgatcgtggacgatgacgcgagtggtaacttcttcagtgccgagctgagtagcgaactgagcgccgcctatttcaatcatggttgtctgagtaaagatgaagaaccaatcgcgaacgccggtagccgcagtccgaagtatgccctggcgcgtgttctgccaagtgccgaaacgcaaccggtgaagaattatggtgacgcggcggatgacccagccatctgggttaacgcgcagagcccgagtcaaagccgtattctgggcaccgataagaaaggtgccctgaatacgtatgacctgctgggccaccagcaacagagtctggcggtgggtcgtgttaataacgtggacgtgggttacaatgttgccatcgcgaacatcaataacagtggcgagacgaccttcacggacatcgccatcgcgagtaatcgtagtcataatagtctgagtgtgtttgaaattgacgagttcggtgtgctggcccacctgggtgaaattgacacgacgctgaccgatatctacggcatgtgcctgtttgttgaaaacggtgtgacccaagttttcgccaatgacacgagtggcctgttcgagcgctatgaagtgagctttaataagaataaaaaggccaagggcacgctgacgcagagttttagcctgccgagtcagccagaaggctgtgttgtggacacccacacgaacacggcctacttcggtgaggaaggtgccggtatttggagcatggacatcaacagcacgaaacacgaaccagcgttcgttagtgcgatcgccccgccagtggaagcggatgttgaaggcctggccctgtttaccgtggatgcgcaaacgtatctgattgccagtagtcagggtaacaatagttatgccatctataacatcaatagtaaaaacgccgacaaactgaccctgatgggtctgattcgcatcacggccgacatgaataacagtattgatggtgtgagtgagaccgacggtctggaggccacgaatgccaacctgggcggtgtgtacagtgaaggtctgtgggttgtgcaagatggtcgcaatgtgatgccgagtgaaacccaaaatttcaagctggttagtggcgcgcgtctgaaggaggccatccgcaacctggtgagcaccaagcactga

SEQ ID NO: 203 - DNA, 2376 bp, Clostridium sp.
  source 1..2376: mol_type = genomic DNA
atgaaaaagctgttcaaaattctgttcagcatttttattgccatcattctgagtttcaatttcattacctattatccaaaggccagtaataacctggagctggttcaagatagcacgaatatcacgaagattccgaaaaactttcgtaagagcacggacgtgctgaatctggacgaaggcgagaaaatcaacctgaccggtctgaaggaactgaatattagtggtagcgcccaattcagtaaagaaggcctgagcctgattaagaagagtatcggtgacaaatacaacatcacggttgtggacctgcgtgaggagagtcacggctttatcaacggtatcccgattagtttcaagaataagaataacaatgccaatgaaggtctgagcctgaatgagatcctgaatgatgaagcgaagcgtctgaacagtattaagatcaatgagccaatcacctttagtaacacgaatattagtgtgatcccgaaggaagtttatagcgaggaaaccctgaccaaaaataataacctgaattacatccgtattccggtgacggacggtggtctgccaagtaccgacatggtgaaccgcttcaaggagaccgttatcaataacaaaggcaataataattggttccactttcattgtaaggccggtattggccgtacgaccacgttcatgatcatgtatgatatcgtgaaaaactgtaatgaagttagtctggaggacattatcacgcgtcaggtgaagctgtttgagatgagtgagaatgatagcaagggtttctatagtggtgatcgctataaattcctgagtgacttttataatgagtgtaagggtaacagcaagaatatttttaacaagaataacaataagaatgactattacatcaagaacaatattatgccaaagaccctgtatgttattaaagataacgaaatgacgaaggaggagcaaacgatggttgccaccctgcagggcaacgtggccacgaaaagcaaagaacagatttacatcctgagtaattatgaaccgagttatgagacctggctgaatgatctgaaagagaattataatgttaaatacaagttcattaaagacccatatgagctgctgagtaaatttaaagattacatcaagggttacattctgtataataatagtaacaaatatagtattaacaatggctgtagcctggcgagtctggaaaacggtatcgttatcgatgagctgatcgaagaaaaggtgaaagaaattggcgtgaagaacctgatcaaagactgtcgtgacaccgacaagtattgggccttcaataacctgtggaataaaggtctgaatcacagtacggttattcagctgagcccggacaaaaccatggccctgcgcgactacgccatcgtgagtaaaagtctggttttttatgaggaagagattaacaacaaggaattccgcgagaaaatttttagtagcatggatgacgttagtcgtgtttttggttggggtccagacgaacatacgaatgtgagtattgcgagtaagttcggcgttgatatcgtggccagtgactggagttacaacctgagtgtgctgagtagctatccgacgcgcgatcaaaagcagaatctgagtaaagagagcgttaaggacaatgcgcactacgtgacgtttatcatgagtgacggtgataatcaacaatggttcctgggcagtaacttcaatagcaataactggtatggtagcaaatatcgcggtaagttcaatctgggttggagtatcagtccgagtatttattatctggccccgacggttttcaacaagtactatgaaagtgccagtaatattcgctacaaagattattatctggcgccgccaagcggtaacggttatatgtatccgagtaagtttccagaggaaaaattgaataactatacgaaacgtctgagtaattacatgaagaaagtggaccaaaactatgtgctgattctggatgacgagagtttctatcgtaccgatctgtgggacaaatatacgtgccataataatattgacggcctgatttatctggactataagaaaaataataactataatggtgaaatcgtttggagtaataataaaccggttgtgagttgtcgtgacctgatgtggaagggcctggaggaagaggacaccctgatcgaaaacattaatagccgcgttaaagaaggttatacggacattaagaatccgaagagttacacgtgcgtgtatgtgcacgtttggagtaaaaatatggatgacctgaacctggtgatcgaagaactgaataagaacagcaaggtggaagtggtgaccccgtacacgtttatgaagctgattaaagagaatatcaagaaatga

SEQ ID NO: 204 - DNA, 2466 bp, Clostridium beijerinckii
  source 1..2466: mol_type = genomic DNA
atgaacaagaatttcaagcgtattaccatcctgaccctgctgattgttagtattttcagttttgccgttaacaataatgccatcgcccaagttccaagtgataaagacaacggcgtgcatctgattctggacagtgttaattataacagtgtgctgccgaaacacttccgcaagacgaccgatctggccgttgttaaagacaacaaggacctggatctgaagggtctggataaactgaatattagcggcagtcaacaatttagcggcaacaatatcgacctgctgacgaaggcgattgatacgagtctgccgatgacggtgatcgacctgcgtcaggaatgtcatggtttcatcaacggttttgccgtgagttgggcggacgcccgcaataatgccaacgtgggtctgacccgcgaccaagttattgcgaaagaaaaggaagacctgaagagtatcaagctgaatgagccaatcacgttttacaataacagcaaacagacgatggatgttaagacggttcaaagtgaagaggagctgaccaagagtaagaaactgggttatgaacgcgttacggttcgtgacggtggtattccgacggatgacatggtggattacttcatggagtttattaaaaataagccaaaagacagttggctgcacttccactgtaaggagggcatcggtcgcacgagcacgtttatgattatgtatgatatgatcaacaactacaaagacgtgaatgccgacggtatcatcaagcgtcaactggcgctggccaacttcgaagacaccacgctgaaaagtttttataataatgaacgcatgggcttcctgaataagttttatgattactgcaaagcgaatggcgatagcttcgacaccaaatggagtgagtggaatagcaaggccagtagtattgacgaaacggataaaagcaatgcgatccaagtgagtaacgtgaatagcgtgtatatgaagaacaatattgttccgaagagtctgtatgccattagtctggacagcatgaccccaggtgagcgcacgatgatcaccagtctgcaaggcctggtgaacaatcactgtagtttccaaatctataccctgaatagcagtcagccagattataagatttggctggaagatctgaaaagcaatcacaacgtgccgtatgagatgatcagtgacccgtgggaactgctgaagatttataaagattacattaagggttacgttctgtataacggcgaaacgaaagatccaagtattaataacgcgtgtagcctggccggtgtgaaggacgcgattgttatcgatgagaaactggagccgaagattaaagaactgggcattgagagcaaaggtgactgtcgcagtaccgacgagaattgggcctatgataatctgtggaacaagggtctgaatcatagtatggtgatggaactgagtccggaccgcattgatgccctgcgtgactatgccatcatgacgaaaagtctgattttttacgaaggcagtgttgataagacggccctgcgcgacaaaatcttcagcagtgttgatgacaacgccacctgtctgggttggggcccagatgaatttatcaacgtgagtacggccagcaaatatggtgtgagcatggtggcggccgactggagctataacctgacgacgctgagtgcgttcaaagaaccgcacattaataaaaacagcagtctgaaggttccgaaggaggagaatgtgcattacgtgacgttcatcatgagtgacggtgacaaccaacaatggaacctgggcacgaactatggtagcagtaaatggttcggttacgagaaccgtgataaactgagtctgggctggagcatgacgccagccctgtattacctgagtccgaccgtgttcgacctgtataataagagtattagtaacgaagatccacgcaataattttattgtggccccaagtggtaatggttacatctatccgagcaaattcccacgtaacaagctgggcagctatgttgacctgctgaataattatatgaaacgcgtgaatgagaactgtgtgagtatcattgacgacagtgcctttcacgatatcgagctgtggaatgaattcacgaagaaaagcaatattaaaggtctgttttacatcgactaccatcgtcatgacaattataagggtgagattctgtggagcaataacaaaccactggtgagttgccgcgacctgctgtggaatagtctggaagatgaagaggagctggtgaagcgtatcaacgaccgcgtgagtctgggccagacgaatatccatagcccggaggcctacagcttcgtttatgtgcatgtgtggacgaaagaaaccagcaacgttgagaaagtgaccaagatgctgaaagagaataaaagtgtgcgtgtggtgccaccggagacgtttatggaactgatcagtaaaaatgtgaagcattaa

SEQ ID NO: 205 - DNA, 882 bp, Bdellovibrio bacteriovorus
  source 1..882: mol_type = genomic DNA
atgaaaaagctgctgctgctgccagttctgttcgccgtggccgcgtgtgccgaaaaaacggttagtctgaccccagataagccggttagtaccaagatcccgtttttcatgacgcgtccacagccgaccacgccggtggaactggtttttgacaatgatcacgcgacggccaagccgatgaactaccgtaagaatgatagcctgcgtatgagtggtagtgccaccttcagcccgaaagcgctgaaggaggtgagtaagccagttaagaaaaataaagccagcctgtacgtttttgacctgcgccaagagagtcatggcctgattaatgacattccggtgacgtggtacgcggatcgtgactgggcgaacgccgacctgaatcatgaagaggcggtgcgccgtgagcgtcgcctgctgggtgacctgcgtgttggtgagaaaatcggcacgaccacgatccaaagcattgaaaccgaggagagtatgattcgtacgggtggccaccaatatgtgcgcctgaccgttacggaccacgtgcgtccagttgacagtgaggtggatcgctttatcgagagtgtgcgtgccctgccggaaaatgcgtgggtgcatttccattgtcgcgcgggtaaaggccgtaccacgaccttcatggtgctgtatgacatgctgaaaaacgccaaagccgacagttttgaggcgattatcaaacacaatacggaactgagtaatgattatgacgttctgaccgttccggcggacgagaaggattggaaatatccgtatcaaaaggaacgtgccgcctttgtgacggaattctacaattatgcgaaggcgcacccaaatggcgagggtatgctgtggggtgaatgggttctgcgctaa

SEQ ID NO: 206 - DNA, 906 bp, Clostridium tyrobutyricum
  source 1..906: mol_type = genomic DNA
atgaagaagaatcgtgtgcgcatcggcatttttctgctgttttttattttcctgctgggtgcgagtaacgtgaatgttctggccctgcagaaaaacaccgcggacaacaagctggagctgacggttgatagccacaagaaagcggaaatgccaaagcgcttccgtaagaccagcgataaaattaagaccgagggtaaaatgccgaatctgacgggtctggcggaactgaatgccagtggtagccaacagtttaccccgggtaacatcaacctggtgaaggacgcgattggcaaggacaaaaaaattagtattgttgatctgcgccaagagagtcatggttttatcaatcacctggccgtgagctggaagctgccagacagtaacaaagccaataagggcctgacgcgcgaacaggttattaaagaagaaaagaaactgctgaaaagtattaagctgaatgaaccgctgaagatcaagaataaaaccgttatcccagtgaaggttcaaagtgagaagcaactggtgaaggacacgggtatggattatattcgcgttaccgtgaccgacacggagcgcccggaagatgacatggttgattattttgtgaagaaagtgcgcaaactgccggaggacacgtggctgcatttccactgtaaggcgggtatgggccgtaccacgaccttcatggcgatgtacgatatgatgaaaaatagcaaaaaggtgagtctggaagacatcatggagcgccaggaactgctgggcggtgttaagctgctgaagccggttggtggtaaagagagtgagagtcagaagcgcagtgacttcctgcgtcaattttatcagtacacgaaggaaaataatgataacttcaaaaccagttggagccaatggctgaatagtaaatga

SEQ ID NO: 207 - DNA, 909 bp, Acidaminococcus fermentans
  source 1..909: mol_type = genomic DNA
atgaagttttggaaaattacgacgggcatcctgctgatgagccaactgagtctgggtctggcctgggcggacgcgccgagtattgccgccatcgttaagccaagtaaaggctatgtgtggcgtctggacacgaaaaataagctgcaactgccccgcaactaccgcgccgatgttgagaattgtatgagcggtagtgcgcaaccgagtattctgggtctgagtagtctggttcaagaactggcccagcaaggcgttaagccgcaacaaatcatcctggtggatctgcgccaagaaagtcatggcttcgtgaacggccaggcggttagctggtatggcgacaataactgggccaatgtgggtaaagccgacgcggcgattcgtaaagacgaggccaatcgcctggcgaaaaccctgggtaaggaaacgagttattacaagctggataaaaacaagcaaccgcactttaaaggtaaggagaatgttgccgccgcgctgacggagcgacaagccgccgcgagtttcggtctgggttatgcccgctttgcgagtaccgaccatatttggccggagccagaagaggtggatgcgtttctggcgtggcaaaaaaccctgccaaaggacgcctggctgcacttccactgccaagccggtaagggtcgtacgacggcgtatatgatcatgcgcgatatttggctgaatggccaaaaagacagtctggaaaccatttgcgcccgtcaacacgcgctgggtggccaagacgttctgcatatgacccataaggaagcgtggcgccaaacgatcgatgacaataaggtttaccgtctgaaacaattctatacgtatgtgaagggtctgcaacagggtaccattacgggtacctggacggaatatctgaaacaaaatccgtga

SEQ ID NO: 208 - DNA, 924 bp, Desulfovibrio alcoholivorans
  source 1..924: mol_type = genomic DNA
atgaccccactgtgtagtgcccgcttcctgccgcgcctgctggcgtgtgccctgtttctggccatcctgggcgccgcgaaaccaggtccgggtgccgatggcccagttctgatttatgactatccaccgggtcaagcgctgccgatccatttccgtctggccgatggtggtccaaagggcgatccggcgagcgccaagctgcgtctgagcggcagtagcgagtttgacgccgcgggtctggacaatctggcccgcaccctgccgggcccactgacgattgtggatctgcgccaagaaagtcatggcttcctgaatgaccgcccggttagttggtttgcgccaaaggatgaggccaatatgggtaaaaccccggaagaggcggcccgcgatgaagcccagcgcctggcggccctgagtaaagccggcacggccgcggtgaccgttatcgaggcgaaaagtaagagcggtggtattaaaaaggcgcgtacgctgcgcgtggacgttgttggtgttgcaagtgaagcacaattgacagcagcaagagcagttggttacctgcgcctgttcgtgacggatgacatggccccagatgcggcgcaagttgaccgttttgtggaatattgccgtagtatggccccgaatacgtggctgcacgttcactgtcacgcgggcgatggtcgtacgacgacgtttatggtgctgtatgccctgctgcataatccggccggtcgcagtctggaggagatcgcggccgatcaggcggccgcgggtggtgttgatctgattggtcaaccgcgcaagggttggaaaaagccactgtacatggcccgtgttgcctttctggaaagatttgcagcatatgcagcagcaaatccaggtggtgcaccaatgaccttcagcgcctggaccgcgacgcacaaatga

SEQ ID NO: 209 - DNA, 954 bp, Desulfovibrio frigidus
  source 1..954: mol_type = genomic DNA
atgagcgccaaaattttcttcccgatcaagacccgtatcctggacgatatcttcaaactgatcctggccagtttcgttattctgctgatttttaacagtaatagcttcgccgacgatagtgtgctgattctggacgcgccggccgttgaaaatctgccgcgcaacttccgcaccagtgcgtttccatttaagaatagtacggatagcccaagtcgtacgggtctggacaccctgaaaatcagcggtagtggtcaattcagtgccgaccagtttgacgtgatgcgtagcaacctgccggccacggtgaccgttgtggacctgcgccaagaatttcatggtaatctgaatggcgcggccgtttgctggttcagttaccgcgatagcggtaacaaaggcctggatagtgcccaagtgaccagtggtgagcaagaagccctgagtctgctgggtgcgcagaaggagattcaactgggtatgaatatcatcaaaaatgacgatggtggctttaaggttgacaaaaagtaccagctgaaaccgcacacggtgtatagcgaacacgagctgatgggtgcccgtggcgttggttacatccgcattgcgtgtaccgatcacctgcgtccgccggacgttgccgtggagcgcttcgtgaagtttgtgcgtaatctgaaaagcgatgtgtggctgcacttccattgtcgcgccggcaagggtcgtacgaccacgtttatgctgatgtatgacatgctgcgtaatgccaacaaactggacctgggtaccctggccgcgcgccaaaagatgattggcggtagtggtctgctgggcaaagttagcacggataaggagtggaaaattgagatcggtcatgaacgtcaaaatttcatccgtaagttctatgactacgcgaaagccaacccaaatggcctgccaatgacctggctggagtggcaaaagtga

SEQ ID NO: 210 - DNA, 960 bp, Clostridium acetobutylicum
  source 1..960: mol_type = genomic DNA
atgcacaagaaaaccaagctgatctgtatcggcattctggcgaccgccatcgtgacgattagcatttataacttcgatttctttaaacgcaccccactgaaggagcataaagtgaaactggtgatcgacgccgaaaataagaatacgctgccaccaaaattccgtacgaccagtgacacgattagtctgcataagaagggtagcctgaatctgagtggcctgagcgacctgaacgccagtggtagtggcgccttcagtgaggatgaactgaaaagcatcaagaataagattggtaacaaaccaatcgtggacattgacctgcgccaagagagtcatatttttgttaatggcatcggtatcagttggtatggtaaaaacgacgatgccaatctgaatctgaccagcagtgaagtgctgaaggacgaaaataataaactgatgcgtattagtaaggataaaaaggtgacctttgacaaactgagcaaaaagaagagtatcagcaacatcagtcaactgaatgacgtgaagagtgtggagaccgaagaacagctggccaaggcgctgggcatcaattacagccgcatcacggtgccggaccacaagacgccagacgatgcccaaattaacagtttcgttagctttgttaaaaatctgccgaagggtacctggctgcacttccattgtcgcggtggcaaaggtcgcaccacgacctttatggcgatgtatgacatgatgattaatagcaagaaagttagtttctacgatattatgaagcgtcaaaaactgattggtggtgcggacctgctgagtggtcaggatagcctgggtaaaagtgacgccgagaaacgcgtgcagctgctgaaaaagttttacaattattgtcaaaataacaacgataatttcaaaacgagttggaccaactataataataagtaccgcatcaaaaatctgcagcgttga

SEQ ID NO: 211 - DNA, 975 bp, Allisonella histaminiformans
  source 1..975: mol_type = genomic DNA
atgaagctgcaatggatgaccaccctgctgctgacgagttgtattagtctggcgggctattgcggtagcggtgcggccgaaatgcacagtccaattccactgcagcaagaaattccgccagcgccgggctactttaatggtcgtctgtggcgcctggatgccagcgccagtagcccatttccaagccattttcgcaccagtgcgagtgcctttgacctgagtctgccgcacaaactgccgctgccgagccgcaagggtctggatcgcctgcgtattagtggcagcgcccagccaagtggtatggccatcccggccctggcgcgccacctgaaatacctggccagtggtggcccagttttcctgatcgatctgcgccaagaaagccacggtttctttaacggtaacgcggttagttggtatggcaatcgtaattgggccaaccgcaataagacgacggaggcggtttgtcaagacgagaaggaacgtattcagagcgcctgtggcacgcaggtgacggtttatagcctgaatgaagaaaaagaaccggccgacgcggagaagatcaccgtgcaaagtgccagtaccgaagaggccctggcggcccgcgccggtctgcattatgtgcgtttcccgattacggatcacaccacgccgagcgaagcgacgatccgccagttcatcagtttcatgcgtagtctgccgccggacgcctgggtgcactttcactgtgaggccggtatgggccgcaccacgaccatgatgattctgtgggacatttataacaatccggaactgccactggaaaccattgttgagcgccagcatctgattggtggtgcccaaatcagtctgtatccccaagaagagcgcaaaggtccgtggaagtttaacgagagtcagagccgcgcgcgcattattcatatgttctatgatacgatgcacagtcagagtgccagtcacgccgatcgtgcctga

SEQ ID NO: 212 - DNA, 987 bp, Centipeda periodontii
  source 1..987: mol_type = genomic DNA
atgcacgcctttaaacatctgctgacggccctgctgctgtgtaccctgtgtatcattccatataccgccagcgcgacggacacgaacgcggactaccgtggttatgtgtggcgcattgatacggccgcgggtaatgaagccgagctgccacacaactttcgcaccgccggcagcccgttccagacccgtaccgacacggcgaagttcggtgtggatccgaattataccccaagtcgtgaaggcctggatgccctgccactgagcggtagcgcggaattcagtgttccggccttccatgcgctgctgaaagatctgcacacgcgcgccaagggtagcatttgcattgttgacctgcgccaagagagtcacggtttcatgaacggtaatgcggttagttggtacggtaaacatgattggggcaatatcggtcgtacgaagtatgaagccctgtgtgacgagaatatgcgcattcgtagcgcccagggcaaggatgtggtgctggcgcacctggataagaaaaagcaaccgaaaaatccacagaccatccgcgtgattacggccatgacggagcgcgaactggtggaggatgccggtgttcgctacgtgcgtctggccgttaccgaccacaaatgggccgacccgcaaacgattgataattttgtggacctggtgaagaagatgccagcggatacgtggatgcactttcattgtcaggccggcaaaggtcgtacgacgagtttcatggcgatgtatgacatgatgaagaacccgagcgtgccactgaaggatatcctgtatcgccaatacctgctgggtggtgcctatctggcctatgacccaaccacgctgcatgccccgaccggctgggaggatgccgactatcatcataaggccgagatgattgcgaaattttatgattatgttcagcaaaatcacaaaaatgactatgccatcccatggagtatgtggctgaaaaagaacccgtga

SEQ ID NO: 213 - DNA, 996 bp, Desulfovibrio magneticus
  source 1..996: mol_type = genomic DNA
atgccaaccctgccgccaccgtgtcgccgtctgcgccgtagccatcgcacaagattggcagcaccatggccattagcaggtttgttggcagttgcattggcattgtttctgacgggtccaatcgcggtggcctgggccgaaccggaaccagatgttggtgttttgacattggatgcagcagcagcaagtgcattgccacatcgctttcgtacgtgtttctttccgctggcgcgcccagatggtgccaccaccccgagtcaggagggcctgaacggtctgcgcatcagtggtagtagccaattcagtctggcgggtctggcgattatgcgcgaacaattcccgccacgtgccgttatcgtggacctgcgtcgtgaaagtcatggctttctgggtgatgcggccgttagctggcgtctgccggacaatcagggtaatccaggtcgtgaagcagcatttgttgcagcagcagaagcaggtttgttggcagcaattgatgaacgtccagacattgtggttgcccgcgaggcgaaacgtggtggcccaacgccgctgaccctgggtccgctggccgcggtgaatgaagcccaagccgcggcgagtctgggcctgggttacctgcgcctggccgttagcgatcacacgcgtccagacgatgccgtggttgagcgcttcgtgcgtttttaccgcagtctgccgccagatgtttggctgcatttccactgtcgtggcggtgcgggtcgcaccaccacgtttatgacgctggtggatatgctgcgtaacgccccagccgtggcgttcgaggacatcattgcccgtcaaaaggccctgggcggtagcgatctggcgaaaacaacaggtggtagtgcaccaggtcgtgatgcattggcacgtgaacgtttggatttcctgcgtcgcttctatgattacgcccgtgccaatccaagtggtgcaccattgggttggcgtgcatggttggcaggtggtgcaaagccgtga

SEQ ID NO: 214 - DNA, 1194 bp, Burkholderiales bacterium
  source 1..1194: mol_type = genomic DNA
atgaaagcgaagatctgtggcagtctgtgcctgctgagtgccgccgtgctgctgagtaacgcgcatgccgaaacgccaagcgacgttctgcaagccatcccacacaccgaggaactgctgaacgtggtggtggttagtcgtcatggcgtgcgcagcccgacgcagagcgccgagaagctgcacggctggagtgacaagaaatggagtgcctggccagttgcgccgggtctgctgacgagccgtggtttttacctggtgaaggccacgtggaaactgaatcgtagccgtgcgccatttacgtacggtgtttgtccgaagccagaagacgtgcaaattattgcggatgttgacgagcgtacgcgcaagacggccgaagccctgaatgagggtctgtatccaacgtgcggctacaaagttaaggtgaccagcagtgaacatagtgccattttcagcccgctgaaggcgaaagtgtgtcgcatcgattatccaaaggaactggaagaaaaactgacgcagaaggtggtgggtatcaatgagaaattcgccgcccaaatggccgaaatcagtaaactgacgggtcacgaattcaccggtccgatgcgtgccaaagtgagtaaatacaaggtgggcttcaagggcgccccgtacgactgtagcagtatcacggagatttttgccctggaatggggtcagaatccagaaaaaaaggttgcgtgggatcagctggactggaacggtatcctgcacctgatgccgatccgtgtgagtgttttcagcgccctgaatcgcgacatggaagtggcgcgctataagggcagcgcgctggcgaacaaaattattgagagtctggatcacggtccgaaatacacgtatctgattggtcatgacaccaatctggccaatctgggcgaaattttcgatctgaattggaagctgccgggccgcgaccaaaatgaaaatacgccaggtggttatctgacgtttgagaagtggagcgttaatggccaaccggaaatccgcgtgttctatagtgccctgagtccggagcaaatccacgcggagcgcgtgacgcaaccgacggacgactttgaaattctgccgcgcggtaccaagttcgatgagtggaagacgacctacggtcgtcgtctgcaaagtaattgtattgcggacgataaatatgaaaaccgcaagtaa

SEQ ID NO: 215 - DNA, 1275 bp, Acinetobacter nectaris
  source 1..1275: mol_type = genomic DNA
atgatcaaaaataagctgatttgtgcgatcctgaccagtattaccgccctgacgggtacgacggtttatgccgcgaccaatgattaccaactggaacaggtggttacgctggttcgccacggtattcgtccacaaaccaatctgcaagagctgaatgacgccacgggcaaggattggccgaaatggagcgttccagatggttatctgacgcagcgcggttacgacggcattctgaatcagagccaatatcagctgaagacctggcaagaggccggcctgccgatcagtctgaaccaagagaattgtagtaatcaaaatcaagtgtttatttgggcggcgccagaccagcgcacgcgtaagaccgccgatgcgatcgccgaaagcattagtcaaacgtgtcacccggaggttggtgtgagcggttataaacatgatccgctgttcgatgccctgaagatgggtagtgcgaccgaccaattcgacgagattaagaaagaattcaaggcccgtatcgtgagtcaagatgacatccaggcgaaatacgcccagagcaaccaacatctgaaagcgacggtgtgtgcgccaaatggttgtgacttcctggacaaagactggaaactgaagctgaacaagaatggtcaaccgaagctgaacggtccagccgacgagggcagtaatattggtgaaacgattcgcctgcagtatagtgagaatttcccactgagtcaagtggcgtttggtcatgtgaagaatgccaatgatgttaaaaagctgatggttttgcacgacgccaaatacaaatatctgaatgaaatcccgctgttcgcccaacgcggcggtagcattctgtaccaacaaattctggatgcgctggataatagtaaggccaccgaggcgccactgcatcgtccgctggttgtttttgttggtcacgacacgaatattagcgagatcaaaaccctgctgcaattcaattggcagctgccacaatatctggccaacgacatcccgccaggcggtacgctgagttttgaaaaatacaaggagaaaagcacgggccaatattttgtgaaaattggcttcagtgcccgtaccctggatcaatggcgccacctgacgccgctgagtaaggcgcaaccgctgcacgaagacgttctgaaatacagttattgtaaaaccaccagtgttggcgtgctgtgcccgctgaatcagttcctgcaaaatgcccagaagcaaatcgtgcgtctgaacatcgaccaaccactgtacaagtaa

SEQ ID NO: 216 - DNA, 1296 bp, Dyella marensis
  source 1..1296: mol_type = genomic DNA
atgcgtccaccaggtagatttgcaagagcagcattgttgggtgcagcattgttggcaccagttttggcacaagcagatgatgcaacgctgcgtctgcgcgttgtggttctgcgccatggcgtgcgtgccccaacgaaggccccggatgccctggcgccatatgccagtcgtccatgggagacgtggccggtggcgccgggtcagctgacgccacacggtattgagggtatgcgcgccctgggtgcgaattaccgtcgcaccctggccgccgacggcctgtggagtggcgcctgtgaccgtatggatcaactggtggttatcgccgacagtaccccgcgtaatcatgccagtggcgcggccctggttcaaggtctggccccagcctgcgacggtacctacctggccctgccgacggatcaaaacaacccactgtttcactttggcggtaaagatgacgacaaggatgaggacgataaaccagcattgccagttacatggccaccagcagttttggcacaactgcaacgcgtgctgctgggctgcgagggtcaagcctgcctggcgaaagcccatgccgacggccgcaagaccctgctggacgccgcggacgatgccgcccgcgcgaaagccctgaagacggccggtagtctgagtgaaaatctgatgctggaatatgcccaaggcttcccaagtgcgcaggttgcctggggtcaaggcgacgaggcgaccatcggtcgtctggtgacgctgcacaatctgcagtttgccctgagtaagaaagcgatgccagccgccgcccgcggcggtagtaacctgctggcccacgttctggccacgctgcaacaagcagatggtgaaaaaccagatgcagcagcattggcaccagcaagtgcacgtgcagttttggttgttggtcatgataccaatctggcgcaactggcgggtctgctggatgtggactggcacgacgccgcccacccagacgactatccgccgggcggtgccctggtgttcgacctgtggcaaacgcatgacacgtacagtgtgaccgtgagtacggccatgccaacgctggatgccctgcgccgcgccgatgttgcgcgtccaaccgccctgacgcgtaaaaccctgcgcctgacgccgtgtccggtgacggaccattgtccactggacaagctgagtagctggctgcgtggccgtctggatgcgacgcgcattgacccggcgctgccggacatgcgtgcctggagtgcgagtagtcaagaggcgccacatgaaccagcggttagtaagtaa

SEQ ID NO: 217 - DNA, 1299 bp, Escherichia coli
  source 1..1299: mol_type = genomic DNA
atgaaagcgattctgatcccattcctgagtctgctgattccgctgacgccacaaagtgcctttgcccaaagcgagccggaactgaaactggagagcgttgtgatcgtgagtcgccatggcgtgcgtgcgccgacgaaggccacccaactgatgcaagatgtgacgccggacgcctggccaacgtggccagtgaagctgggctggctgacgccgcgtggtggtgagctgattgcctatctgggccattaccaacgccagcgcctggttgcggatggtctgctggccaagaaaggctgtccgcaaagcggtcaagttgcgatcatcgccgacgtggatgagcgtacccgcaagacgggcgaagcctttgccgcgggtctggcgccagattgtgcgatcacggttcacacgcaagcggatacgagtagcccggacccgctgtttaatccactgaaaacgggcgtgtgtcaactggacaatgcgaatgtgacggatgcgatcctgagtcgtgccggcggcagtatcgccgacttcacgggtcatcgccagacggcgttccgtgaactggagcgcgttctgaattttccgcaaagtaatctgtgtctgaagcgtgaaaagcaggatgagagctgcagtctgacgcaagccctgccgagcgagctgaaagtgagcgcggacaatgttagcctgaccggcgcggtgagcctggcgagtatgctgaccgagattttcctgctgcaacaggcccaaggtatgccggaaccaggttggggtcgcattacggacagtcaccagtggaacacgctgctgagcctgcataatgcgcagttttatctgctgcaacgcacgccggaggttgcccgcagccgtgccaccccgctgctggatctgatcaagaccgccctgacgccgcatccgccgcagaagcaagcgtatggtgtgacgctgccaaccagtgttctgttcattgccggtcatgacacgaacctggccaatctgggtggtgccctggagctgaattggacgctgccgggtcaaccggacaacacgccgccaggtggcgagctggtgtttgaacgttggcgtcgcctgagcgacaatagtcaatggatccaggttagtctggtgttccaaaccctgcagcaaatgcgcgataaaacgccgctgagtctgaataccccgccgggtgaagttaagctgacgctggccggttgtgaagagcgtaatgcgcagggtatgtgtagtctggccggtttcacccaaatcgtgaacgaggcccgcattccggcctgtagcctgtga

SEQ ID NO: 218 - DNA, 1311 bp, Anaerovibrio sp.
  source 1..1311: mol_type = genomic DNA
atgcaaccactgaactatacgattaaaaagtgtctgggtctgctgggcccagcggccatcagtgcgctgctgctgctgagctggagtacctggacggaagccgcgagtgaactgaccgataagggttaccagctggaccaaattgtgatgttcagccgtcacaatctgcgtaccagtattacgagcgaagacagtagtgccgccacgatgatgacccatccgatgccgaaatggcaagtgccgcgcggcaacctgacgatgaagggtggcgttaatgagaccattctgggtcagtattttcagctgtatctgatggatgaaggcttcatggcggatagttggcaaccgggttacggccaagttgacttctatgccaacagttacgaacgcacgatcgccacggcgcgttattttgccacgggtctgctgccaacggccaatattaccatcaaacacaccaaggccgtgggtggtagtgatgccgttttcaataaaccgtttgccatggacacgccaaaactgatggagctggcgatggagtatcataacgagtacttccaaagttataaagatgtgtttagcaagtgctacgacagcttcgcccgtgttaccgactttccgaatagcccgtatgccaaaaagcacggcattgaccacattaatacggatgacctggccctgacctttagtggtaaggagaagaatgtgatcaattacgacggtaccgcccgcaaggcctataaactgctggatccaattattatgaattattaccaggaagccgataacagtaaggcggacttcggtaccggcctgacccgtaaggactgggagaatctgggtgccgtgaagacgaagagcatcgaaatggtggttggcaatccagccgtggcgctgagtcacagtcacgacattctgaacatcatggaaagcgacctgaaaaatgatacgctgaagtttgccttcatttgtggtcacgacaccaacatctttacgattctgacggccctggagaccgaggattacagtctgccggagacgatcctgaccaaggcgcccctgggcgttaaaattgtgttcgagaagcgccgtaatccgcagggcgacctgtttatctacccatacattgcgtatatgagcgacgaccaacagcgcaatagtcgtcagctgagtctgcacgaaccgccaatgatctacccgctgaatttccgcggtctggcgaagaatggtgatggcctgtaccgctatgaggatattctgaccctgatggaccgtgtgaacgccaaatatgaggagtattgcctgcattga

SEQ ID NO: 219 - DNA, 1311 bp, Avibacterium paragallinarum
  source 1..1311: mol_type = genomic DNA
atgaagaagctgacgctgagtctgaatctggttagtctgctgagtggtagcctgttttacccaaccaatctgagtgccgcgccgaacgtgccggataatcatcaagcccaagccctgcaactgaagaaaatcgtgatcttcagtcgtcacggcattcgcaccccactggtgggtaaagagagtattctgagtcaggcgacgccatatcgctgggttaagtggcaagataagaccggttacctgacggccaaaggcgcccgcctggagaagctgttcgcggcctattggagcgaatggctgcagcaaacgggtattctgagtagtcaatgtccaaaggatgatctgttcatttacaccaatgcgcgtccacgcacgattgacacgggtgtgagttttgccaaaggtgcgtacagccactgtgccatccgcgtgaattatctgggtgaatataacaccatggataataccttcaacccagttattcgcagtaaggtggataaagcgttcgagcaaaaggcccgtaaagccgtggatgaactggtgggcgaaggcggtttcgcgcaactgaaccgtcatctgcaaagcaattttaatgcgctgagtcaagccctgaatcaaggtcaaagtccgctgtgtcaggagaagcaactgtgcaatctggaccaagaggccaatacgctgacgttcacccaaggtaaagagccgaagacgaccggcgcgctgcgtgatggtacgggcgccgccgatagtttcctgctgcaatattatgaaggctttccggcgaaagacgttgcctggggtcgtatcgacaatcaagaaagttggaaaaagattgtggacatcaaagagcgctacaaccagctgctgttcggtacgcaagtgatggcgaaagaagcggccacgccgctgctgaccttcatccaaaacagttttaaggattacggctaccagcacccatatattgaaaaggcgcgtaatgcgaaggtggtgctgctggttggccacgacagcaatgtgggtagtctgctgccactgctgaaagtgaaaccgtattatctgccggatcaactggagaccacgccgatcagtggtaaggtggccttcgagaagtgggtgaatgccaaaggtgaagccttcatgaaggtggaatatgtttatcaaaccattgaccaactgcgcaacggcacgaaactgagtctgaccaatccgccgaaccgtatcacgctgcagctggaagactgtccaacgaatgcgcaaggtctgtgcaagatggatgacttctataatgccgttaagaacgcgctgaaatga

SEQ ID NO: 220 - DNA, 1317 bp, Cronobacter sakazakii
  source 1..1317: mol_type = genomic DNA
atgaaaacgctgttcctgcatctgttcctgttcatcttcattttcatgccaggtgtgttcccgctgcacgcccaaggtgccgataaaatgaaactggaacgcgtggtgatcgttagccgccatggtgtgcgtgcgccgaccaagtttacgccgctgatgcaagagatcaccccgtacccatggccgcaatgggacgttcccctgggctggctgacgacccgcggtggtgagctggccagtgagctgggccgctatcaaaagcgcgttctgctggataaaggcatcctggaaagtcatggttgcccgagtccggaacaagttgcggtgattgcggatacggaccagcgtacgcgtaaaaccggcgaagcctttctggccggcttcgccccaggctgccaaaataaggtgcactaccagaaagagctggataagaaggacccgctgtttaacccagtgaagatgggcgtgtgtagttttaacgtgagcaaaacgcgcgaggccatcctgacgcgtgccgagggtaatatcgaacgctatacgcaacgttacgatagcgcgttccgcaccctggagcaagtgctgaatttcagtcagagtgccgcctgcaagagtccgcgccaaccaggttgtacgctgacgggtgtgctgccgagtgagctgagtgtgagtcaggacaacgtgagcctgttcggtgcctggagtctgagtagcaccctgacggagatcttcctgctgcaagaagcgcagggcatggcggaagtggcctggggtcgcattcatggcgagaaacaatggacggaactgctgcgtctgcacaatgcgcaatttgacctgctgcaacgcaccccggatgtggcccgcacgcgcgccacgccactgctggacctgatcagtcgtgcgctgattagtaacggtagtaccgagagtcactatggtatcaagctgccagtgagtctgctgttcatcgccggtcacgatacgaatatcgcgaacctgagcggcgtgtttgccctgaattggagtctgccgggtcagccggacaataccccaccaggtggcgaactggttttcgagcgctggaaacgtgtgagcgacaataccgactggattcaaatcagttttgtttaccagacgctgcaacagatgcgcgaatttaagagcagcagtagcagtagtctgccacataaaattgtgctgccactgccgagctgtcaagagaaaaacgcggaaggtatgtgtagtctgaaacgtttcaatgacattgttcaaacgatccgcgtgccacaatgtgccgttatggcggagaaggaccaatga

SEQ ID NO: 221 - DNA, 1362 bp, unidentified
  source 1..1362: mol_type = genomic DNA
atgatcatgctgggtagccgcaatctgctgaccgtttttctgttcacgttcacgagtctgagctttgcgcaaaccaatgtgattgccggcagtaaaacggcgtatcagtataatagcaaccaaaccctgacgctggccccaacgggttaccaggccttctatatcgatcatgttggtcgtcacggcagtcgctacattagcaagagtaagtacgaagacctggcgtatcatattctgctgctggcggacagccaacaccaactgaccgacagtggtaaagatctgctgcgccaggtgacgatcctgaagcaactgaatcagaaccattatggccaactgaccaatctgggtcgtaaagacattagtctgatcagtaatcgtatgctggagaacaatccgacggtgttcaagggtcaaaaaattgaggttatcagcagcagtagcccgcgcgccaaggagaccgcggagattttcatcgatagttttaaggcgaagtatccgaatatccacgtgatccaacagccagacaatgaacaaacgctgctgcgtttctttgaatacagtccggcctatagcgagtataagaaaagcaaagcggttaaagatgccgttaaaagtattgagtatgccagccaaaccaagcaaatgagtaaacaggtggccaagggcatctttaaaaagagtttcctgatcaaactgaagaaaggtctggacgaaagcgaggatacgctggttaagacgagcgactttgtgattgccatttatcaactgtatcaagaactgcaagcgttcagcccgcaagtgctgaccgataaccacctggacctgggccgctattttagtacggaacaaaaaatttggctgaacacggttgtgaccgccaagaactatctgcagatcggtccagccttcgatgccacgggcattcaaattaagatcgcggccccgctgctgtgggacatgattcatagtgcggataaagccatcgttgataataatgtggacgccaatctgcgttttgcccatgccgaaaccgtgagtccgctggcgacgctgctggaaattgaaggtagcgcgaacgtgaccaaaacgctgttcaattatccaaccatctggcgcgccgataagattatcccaatgggtgccaatatccagtggattttctataaaagtaagcaaacccatcaaccgatcctgatcaaagtgctgctgaacgaacgcgaagttcacctgccgattaaaacggacagttatccgtactaccgctggaataatgtgaagcaattctatgttaataaactgaataagctgggcctggacgagaatcaaagcgccatcgaaatgctgaaaaacctgaaataa

SEQ ID NO: 222 - DNA, 1635 bp, Corynebacterium singulare
  source 1..1635: mol_type = genomic DNA
atgcgtatccgtaccctggccctggcgaccgccattgcctgcacgctgacgccggttcaggcgctggccgccgatgacaccacgtactatagcaccaagcaacattacgaaccgcaaggtagtaactatagtacggccccaacgggctttcatcaaatctatacgagtacggttaaccgccacggtagtcgtggtctgagtggttttaaatacgatgacctggcccagcaaatgctggagtatgcgaaagagcatgatcaactgaccgagctgggcgaaaagctgatcccgcaagtggaagcgatgattacggttaataaggaactggcgggtggcccaggtcaggaagccggttatggcaatctgaccgttgtgggtcgcgaggagctgcaaggtatcggtcaacgtaatgcgcaacgcaacgccgccctgatggagagcattgaagaggacaacctgaaagtgaagtacatgagtagcggcgcggatcgtgccaacgacagtggttggagtttcggtgaagcctggctgagccataatagtaaactgagcgacaatctggtggatggcatggaagacggccacgtggccattgagacccgcacggatctgctgcatgcccacaaggataaaaagagtccgagttacgagcgttacagcaagtggaaagacagcgagacgctggatagcaaagttcaggaagcctatgcgaaaccagccagtcaaaccgcggcccgcagcctgctgaacaaaatctttacggaggacttcatcgccggtctggaagacggcaccatcacgtttgttggtcgcgacaataagggcaagagtgtggagggtattgtggaagcggcgctgcaattttataacctgtacattattgccccggcgctggcgcatgaagagcagacgccaagcgaaggttggatctttgaccaatatatggacaatgccagtagcccgacctttgcctatctgctggacgtggaggattattatcaaaaaggcccagccatcgagggtcaaaccgttgcctatgacaattatgagccactgctggaggaaatgatccaaggcgttaaagatcgtgccgaaggtggcgacgtggccgcggagtaccgcttcggtcacgccgaaacgattatcccactggccgccctgctgaagctgccgggtagtgaaaaaggcacgccagccgatgagctgtatacgtgggagaatagcgagtggcgcggtgataaggttgccccaatgggtgccaacattcagtgggacgcctttcaaaacgaacgcggcgagaccctggtgcgcatgctgtacaatgaaaaagaaatcgcgttccatgacggttgcgagccgatcgagacgggcagtaccttctataccattgatgaactgagtgactgtctgccactgggtgccacgagtgaccacagtaaggcgcgcctgcaagaagacaaaacccatgagaccgcccagccaagcccgagtgatgccgttagtagtaaggccaaagtgtggggtattgttgcagcagttttgggtgcattggcaattgcagttggtgcagcaaccgccaatgcccaacagatcaaggacattctgaataagtttggtatccacgtgccgttctga

SEQ ID NO: 223 - protein, 417 aa, Pseudomonas putida
MSMKTLLIATLVLGAGAAAQAVANDDAQVRLGEYLARAGDCVACHTAKGGKPFAGGLPMETPIGTVYSTNITPAASGIGQYSFEDFDQAVRRGIGKDGSTLYPAMPYPSYARVSEQDMQALYAYFMKGVAPVEQANKASDIPWPLSMRWPLAIWRGVFAPEAKPWQASATADPVVNRGAYLVEGLGHCGACHTPRALTMQEKALSAADGEQFLAGSAPLEGWIAKNLRGDHKDGLGSWSEAQLVQFLKTGRSDRSAVFGGMSDVVEHSMQHMSDADLTAIARYLKTLPPSNPDDQLHVYDKQVADALWKGDDSKPGAAVYIDNCAACHRTDGQGYTRVFPALAGNPVVQTADATSLIHVVLAGGTVPATHSAPSNFTMPAFGWRLSDQEVAEVVNFIRSSWGNQGSAVTAGDVKSLR

SEQ ID NO: 224 - protein, 594 aa, Pseudomonas putida
MATVLNKVDAVIVGFGWAGAIMAKELTEAGLHVVALERGPMQDTYPEGSYPQVIDELTYSVRKKLFVDVSKETVTVRHSVNDVALPNRQLGAFLPGKGVGGAGLHWSGVHFRVDPVELRLRSHYEERYGRTFIPEGMTIQDFGVSYEELEPYFDFAEKVFGTSGQAWTVKGQVVGEGKGGNPYAPDRSNPFPLPAQKNVVSARLFEKAATSLGYKPYNLPSANTSGPYTNPYGAQMGPCNFCGFCSGYVCYMYSKASPNVNILPALRQVPNFELRPNAHVLRVNLDDSKRRATGVTYIDAQGREVEQPAELVILAAFQFNNVRLMLLSGIGKPYDPVKNEGVVGRNFAYQNMGTVKAFFDKDTYTNNFIGAGGNGIAIDDFNADNFDHGPHGFVGGSPMWVNQAGSRPIAGTSNPPGTPAWGSAWKKATADYYTHQVSMDAHGAHQSYRGNYLDLDPTYRDAYGQPLLRMTFDWQENDIKMNQFMVDKLSKIAQAMNPKAIAVLGKQVKDHFNTTSYQTTHLNGGAIMGTDPKTSALNRYLQSWDVHNVFVPGASAFPQGLGYNPTGLVAALTYWSARAIREQYLKNPGPLVQA

SEQ ID NO: 225 - protein, 246 aa, Pseudomonas putida
MPEHAPDNSRRDFLRKTLTLIPVVTVASTGLGAGASQLLAAPQQQPNVPATPPAGDYRPTFFSAEEWAFVQAAVSRIIPADELGPGALEAGAAEFIDRQMNTPYATGAQWYMQGPFNADAPPELGYQLQLSPQQIYRLGIAAVDGWCKANGGQVFAAQDSATRDRILSKLEAGELVFESVPAKVFFSLLVQNTREGFFCDPIHGGNKGMVGWTQIGFPGARADFMDWVERNEPYPFPAVSIRGERA

SEQ ID NO: 226 - protein, 527 aa, Burkholderia pseudomallei
MNKHWIRVSPIALAAAIGLTACGGDDVTSPGLSAVKNVVVIYAENRSFDNLYGNFPGANGLQNVTAASAAQVDRDGTPLATLPKIWGGLTAAGVTPAVTEAMTANLPNAPFAIDDPNGFNTPMSVTTRDLVHRFYENQMQIDGGKNDKYAAWSDAGGLVMGHYTADPSKLPLWKLAQQYTLADNFFMGTFGGSFLNHQYLICACAPFYPNADKSPAASQISVVNPDGVTLTTASNSPASALSGPPKFVKSGGLTPDFYAVNTMQPPYQPSANAAPTGGDPNLADPANPSTLPPQTQQNIGDLLTSAGVSWAWYAGAWGAALQAAQSGTSGVIYGPNMTSPNFQPHHQPFNYYANQAPGSANRAQHLLDAGSNGAALIQAIDAGKLPQVTFYKPQGNLNEHPGYTDVASGDQHIADVIAHLQKSPQWNNMVVIVTYDENGGFWDHVAPPKGDRWGPGTRIPAFVISPFAKQGFVDHTQYDTASILRFITRRFALPKLAGLKQRDDALVANGFKPMGDLTNALTLSTGN

SEQ ID NO: 227 - protein, 256 aa, Acetobacter pomorum
MRFRNAVFYAVSLSLAHTFFGHTAQAQQFTLPDGRAFLPPPPRAEEPAQQADLRAFEKTRGLKDKARWKLAQNDANLNPDHVIKDFSCAAGFNLDPAKLPAMVDLLTSLAQPVEQDVSNEKDFWKRRRPFVGTDKDICTAHSDGLDHSYAYPSGHTTWGWLTASILASALPDRATQIMQRGRIFGESRIVCGVHWKSDVQAGYMNGSAMFATLQEQPAFTQKMAKVRQELLALRNAKTEPDTKTCAVEQQAAQDIF

SEQ ID NO: 228 - protein, 236 aa, Morganella morganii
MRKLTLTLSALALALSLNSVADAKVYMPEKVSDGVTVAQLAEQHAIHWISVEQIEESLKGQPMAVGFDIDDTVLFSSPGFYRGKLEYSPNDYSYLKNPEFWEKMNNEWDKFSMPKKSGMELVQMHLKRGDTVYFITGRSKTKTETVTKYVQEGLRIPADKMNPVIFAGDEEGQNNKVSWMRDHKLKIYYGDADADIAAARELNIRGIRVLRASNSSYQPLPKAGQFGEEVVINSEY

SEQ ID NO: 229 - protein, 469 aa, Azospirillum brasilense
MSDISKVFDLIKEHDVKYVDLRFTDPRGKLHHTAQHVSTIDEDVFEDGIMFDGSSIAGWKAINESDMILQLDPTTAVMDPFSAQPTLNILCDVYEPSTGQPYARCPRGIAKAAEKYMASAGIGDTAYFGPEAEFFVFDDVKFKVEMNKVSYEFDSEEGPYTSDKDYEDGNLGHRPGVKGGYFPVAPVDSGSDLRAEMLSVLAEMGVPVEKHHHEVAASQHELGIKFDTLVRTGDNMQYYKYVVHNVAHAYGKTATFMPKPVFGDNGSGMHMHQSIWKEGQPLFAGNQYADLSELALYYIGGIIKHAKALNAFTNPTTNSYKRLVPGYEAPVLLAYSARNRSASCRIPYVASPKGKRVEVRFPDPSANPYLAFAALLMAGLDGIQNKIHPGEAMDKNLYDLPAEELAKVPTVCGSLREALDSLKADSAFLQKGDVFTKDMIESYIDLRTEELLAFETMPHPIEYKMYYSV

SEQ ID NO: 230 - protein, 468 aa, Pseudomonas stutzeri
MSKSLQLIKDYDVKWIDLRFTDTKGKQHHVTVPARDAQDEDFFEHGKMFDGSSIHGWKGIEASDMILMPVDETAVLDPFTEEPTLILVCDIVEPSTMQGYDRDPRSIAKRAEEFLKTTGIGDTVFVGPEPEFFIFDQVKFKSDISGSMFKIYSEQGSWMTDQDVEGGNKGHRPAVKGGYFPVPPCDHDHEIRTAMCNAMEEMGLVVEVHHHEVATAGQNEIGVKFNTLVAKADEVQTLKYCVHNVADAYGKTATFMPKPLYGDNGSGMHVHMSISKDGKNTFAGEGYAGLSETALYFIGGIIKHGKALNGFTNPSTNSYKRLVPGFEAPVMLAYSARNRSASIRIPYVSSPKARRIEARFPDPAANPYLCFAALLMAGLDGIQNKIHPGDAADKNLYDLPPEEGKLIPQVCGSLKEALEELDKGRAFLTKGGVFSDEFIDAYIELKSEEEIKVRTFVHPLEYDLYYSV

SEQ ID NO: 231 - protein, 1003 aa, Azospirillum brasilense
MTTTLPKPFDTALAGRGLERWRAEAATAADGTGDDDLGAWAEAYADSAEGRALIDAVCGNSPYLGQILTRELPFVREMVSEGYDAAFAALLRRLETDCAEERNIDRLMTALRIAKRRAALLIAMADITGAWELEAVTGALSDIAETSVRMAANHLLRRAAEAGTLTLPEPQRPWVGSGLIVLGMGKLGARELNYSSDIDLIVLYDDAVVQTPQPDNLARTFIRIARDLVRIMDERTKDGYVFRTDLRLRPDPGATPLAVSVSAAEIYYGSVGQNWERAAMIKARPIAGDPEAGAHFLRFLEPFVWRRHLDFAAIQDIHSIKRQINAHKGHREVTVNGHDIKVGRGGIREIEFFAQTQQLIFGGRDIRVRIAPTLLANKALCAVGRVPEAAVEELEEAYRFLRRVEHRIQMTDDRQTHQIPADDEGVAHLATFLGYERVEDFRADLLAQLGRVEDRYAELFEEAPSLSGPGNLVFTGTDDDPGTVKTLAGMGYRDPSRVIAVVSTWHRGRYRSTRSGRARELLTELVPAMLNELAKTPAPDDALVKFDSFLERLPAGVGLFSLFIANPWLLALVAEIMGTAPQLAETLSRNPSLLDAVLSPDFFDPLPDAAGLTPEYQRFIAGAHNFEDVLTLSRRWTNDQRFRAGAHILRGITDGDRCGPFLADLADVVVPELAARVEEEFAARHGRIPGGAWVVVAMGKLGSRQLTITSDIDLIVVYEVPPGTRQSDGAKPLAPNEYYIKLTQRLTNAITAPMADGRLYEVDMRLRPSGNAGPLATALDAFTAYQAKDAWTWEHMALTRARVIGSDSGGGDPALGHKVESAIRGVLTGPRDPAKVLRDVADMRRRIDKEFGTTNPWNVKYARGGLIDIEFTAQYLQLRHGHAHPDILSIATSRALLNAAAAGLLAPEVAEELVATLKLWRRVQGFLRLTTDGVLDPRQVSPTLREGLSRAAFPDEEPAVDFAALDSRIRDIAARAHRHFVALVEEPASRLPPPETNEEAKLP

SEQ ID NO: 232 - protein, 996 aa, Azorhizobium caulinodans
MPLRQAMTALTSPSPELGPGLAAAIREAPVLFDEAAAAVKLSHTLERFADHDRASLDALLVAHPMARQVVLGVMEGAPFLTDVIRREPARLLRALETDPVARVAALVAAAGVQVREAEDEAAVMRALRRMRSEAALVIALADIGGVLPLMDVTRALTRVADTAVALALDFLLREAAGSGKLLLPDLNVPGKGSGLAAIAMGKHGAGELNYSSDVDLILVYDRAVAPLGPDAEASPTYVRIAKGLVRLLQERTEDGYVLRVDLRLRPDPGSTQVALSTASALAYYEREGATWERAAYIKARPVAGDLVVGREYLDELAPFVWRRVLDYQAISDVHAMKREIHAFRGHEVVAVEGHNVKLGRGGIREVEFFVQTQQLIAGGRDRDLRSPRTLEALGALVDHRWIAPEVRDDLSEAYVFLRRVEHRLQMVADAQTHTLPEGREALEAFARFMGYKDRDSFAAALVERLQRVQGHYAHLFEDSVTHPCLQGELHFPLDRDDRATLGTLSRLGFRDPPAASRLVREWLAGQPRALRTDAAREHLARIVALLIEQLSHGGDPDGALNAMDSFLRDLPGPHLLAALEHNPDLVRLIATIVSAAPRLGETLARRPSLADALLDPAFFDVLPDEPALAAHLEALLDAAPNEEEQFDRARRFRQEQHVLIGVRIVSGTLPAARAGEAYARVAEVIIRALHARVWARFVEAHGSIPGADTAVLSMGKLGSREMTAGSDLDLIVLYDFDPASDGTSDGPRPLVGAQYFARFTQRLVTALTSLTNAGKLYDVDLRLRPSGRSGPVATRIGSFENYQRTEAWTWEHMALTRARIISASPAFARRVEQVILQVLAHPRDPRRIAGDILDMRRAIAAEKGESDRWNLKHAAGGQVDAEFLAQFLVLIHAERHPEIVDTATARILTVAGWLQLLSPEDCQTLSNACRLYQDLTQVLRLAIDRPFVPAQASPALKALLARAGEMPDFSSLDAHLTDTEARVRAIFERILEAASA

SEQ ID NO: 233 - protein, 946 aa, Escherichia coli
MKPLSSPLQQYWQTVVERLPEPLAEESLSAQAKSVLTFSDFVQDSISAHPEWLTELESQPPQADEWQHYAAWLQEALCNVSDEAGLMRELRLFRRRIMVRIAWAQTLALVTEESILQQLSYLAETLIVAARDWLYDACCREWGTPCNAQGEAQPLLILGMGKLGGGELNFSSDIDLIFAWPEHGCTQGGRRELDNAQFFTRMGQRLIKVLDQPTQDGFVYRVDMRLRPFGESGPLVLSFAALEDYYQEQGRDWERYAMVKARIMGDSDGVYANELRAMLRPFVFRRYIDFSVIQSLRNMKGMIAREVRRRGLTDNIKLGAGGIREIEFIVQVFQLIRGGREPSLQSRSLLPTLSAIAELHLLSENDAEQLRVAYLFLRRLENLLQSINDEQTQTLPSDELNRARLAWAMDFADWPQLTGALTAHMTNVRRVFNELIGDDESETQEESLSEQWRELWQDALQEDDTTPVLAHLSEDDRKQVLTLIADFRKELDKRTIGPRGRQVLDHLMPHLLSDVCAREDAAVTLSRITALLVGIVTRTTYLELLSEFPAALKHLISLCAASPMIASQLARYPLLLDELLDPNTLYQPTATDAYRDELRQYLLRVPEDDEEQQLEALRQFKQAQLLRIAAADIAGTLPVMKVSDHLTWLAEAMIDAVVQQAWVQMVARYGKPNHLNEREGRGFAVVGYGKLGGWELGYSSDLDLIFLHDCPMDAMTDGEREIDGRQFYLRLAQRIMHLFSTRTSSGILYEVDARLRPSGAAGMLVTSAEAFADYQKNEAWTWEHQALVRARVVYGDPQLTAHFDAVRREIMTLPREGKTLQTEVREMREKMRAHLGNKHRDRFDIKADEGGITDIEFITQYLVLRYAHEKPKLTRWSDNVRILELLAQNDIMEEQEAMALTRAYTTLRDELHHLALQELPGHVSEDCFTAERELVRASWQKWLVEE

SEQ ID NO: 234 - DNA, 18 bp, Escherichia coli
  source 1..18: mol_type = genomic DNA
gttggccttctggccgcc

SEQ ID NO: 235 - DNA, 18 bp, Escherichia coli
  source 1..18: mol_type = genomic DNA
gttgccggtttgttcatg

SEQ ID NO: 236 - DNA, 18 bp, Escherichia coli
  source 1..18: mol_type = genomic DNA
gttgaccttctccttacc

SEQ ID NO: 237 - DNA, 18 bp, Escherichia coli
  source 1..18: mol_type = genomic DNA
gttgaccttctccttacc

SEQ ID NO: 238 - DNA, 18 bp, Escherichia coli
  source 1..18: mol_type = genomic DNA
gttgaccttctccttacc

SEQ ID NO: 239 - DNA, 18 bp, Escherichia coli
  source 1..18: mol_type = genomic DNA
gttgaccgtgcgttcctg

SEQ ID NO: 240 - DNA, 18 bp, Escherichia coli
  source 1..18: mol_type = genomic DNA
gttgaccgggcgttcacg

SEQ ID NO: 241 - DNA, 18 bp, Escherichia coli
  source 1..18: mol_type = genomic DNA
gttctcgccctggttacc

SEQ ID NO: 242 - DNA, 18 bp, Escherichia coli
  source 1..18: mol_type = genomic DNA
gttgaactggtctacacc

SEQ ID NO: 243 - DNA, 18 bp, Escherichia coli
  source 1..18: mol_type = genomic DNA
gttctcgccctggacacg

SEQ ID NO: 244 - DNA, 18 bp, Escherichia coli
  source 1..18: mol_type = genomic DNA
gttctccggctgttccgc

SEQ ID NO: 245 - DNA, 18 bp, Escherichia coli
  source 1..18: mol_type = genomic DNA
gttcgtgctctgttcgtg

SEQ ID NO: 246 - DNA, 18 bp, Escherichia coli
  source 1..18: mol_type = genomic DNA
gttcggctgctggccgcc

SEQ ID NO: 247 - DNA, 18 bp, Escherichia coli
  source 1..18: mol_type = genomic DNA
gttcggctgctggccgcc

SEQ ID NO: 248 - DNA, 18 bp, Escherichia coli
  source 1..18: mol_type = genomic DNA
gttcgcctccgtttcact

SEQ ID NO: 249 - DNA, 18 bp, Escherichia coli
  source 1..18: mol_type = genomic DNA
gttcgctttgcggacacc

SEQ ID NO: 250 - DNA, 18 bp, Escherichia coli
  source 1..18: mol_type = genomic DNA
gttcgcctacggtacgtg

SEQ ID NO: 251 - DNA, 18 bp, Escherichia coli
  source 1..18: mol_type = genomic DNA
gttcgcctccgtttcact

SEQ ID NO: 252 - DNA, 18 bp, Escherichia coli
  source 1..18: mol_type = genomic DNA
gttccgcttcttttcagc

SEQ ID NO: 253 - DNA, 18 bp, Escherichia coli
  source 1..18: mol_type = genomic DNA
gttcctcctccattcact

SEQ ID NO: 254 - DNA, 18 bp, Escherichia coli
  source 1..18: mol_type = genomic DNA
gttcccgtgctgctcatt

SEQ ID NO: 255 - DNA, 18 bp, Escherichia coli
  source 1..18: mol_type = genomic DNA
gttcatgtgctgttcacg

SEQ ID NO: 256 - DNA, 18 bp, Escherichia coli
  source 1..18: mol_type = genomic DNA
gttcatcttccgttcacc

SEQ ID NO: 257 - DNA, 18 bp, Escherichia coli
  source 1..18: mol_type = genomic DNA
gttcatgtctacttcatc

SEQ ID NO: 258 - DNA, 18 bp, Escherichia coli
  source 1..18: mol_type = genomic DNA
gttcatcttccgttcacc

SEQ ID NO: 259 - DNA, 18 bp, Escherichia coli
  source 1..18: mol_type = genomic DNA
gttcatctcctgggcgcg

SEQ ID NO: 260 - DNA, 18 bp, Escherichia coli
  source 1..18: mol_type = genomic DNA
gttcagctcccagccgcc

SEQ ID NO: 261 - DNA, 18 bp, Escherichia coli
  source 1..18: mol_type = genomic DNA
gttcagcgttcgtttatc

SEQ ID NO: 262 - DNA, 18 bp, Escherichia coli
  source 1..18: mol_type = genomic DNA
gttcagatggcggtcgtg

SEQ ID NO: 263 - DNA, 18 bp, Escherichia coli
  source 1..18: mol_type = genomic DNA
gttcacgttctcgctgtc

SEQ ID NO: 264 - DNA, 18 bp, Escherichia coli
  source 1..18: mol_type = genomic DNA
gttcacgtgctggacacc

SEQ ID NO: 265 - DNA, 18 bp, Escherichia coli
  source 1..18: mol_type = genomic DNA
gttcacgttcgataaacg

SEQ ID NO: 266 - DNA, 18 bp, Escherichia coli
  source 1..18: mol_type = genomic DNA
gttcacgtgccgctcgtt

SEQ ID NO: 267 - DNA, 18 bp, Escherichia coli
  source 1..18: mol_type = genomic DNA
gttcacggcgtggttggc

SEQ ID NO: 268 - DNA, 18 bp, Escherichia coli
  source 1..18: mol_type = genomic DNA
gttcaccttggagccagg

SEQ ID NO: 269 - DNA, 18 bp, Escherichia coli
  source 1..18: mol_type = genomic DNA
gttcaccttggagccagg

SEQ ID NO: 270 - DNA, 18 bp, Escherichia coli
  source 1..18: mol_type = genomic DNA
gttcacctcggagtggtc

SEQ ID NO: 271 - DNA, 18 bp, Escherichia coli
  source 1..18: mol_type = genomic DNA
gttcaccttctggccttc

SEQ ID NO: 272 - DNA, 18 bp, Escherichia coli
  source 1..18: mol_type = genomic DNA
gttcaccgggagttcggc

SEQ ID NO: 273 - DNA, 18 bp, Escherichia coli
  source 1..18: mol_type = genomic DNA
gttcaccgggagttcggc

SEQ ID NO: 274 - DNA, 18 bp, Escherichia coli
  source 1..18: mol_type = genomic DNA
gttcacctacgggcaacg

SEQ ID NO: 275 - DNA, 18 bp, Escherichia coli
  source 1..18: mol_type = genomic DNA
gttcaccgcgtgtcaagg

SEQ ID NO: 276 - DNA, 18 bp, Escherichia coli
  source 1..18: mol_type = genomic DNA
gttcaccgcgtgtcaagg

SEQ ID NO: 277 - DNA, 18 bp, Escherichia coli
  source 1..18: mol_type = genomic DNA
gttcaccctgtgtgcact

SEQ ID NO: 278 - DNA, 18 bp, Escherichia coli
  source 1..18: mol_type = genomic DNA
gttcaccctctccccacc

SEQ ID NO: 279 - DNA, 18 bp, Escherichia coli
  source 1..18: mol_type = genomic DNA
gttcacccggcgttcatt

SEQ ID NO: 280 - DNA, 18 bp, Escherichia coli
  source 1..18: mol_type = genomic DNA
gttcaccctctccccacc

SEQ ID NO: 281 - DNA, 18 bp, Escherichia coli
  source 1..18: mol_type = genomic DNA
gttcaccaggcgttcctg

SEQ ID NO: 282 - DNA, 18 bp, Escherichia coli
  source 1..18: mol_type = genomic DNA
gttcacatcctgttagcc

SEQ ID NO: 283 - DNA, 18 bp, Escherichia coli
  source 1..18: mol_type = genomic DNA
gttcaactgccgttcacc

SEQ ID NO: 284 - DNA, 18 bp, Escherichia coli
  source 1..18: mol_type = genomic DNA
gttcaacttgcgttcagg

SEQ ID NO: 285 - DNA, 18 bp, Escherichia coli
  source 1..18: mol_type = genomic DNA
gttcaactccggcccagg

SEQ ID NO: 286 - DNA, 18 bp, Escherichia coli
  source 1..18: mol_type = genomic DNA
gttcaactgccgttcacc

SEQ ID NO: 287 - DNA, 18 bp, Escherichia coli
  source 1..18: mol_type = genomic DNA
gtccatcccgcggtaatc

SEQ ID NO: 288 - DNA, 18 bp, Escherichia coli
  source 1..18: mol_type = genomic DNA
gttaaccggctgttgacc

SEQ ID NO: 289 - DNA, 18 bp, Escherichia coli
  source 1..18: mol_type = genomic DNA
gtccacgtgtgcgtcatg

SEQ ID NO: 290 - DNA, 18 bp, Escherichia coli
  source 1..18: mol_type = genomic DNA
gtccaccgtctggtcctc

SEQ ID NO: 291 - DNA, 18 bp, Escherichia coli
  source 1..18: mol_type = genomic DNA
gtcaacgtcttgttaacg

SEQ ID NO: 292 - DNA, 18 bp, Escherichia coli
  source 1..18: mol_type = genomic DNA
gtcatcgtcgtgttcatg

SEQ ID NO: 293 - DNA, 18 bp, Escherichia coli
  source 1..18: mol_type = genomic DNA
ggtgacccgccggtcaca

SEQ ID NO: 294 - DNA, 18 bp, Escherichia coli
  source 1..18: mol_type = genomic DNA
ggtgacccagcggtcgtc

SEQ ID NO: 295 - DNA, 18 bp, Escherichia coli
  source 1..18: mol_type = genomic DNA
ggtcgcccgccgttcgcc

SEQ ID NO: 296 - DNA, 18 bp, Escherichia coli
  source 1..18: mol_type = genomic DNA
ggtctcccgctcgtcatg

SEQ ID NO: 297 - DNA, 18 bp, Escherichia coli
  source 1..18: mol_type = genomic DNA
ggtcgcccgccgttcgcc

SEQ ID NO: 298 - DNA, 18 bp, Escherichia coli
  source 1..18: mol_type = genomic DNA
ggtccacttccgtccagc

SEQ ID NO: 299 - DNA, 18 bp, Escherichia coli
  source 1..18: mol_type = genomic DNA
ggtcagcggtcggtcgtg

SEQ ID NO: 300 - DNA, 18 bp, Escherichia coli
  source 1..18: mol_type = genomic DNA
ggtcatcgccccgccact

SEQ ID NO: 301 - DNA, 18 bp, Escherichia coli
  source 1..18: mol_type = genomic DNA
ggtcacgtttcgatcacc

SEQ ID NO: 302 - DNA, 18 bp, Escherichia coli
  source 1..18: mol_type = genomic DNA
ggtcacgcccgagaaatc

SEQ ID NO: 303 - DNA, 18 bp, Escherichia coli
  source 1..18: mol_type = genomic DNA
ggtcaccgtgcctccaca

SEQ ID NO: 304 - DNA, 18 bp, Escherichia coli
  source 1..18: mol_type = genomic DNA
ggtcacgcccgagaaatc

SEQ ID NO: 305 - DNA, 18 bp, Escherichia coli
  source 1..18: mol_type = genomic DNA
ggtcacccgggagccacc

SEQ ID NO: 306 - DNA, 18 bp, Escherichia coli
  source 1..18: mol_type = genomic DNA
ggtcaccgcggggccagc

SEQ ID NO: 307 - DNA, 18 bp, Escherichia coli
  source 1..18: mol_type = genomic DNA
ggtcacccggagctcacc

SEQ ID NO: 308 - DNA, 18 bp, Escherichia coli
  source 1..18: mol_type = genomic DNA
ggtcacccggagctcacc

SEQ ID NO: 309 - DNA, 18 bp, Escherichia coli
  source 1..18: mol_type = genomic DNA
ggtcacattttcgtcaca

SEQ ID NO: 310 - DNA, 18 bp, Escherichia coli
  source 1..18: mol_type = genomic DNA
ggtcaccccctcgacggc

SEQ ID NO: 311 - DNA, 18 bp, Escherichia coli
  source 1..18: mol_type = genomic DNA
ggtcaactgcccgtcgcc

SEQ ID NO: 312 - DNA, 18 bp, Escherichia coli
  source 1..18: mol_type = genomic DNA
ggtcacacggccttcaca

SEQ ID NO: 313 - DNA, 18 bp, Escherichia coli
  source 1..18: mol_type = genomic DNA
ggtcaaccgccggtcgct

SEQ ID NO: 314 - DNA, 18 bp, Escherichia coli
  source 1..18: mol_type = genomic DNA
ggtcaactgcccgtcgcc

SEQ ID NO: 315 - DNA, 18 bp, Escherichia coli
  source 1..18: mol_type = genomic DNA
ggtatcccgcccgtcacc

SEQ ID NO: 316 - DNA, 18 bp, Escherichia coli
  source 1..18: mol_type = genomic DNA
ggtatcccgcccgtcacc

SEQ ID NO: 317 - DNA, 18 bp, Escherichia coli
  source 1..18: mol_type = genomic DNA
ggtcaaccgccggtaacc

SEQ ID NO: 318 - DNA, 18 bp, Escherichia coli
  source 1..18: mol_type = genomic DNA
ggtaacacggggttcacc

SEQ ID NO: 319 - DNA, 18 bp, Escherichia coli
  source 1..18: mol_type = genomic DNA
ggtaaccccggggcaacc

SEQ ID NO: 320 - DNA, 18 bp, Escherichia coli
  source 1..18: mol_type = genomic DNA
ggccaccccgcggtcacg

SEQ ID NO: 321 - DNA, 18 bp, Escherichia coli
  source 1..18: mol_type = genomic DNA
ggccacgtcctggccacc

SEQ ID NO: 322 - DNA, 18 bp, Escherichia coli
  source 1..18: mol_type = genomic DNA
gatcacccgtcggtaagc

SEQ ID NO: 323 - DNA, 18 bp, Escherichia coli
  source 1..18: mol_type = genomic DNA
ggccacatccccttcacc

SEQ ID NO: 324 - DNA, 18 bp, Escherichia coli
  source 1..18: mol_type = genomic DNA
gatcaccctgcgtctatg

SEQ ID NO: 325 - DNA, 18 bp, Escherichia coli
  source 1..18: mol_type = genomic DNA
gatcacaccttcttcacg

SEQ ID NO: 326 - DNA, 18 bp, Escherichia coli
  source 1..18: mol_type = genomic DNA
gatcacccgtcggtaagc

SEQ ID NO: 327 - DNA, 18 bp, Escherichia coli
  source 1..18: mol_type = genomic DNA
cttcgcctccccttcatc

SEQ ID NO: 328 - DNA, 18 bp, Escherichia coli
  source 1..18: mol_type = genomic DNA
cttcgcctccccttcatc

SEQ ID NO: 329 - DNA, 18 bp, Escherichia coli
  source 1..18: mol_type = genomic DNA
cttcaaccacggtcaacc

SEQ ID NO: 330 - DNA, 18 bp, Escherichia coli
  source 1..18: mol_type = genomic DNA
cttcgcctccccttcatc

SEQ ID NO: 331 - DNA, 18 bp, Escherichia coli
  source 1..18: mol_type = genomic DNA
cgtcacttcgtgttcacg

SEQ ID NO: 332 - DNA, 18 bp, Escherichia coli
  source 1..18: mol_type = genomic DNA
cgtcacttcgtgttcacg

SEQ ID NO: 333 - DNA, 18 bp, Escherichia coli
  source 1..18: mol_type = genomic DNA
cgtcacgtcgcggtcacc

SEQ ID NO: 334 - DNA, 18 bp, Escherichia coli
  source 1..18: mol_type = genomic DNA
attcgtccattgttcacc

SEQ ID NO: 335 - DNA, 18 bp, Escherichia coli
  source 1..18: mol_type = genomic DNA
attcgggtggtgttcatc

SEQ ID NO: 336 - DNA, 18 bp, Escherichia coli
  source 1..18: mol_type = genomic DNA
attcgtccattgttcacc

SEQ ID NO: 337 - DNA, 18 bp, Escherichia coli
  source 1..18: mol_type = genomic DNA
attcccctggcgttcgcc

SEQ ID NO: 338 - DNA, 18 bp, Escherichia coli
  source 1..18: mol_type = genomic DNA
attcgggtggtgttcatc

SEQ ID NO: 339 - DNA, 18 bp, Escherichia coli
  source 1..18: mol_type = genomic DNA
attcagcctgtggccact

SEQ ID NO: 340 - DNA, 18 bp, Escherichia coli
  source 1..18: mol_type = genomic DNA
attcagcctgtggccact

SEQ ID NO: 341 - DNA, 18 bp, Escherichia coli
  source 1..18: mol_type = genomic DNA
attcactcaccgttcagt

SEQ ID NO: 342 - DNA, 18 bp, Escherichia coli
  source 1..18: mol_type = genomic DNA
attcactcaccgttcagt

SEQ ID NO: 343 - DNA, 18 bp, Escherichia coli
  source 1..18: mol_type = genomic DNA
attcacgcgccggccacc

SEQ ID NO: 344 - DNA, 18 bp, Escherichia coli
  source 1..18: mol_type = genomic DNA
agtaaaccctcgttcact

SEQ ID NO: 345 - DNA, 18 bp, Escherichia coli
  source 1..18: mol_type = genomic DNA
agtaaaccctcgttcact

SEQ ID NO: 346 - DNA, 18 bp, Streptomyces coelicolor
  source 1..18: mol_type = genomic DNA
attaaccaaatcgtcaca

SEQ ID NO: 347 - DNA, 18 bp, Streptomyces coelicolor
  source 1..18: mol_type = genomic DNA
tgtcattaaaaagaaaca

SEQ ID NO: 348 - DNA, 18 bp, Streptomyces coelicolor
  source 1..18: mol_type = genomic DNA
tgtaatgtaaccgtcaat

SEQ ID NO: 349 - DNA, 18 bp, Streptomyces coelicolor
  source 1..18: mol_type = genomic DNA
tttcacgccgttgtaata

SEQ ID NO: 350 - DNA, 18 bp, Streptomyces coelicolor
  source 1..18: mol_type = genomic DNA
tgtaatagttctgtaacg

SEQ ID NO: 351 - DNA, 18 bp, Streptomyces coelicolor
  source 1..18: mol_type = genomic DNA
tgtcataaaactgtcata

SEQ ID NO: 352 - DNA, 18 bp, Streptomyces coelicolor
  source 1..18: mol_type = genomic DNA
ttacatacaaatgtaata

SEQ ID NO: 353 - DNA, 18 bp, Streptomyces coelicolor
  source 1..18: mol_type = genomic DNA
ggtcaccgagttgtcata

SEQ ID NO: 354 - DNA, 18 bp, Streptomyces coelicolor
  source 1..18: mol_type = genomic DNA
tttcacaatgttgtcatg

SEQ ID NO: 355 - DNA, 18 bp, Streptomyces coelicolor
  source 1..18: mol_type = genomic DNA
ggtaataaatatgtcact

SEQ ID NO: 356 - DNA, 18 bp, Streptomyces coelicolor
  source 1..18: mol_type = genomic DNA
tgtcatctttctgacacc

SEQ ID NO: 357 - DNA, 18 bp, Streptomyces coelicolor
  source 1..18: mol_type = genomic DNA
cttcacagtaccgtcatc

SEQ ID NO: 358 - DNA, 18 bp, Streptomyces coelicolor
  source 1..18: mol_type = genomic DNA
tgtcatacacccgtcacg

SEQ ID NO: 359 - DNA, 18 bp, Streptomyces coelicolor
  source 1..18: mol_type = genomic DNA
tgacacaaatctttaatc

SEQ ID NO: 360 - DNA, 18 bp, Streptomyces coelicolor
  source 1..18: mol_type = genomic DNA
agtaacaaccgtgtcaca

SEQ ID NO: 361 - DNA, 18 bp, Streptomyces coelicolor
  source 1..18: mol_type = genomic DNA
tttcacatttctgtgaca

SEQ ID NO: 362 - DNA, 18 bp, Streptomyces coelicolor
  source 1..18: mol_type = genomic DNA
tgtcacaattcgatcatg

SEQ ID NO: 363 - DNA, 18 bp, Streptomyces coelicolor
  source 1..18: mol_type = genomic DNA
tgtcatcaaactgccatt

SEQ ID NO: 364 - DNA, 18 bp, Streptomyces coelicolor
  source 1..18: mol_type = genomic DNA
tttcacagagcgaaaacg

SEQ ID NO: 365 - DNA, 18 bp, Streptomyces coelicolor
  source 1..18: mol_type = genomic DNA
tatcagaaaaatgtcatg

SEQ ID NO: 366 - DNA, 18 bp, Streptomyces coelicolor
  source 1..18: mol_type = genomic DNA
agtcatatttctgtcaca

SEQ ID NO: 367 - DNA, 18 bp, Streptomyces coelicolor
  source 1..18: mol_type = genomic DNA
tgtcatcactctgtcatc

SEQ ID NO: 368 - DNA, 18 bp, Streptomyces coelicolor
  source 1..18: mol_type = genomic DNA
tgtcacattcctgtcaat

SEQ ID NO: 369 - DNA, 18 bp, Streptomyces coelicolor
  source 1..18: mol_type = genomic DNA
tgtcaggccgctgtcatc

SEQ ID NO: 370 - DNA, 18 bp, Streptomyces coelicolor
  source 1..18: mol_type = genomic DNA
tgtcatttttctgtcacc

SEQ ID NO: 371 - DNA, 18 bp, Streptomyces coelicolor
  source 1..18: mol_type = genomic DNA
attcacagcactgtcata

SEQ ID NO: 372 - DNA, 18 bp, Streptomyces coelicolor
  source 1..18: mol_type = genomic DNA
tttcataaatctgtcata

SEQ ID NO: 373 - DNA, 18 bp, Streptomyces coelicolor
  source 1..18: mol_type = genomic DNA
tgtaacagaaatatcaca

SEQ ID NO: 374 - DNA, 18 bp, Streptomyces coelicolor
  source 1..18: mol_type = genomic DNA
tgtcataaagttgtcacg

SEQ ID NO: 375 - DNA, 18 bp, Streptomyces coelicolor
  source 1..18: mol_type = genomic DNA
tgtaataaaagcgtaaac

SEQ ID NO: 376 - DNA, 18 bp, Azorhizobium caulinodans
  source 1..18: mol_type = genomic DNA
gtgtcacgaaccagtcat

SEQ ID NO: 377 - DNA, 18 bp, Azospirillum brasilense
  source 1..18: mol_type = genomic DNA
tcgtcatgaaactgtcat

SEQ ID NO: 378 - DNA, 18 bp, Escherichia coli
  source 1..18: mol_type = genomic DNA
ctgtcataaagttgtcac

SEQ ID NO: 379 - DNA, 18 bp, Escherichia coli
  source 1..18: mol_type = genomic DNA
tcatcacatcgttgtcat

SEQ ID NO: 380 - DNA, 18 bp, Escherichia coli
  source 1..18: mol_type = genomic DNA
atgacaaaaagatgacat

SEQ ID NO: 381 - DNA, 18 bp, Escherichia coli
  source 1..18: mol_type = genomic DNA
ctgtcataaaaatgtcat

SEQ ID NO: 382 - DNA, 18 bp, Escherichia coli
  source 1..18: mol_type = genomic DNA
ctgtcacctgtttgtcct

SEQ ID NO: 383 - DNA, 18 bp, Gluconacetobacter diazotrophicus
  source 1..18: mol_type = genomic DNA
ttcggccacatgtgtcgt

SEQ ID NO: 384 - DNA, 18 bp, Gluconacetobacter diazotrophicus
  source 1..18: mol_type = genomic DNA
ttgccgaatggctgccat

SEQ ID NO: 385 - DNA, 18 bp, Herbaspirillum seropedicae
  source 1..18: mol_type = genomic DNA
tgatcaaatctgttgcag

SEQ ID NO: 386 - DNA, 18 bp, Kosakonia radicincitans
  source 1..18: mol_type = genomic DNA
attattgatatgccgcac

SEQ ID NO: 387 - DNA, 18 bp, Kosakonia radicincitans
  source 1..18: mol_type = genomic DNA
ttatcacgttgcgctgat

SEQ ID NO: 388 - DNA, 18 bp, Kosakonia radicincitans
  source 1..18: mol_type = genomic DNA
ttgtgaattctcattcgt

SEQ ID NO: 389 - DNA, 18 bp, Kosakonia radicincitans
  source 1..18: mol_type = genomic DNA
ttatcttttttatatcat

SEQ ID NO: 390 - DNA, 18 bp, Kosakonia radicincitans
  source 1..18: mol_type = genomic DNA
tattcaaaaatctatcaa

SEQ ID NO: 391 - DNA, 18 bp, Paenibacillus azotofixans
  source 1..18: mol_type = genomic DNA
tagacggatttgcggcga

SEQ ID NO: 392 - DNA, 18 bp, Pseudomonas stutzeri
  source 1..18: mol_type = genomic DNA
tggctggttctgacatcg

SEQ ID NO: 393 - DNA, 18 bp, Pseudomonas stutzeri
  source 1..18: mol_type = genomic DNA
gcgaagctgctctgaagg

SEQ ID NO: 394 - DNA, 18 bp, Pseudomonas stutzeri
  source 1..18: mol_type = genomic DNA
ggggcttttttttgcgac

SEQ ID NO: 395 - DNA, 18 bp, Pseudomonas stutzeri
  source 1..18: mol_type = genomic DNA
gcgtcatggctccgacaa

SEQ ID NO: 396 - DNA, 18 bp, Pseudomonas stutzeri
  source 1..18: mol_type = genomic DNA
ctgtcacagaaccgccac

SEQ ID NO: 397 - DNA, 18 bp, Pseudomonas stutzeri
  source 1..18: mol_type = genomic DNA
ttgtcacacttgcttcat

SEQ ID NO: 398 - protein, 471 aa, Kosakonia radicincitans
MKQSALYLALLPVFFTSVIYAETTDTSVLEHRAAQGDISTPGGARRLTADQTAALLDSLIDKPAKNIILLIGDGMGDSEITAARNYAEGAGGYFKGIDALPLTGQYTHYALDKKTGKPDYVTDSAASATAWSTGVKTYNGALGVDIHEKDHMTILEMAKAAGLATGNVSTAELQDATPAALVSHVISRRCYGPTVTSEKCALNALEKGGKGSITEQFLNTRADVTLGGGAKTFSETATAGKWQGKTLREQAQARGYQMVSDASALAAISEANQDKPLLGLFADGNMPVRWEGPKASLHGNLDKPAVTCTPNAKRTDSVPTLAAMTEKAIDLLSKNEKGFFLQVEGASIDKQDHAANPCGQIGETVDLDEAVQKALEFAKKDGNTLVIVTADHAHASQIIPADTKAPGLSQALNTKDGAVMAITYGNSEEESMEHTGTQVRIAAYGPHAGNVVGLTDQTDLFYTMKSALNLK

SEQ ID NO: 399 - protein, 346 aa, Kosakonia radicincitans
MNVMRTTVATVVAATFSLSAFAVNAAASLTGAGATFPAPVYAKWADTYQKETGNKINYQGIGSSGGVKQITAKTVDFGASDAPLADDKLAQEGLFQFPTVIGGIVLAINVPGIKSGELVLDGKTLGDIYLGNIKKWDDAAIAKLNPGLKLPSQNIAVVRRADGSGTSFVFTNYLAKVNEEWKSKVGSGSTVNWPTGLGGKGNDGIAAFVQRLPGSIGYVEYAYAKQNNLTYTKLLSADGKAVSPTEQSFSNAAKNIDWSKSFAQDLTNQKGADVWPITSTTFILVHKEQAKPEQGAEVLKFFDWAYKKGGKQANDLDYATLPDSVVEQVRAAWKTNVKDSSGKALY

SEQ ID NO: 400 - protein, 573 aa, Paenibacillus durus
MNKGLKKVAIGGLACLILSAGSFAYAAQSASKTQKAVKAPSKNLIVLIGDGMGPAQISATRYYEQYKKGVKHLNLDPYYVGQATTYADRGEDGGKVVSGVVTDSASAGTAFATGHKTYNAGISVSNEDASKPFASVIEAAVSSGKATGLVTTARITHATPAVYASHVRNRDNEAAIASQYLESGVDVLFGGGKQFFVTKDEKGKRTDKNILPDFKAKGYTIVESTSALKALPASTTKALGLFGNSHVAYTPDRTAEIPSLAAMTSSALKILSKDKDGFVMMIEGGRIDHAGHANDFPTLIQETLDFDAAFKTAIEFAKKDGNTSVVVTADHETGGLSLSRDNIYELNIDLWDKQKNSSESIASKLAEAKTPEEIRSIVAENTGINDLTDEEVQAILAGDGSSYKQEGAYNAVISKRLLVGWSGHGHSAVDVGIWAYGPIAGKVKGQVDNTQIARASAAIVGVNLDKRSAELQSKYVYPKFKINRENEVLYPVASLIKALGGSYSSGSSVSKAAFGKNTLNINMSALTATLNGKAVAFTVDNDNGTVYLPLSAFNQLTGKNLKWDSLSERIVLR

SEQ ID NO: 401 - protein, 316 aa, Paenibacillus durus
MRSKKSWIMALALTSVLALSACGNNGGNNTAANEGNTGASSAPTETSGAEVSGSILASGSTALQPLVEQVAEKFMEKNAGVDIQVQGGGSGTGLTQVAEKQVDIGNSDVFAEEKLKDADAEKAKALVDHQVAVVAIAAVSHPAAGVDTLTKQQLVDIFTGKITNWKEVGGADQKIQIINRPASSGTRATFEKFALGTKTEDLPGSIQEDSSGTVKKIIGETPGAIGYLALSYLDDSVKTLNYDGVEPSVDNVVSGKYPVWAYEHMYTNGEPNATVKAFLDYFQTDEVQNGDVTELGYIPASKMQVSRDVAGNVTNK

SEQ ID NO: 402 - protein, 580 aa, Azorhizobium caulinodans
MRRLLAALALSTALAGTAGATAIYPLDRATILAGSPFDLKVEFDKEVQPQDVSITINGQPVEQVLGKKPDFIAKEEGVNASAVLLRAAAFPKPGTYKVEAKAGGETKTVTWDVYATAATPKAKNVIFFLGDGLSVAHRTGARLMSKGMTEGKANGRLNIDDFERMAFIGTSSTNAIATDSANTMSAYMTGHKTAVNALGVYADRTKSTLDDPKVETIAEAIRRTSKKSVGIVTGAEVEDATPAAVVAHTRARADKADIVGMMLDVKPDVVLGGGSAYFLPKTTPGSKRKDDKDYIQLFKDAGYTLATDKTELASAAGTNTGKILGLFHPANMDVTLDREFLKKGTVEKFPNQPGLVEMTKVALNQLAKNPDGFFLMVEGASIDKMSHPMDWERALFETIEFDQAVGVGIEFLKTHPDTLIVVTGDHTHGISIIGTIDDNKPGPDMRDKVGVYADAGFPNYEDKNGDGFPDKVDVSRRLAVFASNYPDYYETWRPKMDGPFDPAVQNEKKQYVANEAYKNVPGATFREGNLPRSADTAVHAVDDVVLQATGAGADGFKGYMEESDVYRVLVDVMALAPAKN

SEQ ID NO: 403 - protein, 347 aa, Azorhizobium caulinodans
MKLTHILGAALAVAGTLVAGNASALDFNGAGASFPAPVYQSWGAKYKEKTGNGLNYQSIGSGGGQNQIINRTVDFGASDAPVEGPKLASNNLLQFPAVIGSIVATVNLDGIQTEQLKLTGPVLADIYLGKITKWNDKAIADLNAGVKLPDQAIVPVYRSDASGTSYVFTSYLAEVSADWKAKVGAATSVQWPTGAGAKGNEGVAGTVKNTKGAIGYVEFVYAAANKLVVTQLQNKAGKFVAPTVPAFMEAAAAADWAGSKDFAASMLNTPGANAWPIVSATYILLPKNPTNVDASKEVMKFFQWAYTKEGGEIAEKLHYIPLPEAVVKRVEEAWKAEVKAPDGKPVF

SEQ ID NO: 404 - protein, 471 aa, Escherichia coli
MKQSTIALALLPLLFTPVTKARTPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 405 - protein, 346 aa, Escherichia coli
MKVMRTTVATVVAATLSMSAFSVFAEASLTGAGATFPAPVYAKWADTYQKETGNKVNYQGIGSSGGVKQIIANTVDFGASDAPLSDEKLAQEGLFQFPTVIGGVVLAVNIPGLKSGELVLDGKTLGDIYLGKIKKWDDEAIAKLNPGLKLPSQNIAVVRRADGSGTSFVFTSYLAKVNEEWKNNVGTGSTVKWPIGLGGKGNDGIAAFVQRLPGAIGYVEYAYAKQNNLAYTKLISADGKPVSPTEENFANAAKGADWSKTFAQDLTNQKGEDAWPITSTTFILIHKDQKKPEQGTEVLKFFDWAYKTGAKQANDLDYASLPDSVVEQVRAAWKTNIKDSSGKPLY

SEQ ID NO: 406 - protein, 524 aa, Azoarcus olearius
MQPKALAISIALALCAAQAHAAGNAVPTSAEEWFAAGRAAVETSKHVVPNRARAKNVILFVGDGMGISTVTAARILEGQMRNADGEFNRLSFEKLDHMGTSVTASANQQTSDSAPTATAMVTGIKTNDGAISVDQTIERNEPSATVTRAKSVKTILEQAEERGMSTGIVTTARLTHATPAVNYAHIGNRDWEADSNLPAGATVADIARQLLEFPYGDGLEVALGGGRSYFMPNTASDPEYPSQKGRRKDGRDLTKEWTTKYKQSAYVWDKAAFDAVNPQRTKHLLGLFERSHMRYEADRKDDVAGEPSLAEMTEKAIKMLGQNKKGFYLMVEAGRIDHAHHAGNAYRALTDTVALSEAVEVAKRLTDDQDTLIVVTADHSHVFTIAGYPSRGNPILGKSAIDGVASTDALGLTYTTVSYANGPGWTGGFQRKEYNPATEGTVAAPYNGSALRPNLAGIDTTAPNYMQEATVPMGSETHAGEDVAIYASGPNAYLFRGPQEQNVIYHVMADALGLNKGGHGRGRD

SEQ ID NO: 407 - protein, 347 aa, Azoarcus olearius
MAIRFLLLLSLAWSACVAAAPDVLRGAGSSAAQPVYAAWAAAYAATHGTVLEYDPAGSGAGIKKLLAAEVDFGASDLVPDAAALQGRDIVVVPTAVTGAVPVINLPGLRGALRLDGATLAEIFAGRIRRWDDAAIRRLNPGLTLPERAIERVVRSDSSGTTWNFADYLAKLSPRWRAEFGVAARFDWGEGVIAAKGSGGVAEAVARTPGAIGYVDYNYVVRHGLQAVTLQNRDGAFVQAGTDGFAAALSASPWPRSGDFTATLTDQPGARSWPITMGTFILVPRHGGGDGVRRALAFFTWAYLHGDELIRSSHFVRLPDRVQAKAFRSLAAVTDANGTPIGFGGLAR

SEQ ID NO: 408 - protein, 664 aa, Stutzerimonas stutzeri
MITENNDILFGNGDELPSNHSTNPHIQDVISLGRRKVLAGGAAMGALAFLGASLPGLAQAAEPMSRGIKDVPFKRRTRLPFVPVAVTRADTITVPSGYTATTFIPWGTPITGRYPAWLEDASNTAEDQAQQVGMHHDGMHFFPMNAKLGGRQSDHGLLVLNHEYIDAPLLHRNGPTVVDGKRVNVDEVRKEINAHGVSVVEIRRGPRGEWSVLPTARNRRITGATPMRIEGPARGHALMRTRYSPSGTSVRGTLNNCANGFTPWGTYLTCEENWAGYFATGDAELPRELSRYGVRGAGRYGWETVAGDEFERFNATRIAATAQGDYRNEPNTFGWVVEIDPFDPHSTPIKHTALGRFAHEGLVFAPVKPGRPVVCYSGDDSQNEYIYKYVSRDKYRPQRSDGRLLDDGVLYVARFNPDGSGDWLALDHANPAFQRACKAAGVRFADQGEVLINTRLAADIVGATKMDRPEWGAVHPDTGEVYFTLTNNSGRQQSDAANPRSPNAFGHIIRWREASRDFAGTRFTWDIYLLAGPEENSRSPKGRALDATNILASPDGLWFDDEGRLWIQTDMSGSQLSSGPFGNNQMLVSDPRTGETKRFLVGPPGAEVTGITATPDFRTLFVNIQHPGEGSTSTNYTSTWPDGAGRRPRSATVIITREDGKRLM

SEQ ID NO: 409 - skipped

SEQ ID NO: 410 - protein, 672 aa, Pseudomonas aeruginosa
MSLEKKDAILFGDGDELPSNHSNNPHMNDLIAGLGRRQVLAGGAALGALAFLGVALPASAAPAEAQVPAAEGLRGLPFKRRNRLPFEAIASGRADTIRVPAGYKATPFIPWGTPISGSYPGFLDDASNSAQDQAEQIGMHHDGMHFFPIDAQFGFGGGHISNHGLLVLNHEYIDAPLLHTNGPTVIDGKRTVADEVRKEINAHGVSVVEIRRNVRGEWNVVPSQRNRRITAATPMRIAGPARGHELLRTRHSPDGTRTRGTHNNCSNGFTPWGTYLTCEENWVGYFSTQDSELPRELTRYGIRNTSRFGWETLAGDEFERFDATRKGKQAQDDYRNEPNNFGWIVEIDPFDPQSVPVKRTALGRFAHEGLVFAPVKPGRQVVCYSGDDSQNEYIYKYVSRDKFRPGRSNTRLLDEGTLYVARFNADGSGQWLPLDIADGNFRAACRKAGVSFADQGEVLINTRLAADVLGATKMDRPEWGAVNPDNGDVYFTLTNNTSRTVADAANPRVKNAYGHIIRWRERSRDYAGQRFTWDLFMLAGPGEDSRGPDGKPLNQDNILASPDGLWFDPEGRLWIQTDMSGSQLSSGPFGNNQMLVADPRTGELKRFLTGPLGCEVTGIAATPDFRTLFINIQHPGEGSTADNLLSTWPDGPGRRPRSATVVITREDGRRLL

SEQ ID NO: 411 - protein, 651 aa, Aromatoleum aromaticum
MVERVVSRRGFLKNGIGLGAAAFPAGPLSAFAKPAGTPALHQKTPTIGFTPMAVSSGDAVVVPSGYRAQVLARWGEALFADSPEWLPDGSNTGEDQARQIGDNHDGMHFFPLNGQSSKEGLLVVNHEYTNYEYLFAPEPGQPYLEPWTLDKVRKAQHAHGVSVLHIDQKSGKWEIVRDSKYNRRITGNTPMAISGPASGHPLMRTSADPEGVEVLGTLNNCANGFTPWGTYLACEENFQGYFGTTSGIDARDDVMRRYGVSPLGSGYRWEEHDDRFDYVKEPNESNCFGWIVEIDPFDPDSVPVKHTALGRIKHENAALTVAGTGRVVVYMGDDQTNDYIYKYVSDDRFIPGRKTANSELLDHGRLYVAKFSDGAVSGDFIGTGEWILLDKHANPILAADPRFVDQGELLIKTRLAADAVGATKMDRPEWITVHPGSGEVYCTLTNNSGRAAANIDDANPRAANSWGQIVRWREDGDAAEATTFEWDLFVVAGNPIAFPERADLRSGSEQITADNTFNSPDGLGFDSEGRLWIQTDGNFSNTGNYLGQGNNQMLCADPQSGEIRRFLVGPSGCEITGIAWTPDLKTMFVNVQHPGEVGNHPNRPVLPTGVSMDEYMAANPLAFSQWPEAAGGRPRSATVVITKEDGGVIGT

SEQ ID NO: 412 - protein, 629 aa, Yoonia vestfoldensis
MKDIDTKTLSWDEWDELQRPALATTDFDAVVDTALSRRGFLGGVLAFGSGAAVMGTGLLSGTSAQAQATSRFGFNAIPIATDFTVHVPEGYTWQPVAKWGQPLFSDAAPLSQETGVDLASSDRVFGENTDGMELFSIGGRQVVAVNHEYVNPDINLPHTGGETTSADDARILQNMQGVTVMEVAEGANGWDIVVDSPFNRRITQNTPMTFSGPAAGHPLLQTAADPDGMIVLGTFNNCGSGKTPWGTYLTCEENFNGYFGSTDAAFARPDDYVRYGIGLESRYGYEKFDARFDVSQNVNEPRRQGYVVEIDPSDATSTPVKHTALGRFKHENAAVVVARDGRVVVYLGDDERGEFLYKFVSAGVYTPGGETSSLLSDGQLYVARFATDMTGEWVALTPEATGMDAAEIAVYTRVAASRVGATTMDRPEWVAVNPVAIEGYCALTNNSRRGADATNAGGDAMQAVENSPNPREANRYGQILRWFPDAEDHAATTFHWDLYVMAGNPTVHQDANAGSANINEGNLFNSPDGMMFDSSGLLWIQTDGDDSNEEGFAGMGNNQMLAGDPVTGEIARFLTGPNGSEVTGLTWSADRRTMFVGIQHPAAPFPDGDGKLARSTVIAVKKADGAVIG

SEQ ID NO: 413 - protein, 620 aa, Maritimibacter alkaliphilus
MKDQDTSNLSWDEFDEMRDPRPATNDFDAVVERAITRRGFLGGALAFGSAATVFGSAMLTSSRAQAAAHGGFGFEPIDIATDFDIHVPEGYEWKTLVRWGDALWSEADGAYDAETGVSPAMSDKVFGENTDGMETFFVDGKELIAVNSEYVNPKINLPAASDGTPQNADEVMLLKNLQGVTVMEVAMNGGTYEVVKDSPYNRRITHETQMTMDGPAAGHALVQTNADPEGMSPKGTMNNCGSGKTLWGTYLTCEENFNGYFGATGGYDETDGLSRYGIGGEGRYAYEKFDPRYDLTAEPNEPNRHGWITEIDPTDPESTPVKHTALGRFKHENAEMVQAADGRVVVYMGDDERGEFIYRYVSNGTYAEGGSTEGLLSDGQLYVAKFNDDMTGEWIALTPEATGMSLEETLVFSRIAASAVGATSMDRPEWIAANPLKAEAYCALTNNSKRGGDAMPVNAANPRETNEYGQIVRWAPAGDDHGADTFAWDLYVMAGNPAVYDNAYGGSENVTDGNMFNSPDGMAFDSKGMLWIQTDGDDSNEGDFAGQGNNQMLVGNTETGEIARFLTAPNGAEVTGLCWNEDNTVAFVGIQHPGGSWPDGNGKPRSSIIQVWRTDGAPVG

SEQ ID NO: 414 - protein, 382 aa, Bacillus subtilis; identical to SEQ ID NO: 57

SEQ ID NO: 415 - protein, 349 aa, Herbaspirillum seropedicae
MGFNQLIKSVAVAVAGAFAFSGAAHAAEITGAGASFPYPVYAKWAELYKAATGNSLNYQSIGSGGGIKQIKAKTVDFGASDAPLPQNELAEAGLTQFPAVVGGVVPVVNLDGVTPGQIKLSGQVLGDIYAGKITKWNAPEITALNPGVKLPNEDITVVYRSDASGTSYVFTSYLSKANADFKSKIGAGTAVKWPTGVGGKGNEGVAANVQKVKGAIGYVEYAYVKKNKLNYTQLKNKDGNFVSPDDSSFKAATAHGDWAKTPGFAVDFTDAAGKESWPISSATFILLHKEQAGDGAKGKEVLKFFDWAYKNGGKVAADLDYVPLPDSVTKLVEDSWKSNVKDSSGKAIW

SEQ ID NO: 416 - protein, 334 aa, Gluconacetobacter diazotrophicus
MRRSVTVFAAVLLGTAPVALSVAHAANITGAGSSFAAPIYEAWGSAGRSASGVAVNYQSVGSSAGQNQILAGTVDFGASDAPMDPAKLQKGALFQFPTVMGGIVPVVNIPGIAANTLRLTGEVLAGIYSGDISSWNDAKITALNPGVNLPDLPIATVHRADGSGTTFVFTSYLARSSQAWHDGTGAGSSISWPGGVGARGNDGVAASVRNTEGGIGYVEYAYASRNHMTTVQLKDKAGDFVTADLDSFSRAAAAADWKGAANFAVDLLDTSGKGAWPIVSATYVLVPKQAKDAAQGAAVRKFFGWAFDNGDGIARGLDYVVLPAEVKASIRAAW

SEQ ID NO: 417 - DNA, 508 bp, Azorhizobium caulinodans
  source 1..508: mol_type = genomic DNA
gggatgaactcatccgcctgttcgagaatctggtggagaacgccctgaaatacggcggcaccggccagaacgtggatgtgcgcatcgcccgcgagccggggacgcagggtgacgtggctgcggtgagcgtgcgggactatgggccgggcatcgcgccggagcatctgccccgcctgaccgagcgcttctatcgggtggacgttgcggcgagccgcgaccagggcgggacgggtctcggcctcgccatcgtcaagcatatcgtcgcccgccatcggggccggctgatggtggagagcacccccggaaagggggcaacttttaccgtgcgcctcgacctctccgatgacgctgcgaaaaactcagtaaatacagcggcttgatccgtcattcaggtgtcacgaaccagtcatagaaggttcgggccagaccgctatgtgtgggcggtccaaagttgcgtcaggacgggaacgtcccgtggcagcacctgattggcaatggagagaacccc

SEQ ID NO: 418 - DNA, 192 bp, Azospirillum brasilense
  source 1..192: mol_type = genomic DNA
aggttgcggttaccccccatcggcgtgaccgaaagtgggcgtcgtcatgaaactgtcataaaaccgtaatcagcgcgtcgcggaccccgactagtgtccgccccgcaagccgcccgcccctgggggaagcggcacggggttgaaccatcgcgttccccggcaaaggggcaaccggaccatcggagggatacc

SEQ ID NO: 419 - DNA, 100 bp, Escherichia coli
  source 1..100: mol_type = genomic DNA
aaagttaatcttttcaacagctgtcataaagttgtcacggccgagacttatagtcgctttgtttttattttttaatgtatttgtacatggagaaaataaa

SEQ ID NO: 420 - DNA, 563 bp, Escherichia coli
  source 1..563: mol_type = genomic DNA
tattgagtgtgaaaacctggggatactgaataccgatgatatcagtacgtctttttatgctaccgaacccaatacggacaattcaatggtcgtaacttcaaactctaacgtggggataaaaatttacgacaaaaataataaggaaatcaaagtgaacggtggtgagttgccaacagacatgggtaaatcaacagtgtatggtgaaaaatcaggtagtgtaactttttcagctgctcctgcgagccttactggagctcgcccagccccggggcaatttactgcaacagcgacgataactgttgagattgtgcgctaataggtctgacaacgattgttcatcacatcgttgtcatctcacccatttctttatcacttttaaatgcatcatgacaaaaagatgacatgtgggattttcatcttcacatttaaaatcagtaagttatttggtaattcctttctgtcataaaaatgtcatattccttacatataactgtcacctgtttgtcctattttgcttctcgtagccaacaaacaatgctttatgaatcctcccaggagacatt

SEQ ID NO: 421 - DNA, 386 bp, Gluconacetobacter diazotrophicus
  source 1..386: mol_type = genomic DNA
tcgtttccggggggatgatcccgacgggtcctgtctggcggctgcccccgccgccgggcaggacctccggccgttttcggccacatgtgtcgttgttaataagaaacgatctcattccctgccgatcgtggcgcggatgcgccatgacccggggtcgcatagggcatcgggatatgccggcctgcgccgtctcgtatcatttcaagcgcggttttacttgccgaatggctgccatgtgcggaatgtagagaaaagagggcggaccatgattgcagtacggatgtgaagtaagttagcattagggtccagttcgaccgttccggtcggataacgccgtcttgcgggtctctggaccacgccgggaataaagaagaggaaaggtggcg

SEQ ID NO: 422 - DNA, 457 bp, Herbaspirillum seropedicae
  source 1..457: mol_type = genomic DNA
caagagtccctgtccatccagtttgagcgggcagtgtattgaggaggtatgacgagtttatgacaagtattatgacgctttgtcattttttctcagcagaaaatatcatattttcgatgcaatgaatgcaagatccctgtccccgtcagctgactgaccaacgctgcaacagaactgtcaggaaagcccctccagccccagcctgcgcccctttccgagctgcttgatggtgctcggaatactttcctgcaggaaattatgacctgcccgaagatgacaagtttgggtcataaactcgtcacattagctggttaaagttcgccctgtctcagaagtcatccctcccaccgtgatgtttcttgctgacgtcctctctcaaaatcataggagtagttgaaatggggtttaaccaactgatcaaatctgttgcagtcgcagtggccggcgctttcgcttt

SEQ ID NO: 423 - DNA, 496 bp, Kosakonia radicincitans
  source 1..496: mol_type = genomic DNA
gttcgccgatcaggatgcgggtttcaccagcaacgacaatatgcacattattgatatgccgcacgtcgaagaggtgattgcaccgattttctataccgtgccgctgcaattgctggcttatcacgttgcgctgataaaaggcaccgatgttgaccagccgcgtaacctggcgaaatcagtaacggtagagtaaacatttactgccataaaatgcgaaagaggttgccggcgggcaacctcttttttttactttattttgtgaattctcattcgtttcaactacttttttattgtcacaaaaataaaatatgactgaaatattactgtgatgttttttattaagagtttgatttttatcttttttatatcatgcgataacgatatttttccgctgtcatcaaacagtaacaatgattgcatataactgtcatcaaagtgtcctattttgctcgtcgtagccactaaacaacgatttacgatccttgcaggagacatt

SEQ ID NO: 424 - DNA, 139 bp, Kosakonia radicincitans
  source 1..139: mol_type = genomic DNA
tgcccggaggtttttttctgctgattgataccgctctattttttgtcatgacacaaaattgtcatataagcgctctatccttccctccgtccgcaaaattattcaaaaatctatcaactttttcgtttttggagagatt

SEQ ID NO: 425 - DNA, 489 bp, Paenibacillus azotofixans
  source 1..489: mol_type = genomic DNA
tggcggcgtctatctgaatgcgataggcggagcggcgcagtattacgccgaatgtatcaagaaagtgaacggcgtcgattttctcgaattcggtattcccgaggcgatgtggcatctgacgatagacggatttgcggcgattgtgaccatggattcccacggcaatagcctgcatgccgacgttgagaaagactccgccagtaaattggcgttattccgtgaagcggtgtttaagtaaagtgtcatatggacgatgatccgtgtctagaagtgctttttcgcgaaagcttggctattttggtgtgtagcctggacggcagcttttagcaatgatagtgatcatatgaagcggatcggagcacagatgtatacgtctggagctccgatttttggttttgcatggtatgcttttgcactggcaatttcactgaaatcgggctacgatagagtaaagcagtatagtaaagcatagaaagaggcatgtgccgc

SEQ ID NO: 426 - DNA, 437 bp, Pseudomonas stutzeri
  source 1..437: mol_type = genomic DNA
cctgatcaccctgccgacctaccacactgctgctctgtcgaccgacaacctggccaagggctactttgccgaccagggcatgctggcctacgtcaagggcgttcagcgcgaggaactgcgtcagggtatcgcctgcgtcaagcaccagaacatggctggttctgacatcggcgacaaccacaaggagtacttcgctggcgaagctgctctgaaggcaagcggcaaagacaacaccatgaaccagttccactaagaacaggttcgcagccgccccggctgcggtgtgaaaaaagccctggcgtgagccggggcttttttttgcgacggattgagtggtgcacggttacgcttcgtcggtcagcgctccactgtcattctcttgtcgcatacgaaccttagcgtcatggctccgacaacaatcgttatggagctgcgcc

SEQ ID NO: 427 - DNA, 848 bp, Pseudomonas stutzeri
  source 1..848: mol_type = genomic DNA
cgccctgctgggcgagtcactttttccagacgcggaaaaagtaaccaaaaacgctttgcccctgcatccggccccgcgctccgcgcggggttcgttcgctccatcgccgcttcaggggtcggcttacaagggccttccatggccctttaagcctttcgccgcatccatgcggctcacccccttacgcaacgattccgctctccctactgaaggggcgaggctggggcctgcgacgattttcaacagacggatgatgacgatcagccgacccgtgtcgttcgttggaaacgtcctttacggttttccctcatattggatcctagcaatcacggacttgcaggcgacatcgatcgcccctttcaggaggccgaatgtagtcgctacgtagcggggcgagcggcatggatgccgcgagagaactaggcgtccccccgtaaagggccatggatggcccttgcacggtgaccctcggagaagcgatggaatgagggaagtctcgcgtagcgagacccggatgtaggggcaagactttttggttccttttggcggggccggccatccgggcgactgccaaaaggaactcgcgcagcagcgcgaaacagaagtaccggaacacccggaaaatgggttgctcccccaacctgatcccagcgtgtctgcgataggcatttccatatccataccgctaaaaacaggtcatatcagctcaaccccacccccaaaatcctgcaaaacccactcttccaagctctccccaaatttcctgtcacagaaccgccacatcccccacctaccgtccgctgcagttccgggatggacccggcaaacaccattcgggggaatcgttc

SEQ ID NO: 428 - DNA, 1062 bp, Pseudomonas stutzeri
  source 1..1062: mol_type = genomic DNA
ccgccagccgctgagcctgctgttgctcgatctggatgatttcaaggcctacaacgataccttcggccatccggccggcgatgcgctgatccgttcattcggcgagctggtggcaaagttcgcccgacggccgttggatcaggctgcgcgggtcggtggcgaggagttcgcgctgctgctgtacaactgcgacaacgcggcggcgcaacgcatcgcgcggcagctggtggccgcgctggcggcgctggaggtcaggcatccgcgcgatccggcgtcgcgcgtgggggtcagcatcggcgtggccacgctgcaggatggccagcaggccgagcagctctatcacaacgccgacatggcgctgtaccaggccaagacgaccggcaagaaccgcttcgcggtcgcccccgggcaggcgcgccgcgactagctggccaacctcagcggcgcggcgggaccgaacgcgtgcggccgctgccgtcgatggcgacgaagacgaacaccgcttcggtgaccttgcgccattcgctggacagcgggtcgtcgctccagacttccaccagcatgcgaatcgagctgcgtccgacttccagcgtctgggtatagaaggacagctgcgcgccgacggccaccggcaccatgaaggccatgcgatcgatggacaccgtggccacgcggccggccgccacgcggctggccatcgcagtaccggccaggtccatctgcgacaccagccagccgccgtagatatcgccgaacccgttggtttcgcgcggcaatgcggtgagctgcagggccaggtcgccctggggaatcggatcttcctgttcgtattctttcatcggtcggactcgcggcgcgtttgttgttctggcgcgaaatgcccgcgctagcgcgggcgacggcgccgagtatagcggcagtgcagacgtacagcgaccgcgctgggtcaatgggccggttcgtaatcagattgtcacacttgcttcatagagtgttcacacggcctgcagatactggcccccgttccaacacactcgagcacacacctgctaggagcaaggt

SEQ ID NO: 429 - DNA, 88 bp, synthetic construct
  source 1..88: mol_type = other DNA
gggcccaagttcacttaaaaaggagatcaacaatgaaagcaattttcgtactgaaacatcttaatcatgcacaggagactttctaatg

SEQ ID NO: 430 - DNA, 88 bp, synthetic construct
  source 1..88: mol_type = other DNA
gggcccaagttcacttaaaaaggagatcaacaatgaaagcaattttcgtactgaaacatcttaatcatgctaaggaggttttctaatg

SEQ ID NO: 431 - DNA, 88 bp, synthetic construct
  source 1..88: mol_type = other DNA
gggcccaagttcacttaaaaaggagatcaacaatgaaagcaattttcgtactgaaacatgttaaccatgctaaggaggttttctaatg

SEQ ID NO: 432 - DNA, 88 bp, synthetic construct
  source 1..88: mol_type = other DNA
gggcccaagttcacttaaaaaggagatcaacaatgaaagcaattttcgtactgaaacatcttaatcatgcaggggagggtttctaatg

SEQ ID NO: 433 - DNA, 88 bp, synthetic construct
  source 1..88: mol_type = other DNA
gggcccaagttcacttaaaaaggagatcaacaatgaaagcaattttcgtactgaaacatcttaatcatgcgccggaggttttctaatg

SEQ ID NO: 434 - DNA, 88 bp, synthetic construct
  source 1..88: mol_type = other DNA
gggcccaagttcacttaaaaaggagatcaacaatgaaagcaattttcgtactgaaacatcttaatcatgctggggagggtttctaatg

SEQ ID NO: 435 - DNA, 88 bp, synthetic construct
  source 1..88: mol_type = other DNA
gggcccaagttcacttaaaaaggagatcaacaatgaaagcaattttcgtactgaaacatcttaatcatgcatcggaccgtttctaatg

SEQ ID NO: 436 - DNA, 88 bp, synthetic construct
  source 1..88: mol_type = other DNA
gggcccaagttcacttaaaaaggagatcaacaatgaaagcaattttcgtactgaaacatcttaatcatgcagaggagtctttctaatg

SEQ ID NO: 437 - DNA, 88 bp, synthetic construct
  source 1..88: mol_type = other DNA
gggcccaagttcacttaaaaaggagatcaacaatgaaagcaattttcgtactgaaacatcttaatcatgcggaggatcgtttctaatg

SEQ ID NO: 438 - DNA, 88 bp, synthetic construct
  source 1..88: mol_type = other DNA
gggcccaagttcacttaaaaaggagatcaacaatgaaagcaattttcgtactgaaacatcttaatcatgcgggggagtgtttctaatg

SEQ ID NO: 439 - DNA, 88 bp, synthetic construct
  source 1..88: mol_type = other DNA
gggcccaagttcacttaaaaaggagatcaacaatgaaagcaattttcgtactgaaacatcttaatcatgctgcggagggtttctaatg

SEQ ID NO: 440 - DNA, 88 bp, synthetic construct
  source 1..88: mol_type = other DNA
gggcccaagttcacttaaaaaggagatcaacaatgaaagcaattttcgtactgaaacatcttaatcatgcaatggaggctttctaatg

SEQ ID NO: 441 - DNA, 88 bp, synthetic construct
  source 1..88: mol_type = other DNA
gggcccaagttcacttaaaaaggagatcaacaatgaaagcaattttcgtactgaaacatcttaatcatgcggtggagggtttctaatg

SEQ ID NO: 442 - DNA, 88 bp, synthetic construct
  source 1..88: mol_type = other DNA
gggcccaagttcacttaaaaaggagatcaacaatgaaagcaattttcgtactgaaacatcttaatcatgcgggggagtctttctaatg

SEQ ID NO: 443 - DNA, 88 bp, synthetic construct
  source 1..88: mol_type = other DNA
gggcccaagttcacttaaaaaggagatcaacaatgaaagcaattttcgtactgaaacatcttaatcatgcttaggagtctttctaatg

SEQ ID NO: 444 - DNA, 88 bp, synthetic construct
  source 1..88: mol_type = other DNA
gggcccaagttcacttaaaaaggagatcaacaatgaaagcaattttcgtactgaaacatcttaatcatgcggaggagggtttctaatg

SEQ ID NO: 445 - DNA, 88 bp, synthetic construct
  source 1..88: mol_type = other DNA
gggcccaagttcacttaaaaaggagatcaacaatgaaagcaattttcgtactgaaacatcttaatcatgcgacggagcgtttctaatg

SEQ ID NO: 446 - DNA, 88 bp, synthetic construct
  source 1..88: mol_type = other DNA
gggcccaagttcacttaaaaaggagatcaacaatgaaagcaattttcgtactgaaacatcttaatcatgctatggaggttttctaatg

SEQ ID NO: 447 - DNA, 88 bp, synthetic construct
  source 1..88: mol_type = other DNA
gggcccaagttcacttaaaaaggagatcaacaatgaaagcaattttcgtactgaaacatcttaatcatgctgaggaaagtttctaatg

SEQ ID NO: 448 - DNA, 88 bp, synthetic construct
  source 1..88: mol_type = other DNA
gggcccaagttcacttaaaaaggagatcaacaatgaaagcaattttcgtactgaaacatcttaatcatgcgagggatggtttctaatg

SEQ ID NO: 449 - DNA, 88 bp, synthetic construct
  source 1..88: mol_type = other DNA
gggcccaagttcacttaaaaaggagatcaacaatgaaagcaattttcgtactgaaacatcttaatcatgcctaggaagttttctaatg

SEQ ID NO: 450 - DNA, 85 bp, synthetic construct
  source 1..85: mol_type = other DNA
gggcccaagttcacttaaaaaggagatcaacaatgaaagcaattttcgtactgaaacatgttaaccatgcctaggaagttttcta

SEQ ID NO: 451 - DNA, 88 bp, synthetic construct
  source 1..88: mol_type = other DNA
gggcccaagttcacttaaaaaggagatcaacaatgaaagcaattttcgtactgaaacatcttaatcatgcgaaggacagtttctaatg

SEQ ID NO: 452 - DNA, 88 bp, synthetic construct
  source 1..88: mol_type = other DNA
gggcccaagttcacttaaaaaggagatcaacaatgaaagcaattttcgtactgaaacatcttaatcatgcgatggacggtttctaatg

SEQ ID NO: 453 - DNA, 15 bp, synthetic construct
  source 1..15: mol_type = other DNA
ttcacacaggaaacc

SEQ ID NO: 454 - DNA, 15 bp, synthetic construct
  source 1..15: mol_type = other DNA
attaaagaggagaaa

SEQ ID NO: 455 - DNA, 12 bp, synthetic construct
  source 1..12: mol_type = other DNA
aaagaggagaaa

SEQ ID NO: 456 - DNA, 34 bp, synthetic construct
  source 1..34: mol_type = other DNA
cgcaaaaaaccccgcttcggcggggttttttcgc

SEQ ID NO: 457 - DNA, 39 bp, synthetic construct
  source 1..39: mol_type = other DNA
aaaaaaaaaccccgcccctgacagggcggggtttttttt

SEQ ID NO: 458 - DNA, 129 bp, synthetic construct
  source 1..129: mol_type = other DNA
ccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 459 - DNA, 42 bp, synthetic construct
  source 1..42: mol_type = other DNA
gttgtcacatatctgtcatcgcggccaggtataattgcacga

SEQ ID NO: 460 - protein, 481 aa, Acetobacter malorum
MVKKTTTAGDSKAAAIAKVFDLIQENSVEFVDLRFTDPRGKWHHTTQYVTTIEEDTFTEGFMFDGSSIAGWKAINESDMVLLPDPETAVMDPFTARPQLILFCDIIEPSTGQPYNRDPRSTAKLAEQYLKSSGLGDTAFFGPEAEFFIFDNVRFGTGPNFGKYQLDSIEGPGASLKDYPEGNMGHRPGVKGGYFPVPPVDSENDLRAEMLATMGEMGLPIEKQHHEVAQSQHELGTKFDTLVRSADFMQIYKYCVHNVAHSYGKTATFMPKPIYGDNGSGMHVHQSIWKDGKPTFAGNGYADLSDTALYYIGGIIKHAKALNAFTNPSTNSYKRLIPGFEAPVLLAYSARNRSASCRIPYATNPKAKRVEVRFPDPTANPYLAFSAMLMAGIDGIKNKIHPGDAMDKDLYDLPPEELKQIPTVAGSLREALEALAADHEFLLAGGVFTKDQIESYLTVKWEDVYRFEHTPHPVEFEMYYSV

SEQ ID NO: 461 - protein, 481 aa, Acetobacter tropicalis
MVKKTTSASDAKAAAIAKVFDLIQENSVELVDLRFTDPRGKWHHTTQYVTTIEDDTFTEGFMFDGSSIAGWKAINESDMVLLPDPETAVMDPFSARPQLILFCDIIEPSTGQPYNRDPRSTAKLAEQYLKSTGLGDTAFFGPEAEFFIFDNVLFGTGPNFGKFQLDSIEGPGASLKEYPEGNMGHRPGVKGGYFPVAPVDSESDLRAEMLATMGEMGLPIEKHHHEVAQSQHELGTKFDTLVRSADFMQIYKYCIHNVAQSYGKTATFMPKPIYGDNGSGMHVHQSIWKDGKPTFAGNGYADLSDTALYYIGGIIKHAKALNAFTNPSTNSYKRLIPGFEAPVLLAYSARNRSASCRIPYATNPKAKRVEVRFPDPTANPYLAFAAMLMAGLDGIKNKIHPGDAMDKDLYDLPPEELKQIPTVAGSLREALEALQADHEFLLAGGVFTKDQIESYINIKWEDVYRFEHTPHPVEFEMYYSV

SEQ ID NO: 462 - protein, 481 aa, Acetobacter pasteurianus
MVKKTAPAGDSKAAAIAKVFDLIQEHSVELVDLRFTDPKGKWHHTCQYVSTIEEDSFTDGFMFDGSSIGGWKAINESDMVLLPDPTSAVMDPFSARPQLILFCDIIEPSTGQPYNRDPRSTAKLAEQYLKSSGLGDTAFFGPEAEFFIFDNVLFGTGPNYGKYQLDSIEGPDASLKEYPEGNMGHRPGVKGGYFPVPPVDSESDLRAEMLAAMGEMGLAIEKHHHEVAQSQHELGTKFETLVRAADFMQIYKYCVHNVANSYGKTATFMPKPIYGDNGSGMHVHQSIWKDGKPTFAGNGYADLSDTALYYIGGIIKHAKALNAFTNPSTNSYKRLIPGFEAPVLLAYSARNRSASCRIPYATSPKAKRVEVRFPDPTANPYLAFAAMLMAGMDGIKNKIHPGDAMDKDLYDLPPEELKQIPTVAGSLREALEALAADHEFLLAGGVFTKDQIESYITLKWDDVYRFEHTPHPVEFEMYYSV

SEQ ID NO: 463 - protein, 474 aa, Acidothermus cellulolyticus
MFRNADEVLAFIRDNDVKSVDVRFCDLPGIMQHFTVPAESFDESVFTDGLAFDGSSIRGFQAIHESDMQLLPDPTTAFIDPFRTEKTLAMNFFIHDPLTGEAYSRDPRNVARKAEAYLVSTGIADTAYFGPEAEFYIFDEVRFETSAHQSYYHIDSVAGAWNTGRIEDNGNRGYKVRYKGGYFPVPPTDHFADLRAEMARELIRAGILVEKQHHEVGTAGQAEINYKFNTLLRAADDMQLFKYIVKNVAWRAGKTVTFMPKPIFGDNGSGMHTHQSLWKDGSPLFYDELGYAGLSDTARYYIGGLLRHAPSLLAFTNPTVNSYHRLVPGFEAPVNLVYSQRNRSACIRIPITGTNPKAKRLEFRVPDPSCNPYLAFAAMLMAGLDGIKNKIEPPEPIDKDLYELPPEEHAAVPQVPGSLPEVLAALEADHDYLLEGGVFTPDLIATWLEYKRTKEVDSIRLRPHPHEFELYFDV

SEQ ID NO: 464 - protein, 471 aa, Acinetobacter baumannii
MSMANKVLQLIQESGAKWVDFRFTDTKGKEQHVTYPADSIDEDTFEDGKMFDGSSIAGWKGIEASDMILRPDAETGFIDPFFAEPTVVVTCDVIEPSTGHGYERDPRSIARRAEEYLKSTGIGDTAFFGPEPEFFVFDEVKWDIDMSGARHTLIAEEAAWSTGKDYESGNSGHRPRVKGGYFPVPPVDSAQDMRAEMCAKIEDIMGPGRVEVHHHEVASCQLEIGVSFNTLVRKADEVQQFKYAVWNVAHQYAKTATFMPKPMVGDNGSGMHVHMSISKDGKNLFAGDEYAGLSEMALYFIGGIIKHARALNAITNPSTNSYKRLVPHFEAPIMLAYSARNRSASIRIPYVSNPKGKRIEARFPDPMMNPYLGFAALLMAGIDGIQNKIHPGEAADKNLYDLPPEEEVKIPTVAHSLDMALEALQADHEFLLKGGVFTKEMLDAYIELKTEDVRRLNTTTHPVEFDMYYSL

SEQ ID NO: 465 - protein, 471 aa, Acinetobacter calcoaceticus
MSMANKVLQLIQESGAKWVDFRFTDTKGKEQHVTYPADSIDEDTFEDGKMFDGSSIAGWKGIEASDMILRPDAETGFVDPFFAEPTVVVTCDVIEPSTGQGYERDPRSIARRAEEYLKSTGIGDTAFFGPEPEFFVFDEVKWDIDMSGARHTLIAEEAAWSTGKDYESGNSGHRPRVKGGYFPVPPVDSSQDMRAEMCAKIEDIMGPGRVEVHHHEVASCQLEIGVSFNTLVRKADEVQQFKYAVWNVAHQYAKTATFMPKPMVGDNGSGMHVHMSISKDGKNLFAGDEYAGLSEMALYFIGGVIKHARALNAITNPSTNSYKRLVPHFEAPIMLAYSARNRSASIRIPYVSSPKGKRIEARFPDPMMNPYLGFAALLMAGIDGIQNKIHPGEAADKNLYDLPPEEEAKIPTVAHSLDMALEALQADHEFLLKGGVFTKEMLDAYIELKTEDVRRLNTTTHPVEFDMYYSL

SEQ ID NO: 466 - protein, 344 aa, Agrobacterium deltaense
MTKFKLEYIWLDGYKPVPNLRGKTQIKEFDEFPTLEQLPLWGFDGSSTQQAEGHSSDCVLKPVAIYPDPARSNGALVMCEVMMPDGVTPHASNSRATILDDEDAWFGFEQEYFFYQDGRPLGFPEQGYPAPQGPYYTGVGFKNVGSVAREIVEEHLDLCLEAGINHEGINAEVAKGQWEFQVFGKGSKRAADQIWIARYLLLRLCEQYGIDVEFHCKPLGDTDWNGSGMHCNFSTKFMREVGGKDYFEALMAAFAKNWKEHIDVYGPDNHLRLTGKHETAPWNKFSYGVADRGASIRVPHSFVNNGYKGYLEDRRPNSQGCPYQIASVVLKTIAEVPLAKSAAA

SEQ ID NO: 467 - protein, 469 aa, Aromatoleum aromaticum
MNAQEVMKMIQENEVRFVDLRFTDTRGKEHHVGLPVSAFEEDHFEHGHPFDGSSLAGWKGIQASDMILLPEAGSAYIDPFFDETTVVLTCDVIEPSDGKGYDRDPRSIAKRAEAYLKSTGVGDTAFFGPEPEFFIFDSVEWSVDMSGVYSKIISEEAAWSTADKFEGGNTGHRPRIKGGYFPVPPVDSLNDIRAAMVLALEACGVPVEVHHHEVANAGQCEIGTKFSTLTKRADWTQVLKYIVHNVAHQYGKTATFMPKPIVGDNGSGMHVHQSIWKDGQNLFAGNGYAGLSEMALYYIGGIIKHARALNAITNPGTNSYKRLVPHYEAPTKLAYSARNRSASIRIPYVANPKGRRIEARFPDPLANPYLAFSALMMAGLDGIQNKIHPGDPADKNLYDLPPEEDAKIPTVCTSLDQALEYLDNDREFLTRGGVFSNDFIDAYLALKGEEVDRMRITTHPVEFDMYYSL

SEQ ID NO: 468 - protein, 446 aa, Arthrobacer sp.
MDRQQEFVLRTIEERDVRFVRLWFTDVVGSLKSVALAPAEVEGAFEEGLGFDGSAIEGLARVFESDMLAQPDPSTFQILPWRGETEQTSRMYCDILTPDGEPSAADPRNVLKRTLAKAADMGFTCYTHPEIEFYLLKSQEPGPDGSPVPVDEGGYFDHVPGGVAQDFRRTAVTMLESVGISVEFSHHEAGPGQNEIDLRYADALQTADNIMTFRTVIKEVALQQGTYATFMPKPFTAHPGSGMHTHFSLFEGDTNAFYEAGAEFQLSKTAKQFIAGILKHAPEFTAVTNQFVNSYKRLWGGGEAPSYLSWGHNNRSALVRVPLYKPGKGQSARIEYRGIDSAANPYLAYAVLLGAGLKGIEEGYEIPAAAEDDIWSLSSAERRAMGHDPLPASLHDAIRTMEDSELMPQILGEQVFEYFLRNKRAEWQDYRLQVTPYELQRNLGIL

SEQ ID NO: 469 - protein, 469 aa, Azoarcus sp.
MNAQDVMKMIQENEVRFVDLRFTDTRGKEQHVGLPVSAFEEDHFEHGHPFDGSSIAGWKGIQASDMILMPEAGSAYIDPFFDETTLVLTCDVIEPSDGKGYDRDPRSIAKRAEAYLKSTGIGDTAYFGPEPEFFIFDAVEWSVDMSGVYSKIISEEAAWSTADKFEGGNTGHRPRVKGGYFPVPPVDSLNDVRAAMVLALEGAGVPVEVHHHEVANAGQCEIGTKFNTLTKRADWTQVLKYIVHNVAHQYGKTATFMPKPIVGDNGSGMHVHQSIWKDGQNLFAGNGYAGLSETALYYIGGIIKHARALNAITNPGTNSYKRLVPHYEAPTKLAYSARNRSASIRIPYVANPKGRRIEARFPDPLANPYLAFAALMMAGLDGIQNKIHPGDPADKNLYDLPPEEDALIPTVCTSLEQALEYLDKDREFLTRGGVFSNDFIDAYIALKMEEVDRMRITTHPVEFDMYYSL

SEQ ID NO: 470 - protein, 469 aa, Azorhizobium caulinodans
MKTAKEVLDFIKSNDVKYVDLRFTDPRGKWQHVTFDITMVDEDFFAEGQAFDGSSIAGWKAINESDMLLMPDVTTACVDPFFSETTLSVVCDVLEPTTGEPYGRDPRGIAKKAMAYLQSTGIGDTVFFGPEAEFFIFDDVKFKADPYNTGFKLDSIELPTNGDTDYEGGNLGHRIKTKGGYFPVPPLDSAQDMRSEMLASMAKMGAKVEKHHHEVASAQHELGLKFGQLVTMADHLQVYKYCIHQVANIYGKTATFMPKPVYGDNGSGMHVHQSIWKDGKPLFAGDKYADLSQECLWYIGGVIKHAKSLNAFTNPLTNSYKRLVPGYEAPVLLAYSARNRSASCRIPYTNNPKAKRVEVRFPDPGANPYLAFSALFMAGMDGILNKIDPGSAMDKDLYDLPPAELKQIPTVCGSLREALESLAKDHDYLLKGDVFQKDFIESYIDLKMQEVARFEMTPHPVEFEMYYSV

SEQ ID NO: 471 - protein, 467 aa, Azotobacter vinelandii
MSKSLQLIKEHDVKWIDLRFTDTKGKQQHVTMPARDVDDDFFEYGKMFDGSSIAGWKGIEASDMILMPDDSTAVLDPFTEEPTLIIVCDIIEPSTMQGYDRDPRAIARRAEEYLKSTGIGDTAFFGPEPEFFIFDEVKYKSDISGSMFKIFSEQAAWNTDADFEGGNKGHRPGVKGGYFPVPPVDHDHEIRTAMCNALEEMGLKVEVHHHEVATAGQNEIGVSFNTLVAKADEVQTLKYCVHNVADAYGKTVTFMPKPLYGDNGSGMHVHMSIAKDGKNTFAGEGYAGLSDTALYFIGGIIKHGKALNGFTNPSTNSYKRLVPGFEAPVMLAYSARNRSASIRIPYVNSPKARRIEARFPDPSANPYLAFAALLMAGLDGIQNKIHPGDAADKNLYDLPPEEAKEIPQVCGSLKEALEELDKGRAFLTKGGVFSDDFIDAYLELKSEEEIKVRTFVHPLEYDLYYSV

SEQ ID NO: 472 - protein, 444 aa, Bacillus subtilis
MAKYTREDIEKLVKEENVKYIRLQFTDILGTIKNVEIPVSQLGKALDNKVMFDGSSIEGFVRIEESDMYLYPDLNTFVIFPWTAEKGKVARFICDIYNPDGTPFEGDPRNNLKRILKEMEDLGFSDFNLGPEPEFFLFKLDEKGEPTLELNDKGGYFDLAPTDLGENCRRDIVLELEEMGFEIEASHHEVAPGQHEIDFKYAGAVRSCDDIQTFKLVVKTIARKHGLHATFMPKPLFGVNGSGMHCNLSLFKNGVNAFFDENADLQLSETAKHFIAGIVKHATSFTAVTNPTVNSYKRLVPGYEAPCYVAWSAQNRSPLIRIPASRGISTRVEVRSVDPAANPYLALSVLLAAGLDGIKNKLEAPAPIDRNIYVMSKEERMENGIVDLPATLAEALEEFKSNEVMVKALGEHLFEHFIEAKEIEWDMFRTQVHPWEREQYMSQY

SEQ ID NO: 473 - protein, 478 aa, Bifidobacterium adolescentis
MTALETKADAEALINKEGIEYVSVRFTDLIGVQQHFTVPASEFLKDAFTDGMPFDGSSVEGFQAINESDMKLVPDVSTAFIDPFRKHKTLDVAFSIVDPLTDEPYSRDPRQVAGKAEAYLKSTGIADTASFAPEAEFFIFDKVRFENSMQRSFYEVDSIEAPWNSGIDTEDDGTPNIAFKNRVKKGYFPVPPIDHTQDLRDDMVANLQKVGLILERSHHEVAGAGQQEINYRFNSLQHAGDDLMKYKYVVHETAALAGKAATFMPKPIAGDNGTGMHCHQSLWKDGKPLFYDEKNYGGLSDLARWYIGGLIKHSSSVLAFTNPSLNSYHRLVPGFEAPVNLVYSARNRSAAIRIPLAGTSPAAKRIEFRAPDPSCNPFLAFSAQLMAGLDGILNHIEPPAPVDKDLYELPPEEHAGIKQVPSSLAEAMDALEEDHDFLTAGDVFTDDLIDTWISIKRGEIDQARLAPTPLEYELYFHI

SEQ ID NO: 474 - protein, 478 aa, Bifidobacterium asteroides
MSELKSKADCEALINQEGVEYVSVRFTDLIGVQQHFTVPASEFLKDAFTDGMAFDGSSVQGFQAINESDMKLIPDPETAYLDPFRRHRTLNVAFSIVDPVTDEPYSRDPRQIAAKAEAYLRSTGIADTASFAPEAEFFIFDKVRFENSMQRSFYEVDSIEAPWNSGVDVEEDGSDNIGFKNRVKKGYFPVPPIDHYQDLRDDMVANLQKVGLILERSHHEVGGAGQQEINYRFNTLLHAGDDLMKYKYVVHETAALAGKAATFMPKPIAGDNGTGMHCHQSLWKDGKPLFYDENGYGGLSDIARWYVGGLIEHASSVLAFTNPSTNSYKRLVPGYEAPVNLVYSARNRSAAIRIPLAGTSPAAKRIEFRAPDPSCNPFLAFSAQLMAGLDGILNHTEPPAPIDKDLYELPPEEHAQIKQVPGSLEEALNALEEDHDFLSEGDVFTDDLIQTWLDLKRGELDQTRLAPTPLEYELYFHI

SEQ ID NO: 475 - protein, 469 aa, Bradyrhizobium diazoefficiens
MKTAKDVLKSIKDNDVKYVDLRFTDPRGKWQHVTFDVSMIDEDIFAEGTMFDGSSIAGWKAINESDMCLMPDPVTATIDPFFAETTMVITCDVLEPTTGEPYNRDPRGIAKKAEAMVKSMGVGDSVFVGPEAEFFVFDDVRFSSDPYNTGFRLDSSELPVNSNTEYEGGNLGHRVRTKGGYFPVPPQDSVQDMRSEMLGAMAKMGVKVEKHHHEVASAQHELGMKFDTLTLMADHMQIYKYCIHQVAHIYGKTATFMPKPVYGDNGSGMHVHQSIWKDGKPVFAGNKYADLSETCLHYIGGIIKHAKAINAFTNPSTNSYKRLVPGYEAPVLLAYSARNRSASCRIPYTASPKAKRVEVRFPDPLANPYLGFAAMLMAGLDGIKNKIDPGPAMDKDLYDLPKEELKQIPTVCGSLREALENLDKDRSFLKAGGVFDDDFIDAYIELKMTEVARFEMTPHPIEFDMYYSG

SEQ ID NO: 476 - protein, 469 aa, Bradyrhizobium frederickii
MKTAKDVLKSIKDNDVKYVDLRFTDPRGKWQHVTFDVTMIDEDIFAEGTMFDGSSIAGWKAINESDMCLMPDPVTATIDPFFAETTMVITCDVLEPTTGEPYNRDPRGIAKKAEAMVKSMGVGDSVFVGPEAEFFVFDDVRYSADPYKTGFRLDSSELPTNSDTEYEGGNLGHRIRTKGGYFPVPPQDSVQDMRSEMLGAMAKMGVKVEKHHHEVASAQHELGMKFDTLTLMADHMQIYKYCIHQVAHIYGKTATFMPKPIFGDNGSGMHVHQSIWKDGKPVFAGNKYADLSETCLHYIGGIIKHAKAINAFTNPSTNSYKRLVPGYEAPVLLAYSARNRSASCRIPYTASPKAKRVEVRFPDPMANPYLGFAAMLMAGLDGIKNKIDPGPAMDKDLYDLPKEELKQIPTVCGSLREALENLDKDRGFLKAGGVFDDDFIDAYIELKMTEVARFEMTPHPVEFEMYYSG

SEQ ID NO: 477 - protein, 471 aa, Burkholderia cenocepacia
MSKTVADVMQLVKDEDVKFVDFRFTDTRGKEQHVSVPVSAFDEDKFESGHAFDGSSIAGWKGIEASDMLLMPDPSAAFVDPFYEESTLVLTCDVVEPADGKGYERDPRSLAKRGEAYLKSTGIGDTAYFGPEPEFFIFDSVQWNTDMSGCFVKINSEEAPWSAGKEFEGGNTGHRPGTKGGYFPVAPVDTFQDMRSEMCLLLEQLGIPVEVHHHEVAGQGQNEIGTKFSTLVQRADWTQWSKYIIHNVAHSYGKTATFMPKPVVGDNGSGMHVHQSIWKDGQNLFAGNGYAGLSELALFYIGGIIKHARALNAITNPTTNSYKRLVPHFEAPVKLAYSARNRSASIRIPHVSNPKGRRIETRFPDPMANPYLCFTALMMAGLDGIQNKIHPGEAADKNLYDLPPEEDAKIPTVCAGLDQALEALDKDREFLTRGGVFTDGMIDAYLALKEQELAKFRMTTHPIEFEMYYSL

SEQ ID NO: 478 - protein, 471 aa, Burkholderia stagnalis; identical to SEQ ID NO: 477

SEQ ID NO: 479 - protein, 445 aa, Conexibacter sp.
MPDQPKTREDVKALVQEHRIRFIRLWFTDILGQLKSFSINSEELDDAFEGGMGFDGSSITGFNAIEESDMIAMPDATTFSILPWRPEEQGVARMFCDVQTPERTPYEGDPRHVLRRAVDRANAMGFETFNVGPELEYFLFKDKNGTEVLDEGGYFDLTTLDAGSDVRRETSLALEQLGIKVEYSHHEVGPSQHEIDMRYADALKMADDCMTYRITVKEYAMKYGWHATFMPKPLFAENGSGMHTHMSLFKDGRNAFFDADDPYFLSDIGKAFIAGQLRHARELSSIFAQWVNSYKRLVPGYEAPVYVAWSRRNRSALVRVPLYHPGKEQATRMELRCPDPACNPYLTFAALLHAGLEGIEKGYELPEPMEKNLYHLSPDDRRRLGIEQLPETLGEAIELTAESELVLRTLGEHMFNRYIEIKRQEWEDYRVQVTQWELDRYLPVL

SEQ ID NO: 480 - protein, 474 aa, Curtobacterium albidum
MFSDSSEVLAFIKDTDVKFLDIRFTDLPGVQQHFNIPASTVDEDFFSVGQLFDGSSIRGFASIHESDMQLIPDVTTAYIDQFRAERTLIMIFDIYNPRNGEIYGRDPRQVAKKAEKYLASTGIADTAFFAPEAEFYIFDDVRYSVTQNKSFYSVDSEEGAWNTGREEEGGNLANKTPYKGGYFPVSPVDKTADLRDDITLKLIEAGFELERSHHEVGTGGQQEINYKFDTMVHAADDILKFKYIVKNTADQWGKVATFMPKPLFGDNGSGMHTHQSLWNDGKPLFYDENGYGGLSDLARWYIGGILKHAPALLAFTNPSINSYHRLVKGFEAPVNLVYSAGNRSAAIRIPITGTNPKAKRIEFRAPDASGNPYLAFAAQLMAGLDGIQNRIEPHEPVDKDLYELPPEEAKGIPQVPGSLDEALEALEADHEFLTKGNVFTEDLIQTWIDYKRENEILPLAQRPHPFEYELYFGV

SEQ ID NO: 481 - protein, 469 aa, Ensifer adhaerens
MTTASDILKQIKDNDVKFVDLRFTDPKGKLQHVTMDVVCVDEDMFADGVMFDGSSIGGWKAINESDMVLMPDPETAHMDPFFAQSTMVIICDILDPVSGESYNRDPRGTAKKAEAYLKASGIGDTVFVGPEAEFFVFDDVKYKADPYNTGFKLDSSELPSNDDTDYETGNLGHRPRVKGGYFPVPPIDSCQDMRSEMLTVLSEMGVTVEKHHHEVAAAQHELGVKFDALVRNADKMQIYKYVVHQVANAYGKTATFMPKPIFGDNGSGMHVHLSIWKDGKPTFAGDEYAGLSESCLYFIGGIIKHAKSLNAFTNPSTNSYKRLVPGYEAPVLLAYSARNRSASCRIPFGTNPKAKRVEVRFPDPTANPYLAFAAMLMAGLDGIKNKLHPGKAMDKDLYDLPPKELKKIPTVCGSLREALESLDKDRKYLTAGGVFDDDQIDSFIELKMQEVMRYDMTPHPVEYDMYYSV

SEQ ID NO: 482 - protein, 469 aa, Enterobacter agglomerans
MSAEHVLTMLNEHEVKFVDLRFTDTKGKEQHVTIPAHQVNAEFFEEGKMFDGSSIGGWKGINESDMVLMPDATTAVIDPFFEEPTLIIRCDILEPGTLQGYDRDPRSIAKRAEEYLRSTGIADTVLFGPEPEFFLFDDIRFGASISGSHVAIDDIEGAWNSSTKYEGGNKGHRPGVKGGYFPVPPVDSAQDIRSTMCLVMEEMGLVVEAHHHEVATAGQNEVATRFNTMTKKADEIQIYKYVVHNVAHRFGKTATFMPKPMFGDNGSGMHCHMSLSKNGTNLFSGDKYAGLSEQALYYIGGVIKHAKAINALANPTTNSYKRLVPGYEAPVMLAYSARNRSASIRIPVVASPKARRIEVRFPDPAANPYLCFAALLMAGLDGIKNKIHPGEAMDKNLYDLPPEEAKEIPQVAGSLEEALQALDADREFLTAGGVFTDDAIDAYIALRTEENDRVRMTPHPVEFELYYSV

SEQ ID NO: 483 - protein, 469 aa, Erwinia amylovora
MSAEHVLSMMNEHEVKFVDLRFTDTKGKEQHVTIPAHQVNADFFEEGKMFDGSSIGGWKGINESDMVLMPDATTAVLDPFFEDATLIIRCDILEPGTMQGYDRDPRSIAKRAEDFLRSSGIADTVLFGPEPEFFLFDDIRFGSSTSGSHVAIDDIEASWNTGKQYEGGNKGHRPGLKGGYFPVPPVDSSQDIRSAMCLTMEQMGLVVEAHHHEVATAGQNEVATRFNTMTKKADEIQIYKYVVHNVAHAYGKTATFMPKPIFGDNGSGMHCHMSLSKGGTNLFAGDKYGGLSETALFYIGGVIKHAKAINALANPTTNSYKRLVPGYEAPVMLAYSARNRSASIRIPVVASPKARRIEARFPDPAANPYLCFAALLMAGLDGIINKIHPGDAMDKNLYDLPPEEEAEIPKVAASLDEALAALNEDREFLTRGGVFTDDAIEAYIELRKAEMERVRMTPHPVEFELYYSV

SEQ ID NO: 484 - protein, 469 aa, Escherichia coli
MSAEHVLTMLNEHEVKFVDLRFTDTKGKEQHVTIPAHQVNAEFFEEGKMFDGSSIGGWKGINESDMVLMPDASTAVIDPFFADSTLIIRCDILEPGTLQGYDRDPRSIAKRAEDYLRSTGIADTVLFGPEPEFFLFDDIRFGSSISGSHVAIDDIEGAWNSSTQYEGGNKGHRPAVKGGYFPVPPVDSAQDIRSEMCLVMEQMGLVVEAHHHEVATAGQNEVATRFNTMTKKADEIQIYKYVVHNVAHRFGKTATFMPKPMFGDNGSGMHCHMSLSKNGVNLFAGDKYAGLSEQALYYIGGVIKHAKAINALANPTTNSYKRLVPGYEAPVMLAYSARNRSASIRIPVVSSPKARRIEVRFPDPAANPYLCFAALLMAGLDGIKNKIHPGEAMDKNLYDLPPEEAKEIPQVAGSLEEALNELDLDREFLKAGGVFTDEAIDAYIALRREEDDRVRMTPHPVEFELYYSV

SEQ ID NO: 485 - protein, 729 aa, Flavobacterium glycines
MSTIRFQALREASNRKVVSFEEANRKSNIFGLNVFNEKAMKQYLTSDAFKAVQGAIENGTKIERKLADYIAMGMKEWALSKGVTHYTHWFQPLTGTTAEKHDAFFETSFDGSDPVEKFGGAQLVQQEPDASSFPNGGIRNTFEARGYTAWDPTSPAFIYGTTLCIPTVFISYTGEALDNKIPLLRALAAMDEAATEVCRYFDKNVKKVTATLGWEQEYFLVDKALAHSRPDLMMTGRTLLGHTSAKGQQLDDHYFGSIPTRALTYMRDLEQECMLLGIPVKTRHNEVAPNQFELAPIFEETNLAVDHNCLLMDIMQKVAERHDFKVLLHEKPFQGVNGSGKHNNWSLATDTGVNLLSPSKTPMSNLQFLTFFINTIKAVNDYEELLRASIATASNDHRLGANEAPPAIISVFIGEQLTKVLAELESVTRGKLSPEEKTDLKLNVVGKIPEVILDNTDRNRTSPFAFTGNKFEFRAVGSSANCSNAMTTLNTIVAKQLRDFKAEVDTLIDSKAMKKDDAIFNVLREYIKVSKDILFEGDGYSEAWEKEAKKRGLSNFKTTPEALKAKVSKRALDLFSELGIMNHVEVEARYEIELEEYTKKIQIESRVLGDISKNHVIPTAIRYQNTLIENVKGLKDIFGKEFESIAKEQIGLIKEISTHIEGINAKVEEMRNERKNANALNNAQKTAEAYCNKVKPYFEIIRNHCDKLELLVDNELWTLTKYRELLFTK

SEQ ID NO: 486 - protein, 474 aa, Frankia canadensis
MFTKAEDVLRYIRDEKVQFIDVRFCDLPGIMQHFTIPTQVFAESVFTDGLMFDGSSIRGFQAIHESDMLLLPDPQTAFVDPFREHKTLAMTFFIHDPITKEQYSRDPRNIAKKAETYLRGTGIADTAYFGPEAEFYIFDDVRYDYNPYGSLHQVDSVEAAWNTARKEEGGNLGYKPRFKGGYFPVPPTDHFTDLRSEMTRVLYETGITVEMQHHEVGTAGQAEIDIRYDTLLKTADNLMLYKYVIRNVARERGKTVTFMPKPLFEDNGSGMHVHSSLWKDGEPLFYSANGYGGLSDIARYYIGGLLHHAPALLAFTNPTTNSYRRLVPGYEAPVNLVYSARNRSACCRIPLGGDSPKAKRVEFRVPDPSCNPYLAFAAMLMAGLDGIRNKIDPPEPIDKDLYELPPDELAAVPQVPGSLEKVLDALEADHEFLREGDVFTTDLIETWLDYKRRNEVDAIRLRPHPYEFTLYYDI

SEQ ID NO: 487 - protein, 486 aa, Gluconacetobacter diazotrophicus
MAKKAPSSAPTTTPSPSAEAVAKVFSLIQEHSVELVDLRFTDPRGKWHHTTQHISTIEQDTFRDGFMFDGSSIVGWKAINESDMVLLPDPTTAVMDPFSAKPQLILICDIIEPSTGQFYNRDPRATAKLAEAYLKSTGLGDTAFFGPEAEFFVFDSVKFGTGPNFGIYQLDSIEGPGASLKDYPEGNMGHRPTVKGGYFPVPPVDSENDLRAEMLSTMGEMGLPIEKHHHEVAQSQHELGTKFATLVKSADFMQIYKYCVHNVAHSYGKSATFMPKPIYGDNGSGMHVHQSIWKSGKPVFAGNGYADLSDQALYYIGGIIKHAKALNAFTNPSTNSYKRLIPGFEAPVLLAYSARNRSASCRIPYATSPKAKRVEVRFPDPTANPYLAFAAMLMAGLDGIKNKIHPGDAMDKDLYDLPPDELKQIPTVCGSLREALEALTADHEFLLAGGVFTKDQIESYIDIKWQEVFKFEHTPHPAEFEMYYSV

SEQ ID NO: 488 - protein, 460 aa, Gluconobacter oxydans
MIQEHAAEFVDLRFTDPKGKWHHTTQTVSTIEDDTFVEGFMFDGSSIQGWKAINESDMVLLPDPETAVMDPFSAKPQLILFCDIIDPKTGNFYNRDPRSTAKLAEAYLKHAGFGDTAFFGPEAEFFIFDNVQFGTGPNFGMYQLDSIEGPGASLKAYPEGNMGHRPVVKGGYFPVPPVDSENDLRAEMLSTMGEMGLPIEKHHHEVAQSQHELGTKFDTLVKSADFMQIYKYCVHNVAHSYGKTATFMPKPISGDNGSGMHVHQSIWKDGKPTFAGDKYADLSDEALYYIGGIIKHAKALNAFTNPSTNSYKRLIPGFEAPVLLAYSAANRSASCRIPHATNPKAKRVEVRFPDPTANPYLAFAAMLMAGLDGIRNKIHPGDAMDKDLYELPKEELAQIPTVCGSLREALEALKEDHAFLLEGGVFTKDQIESYIDIKWPEVYKFEHTPHPAEFEMYYSV

SEQ ID NO: 489 - protein, 471 aa, Herbaspirillum seropedicae
MARTAAEVLKMVKDNEVKFVDFRFADTRGKEQHVSVPVSHFDIDKFESGHAFDGSSIAGWKGIEASDMLLIPDPNTANIDPFMEETTLFMQCDVIEPSDGKGYDRDPRSIAKRAEAYLKSSGLGDTAYFGPEPEFFIFDGVRWGADMSGSFVKIDSEEASWSTGAKIEGGNSGHRPTVKGGYFPVPPVDSFQDMRSEMSLIIESLGIPVEVHHHEVAGAGQNELGTKFSTLVERADWTQTMKYVIWNVAHTYGKTATFMPKPLVGDNGSGMHVHQSVWKDGKNLFAGDGYAGLSEFALYYIGGIIKHAKALNAITNPGTNSYKRLVPGFEAPVKLAYSARNRSASIRIPHVANPKGRRIETRFPDPLANPYLCFSALLMAGLDGVQNKIHPGEAATKDLYHLPPEEDKLIPTVCSSLDEALEHLDKDREFLTRGGVFTDSMIDAYIDLKMQEVQRFRMTTHPIEFDMYYSL

SEQ ID NO: 490 - protein, 469 aa, Klebsiella oxytoca
MSAEHVLTMLNEHEVKFVDLRFTDTKGKEQHVTIPSHQVNAEFFEEGKMFDGSSIGGWKGINESDMVLMPDATTALIDPFYEEPTLIIRCDILEPGTLQGYDRDPRSIAKRAEEYLRSTGLADTVLFGPEPEFFLFDDIRFGASISGSHVAIDDIEGAWNSSTKYEGGNKGHRPGVKGGYFPVPPVDSSQDIRSTMCMIMEEMGLVVEAHHHEVATAGQNEIATRFNTMTKKADEIQIYKYVVHNVAHRFGKTATFMPKPMFGDNGSGMHCHMSLSKNGVNLFSGDKYAGLSEQALYYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALNALDADREFLTAGGVFTNDAIDAYIALRMEENDRIRMTPHPVEFELYYSV

SEQ ID NO: 491 - protein, 273 aa, Klebsiella variicola
CMIMEEMGLVVEAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 492 - protein, 469 aa, Kosakonia sp.
MSAEHVLTMLNEHEVKFVDLRFTDTKGKEQHVTIPAHQVNADFFEEGKMFDGSSIGGWKGINESDMVLMPDATTAVIDPFYEETTLIIRCDILEPGTMQGYDRDPRSIAKRAEEYLRSTGLADTVLFGPEPEFFLFDDVRFGSSISGSSVAIDDIEGAWNTSTKYEGGNKGHRPAVKGGYFPVPPVDSSQDLRSTMCLVMEEMGLVVEAHHHEVATAGQNEVATRFNTMTKKADEIQIYKYVVHNVAHRFGKTATFMPKPMFGDNGSGMHCHMSLSKNGTNLFSGDKYAGLSEMALYYIGGVIKHAKAINALSNPTTNSYKRLVPGYEAPVMLAYSARNRSASIRIPVVASPKARRIEVRFPDPAANPYLCFAALMMAGLDGIKNKIHPGEAMDKNLYDLPPEEAKEIPQVAGSLEEALNALDADREFLTAGGVFTDDAIDAYIALRLEENDRVRMTPHPVEFELYYSV

SEQ ID NO: 493 - protein, 445 aa, Lactobacillus helveticus
MSKQYTAEEIRKEVEDKDVRFLRLCFTDINGTEKAVEVPKSQLDKVLNNDIRFDGSSIDGFVRLEESDMVLYPDFSTWTVLPWGDEHGGKIGRLICSVHTTDGKPFAGDPRNNLKRVLGQMKDAGFDTFDIGFEMEFHLFKLDENGNWTTEVPDHASYFDMTSDDEGARCRREIVETLEGIGFEVEAAHHEVGDGQQEIDFRFDDALTTADRCQTFKMVARQIARKHGLFATFMAKPVEGQAGNGMHNNMSLFKGKHNVFYDENGEFHLSDTALYFLNGILEHARAITAIGNPTVNSYKRLIPGFEAPVYIAWAAKNRSPLVRIPSASEINTRLEMRSADPTANPYLLLAACLTAGLNGIKEQKMPMKPVEENIFEMTEEERAEHGIKPLPTTLHNAIKEFKEDDLIKSALGDHLTHSFIESKELEWSKYSQSVSDWERQRYMNW

SEQ ID NO: 494 - protein, 446 aa, Lactococcus laudensis
MTITPADILSEVKEKNVTFLRLMFTDILGTLKNVEVPATEEQLEKILDNKMMFDGSSIEGFVRINESDMYLYPDLDTWIIFPWGDENGKVAGLICDVYNPDGTPFAGDPRGNLKRALTHMETTGFKSFNLGPEPEFFLFKLDEKGNPTLEVNDQGGYFDLAPTDTADNTRREIVNVLTDLGFEVEASHHEVAVGQHEIDFKYTNVLEACDNIQIFKLVVKTIAREHGLYATFMAKPKFGINGSGMHCNMSLFDQEGNNAFYDPKGELELSETAYHFLGGILDHAYNFTAITNPTVNSYKRLVPGYEAPVYIAWAGRNRSPLVRVPASRGKGTRLELRAVDPTANPYLALAVLLECGLDGIERKLEAPAPVENNIYMMSGDERKAVGITDLPSTLHNAVKALREDDVVKAALGQHIYTNFVETKKVEWASYAQFVSQWEIDNYLYLY

SEQ ID NO: 495 - protein, 444 aa, Lysinibacillus halotolerans
MAKYTRDDIKRLVEEKEVKYIRLQFTDILGTIKNVEIPVSQLDKALDNKMMFDGSSIEGFVRIEESDMYLYPDLDTFMVFPWTAEKGKVARFICDIVNPDGTPFAGDPRSNLKRVLANMEELGFTSFNLGPEPEFFLFKLDQKGEPTLELNDDGGYFDLAPTDLGENCRRDIVLELEEMGFEIEASHHEVAPGQHEIDFKYANVVEACDNIQTFKLVVKTIARKHGLHATFMPKPLYGVNGSGMHCNVSLFRGKENAFFDEKAEIGLSETAMQFMAGVLAHVQGFTAITNPTVNSYKRLVPGYEAPCYVAWSAQNRSPLIRVPASRGLSTRIELRSVDPSANPYLALAVILESGLEGIRQKMTPPAAINRNIYIMSEEERKANGIDNLPASLSDALVALENDEVVKRALGEHIYANFREAKEIEFDMFRTAVHPWEREQYMKMY

SEQ ID NO: 496 - protein, 468 aa, Maritimibacter harenae
MSNADVLKLIKDEDIAYVDIRFTDPRGKLQHVTVMSDQVDEDFLEEGFMFDGSSVAGWKSIEASDMKLMPDSESVYIDPFYAEKTLAVHCSIVEPDTGEPYERDPRGTAEKAEAYLKSSGIGDAAFFGPEAEFFLFDDVKYSVAPNKVGYEIDADGAAWNTDTDFEMGNLGHRPDYKGGYFPVNPSDDGQDIRSEMLTTMKMIGMKVDKHHHEVATTQHELGLIFDSLTKQADELQKYKYVIHNVALSYGRSATFMPKPIKGDNGSGMHVNMSIWKDNKPLFAGDKYADLSQEALYFIGGILKHAKTLNAFTNPSTNSYKRLIPGFEAPVLRAYSARNRSGCVRIPWTESPKAKRVEARFPDPAANPYLCFAALLMAGLDGIKNKIDPGPSSDKNLYDLPPEELQGIPTVCGSLREALESLADDHDFLLAGDVFTKDQIEGYLELKWEEVYEFEHTPHPIEYKMYYSC

SEQ ID NO: 497 - protein, 344 aa, Methylobacterium aquaticum
MTKYKLEYIWLDGYTPTPNLRGKTQIKAFDSFPTLEQLPLWGFDGSSTQQAEGSSSDCVLKPVRHFPDPARTNGVLVLCEVMMPDGKTPHPSNKRATILDDEGAWFGFEQEYFFYKNGRPLGFPETGYPAPQGPYYTGVGYSNVGSVARQIVEEHLDLCLAAGINHEGINAEVAKGQWEFQIFGKGSKRAADEMWMARYLMQRLCEKYEIDIEYHCKPLGDTDWNGSGMHANFSTAYMRDVGGKAYFEKLMAAFGDAREDHIAVYGPDNHMRLTGKHETASIHTFSWGVADRGASIRVPHSFVNNDYKGYLEDRRPNSMGDPYQIASQILKTISTVPTEVSAAA

SEQ ID NO: 498 - protein, 442 aa, Paenibacillus polymyxa
MSYSREDILRIAKEENVRFIRLQFTDLLGTIKNVEIPVSQLEKALDNKMMFDGSSIEGYVRIEESDMYLYPDLDTWVVFPWVTSDRVARLICDIYKPDGVPFAGDPRGILKRVLKEAEELGYTSMNVGPEPEFFLFKTDEKGEPTTELNDQGGYFDLAPMDLGENCRREIVLKLEEMGFEIEASHHEVAPGQHEIDFKYADAIKAADQIQTFKLVVKTIARQHGLHATFMPKPLFGVNGSGMHCNQSLFKDNENVFYDETDELGLSQTARHYMAGILKHARAMAAITNPTVNSYKRLVPGYEAPCYVAWSASNRSPMIRIPASRGLSTRVEVRNPDPAANPYLALAVMLRAGLDGIKRQLALPAPIDRNIYVMSEEERIEEGIPSLPADLKEALSELIRSEVISDALGDHALAYFYELKEIEWDMYRTQVHQWERDQYLTLY

SEQ ID NO: 499 - protein, 444 aa, Priestia megaterium
MAKYTKEDIFRKVQEENVKYIRLQFTDILGTIKNVEIPVSQLEKALDNKMMFDGSSIEGFVRIEESDMYLFPDIDTFVIFPWTSEKGKVARFICDIYNADRTPFDGDPRNNLKRVLKEMEELGFTDFNLGPEPEFFLFKLDEKGEPTLELNDKGGYFDLAPTDLGENCRRDIVLELEEMGFEIEASHHEVAPGQHEIDFKYAAALKACDDIQTFKLVVKTIARKHGLHATFMPKPLFGVNGSGMHCNVSLFKDGQNAFYDENGNLELSDTARQFIAGIIKHAHSFTAVTNPTVNSYKRLVPGYEAPCYVAWSARNRSPLIRIPASRGVGTRVEVRSVDPAANPYLAMAVLLKAGLDGIKNNLEAPKPIDRNIYVMTKEERVEEGIVDLPATLAQALDSFQSNEVMVSALGEHLAEHFVEAKEIEWDMFRTQVHPWERDQYMSQY

SEQ ID NO: 500 - protein, 474 aa, Pseudarthrobacter siccitolerans
MFKTADEVLKFIKDEDIKFVDIRFTDLPGVQQHFNVPAKSVDADFFINGQLFDGSSIRGFQGIAESDMQLIPDVTTAFLDTFRMEKTLALNFSIVNPRTGDPYHRDPRGVAEKAEAYLASTGIADTAFFAPEAEFFVFDNVQYQSSPQGSFYKIDSEEAHWNTGREEEGGNLGYKTPVKGGYFPVSPTDKQADLRDAMCVALDEAGLEVERSHHEVGSAGQAEINYKFTTLTHAADDLQKFKYVIKNTADAWGKSVTFMPKPVFGDNGSGMHCHQSLWNGGEPLFYDEKGYAGLSDTARWYIGGLLKHASAVLAFTNPTVNSYRRLVKGFEAPVNMVYSQGNRSAGIRIPITGTNPKAKRIEFRAPDPSSNPYLAFAAQLMAGIDGIRNRIEPPAPIDKDLYELPAEEAKDIPKAPGTLEEALEALAEDNEFLQAGGVFTQDLIDTWIEYKYENEIRPLSLRPNPYEFELYYGV

SEQ ID NO: 501 - protein, 468 aa, Pseudomonas azotoformans
MSKSVQLIKDHDVKWIDLRFTDTKGTQHHVTMPARDALDEAFFEEGKMFDGSSIAGWKGIEASDMILMPDDSTAVLDPFTEEPTLILVCDVIEPSTMQGYDRDPRAIAKRAEEYLKSTGIGDTVFVGPEPEFFIFDQVKFKSDISGSMFKIYSEQGSWMSDQDVEGGNHGHRPGIKGGYFPVPPFDHDHEIRTSMCNAMEDMGLVIEVHHHEVATAGQNEIGVKFNTLVAKADEVQTLKYCVHNTAVAYGRTATFMPKPLYGDNGSGMHVHLSIAKDGKNTFAGEGYAGLSDTALYFIGGIIKHGKALNGFTNPSTNSYKRLVPGFEAPVMLAYSARNRSASIRIPYVSSPRARRIEARFPDPAANPYLAFAALVMAGLDGIQNKIHPGDAADKNLYDLPPEEAKEIPQVCGSLKEALEELDKGRAFLTKGGVFSDDFIDAYIGLKSEEEIKVRTFVHPLEYELYYSC

SEQ ID NO: 502 - protein, 468 aa, Pseudomonas putida
MSKSVQLIKDHDVKWIDLRFTDTKGTQHHVTMPARDALEDDFFEVGKMFDGSSIAGWKGIEASDMILMPVDETAVLDPFTEEPTLIITCDIVDPSSMQGYDRDPRAIAKRAEEYLKSTGIGDTVFAGPEPEFFIFDEVKFQSDISGSMFKIFSEQGSWMTGADVEGGNKGHRPGVKGGYFPVPPFDHDHEIRTAMCNALEEMGQTVEVHHHEVATAGQNEIGVKFNTLVKKADEVQALKYVVHNVADAYGRTATFMPKPLYGDNGSGMHVHMSIWKDGKNTFSGEGYAGLSDTALYFIGGIIKHGKALNGFTNPSTNSYKRLVPGFEAPVMLAYSARNRSASIRIPYVGSPKARRIEARFPDPSANPYLAFAALLMAGLDGIQNKIHPGDAADKNLYDLPPEEAKDIPQVCGSLKEALEELDKGRAFLTKGGVFSDDFIDAFIELKSEEEIKVRTFVHPLEYELYYSC

SEQ ID NO: 503 - protein, 468 aa, Pseudomonas syringae
MSKSVQLIKDHDVKWIDLRFTDTKGTQHHVTMPARDALDEDFFEIGKMFDGSSIAGWKGIEASDMILMPDDSTAVLDPFTEEPTIILVCDIIEPSTMQGYDRDPRAIAHRAEEYLKSTGIGDTVFVGPEPEFFIFDEVKFKSDISGSMFKIFSEQGSWMSDQDVEGGNKGHRPGVKGGYFPVPPFDHDHEIRTSMCNAMEEMGLVIEVHHHEVATAGQNEIGVKFNTLVKKADEVQTLKYCVHNVADAYGRTATFMPKPLYGDNGSGMHVHMSISKDGKNTFAGEGYAGLSDTALFFIGGIIKHGKALNGFTNPSTNSYKRLVPGFEAPVMLAYSARNRSASIRIPYVSSPKARRIEARFPDPAANPYLGFAALLMAGLDGIQNKIHPGDAADKNLYDLPPEEAKEIPQVCGSLKEALEELDKGRAFLTKGGVFSDDFIDAYIALKSEEEIKVRTFVHPLEYELYYSC

SEQ ID NO: 504 - protein, 469 aa, Rahnella contaminans
MSAEHVLTMLNEHEVKFVDLRFTDTKGKEQHVTIPAHQVSADFFEEGKMFDGSSIGGWKGINESDMVLMPDASTAVLDPFYEETTLIIRCDILEPTTLQGYDRDPRSTAKRAEDFLRSSGIADTVLFGPEPEFFLFDDVRFGSSISGSHVAIDDIEGAWNSSTKYEGGNKGHRPAVKGGYFPVPPVDSSQDLRSTMCLTMEEMGLVVEAHHHEVATAGQNEVATRFNTLTKKADEIQIYKYVVHNVAHAFGKTATFMPKPMFGDNGSGMHCHMSLSKNGNNLFAGDKYGGLSEMALFYIGGIIKHAKAINAYANPTTNSYKRLVPGYEAPVMLAYSARNRSASIRIPHVSSPKAMRIEARFPDPAANPYLAFAALMMAGLDGIINKIHPGDAMDKNLYDLPPEEEAEIPKVAGSLEEALNALNEDREFLTRGGVFTDDAIDAYIGLRKEEMDRVRMTPHPVEFELYYSV

SEQ ID NO: 505 - protein, 469 aa, Rahnella sp.
MSAEHVLTMLNEHEVKFVDLRFTDTKGKEQHVTIPAHQVSADFFEEGKMFDGSSIGGWKGINESDMVLMPDASTAVLDPFYEETTLIIRCDILEPTTLQGYDRDPRSTAKRAEDFLRSSGIADTVLFGPEPEFFLFDDVRFGSSISGSHVAIDDIEGAWNSSTKYEGGNKGHRPAVKGGYFPVPPVDSSQDLRSTMCLTMEEMGLVVEAHHHEVATAGQNEVATRFNTLTKKADEIQIYKYVVHNVAHAFGKTATFMPKPMFGDNGSGMHCHMSLSKNGNNLFSGDKYGGLSEMALFYIGGIIKHAKAINAYANPTTNSYKRLVPGYEAPVMLAYSARNRSASIRIPHVSSPKAMRIEARFPDPAANPYLAFAALMMAGLDGIINKIHPGDAMDKNLYDLPPEEEAEIPKVAGSLEEALNALNEDREFLTRGGVFTDDAIDAYIGLRKEEMDRVRMTPHPVEFELYYSV

SEQ ID NO: 506 - protein, 469 aa, Rhizobium leguminosarum
MATASEILKQIKENDVKFVDLRFTDPKGKLQHVTMDVVCVDEDMFADGVMFDGSSIGGWKAINESDMVLMPDTETVHMDPFFAQSTMVIVCDILDPVSGEAYNRDPRGTAKKAEAYLKASGIGDTIFVGPEAEFFVFDDVKYKADPYNTGFKLDSTELPSNDDTDYETGNLGHRPRVKGGYFPVPPVDSAQDMRSEMLTVLSEMGVVVEKHHHEVAAAQHELGIKFDTLVRNADKMQIYKYVVHQVANAYGKTATFMPKPIFGDNGSGMHVHQSIWKGGKPTFAGDEYAGLSESCLFYIGGIIKHAKAINAFTNPSTNSYKRLVPGYEAPVLLAYSARNRSASCRIPFGSNPKAKRVEVRFPDPTANPYLAFAAMLMAGLDGIKNKIHPGKAMDKDLYDLPPKELKKIPTVCGSLREALESLDKDRKFLTAGGVFDDDQIDAFIELKMAEVMRFEMTPHPVEYDMYYSA

SEQ ID NO: 507 - protein, 478 aa, Rhodococcus oryzae
MAFTTAEEVINYIADEKIEYVDIRFSDLPGVQQHFSIPASAFNKDVFEDGLAFDGSSVRGFQSIHESDMMLLPDVSTARVDPFRAAKTLNMDFFVHDPFTRESYSRDPRNVARKAEEYLVSTGIADTAFFGAEAEFYIFDSVRYDSGMNGAFYELDSISGSWNTGNEVNADGSPNLGYKVRPKGGYFPVAPYDHYVDLRDEISTNLQNAGFELERGHHEVGTGGQQEINYKFNTLLAAADDLQLFKYIVKNTCWQNGKSATFMPKPLFGDNGSGMHVHQSLWKDGKPLFHDESGYAGLSDMARHYIGGILHHAPSLLAFTNPTVNSYHRLVPGYEAPINLVYSQRNRSAAVRIPITGNNPKAKRIEFRAPDSSGNPYLNFAAQMMAGLDGIKNKIEPMAPVDKDLYELPPEEARNIPQAPTSLATVIDRLEADHEYLTEGGVFTTDLIESWISIKREQEIAPVNLRPHPYEFQLYYDV

SEQ ID NO: 508 - protein, 473 aa, Sphingomonas glacialis
MAKAPNTASDVLKMIKEQEIEWVDLRFTDPKGKWQHLTMVASVVGEDELTDGFMFDGSSIEGWKAINESDMILMPDLDAIWTDPFSATPMLILVCDIVEPSTGELYARDPRSTAKRAEAYLKTTGIGDTIFIGPEAEFFMFDNVQFENSYSTSYYKIDDIELPTNTGTSYDGGNLGHRPRAKGGYFPVAPVDSAVDIRGEMVSTMLEMGLPCDKHHHEVAAAQHELGLTFGTMTVTADRMQIYKYVCHNVAQAYGKTVTFMPKPIKEDNGSGMHTHLSIWDKGNPLFAGNGYAGLSDMCLYFIGGIIKHAKAVNAFTNPTTNSYKRLVPGYEAPVLLAYSARNRSASCRIPYGTGPKAKRVEVRFPDAMANPYLCYAALFMAGLDGIQNRIHPGEAMDKNLYDLPPEELAEVPTVCGSLREALDCLAADHDFLLKGDVFSKDQIESYIELKMEDVKRWEMTPSPVEFDMYYSY

SEQ ID NO: 509 - protein, 469 aa, Stenotrophomonas maltophilia
MSVENVEKLVKDNQIEFVDLRFVDMRGIEQHVTFPVSIIEASLFEEGKMFDGSSIAGWKGINESDMVLLPDPSSAYVDPFYADPTIVISCDILDPATMQPYGRCPRGIAKRAEAFLKSSGIAEQAFFGPEPEFFIFDSVRFANDMGNTFFKVDSEEAAWNTGAKYDGANSGYRPGVKGGYFPVPPTDSLHDLRAEMCKTLEQVGIEVEVQHHEVATAGQCEIGTKFSTLVQKADELLRMKYVIKNVAHRNGKTATFMPKPIVGDNGSGMHVHQSLSKGGTNLFSGDGYGGLSQLALWYIGGIFKHAKAINAFANAGTNSYKRLVPGFEAPVMLAYSARNRSASCRIPFVTNPKARRIEMRFPDPIQSGYLTFTVLMMAGLDGIRNQIDPGAPSDKDLYDLPPEEEKLIPQVCSSLDQALEALDKDREFLKAGGVMDDDFIDGYIALKMQEVTKFRAATHPLEYQLYYAS

SEQ ID NO: 510 - protein, 468 aa, Stutzerimonas stutzeri
MSKSLQLIKDYDVKWIDLRFTDTKGKQHHVTVPARDAQDEDFFEHGKMFDGSSIHGWKGIEASDMILMPVDETAVLDPFTEEPTLILVCDIVEPSTMQGYDRDPRSIAKRAEEFLKGTGIGDTVFVGPEPEFFIFDQVKFKSDISGSMFKIYSEQGSWMTDQDVEGGNKGHRPAVKGGYFPVPPCDHDHEIRTAMCNAMEEMGLVVEVHHHEVATAGQNEIGVKFNTLVAKADEVQTLKYCVHNVADAYGKTATFMPKPLYGDNGSGMHVHMSISKDGKNTFAGEGYAGLSETALYFIGGIIKHGKALNGFTNPSTNSYKRLVPGFEAPVMLAYSARNRSASIRIPYVSSPKARRIEARFPDPAANPYLCFAALLMAGLDGIQNKIHPGDAADKNLYDLPPEEGKLIPQVCGSLKEALEELDKGRAFLTKGGVFSDEFIDAYIELKSEEEIKVRTFVHPLEYDLYYSV

SEQ ID NO: 511 - protein, 471 aa, Variovorax paradoxus
MAKTVADVLKLVKENEVKFIDFRFTDTRGKEQHVTVPVSAFDEDKFTSGHAFDGSSIAGWKGIEASDMQLMPDPNTANIDPFFEETTLILTCDVIDPADGKAYERDPRSLAKRAEAYMKASGLGDTAFFGPEPEFFVFDGVRWKNDMSGCFVKIDSEEASWNTDKEYEHGNTGHRPAVKGGYFPVPPVDSFQDMRSEMCLVLESLGIPVEVHHHEVANAGQMELGTKFSTLVQRADWVQLQKYVIHNVAHAYGKTATFMPKPIVGDNGSGMHVHQSVWKDGKNLFAGDGYAGLSDFALHYIGGIIKHARALNAITNPGTNSYKRLVPGFEAPVKLAYSAKNRSASIRIPFVANPKGRRVEARFPDPLMNPYLGFAALLMAGLDGVENKIHPGEAASKDLYHLPPEEDALIPTVCHSLDQALEYLDKDRAFLTKGGVFTDAYIDAYIELKMQEVTRFRMATHPVEFDMYYSL

SEQ ID NO: 512 - protein, 468 aa, Yoonia vestfoldensis
MDKKAVLKLIKDEEVDYVDIRFTDPRGKLQHVTVVADLVDEDFLDEGFMFDGSSIAGWKSIDQSDMKLMPDASSAYVDPFYAEKTICIHCNVVEPDTMEPYDRDPRGTAVKAEEYLKSSGIGDSAFFGPEAEFFVFDDVRFSVSMNKVSYQVDALDGAWNTDTEYEMGNTGHRPGIKGGYFPVNPIDDAQEMRSEMLSTMKRMGMKVDKHHHEVASCQHELGLIFGTLTHQADEIQKYKYVIHNVAQAYGKSATFMPKPIAGDNGTGMHVNMSIWKDGKPLFAGDKYADLSQEALYFIGGILKHAKALNAITNPTTNSYKRLIPGFEAPVLRAYSARNRSGCVRIPWAESPKAKRVEARFPDPAANPYLAFAALLMAGLDGIKNKIDPGPSSDKDLYDLPPEELASIPTVCGSLREALESLEADHEFLLAGDVFTKSQLEGYMALKWEEVYAYEHTPHPIEFKMYYSC

SEQ ID NO: 513 - protein, 346 aa, Rahnella aceris
MKLMRNTVAGLVAATFSLTAMSAFAAANLTGAGGTFPAPVYAKWADAYQKATGTQVNYQGIGSSGGVKQIIAKTVDFGASDAPMKEEDLNKNGLFQFPTVIGGVVLAVNIPGIKSGQLTLDGATLGDIYLGKIKKWNDTAITKLNPGVKLPDTNIAVVRRADGSGTSFVFTSYLAKVNSEWKDKIGAGSTVNWPTGLGGKGNDGVAAFVQRLPGSIGYVEYAYAKQNNLAYTKLVDADGKAISPTEESFSAAAKGADWSKTFAQDLTNQKGDNAWPISSTTFILIYKDQQDAAKGTEVLKFFDWAYKNGNKLTTDLDYAALPASVVEQIRAAWKTNIKDSSGKALY

SEQ ID NO: 514 - protein, 53 aa, synthetic construct
  VARIANT 8: note = X is T or V
  VARIANT 18: note = X is V or I
  VARIANT 43: note = X is A or T
  VARIANT 44: note = X is Y or W
  VARIANT 48: note = X is V or F
  VARIANT 49: note = X is D or S
GSTVNWPXGLGGKGNDGXAAFVQRLPGSIGYVEYAYAKQNNLXXTKLXXADGK

SEQ ID NO: 515 - DNA, 458 bp, Klebsiella variicola
  source 1..458: mol_type = genomic DNA
gacgatcgcccctatcttctatacggtaccgctgcagctgttggcttatcacgtcgcgctgatcaaaggcactgacgttgaccagccgcgtaacctggcgaaatcggtaacggttgagtaaaaccatcattacttgcaaagggctgccggcggcggccctttttattttcacctgctgtttattaccgaacttgcgcgaaatattttggctggcggtaggtgacaaacgaacgttttgcggtccgatttcgctgtcataaaaagaaaattattctgtcattgcacggtcatgattacagataactatctgagttatatatttattatttgctgtaaaatcccttctttctgcctgtcataaaactgtcataattcgtacattttactgtcacctctttgacctattttgctcaccatagcctcgaaacaatgatttacgaatcccagcaggagacatt

SEQ ID NO: 516 - protein, 207 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 517 - DNA, 460 bp, Kosakonia sacchari
  source 1..460: mol_type = genomic DNA
caggatggcttctcattgcgccgttttccgtactgtctctgattttttgggggattgggatggtgctgttatggctggcgcgacaagtgatgaatggaccggaacttacgaaattagtgcagtggcgcgtatcgctaaaaatcgcccaattccggcgccaggcgctgacacaatctaagcgtaaataaatcgataagcggtggctttttctcgcgttttgttaggacttcttgtcagattcagctacgtttttaatgtcataaaactaaaatcttgctgacatcttatcgtcatcaacctttttatctcgctgataaatcatccaggattcgttaaatctggccgcaattcgttgctgtcataaaacagtaacaatctttgcatataactgtcatcaaagtgtcctattttgctcatcgtagccactaaacaacgatttacgattcttgcaggagacatt

SEQ ID NO: 518 - DNA, 345 bp, Rahnella aceris
  source 1..345: mol_type = genomic DNA
gtgccatcaaaacctgctcaggctaaaaaaccggctgtcactaaaacgaaaaacagtgaaaaacctgaagtgaattccgcatcccgcaagcgttttgcccagcctggccgtaagaagaaggggcgttaatttacctgcttttcattatccgccggtgtaataaccggctttttctgcttaaaactcccgcattttgcccgtatggttcgaattgtgaccagtgtcataaaactgtcatataacgtacattttactgtcactaaactgtcctattttcctcctgtgccaaacactttaattaacgtcgacatcagtcgggaagtttaaaaccaccaggagtggaac

SEQ ID NO: 519 - protein, 469 aa, Kosakonia sacchari
MSAEHVLTMLNEHEVKFVDLRFTDTKGKEQHVTIPAHQVNADFFEEGKMFDGSSIGGWKGINESDMVLMPDATTAVIDPFYEESTLIIRCDILEPGTMQGYDRDPRSIAKRAEEYLRSTGLADTVLFGPEPEFFLFDDVRFGSSISGSSVAIDDIEGAWNTSTKYEGGNKGHRPAVKGGYFPVPPVDSSQDLRSTMCLVMEEMGLVVEAHHHEVATAGQNEVATRFNTMTKKADEIQIYKYVVHNVAHRFGKTATFMPKPMFGDNGSGMHCHMSLSKNGTNLFSGDKYAGLSEMALYYIGGVIKHAKAINALSNPTTNSYKRLVPGYEAPVMLAYSARNRSASIRIPVVASPKARRIEVRFPDPAANPYLCFAALLMAGLDGIKNKIHPGEAMDKNLYDLPAEEAKEIPQVAGSLEEALNALDADREFLTAGGVFTDDAIDAYIALRIEENDRVRMTPHPVEFELYYSV

SEQ ID NO: 520 - protein, 469 aa, Klebsiella variicola
MSAEHVLTMLNEHEVKFVDLRFTDTKGKEQHVTIPSHQVNAEFFEEGKMFDGSSIGGWKGINESDMVLMPDASTAVIDPFYEEPTLIIRCDILEPGTLQGYDRDPRSIAKRAEEYLRATGIADTVLFGPEPEFFLFDDIRFGASISGSHVAIDDIEGAWNSSTKYEGGNKGHRPGVKGGYFPVPPVDSSQDIRSTMCMIMEEMGLVVEAHHHEVATAGQNEVATRFNTMTKKADEIQIYKYVVHNVAHRFGKTATFMPKPMFGDNGSGMHCHMSLAKNGTNLFSGDKYAGLSEQALFYIGGVIKHAKAINALANPTTNSYKRLVPGYEAPVMLAYSARNRSASIRIPVVTSPKARRIEVRFPDPAANPYLCFAALLMAGLDGIKNKIHPGEAMDKNLYDLPPEEAKEIPQVAGSLEEALQALDADREFLTAGGVFTNDAIDAYIALRLEENDRVRMTPHPVEFELYYSV

SEQ ID NO: 521 - protein, 469 aa, Rahnella aceris
MSAEHVLTMLNEHEVKFVDLRFTDTKGKEQHVTIPAHQVSADFFEEGKMFDGSSIGGWKGINESDMVLMPDASTAVLDPFYEETTLIIRCDILEPTTLQGYDRDPRSTAKRAEDFLRSSGIADTVLFGPEPEFFLFDDVRFGSSISGSHVAIDDIEGAWNSSTKYEGGNKGHRPAVKGGYFPVPPVDSSQDLRSTMCLTMEEMGLVVEAHHHEVATAGQNEVATRFNTLTKKADEIQIYKYVVHNVAHAFGKTATFMPKPMFGDNGSGMHCHMSLSKNGNNLFAGDKYGGLSEMALFYIGGIIKHAKAINAYANPTTNSYKRLVPGYEAPVMLAYSARNRSASIRIPHVSTPKAMRIEARFPDPAANPYLAFAALMMAGLDGIINKIHPGDAMDKNLYDLPPEEEAEIPKVAGSLEEALNALNEDREFLTRGGVFTDDAIDAYIGLRKEEMDRVRMTPHPVEFELYYSV

SEQ ID NO: 522 - DNA, 1653 bp, synthetic construct
  source 1..1653: mol_type = other DNA
ggacatgcgatcgtattgcctatgaggaccaaaacgaaaaaaggcccccctttcgggaggcctcttttctggaatttggtaccgagctatctaaggatggagggccaaggagcgcaataaaaaagcccccggaaggtgatcttccgggggctttctcatgcgtttcggcccgggcaacatcagatgccgacatattcgggagttccctcatttaggaccctaccaggtctcagtcaacattgcgacgatcgcccctatcttctatacggtaccgctgcagctgttggcttatcacgtcgcgctgatcaaaggcactgacgttgaccagccgcgtaacctggcgaaatcggtaacggttgagtaaaaccatcattacttgcaaagggctgccggcggcggccctttttattttcacctgctgtttattaccgaacttgcgcgaaatattttggctggcggtaggtgacaaacgaacgttttgcggtccgatttcgctgtcataaaaagaaaattattctgtcattgcacggtcatgattacagataactatctgagttatatatttattatttgctgtaaaatcccttctttctgcctgtcataaaactgtcataattcgtacattttactgtcacctctttgacctattttgctcaccatagcctcgaaacaatgatttacgaatcccagcaggagacattgcggtatatgaatgcgacctcgggcccaagttcacttaaaaaggagatcaacaatgaaagcaattttcgtactgaaacatcttaatcatgctaaggaggttttctaatgagcaagggcgaggaactctttaccggagtcgtgccgattttggtggaactggacggtgacgtgaatgggcataaattcagtgtacggggtgagggcgaaggcgatgcaaccaacggcaagctgacacttaaatttatctgcacgaccggcaaactaccggttccttggccgacgctggttacgacgctgacctacggtgttcagtgtttttcccgttacccggatcatatgaaacgtcacgatttctttaaatcggcaatgccagaaggatatgtccaagagcgaaccattagctttaaagacgatgggacctataaaacgcgcgcggaggttaaattcgagggcgacactttggtcaaccgtatcgaactgaagggtatcgactttaaggaagatggtaatattttaggtcataaactggaatacaacttcaacagtcataatgtctatataactgccgataagcaaaaaaacggcattaaagcgaatttcaagattcgccataatgtggaagatggatccgttcagttggcggatcactatcagcagaacactcccatcggcgatgggccagtactgttacctgacaatcactatctgtctacccagtcagtgctgagcaaagatccgaacgaaaaacgcgatcacatggtactgctcgaattcgtgacagcggctggcattacacatggtatggatgaactttacaaataaccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 523 - protein, 346 aa, Klebsiella variicola
MNVMRTTVATVVAATLSMSAFSAFAAASLTGAGATFPAPVYAKWADTYQKETGNKVNYQGIGSSGGVKQIIANTVDFGASDAPLADDKLTQEGLFQFPTVIGGVVLAVNLPGVKSGELVLDGKTLGDIYLGKIKKWDDAAIAKLNPGLKLPSQNIAVVRRADGSGTSFVFTSYLSKVNEEWKSKIGAGSTVNWPTGLGGKGNDGIAAFVQRLPGSIGYVEYAYAKQNNLAYTKLVSADGKPVSPTEDNFANAAKGVDWSKSFAQDLTNQKGENAWPITSTTFILVHKATNKPEQTAEVLKFFDWAYKNGGKEANALDYATLPESVVEQVRAAWKTNVKDSSGKALY

SEQ ID NO: 524 - protein, 345 aa, Kosakonia sacchari
MAMLNASVRLMAAALALSTTTIFAATNVTGAGGTFPAPVYAKWAAEYQKQTGSQVNYQGIGSSGGIKQIIAKTVDFGASDAPMSDDDLAKNGLFQFPTVIGGVVLAVNIPGVKSGELTLDGKTLGDIYLGTVKKWNDPEITKLNPKVKLPDTNINVVRRADGSGTSFVFTSYLSKVNADWASKVGKGSTVNWPVGLGGKGNDGVAAFVQRLPGSIGYVEYAYAKQNNLTWTKLFDADGKVVEPSQQSFSNSAKGADWSKSFAQDLTFQKGKEAWPISSTTFILVYKKQDNAAKGAEVLKFFDWSYKNGSTIATDLDYAPLPDSVTAQVRAAWKANVQDASGKPLF

SEQ ID NO: 525 - DNA, 17 bp, Klebsiella variicola
  source 1..17: mol_type = genomic DNA
tgtcataaaactgtcat

SEQ ID NO: 526 - DNA, 17 bp, Klebsiella variicola
  source 1..17: mol_type = genomic DNA
tgtcataattcgtacat

SEQ ID NO: 527 - DNA, 17 bp, Klebsiella variicola
  source 1..17: mol_type = genomic DNA
tgtcacctctttgacct

SEQ ID NO: 528 - DNA, 17 bp, Klebsiella variicola
  source 1..17: mol_type = genomic DNA
tccgatttcgctgtcat

SEQ ID NO: 529 - DNA, 17 bp, Klebsiella variicola
  source 1..17: mol_type = genomic DNA
tgtcattgcacggtcat

SEQ ID NO: 530 - protein, 474 aa, Paenibacillus graminis
MSVEKVLQTIKENNIEWVDFRFVDLGGRAHHISLPASAVDDETFVNGVAFDGSSIAGFRGIEESDMVMMPDPSTIYIDPFTAHPTLNIMCDIFTPDGERYERDPRGIAVKAEEFLQASGVGTAAFFAPESEFFIFDDVRYESGMNSSSYFVDSEEAVWNTNRKDEGGNMAFKVGVKGGYVPVAPVDSQQDIRSEMCRLLSEAGLEIERHHHEVATAGQAEINFRFDTLKKTADNLLTYKYIVQNTARQYGKVATFMPKPLFGDNGSGMHVHQSIFNGDSPLFYEKGAYANLSEMALHYIGGILYHAPALIALTNPSTNSFKRLVPGYEAPVNLVFSKGNRSAAVRIPVAAVTPKGCRIEFRTPDSTANPYLAFSAMLMAGLDGIKKKINPVELGYGPLDKNIYELADEDKEKIRSVPGTLEEALDSLEADYEFLTEGGVFTKDFIDNYIALKRSEAQAVAIRVHPHEYSLYFDV

SEQ ID NO: 531 - protein, 469 aa, Enterobacter sp
MSAEHVLTMLNEHEVKFVDLRFTDTKGKEQHVTIPAHQVNADFFEEGKMFDGSSIGGWKGINESDMVLMPDATTAVIDPFYEEPTLIIRCDILEPGTMQGYDRDPRSIAKRAEEYLRATGIADTVLFGPEPEFFLFDDVRFGSSISGSHVAIDDIEGAWNSATKYEGGNKGHRPAVKGGYFPVPPVDSAQDLRSTMCLVMEEMGLVVEAHHHEVATAGQNEVATRFNTMTKKADEIQIYKYVVHNVAHRFGKTATFMPKPMFGDNGSGMHCHMSLSKNGTNLFSGDKYAGLSEQALFYIGGVIKHAKAINALANPTTNSYKRLVPGYEAPVMLAYSARNRSASIRIPVVASPKARRIEVRFPDPAANPYLCFAALLMAGLDGIKNKIHPGEAMDKNLYDLPPEEAAEIPQVAGSLEEALNALDADREFLTAGGVFTDDAIDAYIALRVEENDRVRMTPHPVEFELYYSV

SEQ ID NO: 532 - protein, 469 aa, Phytobacter diazotrophicus
MSAEHVLTMLNEHEVKFVDLRFTDTKGKEQHVTIPAHQVNADFFEEGKMFDGSSIGGWKGINESDMVLMPDASTAVIDPFYEEPTLIIRCDILEPGTMQGYDRDPRSIAKRAEEYLRATGIADTVLFGPEPEFFLFDDVRFGSSISGSHVAIDDIEGAWNSSTKYEGGNKGHRPAVKGGYFPVPPVDSAQDLRSTMCLVMEEMGLVVEAHHHEVATAGQNEVATRFNTMTKKADEIQIYKYVVHNVAHRFGKTATFMPKPMFGDNGSGMHCHMSLSKNGVNLFSGDKYAGLSEQALYYIGGVIKHAKAINALSNPTTNSYKRLVPGYEAPVMLAYSARNRSASIRIPVVASPKARRIEVRFPDPAANPYLCFAALLMAGLDGIKNKIHPGEAMDKNLYDLPAEEAKEIPQVAGSLEEALNCLNEDREFLTAGGVFTDDAIDAYIALRIEENDRVRMTPHPVEFELYYSV

SEQ ID NO: 533 - protein, 469 aa, Enterobacter dykesii
MSAEHVLTMLNEHEVKFVDLRFTDTKGKEQHVTIPAHQVNAEFFEEGKMFDGSSIGGWKGINESDMVLMPDATTAVIDPFFEEPTLIIRCDILEPGTLQGYDRDPRSIAKRAEEYLRSTGIADTVLFGPEPEFFLFDDIRFGASISGSHVAIDDIEGAWNSSTKYEGGNKGHRPGVKGGYFPVPPVDSAQDIRSTMCLVMEEMGLVVEAHHHEVATAGQNEVATRFNTMTKKADEIQIYKYVVHNVAHRFGKTATFMPKPMFGDNGSGMHCHMSLSKNGTNLFSGDKYAGLSEQALYYIGGVIKHAKAINALANPTTNSYKRLVPGYEAPVMLAYSARNRSASIRIPVVASPKARRIEVRFPDPAANPYLCFAALLMAGLDGIKNKIHPGEAMDKNLYDLPPEEAKEIPQVAGSLEEALQALDADREFLTAGGVFTDDAIDAYIALRMEENDRVRMTPHPVEFELYYSV

(19) United States
(12) Patent Application Publication
(10) Pub. No.: US 2025/0243130 A1
(43) Pub. Date: Jul. 31, 2025

(54) PHOSPHATE SENSING MICROBIAL GENE SWITCH

(71) Applicant: SWITCH BIOWORKS, INC., San Carlos, CA (US)

(72) Inventors: Caroline Kukolj, Redwood City, CA (US); Tim Schnabel, Menlo Park, CA (US); Timothy Lyndon Haskett, Menlo Park, CA (US); Gregory Alexander Knauf, San Mateo, CA (US); Tomasz Zajkowski, Warsaw (PL); Elizabeth Leigh Ordeman, Palo Alto, CA (US); Niklaus Hoyt Evitt, New Haven, CT (US); James Pearce, La Jolla, CA (US); Sandipan Samaddar, Redwood City, CA (US); Daniel Torres Naylor, San Mateo, CA (US); Hsiao-Han Lin, San Carlos, CA (US); Alexis Makena Chun, San Bruno, CA (US)

(21) Appl. No.: 19/042,264

(22) Filed: Jan. 31, 2025

Related U.S. Application Data
(60) Provisional application No. 63/627,673, filed on Jan. 31, 2024.

(51) Int. Cl. C05F 11/08; A01C 21/00; C05G 1/00; C05G 3/60; C12N 9/00; C12N 15/74

(52) U.S. Cl. CPC C05F 11/08; A01C 21/007; C05G 1/00; C05G 3/60; C12N 9/93; C12N 15/74; C12Y 603/01002; C12N 2830/003; C12N 2830/005; C12N 2830/48

(57) ABSTRACT

Genetically engineered bacteria which express RNAs or proteins that produce ammonia upon decreases in phosphate concentrations are disclosed.

### CROSS-REFERENCE TO RELATED APPLICATIONS

This non-provisional US patent application claims benefit of U.S. Provisional Patent Application Ser. No. 63/627,673, filed Jan. 31, 2024, which is incorporated herein by reference in its entirety.

### STATEMENT REGARDING FEDERALLY SPONSORED RESEARCH

Not applicable.

### INCORPORATION OF SEQUENCE LISTINGS

A sequence listing contained in the xml file named “P14613US02.xml” which is 686,780 bytes in size (measured in MS-Windows), which was created on Jan. 28, 2025, and which comprises 533 sequences, is electronically filed herewith and is incorporated herein by reference in its entirety. The sequence listing contained in the xml file named “P14613US00.xml” which is 658,989 bytes in size (measured in MS-Windows), which was created on Jan. 31, 2024, which comprises 512 sequences, and which was filed in U.S. Provisional Patent Application Ser. No. 63/627,673 on Jan. 31, 2024, is also incorporated herein by reference in its entirety.

### BACKGROUND

The expression of certain proteins or production of certain metabolites can be inhibitory to a host strain and reduce its growth rate. Under lab conditions this issue can be overcome by using inducible expression systems with chemical inducers such as tetracycline. However, for bacteria in their natural environments (e.g., soil-borne bacteria in soil) such systems are not suitable either because these inducers are not compatible with environmental requirements and laws, are very expensive, or can simply not reach the host (e.g., in soil). For example, bacteria that are producing and releasing large quantities of ammonia which can be used by crop plants suffer a marked fitness defect that would render them non-competitive and unable to persist in the environment. Therefore, there is a need to provide alternative solutions which allow expression of proteins or production of certain metabolites in bacteria in their natural environments after a desirable delay and/or under certain conditions.

### SUMMARY

Methods of providing at least one agriculturally relevant compound to a plant comprising placing at least one genetically engineered bacterium into a plant growth medium, wherein the genetically engineered bacterium comprises a heterologous gene expression cassette comprising at least one nucleic acid sequence coding for at least one RNA sequence or protein of interest, wherein the heterologous gene expression cassette is operably linked to a control element comprising a phosphate-sensitive promoter, wherein said at least one RNA sequence or protein of interest is or causes the production of said at least one agriculturally relevant compound when a decrease in phosphate concentration in the plant growth medium activates expression of said at least one RNA sequence or protein of interest are provided.

Genetically engineered bacterium comprising a heterologous gene expression cassette comprising at least one nucleic acid sequence coding for at least one RNA sequence or protein of interest operably linked to a control element comprising a phosphate-sensitive promoter, wherein said at least one RNA sequence or protein of interest is or causes the production of at least one agriculturally relevant compound are provided.

Compositions comprising the genetically engineered bacterium and an agriculturally acceptable carrier are also provided.

Plant parts or plant propagules which are at least partially coated, imbibed, or mixed with the compositions are also provided. Use of the plant parts or plant propagules which are at least partially coated, imbibed, or mixed with the compositions to grow a crop are also provided.

Agricultural systems comprising: (i) at least one of the engineered bacteria; (ii) at least one plant growth medium; and (iii) at least one crop plant, crop plant seed, or crop plant vegetative propagule; wherein the plant growth medium, crop plant, crop seed, and/or crop plant propagule comprise, are at least partially coated, imbibed, and/or are mixed with the engineered bacterium or a composition comprising the engineered bacterium and an agriculturally acceptable carrier, are provided.

Treated plant seed or plant propagule systems comprising: (i) at least one crop plant seed or crop plant vegetative propagule; and (ii) at least one of the engineered bacteria, wherein the crop plant seed or crop plant propagule are at least partially coated, imbibed, and/or mixed with the engineered bacterium or a composition comprising the engineered bacterium and an agriculturally acceptable carrier, are provided.

### BRIEF DESCRIPTION OF THE DRAWINGS

FIG. 1 shows a genetic reporter circuit for measuring expression of a green fluorescent protein reporter under control of the phosphate-sensitive pstS and Pliar53 promoters when there is a decrease in phosphate concentration in a bacterial growth medium.

FIG. 2 shows low-Pi dependent induction of expression (defined as GFP/constitutive LSS mScarlett fluorescence) from the pstS reporters in model bacterial strains incubated for 15 h in minimal media with or without supplementation of 500 uM potassium phosphate. Error bars represent one standard deviation from mean calculated from 3 biological replicates.

FIG. 3 shows low-Pi dependent induction of expression (defined as GFP/constitutive LSS mScarlett fluorescence) from the Pliar53 reporters in model bacterial strains incubated for 15 h in minimal media with or without supplementation of 500 uM potassium phosphate. Error bars represent one standard deviation from mean calculated from 3 biological replicates.

FIG. 4A, B, C, D show decreasing phosphate concentration led to expression of a GFP reporter protein of interest operably linked to control elements comprising the phosphate-sensitive Pliar53 promoter and promoters of the indicated pstS gene. Model soil bacterial strains are shown. The solid black line data series shows phosphate concentration over time while the dotted line data series shows per-cell GFP fluorescence over time. Error bars represent one standard deviation from mean calculated from 3 biological replicates.

FIG. 5 shows phosphate switch activation in the rhizosphere of corn inoculated with Azospirillum brasilense (pstS promoter controlling GFP expression) in a sterile system containing sand.

FIG. 6 shows phosphate concentration in sand in the rhizosphere of corn plants inoculated with Azospirillum brasilense (strain containing pstS promoter controlling GFP expression) in sterile conditions.

FIG. 7 shows a schematic of a gene expression cassette comprising a phosphate-sensitive pstS promoter operably linked to a gene encoding a GlnE protein lacking an adenylyl removing domain (uAT10). Constitutive expression of a gentamicin resistance gene (GmR) permits selection of bacterial strains comprising the cassette.

FIG. 8 shows de-repression of nitrogenase activity in engineered diazotrophs where glnE is replaced with a low Pi-inducible uAT.

FIG. 9 shows Pi-dependent ammonia release by Azospirillum brasilense Sp245 glnE mutants carrying chromosomally integrated pstS::uAT cassettes.

FIG. 10 shows metabolism and regulation of bacterial nitrogen fixation.

FIG. 11 shows low P concentration activated GFP expression in Kosakonia sacchari (Ks) and Klebsiella variicola (Kv) soil isolates engineered with a chromosomally integrated PpstS-GFP construct under simulated Midwestern United States field conditions (Illinois A, Illinois B, North Dakota, Oklahoma) in a plant growth room with controlled lighting, temperature, and humidity.

FIG. 12 shows the construct with a phosphate PpstS promoter driving expression of TetR repressor and Ptet driving expression of glnA where TetR represses expression of glnA (upper panel). The position of various RBS which were tested (BCD2, BCD13, and BCD22 for glnA; BCD2, BCD17, and BCD22 for TetR) in each transcription unit are shown as half circles (upper panel). FIG. 12 also shows ammonia production (mM NH₃/OD600) at 0 mM or 20 mM Pi for Kosakonia sacchari (Ks) strains with different constructs with various combinations of RBS in the glnA and tetR transcription units.

FIG. 13 shows the construct with a phosphate PpstS promoter driving expression of TetR repressor and Ptet driving expression of glnA where TetR represses expression of glnA (upper panel). The position of various RBS which were tested (BCD2, BCD13, and BCD22 for glnA; BCD2, BCD17, and BCD22 for TetR) in each transcription unit are shown as half circles (upper panel). FIG. 13 also shows ammonia production (mM NH₃/OD600) at 0 mM or 20 mM Pi for Rahnella aceris strains with different constructs with various combinations of RBS in the glnA and tetR transcription units.

FIG. 14 shows the construct with a phosphate PpstS promoter driving expression of TetR repressor and Ptet driving expression of glnA where TetR represses expression of glnA (upper panel). The position of various RBS which were tested in each transcription unit are shown as half circles (upper panel). FIG. 14 also shows ammonia production (mM NH3/OD600) at 0 mM or 20 mM Pi for Klebsiella variicola strains with different constructs with various combinations of RBS in the glnA and tetR transcription units.

FIG. 15 shows low-Pi inducible expression of GFP by PstS promoters in gamma-proteobacterial soil isolates (culture data). Rahnella aceris, Klebsiella variicola, and Kosakonia sacchari soil isolates were tested with either native PstS or heterologous PstS promoters fused to GFP.

FIG. 16 shows low-Pi inducible expression of GFP by two pstS promoters in Paenibacillus graminis. In the figure for each tested construct (Ppst1 GFP, Ppst2.1 GFP, Ppst2.2 GFP) or negative control (WT SM532), GFP expression normalized to the OD600 was determined from left to right as follows: 24 hours at 50 μM Pi, 24 hours at 0 μM Pi, 48 hours at 50 μM Pi, and 48 hours at 0 μM Pi.

FIG. 17 shows low-Pi control of a unidirectional adenylyl transferase (uAT) fused to the PstS promoter drives ammonia release (mM NH₃/OD600) for Kosakonia sacchari in liquid culture (left panel). The right panel shows results from a wild-type (WT) negative control lacking the PstS promoter fusion to the uAT.

FIG. 18A, B, C show the control GFP expression cassette of SEQ ID NO: 522 containing the BCD 2 vL ribosome binding site.

### DETAILED DESCRIPTION

Methods, genetically engineered bacteria (GEB), and related systems which use phosphate-sensitive promoters (PS-P) which are expressed under low phosphate conditions to drive expression of proteins or RNAs which are agriculturally relevant or which cause the production of at least one agriculturally relevant compound are disclosed herein. In plant growth media including soil, phosphate is consumed over time by plants and microorganisms. Once phosphate concentrations fall below a certain threshold level (e.g. about 50 μM, 40 μM, 30 μM, 20 μM, or 10 μM to about 1 μM), the desired production of proteins, RNAs, and/or agriculturally relevant compounds by the genetically engineered bacteria occurs. As the consumption of phosphate by the plants and microorganisms takes time, a desired delay in production of proteins, RNAs, and/or agriculturally relevant compounds by the genetically engineered bacteria is achieved. In certain embodiments, the genetically engineered bacteria are placed in the plant growth medium (e.g., soil) and then grow to a higher titer until the phosphate is consumed and expression of the protein or RNA of interest which is or causes the production of an agriculturally relevant compound is switched on. In comparison, other bacteria which have the protein or RNA of interest under the control of another promoter which is not phosphate-sensitive (e.g., a constitutive promoter) will express the protein, RNA, and/or compound before and/or shortly after placement in the plant growth media and will not grow to the higher titers achieved by the genetically engineered bacteria provided herein. Higher titers of the engineered bacteria provided herein can thus provide for higher titers of the desired proteins, RNAs, and/or agriculturally relevant compounds in the plant growth media in comparison to bacteria lacking the phosphate-sensitive promoter controlled genes.

Phosphate-sensitive promoter controlled gene expression systems provided herein are especially useful for the production of ammonia by bacteria which can act as bio-fertilizers. Constitutively active ammonia release can significantly inhibit bacteria growth as protein synthesis is effectively shut down. To obtain effective biofertilizers, bacteria provided herein are engineered to grow first to a high density in soil before the ammonia release mechanism is activated. The phosphate-sensing gene expression systems provided herein allow a desirable delay in ammonia production as phosphate is only slowly consumed by the plant and bacteria.

### Definitions

As used herein, the terms “about” or “approximately” indicate values slightly above or below the cited values, e.g., plus or minus 0.1% to 10% of the cited value.

As used herein, the phrase “agriculturally relevant compound” refers to a compound that provides for plant growth, altered plant morphology (e.g., increased branching/surface area of root systems), plant nutrition, plant growth regulation, or plant protection from abiotic (e.g., drought, salinity, cold, heat, or excess water stress) or biotic stress (e.g., plant pests including bacterial, fungal, insect, nematode, and viral plant pests).

As used herein, the phrase “agriculturally useful bacteria” or acronym “AUB” refers to bacteria which can grow in plant growth media (e.g., soil) and/or which can colonize crop plants

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrase “constitutive promoter” refers to a promoter, which is active under most growth and/or stationary phase conditions (e.g., in biofilms) in a given organism.

As used herein, the phrase “control element” refers to a promoter, a 5′ untranslated region (5′ UTR), a ribosome binding site, an enhancer, an insulator, a silencer, or a terminator. Control elements comprising a promoter, 5′ UTR, an enhancer, an insulator, and/or a silencer can contain transcriptional repressor binding sites, transcriptional activator binding sites, ribozymes, protein recognition sites, and/or sites for chemical modification of nucleobases.

The term “gene,” as used herein, refers to a hereditary unit consisting of a sequence of DNA located on a chromosome, plasmid, or other extra-chromosomal element that contains the genetic instruction for a particular characteristic or trait in an organism. The term “gene” thus includes a nucleic acid (for example, DNA or RNA) sequence that comprises coding and/or non-coding sequences necessary for the production of an RNA, a polypeptide, or a precursor of the RNA or protein. A functional polypeptide can be encoded by a full-length coding sequence or by any portion of the coding sequence as long as the desired activity or functional properties (e.g., enzymatic activity, DNA-binding activity, transcriptional activation, transcriptional repression, pesticidal activity, ligand binding, and/or signal transduction) of the polypeptide are retained.

The term “heterologous” is used herein to refer to any polynucleotide (e.g., DNA molecule) that has been introduced into a microorganism (e.g., a bacterium) where the polynucleotide is not sourced from the microorganism and/or has been inserted into a new location (e.g., in a distinct DNA sequence in the chromosome, plasmid, or other extrachromosomal element) in the microorganism (e.g., the bacterium). Non-limiting examples of heterologous DNA molecules that can be introduced into a microorganism include a non-naturally occurring (e.g., synthetic and/or recombinant) DNA molecule, a DNA molecule found in another microorganism (e.g., an intergeneric transfer of a DNA molecule comprising DNA from an organism of a different taxonomic genus), a DNA molecule found in another species (e.g., an intrageneric transfer of a DNA molecule comprising DNA from an organism of the same taxonomic genus), a DNA molecule found in a different location in the same species, and/or a DNA molecule found in the same strain or isolate of a species, where the DNA molecule has been inserted at a new location.

As used herein, the terms “include,” “includes,” and “including” are to be construed as at least having the features or items to which they refer while not excluding any additional unspecified features or items.

Unless otherwise stated, nucleic acid sequences in the text of this specification are given, when read from left to right, in the 5′ to 3′ direction. Nucleic acid sequences may be provided as DNA or as RNA, as specified; disclosure of a DNA or an RNA obtained therefrom also defines the exact complement of that DNA or RNA.

As used herein, the term “operably linked” refers to the association of nucleic acid sequences on a single nucleic acid fragment so that the function of one is or can be regulated by the other. For instance, a promoter is operably linked to an RNA or protein coding sequence if the promoter provides for transcription of the RNA or an mRNA encoding the protein.

As used herein, the term “phosphate,” when used in the context of phosphate concentration in a plant growth medium and/or phosphate concentrations which can activate a phosphate-sensitive promoter, refers to inorganic and soluble phosphate which is available to plants and microorganisms.

As used herein, the phrase “phosphate-sensitive promoter” refers to a promoter which is activated (e.g., up-regulated) or repressed (e.g., down-regulated) when phosphate concentration changes. In certain embodiments, a phosphate-sensitive promoter is activated or repressed when phosphate concentration crosses a threshold value. In certain embodiments, a phosphate-sensitive promoter is up-regulated or down-regulated proportionally in response to a change in phosphate concentration. In certain embodiments, a phosphate-sensitive promoter is activated when phosphate concentrations decrease below a threshold value of about 50 μM, 40 μM, 30 μM, 20 μM, or 10 μM to about 1 μM. In certain embodiments, a phosphate-sensitive promoter will be active at a concentration of phosphate from 0 μM to about 1 μM, 2 μM, 5 μM, 7 μM, or 10 μM.

As used herein, the term “refactored” refers to a gene, gene cluster, or operon that has been restructured. In some embodiments, restructuring may include changing a DNA coding sequence to a DNA sequence divergent from the wild-type gene while still encoding the same polypeptide. In some embodiments, restructuring may include computationally scanning genes to identify control elements, removing them, and optionally replacing them with different control elements. In some embodiments, “refactored” refers to a gene, gene cluster, or operon wherein the naturally-occurring promoter has been modified to contain a new promoter which exhibits different regulatory characteristics. Examples of such refactored gene clusters include refactored nf and/or fix gene clusters which allow nitrogenase to be expressed without transcriptional down-regulation by fixed nitrogen. Examples of refactoring methods and refactored nf gene clusters include those disclosed in U.S. patent Ser. No. 11/479,516, incorporated herein by reference in its entirety.

As used herein, the phrase “ribosome binding site” and corresponding acronym “RBS” refers to RNA comprising a Shine-Dalgarno sequence. In certain embodiments, a ribosome binding site can comprise a Shine-Dalgarno sequence and the translational initiation codon.

As used herein, the phrase “segment of a 5′ UTR” refers to one or more nucleotides of DNA encoding a 5′ UTR (5′ untranslated region) of a transcript and/or RNA comprising one or more nucleotides of a 5′ UTR. In certain embodiments, the segment of a 5′ UTR will comprise at least the first nucleotide of the 5′ UTR but can also comprise at least a Shine-Dalgarno sequence of a ribosome binding site in a 5′ UTR or the entire 5′ UTR.

As used herein, the phrase “transcriptional activator” refers to proteins or ribonucleoprotein (RNP) complexes capable of activating expression of a particular target gene. Transcriptional activators can thus include: (i) transcription factors comprising a DNA binding domain and a transcriptional activation domain; (ii) sigma factors which bind both a target promoter and RNA polymerase; and (iii) a catalytically inactive RNA-guided DNA binding protein which further comprises a transcriptional activator domain and a guide RNA which targets the RNP complex to the promoter.

Sequence identity or percent sequence identity can be measured with the BLASTN program (for nucleotide sequence percent identity determinations) or BLASTP program (for protein sequence percent identity determinations) using the BLAST (Basic Local Alignment Search Tool) available on the internet at blast.ncbi.nlm.nih.gov/Blast.cgi with default settings.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

In certain embodiments, one or more bacterial genes are down-regulated to cause the production of the agriculturally relevant compounds. Examples of bacterial genes which can be downregulated to produce ammonia include glnA, glnB, glnK, glnZ, nifL, and/or draT genes. Target glnA, glnB, glnK, glnZ, nifL, and/or draT gene sequences which can be down-regulated include those set forth in Table 5, in the sequence listing, and in US Patent Application No. US20210315212, which is incorporated herein by reference in its entirety. Target glnA, glnB, glnK, glnZ, nifL, or draT gene sequences which can be down-regulated further include sequences having at least 75%, 80%, 85%, 90%, 95%, or 99% sequence identity across the entire length of the sequences set forth in Table 1, in Table 5, in the sequence listing, and in US Patent Application No. US20210315212. Target glnA, glnB, glnK, glnZ, nifL, and/or draT gene sequences which can be down-regulated also include sequences which encode GnA, GnB, GnK, GnZ, NifL, or DraT proteins having at least 75%, 80%, 85%, 90%, 95%, or 99% sequence identity across the entire length of GlnA, GlnB, GlnK, GlnZ, NifL, or DraT proteins encoded by genes set forth in Table 1, in Table 5, in the sequence listing, and in US Patent Application No. Ue20210315212 or to GrnA, GlnB, GlnK, GlnZ, NifL, or DraT proteins in US Patent Application No. US20210315212.

TABLE 1
Non-limiting summary of target genes for down-regulation and ammonia production.

| Target | Representative | |
|---|---|---|
| Gene Name | Genes | Comments |
| amtB | | Down-regulation of the amtB gene can inhibit uptake of |
| | | ammonium from the extracellular environment and also |
| | | result in excretion of ammonia from the cells under |
| | | diazotrophic growth conditions. In certain embodiments of |
| | | the methods, systems, and genetically engineered bacteria |
| | | (GEB) provided herein, amtB down-regulation is combined |
| | | with down-regulation of glnA, glnB, glnK, glnZ, nifL, and/or |
| | | draT. |
| draT | | Nitrogenase is regulated by feedback repression at the |
| | | transcriptional and post translational level by ADP- |
| | | ribosylation via the DraT-DraG system. DraT catalyzes the |
| | | ADP-ribosylation of the nitrogenase Fe protein and shuts off |
| | | of nitrogenase under nitrogen excessive conditions, whereas |
| | | DraG catalyzes the removal of ADP ribose and reactivation |
| | | of nitrogenase under nitrogen starvation. Down-regulation of |
| | | DraT in a cell where nitrogenase is not regulated by feedback |
| | | repression at the transcriptional level can result in |
| | | nitrogenase activity that escapes feedback repression. In |
| | | certain embodiments of the methods, systems, and GEB |
| | | provided herein, draT down-regulation is combined with |
| | | down-regulation of glnA, amtB, glnB, glnK, glnZ, and/or |
| | | nifL. |
| glnA | Genes | Down-regulation of the glnA gene encoding glutamine |
| | encoding | synthetase can block assimilation of fixed nitrogen into |
| | the GlnA | glutamine, allow de-repression of nitrogen-fixing catalyst |
| | proteins | nitrogenase, and result in ammonia excretion. In certain |
| | of SEQ ID | embodiments of the methods, systems, and GEB provided |
| | NOs 229- | herein, glnA down-regulation is combined with down- |
| | 230, 460- | regulation of amtB, glnB, glnK, glnZ, nifL, and/or draT. |
| | 512, 519- | |
| | 521, 530- | |
| | 533 | |
| PII | | The PII proteins are the global nitrogen response regulators |
| (glnB, | | of the cell, acting on a suite of nitrogen metabolism proteins. |
| glnK, | | Down-regulation of PII genes or proteins can result in |
| glnZ) | | ammonia release. For example, down-regulation of PII genes |
| | | or proteins can force the adenylyl transferase (AT) to |
| | | adeny late the GS protein, leading to de-repressed nitrogenase |
| | | activity in the presence of ammonia. In certain embodiments |
| | | of the methods, systems, and GEB provided herein, PII |
| | | down-regulation (e.g., glnB, glnK, and/or glnZ down- |
| | | regulation), is combined with down-regulation of glnA, |
| | | amtB, nifL, and/or draT. |
| nifL | | The nifL gene is common amongst gamma-proteobacteria |
| | | and acts as an anti-activator of the nitrogenase master |
| | | regulator NifA. In conditions of low oxygen and low |
| | | glutamine, NifL represses activity of NifA, preventing |
| | | nitrogenase expression and nitrogen fixation. Down- |
| | | regulation of NifL can lead to increased NifA activity and |
| | | subsequently nitrogenase activity that is no longer repressed |
| | | by glutamine. Removal of nitrogenase feedback inhibition |
| | | leads to ammonia release. In certain embodiments of the |
| | | methods, systems, and GEB provided herein, nifL down- |
| | | regulation is combined with down-regulation of glnA, amtB, |
| | | glnB, glnK, glnZ, and/or draT and/or with upregulation of |
| | | nifA. |

In certain embodiments, one or more genes set forth in Table 1 can be down-regulated by one or more of the phosphate-sensitive systems or genes set forth in Table 2, Table 3, and/or Table 4. Examples of such combinations include down-regulation of GlnA (glutamine synthetase) by phosphate-sensitive promoter-controlled expression of a modified glnE gene encoding a GlnE protein lacking an adenylyl removing domain which exhibits unidirectional adenylyltransferase (uAT) activity. Particular embodiments of the systems for down-regulation of genes set forth in Table 1 are also disclosed within numbered embodiments 1-145.

Another example of such a combination is down-regulation of target genes set forth in Table 1 which include glnA (glutamine synthetase; e.g., as in Table 1 and SEQ ID NO: 229-230, 460-512, 519-521, 530-533) through phosphate-sensitive promoter (P-SP) controlled expression of a repressor protein (e.g., TetR repressor) which binds a natural or synthetic DNA motif in the promoter and/or 5′ UTR of the target gene (e.g., glnA). In certain embodiments, the P-SP is operably linked to DNA encoding a transcript comprising a relatively weak ribosome binding site (RBS) which is operably linked to the transcriptional repressor protein coding region and supports lower levels of repressor protein expression in comparison to strong RBS. Relatively weak RBS include the BCD22 (SEQ ID NO: 449) RBS set forth herein and in Mutalik et al., 2013, doi:10.1038/nmeth.2404) as well as the BCD8 and BCD24 RBS set forth in Mutalik et al., 2013, doi:10.1038/nmeth.2404. Relatively weak RBS can also comprise other procaryotic RBS binding sites (e.g., obtainable from alpha-, beta-, or gamma-proteobacteria) comprising modifications of the Shine-Dalgarno 2 (SD2) sequence of the RBS that reduce translational initiation of operably linked protein coding regions (Mutalik et al., 2013, doi:10.1038/nmeth.2404). Additional weak RBS binding sites are disclosed in Wen et al., 2020, doi.org/10.1080/15476286.2020.1861406. Relatively strong RBS include the BCD2vL RBS encoded by SEQ ID NO: 430 and disclosed in Mutalik et al., 2013. A green fluorescent protein (GFP) reporter gene cassette where the strong BCD2vL RBS encoded by SEQ ID NO: 430 is operably linked to a promoter and the GFP reporter is set forth in SEQ ID NO: 522. Relatively weak RBS suitable for operable linkage to the transcriptional repressor protein coding region thus include RBS which when substituted in place of the BCD2vL RBS located at nucleotides 723 to 810 of control green fluorescent protein (GFP) reporter gene expression cassette of SEQ ID NO: 522 provide 0.1% to 44% of GFP reporter gene expression cassette of SEQ ID NO: 522 when expressed under otherwise identical conditions. In certain embodiments, RBS suitable for operable linkage to the transcriptional repressor protein coding region include RBS which when substituted in place of the BCD2vL RBS located at nucleotides 723 to 810 of control green fluorescent protein (GFP) reporter gene expression cassette of SEQ ID NO: 522 provide (i) 0.1%, 0.5%, 1%, 2%, 3%, 4%, 5%, 6%, 7%, or 8% to 10%, 15%, 20%, 25%, 30%, 35%, 40%, or 44%; or (ii) 10%, 15%, or 20% to 25%, 30%, 35%, 40%, or 44%; of GFP reporter gene expression cassette of SEQ ID NO: 522 when expressed under otherwise identical conditions. In certain embodiments, determination of percent (%) operably linked reporter gene expression for any given test candidate RBS relative to the SEQ ID NO: 430 RBS can be determined by: (i) inserting the test candidate RBS and the control strong RBS (SEQ ID NO: 430) at the same position (e.g., nucleotides 723 to 810) in the 5′ UTR of the same reporter gene in the test candidate gene expression cassette and control gene expression cassette (e.g., SEQ ID NO: 522), where the only difference between the test candidate and control gene expression cassette is the RBS sequence (i.e., the promoters, 5′ UTR, reporter gene, and terminator are otherwise identical); (ii) recovering transformed bacteria where the test candidate and control gene expression cassette are both inserted at the same position and orientation in the bacterial chromosome of an otherwise identical bacterial strain; and (iii) growing the transformed bacteria with the test candidate and control gene expression cassette under identical conditions, harvesting the bacteria at the same density during exponential growth (e.g., in mid-exponential growth phase), and assaying for reporter gene expression in identical assays. In certain embodiments, the bacteria comprising the test candidate gene expression cassette with a suitable weak RBS will provide 0.1%, 0.5%, 1%, 2%, 3%, 4%, 5%, 6%, 7%, or 8% to 10%, 15%, 20%, 25%, 30%, 35%, 40%, or 44% of the reporter gene expression provided by the control gene cassette (e.g., the GFP reporter gene expression cassette of SEQ ID NO: 522) having the strong RBS (SEQ ID NO: 430).

Target genes (e.g., a target gene of Table 1 such as glnA) that are down-regulated by phosphate-sensitive promoter (P-SP) controlled expression of a repressor protein (e.g., TetR or Lac repressor) can be modified in host bacteria to provide a host where the functional copy of the target gene is under control of the repressible promoter. In certain embodiments, the endogenous target gene is disrupted (e.g., contains an insertion, deletion, and/or substitution which results in a null allele of the endogenous target gene) and a modified target gene comprising a repressible promoter (e.g., TetR repressible promoter) which is operably linked to a target gene coding region (e.g., a glnA gene from the host bacterium or another source) is inserted in the chromosome of the host bacterium. In other embodiments, the target gene (e.g., a target gene of Table 1 such as glnA) is encoded by the endogenous target gene and the endogenous target gene (e.g., glnA) is operably linked to the promoter which is repressed by the repressor protein (e.g., a TetR or LacI repressor). Examples of such embodiments include those where endogenous glnA gene promoter which is operably linked to the endogenous glnA gene coding region is replaced with a promoter that is under control of the repressor. In certain embodiments, the target gene (e.g., the entire promoter and coding region of a glnA gene in a target bacterium such as a Kosakonia sp.) is substituted with a heterologous target gene comprising the repressible promoter (e.g., TetR repressible promoter) which is operably linked to a heterologous target gene coding region (e.g., a glnA gene from a bacterium other than Kosakonia sp. or variant thereof including an Azospirillum glnA coding region). Suitable promoters under control of the repressor include: (i) recombinant target gene (e.g., glnA) promoter containing a natural or synthetic DNA motif inserted within the promoter at a position which will result in inhibition of transcription when bound by the repressor; or (ii) wild-type promoters or variants thereof which are repressed by the repressor protein (e.g., a Tet operon promoter which is repressed by a TetR repressor protein).

TABLE 2
Non-limiting summary of systems for down-regulating
target genes under decreased phosphate concentrations

| | | Non-limiting |
|---|---|---|
| | | descriptions of |
| | | materials and |
| | Non-limiting materials which | methods which can |
| | can be used to implement | be adapted for use |
| System | system | in the mechanism |
| Phosphate-sensitive | P-SP described in Table 5, SEQ ID | Ramos et al., 2005, |
| promoter (P-SP) | NO: 417-428, 515, 517, or 518; | doi.org/10.1128/mmbr.69.2.326- |
| controlled expression of a | Lambda phage repressor (cI) | 356.2005 |
| repressor protein which | (SEQ ID NO: 26), the tet | |
| binds a natural or synthetic | repressor (TetR) (SEQ ID NO: | |
| DNA motif in the | 27, the lac repressor (LacI) SEQ | |
| promoter and/or 5′ UTR of | ID NO: 29, PhIF (SEQ ID NO: | |
| a target gene. | 30), (e.g., in Table 5), a | |
| | catalytically inactive RNA- | |
| | guided DNA binding protein, | |
| | proteins comprising a DNA- | |
| | binding zinc finger domain, a | |
| | transcription activator-like | |
| | effector (TALE) lacking a | |
| | transcription activation domain | |
| | (TAD), and/or any variant | |
| | thereof. | |
| P-SP controlled expression | Lambda phage repressor (cl), | Hooshangi et al., |
| of a repressor which | the tet repressor (TetR), the lac | 2004, doi: |
| controls a second gene | repressor (LacI), and PhIF (e.g., | 10.1073pnas.0408507102; |
| encoding a second | in Table 5) | Ramos et |
| repressor in a repressor | | al., 2005, |
| cascade comprising an odd | | doi.org/10.1128/mmbr.69.2.326- |
| number (1, 3, 5, 7, 9, or | | 356.2005 |
| more) of repressors and | | |
| repressed genes encoding | | |
| repressors, where the last | | |
| repressor in the cascade | | |
| can repress expression of | | |
| the gene targeted for down | | |
| regulation when the P-SP | | |
| is active. | | |
| P-SP controlled expression | P-SP described in Table 5, SEQ | Arroyo-Olarte et al. |
| of a non-coding synthetic | ID NO: 417-428; Guide RNA | 2021, doi: |
| small RNA (sRNA) | molecules that hybridize to a | 10.3390/microorganisms9040844. |
| | DNA sequence in target gene | Cytosine base |
| | and allow: (1) site-specific | editing: |
| | cleavage by an RNA-guided | U.S. Pat. No. 9,840,699B2 |
| | DNA endonuclease (e.g., Cas9 | incorporated herein |
| | or Cas12); (2) site-specific | by reference in its |
| | binding and transcriptional | entirety. |
| | repression by a protein | Adenosine base |
| | comprising dCas9 or dCas12, | editing: |
| | (3) site-specific base editing | U.S. Pat. No. 10,113,163B2 |
| | with a catalytically inactive | incorporated herein |
| | RNA-guided DNA binding | by reference in its |
| | protein coupled to a cytidine or | entirety. |
| | adenine deaminase, (4) site- | Prime editing: |
| | specific chemical modification | U.S. Pat. No. 11,643,652B2 |
| | of DNA nucleobases (including | incorporated herein |
| | methylation, demethylation, | by reference in its |
| | hydroxymethylation, | Entirety. |
| | carboxymethylation) by a | Prime editing: |
| | catalytically inactive RNA- | U.S. Pat. No. 11,643,652B2 |
| | guided DNA binding protein | incorporated herein |
| | coupled to a DNA modifying | by reference in its |
| | enzyme (including | entirety. |
| | methyltansferases, TET | Site-specific DNA |
| | enzymes, and | methylation: doi: |
| | carboxymethylation enzymes | 10.1016/j.cell.2016.08.056 |
| | such as M.MpeI N374K), or (5) | DNA |
| | site-specific DNA mutagenesis | carboxymethylation: |
| | with a catalytically inactive | doi: |
| | RNA-guided DNA binding | 10.1016/j.chembiol.2020.09.006. |
| | protein coupled to a reverse | |
| | transcriptase or error-prone | |
| | DNA polymerase. | |
| P-SP controlled expression | P-SP described in Table 5, SEQ | |
| of a non-coding synthetic | ID NO: 417-428, 515, 517, or | |
| small RNA (sRNA) | 518; RNA molecules that | |
| | hybridize to an RNA sequence | |
| | expressed by target gene | |
| P-SP controlled expression | P-SP described in Table 5, SEQ | Good L, Stach J E. |
| of a non-coding synthetic | ID NO: 417-428, 515, 517, or | Front Microbiol. |
| small RNA (sRNA) | 518; Expression of sRNA which | 2011, doi: |
| | hybridize to target gene mRNA | 10.3389/fmicb.2011.00185 |
| | Shine-Dalgarno sequence of a | Lee J L, Moon T S. |
| | ribosome binding site and/or | Methods. 2018, doi: |
| | start codon | 10.1016/j.ymeth.2018.01.001 |
| | | Na D et al. Nat |
| | | Biotechnol. 2013, |
| | | doi: |
| | | 10.1038/nbt.2461 |
| P-SP controlled expression of a | P-SP described in Table 5, SEQ ID | U.S. Pat. No. |
| site-specific recombinase (SSR) | NO: 417-428, 515, 517, or 518; | 10,614,353, |
| or integrase protein (INTP) | SSR, INTP, and SSRRS including | incorporated herein |
| which can excise a target gene or | PhiC31 serine integrase, IntS, IntM, | by reference in its |
| region within the target gene | IntG - ICEMcSym 1271, YdcL - | entirety. |
| flanked by site-specific | ICEBs, or Int - ICE SXT/R39 | |
| recombinase recognition sites | integrase, and corresponding attL | |
| (SSRRS) in a direct | and attR sites and the genetically | |
| configuration | engineered bacterium comprises a | |
| | gene encoding a recombinase | |
| | directionality factor (RDF); FLP | |
| | recombinase and FRT sites or Cre- | |
| | recombinase and loxP sites. | |
| P-SP controlled expression | P-SP described in Table 5, SEQ | U.S. Pat. No. |
| of a site-specific | ID NO: 417-428, 515, 517, or | 10,614,353, |
| recombinase (SSR) or | 518; SSR, INTP, and SSRRS | incorporated herein |
| integrase protein (INTP), | including: PhiC31 serine | by reference in its |
| an integrative SSRRS in | integrase, IntS, IntM, IntG - | entirety. |
| the target gene, and an | ICEMcSym 1271, YdcL - | |
| integrative element | ICEBs, or Int - ICE SXT/R39 | |
| comprising an SSRRS that | integrase, and corresponding | |
| provides for insertional | recognition sites (attB and attP) | |
| inactivation. | including those in Table 5; FLP | |
| | recombinase and FRT sites; or | |
| | Cre-recombinase and loxP sites. | |
| P-SP controlled expression | P-SP described in Table 5, SEQ | U.S. Pat. No. |
| of a site-specific | ID NO: 417-428, 515, 517, or | 10,614,353, |
| recombinase (SSR) or | 518; SSR, INTP, and SSRRS | incorporated herein |
| integrase protein (INTP), | including: PhiC31 serine | by reference in its |
| in a cell comprising an | integrase, IntS, IntM, IntG - | entirety. |
| SSR- or integrase- | ICEMcSym 1271, YdcL - | Siuti et al. 2013, |
| controlled promoter switch | ICEBs, or Int - ICE SXT/R39 | doi: |
| where inversion of | integrase, and corresponding | 10.1038/nbt.2510 |
| promoter in switch by SSR | recognition sites, including | Bernabé-Orts et al. |
| or INTP inverts promoter | those in Table 5; FLP | 2020, |
| and down-regulates gene. | recombinase and FRT sites, or | doi.org/10.1093/nar/gkaa104 |
| | Cre-recombinase and loxP sites. | |
| | Constitutive and inducible | |
| | promoters used in the SSR or | |
| | integrase controlled promoter | |
| | switch. | |
| P-SP controlled | P-SP described in Table 5, SEQ ID | Arroyo-Olarte et al. |
| expression of a site- | NO: 417-428, 515, 517, or 518; RNA- | 2021, doi: |
| specific RNA-guided | guided RNA endonuclease (e.g., Cas | 10.3390/microorganisms9040844; |
| RNA endonuclease which | Type III systems and Cas Type VI | Gasiunas et al. |
| specifically cleaves one or | systems including Cas13) | 2014, doi: |
| more RNA transcripts of | | 10.1007/s00018- |
| the target gene. | | 013-1438-6; |
| | | Hale et al. 2012, |
| | | doi: 10.1016/j.molcel.2011.10.023; |
| | | Hillary and Ceasar, |
| | | 2022, doi: |
| | | 10.1007/s12033- |
| | | 022-00567-0; |
| | | U.S. Pat. No. |
| | | 11,788,083, |
| | | incorporated herein |
| | | by reference in its |
| | | entirety. |
| P-SP controlled | P-SP described in Table 5, SEQ ID | Arroyo-Olarte et al. |
| expression of a site- | NO: 417-428, 515, 517, or 518; RNA- | 2021, doi: |
| specific DNA | guided DNA endonuclease, a protein | 10.3390/microorganisms9040844. |
| endonuclease which | comprising a DNA-binding zinc | Hillary and Ceasar, |
| specifically cleaves one or | finger domain and endonuclease | 2022, doi: |
| more DNA sequences in | domain, a transcription activator-like | 10.1007/s12033- |
| the target gene. | effector (TALE) and endonuclease | 022-00567-0. |
| | domain, a meganuclease, a homing | |
| | endonuclease, or a restriction | |
| | endonuclease | |
| P-SP controlled expression | P-SP described in Table 5, SEQ | |
| of a protease which | ID NO: 417-428, 515, 517, or | |
| cleaves a protease specific | 518; TEV, TVMV, SMMV, | |
| recognition sequence | TrMV, SMV, PPV, HCV, | |
| (PSRS) in the target gene. | enterokinase, Factor Xa, furin | |
| | proteases (e.g., SEQ ID Nos: 45, | |
| | 46, 47, 48, 49, 50, 51, 52, and | |
| | 53) and corresponding PSRS. | |
| P-SP controlled expression | P-SP described in Table 5, SEQ | |
| of a ClpAP ATP- | ID NO: 417-428, 515, 517, or | |
| dependent protease and a | 518; ClpAP ATP-dependent | |
| ClpS Leu/N-recognin | protease and a ClpS Leu/N- | |
| where the target gene | recognin | |
| protein has an N- | | |
| terminal -Leu, -Phe, -Trp, | | |
| or -Tyr residue | | |
| P-SP controlled expression | P-SP described in Table 5, SEQ | |
| of a protease which | ID NO: 417-428, 515, 517, or | |
| cleaves a PSRS in the N- | 518; TEV, TVMV, SMMV, | |
| terminus of the target gene | TrMV, SMV, PPV, HCV, | |
| to expose an N- | enterokinase, Factor Xa, furin | |
| terminal -Leu, -Phe, -Trp, | proteases and corresponding | |
| or -Tyr residue, allowing | PSRS. | |
| degradation by native ClpS | | |
| and ClpAP. | | |
| P-SP controlled expression | P-SP described in Table 5, SEQ | Pinto et al., doi: |
| of a transcriptional | ID NO: 417-428, 515, 517, or | 10.1093/nar/gky614 |
| activator (TA) which | 518; tet responsive element- | |
| upregulates expression of a | binding TA transcription | |
| protease which cleaves a | factor, or a transcriptional | |
| PSRS in the target gene. | activator domain fused to a | |
| | DNA targeting protein or | |
| | ribonucleoprotein (e.g., DNA | |
| | sequence-specific binding | |
| | protein or ribonucleoprotein). | |
| | TA also include sigma factors | |
| | which bind the core promoter | |
| | and RNA polymerase. | |
| P-SP controlled expression | P-SP described in Table 5, SEQ | |
| of a repressor which | ID NO: 417-428, 515, 517, or | |
| inhibits expression of a | 518; N-terminal tag which | |
| protease which activates | renders the target protein | |
| the target gene by cleaving | inactive by (i) sterically | |
| a PSRS located C-terminal | blocking the active site, (ii) | |
| to an N-terminal amino | binding the active site, or (iii) | |
| acid tag designed to render | preventing multimer formation. | |
| the target protein inactive. | | |
| P-SP controlled expression | P-SP described in Table 5, SEQ | Muyldermans 2013, |
| of a nanobody which binds | ID NO: 417-428, 515, 517, or | doi: |
| and inactivates the target | 518; Nanobodies directed | 10.1146/annurev- |
| protein. | against GlnA, GlnB, GlnK, | biochem-063011- |
| | GlnZ, NifL, or DraT proteins | 092449 |
| P-SP controlled expression | P-SP described in Table 5, SEQ | |
| of an aptamer which binds | ID NO: 417-428, 515, 517, or | |
| and inactivates the target | 518; Aptamers directed against | |
| protein. | glnA, glnB, glnK, glnZ, nifL, or | |
| | draT genes, gene transcripts, or | |
| | encoded proteins. | |

In certain embodiments, one or more bacterial genes are up-regulated to cause the production of the agriculturally relevant compounds. Examples of bacterial genes which can be up-regulated to produce ammonia include nifA, ntrC, glnR, a gene encoding a glutaminase enzyme, a gene encoding a GlnE protein lacking an adenylyl removing domain which exhibits unidirectional adenylyltransferase (uAT) activity (also referred to herein as GlnE-uAT), one or more nif cluster gene(s), and/or one or more fix cluster gene(s). Target nifA, ntrC, glnR, glutaminase encoding genes, GlnE-uAT genes, nif cluster gene(s), and/or fix cluster gene sequences which can be up-regulated include those set forth in Table 3, in the sequence listing, and in US Patent Application No. US20210315212, which is incorporated herein by reference in its entirety. Target nifA, ntrC, glnR, glutaminase encoding genes, GlnE-uAT genes, nif cluster gene(s), and/or fix cluster genes which can be up-regulated further include sequences having at least 75%, 80%, 85%, 90%, 95%, or 99% sequence identity across the entire length of the sequences set forth in Table 3, in the sequence listing, and in US Patent Application No. US20210315212. Target nifA, ntrC, glnR, glutaminase encoding genes, GlnE-uAT genes, nif cluster gene(s), and/or fix cluster gene sequences which can be up-regulated also include sequences which encode NifA, NtrC, GlnR, glutaminase, GlnE-uAT, nif cluster, and/or fix cluster proteins having at least 75%, 80%, 85%, 90%, 95%, or 99% sequence identity across the entire length of NifA, NtrC, GlnR, glutaminase, GlnE-uAT, nif cluster, and/or fix cluster proteins encoded by genes set forth in Table 3, in the sequence listing, and/or in US Patent Application No. US20210315212 or to NifA, NtrC, GlnR, Glutaminase, GlnE-uAT, nif cluster, and/or fix cluster proteins in Table 3, in Table 5, in the sequence listing, and/or in US Patent Application No. US20210315212.

In certain embodiments, one or more bacterial genes are up-regulated to cause the production of phosphate from insoluble forms of phosphate (e.g., present in plant growth media). Examples of bacterial genes which can be up-regulated to produce phosphate include genes encoding a phytase enzyme (e.g., a cysteine phytase, a histidine acid phytase, or a beta-propeller phytase), genes encoding an acid phosphatase enzyme (e.g., an acpA, aphA, phoC, napA, napD, or napE gene), and/or genes encoding a protein which stimulates organic acid release from the bacterium, (e.g., a gluconate dehydrogenase (GAD), a glucose dehydrogenase (GDH), or a pyrroloquinoline (PQQ) synthase encoding gene). Target phytase-, acid phosphatase- (e.g., an acpA, aphA, phoC, napA, napD, or napE gene), GAD-, GDH-, or pyrroloquinoline (PQQ) synthase-encoding genes which can be up-regulated include those set forth in Table 3, in the sequence listing, and in US Patent Application No. US20210345618, which is incorporated herein by reference in its entirety. Target phytase-, acid phosphatase- (e.g., an acpA, aphA, phoC, napA, napD, or napE gene), GAD-, GDH-, or pyrroloquinoline (PQQ) synthase-encoding genes which can be up-regulated further include sequences having at least 75%, 80%, 85%, 90%, 95%, or 99% sequence identity across the entire length of the sequences set forth in Table 3, in the sequence listing, and in US Patent Application No. US20210345618. Target phytase-, acid phosphatase- (e.g., an acpA, aphA, phoC, napA, napD, or napE gene), GAD-, GDH-, or pyrroloquinoline (PQQ) synthase-encoding genes which can be up-regulated also include sequences which encode phytase, acid phosphatase (e.g., an AcpA, AphA, PhoC, NapA, NapD, or NapE protein), GAD, GDH, or pyrroloquinoline (PQQ) synthase proteins having at least 75%, 80%, 85%, 90%, 95%, or 99% sequence identity across the entire length of phytase, acid phosphatase (e.g., an AcpA, AphA, PhoC, NapA, NapD, or NapE protein), GAD, GDH, or pyrroloquinoline (PQQ) synthase proteins encoded by genes set forth in Table 3, in the sequence listing, and in US Patent Application No. US20210345618 or to phytase, acid phosphatase (e.g., an AcpA, AphA, PhoC, NapA, NapD, or NapE protein), GAD, GDH, or pyrroloquinoline (PQQ) synthase proteins in Table 3, in the sequence listing, and/or in US Patent Application No. US20210345618.

TABLE 3
Non-limiting summary of target genes for up-regulation and
agriculturally relevant compounds produced by up-regulation

| Target Gene or | | |
|---|---|---|
| Target Gene | Agri- | |
| encoded protein | culturally | |
| (non-limiting | relevant | |
| examples) | compound | Comment |
| nifA | ammonia | NifA is the master transcriptional regulator of |
| | | genes involved in nitrogenase assembly, function |
| | | and maintenance (nif, fix). The nifA gene in |
| | | bacteria is typically regulated at the transcriptional |
| | | and often posttranslational level by nitrogen, |
| | | oxygen, and in some cases, carbon. In Azotobacter |
| | | vinlandii, overexpression of nifA from a |
| | | heterologous promoter drives constitutive |
| | | nitrogenase activity and stimulates ammonia |
| | | release. In certain embodiments, nifA up- |
| | | regulation is combined with: (i) nifL down- |
| | | regulation (e.g., mutants that are unable to regulate |
| | | NifA at the protein level); (ii) in draT down- |
| | | regulation; and/or (iii) nif/fix gene cluster up- |
| | | regulation. In certain embodiments of the methods, |
| | | systems, and GEB provided herein, nifA up- |
| | | regulation is combined with: (i) down-regulation |
| | | of glnA, amtB, glnB, glnK, glnZ, nifL, and/or draT; |
| | | and/or (ii) up-regulation of ntrC, glnR, uAT, GLS, |
| | | one or more nif cluster gene(s), and/or one or more |
| | | fix cluster genes |
| ntrC | ammonia | Kukolj et al., 2020, |
| | | doi:: 10.1021/acs.jproteome.9b00397 |
| glnR | ammonia | The glnR gene acts as a regulator of nitrogen |
| | | metabolism genes in gram-positive bacteria. Up- |
| | | regulation of glnR can drive nitrogenase activity |
| | | and promote ammonia release. In certain |
| | | embodiments of the methods, systems, and GEB |
| | | provided herein, glnR up-regulation is combined |
| | | with: (i) down-regulation of glnA, amtB, glnB, |
| | | glnK, glnZ, nifL, and/or draT; and/or (ii) up- |
| | | regulation of nifA, ntrC, uAT, GLS, one or more |
| | | nif cluster gene(s), and/or one or more fix cluster |
| | | genes to promote ammonia release. |
| nif/fix cluster | ammonia | The nif/fix genes are required for iron molybdenum |
| genes | | nitrogenase assembly, function, and maintenance. |
| | | Up-regulation of nif/fix cluster genes can promote |
| | | ammonia release. In certain embodiments of the |
| | | methods, systems, and GEB provided herein, up- |
| | | regulation of one or more nif cluster gene(s) and/or |
| | | one or more fix cluster genes is combined with: (i) |
| | | down-regulation of glnA, amtB, glnB, glnK, glnZ, |
| | | nifL, and/or draT; and/or (ii) up-regulation of nifA, |
| | | ntrC, glnR, GLS, and/or a uAT. |
| GlnE protein lacking | ammonia | Up-regulation of GlnE-uAT proteins can result in |
| an adenylyl removing | | the down-regulation of glutamine synthetase by |
| domain which | | adenylation and result in ammonia release. In |
| exhibits | | certain embodiments of the methods, systems, and |
| unidirectional | | GEB provided herein, uAT up-regulation is |
| adenylyltransferase | | combined with: (i) down-regulation of glnA, |
| (uAT) activity (GlnE- | | amtB, glnB, glnK, glnZ, nifL, and/or draT; and/or |
| uAT protein). GlnE- | | (ii) up-regulation of nifA, ntrC, glnR, GLS, one or |
| uAT proteins include | | more nif cluster gene(s), and/or one or more fix |
| SEQ ID NO: 16-25; | | cluster genes to promote ammonia release. |
| GlnE-uAT encoding | | |
| genes include SEQ | | |
| ID NO: 1-15. | | |
| Glutaminase (GLS) | ammonia | GLS enzymes catalyze the conversion of |
| enzymes | | glutamine into glutamate. Up-regulation of GLS |
| | | can reduce glutamine levels in the cell and force a |
| | | nitrogen starvation response, resulting in de- |
| | | repression of nitrogenase and promoting nitrogen |
| | | release. In certain embodiments of the methods, |
| | | systems, and GEB provided herein, GLS up- |
| | | regulation is combined with: (i) down-regulation |
| | | of glnA, amtB, glnB, glnK, glnZ, nifL, and/or draT; |
| | | and/or (ii) up-regulation of nifA, ntrC, glnR, uAT, |
| | | one or more nif cluster gene(s), and/or one or more |
| | | fix cluster genes. |
| Acid phosphatases | phosphate | Acid phosphatases catalyze dephosphorylation of |
| (e.g., acpA, phoC, | | phosphor-ester or phospho-anhydride bond of organic |
| napA, napD, napE) | | compounds to release phosphate. Acid phosphatase gene |
| | | sources include genes in US20210345618A1, |
| | | incorporated herein by reference in its entirety. |
| aphA | phosphate | |
| phytase (including a | phosphate | Phytase-mediated hydrolysis of phytic acid in |
| cysteine phytase, | | different positions of the inositol ring to release |
| histidine acid | | phosphorus, zinc, |
| phytase, or beta- | | and other minerals in inorganic form; |
| propeller phytase; | | US20210345618A1 |
| including phytases in | | |
| Table 5 and phytases | | |
| comprising a protein | | |
| sequence having at | | |
| least 70%, 75%, 80%, | | |
| 85%, 90%, 95%, | | |
| 98%, or 99% | | |
| sequence identity to | | |
| SEQ ID NO: 58-138, | | |
| or 139. | | |
| gluconate | phosphate | Conversion of the insoluble inorganic phosphate to |
| dehydrogenase | | plant-available phosphate through H+ excretion |
| (GAD) | | and organic acid production. GAD gene sources |
| | | include genes in US20210345618A1, incorporated |
| | | herein by reference in its entirety. |
| glucose | phosphate | Conversion of the insoluble inorganic phosphate to |
| dehydrogenase | | plant-available phosphate through H+ excretion |
| (GDH) | | and gluconic acid production. |
| gluconic acid | phosphate | Conversion of the insoluble inorganic phosphate to |
| biosynthetic gene(s) | | plant-available phosphate through H+ excretion |
| (e.g., | | and gluconic acid production. Gluconic acid |
| pyrroloquinoline | | biosynthetic genes include pqqA, pqqB, pqqC, |
| (PQQ) synthase | | pqqD, pqqE, gcd, gabY, variants thereof, and |
| | | combinations thereof disclosed in |
| | | US20210345618A1, incorporated herein by |
| | | reference in its entirety. |
| B. thuringiensis Cry | Protein | Cry1, Cry2, Cry3, Cry4, Cry5, Cry6, Cry7, Cry8, |
| endotoxins, variants | based | Cry9, Cry10, Cry11, Cry12, Cry13, Cry14, Cry15, |
| thereof, and chimeras | insecticide | Cry16, Cry17, Cry18, Cry19, Cry20, Cry21, |
| thereof | | Cry22, Cry23, Cry24, Cry25, Cry26, |
| | | Cry27, Cry28, Cry29, Cry30, Cry31, Cry32, |
| | | Cry33, Cry34, Cry35, Cry36, Cry37, Cry38, |
| | | Cry39, Cry40, Cry41, Cry42, Cry43, Cry44, |
| | | Cry45, Cry46, Cry47, Cry49, Cry51, Cry52, |
| | | Cry53, Cry 54, Cry55, Cry56, Cry57, Cry58, Cry59. |
| | | Cry60, Cry61, Cry62, Cry63, Cry64, Cry65, |
| | | Cry66, Cry67, Cry68, Cry69, Cry70 and |
| | | Cry71 endotoxins, variants thereof, and chimeras |
| | | thereof, including those disclosed in US Patent |
| | | Application No. US20210315212, incorporated |
| | | herein by reference in its entirety |
| B. thuringiensis Cyt | Protein | B. thuringiensis cytolytic cyt1 and cyt2 genes |
| proteins | based | including those disclosed in US Patent Application |
| | insecticide | No. US20210315212, incorporated herein by |
| | | reference in its entirety. |
| VIP (vegetative | Protein | VIP1, VIP2, VIP3 proteins disclosed in U.S. Pat. |
| insecticidal proteins) | based | Nos. 5,877,012, 6,107,279 6,137,033, 7,244,820, |
| toxins | insecticide | 7,615,686, and 8,237,020, incorporated herein by |
| | | reference in their entireties. |
| Toxin complex (TC) | Protein | TC Class A proteins including TcbA, TcdA, |
| proteins, obtainable | based | XptA1 and XptA2; TC Class B proteins including |
| from organisms such | insecticide | TcaC, TcdB, XptB1Xb and XptC1; TC Class C |
| as Xenorhabdus, | | proteins including TccC, XptClXb and XptBl Wi |
| Photorhabdus and | | (U.S. Pat. Nos. 7,491,698 and 8,084,418, |
| Paenibacillus | | incorporated herein by reference in their |
| | | entireties). |
| Anti-microbial | Protein- | Defensin and defensin-multimers disclosed in US |
| proteins including | based | Patent Applic. Publ. No. 20190185877, |
| defensin proteins | fungicides | incorporated herein by reference in its entirety. |
| Genes encoding | RNA based | dsRNA directed against insect target proteins |
| dsRNA to produce | insecticides | including those disclosed in U.S. Pat. No. |
| RNAi response | | 11,312,975, incorporated herein by reference in its |
| directed against | | entirety. |
| insect target genes | | |
| Genes encoding | RNA based | dsRNA directed against fungal target proteins |
| dsRNA to produce | fungicides | including those disclosed in U.S. Pat. No. |
| RNAi response | | 8,865,968, incorporated herein by reference in its |
| directed against | | entirety. |
| fungal target genes | | |
| Genes encoding | RNA based | dsRNA directed against nematode target proteins |
| dsRNA to produce | nematicides | including those disclosed in U.S. Pat. No. |
| RNAi response | | 9,388,409, incorporated herein by reference in its |
| directed against | | entirety. |
| nematode target | | |
| genes | | |
| gluconate | Zinc | Conversion of the insoluble zinc to plant-available |
| dehydrogenase | | zinc through H+ excretion and organic acid |
| (GAD), optionally in | | production (Eshagi et al., Iran J Microbiol. 2019 |
| strain selected for | | October; 11(5): 419-430). GAD gene sources include |
| zinc solubilization | | genes in US20210345618A1, incorporated herein |
| activity | | by reference in its entirety. |
| gluconate | Potassium | Conversion of the insoluble potassium to plant- |
| dehydrogenase | | available zinc through H+ excretion and organic |
| (GAD), optionally in | | acid production (Kumar etal., 2013, DOI: |
| a strain selected for | | 10.1007/s00284-013-0372-z; Rajawat et al., 2016, |
| potassium | | doi: 10.1016/S1002-0160(15)60080-7). GAD |
| solubilization activity | | gene sources include genes in US20210345618A1, |
| | | incorporated herein by reference in its entirety. |
| Genes encoding | Iron | Production of catecholates and phenolates, |
| siderophore | | hydroxamates, carboxylates, and mixed-type |
| biosynthetic and | | siderophores which chelate iron and permit uptake |
| transport proteins | | by plants; Timofeeva et al., 2022, doi: |
| including | | 10.3390/plants11223065 |
| dnbACDEBF gene | | |
| clusters, genes | | |
| encoding non- | | |
| ribosomal peptide | | |
| synthetase (NRPS), | | |
| polyketide synthase | | |
| (PKS), and NRPS- | | |
| independent | | |
| siderophore | | |
| synthetase (NIS); and | | |
| major facilitator | | |
| superfamily | | |
| (MFS) transporters | | |
| (ymfE), TonB, ExbD, | | |
| and ExbB (gram- | | |
| negatives) | | |
| ipdC, iaaM | Auxin | |
| GA operon | Gibberelin | GA operon sources include those disclosed in Nett |
| containing core genes | | et al., 2020, doi: 10.1128/mSphere.00292-20 |
| cyp112, cyp114, | | |
| cyp117 (cytochrome | | |
| P450 (CYP) | | |
| monooxygenases), fd | | |
| (ferredoxin), sdr | | |
| (short-chain | | |
| dehydrogenase/ | | |
| reductase), ggps | | |
| (geranylgeranyl | | |
| diphosphate | | |
| synthase), cps and ks | | |
| (two diterpene | | |
| synthases/cyclases) | | |
| ACC deaminase, | ammonia | Down-regulation of ethylene by degradation of |
| AcdS | and α- | ethylene precursor 1-aminocyclopropane-1- |
| | ketobutyrate | carboxylate (ACC). AcdS sources include those in |
| | | Gao et al., 2020, doi: |
| | | 10.3390/microorganisms8010071 |
| Glyceraldehyde-3- | Volatile Organic | Production of 2,3-Butanediol. GAPDH and BDH |
| Phosphate | compounds | sources include those disclosed in Yang et al., |
| Dehydrogenase | (VOC) | 2013, doi: 10.1371/journal.pone.0076149 |
| (GAPDH) and 2,3- | | |
| butanediol | | |
| Dehydrogenase | | |
| (BDH) | | |
| OtsA, OtsB, and | Trehalose | TreS sources include those disclosed in Orozco- |
| TreS; TreS alone. | | Mosqueda, 2019, doi: 10.3389/fmicb.2019.01392. |
| gene encoding a | carbon- | CA gene sources include those disclosed in Chen |
| carbonic anhydrase | containing | et al., 2019, doi: 10.1007/s12088-018-0773-6; |
| (CA) (beta-CA or | compound | Clemente Capasso et al., 2012, doi: |
| alpha-CA) | (calcium | 10.3109/14756366.2012.703185 |
| | carbonate) | |

In certain embodiments, one or more genes set forth in Table 3 can be up-regulated by one or more of the phosphate-sensitive systems or genes set forth in Table 4. Particular embodiments of the systems for up-regulation of genes set forth in Table 3 are also disclosed within the following numbered embodiments 1-119 and 123.

TABLE 4
Non-limiting summary for gene up-regulation mechanisms
under decreased phosphate concentrations

| | | Non-limiting descriptions of |
|---|---|---|
| | Non-limiting materials | materials and methods which |
| | which can be used to | can be adapted for use in the |
| System | implement system | system |
| P-SP controlled | P-SP described in | GlnE-uAT described in: |
| expression of a | Table 5, SEQ ID NO: | US20210315212 incorporated herein by |
| GlnE protein | 417-428, 515, 517, or | reference in its entirety. |
| lacking an adenylyl | 518; GlnE-uAT | Schnabel and Sattely 2021, doi: |
| removing domain | proteins comprising a | 10.1128/AEM.00582-21 |
| which exhibits | protein sequence having | Schnabel and Sattely 2021, doi: |
| unidirectional | at least 70%, 80%, 85%, | 10.1021/acssynbio.1c00287 |
| adenyly ltransferase | 90%, 95%, 98%, or | |
| (uAT) activity | 99% identity to SEQ ID | |
| (GlnE-uAT) | NO: 16-24, or 25. | |
| P-SP controlled | P-SP described in Table | NifA described US20210315212 |
| expression of a NifA | 5, SEQ ID NO: 417- | incorporated herein by reference in its |
| protein | 428, 515, 517, or 518; | entirety. |
| P-SP controlled | P-SP described in Table | GlnR described US20210315212 |
| expression of a | 5, SEQ ID NO: 417- | incorporated herein by reference |
| GlnR protein | 428, 515, 517, or 518 | in its entirety. |
| P-SP controlled | Table 5 P-SP described | Glutaminase enzymes disclosed in |
| expression of a | in Table 5, SEQ ID NO: | US20210315212 incorporated |
| glutaminase enzyme | 417-428, 515, 517, or | herein by reference in its entirety. |
| | 518 | |
| P-SP controlled | Table SP-SP described | Nif gene clusters disclosed in |
| expression of a | in Table 5, SEQ ID NO: | US20210315212 incorporated |
| refactored nif or fix | 417-428, 515, 517, or | herein by reference in its entirety. |
| gene cluster | 518 | |
| P-SP controlled | P-SP described in Table | Hooshangi et al., 2004, doi: |
| expression of a | 5, SEQ ID NO: 417- | 10.1073pnas.0408507102;; |
| repressor which | 428, 515, 517, or 518; | Ramos et al., 2005, |
| controls a second | Lambda phage | doi.org/10.1128/mmbr.69.2.326- |
| gene encoding a | repressor (cI), the tet | 356.2005 |
| second repressor in | repressor (TetR), the lac | |
| a repressor cascade | repressor (LacI), and | |
| comprising an even | PhlF and respective | |
| number (2, 4, 6, 8, or | binding sites (e.g., in | |
| more) of repressors | Table 5) | |
| and repressed genes | | |
| encoding repressors, | | |
| where the last | | |
| repressor in the | | |
| cascade can repress | | |
| expression of the | | |
| gene targeted for up- | | |
| regulation when the | | |
| P-SP is inactive. | | |
| P-SP controlled | P-SP described in Table | Pinto et al., doi: |
| expression of a | 5, SEQ ID NO: 417- | 10.1093/nar/gky614 |
| transcriptional | 428, 515, 517, or 518; | |
| activator (TA) | Transcriptional | |
| which activates | activators including tet | |
| expression of the | responsive element- | |
| gene encoding the | binding tTA | |
| RNA or protein of | transcription factor or | |
| interest | an activator domain | |
| | fused to a DNA | |
| | targeting protein; TA | |
| | also include sigma | |
| | factors which bind the | |
| | core promoter and RNA | |
| | polymerase. | |
| P-SP controlled | P-SP described in Table | Pinto et al., doi: |
| expression of a | 5, SEQ ID NO: 417- | 10.1093/nar/gky614 |
| transcriptional | 428, 515, 517, or 518; | |
| activator (TA) | Transcriptional | |
| which controls a | activators including tet | |
| second gene | responsive element- | |
| encoding a second | binding tTA | |
| activator in a | transcription factor or | |
| transcriptional | an activator domain | |
| activator cascade | fused to a DNA | |
| comprising of | targeting protein; TA | |
| transcriptional | also include sigma | |
| activators and | factors which bind the | |
| activated genes | core promoter and RNA | |
| encoding activators | polymerase. | |
| where the last | | |
| activator in the | | |
| cascade can activate | | |
| expression of the | | |
| gene targeted for up- | | |
| regulation when the | | |
| P-SP is active. | | |
| P-SP controlled | P-SP described in Table | U.S. Pat. No. 10,614,353, incorporated |
| expression of a site- | 5, SEQ ID NO: 417- | herein by reference in its entirety. |
| specific | 428, 515, 517, or 518; | Siuti et al. 2013, doi: 10.1038/nbt.2510 |
| recombinase (SSR) | | Bernabé-Orts et al. 2020, |
| or integrase protein | | doi.org/10.1093/nar/gkaa104 |
| in a cell containing | | |
| an SSR- or | | |
| integrase- | | |
| controlled promoter | | |
| switch (promoter is | | |
| operably linked to | | |
| target gene(s) upon | | |
| inversion by the | | |
| SSR or integrase). | | |
| P-SP controlled | P-SP described in Table | Transcriptional activators |
| expression of a | 5, SEQ ID NO: 417- | disclosed in U.S. Pat. No. |
| transcriptional | 428, 515, 517, or 518; | 10,614,353, incorporated herein |
| activator protein | | by reference in its entirety. |
| which binds a | | |
| natural or synthetic | | |
| DNA motif in a | | |
| promoter of the | | |
| target gene | | |
| P-SP controlled | P-SP described in Table | U.S. Pat. No. 10,614,353, incorporated |
| expression of a site- | 5, SEQ ID NO: 417- | herein by reference in its entirety. |
| specific | 428, 515, 517, or 518; | Siuti et al. 2013, doi: 10.1038/nbt.2510 |
| recombinase (SSR) | Constitutive and | Bernabé-Orts et al. 2020, |
| or integrase protein | inducible promoters | doi.org/10.1093/nar/gkaa104 |
| in a cell containing | used in the SSR or | |
| an SSR- or | integrase controlled | |
| integrase- | promoter switch. | |
| controlled promoter | | |
| switch where the | | |
| promoter is | | |
| operably linked to a | | |
| first gene prior to | | |
| inversion by the | | |
| SSR or integrase | | |
| and is operably | | |
| linked to a up- | | |
| regulation gene | | |
| target upon | | |
| inversion. | | |

In certain embodiments, target genes encoding an RNA or protein of interest can be upregulated or down regulated by an SSR- or integrase-controlled promoter switch where a control element comprising a promoter can be inverted. Examples of such promoter switches include those in Tables 1-4 or the numbered embodiments. In certain embodiments, inversion of the promoter by the SSR- or integrase- can result in the down-regulation of the RNA sequence or protein of interest by disrupting operable linkage of the promoter to DNA encoding the RNA or protein. In certain embodiments, inversion of the promoter by the SSR- or integrase- can result in the up-regulation of the RNA sequence or protein of interest by operably linking the promoter to DNA encoding the RNA or protein (e.g., an SSR- or integrase-controlled promoter switch). Control elements used in the promoter switch can comprise a constitutive promoter, an inducible promoter, or phosphate-sensitive promoter and at least a segment of a 5′ UTR. In still other embodiments, an SSR- or integrase-controlled promoter switch can be designed to express a first gene before expression of the SSR or integrase and a second gene after expression of the SSR or integrase by the PS-P. In certain embodiments, the first gene can be selected from genes targeted for down-regulation in Table 1 and the second gene can be selected from a gene targeted for up-regulation in Table 3. In certain embodiments, the first gene can be a wild-type or even improved copy of a gene targeted for down-regulation in Table 1 and the second gene can be a wild-type copy of that same first gene from Table 1 which is expressed at levels lower than the wild-type or first gene, a mutated variant of that same first gene from Table 1 with reduced enzymatic or biological activity, or a mutated variant of that same first gene from Table 1 which is also expressed at levels lower than the wild-type or first gene.

In one embodiment, the SSR- or integrase-controlled promoter switch is placed between a first gene encoding a GlnA (glutamine synthetase (GS)) protein with wild-type or improved catalytic activity in comparison to wild-type GS and a second gene encoding: (i) a wild-type GS with reduced levels of expression in comparison to the wild-type glnA gene, (ii) a GS variant with decreased catalytic activity in comparison to wild-type GS, or (iii) a GS variant with decreased catalytic activity in comparison to wild-type GS and reduced levels of expression in comparison to the wild-type glnA gene, where the promoter in the SSR- or integrase-controlled promoter switch is operably linked to the first gene prior to SSR- or integrase-mediated promoter inversion resulting from PS-P promoter activation of the SSR or integrase. GEB comprising this promoter switch will express wild-type or catalytically improved GS prior to PS-P promoter-mediated activation of the SSR or integrase, promoting growth of the GEB in plant growth media. After PS-P promoter-mediated activation of the SSR or integrase, the promoter in the promoter switch is inverted and operably linked to the wild-type GS with reduced expression, variant GS with reduced catalytic activity, or variant GS with reduced catalytic activity and reduced expression, resulting in ammonia production. Reductions in expression of the second gene encoding a wild-type or GS variant can be achieved in a variety of ways including use of weak ribosome binding sites in the 5′ UTR, use of non-preferred codons, substitution of the ATG start codon with a GTG start codon, mutations that result in decrease protein or mRNA and/or protein stability, and combinations thereof. Certain methods of reducing expression of a glnA gene disclosed in US20200331820 can be adapted for use in embodiments disclosed herein.

In one embodiment, the SSR- or integrase-controlled promoter switch is placed between a first gene comprising a glnE gene encoding a wild-type GS adenylyltransferase protein and a second gene comprising a glnE gene encoding a GS adenylyltransferase protein lacking an adenylyl removing domain which exhibits unidirectional adenylyltransferase (uAT) activity (e.g., a GlnE-uAT gene), where the promoter in the SSR- or integrase-controlled promoter switch is operably linked to the first gene prior to SSR- or integrase-mediated promoter inversion resulting from PS-P promoter activation of the SSR or integrase. GEB comprising this promoter switch will express wild-type GlnE protein prior to PS-P promoter-mediated activation of the SSR or integrase, promoting growth of the GEB in plant growth media. After PS-P promoter-mediated activation of the SSR or integrase, the promoter in the promoter switch is inverted and operably linked to the GlnE-uAT gene, resulting in the adenylation and inactivation of GS and ammonia production. Sources of GlnE-uAT genes are set forth in Table 3.

In one embodiment, the SSR- or integrase-controlled promoter switch is placed between a first gene comprising a nifL gene and a second gene comprising a nifA gene, where the promoter in the SSR- or integrase-controlled promoter switch is operably linked to the first nifL gene prior to SSR- or integrase-mediated promoter inversion resulting from PS-P promoter activation of the SSR or integrase. GEB comprising this promoter switch will express wild-type NifL protein prior to PS-P promoter-mediated activation of the SSR or integrase, promoting growth of the GEB in plant growth media. After PS-P promoter-mediated activation of the SSR or integrase, the promoter in the promoter switch is inverted and operably linked to the nifA gene, resulting in the down-regulation of nifL, the up-regulation of nifA, and ammonia production.

In certain embodiments, it is desirable to delay expression of genes encoding an RNA or protein of interest (e.g., genes disclosed in Table 1 or 3) by constructing a PS-P controlled cascade of repressors/repressor-controlled genes encoding repressors and/or a PS-P controlled cascade of transcriptional activators/transcriptional activator-controlled genes encoding transcriptional activators in the GEB. In certain embodiments, a gene encoding the RNA or protein of interest or which regulates expression of the gene encoding the RNA or protein of interest is at the end of the cascade. At the head of the repressor and/or transcriptional-activator cascade is a PS-P promoter that drives expression of the first repressor or transcriptional activator, respectively. In certain embodiments, a repressor cascade can further comprise a gene encoding a transcriptional activator which is controlled by a repressor in the cascade (e.g., the terminal repressor in the cascade which regulates the RNA or protein of interest is at the end of the cascade). In certain embodiments, a transcriptional activator cascade can further comprise a gene encoding a repressor which is controlled by a transcriptional activator in the cascade (e.g., the terminal repressor in the cascade). In a repressor cascade, each repressor represses the subsequent repressor in the cascade. In a transcriptional activator cascade, each transcriptional activator activates the subsequent transcriptional activator in the cascade. Descriptions of such regulatory cascades which can be adapted for use in the PS-P controlled regulatory cascades described herein include regulatory cascades disclosed in Tables 2 and 4 as well as in those disclosed in Hooshangi et al., 2004, doi: 10.1073pnas.0408507102 and Pinto et al., doi: 10.1093/nar/gky614.

Without being limited by theory, it is believed that the delay in expression of the gene encoding the RNA or protein of interest is a factor of the number of repressor modules and/or transcriptional activator modules are in the cascade, the rate at which each of the repressors and/or transcriptional activator degrade and/or accumulate in the cell. It is further believed that delaying expression of RNAs or proteins of interest is beneficial insofar as expression of those RNAs, proteins, or the agriculturally relevant compounds they produce can in certain embodiments reduce the GEB's ability to compete against other microorganisms in the plant growth media and/or on the plant (e.g., root system). It is thus believed that such delays in expression can promote growth of the GEB to sufficient cell density in the plant growth media and/or plant before the agriculturally relevant compound is produced.

Bacteria selected from the groups of bacteria of the taxonomic classes of alphaproteobacteria, betaproteobacteria, and gammaproteobacteria can be modified to obtain the GEB disclosed herein. In certain embodiments, the bacteria selected for modification to obtain the GEB are bacteria in the taxonomic genera of Acetobacter, Acidothermus, Acinetobacter, Agrobacterium, Aromatoleum, Arthrobacter, Azoarcus, Azorhizobium, Azospirillum, Azotobacter, Bacillus, Bifidobacterium, Bradyrhizobium, Burkholderia, Conexibacter, Curtobacterium, Ensifer, Enterobacter, Erwinia, Escherichia, Flavobacterium, Frankia, Gaiella, Gluconacetobacter, Gluconobacter, Herbaspirillum, Klebsiella, Kosakonia, Lactobacillus, Lactococcus, Lysinibacillus, Maritimibacter, Methylobacterium, Nitrosocosmicus, Nitrososphaera, Paenarthrobacter, Paenibacillus, Panotea, Pediococcus, Peribacillus, Phytobacter, Priestia, Pseudarthrobacter, Pseudomonas, Rahnella, Rhizobium, Rhodococcus, Rhodoplanes, Rhodopseudomonas, Rhodospirillum, Serratia, Solirubrobacter, Sphingobacterium, Sphingomonas, Stenotrophomonas, Streptomyces, Stutzerimonas, Variovorax, Xanthobacter, and Yoonia, optionally wherein the bacteria are selected from at least one of the taxonomic genera selected from the group consisting of Azospirillum, Enterobacter, Herbaspirillum, Kosakonia, Klebsiella, Paenibacillus, Phytobacter, Pseudomonas, Rahnella, Sphingomonas, or Variovorax. In certain embodiments, the GEB and/or bacteria used to obtain the GEB are bacteria which have been modified and/or selected for increased potential to colonize the roots of target crop plants (e.g., maize, rice, wheat, and the like). Genes which have been modified for improved colonization include bcsll, bcslll, yjbE, fhaB, pehA, glgA, otsB, treZ, and cysZ genes (US Patent. Applic. Publ. No. 20210315212 and WO2019032926, both incorporated herein by reference in their entireties). In certain embodiments, the genetically engineered strains disclosed herein can be obtained by modification of Klebsiella, Kosakonia, or Rahnella strains, and include such strains deposited as NCMA 201701002, a bacterium deposited as NCMA 201708004, a bacterium deposited as NCMA 201708003, a bacterium deposited as NCMA 201708002, a bacterium deposited as NCMA 201712001, or a bacterium deposited as NCMA 201712002. These strains were deposited with the Bigelow National Center for Marine Algae and Microbiota (NCMA), located at 60 Bigelow Drive, East Boothbay, Me. 04544, USA under the terms of the Budapest Treaty as described in US Patent. Applic. Publ. No. 20210315212. In certain embodiments, the genetically engineered bacteria disclosed herein can be selected for improved plant colonization and/or improved production of nitrogenous compounds (e.g., ammonia) by various methods including those disclosed in US Patent Application No. 20240010576, incorporated herein by reference in its entirety. Examples of other bacteria that can be modified to obtain the GEB disclosed herein also include Rahnella aquatilis and Enterobacter sacchari strains were deposited with the American Type Culture Collection and assigned ATTC Patent Deposit Designation numbers PTA-122293 and PTA-122294, respectively, as described in US Patent Application No. 20240010576.

A variety of control elements comprising PS-P promoters and optionally 5′ UTR segments can be used in the GEB, methods, and systems provided herein. Desirable characteristics of such control elements include activation of expression of operably linked RNAs or proteins in agriculturally useful bacteria in response to decreases in phosphate concentration in the plant growth medium. Such agriculturally useful bacteria include bacteria capable of growth in plant growth media and in particular growth in plant growth media used to grow crop plants and/or growth on and/or in crop plants. In certain embodiments, the control elements comprising Pho boxes and/or PS-P promoters will be derived in whole or in part from agriculturally useful bacteria which include: (i) alphaproteobacteria, betaproteobacteria, and gammaproteobacteria; (ii) bacteria in the genus Acetobacter, Acidothermus, Acinetobacter, Agrobacterium, Aromatoleum, Arthrobacter, Azoarcus, Azorhizobium, Azospirillum, Azotobacter, Bacillus, Bifidobacterium, Bradyrhizobium, Burkholderia, Conexibacter, Curtobacterium, Ensifer, Enterobacter, Erwinia, Escherichia, Flavobacterium, Frankia, Gaiella, Gluconacetobacter, Gluconobacter, Herbaspirillum, Klebsiella, Kosakonia, Lactobacillus, Lactococcus, Lysinibacillus, Maritimibacter, Methylobacterium, Nitrosocosmicus, Nitrososphaera, Paenarthrobacter, Paenibacillus, Panotea, Pediococcus, Peribacillus, Phytobacter, Priestia, Pseudarthrobacter, Pseudomonas, Rahnella, Rhizobium, Rhodococcus, Rhodoplanes, Rhodopseudomonas, Rhodospirillum, Serratia, Solirubrobacter, Sphingobacterium, Sphingomonas, Stenotrophomonas, Streptomyces, Stutzerimonas, Variovorax, Xanthobacter, and Yoonia; (iii) Azospirillum, Enterobacter, Herbaspirillum, Kosakonia, Klebsiella, Paenibacillus, Phytobacter, Pseudomonas, Rahnella, Sphingomonas, or Variovorax. In certain embodiments, the control elements comprising the PS-P are used in bacteria of the genus or the species from which they were derived in whole or in part.

In certain embodiments, the control elements comprising PS-P promoters and optionally 5′ UTR segments will comprise “Pho box” elements or the corresponding PS-P promoters obtained from E. coli. Such E. coli Pho Box sequences can comprise those set forth in SEQ ID NO: 234-345, 378-382, and variants thereof comprising 1, 2, or 3 nucleotide substitutions. Such E. coli PS-P promoters can comprise DNA molecules having at least 85%, 90%, 95%, 98%, or 99% sequence identity across the entire length of any of SEQ ID NO: 419 and 420. In certain embodiments, the control elements comprising the E. coli Pho Box sequences provided herein can activate expression of operably linked RNAs or proteins in agriculturally useful bacteria including various gram-negative bacteria (e.g., Klebsiella, Escherichia, or Serratia) in response to decreases in phosphate concentration.

In certain embodiments, the control elements comprising PS-P promoters and optionally 5′ UTR segments will comprise “Pho box” elements or the corresponding PS-P promoters obtained from S. coelicolor. Such S. coelicolor Pho box sequences can comprise those set forth in SEQ ID NO: 346-375, and variants thereof comprising 1, 2, or 3 nucleotide substitutions. In certain embodiments, the control elements comprising the S. coelicolor Pho Box sequences provided herein can activate expression of operably linked RNAs or proteins in agriculturally useful bacteria including various gram-positive bacteria and actinomycetes (e.g., Streptomyces sp.) in response to decreases in phosphate concentration.

In certain embodiments, the control elements comprising PS-P promoters and optionally 5′ UTR segments will comprise “Pho box” elements (specific regulator-binding DNA sequences that facilitate the activation or inhibition of gene expression in a phosphate-sensitive manner) or the corresponding PS-P promoters are obtained in whole or in part from various agriculturally useful bacteria (AUB). AUB include bacteria in the taxonomic genera of Acetobacter, Acidothermus, Acinetobacter, Agrobacterium, Aromatoleum, Arthrobacter, Azoarcus, Azorhizobium, Azospirillum, Azotobacter, Bacillus, Bifidobacterium, Bradyrhizobium, Burkholderia, Conexibacter, Curtobacterium, Ensifer, Enterobacter, Erwinia, Escherichia, Flavobacterium, Frankia, Gaiella, Gluconacetobacter, Gluconobacter, Herbaspirillum, Klebsiella, Kosakonia, Lactobacillus, Lactococcus, Lysinibacillus, Maritimibacter, Methylobacterium, Nitrosocosmicus, Nitrososphaera, Paenarthrobacter, Paenibacillus, Panotea, Pediococcus, Peribacillus, Phytobacter, Priestia, Pseudarthrobacter, Pseudomonas, Rahnella, Rhizobium, Rhodococcus, Rhodoplanes, Rhodopseudomonas, Rhodospirillum, Serratia, Solirubrobacter, Sphingobacterium, Sphingomonas, Stenotrophomonas, Streptomyces, Stutzerimonas, Variovorax, Xanthobacter, and Yoonia. Such AUB Pho Box sequences can comprise those set forth in SEQ ID NO: 376, 377, 383-397, and 525-529, and variants thereof comprising 1, 2, or 3 nucleotide substitutions. Such AUB PS-P promoters can comprise DNA molecules having at least 85%, 90%, 95%, 98%, or 99% sequence identity across the entire length of any one of SEQ ID NO: 417-418, 421-428, 515, 517, and 518. Such Pho boxes and PS-P promoters can also include those which are operably linked to a phoA, phoX, phy, or pstS gene encoding a protein having at least 76%, 80%, 85%, 90%, 95%, 98%, or 99% sequence identity to a PhoA, PhoX, Phy (phytase), or PstS protein set forth in Table 5 and the sequence listing. In certain embodiments, the control elements comprising the AUB Pho box sequences and AUB PS-P promoters provided herein can activate expression of operably linked RNAs or proteins in agriculturally useful bacteria including Gluconacetobacter sp., Azorhizobium sp., Azospirillum sp., Herbaspirillum sp., Kosakonia sp., Paenibacillus sp., Pseudomonas sp. Enterobacter sp., Klebsiella sp., Paenibacillus sp., Phytobacter sp., Rahnella sp., Sphingomonas sp., or Variovorax sp. in response to decreases in phosphate concentration. In certain embodiments, the control elements comprising the AUB Pho box sequences and AUB PS-P promoters provided herein can activate expression of operably linked RNAs or proteins in: (i) a bacterium of the genus Azospirillum, Enterobacter, Herbaspirillum, Kosakonia, Klebsiella, Paenibacillus, Phytobacter, Pseudomonas, Rahnella, Sphingomonas, or Variovorax; and/or (ii) a Gluconacetobacter diazotrophicus, Azorhizobium caulinodans, Azospirillum brasilense, Herbaspirillum seropedicae, Kosakonia radicincitans, Paenibacillus azotofixans, and Pseudomonas stutzeri isolate, strain, or derivative thereof. In certain embodiments, a control element comprising an AUB Pho box sequence or AUB PS-P promoters derived in whole or in part from Gluconacetobacter sp., Azorhizobium sp., Azospirillum sp., Herbaspirillum sp., Kosakonia sp., Klebsiella sp., Paenibacillus sp., or Pseudomonas sp. (e.g., the corresponding AUB Pho box sequences and AUB PS-P promoters set forth in Table 5) are respectively used in a GEB obtained from: (i) a Gluconacetobacter sp., Azorhizobium sp., Azospirillum sp., Herbaspirillum sp., Kosakonia sp., Paenibacillus sp., or Pseudomonas sp.; and/or (ii) a bacterium of the genus Azospirillum, Enterobacter, Herbaspirillum, Kosakonia, Klebsiella, Paenibacillus, Phytobacter, Pseudomonas, Rahnella, Sphingomonas, or Variovorax. In certain embodiments, a control element comprising an AUB Pho box sequences or AUB PS-P promoter derived in whole or in part from Gluconacetobacter diazotrophicus, Azorhizobium caulinodans, Azospirillum brasilense, Herbaspirillum seropedicae, Kosakonia radicincitans, Klebsiella sp., Paenibacillus azotofixans, or Pseudomonas stutzeri (e.g., the corresponding AUB Pho box sequences and AUB PS-P promoters set forth in Table 5) are respectively used in a GEB obtained from: (i) a Gluconacetobacter diazotrophicus, Azorhizobium caulinodans, Azospirillum brasilense, Herbaspirillum seropedicae, Kosakonia radicincitans, Paenibacillus azotofixans, or Pseudomonas stutzeri isolate, strain, or derivative thereof; and/or (ii) a bacterium of the genus Azospirillum, Enterobacter, Herbaspirillum, Kosakonia, Klebsiella, Paenibacillus, Phytobacter, Pseudomonas, Rahnella, Sphingomonas, or Variovorax. In certain embodiments, the AUB Pho Box sequence can comprise a DNA molecule set forth in SEQ ID NO: 383-391, 397, 525-529, and variants thereof comprising 1, 2, or 3 nucleotide substitutions. In certain embodiments, the AUB Pho Box sequence or PS-P promoter can comprise a DNA molecule having at least 85%, 90%, 95%, 98%, or 99% sequence identity across the entire length of any one of SEQ ID NO: 417-418, 421-425, 428, 515, 517, or 518.

In certain embodiments, the heterologous gene expression cassette comprising the control elements comprising the Pho box sequences and PS-P promoters (e.g., the corresponding AUB Pho box sequences and AUB PS-P promoters set forth in Table 5) is integrated at a location in the chromosome of the genetically engineered bacterium which does not comprise the location of an endogenous phosphate-sensitive promoter in the unmodified agriculturally relevant bacterium. In certain embodiments, a control element comprising a phoA Pho box or phoA PS-P promoter (e.g., a phoA box or phoA PS-P promoter in Table 5) is integrated in the genome of the GEB at a location other than the location of the endogenous (e.g., wild-type) phoA box or phoA PS-P promoter in the GEB. In certain embodiments, a control element comprising a phoX Pho box or phoX PS-P promoter (e.g., a PhoX box or phoX PS-P promoter in Table 5) is integrated in the genome of the GEB at a location other than the location of the endogenous (e.g., wild-type) phoX box or phoX PS-P promoter in the GEB. In certain embodiments, a control element comprising a phy Pho box or phy PS-P promoter (e.g., a phy Pho box or phy PS-P promoter in Table 5) is integrated in the genome of the GEB at a location other than the location of the endogenous (e.g., wild-type) phy Pho box or phy PS-P promoter in the GEB. In certain embodiments, a control element comprising a pstS Pho box or pstS PS-P promoter (e.g., a pstS Pho box or pstS PS-P promoter in Table 5) is integrated in the genome of the GEB at a location other than the location of the endogenous (e.g., wild-type) pstS Pho box or pstS PS-P promoter in the GEB. Methods for inserting control elements comprising Pho boxes or PS-P promoters at sites distinct from the endogenous gene include Tn7 transposon mediated insertion (McKenzie and Craig, N. L., 2006, doi: 10.1186/1471-2180-6-39).

Methods of producing preparations of the genetically engineered bacterium (GEB) for use in treating plant growth media, plants, plant parts, plant propagules, and/or for formulating a composition comprising the GEB are provided herein. In certain embodiments, the methods can comprising growing the GEB in a bacterial growth medium comprising a carbon and nitrogen source and harvesting the GEB from the media. Conditions for growing the GEB include axenic growth in continuous stirred tank reactors, batch fermentation reactors, and the like. In certain embodiments, phosphate concentrations in the bacterial growth medium are kept at a level sufficient to suppress the expression of the phosphate-sensitive promoter(s) which drive expression of the protein or RNA of interest. Phosphate concentrations sufficient to suppress the expression of the phosphate-sensitive promoter(s) include phosphate concentrations of at least about 20 μM, 50 μM, or 100 μM. In certain embodiments, the GEB are grown in a bacterial growth media comprising phosphate concentrations of at least 1 mM, 5 mM, 10 mM, or 20 mM. In certain embodiments, the GEB are grown in a bacterial growth media comprising phosphate concentrations of about 1 mM, 5 mM or 10 mM to about 30 mM, 50 mM, or 100 mM.

Compositions comprising one or more of the genetically engineered bacterium (GEB) are also provided herein. Such compositions can be adapted for storage of the GEB and/or for use of the GEB in the methods and agricultural systems disclosed herein. In certain embodiments, the compositions comprising the GEB can be used to treat a plant part including a leaf, stem, root, and/or seed by least partially coating the plant part with the composition. In certain embodiments, the compositions comprising the GEB can placed in plant growth medium (e.g., soil and/or water) prior to, during, and/or after depositing or establishing a seed, a seedling, plant, or vegetative propagule in the plant growth medium. In certain embodiments, a composition comprising the GEB is in a solid form. Such solid compositions can include those comprising a wettable powder, granules, a gel, pellets, or microencapsulated particles. In certain embodiments, a composition comprising the GEB is in a liquid form. Such liquid compositions can include those comprising an aqueous solution, aqueous suspension, water-in-oil emulsion, an oil, or an alcohol. Descriptions of compositions and components thereof which can be adapted for use with the GEB, related methods, agricultural systems, seedlings, plant parts, and vegetative propagules provided herein include those described below and/or in US Patent Application Publication Nos. US20210315212A1 and 20230148607, which are incorporated herein by reference in their entirety.

Compositions comprising the GEB provided herein can comprise an agriculturally acceptable carrier. Such carriers include liquid carriers comprising water, aqueous solutions, plant oils, and combinations thereof. Such carriers can in other embodiments comprise one of more solids including diatomaceous earth, loam, silica, clay, bentonite, vermiculite, seed cases, plant products (e.g., ground hulls, husk, stalks, stems, leaves, and the like), animal products, or combinations thereof.

Compositions comprising the GEB provided herein can comprise an agriculturally acceptable adjuvant.

In certain embodiments, the agriculturally acceptable adjuvant is an adhesive agent (e.g., an agent which promotes adherence of the composition and/or GEB to a plant part). In certain embodiments, the adhesive agents comprise one or more alginates, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and/or polyoxyethylene-polyoxybutylene block copolymers. In certain embodiments, the adhesive agents can comprise one or more waxes (e.g., carnauba wax, beeswax, or Chinese wax, shellac wax, spermaceti wax, candelilla wax, castor wax, ouricury wax, or rice bran wax), a polysaccharide (e.g., starch, dextrins, maltodextrins, alginate, and chitosans), a fat, oil, a protein (e.g., gelatin and zeins), gum ambles, and/or a shellac. In certain embodiments, adhesive agents can comprise one or more polymers or copolymers including polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and/or polychloroprene.

In certain embodiments, the agriculturally acceptable adjuvant is a desiccant or mixture thereof. In certain embodiments, the desiccant comprises one or more of trehalose, sucrose, glycerol, and/or methylene glycol. In certain embodiments, the desiccant comprises one or more non-reducing sugars and sugar alcohols (e.g., mannitol or sorbitol). In certain embodiments, desiccants are provided at about 5% to about 50% by weight/volume (w/v), about 10% to about 40% (w/v), about 15% to about 35% (w/v), or about 20% to about 30% (w/v) in the composition.

In certain embodiments, the agriculturally acceptable adjuvant is a dispersant (e.g., a surfactant). In certain embodiments, the dispersant comprises one or more of a nitrogen-surfactant blend (e.g., Prefer 28 (Cenex), Surf-N(US), Inhance (Brandt), P-28 (Wilfarm) and Patrol (Helena)), an esterified seed oil (e.g., Sun-It II (AmCy), MSO (UAP), Scoil (Agsco), Hasten (Wilfarm) and Mes-100 (Drexel)), and/or an organo-silicone surfactant (e.g., Silwet L77 (UAP), Silikin (Terra), Dyne-Amic (Helena), Kinetic (Helena), Sylgard 309 (Wilbur-Ellis) and Century (Precision)). In one embodiment, the surfactant or mixture of surfactants are at a concentration of about 0.01% volume/volume (v/v) to about 10% v/v in the composition. In another embodiment, the surfactant or mixture of surfactants are at a concentration of 0.1% (v/v) to 1% (v/v) in the composition.

In certain embodiments, the agriculturally acceptable adjuvant is a fungicide, insecticide, a nematicide, a rodenticide, and/or a bacteriocide. When the adjuvant is a bacteriocide, it is a bacteriocide selected for compatibility with the GEB and/or provided at a concentration or in a form which does not compromise the viability of the GEB.

### NUMBERED EMBODIMENTS

Additionally, the following numbered embodiments are included in the disclosure.

1. A method of providing at least one agriculturally relevant compound to a plant comprising placing at least one genetically engineered bacterium into a plant growth medium, wherein the genetically engineered bacterium comprises a heterologous gene expression cassette comprising at least one nucleic acid sequence coding for at least one RNA sequence or protein of interest, wherein the heterologous gene expression cassette is operably linked to a control element comprising a phosphate-sensitive promoter, wherein said at least one RNA sequence or protein of interest is or causes the production of said at least one agriculturally relevant compound when a decrease in phosphate concentration in the plant growth medium activates expression of said at least one RNA sequence or protein of interest.

2. The method of embodiment 1, wherein the plant growth medium comprises soil and/or water, optionally wherein the soil and/or water is non-axenic.

3. The method of embodiment 1 or 2, wherein the placing is prior to, during, and/or after depositing a seed in the plant growth medium.

4. The method of embodiment 1 or 2, wherein the placing is prior to, during, and/or after depositing a vegetative propagule in the plant growth medium.

5. The method of embodiment 1, 2, or 3, wherein the placing comprises depositing a seed which is at least partially coated with the genetically engineered bacterium in the plant growth medium or depositing both the seed and a composition comprising the genetically engineered bacterium in the plant growth medium.

6. The method of embodiment 1, 2, or 3, wherein the placing comprises depositing the seed in furrow and contacting the seed in the furrow with a composition comprising the genetically engineered bacterium.

7. The method of any one of embodiments 1-6, wherein the placing of the genetically engineered bacterium in the plant growth medium is prior to, during, and/or after establishment of a plant in the plant growth medium.

8. The method of any one of embodiments 1-7, wherein the control element and/or the heterologous gene expression cassette further comprises at least a segment of a 5′ untranslated region (5′ UTR) which is operably linked to the phosphate-sensitive promoter and/or the heterologous gene expression cassette, optionally wherein the 5′ UTR comprises a 5′ UTR sequence which has at least 85%, 90%, 95%, 98%, or 99% sequence identity to an endogenous 5′ UTR sequence which is operably linked to an endogenous phosphate-sensitive promoter.

9. The method of embodiment 8, wherein the phosphate-sensitive promoter and/or the segment of the 5′ UTR which is operably linked to the phosphate-sensitive promoter comprises at least one copy of an operably linked Pho box, wherein the Pho box comprises the sequence of SEQ ID NO: 234-397, 525-528, or 529, and sequences with at least 95%, 98%, or 99% identity to any one of SEQ ID NO: 234-397, 525-528, or 529, optionally wherein the Pho box comprises SEQ ID NO: 376, 377, 383-397, 525-528, or 529.

10. The method of embodiment 8 or 9, wherein the phosphate-sensitive promoter and/or the segment of the 5′ UTR is a promoter and/or 5′ UTR derived from at least one gram-negative bacterium.

11. The method of embodiment 8, 9, or 10, wherein the phosphate-sensitive promoter and/or the segment of the 5′ UTR is a promoter and/or 5′ UTR derived from at least one bacterium of the taxonomic classes of alphaproteobacteria, betaproteobacteria, and gammaproteobacteria.

12. The method of any one of embodiments 8-11, wherein the phosphate-sensitive promoter and/or the segment of the 5′ UTR is a promoter and/or segment of a 5′ UTR derived from at least one bacterium of a genus selected from the group consisting of Acetobacter, Acidothermus, Acinetobacter, Agrobacterium, Aromatoleum, Arthrobacter, Azoarcus, Azorhizobium, Azospirillum, Azotobacter, Bacillus, Bifidobacterium, Bradyrhizobium, Burkholderia, Conexibacter, Curtobacterium, Ensifer, Enterobacter, Erwinia, Escherichia, Flavobacterium, Frankia, Gaiella, Gluconacetobacter, Gluconobacter, Herbaspirillum, Klebsiella, Kosakonia, Lactobacillus, Lactococcus, Lysinibacillus, Maritimibacter, Methylobacterium, Nitrosocosmicus, Nitrososphaera, Paenarthrobacter, Paenibacillus, Panotea, Pediococcus, Peribacillus, Phytobacter, Priestia, Pseudarthrobacter, Pseudomonas, Rahnella, Rhizobium, Rhodococcus, Rhodoplanes, Rhodopseudomonas, Rhodospirillum, Serratia, Solirubrobacter, Sphingobacterium, Sphingomonas, Stenotrophomonas, Streptomyces, Stutzerimonas, Variovorax, Xanthobacter, and Yoonia, optionally wherein the phosphate-sensitive promoter and/or the segment of the 5′UTR is derived from at least one bacterium of a genus selected from the group consisting of Azospirillum, Enterobacter, Herbaspirillum, Paenibacillus, Phytobacter, Pseudomonas, Klebsiella, Rahnella, Kosakonia, Sphingomonas, and Variovorax.

13. The method of any one of embodiments 8-12, wherein the phosphate-sensitive promoter and/or the segment of the 5′ UTR comprises a promoter and/or a segment of a 5′UTR of a phoA, phoX, phy, pstS gene, a variant thereof, or a combination thereof.

14. The method of any one of embodiments 8-13, wherein the phosphate-sensitive promoter and/or the segment of the 5′ UTR comprises a promoter and/or a segment of a 5′UTR of a promoter comprising the DNA sequence of SEQ ID NO: 417-428, 515, 516, or 518, a phosphate-sensitive promoter element of a promoter comprising the DNA sequence of SEQ ID NO: 417-428, 515, 516, 517, 518, a variant thereof, or a combination thereof, optionally wherein the variant has at least 76%, 80%, 85%, 90%, 95%, 98%, or 99% sequence identity to SEQ ID NO: 417-428, 515, 516, or 518.

15. The method of any one of embodiments 8-14, wherein the phosphate-sensitive promoter and/or the segment of the 5′ UTR comprises a promoter of a gene encoding:
i. a PhoA protein comprising a protein sequence selected from the group consisting of SEQ ID NO: 398, 400, 402, 404, and 406 or having an identity of at least 80%, 85%, 90%, 95%, 98%, or 99% with any one of SEQ ID NO: 398, 400, 402, 404, or 406;
ii. a PhoX protein comprising a protein sequence selected from the group consisting of SEQ ID NO: 408, 410, 411, 412, and 413 or having an identity of at least 80%, 85%, 90%, 95%, 98%, or 99% with any one of SEQ ID NO: 408, 410, 411, 412, and 413;
iii. a Phy protein comprising a protein sequence selected from the group consisting of SEQ ID NO: 414, 57-138, and 139 or having an identity of at least 80%, 85%, 90%, 95%, 98%, or 99% with any one of SEQ ID NO: 414, 57-138, or 139; and/or
iv. a PstS protein comprising a protein sequence selected from the group consisting of SEQ ID NO: 399, 401, 403, 405, 407, 409, 415, 416, 513, 523, and 524 or having an identity of at least 76%, 80%, 85%, 90%, 95%, 98%, or 99% with any one of SEQ ID NO: 399, 401, 403, 405, 407, 409, 415, 416, 513, 523, or 524.

16. The method of any one of embodiments 8-15, wherein the promoter region and/or the segment of the 5′ UTR comprises at least 50 nucleotides of DNA located upstream of a start codon of a gene encoding a PhoA, PhoX, Phy, or PstS protein, optionally wherein the promoter region and/or the segment of the 5′ UTR comprises about 50 to about 250 nucleotides of DNA located upstream of said start codon of said gene encoding said PhoA, PhoX, Phy, or PstS protein.

17. The method of embodiment 8, wherein the phosphate-sensitive promoter and/or the segment of the 5′ UTR comprises:
i. a phoA promoter comprising the DNA sequence of SEQ ID NO: 419;
ii. a phoX promoter comprising the DNA sequence of SEQ ID NO: 426;
iii. a phy promoter comprising the DNA sequence of SEQ ID NO: 427;
iv. a pstS promoter comprising a DNA sequence selected from the group consisting of SEQ ID NO: 417, 418, 420, 421, 422, 423, 424, 425, 428, 515, 517, and 518; and/or
v. a Pliar53 promoter comprising the DNA sequence of SEQ ID NO: 459;
vi. a variant of the promoters under i. to v. having an identity of at least 76%, 80%, 90%, 95%, 98%, or 99% with any of SEQ ID NO: 417, 418-428, 459, 515, 517, or 518, wherein said variant comprises at least one Pho box and/or retains at least one Pho box present in each promoter and wherein said variant promoters are activated by a decrease in phosphate concentration to about 0 μM to about 1 μM, 2 μM, 5 μM, 7 μM, 10 μM, 20 μM, 30 μM, 40 μM, or 50 μM.

18. The method of any one of embodiments 8-17, wherein the phosphate-sensitive promoter and/or the segment of the 5′ UTR or its variant is derived from the same genus or species as the genetically engineered bacterium.

19. The method of any one of embodiments 1-18, wherein the heterologous gene expression cassette is integrated at a location in the chromosome of the genetically engineered bacterium which does not comprise the location of an endogenous phosphate-sensitive promoter.

20. The method of any one of embodiments 1-19, wherein the heterologous gene expression cassette comprises one or more elements comprising:
a. a ribosome binding site (RBS), wherein the RBS is operably linked to the nucleic acid sequence coding for the protein of interest and optionally wherein the RBS is an RBS having at least 95% sequence identity to SEQ ID NO: 429-454, or 455; and/or
b. a terminator sequence (TS), wherein the TS is operably linked to the nucleic acid sequence coding for the RNA sequence or protein of interest and optionally wherein the TS is a TS having at least 95% sequence identity to SEQ ID NO: 456, 457, or 458.

21. The method of any one of embodiments 1 to 20, wherein the at least one agriculturally relevant compound is selected from the group consisting of:
a. at least one fertilizer or plant nutrient, optionally wherein the fertilizer or plant nutrient is selected from the group consisting of ammonia, ammonium, bioavailable carbon, calcium, iron, nitrate, nitrite, nitrogen, potassium, phosphate, sulfur, urea, zinc, a combination thereof, and a mixture thereof;
b. at least one pesticide, optionally wherein the pesticide is an RNA- and/or protein-based fungicide, insecticide, nematicide, antibacterial agent, and/or antiviral agent;
c. at least one phytohormone or plant growth regulator, optionally wherein the phytohormone or plant growth regulator is an auxin, a cytokinin, a gibberellin, abscisic acid, a brassinosteroid, jasmonic acid, a polyamine, a strigolactone, trehalose, and/or a volatile organic compound, or optionally wherein the phytohormone or plant growth regulator is cytokinin, indole butyric acid, and/or combinations thereof; and
d. at least one carbon-containing compound, optionally wherein the carbon-containing compound is bicarbonate, carbonate, CaCO₃, MgCO₃, CaMg(CO₃)₂, polyhydroxybutyrate, melanin, chitin, and/or combinations thereof.

22. The method of any one of embodiments 1-21, wherein the RNA sequence or protein of interest encoded by the heterologous gene expression cassette and operably linked to the control element comprises:
(a) a GlnE protein lacking an adenylyl removing domain which exhibits unidirectional adenylyltransferase (uAT) activity and the fertilizer is ammonia, optionally wherein the GlnE protein lacking an adenylyl removing domain comprises a protein sequence having at least 70%, 80%, 85%, 90%, 95%, 98%, or 99% identity to SEQ ID NO: 16-24, or 25 or optionally wherein the gene encoding the GlnE protein lacking an adenylyl removing domain comprises a DNA sequence having at least 70%, 80%, 85%, 90%, 95%, 98%, or 99% identity to SEQ ID NO: 1-14, or 15;
(b) a NifA protein, wherein the genetically engineered bacterium optionally comprises a loss-of-function mutation in any one or more of the amtB, draT, glnA, glnB, glnK, glnR, glnZ, or nifL genes, wherein the genetically engineered bacterium optionally comprises one or more heterologous genes from a wild-type or refactored nif or fix gene cluster and the fertilizer is ammonia;
(c) a GlnR protein and the fertilizer is ammonia;
(d) a glutaminase enzyme and the fertilizer is ammonia;
(e) a protein product of a refactored nif or fix gene cluster and the fertilizer is ammonia;
(f) a repressor protein, wherein the repressor protein binds a natural or synthetic DNA motif in the promoter of any one or more first target gene(s) of the genetically engineered bacterium and inhibits expression of any one or more of the protein products of the first target genes and/or wherein the repressor protein optionally comprises the lambda repressor (cI), the tet repressor (TetR), the lac repressor (LacI), a catalytically inactive RNA-guided DNA binding protein, a protein comprising a DNA-binding zinc finger domain, a transcription activator-like effector (TALE), or any variant thereof and/or optionally wherein the first target gene(s) is/are a glnA, amtB, glnB, glnK, glnZ, nifL, and/or draT gene and the fertilizer is ammonia;
(g) a non-coding synthetic small RNA (sRNA); optionally wherein the non-coding synthetic small RNA (sRNA) binds a natural or synthetic DNA and/or RNA motif in the promoter, 5′ UTR, and/or coding region of any one or more first target gene(s) of the genetically engineered bacterium, optionally wherein the non-coding synthetic small RNA (sRNA) comprises a guide RNA that additionally binds an RNA-guided DNA or RNA endonuclease or variant thereof and/or optionally wherein the first target gene(s) is/are a glnA, amtB, glnB, glnK, glnZ, nifL, and/or draT gene and the fertilizer is ammonia;
(h) a site-specific recombinase (SSR) or integrase protein: wherein any one or more first target gene(s) of the genetically engineered bacterium and/or a promoter operatively linked thereto are flanked by site-specific recombinase recognition sites (SSRRS) in a direct configuration, optionally wherein the SSRRS comprise attL and attR sites and the genetically engineered bacterium comprises a gene encoding a recombinase directionality factor (RDF) and/or optionally wherein the first target gene(s) is/are a glnA, amtB, glnB, segment of glnE encoding an adenylyl-removing domain of a glutamine synthetase adenylyltransferase, glnK, glnZ, nifL, and/or draT gene and the fertilizer is ammonia;
(i) a site-specific recombinase (SSR) or integrase protein: wherein any one or more first target gene(s), the promoter(s) thereof, and/or the 5′ UTR(s) thereof of the genetically engineered bacterium comprise(s) one or more internal synthetic SSRRS and wherein the gene(s), promoter(s), the 5′ UTR(s), and/or a segment(s) thereof is/are excised or inactivated after a recombination event; optionally wherein the genetically engineered bacterium further comprises a plasmid comprising an SSRRS; optionally wherein the genetically engineered bacterium further comprises one or more genes encoding a recombinase directionality factor (RDF); and/or optionally wherein the first target gene(s) is/are a glnA, amtB, glnB, glnE, glnK, glnZ, nifL, and/or draT gene and the fertilizer is ammonia;
(j) a site-specific recombinase (SSR) or integrase protein, wherein any one or more first target gene(s), the promoter(s) thereof, and/or the 5′ UTR(s) thereof of the genetically engineered bacterium comprise(s) one or more internal integrative SSRRS and retain(s) activity of said first target gene(s), promoter(s), and/or 5′ UTR(s) and wherein the genetically engineered bacterium further comprises an integrative element comprising an SSRRS; optionally wherein the internal integrative SSRRS is/are an attB site(s) and the integrative element comprises an SSRRS comprising an attP site; and/or optionally wherein the target gene(s) is/are glnA, amtB, glnB, glnK, glnZ, nifL, and/or draT and the fertilizer is ammonia;
(k) a site-specific recombinase (SSR) or integrase protein, wherein a control element comprising a promoter and/or at least a segment of a 5′ UTR: (i) is flanked by site-specific recombinase recognition sites (SSRRS) in an inverted configuration; and (ii) is operably linked to one or more first target gene(s) of the genetically engineered bacterium; optionally wherein the control element which is operably linked to said first target gene(s) comprises a constitutive promoter, an inducible promoter, or phosphate sensitive promoter; and/or optionally wherein the first target gene(s) is/are glnA, amtB, glnB, glnK, glnZ, nifL, and/or draT and the fertilizer is ammonia;
(l) a site-specific recombinase (SSR) or integrase protein, wherein a control element comprising a promoter and at least a segment of a 5′ UTR: (i) is flanked by site-specific recombinase recognition sites (SSRRS) in an inverted configuration; and (ii) is operably linked to one or more first target gene(s) of the genetically engineered bacterium upon inversion by the SSR or integrase; optionally wherein the control element which is operably linked to said first target gene(s) upon inversion by the SSR or integrase comprises a constitutive promoter, an inducible promoter, or phosphate-sensitive promoter; and/or optionally wherein the first target gene(s) is/are: (a) nfA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase, and the agriculturally relevant compound is phosphate;
(m) a site-specific DNA endonuclease, wherein any one or more first target gene(s) of the genetically engineered bacterium comprise(s) one or more specific DNA sequence(s) recognized by the site-specific DNA endonuclease, optionally wherein the site-specific DNA endonuclease comprises an RNA-guided DNA endonuclease, a protein comprising a DNA-binding zinc finger domain, a transcription activator-like effector (TALE), a meganuclease, a homing endonuclease, or a restriction endonuclease, and/or optionally wherein the first target gene(s) is/are glnA, amtB, glnB, glnK, glnZ, nifL, and/or draT and the fertilizer is ammonia;
(n) a protease which cleaves a protease specific recognition sequence (PSRS), wherein any one or more first target gene(s) of the genetically engineered bacterium comprise(s) one or more in-frame insertion(s) comprising DNA encoding the PSRS in the protein coding region of the first target gene(s), wherein the target protein product comprising the one or more in-frame insertion(s) has activity, and wherein cleavage of the target protein product(s) by the protease deactivates the target protein product(s), optionally wherein the location of the one or more in-frame insertion is given by Table 6, optionally wherein the first target gene(s) is/are under the control of a constitutive promoter, and/or optionally wherein the first target gene(s) is/are glnA, amtB, glnB, glnK, glnZ, nifL, and/or draT and the fertilizer is ammonia;
(o) a first protein of interest comprising a ClpAP ATP-dependent protease and a second protein of interest comprising a ClpS Leu/N-recognin, wherein any one or more first target gene(s) of the genetically engineered bacterium encodes a protein comprising an N-terminal-Leu, -Phe, -Trp, or -Tyr residue, and optionally wherein the first and second protein of interest are operably linked to distinct control elements, and/or optionally wherein the first target gene(s) is/are glnA, amtB, glnB, glnK, glnZ, nifL, and/or draT and the fertilizer is ammonia;
(p) a protease which cleaves a protease specific recognition sequence (PSRS), wherein any one or more first target gene(s) of the genetically engineered bacterium comprise(s) an in-frame insertion(s) of DNA encoding the PSRS at the N-terminus of the protein coding region of the gene followed by a -Leu, -Phe, -Trp, or -Tyr residue and wherein cleavage of PSRS from the N-terminus of the protein(s) encoded by the gene(s) by the protease results in a protein comprising an N-terminal-Leu, -Phe, -Trp, or -Tyr residue which is degraded by native ClpS and ClpAP, optionally wherein the first target gene(s) is/are glnA, amtB, glnB, glnK, glnZ, nifL, and/or draT and the fertilizer is ammonia;
(q) a transcriptional activator protein, wherein the transcriptional activator protein binds a natural or synthetic DNA motif in a promoter of a heterologous protease gene of the genetically engineered bacterium and activates expression of the protease, wherein any one or more first target gene(s) of the genetically engineered bacterium comprise(s) one or more in-frame insertion(s) of DNA encoding a PSRS, optionally wherein the transcriptional activator protein comprises a tet responsive element-binding tTA transcription factor or a transcriptional activator domain fused to a DNA targeting protein, optionally wherein the DNA targeting protein is a catalytically inactive RNA-guided DNA binding protein, a protein comprising a DNA-binding zinc finger domain, a transcription activator-like effector (TALE), or any variant thereof, optionally wherein the transcriptional activator domain is VP16, and/or optionally wherein the first target gene(s) is/are: glnA, amtB, glnB, glnK, glnZ, nifL, and/or draT and the fertilizer is ammonia;
(r) a repressor protein, wherein the repressor protein binds a natural or synthetic DNA motif in a promoter of a heterologous protease gene of the genetically engineered bacterium and inhibits expression of the protease, wherein any one or more first target gene(s) of the genetically engineered bacterium comprise(s) an in-frame insertion of DNA encoding an N-terminal amino acid tag designed to render the protein product(s) of the first target gene(s) inactive, followed by a PSRS, optionally wherein the repressor protein comprises the lambda repressor (cI), the tet repressor (TetR), the lac repressor (LacI), a catalytically inactive RNA-guided DNA binding protein, a protein comprising a DNA-binding zinc finger domain, a transcription activator-like effector (TALE), or any variant thereof, and/or optionally wherein the first target gene(s) is/are glnA, amtB, glnB, glnK, glnZ, nifL, and/or draT and the fertilizer is ammonia;
(s) a nanobody, wherein the nanobody binds a protein product of any one or more first target gene(s) of the genetically engineered bacterium, wherein binding of the nanobody to such protein product inhibits function of the protein product, optionally wherein the first target gene(s) is/are glnA, amtB, glnB, glnK, glnZ, nifL, and/or draT and the fertilizer is ammonia;
(t) an aptamer, wherein the aptamer binds a protein product of any one or more first target gene(s) of the genetically engineered bacterium and inhibits function of any one or more of the protein product(s) of the first target gene(s), optionally wherein the first target gene(s) is/are glnA, amtB, glnB, glnK, glnZ, nifL, and/or draT and the fertilizer is ammonia; or
(u) a transcriptional activator protein, wherein the transcriptional activator protein binds a natural or synthetic DNA motif in a promoter of any one or more first target gene(s) and increases expression of any one or more of the first target gene(s), optionally wherein the transcriptional activator protein comprises the tet responsive element-binding tTA transcription factor or a transcriptional activator domain fused to a DNA targeting protein, wherein the DNA targeting protein is optionally a catalytically inactive RNA-guided DNA binding protein, a protein comprising a DNA-binding zinc finger domain, a transcription activator-like effector (TALE), or any variant thereof, wherein the transcriptional activator domain is optionally VP16 and/or optionally wherein the first target gene(s) is/are any one or more of: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, and/or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase and the agriculturally relevant compound is phosphate.

23. The method of embodiment 22, wherein the integrase is a serine integrase, optionally wherein the serine integrase is a phage PhiC31 serine integrase, IntS, IntM, IntG—ICEMcSym 1271, YdcL—ICEBs, or Int—ICE SXT/R39 integrase, and the SSRRS are attB and attP sites recognized respectively by the PhiC31, IntS, IntM, IntG, YdcL, or Sxt/R39 integrase.

24. The method of embodiment 22, wherein: (i) one or more SSR is a yeast flippase (FLP) recombinase and the SSRRS are FRT sites; or (ii) one or more SSR is a Cre-recombinase and the SSRRS are loxP sites.

25. The method of embodiment 22, wherein the PSRS is inserted in a glutamine synthetase (GS) polypeptide between any one or more of the pairs of amino acid residues corresponding to:
(i) amino acid residues 98 and 99, 121 and 122, 279 and 280, and/or 285 and 286 of the glutamine synthetase (GS) polypeptide of SEQ ID NO: 229;
(ii) amino acid residues 98 and 99, 119 and 120, 283 and 284, and/or 298 and 299 of the glutamine synthetase (GS) polypeptide of SEQ ID NO: 230;
(iii) amino acid residues 448 and 449, 480 and 481, 505 and 506, 528 and 529, and/or 624 and 625 of the GlnE polypeptide of SEQ ID NO: 231;
(iv) amino acid residues 527 and 528, 537 and 538, and/or 547 and 548 of the GlnE polypeptide of SEQ ID NO: 232; or
(v) amino acid residues 422 and 423 and/or 608 and 609 of the GlnE polypeptide of SEQ ID NO: 233.

26. The method of embodiment 22 or 25, wherein the protease or heterologous protease:
a. comprises a tobacco etch virus (TEV) protease and the PSRS comprises the peptide EXXYXQ-(S/G) or ENLYFQ-(S/G/A/M/C/H), wherein X is any amino acid and the TEV protease cleaves between the Q and the S, G, A, M, C, or H residues;
b. comprises a tobacco vein mottling virus (TVMV) protease and the PSRS comprises the peptide ETVRFQ-(G/S), wherein the TVMV protease cleaves between the Q and S or G residues;
c. comprises a sunflower mild mosaic virus (SMMV) protease and the PSRS comprises the peptide EEIHLQ-(S/G), wherein the SMMV protease cleaves between the Q and S or G residues;
d. comprises a turnip mosaic virus (TrMV) protease and the PSRS comprises the peptide VXHQ or VRHQ-S, wherein X is any amino acid and the TrMV protease cleaves C-terminal to the Q residue;
e. comprises a soybean mosaic virus (SMV) protease and the PSRS comprises the peptide XVXXQ-(G/S), wherein X is any amino acid and the SMV protease cleaves between Q and S or G residues;
f. comprises a plum pox virus (PPV) protease and the PSRS comprises the peptide NVVVHQ-A, wherein the PPV protease cleaves between the Q and the A residue;
g. comprises a hepatitis C virus (HCV) NS3 protease and the PSRS comprises the peptide (D/E)XXXXC(A/S), wherein X is any amino acid and the HCV protease cleaves between the C and the A or S residues;
h. comprises an enterokinase and the PSRS comprises the peptide DDDDK, wherein the enterokinase cleaves C-terminal to the K residue;
i. comprises a Factor Xa protease and the PSRS comprises the peptide I(D/E)GR, wherein the Factor Xa protease cleaves C-terminal to the R residue; or
j. comprises a furin protease and the PSRS comprises the peptide RX(K/R)R, wherein the furin protease cleaves C-terminal to the C-terminal R residue.

27. The method of embodiment 21 or 22, wherein:
(i) the plant nutrient is zinc or potassium and the protein of interest or protein encoded by the target gene is a gluconate dehydrogenase (GAD) enzyme;
(ii) the plant nutrient is iron and the protein of interest or protein encoded by the target gene is a siderophore biosynthetic and transport protein optionally selected from a dhbACDEBF gene cluster; a non-ribosomal peptide synthetase (NRPS), polyketide synthase (PKS), and NRPS-independent siderophore synthetase (NIS); and major facilitator superfamily (MFS) transporters (ymfE), TonB, ExbD, and/or ExbB;
(iii) the phytohormone is auxin and the protein of interest or protein encoded by the target gene is an IpdC or IaaM protein;
(iv) the plant nutrient is ammonia and α-ketobutyrate and the protein of interest or protein encoded by the target gene is ACC deaminase;
(v) the plant growth regulator is a volatile organic compound and the proteins of interest or proteins encoded by the target genes are Glyceraldehyde-3-Phosphate Dehydrogenase (GAPDH) and 2,3-butanediol Dehydrogenase (BDH);
(vi) the agriculturally relevant compound is trehalose and the protein(s) of interest or proteins encoded by the target gene are OtsA, OtsB, and TreS or TreS; or
(vii) the carbon containing compound is calcium carbonate and the protein of interest is a beta-carbonic anhydrase or alpha-carbonic anhydrase.

28. The method of any one of embodiments 1-22, wherein the protein of interest comprises a site-specific recombinase (SSR) or integrase protein, wherein a control element comprising a promoter and/or at least a segment of a 5′ UTR: (i) is flanked by site-specific recombinase recognition sites (SSRRS) in an inverted configuration; (ii) is operably linked to one or more first target gene(s) of the genetically engineered bacterium; and (iii) is operably linked to one or more second target gene(s) of the genetically engineered bacterium upon inversion by the SSR or integrase; and wherein:
(a) the first target gene is a glnA gene encoding a wild-type glutamine synthetase (GS) or variant thereof with improved catalytic activity in comparison to wild-type GS and the second target gene is a glnA gene encoding a wild-type GS with reduced levels of expression in comparison to the wild-type glnA gene or a GS variant with decreased catalytic activity in comparison to wild-type GS, and the fertilizer is ammonia, optionally wherein the wild-type GS comprises a protein sequence having at least 70%, 80%, 85%, 90%, 95%, 98%, or 99% identity to SEQ ID NO: 229, 230, 460-512, 519-521, or 530-533;
(b) the first target gene is a glnE gene encoding a wild-type GS adenylyltransferase protein and the second target gene is a glnE gene encoding a GS adenylyltransferase protein lacking an adenylyl removing domain which exhibits unidirectional adenylyltransferase (uAT) activity, and the fertilizer is ammonia, optionally wherein the protein encoded by the second target gene comprises a protein sequence having at least 70%, 80%, 85%, 90%, 95%, 98%, or 99% identity to SEQ ID NO: 16-24, or 25 or optionally wherein the second target gene comprises a DNA sequence having at least 70%, 80%, 85%, 90%, 95%, 98%, or 99% identity to SEQ ID NO: 1-14, or 15; or
(c) the first target gene is a nifL gene and the second target gene is a nifA gene, and the fertilizer is ammonia.

29. The method of embodiment 28, wherein the control element which is operably linked to said target genes comprises a constitutive promoter, an inducible promoter, or phosphate-sensitive promoter.

30. The method of embodiment 28 or 29, wherein the integrase is a serine integrase, optionally wherein the serine integrase is a phage PhiC31 serine integrase, IntS, IntM, IntG—ICEMcSym 1271, YdcL—ICEBs, or Int—ICE SXT/R39 integrase, and the SSRRS are attB and attP sites recognized respectively by the PhiC31, IntS, IntM, IntG, YdcL, or Sxt/R39 integrase.

31. The method of embodiment 28, wherein: (i) one or more SSR is a yeast flippase (FLP) recombinase and the SSRRS are FRT sites; or (ii) one or more SSR is a Cre-recombinase and the SSRRS are loxP sites.

32. The method of any one of embodiments 1-22, wherein the protein of interest is a first repressor protein and wherein the genetically engineered bacterium further comprises:
(i) a second control element comprising a promoter which is repressed by the first repressor protein and operably linked to a gene encoding a second repressor protein;
(ii) optionally a third control element comprising a promoter which is repressed by the second repressor protein and operably linked to a gene encoding a third repressor protein;
(iii) optionally a fourth control element comprising a promoter which is repressed by the third repressor protein and operably linked to a gene encoding a fourth repressor protein; and
(iv) a control element comprising a promoter which is repressed by the second, third, or fourth repressor protein and which is operably linked to a first target gene, optionally wherein the first, second, third, and/or fourth repressor protein(s) comprise(s) the lambda repressor (cI), the tet repressor (TetR), the lac repressor (LacI), a catalytically inactive RNA-guided DNA binding protein, a protein comprising a DNA-binding zinc finger domain, a transcription activator-like effector (TALE), or any variant thereof.

33. The method of embodiment 32, wherein the genetically engineered bacterium lacks (ii) and (iii), and the first target gene comprises: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s), and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase, and the agriculturally relevant compound is phosphate.

34. The method of embodiment 32, wherein the genetically engineered bacterium further comprises (ii) and (iii), and the first target gene comprises: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase, and the agriculturally relevant compound is phosphate.

35. The method of embodiment 32, wherein the genetically engineered bacterium further comprises (ii) and lacks (iii), and the first target gene is amtB, draT, glnA, glnB, glnK, glnZ, and/or nifL, and the agriculturally relevant compound is ammonia.

36. The method of embodiment 32, wherein first target gene encodes a transcriptional activator protein and wherein the genetically engineered bacterium further comprises a control element comprising a promoter which is activated by the transcriptional activator protein and operably linked to at least one second target gene.

37. The method of embodiment 32, wherein the genetically engineered bacterium comprises an even number of repressors and the first target gene comprises: (a) nfA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), a pyrroloquinoline (PQQ) synthase, and the agriculturally relevant compound is phosphate.

38. The method of embodiment 32, wherein the genetically engineered bacterium comprises an even number of repressors and the first target gene encodes an RNA sequence or protein comprising:
a. a GlnE protein lacking an adenylyl removing domain which exhibits unidirectional adenylyltransferase (uAT) activity and the fertilizer is ammonia;
b. a NifA protein, wherein the genetically engineered bacterium optionally comprises a loss-of-function mutation in any one or more of the amtB, draT, glnA, glnB, glnK, glnR, glnZ, or nifL genes, wherein the genetically engineered bacterium optionally comprises one or more heterologous genes from a wild-type or refactored nif or fix gene cluster and the fertilizer is ammonia;
c. a GlnR protein and the fertilizer is ammonia;
d. a glutaminase enzyme and the fertilizer is ammonia;
e. a protein product of a refactored nif or fix gene cluster and the fertilizer is ammonia;
f. a phytase enzyme and the agriculturally relevant compound is phosphate, optionally wherein the phytase enzyme comprises a cysteine phytase, a histidine acid phytase, or a beta-propeller phytase;
g. an acid phosphatase enzyme and the agriculturally relevant compound is phosphate, optionally wherein the acid phosphatase enzyme is encoded by an acpA, aphA, phoC, napA, napD, or napE gene or variant thereof;
h. a protein which stimulates organic acid release from the bacterium and the agriculturally relevant compound is phosphate, optionally wherein the protein which stimulates organic acid release comprises a gluconate dehydrogenase (GAD), a glucose dehydrogenase (GDH), or a pyrroloquinoline (PQQ) synthase;
i. a repressor protein, wherein the repressor protein binds a natural or synthetic DNA motif in the promoter of any one or more second target gene(s) of the genetically engineered bacterium and inhibits expression of any one or more of the protein product(s) of the second target gene(s) and/or wherein the repressor protein optionally comprises the lambda repressor (cI), the tet repressor (TetR), the lac repressor (LacI), a catalytically inactive RNA-guided DNA binding protein, a protein comprising a DNA-binding zinc finger domain, a transcription activator-like effector (TALE), or any variant thereof and/or optionally wherein the second target gene(s) is/are a glnA, amtB, glnB, glnK, glnZ, nifL, and/or draT gene and the fertilizer is ammonia;
j. a non-coding synthetic small RNA (sRNA); optionally wherein the non-coding synthetic small RNA (sRNA) binds a natural or synthetic DNA and/or RNA motif in the promoter, 5′ UTR, and/or coding region of any one or more second target gene(s) of the genetically engineered bacterium, optionally wherein the non-coding synthetic small RNA (sRNA) comprises a guide RNA that additionally binds an RNA-guided DNA endonuclease, an RNA-guided RNA endonuclease, or variant thereof and/or optionally wherein the second target gene(s) is/are a glnA, amtB, glnB, glnK, glnZ, nifL, and/or draT gene and the fertilizer is ammonia;
k. a site-specific recombinase (SSR) or integrase protein: wherein any one or more second target gene(s) of the genetically engineered bacterium and/or a promoter operatively linked thereto are flanked by site-specific recombinase recognition sites (SSRRS) in a direct configuration, optionally wherein the SSRRS comprise attL and attR sites and the genetically engineered bacterium comprises a gene encoding a recombinase directionality factor (RDF) and/or optionally wherein the second target gene(s) is/are a glnA, amtB, glnB, segment of glnE encoding an adenylyl-removing domain of a glutamine synthetase adenylyltransferase, glnK, glnZ, nifL, and/or draT gene and the fertilizer is ammonia;
l. a site-specific recombinase (SSR) or integrase protein: wherein any one or more second target gene(s), the promoter(s) thereof, and/or the 5′ UTR(s) thereof of the genetically engineered bacterium comprise(s) one or more internal synthetic SSRRS and wherein the gene(s), promoter(s), the 5′ UTR(s), and/or a segment(s) thereof is/are excised or inactivated after a recombination event; optionally wherein the genetically engineered bacterium further comprises a plasmid comprising an SSRRS; optionally wherein the genetically engineered bacterium further comprises one or more genes encoding a recombinase directionality factor (RDF); and/or optionally wherein the second target gene(s) is/are a glnA, amtB, glnB, glnE, glnK, glnZ, nifL, and/or draT gene and the fertilizer is ammonia;
m. a site-specific recombinase (SSR) or integrase protein, wherein any one or more second target gene(s), the promoter(s) thereof, and/or the 5′ UTR(s) thereof of the genetically engineered bacterium comprise(s) one or more internal integrative SSRRS and retain(s) activity of said second target gene(s), promoter(s), and/or 5′ UTR(s) and wherein the genetically engineered bacterium further comprises an integrative element comprising an SSRRS; optionally wherein the internal integrative SSRRS is/are an attB site(s), the integrative element comprises an SSRRS comprising an attP site; and/or optionally wherein the second target gene(s) is/are glnA, amtB, glnB, glnK, glnZ, nifL, and/or draT and the fertilizer is ammonia;
n. a site-specific recombinase (SSR) or integrase protein, wherein a control element comprising a promoter and/or at least a segment of a 5′ UTR: (i) is flanked by site-specific recombinase recognition sites (SSRRS) in an inverted configuration; and (ii) is operably linked to one or more second target gene(s) of the genetically engineered bacterium; optionally wherein the control element which is operably linked to said second target gene(s) comprises a constitutive promoter, an inducible promoter, or phosphate-sensitive promoter; and/or optionally wherein the second target gene(s) is/are glnA, amtB, glnB, glnK, glnZ, nifL, and/or draT and the fertilizer is ammonia;
o. a site-specific recombinase (SSR) or integrase protein, wherein a control element comprising a promoter and at least a segment of a 5′ UTR: (i) is flanked by site-specific recombinase recognition sites (SSRRS) in an inverted configuration; and (ii) is operably linked to one or more second target gene(s) of the genetically engineered bacterium upon inversion by the SSR or integrase; optionally wherein the control element which is operably linked to said second target gene(s) upon inversion by the SSR or integrase comprises a constitutive promoter, an inducible promoter, or phosphate-sensitive promoter; and/or optionally wherein the second target gene(s) is/are: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase, and the agriculturally relevant compound is phosphate;
p. a site-specific DNA endonuclease, wherein any one or more second target gene(s) of the genetically engineered bacterium comprise(s) one or more specific DNA sequence(s) recognized by the site-specific DNA endonuclease, optionally wherein the site-specific DNA endonuclease comprises an RNA-guided DNA endonuclease, a protein comprising a DNA-binding zinc finger domain, a transcription activator-like effector (TALE), a meganuclease, a homing endonuclease, or a restriction endonuclease, and/or optionally wherein the second target gene(s) is/are glnA, amtB, glnB, glnK, glnZ, nifL, and/or draT and the fertilizer is ammonia;
q. a protease which cleaves a protease specific recognition sequence (PSRS), wherein any one or more second target gene(s) of the genetically engineered bacterium comprise(s) one or more in-frame insertion(s) comprising DNA encoding the PSRS in the protein coding region of the second target gene(s), wherein the target protein product comprising the one or more in-frame insertion(s) has activity, and wherein cleavage of the target protein product(s) by the protease deactivates the target protein product(s), optionally wherein the location of the one or more in-frame insertion is given by Table 6, optionally wherein the second target gene(s) is/are under the control of a constitutive promoter, and/or optionally wherein the second target gene(s) is/are glnA, amtB, glnB, glnK, glnZ, nifL, and/or draT and the fertilizer is ammonia;
r. a first protein comprising a ClpAP ATP-dependent protease and a second protein comprising a ClpS Leu/N-recognin, wherein any one or more second target gene(s) of the genetically engineered bacterium encodes a third protein comprising an N-terminal-Leu, -Phe, -Trp, or -Tyr residue, and optionally wherein the first and second protein are operably linked to distinct control elements, and/or optionally wherein the second target gene(s) is/are glnA, amtB, glnB, glnK, glnZ, nifL, and/or draT and the fertilizer is ammonia;
s. a protease which cleaves a protease specific recognition sequence (PSRS), wherein any one or more second target gene(s) of the genetically engineered bacterium comprise(s) an in-frame insertion(s) of DNA encoding the PSRS at the N-terminus of the protein coding region of the gene(s) followed by a -Leu, -Phe, -Trp, or -Tyr residue and wherein cleavage of PSRS from the N-terminus of the protein(s) encoded by the gene(s) by the protease results in a protein comprising an N-terminal-Leu, -Phe, -Trp, or -Tyr residue which is degraded by native ClpS and ClpAP, optionally wherein the second target gene(s) is/are glnA, amtB, glnB, glnK, glnZ, nifL, and/or draT and the fertilizer is ammonia;
t. a transcriptional activator protein, wherein the transcriptional activator protein binds a natural or synthetic DNA motif in a promoter of a heterologous protease gene of the genetically engineered bacterium and activates expression of the protease, wherein any one or more second target gene(s) of the genetically engineered bacterium comprise(s) one or more in-frame insertion(s) of DNA encoding a PSRS, optionally wherein the transcriptional activator protein comprises a tet responsive element-binding tTA transcription factor or a transcriptional activator domain fused to a DNA targeting protein, optionally wherein the DNA targeting protein is a catalytically inactive RNA-guided DNA binding protein, a protein comprising a DNA-binding zinc finger domain, a transcription activator-like effector (TALE), or any variant thereof, optionally wherein the transcriptional activator domain is VP16, and/or optionally wherein the second target gene(s) is/are: glnA, amtB, glnB, glnK, glnZ, nifL, and/or draT and the fertilizer is ammonia;
u. a repressor protein, wherein the repressor protein binds a natural or synthetic DNA motif in a promoter of a heterologous protease gene of the genetically engineered bacterium and inhibits expression of the protease, wherein any one or more second target gene(s) of the genetically engineered bacterium comprise(s) an in-frame insertion of DNA encoding an N-terminal amino acid tag designed to render the protein product(s) of the second target gene(s) inactive, followed by a PSRS, optionally wherein the repressor protein comprises the lambda repressor (cI), the tet repressor (TetR), the lac repressor (LacI), a catalytically inactive RNA-guided DNA binding protein, a protein comprising a DNA-binding zinc finger domain, a transcription activator-like effector (TALE), or any variant thereof, and/or optionally wherein the second target gene(s) is/are glnA, amtB, glnB, glnK, glnZ, nifL, and/or draT and the fertilizer is ammonia;
v. a nanobody, wherein the nanobody binds a protein product of any one or more second target gene(s) of the genetically engineered bacterium, wherein binding of the nanobody to such protein product inhibits function of the protein product, optionally wherein the second target gene(s) is/are glnA, amtB, glnB, glnK, glnZ, nifL, and/or draT and the fertilizer is ammonia;
w. an aptamer, wherein the aptamer binds a protein product of any one or more second target gene(s) of the genetically engineered bacterium and inhibits function of any one or more of the protein product(s) of the second target gene(s), optionally wherein the second target gene(s) is/are glnA, amtB, glnB, glnK, glnZ, nifL, and/or draT and the fertilizer is ammonia; or
x. a transcriptional activator protein, wherein the transcriptional activator protein binds a natural or synthetic DNA motif in a promoter of any one or more second target gene(s) and increases expression of any one or more of the second target gene(s), optionally wherein the transcriptional activator protein comprises the tet responsive element-binding tTA transcription factor or a transcriptional activator domain fused to a DNA targeting protein, wherein the DNA targeting protein is optionally a catalytically inactive RNA-guided DNA binding protein, a protein comprising a DNA-binding zinc finger domain, a transcription activator-like effector (TALE), or any variant thereof, wherein the transcriptional activator domain is optionally VP16 and/or optionally wherein the second target gene(s) is/are any one or more of: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, and/or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase and the agriculturally relevant compound is phosphate.

39. The method of embodiment 32, wherein the genetically engineered bacterium comprises an odd number of repressors and the second target gene is amtB, draT, glnA, glnB, glnK, glnZ, and/or nifL, and the agriculturally relevant compound is ammonia.

40. The method of embodiment any one of embodiments 1-21, wherein the protein of interest is a first transcriptional activator protein and wherein the genetically engineered bacterium further comprises:
(i) a second control element comprising a promoter which is activated by the first transcriptional activator protein and operably linked to a gene encoding a second transcriptional activator protein;
(ii) optionally a third control element comprising a promoter which is activated by the second transcriptional activator protein and operably linked to a gene encoding a third transcriptional activator protein;
(iii) optionally a fourth control element comprising a promoter which is activated by the third transcriptional activator protein and operably linked to a gene encoding a fourth transcriptional activator protein; and
(iv) a control element comprising a promoter which is activated by the second, third, or fourth transcriptional activator protein and which is operably linked to a first target gene, optionally wherein the first, second, third, and/or fourth transcriptional activator protein comprise(s) the tet responsive element-binding tTA transcription factor or an activator domain fused to a DNA targeting protein, optionally wherein the DNA targeting protein is a catalytically inactive RNA-guided DNA binding protein, a protein comprising a DNA-binding zinc finger domain, a transcription activator-like effector (TALE), and/or any variant thereof, optionally wherein the activator domain is VP16.

41. The method of embodiment 40, wherein the first target gene comprises: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase, and the agriculturally relevant compound is phosphate.

42. The method of any one of embodiments 1-22, wherein the protein of interest is a repressor protein and wherein the genetically engineered bacterium further comprises:
(i) a second control element comprising a promoter which is repressed by the repressor protein and operably linked to a gene encoding a transcriptional activator protein; and
(ii) a third control element comprising a promoter which is activated by the transcriptional activator protein and operably linked to a target gene.

43. The method of embodiment 42, wherein the target gene is a glnA, amtB, glnB, glnK, glnZ, nifL, and/or draT gene and the agriculturally relevant compound is ammonia.

44. The method of any one of embodiments 1-22, wherein the protein of interest is a transcriptional activator protein and wherein the genetically engineered bacterium further comprises:
(i) a second control element comprising a promoter which is activated by the transcriptional activator protein and operably linked to a gene encoding a repressor protein; and
(ii) a third control element comprising a promoter which is repressed by the repressor and operably linked to a target gene.

45. The method of embodiment 44, wherein the target gene is a glnA, amtB, glnB, glnK, glnZ, nifL, and/or draT gene and the agriculturally relevant compound is ammonia.

46. The method of embodiment 22, wherein the glnA, amtB, glnB, glnK, glnZ, nifL, and/or draT gene in (n), (o), (p), or (q) is under the control of a heterologous constitutive promoter.

47. The method of embodiment 38, wherein the glnA, amtB, glnB, glnK, glnZ, nifL, and/or draT gene in (q), (r), (s), or (t) is under the control of a heterologous constitutive promoter.

48. The method of any one of embodiments 1-21, wherein the plant nutrient is phosphate and the protein of interest comprises:
a. a phytase enzyme, optionally wherein the phytase enzyme comprises a cysteine phytase, a histidine acid phytase, or a beta-propeller phytase, optionally wherein the phytase enzyme comprises a protein sequence having at least 70%, 75%, 80%, 85%, 90%, 95%, 98%, or 99% sequence identity to SEQ ID NO: 58-138, or 139;
b. an acid phosphatase enzyme, optionally wherein the acid phosphatase is encoded by an acpA, aphA, phoC, napA, napD, or napE gene or variant thereof;
c. a protein which stimulates organic acid release from the bacterium, optionally wherein the protein comprises a gluconate dehydrogenase (GAD), a glucose dehydrogenase (GDH), a pyrroloquinoline (PQQ) synthase including pqqFABCDEG, or any combination of GAD, GDH, and PQQ; or,
d. any combination of proteins of a, b, or c.

49. The method of any one of embodiments 1 to 48, wherein the heterologous gene expression cassette is introduced into bacteria originally isolated from a plant growth medium or a plant to obtain the genetically engineered bacteria.

50. The method of any one of embodiments 1 to 49, wherein the bacterium is selected from the group of gram-negative bacteria.

51. The method of any one of embodiments 1 to 49, wherein the bacteria are selected from the groups of bacteria of the taxonomic classes of alphaproteobacteria, betaproteobacteria, and gammaproteobacteria.

52. The method of any one of embodiments 1 to 49, wherein the bacteria are selected from the groups of bacteria in the taxonomic genera of Acetobacter, Acidothermus, Acinetobacter, Agrobacterium, Aromatoleum, Arthrobacter, Azoarcus, Azorhizobium, Azospirillum, Azotobacter, Bacillus, Bifidobacterium, Bradyrhizobium, Burkholderia, Conexibacter, Curtobacterium, Ensifer, Enterobacter, Erwinia, Escherichia, Flavobacterium, Frankia, Gaiella, Gluconacetobacter, Gluconobacter, Herbaspirillum, Klebsiella, Kosakonia, Lactobacillus, Lactococcus, Lysinibacillus, Maritimibacter, Methylobacterium, Nitrosocosmicus, Nitrososphaera, Paenarthrobacter, Paenibacillus, Panotea, Pediococcus, Peribacillus, Phytobacter, Priestia, Pseudarthrobacter, Pseudomonas, Rahnella, Rhizobium, Rhodococcus, Rhodoplanes, Rhodopseudomonas, Rhodospirillum, Serratia, Solirubrobacter, Sphingobacterium, Sphingomonas, Stenotrophomonas, Streptomyces, Stutzerimonas, Variovorax, Xanthobacter, and Yoonia, optionally wherein the bacteria are selected from at least one of the taxonomic genera selected from the group consisting of Herbaspirillum, Azospirillum, Kosakonia, Pseudomonas, Enterobacter, Klebsiella, Paenibacillus, Phytobacter, Rahnella, Sphingomonas, or Variovorax.

53. The method of any one of embodiments 1 to 52, wherein the phosphate concentration in the plant growth medium is reduced as a consequence of:
i. growth of resident soil organisms, microorganisms, and/or the genetically engineered bacterium in the plant growth medium, and/or
ii. growth of a plant in the plant growth medium.

54. The method of any one of embodiments 1 to 53, wherein the plant growth medium has been treated by adding inorganic phosphate to the plant growth medium at a rate of about 3 g/m² to about 15 g/m².

55. The method of any one of embodiments 1 to 54, wherein the threshold concentration of phosphate in the plant growth medium which activates expression of the RNA or protein of interest is about 0 μM to any one of about 1 μM, 2 μM, 5 μM, 7 μM, 10 μM, 20 μM, 30 μM, 40 μM, or 50 μM.

56. The method of any one of embodiments 1 to 55, wherein the genetically engineered bacterium is placed into the plant growth medium and/or in contact with the plant:
(i) by foliar application to the plant;
(ii) by an in furrow application, fumigation, and/or soil drench;
(iii) with a seed in form of a seed treatment wherein the seed is at least partially coated with a composition comprising the genetically engineered bacterium;
(iv) with a seed in the form of bio-priming where the seed is imbibed with an aqueous composition comprising the genetically engineered bacterium before planting; and/or
(v) with a root dip transplant whereby a seedling root system is dipped in an aqueous composition comprising the genetically engineered bacterium.

57. The method of any one of embodiments 1 to 56, further comprising;
a. determining leaf nitrogen and/or chlorophyll concentrations in a plant grown in the plant growth medium; and
b. placing or re-applying the genetically engineered bacterium into the plant growth medium and/or in contact with the plant when the leaf nitrogen and/or chlorophyll concentrations in the plant are sub-optimal for yield.

58. A genetically engineered bacterium comprising a heterologous expression cassette comprising at least one nucleic acid sequence coding for at least one RNA sequence or protein of interest operably linked to a to a control element comprising a phosphate-sensitive promoter, wherein said at least one RNA sequence or protein of interest is or causes the production of at least one agriculturally relevant compound.

59. The genetically engineered bacterium of embodiment 58, wherein the control element and/or the heterologous gene expression cassette further comprises at least a segment of a 5′ untranslated region (5′ UTR) which is operably linked to the phosphate-sensitive promoter and/or the heterologous gene expression cassette, optionally wherein the 5′ UTR comprises a 5′ UTR sequence which has at least 85% sequence identity to an endogenous 5′ UTR sequence which is operably linked to an endogenous phosphate-sensitive promoter.

60. The genetically engineered bacterium of embodiment 59, wherein the phosphate-sensitive promoter and/or the segment of the 5′ UTR which is operably linked to the phosphate-sensitive promoter comprises at least one copy of an operably linked Pho box, wherein the Pho box comprises the sequence of SEQ ID NO: 234-397, 525-528, or 529, and sequences with at least 95% identity to any one of SEQ ID NO: 234-397, 525-528, or 529, optionally wherein the Pho box comprises SEQ ID NO: 376, 377, 383-396, 397, 525-528, or 529.

61. The genetically engineered bacterium of embodiment 59 or 60, wherein the phosphate-sensitive promoter and/or segment the of the 5′ UTR is a promoter and/or 5′ UTR derived from at least one gram-negative bacterium.

62. The genetically engineered bacterium of embodiment 59 or 60, wherein the phosphate-sensitive promoter and/or the segment of the 5′ UTR is a promoter and/or 5′ UTR derived from at least one bacterium of the taxonomic classes of alphaproteobacteria, betaproteobacteria, and gammaproteobacteria.

63. The genetically engineered bacterium of embodiment 59 or 60, wherein the phosphate-sensitive promoter and/or the segment of the 5′ UTR is a promoter and/or segment of a 5′ UTR derived from at least one bacterium of a genus selected from the group consisting of Acetobacter, Acidothermus, Acinetobacter, Agrobacterium, Aromatoleum, Arthrobacter, Azoarcus, Azorhizobium, Azospirillum, Azotobacter, Bacillus, Bifidobacterium, Bradyrhizobium, Burkholderia, Conexibacter, Curtobacterium, Ensifer, Enterobacter, Erwinia, Escherichia, Flavobacterium, Frankia, Gaiella, Gluconacetobacter, Gluconobacter, Herbaspirillum, Klebsiella, Kosakonia, Lactobacillus, Lactococcus, Lysinibacillus, Maritimibacter, Methylobacterium, Nitrosocosmicus, Nitrososphaera, Paenarthrobacter, Paenibacillus, Panotea, Pediococcus, Peribacillus, Phytobacter, Priestia, Pseudarthrobacter, Pseudomonas, Rahnella, Rhizobium, Rhodococcus, Rhodoplanes, Rhodopseudomonas, Rhodospirillum, Serratia, Solirubrobacter, Sphingobacterium, Sphingomonas, Stenotrophomonas, Streptomyces, Stutzerimonas, Variovorax, Xanthobacter, and Yoonia, optionally wherein the phosphate-sensitive promoter and/or the segment of the 5′ UTR is derived from at least one bacterium of a genus selected from the group consisting of Azospirillum, Herbaspirillum, Pseudomonas, Kosakonia, Enterobacter, Klebsiella, Paenibacillus, Phytobacter, Pseudomonas, Rahnella, Sphingomonas, and Variovorax.

64. The genetically engineered bacterium of any one of embodiments 59-63, wherein the phosphate-sensitive promoter and/or the segment of the 5′ UTR comprises a promoter and/or a segment of a 5′ UTR of a phoA, phoX, phy, pstS gene, a variant thereof, or a combination thereof.

65. The genetically engineered bacterium of any one of embodiments 59-63, wherein the phosphate-sensitive promoter and/or the segment of the 5′ UTR comprises a promoter and/or a segment of a 5′ UTR of a promoter comprising the DNA sequence of SEQ ID NO: 417-428, 515, 516, or 518, a phosphate-sensitive promoter element of a promoter comprising the DNA sequence of SEQ ID NO: 417-428, 515, 516, 518, a variant thereof, or a combination thereof, optionally wherein the variant has at least 76%, 80%, 85%, 90%, 95%, 98%, or 99% sequence identity to SEQ ID NO: 417-428, 515, 516, or 518.

66. The genetically engineered bacterium of any one of embodiments 59-65, wherein the phosphate-sensitive promoter and/or the segment of the 5′ UTR comprises a promoter of a gene encoding:
i. a PhoA protein comprising a protein sequence selected from the group consisting of SEQ ID NO: 398, 400, 402, 404, and 406 or having an identity of at least 80%, 85%, 90%, 95%, 98%, or 99% with any one of SEQ ID NO: 398, 400, 402, 404, or 406;
ii. a PhoX protein comprising a protein sequence selected from the group consisting of SEQ ID NO: 408, 410, 411, 412, and 413 or having an identity of at least 80%, 85%, 90%, 95%, 98%, or 99% with any one of SEQ ID NO: 408, 410, 411, 412, and 413;
iii. a Phy protein comprising a protein sequence selected from the group consisting of SEQ ID NO: 414, 57-138, and 139 or having an identity of at least 80%, 85%, 90%, 95%, 98%, or 99% with any one of SEQ ID NO: 414, 57-138, or 139; and/or
iv. a PstS protein comprising a protein sequence selected from the group consisting of SEQ ID NO: 399, 401, 403, 405, 407, 409, 415, 416, 513, 523, and 524 or having an identity of at least 76%, 80%, 85%, 90%, 95%, 98%, or 99% with any one of SEQ ID NO: 399, 401, 403, 405, 407, 409, 415, 416, 513, 523, or 524.

67. The genetically engineered bacterium of any one of embodiments 59-66, wherein the promoter region and/or the segment of the 5′ UTR comprises at least 50 nucleotides of DNA located upstream of a start codon of a gene encoding a PhoA, PhoX, Phy, or PstS protein, optionally wherein the promoter region and/or the segment of the 5′ UTR comprises about 50 to about 250 nucleotides of DNA located upstream of said start codon of said gene encoding said PhoA, PhoX, Phy, or PstS protein.

68. The genetically engineered bacterium of any one of embodiments 59-67, wherein the phosphate-sensitive promoter and/or the segment of the 5′ UTR comprises:
i. a phoA promoter comprising the DNA sequence of SEQ ID NO: 419;
ii. a phoX promoter comprising the DNA sequence of SEQ ID NO: 426;
iii. a phy promoter comprising the DNA sequence of SEQ ID NO: 427;
iv. a pstS promoter comprising a DNA sequence selected from the group consisting of SEQ ID NO: 417, 418, 420, 421, 422, 423, 424, 425, 428, 515, 517, and 518; and/or
v. a Pliar53 promoter comprising the DNA sequence of SEQ ID NO: 459;
vi. a variant of the promoters under i. to v. having an identity of at least 76%, 80%, 90%, 95%, 98%, or 99% with any of SEQ ID NO: 417, 418-428, 459, 515, 517, or 518 515, 517, or 518, wherein said variant comprises at least one Pho box and/or retains at least one Pho box present in each promoter and wherein said variant promoters are activated by a decrease in phosphate concentration to about 1 μM to about 1 μM, 2 μM, 5 μM, 7 μM, 10 μM, 20 μM, 30 μM, 40 μM, or 50 μM.

69. The genetically engineered bacterium of any one of embodiments 59-68, wherein the phosphate-sensitive promoter and/or the segment of the 5′ UTR or its variant is derived from the same genus or species as the genetically engineered bacterium.

70. The genetically engineered bacterium of any one of embodiments 58-69, wherein the heterologous gene expression cassette is integrated at a location in the chromosome of the genetically engineered bacterium which does not comprise the location of an endogenous phosphate-sensitive promoter.

71. The genetically engineered bacterium of any one of embodiments 58-70, wherein the expression cassette further comprises one or more elements comprising:
a. a ribosome binding site (RBS), wherein the RBS is operably linked to the nucleic acid sequence coding for the protein of interest and optionally wherein the RBS is an RBS having at least 95% sequence identity to SEQ ID NO: 429-454, or 455; and/or
b. a terminator sequence (TS), wherein the TS is operably linked to the nucleic acid sequence coding for the RNA sequence or protein of interest and optionally wherein the TS is a TS having at least 95% sequence identity to SEQ ID NO: 456, 457, or 458.

72. The genetically engineered bacterium of any one of embodiments 58 to 71, wherein the at least one agriculturally relevant compound is selected from the group consisting of:
a. at least one fertilizer or plant nutrient, optionally wherein the fertilizer or plant nutrient is selected from the group consisting of ammonia, ammonium, bioavailable carbon, calcium, iron, nitrate, nitrite, nitrogen, potassium, phosphate, sulfur, urea, zinc, a combination thereof, and a mixture thereof;
b. at least one pesticide, optionally wherein the pesticide is an RNA- and/or protein-based fungicide, insecticide, nematicide, antibacterial agent, and/or antiviral agent;
c. at least one phytohormone or plant growth regulator, optionally wherein the phytohormone or plant growth regulator is an auxin, a cytokinin, a gibberellin, abscisic acid, a brassinosteroid, jasmonic acid, a polyamine, a strigolactone, trehalose, and/or a volatile organic compound, or optionally wherein the phytohormone or plant growth regulator is cytokinin, indole butyric acid, and/or combinations thereof; and
d. at least one carbon-containing compound, optionally wherein the carbon-containing compound is bicarbonate, carbonate, CaCO₃, MgCO₃, CaMg(CO₃)₂, polyhydroxybutyrate, melanin, chitin, and/or combinations thereof.

73. The genetically engineered bacterium of embodiment 72, wherein the RNA sequence or protein of interest encoded by the heterologous gene expression cassette and operably linked to the control element comprises:
(a) a GlnE protein lacking an adenylyl removing domain which exhibits unidirectional adenylyltransferase (uAT) activity and the fertilizer is ammonia, optionally wherein the GlnE protein lacking an adenylyl removing domain comprises a protein sequence having at least 70%, 80%, 85%, 90%, 95%, 98%, or 99% identity to SEQ ID NO: 16-24, or 25 or optionally wherein the gene encoding the GlnE protein lacking an adenylyl removing domain comprises a DNA sequence having at least 70%, 80%, 85%, 90%, 95%, 98%, or 99% identity to SEQ ID NO: 1-14, or 15;
(b) a NifA protein, wherein the genetically engineered bacterium optionally comprises a loss-of-function mutation in any one or more of the amtB, draT, glnA, glnB, glnK, glnR, glnZ, or nifL genes, wherein the genetically engineered bacterium optionally comprises one or more heterologous genes from a wild-type or refactored nif or fix gene cluster and the fertilizer is ammonia;
(c) a GlnR protein and the fertilizer is ammonia;
(d) a glutaminase enzyme and the fertilizer is ammonia;
(e) a protein product of a refactored nif or fix gene cluster and the fertilizer is ammonia;
(f) a repressor protein, wherein the repressor protein binds a natural or synthetic DNA motif in the promoter of any one or more first target gene(s) of the genetically engineered bacterium and inhibits expression of any one or more of the protein products of the first target genes and/or wherein the repressor protein optionally comprises the lambda repressor (cI), the tet repressor (TetR), the lac repressor (LacI), a catalytically inactive RNA-guided DNA binding protein, a protein comprising a DNA-binding zinc finger domain, a transcription activator-like effector (TALE), or any variant thereof and/or optionally wherein the first target gene(s) is/are a glnA, amtB, glnB, glnK, glnZ, nifL, and/or draT gene and the fertilizer is ammonia;
(g) a non-coding synthetic small RNA (sRNA); optionally wherein the non-coding synthetic small RNA (sRNA) binds a natural or synthetic DNA and/or RNA motif in the promoter, 5′ UTR, and/or coding region of any one or more first target gene(s) of the genetically engineered bacterium, optionally wherein the non-coding synthetic small RNA (sRNA) comprises a guide RNA that additionally binds an RNA-guided DNA endonuclease, RNA-guided RNA endonuclease, or variant thereof and/or optionally wherein the first target gene(s) is/are a glnA, amtB, glnB, glnK, glnZ, nifL, and/or draT gene and the fertilizer is ammonia;
(h) a site-specific recombinase (SSR) or integrase protein: wherein any one or more first target gene(s) of the genetically engineered bacterium and/or a promoter operatively linked thereto are flanked by site-specific recombinase recognition sites (SSRRS) in a direct configuration, optionally wherein the SSRRS comprise attL and attR sites and the genetically engineered bacterium comprises a gene encoding a recombinase directionality factor (RDF) and/or optionally wherein the first target gene(s) is/are a glnA, amtB, glnB, segment of glnE encoding an adenylyl-removing domain of a glutamine synthetase adenylyltransferase, glnK, glnZ, nifL, and/or draT gene and the fertilizer is ammonia;
(i) a site-specific recombinase (SSR) or integrase protein: wherein any one or more first target gene(s), the promoter(s) thereof, and/or the 5′ UTR(s) thereof of the genetically engineered bacterium comprise(s) one or more internal synthetic SSRRS and wherein the gene(s), promoter(s), the 5′ UTR(s), and/or a segment(s) thereof is/are excised or inactivated after a recombination event; optionally wherein the genetically engineered bacterium further comprises a plasmid comprising an SSRRS; optionally wherein the genetically engineered bacterium further comprises one or more genes encoding a recombinase directionality factor (RDF); and/or optionally wherein the first target gene(s) is/are a glnA, amtB, glnB, glnE, glnK, glnZ, nifL, and/or draT gene and the fertilizer is ammonia;
(j) a site-specific recombinase (SSR) or integrase protein, wherein any one or more first target gene(s), the promoter(s) thereof, and/or the 5′ UTR(s) thereof of the genetically engineered bacterium comprise(s) one or more internal integrative SSRRS and retain(s) activity of said first target gene(s), promoter(s), and/or 5′ UTR(s) and wherein the genetically engineered bacterium further comprises an integrative element comprising an SSRRS; optionally wherein the internal integrative SSRRS is/are an attB site(s) and the integrative element comprises an SSRRS comprising an attP site; and/or optionally wherein the target gene(s) is/are glnA, amtB, glnB, glnK, glnZ, nifL, and/or draT and the fertilizer is ammonia;
(k) a site-specific recombinase (SSR) or integrase protein, wherein a control element comprising a promoter and/or at least a segment of a 5′ UTR: (i) is flanked by site-specific recombinase recognition sites (SSRRS) in an inverted configuration; and (ii) is operably linked to one or more first target gene(s) of the genetically engineered bacterium; optionally wherein the control element which is operably linked to said first target gene(s) comprises a constitutive promoter, an inducible promoter, or phosphate-sensitive promoter; and/or optionally wherein the first target gene(s) is/are glnA, amtB, glnB, glnK, glnZ, nifL, and/or draT and the fertilizer is ammonia;
(l) a site-specific recombinase (SSR) or integrase protein, wherein a control element comprising a promoter and at least a segment of a 5′ UTR: (i) is flanked by site-specific recombinase recognition sites (SSRRS) in an inverted configuration; and (ii) is operably linked to one or more first target gene(s) of the genetically engineered bacterium upon inversion by the SSR or integrase; optionally wherein the control element which is operably linked to said first target gene(s) upon inversion by the SSR or integrase comprises a constitutive promoter, an inducible promoter, or a phosphate-sensitive promoter; and/or optionally wherein the first target gene(s) is/are: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase, and the agriculturally relevant compound is phosphate;
(m) a site-specific DNA endonuclease, wherein any one or more first target gene(s) of the genetically engineered bacterium comprise(s) one or more specific DNA sequence(s) recognized by the site-specific DNA endonuclease, optionally wherein the site-specific DNA endonuclease comprises an RNA-guided DNA endonuclease, a protein comprising a DNA-binding zinc finger domain, a transcription activator-like effector (TALE), a meganuclease, a homing endonuclease, or a restriction endonuclease, and/or optionally wherein the first target gene(s) is/are glnA, amtB, glnB, glnK, glnZ, nifL, and/or draT and the fertilizer is ammonia;
(n) a protease which cleaves a protease specific recognition sequence (PSRS), wherein any one or more first target gene(s) of the genetically engineered bacterium comprise(s) one or more in-frame insertion(s) comprising DNA encoding the PSRS in the protein coding region of the first target gene(s), wherein the target protein product comprising the one or more in-frame insertion(s) has activity, and wherein cleavage of the target protein product(s) by the protease deactivates the target protein product(s), optionally wherein the location of the one or more in-frame insertion(s) is given by Table 6, optionally wherein the first target gene(s) is/are under the control of a constitutive promoter, and/or optionally wherein the first target gene(s) is/are glnA, amtB, glnB, glnK, glnZ, nifL, and/or draT and the fertilizer is ammonia;
(o) a first protein of interest comprising a ClpAP ATP-dependent protease and a second protein of interest comprising a ClpS Leu/N-recognin, wherein any one or more first target gene(s) of the genetically engineered bacterium encodes a protein comprising an N-terminal-Leu, -Phe, -Trp, or -Tyr residue, and optionally wherein the first and second protein of interest are operably linked to distinct control elements, and/or optionally wherein the first target gene(s) is/are glnA, amtB, glnB, glnK, glnZ, nifL, and/or draT and the fertilizer is ammonia;
(p) a protease which cleaves a protease specific recognition sequence (PSRS), wherein any one or more first target gene(s) of the genetically engineered bacterium comprise(s) an in-frame insertion(s) of DNA encoding the PSRS at the N-terminus of the protein coding region of the gene followed by a -Leu, -Phe, -Trp, or -Tyr residue and wherein cleavage of the PSRS from the N-terminus of the protein(s) encoded by the gene(s) by the protease results in a protein comprising an N-terminal-Leu, -Phe, -Trp, or -Tyr residue which is degraded by native ClpS and ClpAP, optionally wherein the first target gene(s) is/are glnA, amtB, glnB, glnK, glnZ, nifL, and/or draT and the fertilizer is ammonia;
(q) a transcriptional activator protein, wherein the transcriptional activator protein binds a natural or synthetic DNA motif in a promoter of a heterologous protease gene of the genetically engineered bacterium and activates expression of the protease, wherein any one or more first target gene(s) of the genetically engineered bacterium comprise(s) one or more in-frame insertion(s) of DNA encoding a PSRS, optionally wherein the transcriptional activator protein comprises a tet responsive element-binding tTA transcription factor or a transcriptional activator domain fused to a DNA targeting protein, optionally wherein the DNA targeting protein is a catalytically inactive RNA-guided DNA binding protein, a protein comprising a DNA-binding zinc finger domain, a transcription activator-like effector (TALE), or any variant thereof, optionally wherein the transcriptional activator domain is VP16, and/or optionally wherein the first target gene(s) is/are: glnA, amtB, glnB, glnK, glnZ, nifL, and/or draT and the fertilizer is ammonia;
(r) a repressor protein, wherein the repressor protein binds a natural or synthetic DNA motif in a promoter of a heterologous protease gene of the genetically engineered bacterium and inhibits expression of the protease, wherein any one or more first target gene(s) of the genetically engineered bacterium comprise(s) an in-frame insertion of DNA encoding an N-terminal amino acid tag designed to render the protein product(s) of the first target gene(s) inactive, followed by a PSRS, optionally wherein the repressor protein comprises the lambda repressor (cI), the tet repressor (TetR), the lac repressor (LacI), a catalytically inactive RNA-guided DNA binding protein, a protein comprising a DNA-binding zinc finger domain, a transcription activator-like effector (TALE), or any variant thereof, and/or optionally wherein the first target gene(s) is/are glnA, amtB, glnB, glnK, glnZ, nifL, and/or draT and the fertilizer is ammonia;
(s) a nanobody, wherein the nanobody binds a protein product of any one or more first target gene(s) of the genetically engineered bacterium, wherein binding of the nanobody to such protein product inhibits function of the protein product, optionally wherein the first target gene(s) is/are glnA, amtB, glnB, glnK, glnZ, nifL, and/or draT and the fertilizer is ammonia;
(t) an aptamer, wherein the aptamer binds a protein product of any one or more first target gene(s) of the genetically engineered bacterium and inhibits function of any one or more of the protein product(s) of the first target gene(s), optionally wherein the first target gene(s) is/are glnA, amtB, glnB, glnK, glnZ, nifL, and/or draT and the fertilizer is ammonia; or
(u) a transcriptional activator protein, wherein the transcriptional activator protein binds a natural or synthetic DNA motif in a promoter of any one or more first target gene(s) and increases expression of any one or more of the first target gene(s), optionally wherein the transcriptional activator protein comprises the tet responsive element-binding tTA transcription factor or a transcriptional activator domain fused to a DNA targeting protein, optionally wherein the DNA targeting protein is a catalytically inactive RNA-guided DNA binding protein, a protein comprising a DNA-binding zinc finger domain, a transcription activator-like effector (TALE), or any variant thereof, optionally wherein the transcriptional activator domain is VP16 and/or optionally wherein the first target gene(s) is/are any one or more of: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, and/or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase and the agriculturally relevant compound is phosphate.

74. The genetically engineered bacterium of embodiment 73, wherein the integrase is a serine integrase, optionally wherein the serine integrase is a phage PhiC31 serine integrase, IntS, IntM, IntG—ICEMcSym 1271, YdcL—ICEBs, or Int—ICE SXT/R39 integrase, and the SSRRS are attB and attP sites recognized respectively by the PhiC31, IntS, IntM, IntG, YdcL, or Sxt/R39 integrase.

75. The genetically engineered bacterium of embodiment 73, wherein: (i) one or more SSR is a yeast flippase (FLP) recombinase and the SSRRS are FRT sites; or (ii) one or more SSR is a Cre-recombinase and the SSRRS are loxP sites.

76. The genetically engineered bacterium of embodiment 73, wherein a PSRS is inserted between any one or more of the pairs of amino acid residues corresponding to:
(i) amino acid residues 98 and 99, 121 and 122, 279 and 280, and/or 285 and 286 of the glutamine synthetase (GS) polypeptide of SEQ ID NO: 229;
(ii) amino acid residues 98 and 99, 119 and 120, 283 and 284, and/or 298 and 299 of the glutamine synthetase (GS) polypeptide of SEQ ID NO: 230;
(iii) amino acid residues 448 and 449, 480 and 481, 505 and 506, 528 and 529, and/or 624 and 625 of the GlnE polypeptide of SEQ ID NO: 231;
(iv) amino acid residues 527 and 528, 537 and 538, and/or 547 and 548 of the GlnE polypeptide of SEQ ID NO: 232; or
(v) amino acid residues 422 and 423 and/or 608 and 609 of the GlnE polypeptide of SEQ ID NO: 233.

77. The genetically engineered bacterium of embodiment 73 or 76, wherein the protease or heterologous protease:
a. comprises a tobacco etch virus (TEV) protease and the PSRS comprises the peptide EXXYXQ-(S/G) or ENLYFQ-(S/G/A/M/C/H), wherein X is any amino acid and the TEV protease cleaves between the Q and S, G, A, M, C, or H residues;
b. comprises a tobacco vein mottling virus (TVMV) protease and the PSRS comprises the peptide ETVRFQ-(G/S), wherein the TVMV protease cleaves between the Q and S or G residues;
c. comprises a sunflower mild mosaic virus (SMMV) protease and the PSRS comprises the peptide EEIHLQ-(S/G), wherein the SMMV protease cleaves between the Q and S or G residues;
d. comprises a turnip mosaic virus (TrMV) protease and the PSRS comprises the peptide VXHQ or VRHQ-S, wherein X is any amino acid and the TrMV protease cleaves C-terminal to the Q residue;
e. comprises a soybean mosaic virus (SMV) protease and the PSRS comprises the peptide XVXXQ-(G/S), wherein X is any amino acid and the SMV protease cleaves between Q and S or G residues;
f. comprises a plum pox virus (PPV) protease and the PSRS comprises the peptide NVVVHQ-A, wherein the PPV protease cleaves between the Q and the A residue;
g. comprises a hepatitis C virus (HCV) NS3 protease and the PSRS comprises the peptide (D/E)XXXXC(A/S), wherein X is any amino acid and the HCV protease cleaves between the C and the A or S residues;
h. comprises an enterokinase and the PSRS comprises the peptide DDDDK, wherein the enterokinase cleaves C-terminal to the K residue;
i. comprises a Factor Xa protease and the PSRS comprises the peptide I(D/E)GR, wherein the Factor Xa protease cleaves C-terminal to the R residue; or
j. comprises a furin protease and the PSRS comprises the peptide RX(K/R)R, wherein the furin protease cleaves C-terminal to the C-terminal R residue.

78. The genetically engineered bacterium of embodiment 72, wherein:
(i) the plant nutrient is zinc or potassium and the protein of interest is a gluconate dehydrogenase (GAD) enzyme;
(ii) the plant nutrient is iron and the protein of interest is a siderophore biosynthetic and transport proteins optionally selected from a dhbACDEBF gene cluster; a non-ribosomal peptide synthetase (NRPS), polyketide synthase (PKS), and NRPS-independent siderophore synthetase (NIS); and major facilitator superfamily (MFS) transporters (ymfE), TonB, ExbD, and/or ExbB;
(iii) the phytohormone is auxin and the protein is an IpdC or IaaM protein;
(iv) the plant nutrient is ammonia and α-ketobutyrate and the protein is ACC deaminase;
(v) the plant growth regulator is a volatile organic compound and the proteins are Glyceraldehyde-3-Phosphate Dehydrogenase (GAPDH) and 2,3-butanediol Dehydrogenase (BDH);
(vi) the agriculturally relevant compound is trehalose and the protein(s) of interest is OtsA, OtsB, and TreS or TreS; or
(vii) the carbon containing compound is calcium carbonate and the protein of interest is a beta-carbonic anhydrase or alpha-carbonic anhydrase.

79. The genetically engineered bacterium of embodiment 72 or 73, wherein the protein of interest comprises a site-specific recombinase (SSR) or integrase protein, wherein a control element comprising a promoter and/or at least a segment of a 5′ UTR: (i) is flanked by site-specific recombinase recognition sites (SSRRS) in an inverted configuration; (ii) is operably linked to one or more first target gene(s) of the genetically engineered bacterium; and (iii) is operably linked to one or more second target gene(s) of the genetically engineered bacterium upon inversion by the SSR or integrase; and wherein:
(a) the first target gene is a glnA gene encoding a wild-type glutamine synthetase (GS) or variant thereof with improved catalytic activity in comparison to wild-type GS and the second target gene is a glnA gene encoding a wild-type GS with reduced levels of expression in comparison to the wild-type glnA gene or a GS variant with decreased catalytic activity in comparison to wild-type GS, and the fertilizer is ammonia, optionally wherein the wild-type GS comprises a protein sequence having at least 70%, 80%, 85%, 90%, 95%, 98%, or 99% identity to SEQ ID NO: 229, 230, 460-512, 519-521, 530-532, or 533;
(b) the first target gene is a glnE gene encoding a wild-type GS adenylyltransferase protein and the second target gene is a glnE gene encoding a GS adenylyltransferase protein lacking an adenylyl removing domain which exhibits unidirectional adenylyltransferase (uAT) activity, and the fertilizer is ammonia, optionally wherein the protein encoded by the second target gene comprises a protein sequence having at least 70%, 80%, 85%, 90%, 95%, 98%, or 99% identity to SEQ ID NO: 16-24, or 25 or optionally wherein the second target gene comprises a DNA sequence having at least 70%, 80%, 85%, 90%, 95%, 98%, or 99% identity to SEQ ID NO: 1-14, or 15; or
(c) the first target gene is a nifL gene and the second target gene is a nifA gene, and the fertilizer is ammonia.

80. The genetically engineered bacterium of embodiment 79, wherein the control element which is operably linked to said target genes comprises a constitutive promoter, an inducible promoter, or a phosphate-sensitive promoter.

81. The genetically engineered bacterium of embodiment 79 or 80, wherein the integrase is a serine integrase, optionally wherein the serine integrase is a phage PhiC31 serine integrase, IntS, IntM, IntG—ICEMcSym 1271, Yd-L—ICEBs, or I-t—ICE SXT/R39 integrase, and the SSRRS are attB and attP sites recognized respectively by the PhiC31, IntS, IntM, IntG, YdcL, or Sxt/R39 integrase.

82. The genetically engineered bacterium of embodiment 79 or 80, wherein: (i) one or more SSR is a yeast flippase (FLP) recombinase and the SSRRS are FRT sites; or (ii) one or more SSR is a Cre-recombinase and the SSRRS are loxP sites.

83. The genetically engineered bacterium of any one of embodiments 1-73, wherein the protein of interest is a first repressor protein and wherein the genetically engineered bacterium further comprises:
(i) a second control element comprising a promoter which is repressed by the first repressor protein and operably linked to a gene encoding a second repressor protein;
(ii) optionally a third control element comprising a promoter which is repressed by the second repressor protein and operably linked to a gene encoding a third repressor protein;
(iii) optionally a fourth control element comprising a promoter which is repressed by the third repressor protein and operably linked to a gene encoding a fourth repressor protein; and
(iv) a control element comprising a promoter which is repressed by the second, third, or fourth repressor protein and which is operably linked to a first target gene, optionally wherein the first, second, third, and/or fourth repressor protein(s) optionally comprise(s) the lambda repressor (cI), the tet repressor (TetR), the lac repressor (LacI), a catalytically inactive RNA-guided DNA binding protein, a protein comprising a DNA-binding zinc finger domain, a transcription activator-like effector (TALE), or any variant thereof.

84. The genetically engineered bacterium of embodiment 83, wherein the genetically engineered bacterium lacks (ii) and (iii), and the first target gene comprises: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s), and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase, and the agriculturally relevant compound is phosphate.

85. The genetically engineered bacterium of embodiment 83, wherein the genetically engineered bacterium further comprises (ii) and (iii), and the first target gene comprises: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase, and the agriculturally relevant compound is phosphate.

86. The genetically engineered bacterium of embodiment 83, wherein the genetically engineered bacterium further comprises (ii) and lacks (iii), and the first target gene is amtB, draT, glnA, glnB, glnK, glnZ, and/or nifL, and the agriculturally relevant compound is ammonia.

87. The genetically engineered bacterium of embodiment 83, wherein first target gene encodes a transcriptional activator protein and wherein the genetically engineered bacterium further comprises a control element comprising a promoter which is activated by the transcriptional activator protein and operably linked to at least one second target gene.

88. The genetically engineered bacterium of embodiment 83, wherein the genetically engineered bacterium comprises an even number of repressors and the first target gene comprises: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or morefix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), a pyrroloquinoline (PQQ) synthase, and the agriculturally relevant compound is phosphate.

89. The genetically engineered bacterium of embodiment 83, wherein the genetically engineered bacterium comprises an even number of repressors and the first target gene encodes an RNA sequence or protein comprising:
(a) a GlnE protein lacking an adenylyl removing domain which exhibits unidirectional adenylyltransferase (uAT) activity and the fertilizer is ammonia;
(b) a NifA protein, wherein the genetically engineered bacterium optionally comprises a loss-of-function mutation in any one or more of the amtB, draT, glnA, glnB, glnK, glnR, glnZ, or nifL genes, optionally wherein the genetically engineered bacterium comprises one or more heterologous genes from a wild-type or refactored nif or fix gene cluster and the fertilizer is ammonia;
(c) a GlnR protein and the fertilizer is ammonia;
(d) a glutaminase enzyme and the fertilizer is ammonia;
(e) a protein product of a refactored nif or fix gene cluster and the fertilizer is ammonia;
(f) a phytase enzyme and the agriculturally relevant compound is phosphate, optionally wherein the phytase enzyme comprises a cysteine phytase, a histidine acid phytase, or a beta-propeller phytase;
(g) an acid phosphatase enzyme and the agriculturally relevant compound is phosphate, optionally wherein the acid phosphatase enzyme is encoded by an acpA, aphA, phoC, napA, napD, or napE gene or variant thereof;
(h) a protein which stimulates organic acid release from the bacterium and the agriculturally relevant compound is phosphate, optionally wherein the protein which stimulates organic acid release comprises a gluconate dehydrogenase (GAD), a glucose dehydrogenase (GDH), or a pyrroloquinoline (PQQ) synthase;
(i) a repressor protein, wherein the repressor protein binds a natural or synthetic DNA motif in the promoter of any one or more second target gene(s) of the genetically engineered bacterium and inhibits expression of any one or more of the protein product(s) of the second target gene(s) and/or wherein the repressor protein optionally comprises the lambda repressor (cI), the tet repressor (TetR), the lac repressor (LacI), a catalytically inactive RNA-guided DNA binding protein, a protein comprising a DNA-binding zinc finger domain, a transcription activator-like effector (TALE), or any variant thereof and/or optionally wherein the second target gene(s) is/are a glnA, amtB, glnB, glnK, glnZ, nifL, and/or draT gene and the fertilizer is ammonia;
(j) a non-coding synthetic small RNA (sRNA); optionally wherein the non-coding synthetic small RNA (sRNA) binds a natural or synthetic DNA and/or RNA motif in the promoter, 5′ UTR, and/or coding region of any one or more second target gene(s) of the genetically engineered bacterium, optionally wherein the non-coding synthetic small RNA (sRNA) comprises a guide RNA that additionally binds an RNA-guided DNA endonuclease, RNA-guided RNA endonuclease, or variant thereof and/or optionally wherein the second target gene(s) is/are a glnA, amtB, glnB, glnK, glnZ, nifL, and/or draT gene and the fertilizer is ammonia;
(k) a site-specific recombinase (SSR) or integrase protein: wherein any one or more second target gene(s) of the genetically engineered bacterium and/or a promoter operatively linked thereto are flanked by site-specific recombinase recognition sites (SSRRS) in a direct configuration, optionally wherein the SSRRS comprise attL and attR sites and the genetically engineered bacterium comprises a gene encoding a recombinase directionality factor (RDF) and/or optionally wherein the second target gene(s) is/are a glnA, amtB, glnB, segment of glnE encoding an adenylyl-removing domain of a glutamine synthetase adenylyltransferase, glnK, glnZ, nifL, and/or draT gene and the fertilizer is ammonia;
(l) a site-specific recombinase (SSR) or integrase protein: wherein any one or more second target gene(s), the promoter(s) thereof, and/or the 5′ UTR(s) thereof of the genetically engineered bacterium comprise(s) one or more internal synthetic SSRRS and wherein the gene(s), promoter(s), the 5′ UTR(s), and/or a segment(s) thereof is/are excised or inactivated after a recombination event; optionally wherein the genetically engineered bacterium further comprises a plasmid comprising an SSRRS; optionally wherein the genetically engineered bacterium further comprises one or more genes encoding a recombinase directionality factor (RDF); and/or optionally wherein the second target gene(s) is/are a glnA, amtB, glnB, glnE, glnK, glnZ, nifL, and/or draT gene and the fertilizer is ammonia;
(m) a site-specific recombinase (SSR) or integrase protein, wherein any one or more second target gene(s), the promoter(s) thereof, and/or the 5′ UTR(s) thereof of the genetically engineered bacterium comprise(s) one or more internal integrative SSRRS and retain(s) activity of said second target gene(s), promoter(s), and/or 5′ UTR(s) and wherein the genetically engineered bacterium further comprises an integrative element comprising an SSRRS; optionally wherein the internal integrative SSRRS is/are an attB site(s), the integrative element comprises an SSRRS comprising an attP site; and/or optionally wherein the second target gene(s) is/are glnA, amtB, glnB, glnK, glnZ, nifL, and/or draT and the fertilizer is ammonia;
(n) a site-specific recombinase (SSR) or integrase protein, wherein a control element comprising a promoter and/or at least a segment of a 5′ UTR: (i) is flanked by site-specific recombinase recognition sites (SSRRS) in an inverted configuration; and (ii) is operably linked to one or more second target gene(s) of the genetically engineered bacterium; optionally wherein the control element which is operably linked to said second target gene(s) comprises a constitutive promoter, an inducible promoter, or a phosphate-sensitive promoter; and/or optionally wherein the second target gene(s) is/are glnA, amtB, glnB, glnK, glnZ, nifL, and/or draT and the fertilizer is ammonia;
(o) a site-specific recombinase (SSR) or integrase protein, wherein a control element comprising a promoter and at least a segment of a 5′ UTR: (i) is flanked by site-specific recombinase recognition sites (SSRRS) in an inverted configuration; and (ii) is operably linked to one or more second target gene(s) of the genetically engineered bacterium upon inversion by the SSR or integrase; optionally wherein the control element which is operably linked to said second target gene(s) upon inversion by the SSR or integrase comprises a constitutive promoter, an inducible promoter, or a phosphate-sensitive promoter; and/or optionally wherein the second target gene(s) is/are: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase, and the agriculturally relevant compound is phosphate;
(p) a site-specific DNA endonuclease, wherein any one or more second target gene(s) of the genetically engineered bacterium comprise(s) one or more specific DNA sequence(s) recognized by the site-specific DNA endonuclease, optionally wherein the site-specific DNA endonuclease comprises an RNA-guided DNA endonuclease, a protein comprising a DNA-binding zinc finger domain, a transcription activator-like effector (TALE), a meganuclease, a homing endonuclease, or a restriction endonuclease, and/or optionally wherein the second target gene(s) is/are glnA, amtB, glnB, glnK, glnZ, nifL, and/or draT and the fertilizer is ammonia;
(q) a protease which cleaves a protease specific recognition sequence (PSRS), wherein any one or more second target gene(s) of the genetically engineered bacterium comprise(s) one or more in-frame insertion(s) comprising DNA encoding the PSRS in the protein coding region of the second target gene(s), wherein the target protein product comprising the one or more in-frame insertion(s) has activity, and wherein cleavage of the target protein product(s) by the protease deactivates the target protein product(s), optionally wherein the location of the one or more in-frame insertion is given by Table 6, optionally wherein the second target gene is under the control of a constitutive promoter, and/or optionally wherein the second target gene(s) is/are glnA, amtB, glnB, glnK, glnZ, nifL, and/or draT and the fertilizer is ammonia;
(r) a first protein comprising a ClpAP ATP-dependent protease and a second protein comprising a ClpS Leu/N-recognin, wherein any one or more second target gene(s) of the genetically engineered bacterium encodes a third protein comprising an N-terminal-Leu, -Phe, -Trp, or -Tyr residue, and optionally wherein the first and second protein are operably linked to distinct control elements, and/or optionally wherein the second target gene(s) is/are glnA, amtB, glnB, glnK, glnZ, nifL, and/or draT and the fertilizer is ammonia;
(s) a protease which cleaves a protease specific recognition sequence (PSRS), wherein any one or more second target gene(s) of the genetically engineered bacterium comprise(s) an in-frame insertion(s) of DNA encoding the PSRS at the N-terminus of the protein coding region of the gene(s) followed by a -Leu, -Phe, -Trp, or -Tyr residue and wherein cleavage of PSRS from the N-terminus of the protein(s) encoded by the gene(s) by the protease results in a protein comprising an N-terminal-Leu, -Phe, -Trp, or -Tyr residue which is degraded by native ClpS and ClpAP, optionally wherein the second target gene(s) is/are glnA, amtB, glnB, glnK, glnZ, nifL, and/or draT and the fertilizer is ammonia;
(t) a transcriptional activator protein, wherein the transcriptional activator protein binds a natural or synthetic DNA motif in a promoter of a heterologous protease gene of the genetically engineered bacterium and activates expression of the protease, wherein any one or more second target gene(s) of the genetically engineered bacterium comprise(s) one or more in-frame insertion(s) of DNA encoding a PSRS, optionally wherein the transcriptional activator protein comprises a tet responsive element-binding tTA transcription factor or a transcriptional activator domain fused to a DNA targeting protein, optionally wherein the DNA targeting protein is a catalytically inactive RNA-guided DNA binding protein, a protein comprising a DNA-binding zinc finger domain, a transcription activator-like effector (TALE), or any variant thereof, optionally wherein the transcriptional activator domain is VP16, and/or optionally wherein the second target gene(s) is/are: glnA, amtB, glnB, glnK, glnZ, nifL, and/or draT and the fertilizer is ammonia;
(u) a repressor protein, wherein the repressor protein binds a natural or synthetic DNA motif in a promoter of a heterologous protease gene of the genetically engineered bacterium and inhibits expression of the protease, wherein any one or more second target gene(s) of the genetically engineered bacterium comprise(s) an in-frame insertion of DNA encoding an N-terminal amino acid tag designed to render the protein product(s) of the second target gene(s) inactive, followed by a PSRS, optionally wherein the repressor protein comprises the lambda repressor (cI), the tet repressor (TetR), the lac repressor (LacI), a catalytically inactive RNA-guided DNA binding protein, a protein comprising a DNA-binding zinc finger domain, a transcription activator-like effector (TALE), or any variant thereof, and/or optionally wherein the second target gene(s) is/are glnA, amtB, glnB, glnK, glnZ, nifL, and/or draT and the fertilizer is ammonia;
(v) a nanobody, wherein the nanobody binds a protein product of any one or more second target gene(s) of the genetically engineered bacterium, wherein binding of the nanobody to such protein product inhibits function of the protein product, optionally wherein the second target gene(s) is/are glnA, amtB, glnB, glnK, glnZ, nifL, and/or draT and the fertilizer is ammonia;
(w) an aptamer, wherein the aptamer binds a protein product of any one or more second target gene(s) of the genetically engineered bacterium and inhibits function of any one or more of the protein product(s) of the second target gene(s), optionally wherein the second target gene(s) is/are glnA, amtB, glnB, glnK, glnZ, nifL, and/or draT and the fertilizer is ammonia; or
(x) a transcriptional activator protein, wherein the transcriptional activator protein binds a natural or synthetic DNA motif in a promoter of any one or more second target gene(s) and increases expression of any one or more of the second target gene(s), optionally wherein the transcriptional activator protein comprises the tet responsive element-binding tTA transcription factor or a transcriptional activator domain fused to a DNA targeting protein, wherein the DNA targeting protein is optionally a catalytically inactive RNA-guided DNA binding protein, a protein comprising a DNA-binding zinc finger domain, a transcription activator-like effector (TALE), or any variant thereof, wherein the transcriptional activator domain is optionally VP16 and/or optionally wherein the second target gene(s) is/are any one or more of: (a) nifA, ntrC, glnR, a gene encoding a glutaminase enzyme, a gene encoding a GlnE protein lacking an adenylyl removing domain which exhibits unidirectional adenylyltransferase (uAT) activity, one or more rnif cluster gene(s), and/or one or more fix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, and/or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase and the agriculturally relevant compound is phosphate.

90. The genetically engineered bacterium of embodiment 83, wherein the genetically engineered bacterium comprises an odd number of repressors and the second target gene is amtB, draT, glnA, glnB, glnK, glnZ, and/or nifL, and the agriculturally relevant compound is ammonia.

91. The genetically engineered bacterium of any one of embodiments 1-73, wherein the protein of interest is a first transcriptional activator protein and wherein the genetically engineered bacterium further comprises:
(i) a second control element comprising a promoter which is activated by the first transcriptional activator protein and operably linked to a gene encoding a second transcriptional activator protein;
(ii) optionally a third control element comprising a promoter which is activated by the second transcriptional activator protein and operably linked to a gene encoding a third transcriptional activator protein;
(iii) optionally a fourth control element comprising a promoter which is activated by the third transcriptional activator protein and operably linked to a gene encoding a fourth transcriptional activator protein; and
(iv) a control element comprising a promoter which is activated by the second, third, or fourth transcriptional activator protein and which is operably linked to a first target gene, optionally wherein the first, second, third, and/or fourth transcriptional activator protein comprise(s) the tet responsive element-binding tTA transcription factor or an activator domain fused to a DNA targeting protein, optionally wherein the DNA targeting protein is a catalytically inactive RNA-guided DNA binding protein, a protein comprising a DNA-binding zinc finger domain, a transcription activator-like effector (TALE), and/or any variant thereof, optionally wherein the activator domain is VP16.

92. The genetically engineered bacterium of embodiment 91, wherein the first target gene comprises: (a) nifA, ntrC, glnR, a gene encoding a glutaminase enzyme, a gene encoding a GlnE protein lacking an adenylyl removing domain which exhibits unidirectional adenylyltransferase (uAT) activity, one or more nif cluster gene(s), and/or one or morefix cluster gene(s) and the agriculturally relevant compound is ammonia; or (b) a gene encoding a phytase enzyme, optionally wherein the phytase enzyme comprises a cysteine phytase, a histidine acid phytase, or a beta-propeller phytase, a gene encoding an acid phosphatase enzyme, optionally wherein the acid phosphatase is encoded by an acpA, aphA, phoC, napA, napD, or napE gene or variant thereof, or a gene encoding a protein which stimulates organic acid release from the bacterium, optionally wherein the protein which stimulates organic acid release comprises a gluconate dehydrogenase (GAD), a glucose dehydrogenase (GDH), or a pyrroloquinoline (PQQ) synthase, and the agriculturally relevant compound is phosphate.

93. The genetically engineered bacterium of any one of embodiments 1-73, wherein the protein of interest is a repressor protein and wherein the genetically engineered bacterium further comprises:
(i) a second control element comprising a promoter which is repressed by the repressor protein and operably linked to a gene encoding a transcriptional activator protein; and
(ii) a third control element comprising a promoter which is activated by the transcriptional activator protein and operably linked to a target gene.

94. The genetically engineered bacterium of embodiment 93, wherein the target gene is a glnA, amtB, glnB, glnK, glnZ, nifL, and/or draT gene and the agriculturally relevant compound is ammonia.

95. The genetically engineered bacterium of any one of embodiments 1-73, wherein the protein of interest is a transcriptional activator protein and wherein the genetically engineered bacterium further comprises:
(i) a second control element comprising a promoter which is activated by the transcriptional activator protein and operably linked to a gene encoding a repressor protein; and
(ii) a third control element comprising a promoter which is repressed by the repressor and operably linked to a target gene.

96. The genetically engineered bacterium of embodiment 95, wherein the target gene is a glnA, amtB, glnB, glnK, glnZ, nifL, and/or draT gene and the agriculturally relevant compound is ammonia.

97. The genetically engineered bacterium of embodiment 73, wherein the glnA, amtB, glnB, glnK, glnZ, nifL, and/or draT gene in (n), (o), (p), or (q) is under the control of a heterologous constitutive promoter.

98. The method of embodiment 89, wherein the glnA, amtB, glnB, glnK, glnZ, nifL, and/or draT gene in (q), (r), (s), or (t) is under the control of a heterologous constitutive promoter.

99. The genetically engineered bacterium of any one of embodiments 1-72, wherein the plant nutrient is phosphate and the protein of interest operably linked to the control element comprises:
a. a phytase enzyme, optionally wherein the phytase enzyme comprises a cysteine phytase, a histidine acid phytase, or a beta-propeller phytase and/or optionally wherein the phytase enzyme comprises a protein sequence having at least 70%, 75%, 80%, 85%, 90%, 95%, 98%, or 99% sequence identity to SEQ ID NO: 58-138, or 139;
b. an acid phosphatase enzyme, optionally wherein the acid phosphatase is encoded by an acpA, aphA, phoC, napA, napD, or napE gene or variant thereof;
c. a protein which stimulates organic acid release from the bacterium, optionally wherein the protein comprises a gluconate dehydrogenase (GAD), a glucose dehydrogenase (GDH), a pyrroloquinoline (PQQ) synthase including pqqFABCDEG, or any combination of GAD, GDH, and PQQ; or, d. any combination of proteins of a, b, or c.

100. The genetically engineered bacterium of any one of embodiments 58 to 99, wherein: (i) the genetically engineered bacterium comprises the heterologous gene expression cassette in a bacterium originally isolated from a plant growth medium or a plant; and (ii) wherein the bacterium originally isolated from the plant growth medium or the plant lacks the heterologous gene expression cassette.

101. The genetically engineered bacterium of any one of embodiments 58 to 100, wherein the bacterium is selected from the group of gram-negative bacteria.

102. The genetically engineered bacterium of any one of embodiments 58 to 100, wherein the bacterium is selected from the groups of bacteria of the taxonomic classes of alphaproteobacteria, betaproteobacteria, and gammaproteobacteria.

103. The genetically engineered bacterium of any one of embodiments 58 to 100, wherein the bacterium is selected from the groups of bacteria in the taxonomic genera of Acetobacter, Acidothermus, Acinetobacter, Agrobacterium, Aromatoleum, Arthrobacter, Azoarcus, Azorhizobium, Azospirillum, Azotobacter, Bacillus, Bifidobacterium, Bradyrhizobium, Burkholderia, Conexibacter, Curtobacterium, Ensifer, Enterobacter, Erwinia, Escherichia, Flavobacterium, Frankia, Gaiella, Gluconacetobacter, Gluconobacter, Herbaspirillum, Klebsiella, Kosakonia, Lactobacillus, Lactococcus, Lysinibacillus, Maritimibacter, Methylobacterium, Nitrosocosmicus, Nitrososphaera, Paenarthrobacter, Paenibacillus, Panotea, Pediococcus, Peribacillus, Phytobacter, Priestia, Pseudarthrobacter, Pseudomonas, Rahnella, Rhizobium, Rhodococcus, Rhodoplanes, Rhodopseudomonas, Rhodospirillum, Serratia, Solirubrobacter, Sphingobacterium, Sphingomonas, Stenotrophomonas, Streptomyces, Stutzerimonas, Variovorax, Xanthobacter, and Yoonia, optionally wherein the bacteria are selected from at least one of the taxonomic genera selected from the group consisting of Herbaspirillum, Azospirillum, Kosakonia, Pseudomonas, Enterobacter, Klebsiella, Paenibacillus, Phytobacter, Rahnella, Sphingomonas, or Variovorax.

104. A composition comprising the genetically engineered bacterium of any one of any one of embodiments 58 to 103 and an agriculturally acceptable carrier.

105. The composition of embodiment 104, wherein the composition further comprises:
(i) an agriculturally acceptable adjuvant, optionally wherein the adjuvant comprises an adhesive agent, a desiccant, and/or a dispersant;
(ii) a fungicide, an insecticide, a nematicide, a rodenticide, and/or a bacteriocide; and/or
(iii) a fertilizer, optionally wherein the fertilizer comprises nitrogen, phosphorous, potassium, calcium, sulfur, magnesium, boron, chloride, manganese, iron, zinc, copper, molybdenum, and/or selenium.

106. The composition of embodiment 104 or 105, wherein the composition is in a solid form, optionally wherein the solid form comprises a wettable powder, granules, a gel, pellets, or microencapsulated particles.

107. The composition of embodiment 104, wherein the composition is in a liquid form, optionally wherein the liquid form comprises an aqueous solution, aqueous suspension, water-in-oil emulsion, an oil, or an alcohol.

108. A plant part or plant propagule which is at least partially coated, imbibed, or mixed with the composition of embodiment 104.

109. The plant part of embodiment 108, wherein the part is a leaf, stem, root, or seed.

110. The plant propagule of embodiment 108, wherein the propagule comprises a cutting, tuber, or stolon.

111. Use of the plant part or plant propagule of any one of embodiments 108-110 to grow a crop.

112. The use of embodiment 111, wherein fertilizer input is reduced in comparison to a crop grown from a plant part or plant propagule which has not been at least partially coated, imbibed, or mixed with the composition.

113. An agricultural system comprising:
(i) at least one engineered bacterium of any one of embodiments 58 to 103;
(ii) at least one plant growth medium; and
(iii) at least one crop plant, crop plant seed, or crop plant vegetative propagule; wherein the plant growth medium, crop plant, crop seed, and/or crop plant propagule comprise, are at least partially coated, imbibed, and/or are mixed with the engineered bacterium or a composition comprising the engineered bacterium and an agriculturally acceptable carrier.

114. The system of embodiment 113, wherein the crop plant, seed, or vegetative propagule is an alfalfa, apple, banana, barley, bean, buckwheat, cabbage, cassava, chili, clover, coffee, corn, cotton, cowpea, cucumber, fonio, garlic, herb, lettuce, maize, melon, millet, nut, oat, oilseed rape, olive, onion, orange, sunflower, pea, Phaseolus bean, plantain, potato, quinoa, rice, rye, safflower, sorghum, soybean, sugar beet, sugar cane, sunflower, tangerine, tobacco, tomato, triticale, turnip, wheat, or yam plant, seed, or vegetative propagule.

115. The system of embodiment 113 or 114, wherein the plant growth medium comprises soil and/or water, optionally wherein the soil and/or water is non-axenic.

116. The system of embodiment 113, wherein the vegetative propagule comprises a cutting, tuber, or stolon.

117. A treated plant seed or plant propagule system comprising:
(i) at least one crop plant seed or crop plant vegetative propagule; and
(ii) at least one engineered bacterium of any one of embodiments 58 to 103,
   wherein the crop plant seed or crop plant propagule are at least partially coated, imbibed, and/or mixed with the engineered bacterium or a composition comprising the engineered bacterium and an agriculturally acceptable carrier.

118. The system of embodiment 117, wherein the crop plant, seed, or vegetative propagule is an alfalfa, apple, banana, barley, bean, buckwheat, cabbage, cassava, chili, clover, coffee, corn, cotton, cowpea, cucumber, fonio, garlic, herb, lettuce, maize, melon, millet, nut, oat, oilseed rape, olive, onion, orange, sunflower, pea, Phaseolus bean, plantain, potato, quinoa, rice, rye, safflower, sorghum, soybean, sugar beet, sugar cane, sunflower, tangerine, tobacco, tomato, triticale, turnip, wheat, or yam plant, seed, or vegetative propagule.

119. The system of embodiment 117 or 118, wherein the vegetative propagule comprises a cutting, tuber, or stolon.

120. A genetically engineered bacterium comprising: (i) a first recombinant DNA molecule comprising a phosphate-sensitive promoter of a bacterial pstS phosphate ABC transporter gene operably linked to DNA encoding a transcript comprising a ribosome binding site (RBS) operably linked to a transcriptional repressor protein coding region, wherein the RBS, when substituted in place of nucleotides 723 to 810 of control green fluorescent protein (GFP) reporter gene expression cassette of SEQ ID NO: 522, provides 0.1% to 44% of operably linked GFP reporter gene expression provided by the control GFP reporter gene expression cassette of SEQ ID NO: 522 when expressed under otherwise identical conditions; and (ii) a second recombinant DNA molecule comprising a promoter which is repressed by the transcriptional repressor protein and which is operably linked to DNA encoding a bacterial glutamine synthetase, wherein the genetically engineered bacterium is of the genus Kosakonia.

121. The bacterium of embodiment 120, wherein the bacterial pstS phosphate ABC transporter gene is a Klebsiella sp., Klebsiella variicola, Kosakonia sp., or Kosakonia sacchari pstS phosphate ABC transporter gene.

122. The bacterium of embodiment 120 or 121, wherein the bacterial pstS phosphate ABC transporter gene encodes a phosphate ABC transporter protein having at least 76%, 80%, 85%, 90%, 95%, 98%, or 99% sequence identity to SEQ ID NO: 513 and comprising the polypeptide of SEQ ID NO: 514.

123. The bacterium of any one of embodiments 1 to 119, 120, or 121, wherein the phosphate-sensitive promoter comprises a sequence having at least 76%, 80%, 85%, 90%, 95%, 98%, or 99% 95% sequence identity to SEQ ID NO: 515 and comprising at least one Pho box comprising the DNA molecule of SEQ ID NO: 525, 526, 527, 528, and/or 529.

124. The bacterium of any one of embodiments 120 to 123, wherein the transcriptional repressor protein is a bacterial TetR protein.

125. The bacterium of any one of embodiments 120 to 124, wherein the ribosome binding site (RBS) which is operably linked to a transcriptional repressor protein coding region comprises a BCD22vL ribosome binding site encoded by SEQ ID NO: 449.

126. The bacterium of any one of embodiments 120 to 125, wherein transcriptional repressor protein coding region encodes a bacterial TetR transcriptional repressor protein having at least 80%, 85%, 90%, 95%, 98%, or 99% sequence identity to SEQ ID NO: 516.

127. The bacterium of embodiment 126, wherein transcriptional repressor protein coding region encodes a bacterial TetR transcriptional repressor protein comprising the amino acid sequence of SEQ ID NO: 516.

128. The bacterium of any one of embodiments 120 to 127, wherein the bacterial glutamine synthetase is an Azospirillum glutamine synthetase and the endogenous gene encoding the Kosakonia glutamine synthetase protein contains a null mutation or is deleted.

129. The bacterium of any one of embodiments 120 to 127, wherein the bacterial glutamine synthetase comprises a Kosakonia glutamine synthetase.

130. The bacterium of embodiment 129, wherein the Kosakonia glutamine synthetase is encoded by the endogenous Kosakonia gene and wherein the endogenous Kosakonia gene is operably linked to the promoter which is repressed by the TetR repressor protein.

131. The bacterium of embodiment 120, wherein: (i) the first recombinant DNA molecule comprises the Klebsiella variicola pstS phosphate-sensitive promoter having at least 80%, 85%, 90%, 95%, 98%, or 99% sequence identity to SEQ ID NO: 515, the BCD22vL ribosome binding site (RBS) of SEQ ID NO: 449, and the transcriptional repressor protein comprising the amino acid sequence having at least 95% sequence identity to SEQ ID NO: 516; and (ii) the second recombinant DNA molecule comprises the Ptet promoter having at least 80%, 85%, 90%, 95%, 98%, or 99% sequence identity to SEQ ID NO: 32, and the bacterial glutamine synthetase is a glutamine synthetase having at least 80%, 85%, 90%, 95%, 98%, or 99% sequence identity to SEQ ID NO: 229.

132. A composition comprising the genetically engineered bacterium of any one of embodiments 120 to 131 and an agriculturally acceptable carrier.

133. The composition of embodiment 132, wherein the composition further comprises: (i) an agriculturally acceptable adjuvant, optionally wherein the adjuvant comprises an adhesive agent, a desiccant, and/or a dispersant; (ii) a fungicide, an insecticide, a nematicide, a rodenticide, and/or a bacteriocide; and/or (iii) a fertilizer, optionally wherein the fertilizer comprises nitrogen, phosphorous, potassium, calcium, sulfur, magnesium, boron, chloride, manganese, iron, zinc, copper, molybdenum, and/or selenium.

134. A plant part or plant propagule which is at least partially coated, imbibed, or mixed with the composition of embodiment 132 or 133.

135. A method of providing nitrogen to a plant comprising placing the one genetically engineered bacterium of any one of embodiments 120 to 131 or the composition of embodiment 132 or 133 into a plant growth medium, wherein a decrease in phosphate concentration in the plant growth medium results in the production of ammonia which is provided to the plant when grown in the growth medium.

136. The method of embodiment 135, wherein the plant growth medium comprises soil and/or water, optionally wherein the soil and/or water is non-axenic.

137. The method of embodiment 135 or 136, wherein the placing is prior to, during, and/or after depositing a seed or vegetative propagule in the plant growth medium.

138. The method of any one of embodiments 135 to 137, wherein the plant growth medium has been treated by adding inorganic phosphate to the plant growth medium at a rate of about 3 g/m² to about 15 g/m².

139. The method of any one of embodiments 135 to 138, further comprising: (i) determining leaf nitrogen and/or chlorophyll concentrations in a plant grown in the plant growth medium; and (ii) placing or re-applying the genetically engineered bacterium into the plant growth medium and/or in contact with the plant when the leaf nitrogen and/or chlorophyll concentrations in the plant are sub-optimal for yield.

140. An agricultural system comprising: (i) at least one engineered bacterium of any one of embodiments 120 to 131; (ii) at least one plant growth medium; and (iii) at least one crop plant, crop plant seed, or crop plant vegetative propagule; wherein the plant growth medium, crop plant, crop seed, and/or crop plant propagule comprise, are at least partially coated, imbibed, and/or are mixed with the engineered bacterium or a composition comprising the engineered bacterium and an agriculturally acceptable carrier.

141. The system of embodiment 140, wherein the crop plant, seed, or vegetative propagule is an alfalfa, apple, banana, barley, bean, buckwheat, cabbage, cassava, chili, clover, coffee, corn, cotton, cowpea, cucumber, fonio, garlic, herb, lettuce, maize, melon, millet, nut, oat, oilseed rape, olive, onion, orange, sunflower, pea, Phaseolus bean, plantain, potato, quinoa, rice, rye, safflower, sorghum, soybean, sugar beet, sugar cane, sunflower, tangerine, tobacco, tomato, triticale, turnip, wheat, or yam plant, seed, or vegetative propagule.

142. The system of embodiment 140 or 141, wherein the plant growth medium comprises soil and/or water, optionally wherein the soil and/or water is non-axenic.

143. The system of any one of embodiments 140 to 142, wherein the vegetative propagule comprises a cutting, tuber, or stolon.

144. A treated plant seed or plant propagule system comprising: (i) at least one crop plant seed or crop plant vegetative propagule; and (ii) at least one engineered bacterium of any one of embodiments 120 to 131, wherein the crop plant seed or crop plant propagule are at least partially coated, imbibed, and/or mixed with the engineered bacterium or a composition comprising the engineered bacterium and an agriculturally acceptable carrier.

145. The system of embodiment 144, wherein the crop plant, seed, or vegetative propagule is an alfalfa, apple, banana, barley, bean, buckwheat, cabbage, cassava, chili, clover, coffee, corn, cotton, cowpea, cucumber, fonio, garlic, herb, lettuce, maize, melon, millet, nut, oat, oilseed rape, olive, onion, orange, sunflower, pea, Phaseolus bean, plantain, potato, quinoa, rice, rye, safflower, sorghum, soybean, sugar beet, sugar cane, sunflower, tangerine, tobacco, tomato, triticale, turnip, wheat, or yam plant, seed, or vegetative propagule.

In the description, tables, and numbered embodiments 1-145 set forth herein, genes and the proteins they encode which are referred to solely by name include genes and proteins identified in Table 5 and the sequence listing, include genes having at least 70%, 75%, 76%, 80%, 85%, 90%, 95%, 98%, or 99% identity to the genes identified in Table 5 and the sequence listing, and include proteins having at least 70%, 75%, 80%, 85%, 90%, 95%, 98%, or 99% identity to the proteins identified in Table 5 and the sequence listing. GlnE proteins lacking an adenylyl removing domain which exhibit unidirectional adenylyltransferase (uAT) activity referred to solely by name in the preceding description, tables, and numbered embodiments include proteins comprising a protein sequence having at least 70%, 80%, 85%, 90%, 95%, 98%, or 99% identity to SEQ ID NO: 16-24, or 25. Genes encoding GlnE proteins lacking an adenylyl removing domain which exhibit unidirectional adenylyltransferase (uAT) activity referred to solely by name in the preceding description, tables, and numbered embodiments include genes comprising a DNA sequence having at least 70%, 80%, 85%, 90%, 95%, 98%, or 99% identity to SEQ ID NO: 1-14, or 15. Wild-type GlnA proteins (glutamine synthetase or GS proteins) referred to solely by name in the preceding description, tables, and numbered embodiments include proteins having a protein sequence having at least 70%, 80%, 85%, 90%, 95%, 98%, or 99% identity to SEQ ID NO: 229, 230, 460-512, 519-521, 530-532, or 533. Wild-type glnA genes encoding wild-type GlnA proteins referred to solely by name in the preceding description, tables, and numbered embodiments include glnA genes encoding proteins having a protein sequence having at least 70%, 80%, 85%, 90%, 95%, 98%, or 99% identity to SEQ ID NO: 229, 230, 460-512, 519-521, 530-532, or 533. Phytases referred to solely by name in the preceding description, tables, and numbered embodiments include phytases comprising a protein sequence having at least 70%, 75%, 80%, 85%, 90%, 95%, 98%, or 99% sequence identity to SEQ ID NO: 58-138, or 139. Phytase or phy genes referred to solely by name in the preceding description, tables, and numbered embodiments include phytase genes encoding phytase proteins comprising a protein sequence having at least 70%, 75%, 80%, 85%, 90%, 95%, 98%, or 99% sequence identity to SEQ ID NO: 58-138, or 139.

TABLE 5
Summary of Biological Sequences in Sequence Listing

| | SEQ | | |
|---|---|---|---|
| Description | ID NO | Type | Organism name |
| E. coli uAT10 gene (glnE 423-946) | 1 | DNA | synthetic construct |
| uAT10r2 gene (Codon refactored uAT10 for A. | 2 | DNA | synthetic construct |
| brasilense) | | | |
| uAT10r3 gene (Codon refactored uAT10 for A. | 3 | DNA | synthetic construct |
| brasilense) | | | |
| uAT10r4 gene (Codon refactored uAT10 for A. | 4 | DNA | synthetic construct |
| brasilense) | | | |
| uAT10r5 gene (Codon refactored uAT10 for A. | 5 | DNA | synthetic construct |
| brasilense) | | | |
| E. coli uAT11 gene (glnE 609-946) | 6 | DNA | synthetic construct |
| A. brasilense uAT21 gene (glnE 449-1003) | 7 | DNA | synthetic construct |
| A. brasilense uAT22 gene (glnE 481-1003) | 8 | DNA | synthetic construct |
| A. brasilense uAT23 gene (glnE 506-1003) | 9 | DNA | synthetic construct |
| A. brasilense uAT24 gene (glnE 529-1003) | 10 | DNA | synthetic construct |
| A. brasilense uAT25 gene (glnE 625-1003) | 11 | DNA | synthetic construct |
| uAT23r2 gene (Codon refactored uAT23 gene) | 12 | DNA | synthetic construct |
| A. caulinodans uAT-Acl gene (glnE 528-996) | 13 | DNA | synthetic construct |
| A. caulinodans uAT-Ac2 gene (glnE 538-996) | 14 | DNA | synthetic construct |
| A. caulinodans uAT-Ac3 gene (glnE 548-996) | 15 | DNA | synthetic construct |
| E. coli uAT10 protein (GlnE 423-946) | 16 | PRT | synthetic construct |
| E. coli uAT11 protein (GlnE 609-946) | 17 | PRT | synthetic construct |
| A. brasilense uAT21 protein (GlnE 449-1003) | 18 | PRT | synthetic construct |
| A. brasilense uAT22 protein (GlnE 481-1003) | 19 | PRT | synthetic construct |
| A. brasilense uAT23 protein (GlnE 506-1003) | 20 | PRT | synthetic construct |
| A. brasilense uAT24 protein (GlnE 529-1003) | 21 | PRT | synthetic construct |
| A. brasilense uAT25 protein (GlnE 625-1003) | 22 | PRT | synthetic construct |
| A. caulinodans uAT-Ac1 protein (GlnE 528-996) | 23 | PRT | synthetic construct |
| A. caulinodans uAT-Ac2 protein (GlnE 538-996) | 24 | PRT | synthetic construct |
| A. caulinodans uAT-Ac3 protein (GlnE 548-996) | 25 | PRT | synthetic construct |
| Lambda repressor protein (cI) | 26 | PRT | synthetic construct |
| Tet repressor protein (TetR) | 27 | PRT | synthetic construct |
| Reverse tet repressor protein (rTetR) | 28 | PRT | synthetic construct |
| Lac repressor protein (LacI) | 29 | PRT | synthetic construct |
| PhIF repressor protein (PhlF) | 30 | PRT | synthetic construct |
| pR promoter (binds cl repressor) | 31 | DNA | synthetic construct |
| Ptet promoter (binds TetR repressors) | 32 | DNA | synthetic construct |
| Plac promoter (binds lac repressor) | 33 | DNA | synthetic construct |
| PphlA promoter (binds PhIF repressor) | 34 | DNA | synthetic construct |
| PhiC31 integrase protein | 35 | PRT | synthetic construct |
| IntM integrase protein | 36 | PRT | synthetic construct |
| IntS integrase protein | 37 | PRT | synthetic construct |
| IntG integrase protein | 38 | PRT | synthetic construct |
| YdcL integrase protein | 39 | PRT | synthetic construct |
| Sxt/R391 integrase protein | 40 | PRT | synthetic construct |
| PhiC31 integrase gene | 41 | DNA | synthetic construct |
| PhiC31 integrase attP recombinase recognition site | 42 | DNA | synthetic construct |
| PhiC31 integrase attB recombinase recognition site | 43 | DNA | synthetic construct |
| J23104 constitutive promoter | 44 | DNA | synthetic construct |
| Tobacco etch virus (TEV) protease (P04517) | 45 | PRT | synthetic construct |
| Tobacco vein mottling virus (TVMV) protease (P09814) | 46 | PRT | synthetic construct |
| Sunflower mild mosaic virus (SMMV) protease | 47 | PRT | synthetic construct |
| Turnip mosaic virus (TuMV) protease (Q02597) | 48 | PRT | synthetic construct |
| Soybean mosaic virus (SMV) protease (Q90069) | 49 | PRT | synthetic construct |
| Plum pox potyvirus (PPV) protease (P17767) | 50 | PRT | synthetic construct |
| Hepatitis C virus (HCV) protease (P27958) | 51 | PRT | synthetic construct |
| Coagulation factor Xa (P00742) | 52 | PRT | Homo sapiens |
| Furin (P09958) | 53 | PRT | Homo sapiens |
| TEV protease coding sequence optimized for A. | 54 | DNA | synthetic construct |
| brasilense | | | |
| TVMV protease coding sequence optimized for A. | 55 | DNA | synthetic construct |
| brasilense | | | |
| SMMV protease coding sequence optimized for A. | 56 | DNA | synthetic construct |
| brasilense | | | |
| B. subtilis phytase protein (PhyC) | 57 | PRT | Bacillus subtilis |
| Phytase protein (HAP) | 58 | PRT | unidentified |
| Phytase protein (BPP) | 59 | PRT | unidentified |
| Phytase protein (CPhy) | 60 | PRT | Clostridium colicanis |
| Phytase protein (BPP) | 61 | PRT | Chlorobaculum |
| | | | limnaeum |
| Phytase protein (BPP) | 62 | PRT | Bacillus |
| | | | amyloliquefaciens |
| Phytase protein (CPhy) | 63 | PRT | Cystobacter fuscus |
| Phytase protein (HAP) | 64 | PRT | Acinetobacter baumannii |
| Phytase protein (CPhy) | 65 | PRT | unidentified |
| Phytase protein (CPhy) | 66 | PRT | Clostridium ihumii |
| Phytase protein (CPhy) | 67 | PRT | Desulfovibrio |
| | | | fructosovorans |
| Phytase protein (CPhy) | 68 | PRT | Clostridium novyi |
| Phytase protein (CPhy) | 69 | PRT | Clostridium bornimense |
| Phytase protein (HAP) | 70 | PRT | Bacteroides |
| | | | stercorirosoris |
| Phytase protein (CPhy) | 71 | PRT | Clostridium |
| | | | paraputrificum |
| Phytase protein (CPhy) | 72 | PRT | Clostridium |
| | | | pasteurianum |
| Phytase protein (CPhy) | 73 | PRT | Clostridium perfringens |
| Phytase protein (CPhy) | 74 | PRT | Clostridium botulinum |
| Phytase protein (CPhy) | 75 | PRT | unidentified |
| Phytase protein (CPhy) | 76 | PRT | Acidovorax avenae |
| Phytase protein (CPhy) | 77 | PRT | Clostridium sp. |
| Phytase protein (CPhy) | 78 | PRT | Clostridium sp. |
| Phytase protein (CPhy) | 79 | PRT | Acidaminococcus sp. |
| Phytase protein (CPhy) | 80 | PRT | Anaerovibrio lipolyticus |
| Phytase protein (BPP) | 81 | PRT | Cellulophaga sp. |
| Phytase protein (HAP) | 82 | PRT | Bifidobacterium dentium |
| Phytase protein (BPP) | 83 | PRT | Chryseobacterium sp. |
| Phytase protein (BPP) | 84 | PRT | Erythrobacter litoralis |
| Phytase protein (BPP) | 85 | PRT | unidentified |
| Phytase protein (BPP) | 86 | PRT | Devosia riboflavina |
| Phytase protein (CPhy) | 87 | PRT | Bradyrhizobium sp. |
| Phytase protein (BPP) | 88 | PRT | Algoriphagus marincola |
| Phytase protein (HAP) | 89 | PRT | Bryobacter aggregatus |
| Phytase protein (BPP) | 90 | PRT | Asticcacaulis sp. |
| Phytase protein (BPP) | 91 | PRT | unidentified |
| Phytase protein (BPP) | 92 | PRT | Bacillus licheniformis |
| Phytase protein (BPP) | 93 | PRT | Deinococcus |
| | | | proteolyticus |
| Phytase protein (HAP) | 94 | PRT | unidentified |
| Phytase protein (BPP) | 95 | PRT | Bacillus sp. |
| Phytase protein (BPP) | 96 | PRT | Saccharicrinis |
| | | | fermentans |
| Phytase protein (HAP) | 97 | PRT | Asticcacaulis |
| | | | benevestitus |
| Phytase protein (BPP) | 98 | PRT | Amycolatopsis orientalis |
| Phytase protein (HAP) | 99 | PRT | Asticcacaulis |
| | | | benevestitus |
| Phytase protein (HAP) | 100 | PRT | Acidobacteriaceae sp. |
| Phytase protein (HAP) | 101 | PRT | Bacteroides sp. |
| Phytase protein (HAP) | 102 | PRT | Barnesiella |
| | | | intestinihominis |
| Phytase protein (HAP) | 103 | PRT | Endozoicomonas |
| | | | elysicola |
| Phytase protein (HAP) | 104 | PRT | Asaia astilbes |
| Phytase protein (HAP) | 105 | PRT | Alistipes timonensis |
| Phytase protein (HAP) | 106 | PRT | unidentified |
| Phytase protein (HAP) | 107 | PRT | unidentified |
| Phytase protein (HAP) | 108 | PRT | Desulfovibrio piger |
| Phytase protein (BPP) | 109 | PRT | Chitinibacter sp. |
| Phytase protein (BPP) | 110 | PRT | Caulobacter sp. |
| Phytase protein (HAP) | 111 | PRT | Butyrivibrio sp. |
| Phytase protein (HAP) | 112 | PRT | Desulfatirhabdium |
| | | | butyrativorans |
| Phytase protein (BPP) | 113 | PRT | Cystobacter fuscus |
| Phytase protein (BPP) | 114 | PRT | unidentified |
| Phytase protein (BPP) | 115 | PRT | Microbulbifer |
| | | | agarilyticus |
| Phytase protein (BPP) | 116 | PRT | Acinetobacter nectaris |
| Phytase protein (BPP) | 117 | PRT | Catenovulum |
| | | | agarivorans |
| Phytase protein (HAP) | 118 | PRT | Corynebacterium |
| | | | timonense |
| Phytase protein (BPP) | 119 | PRT | Alteromonas macleodii |
| Phytase protein (CPhy) | 120 | PRT | Clostridium sp. |
| Phytase protein (CPhy) | 121 | PRT | Clostridium beijerinckii |
| Phytase protein (CPhy) | 122 | PRT | Bdellovibrio |
| | | | bacteriovorus |
| Phytase protein (CPhy) | 123 | PRT | Clostridium |
| | | | tyrobutyricum |
| Phytase protein (CPhy) | 124 | PRT | Acidaminococcus |
| | | | fermentans |
| Phytase protein (CPhy) | 125 | PRT | Desulfovibrio |
| | | | alcoholivorans |
| Phytase protein (CPhy) | 126 | PRT | Desulfovibrio frigidus |
| Phytase protein (CPhy) | 127 | PRT | Clostridium |
| | | | acetobutylicum |
| Phytase protein (CPhy) | 128 | PRT | Allisonella |
| | | | histaminiformans |
| Phytase protein (CPhy) | 129 | PRT | Centipeda periodontii |
| Phytase protein (CPhy) | 130 | PRT | Desulfovibrio |
| | | | magneticus |
| Phytase protein (HAP) | 131 | PRT | Burkholderiales |
| | | | bacterium |
| Phytase protein (HAP) | 132 | PRT | Acinetobacter nectaris |
| Phytase protein (HAP) | 133 | PRT | Dyella marensis |
| Phytase protein (HAP) | 134 | PRT | Escherichia coli |
| Phytase protein (HAP) | 135 | PRT | Anaerovibrio sp. |
| Phytase protein (HAP) | 136 | PRT | Avibacterium |
| | | | paragallinarum |
| Phytase protein (HAP) | 137 | PRT | Cronobacter sakazakii |
| Phytase protein (HAP) | 138 | PRT | unidentified |
| Phytase protein (HAP) | 139 | PRT | Corynebacterium |
| | | | singulare |
| B. subtilis phytase coding sequence (phyC) | 140 | DNA | Bacillus subtilis |
| Phytase coding sequence (HAP) | 141 | DNA | unidentified |
| Phytase coding sequence (BPP) | 142 | DNA | unidentified |
| Phytase coding sequence (CPhy) | 143 | DNA | Clostridium colicanis |
| Phytase coding sequence (BPP) | 144 | DNA | Chlorobaculum |
| | | | limnaeum |
| Phytase coding sequence (BPP) | 145 | DNA | Bacillus |
| | | | amyloliquefaciens |
| Phytase coding sequence (CPhy) | 146 | DNA | Cystobacter fuscus |
| Phytase coding sequence (HAP) | 147 | DNA | Acinetobacter baumannii |
| Phytase coding sequence (CPhy) | 148 | DNA | unidentified |
| Phytase coding sequence (CPhy) | 149 | DNA | Clostridium ihumii |
| Phytase coding sequence (CPhy) | 150 | DNA | Desulfovibrio |
| | | | fructosovorans |
| Phytase coding sequence (CPhy) | 151 | DNA | Clostridium novyi |
| Phytase coding sequence (CPhy) | 152 | DNA | Clostridium bornimense |
| Phytase coding sequence (HAP) | 153 | DNA | Bacteroides |
| | | | stercorirosoris |
| Phytase coding sequence (CPhy) | 154 | DNA | Clostridium |
| | | | paraputrificum |
| Phytase coding sequence (CPhy) | 155 | DNA | Clostridium |
| | | | pasteurianum |
| Phytase coding sequence (CPhy) | 156 | DNA | Clostridium perfringens |
| Phytase coding sequence (CPhy) | 157 | DNA | Clostridium botulinum |
| Phytase coding sequence (CPhy) | 158 | DNA | unidentified |
| Phytase coding sequence (CPhy) | 159 | DNA | Acidovorax avenae |
| Phytase coding sequence (CPhy) | 160 | DNA | Clostridium sp. |
| Phytase coding sequence (CPhy) | 161 | DNA | Clostridium sp. |
| Phytase coding sequence (CPhy) | 162 | DNA | Acidaminococcus sp. |
| Phytase coding sequence (CPhy) | 163 | DNA | Anaerovibrio lipolyticus |
| Phytase coding sequence (BPP) | 164 | DNA | Cellulophaga sp. |
| Phytase coding sequence (HAP) | 165 | DNA | Bifidobacterium dentium |
| Phytase coding sequence (BPP) | 166 | DNA | Chryseobacterium sp. |
| Phytase coding sequence (BPP) | 167 | DNA | Erythrobacter litoralis |
| Phytase coding sequence (BPP) | 168 | DNA | unidentified |
| Phytase coding sequence (BPP) | 169 | DNA | Devosia riboflavina |
| Phytase coding sequence (CPhy) | 170 | DNA | Bradyrhizobium sp. |
| Phytase coding sequence (BPP) | 171 | DNA | Algoriphagus marincola |
| Phytase coding sequence (HAP) | 172 | DNA | Bryobacter aggregatus |
| Phytase coding sequence (BPP) | 173 | DNA | Asticcacaulis sp. |
| Phytase coding sequence (BPP) | 174 | DNA | unidentified |
| Phytase coding sequence (BPP) | 175 | DNA | Bacillus licheniformis |
| Phytase coding sequence (BPP) | 176 | DNA | Deinococcus |
| | | | proteolyticus |
| Phytase coding sequence (HAP) | 177 | DNA | unidentified |
| Phytase coding sequence (BPP) | 178 | DNA | Bacillus sp. |
| Phytase coding sequence (BPP) | 179 | DNA | Saccharicrinis |
| | | | fermentans |
| Phytase coding sequence (HAP) | 180 | DNA | Asticcacaulis |
| | | | benevestitus |
| Phytase coding sequence (BPP) | 181 | DNA | Amycolatopsis orientalis |
| Phytase coding sequence (HAP) | 182 | DNA | Asticcacaulis |
| | | | benevestitus |
| Phytase coding sequence (HAP) | 183 | DNA | Acidobacteriaceae sp. |
| Phytase coding sequence (HAP) | 184 | DNA | Bacteroides sp. |
| Phytase coding sequence (HAP) | 185 | DNA | Barnesiella |
| | | | intestinihominis |
| Phytase coding sequence (HAP) | 186 | DNA | Endozoicomonas |
| | | | elysicola |
| Phytase coding sequence (HAP) | 187 | DNA | Asaia astilbes |
| Phytase coding sequence (HAP) | 188 | DNA | Alistipes timonensis |
| Phytase coding sequence (HAP) | 189 | DNA | unidentified |
| Phytase coding sequence (HAP) | 190 | DNA | unidentified |
| Phytase coding sequence (HAP) | 191 | DNA | Desulfovibrio piger |
| Phytase coding sequence (BPP) | 192 | DNA | Chitinibacter sp. |
| Phytase coding sequence (BPP) | 193 | DNA | Caulobacter sp. |
| Phytase coding sequence (HAP) | 194 | DNA | Butyrivibrio sp. |
| Phytase coding sequence (HAP) | 195 | DNA | Desulfatirhabdium |
| | | | butyrativorans |
| Phytase coding sequence (BPP) | 196 | DNA | Cystobacter fuscus |
| Phytase coding sequence (BPP) | 197 | DNA | unidentified |
| Phytase coding sequence (BPP) | 198 | DNA | Microbulbifer |
| | | | agarilyticus |
| Phytase coding sequence (BPP) | 199 | DNA | Acinetobacter nectaris |
| Phytase coding sequence (BPP) | 200 | DNA | Catenovulum |
| | | | agarivorans |
| Phytase coding sequence (HAP) | 201 | DNA | Corynebacterium |
| | | | timonense |
| Phytase coding sequence (BPP) | 202 | DNA | Alteromonas macleodii |
| Phytase coding sequence (CPhy) | 203 | DNA | Clostridium sp. |
| Phytase coding sequence (CPhy) | 204 | DNA | Clostridium beijerinckii |
| Phytase coding sequence (CPhy) | 205 | DNA | Bdellovibrio |
| | | | bacteriovorus |
| Phytase coding sequence (CPhy) | 206 | DNA | Clostridium |
| | | | tyrobutyricum |
| Phytase coding sequence (CPhy) | 207 | DNA | Acidaminococcus |
| | | | fermentans |
| Phytase coding sequence (CPhy) | 208 | DNA | Desulfovibrio |
| | | | alcoholivorans |
| Phytase coding sequence (CPhy) | 209 | DNA | Desulfovibrio frigidus |
| Phytase coding sequence (CPhy) | 210 | DNA | Clostridium |
| | | | acetobutylicum |
| Phytase coding sequence (CPhy) | 211 | DNA | Allisonella |
| | | | histaminiformans |
| Phytase coding sequence (CPhy) | 212 | DNA | Centipeda periodontii |
| Phytase coding sequence (CPhy) | 213 | DNA | Desulfovibrio |
| | | | magneticus |
| Phytase coding sequence (HAP) | 214 | DNA | Burkholderiales |
| | | | bacterium |
| Phytase coding sequence (HAP) | 215 | DNA | Acinetobacter nectaris |
| Phytase coding sequence (HAP) | 216 | DNA | Dyella marensis |
| Phytase coding sequence (HAP) | 217 | DNA | Escherichia coli |
| Phytase coding sequence (HAP) | 218 | DNA | Anaerovibrio sp. |
| Phytase coding sequence (HAP) | 219 | DNA | Avibacterium |
| | | | para gallinarum |
| Phytase coding sequence (HAP) | 220 | DNA | Cronobacter sakazakii |
| Phytase coding sequence (HAP) | 221 | DNA | unidentified |
| Phytase coding sequence (HAP) | 222 | DNA | Corynebacterium |
| | | | singulare |
| Gluconate 2-dehydrogenase cytochrome c subunit | 223 | PRT | Pseudomonas putida |
| Gluconate 2-dehydrogenase flavoprotein subunit | 224 | PRT | Pseudomonas putida |
| Gluconate 2-dehydrogenase gamma subunit | 225 | PRT | Pseudomonas putida |
| Acid phosphatase AcpA | 226 | PRT | Burkholderia |
| | | | pseudomallei |
| Acid Phosphatase (Pho C) | 227 | PRT | Acetobacter pomorum |
| Acid Phosphatase Morganella morganii aphA | 228 | PRT | Morganella morganii |
| A. brasilense glutamine synthetase protein (GlnA) | 229 | PRT | Azospirillum brasilense |
| P. stutzeri glutamine synthetase protein (GlnA) | 230 | PRT | Pseudomonas stutzeri |
| A. brasilense GS adenylyltransferase protein (GlnE) | 231 | PRT | Azospirillum brasilense |
| A. caulinodans GS adenylyltransferase protein (GlnE) | 232 | PRT | Azorhizobium |
| | | | caulinodans |
| E. coli GS adenylyltransferase protein (GlnE) | 233 | PRT | Escherichia coli |
| E. coli PhoB box 1 | 234 | DNA | Escherichia coli |
| E. coli PhoB box 2 | 235 | DNA | Escherichia coli |
| E. coli PhoB box 3 | 236 | DNA | Escherichia coli |
| E. coli PhoB box 4 | 237 | DNA | Escherichia coli |
| E. coli PhoB box 5 | 238 | DNA | Escherichia coli |
| E. coli PhoB box 6 | 239 | DNA | Escherichia coli |
| E. coli PhoB box 7 | 240 | DNA | Escherichia coli |
| E. coli PhoB box 8 | 241 | DNA | Escherichia coli |
| E. coli PhoB box 9 | 242 | DNA | Escherichia coli |
| E. coli PhoB box 10 | 243 | DNA | Escherichia coli |
| E. coli PhoB box 11 | 244 | DNA | Escherichia coli |
| E. coli PhoB box 12 | 245 | DNA | Escherichia coli |
| E. coli PhoB box 13 | 246 | DNA | Escherichia coli |
| E. coli PhoB box 14 | 247 | DNA | Escherichia coli |
| E. coli PhoB box 15 | 248 | DNA | Escherichia coli |
| E. coli PhoB box 16 | 249 | DNA | Escherichia coli |
| E. coli PhoB box 17 | 250 | DNA | Escherichia coli |
| E. coli PhoB box 18 | 251 | DNA | Escherichia coli |
| E. coli PhoB box 19 | 252 | DNA | Escherichia coli |
| E. coli PhoB box 20 | 253 | DNA | Escherichia coli |
| E. coli PhoB box 21 | 254 | DNA | Escherichia coli |
| E. coli PhoB box 22 | 255 | DNA | Escherichia coli |
| E. coli PhoB box 23 | 256 | DNA | Escherichia coli |
| E. coli PhoB box 24 | 257 | DNA | Escherichia coli |
| E. coli PhoB box 25 | 258 | DNA | Escherichia coli |
| E. coli PhoB box 26 | 259 | DNA | Escherichia coli |
| E. coli PhoB box 27 | 260 | DNA | Escherichia coli |
| E. coli PhoB box 28 | 261 | DNA | Escherichia coli |
| E. coli PhoB box 29 | 262 | DNA | Escherichia coli |
| E. coli PhoB box 30 | 263 | DNA | Escherichia coli |
| E. coli PhoB box 31 | 264 | DNA | Escherichia coli |
| E. coli PhoB box 32 | 265 | DNA | Escherichia coli |
| E. coli PhoB box 33 | 266 | DNA | Escherichia coli |
| E. coli PhoB box 34 | 267 | DNA | Escherichia coli |
| E. coli PhoB box 35 | 268 | DNA | Escherichia coli |
| E. coli PhoB box 36 | 269 | DNA | Escherichia coli |
| E. coli PhoB box 37 | 270 | DNA | Escherichia coli |
| E. coli PhoB box 38 | 271 | DNA | Escherichia coli |
| E. coli PhoB box 39 | 272 | DNA | Escherichia coli |
| E. coli PhoB box 40 | 273 | DNA | Escherichia coli |
| E. coli PhoB box 41 | 274 | DNA | Escherichia coli |
| E. coli PhoB box 42 | 275 | DNA | Escherichia coli |
| E. coli PhoB box 43 | 276 | DNA | Escherichia coli |
| E. coli PhoB box 44 | 277 | DNA | Escherichia coli |
| E. coli PhoB box 45 | 278 | DNA | Escherichia coli |
| E. coli PhoB box 46 | 279 | DNA | Escherichia coli |
| E. coli PhoB box 47 | 280 | DNA | Escherichia coli |
| E. coli PhoB box 48 | 281 | DNA | Escherichia coli |
| E. coli PhoB box 49 | 282 | DNA | Escherichia coli |
| E. coli PhoB box 50 | 283 | DNA | Escherichia coli |
| E. coli PhoB box 51 | 284 | DNA | Escherichia coli |
| E. coli PhoB box 52 | 285 | DNA | Escherichia coli |
| E. coli PhoB box 53 | 286 | DNA | Escherichia coli |
| E. coli PhoB box 54 | 287 | DNA | Escherichia coli |
| E. coli PhoB box 55 | 288 | DNA | Escherichia coli |
| E. coli PhoB box 56 | 289 | DNA | Escherichia coli |
| E. coli PhoB box 57 | 290 | DNA | Escherichia coli |
| E. coli PhoB box 58 | 291 | DNA | Escherichia coli |
| E. coli PhoB box 59 | 292 | DNA | Escherichia coli |
| E. coli PhoB box 60 | 293 | DNA | Escherichia coli |
| E. coli PhoB box 61 | 294 | DNA | Escherichia coli |
| E. coli PhoB box 62 | 295 | DNA | Escherichia coli |
| E. coli PhoB box 63 | 296 | DNA | Escherichia coli |
| E. coli PhoB box 64 | 297 | DNA | Escherichia coli |
| E. coli PhoB box 65 | 298 | DNA | Escherichia coli |
| E. coli PhoB box 66 | 299 | DNA | Escherichia coli |
| E. coli PhoB box 67 | 300 | DNA | Escherichia coli |
| E. coli PhoB box 68 | 301 | DNA | Escherichia coli |
| E. coli PhoB box 69 | 302 | DNA | Escherichia coli |
| E. coli PhoB box 70 | 303 | DNA | Escherichia coli |
| E. coli PhoB box 71 | 304 | DNA | Escherichia coli |
| E. coli PhoB box 72 | 305 | DNA | Escherichia coli |
| E. coli PhoB box 73 | 306 | DNA | Escherichia coli |
| E. coli PhoB box 74 | 307 | DNA | Escherichia coli |
| E. coli PhoB box 75 | 308 | DNA | Escherichia coli |
| E. coli PhoB box 76 | 309 | DNA | Escherichia coli |
| E. coli PhoB box 77 | 310 | DNA | Escherichia coli |
| E. coli PhoB box 78 | 311 | DNA | Escherichia coli |
| E. coli PhoB box 79 | 312 | DNA | Escherichia coli |
| E. coli PhoB box 80 | 313 | DNA | Escherichia coli |
| E. coli PhoB box 81 | 314 | DNA | Escherichia coli |
| E. coli PhoB box 82 | 315 | DNA | Escherichia coli |
| E. coli PhoB box 83 | 316 | DNA | Escherichia coli |
| E. coli PhoB box 84 | 317 | DNA | Escherichia coli |
| E. coli PhoB box 85 | 318 | DNA | Escherichia coli |
| E. coli PhoB box 86 | 319 | DNA | Escherichia coli |
| E. coli PhoB box 87 | 320 | DNA | Escherichia coli |
| E. coli PhoB box 88 | 321 | DNA | Escherichia coli |
| E. coli PhoB box 89 | 322 | DNA | Escherichia coli |
| E. coli PhoB box 90 | 323 | DNA | Escherichia coli |
| E. coli PhoB box 91 | 324 | DNA | Escherichia coli |
| E. coli PhoB box 92 | 325 | DNA | Escherichia coli |
| E. coli PhoB box 93 | 326 | DNA | Escherichia coli |
| E. coli PhoB box 94 | 327 | DNA | Escherichia coli |
| E. coli PhoB box 95 | 328 | DNA | Escherichia coli |
| E. coli PhoB box 96 | 329 | DNA | Escherichia coli |
| E. coli PhoB box 97 | 330 | DNA | Escherichia coli |
| E. coli PhoB box 98 | 331 | DNA | Escherichia coli |
| E. coli PhoB box 99 | 332 | DNA | Escherichia coli |
| E. coli PhoB box 100 | 333 | DNA | Escherichia coli |
| E. coli PhoB box 101 | 334 | DNA | Escherichia coli |
| E. coli PhoB box 102 | 335 | DNA | Escherichia coli |
| E. coli PhoB box 103 | 336 | DNA | Escherichia coli |
| E. coli PhoB box 104 | 337 | DNA | Escherichia coli |
| E. coli PhoB box 105 | 338 | DNA | Escherichia coli |
| E. coli PhoB box 106 | 339 | DNA | Escherichia coli |
| E. coli PhoB box 107 | 340 | DNA | Escherichia coli |
| E. coli PhoB box 108 | 341 | DNA | Escherichia coli |
| E. coli PhoB box 109 | 342 | DNA | Escherichia coli |
| E. coli PhoB box 110 | 343 | DNA | Escherichia coli |
| E. coli PhoB box 111 | 344 | DNA | Escherichia coli |
| E. coli PhoB box 112 | 345 | DNA | Escherichia coli |
| S. coelicolor PhoP box 1 | 346 | DNA | Streptomyces coelicolor |
| S. coelicolor PhoP box 2 | 347 | DNA | Streptomyces coelicolor |
| S. coelicolor PhoP box 3 | 348 | DNA | Streptomyces coelicolor |
| S. coelicolor PhoP box 4 | 349 | DNA | Streptomyces coelicolor |
| S. coelicolor PhoP box 5 | 350 | DNA | Streptomyces coelicolor |
| S. coelicolor PhoP box 6 | 351 | DNA | Streptomyces coelicolor |
| S. coelicolor PhoP box 7 | 352 | DNA | Streptomyces coelicolor |
| S. coelicolor PhoP box 8 | 353 | DNA | Streptomyces coelicolor |
| S. coelicolor PhoP box 9 | 354 | DNA | Streptomyces coelicolor |
| S. coelicolor PhoP box 10 | 355 | DNA | Streptomyces coelicolor |
| S. coelicolor PhoP box 11 | 356 | DNA | Streptomyces coelicolor |
| S. coelicolor PhoP box 12 | 357 | DNA | Streptomyces coelicolor |
| S. coelicolor PhoP box 13 | 358 | DNA | Streptomyces coelicolor |
| S. coelicolor PhoP box 14 | 359 | DNA | Streptomyces coelicolor |
| S. coelicolor PhoP box 15 | 360 | DNA | Streptomyces coelicolor |
| S. coelicolor PhoP box 16 | 361 | DNA | Streptomyces coelicolor |
| S. coelicolor PhoP box 17 | 362 | DNA | Streptomyces coelicolor |
| S. coelicolor PhoP box 18 | 363 | DNA | Streptomyces coelicolor |
| S. coelicolor PhoP box 19 | 364 | DNA | Streptomyces coelicolor |
| S. coelicolor PhoP box 20 | 365 | DNA | Streptomyces coelicolor |
| S. coelicolor PhoP box 21 | 366 | DNA | Streptomyces coelicolor |
| S. coelicolor PhoP box 22 | 367 | DNA | Streptomyces coelicolor |
| S. coelicolor PhoP box 23 | 368 | DNA | Streptomyces coelicolor |
| S. coelicolor PhoP box 24 | 369 | DNA | Streptomyces coelicolor |
| S. coelicolor PhoP box 25 | 370 | DNA | Streptomyces coelicolor |
| S. coelicolor PhoP box 26 | 371 | DNA | Streptomyces coelicolor |
| S. coelicolor PhoP box 27 | 372 | DNA | Streptomyces coelicolor |
| S. coelicolor PhoP box 28 | 373 | DNA | Streptomyces coelicolor |
| S. coelicolor PhoP box 29 | 374 | DNA | Streptomyces coelicolor |
| S. coelicolor PhoP box 30 | 375 | DNA | Streptomyces coelicolor |
| A. caulinodans PstS box 1 | 376 | DNA | Azorhizobium |
| | | | caulinodans |
| A. brasilense PstS box 1 | 377 | DNA | Azospirillum brasilense |
| E. coli PhoA box 1 | 378 | DNA | Escherichia coli |
| E. coli PstS box 1 | 379 | DNA | Escherichia coli |
| E. coli PstS box 2 | 380 | DNA | Escherichia coli |
| E. coli PstS box 3 | 381 | DNA | Escherichia coli |
| E. coli PstS box 4 | 382 | DNA | Escherichia coli |
| G. diazotrophicus PstS box 1 | 383 | DNA | Gluconacetobacter |
| | | | diazotrophicus |
| G. diazotrophicus PstS box 2 | 384 | DNA | Gluconacetobacter |
| | | | diazotrophicus |
| H. seropedicae PstS box 1 | 385 | DNA | Herbaspirillum |
| | | | seropedicae |
| K. radicincitans PstS box 1 | 386 | DNA | Kosakonia radicincitans |
| K. radicincitans PstS box 2 | 387 | DNA | Kosakonia radicincitans |
| K. radicincitans PstS box 3 | 388 | DNA | Kosakonia radicincitans |
| K. radicincitans PstS box 4 | 389 | DNA | Kosakonia radicincitans |
| K. radicincitans PstS box 5 | 390 | DNA | Kosakonia radicincitans |
| P. azotofixans PstS box 1 | 391 | DNA | Paenibacillus azotofixans |
| P. stutzeri PhoX box 1 | 392 | DNA | Pseudomonas stutzeri |
| P. stutzeri PhoX box 2 | 393 | DNA | Pseudomonas stutzeri |
| P. stutzeri PhoX box 3 | 394 | DNA | Pseudomonas stutzeri |
| P. stutzeri PhoX box 4 | 395 | DNA | Pseudomonas stutzeri |
| P. stutzeri Phy box 1 | 396 | DNA | Pseudomonas stutzeri |
| P. stutzeri PstS box 1 | 397 | DNA | Pseudomonas stutzeri |
| K. radicincitans PhoA alkaline phosphatase | 398 | PRT | Kosakonia radicincitans |
| K. radicincitans PstS phosphate transporter ABC | 399 | PRT | Kosakonia radicincitans |
| P. durus PhoA alkaline phosphatase | 400 | PRT | Paenibacillus durus |
| P. durus PstS phosphate ABC transporter | 401 | PRT | Paenibacillus durus |
| A. caulinodans PhoA alkaline phosphatase | 402 | PRT | Azorhizobium |
| | | | caulinodans |
| A. caulinodans PstS phosphate ABC transporter | 403 | PRT | Azorhizobium |
| | | | caulinodans |
| E. coli PhoA alkaline phosphatase | 404 | PRT | Escherichia coli |
| E. coli PstS phosphate ABC transporter | 405 | PRT | Escherichia coli |
| A. olearius PhoA alkaline phosphatase | 406 | PRT | Azoarcus olearius |
| A. olearius PstS phosphate ABC transporter | 407 | PRT | Azoarcus olearius |
| S. stutzeri PhoX family phosphatase | 408 | PRT | Stutzerimonas stutzeri |
| S. stutzeri PstS phosphate ABC transporter | 409 | PRT | Stutzerimonas stutzeri |
| P. aeruginosa PhoX family phosphatase | 410 | PRT | Pseudomonas aeruginosa |
| A. aromaticum PhoX family phosphatase | 411 | PRT | Aromatoleum |
| | | | aromaticum |
| Y. vestfoldensis PhoX family phosphatase | 412 | PRT | Yoonia vestfoldensis |
| M. alkaliphilus PhoX family phosphatase | 413 | PRT | Maritimibacter |
| | | | alkaliphilus |
| B. subtilis phytase | 414 | PRT | Bacillus subtilis |
| H. seropedicae PstS phosphate ABC transporter | 415 | PRT | Herbaspirillum |
| | | | seropedicae |
| G. diazotrophicus PstS phosphate ABC transporter | 416 | PRT | Gluconacetobacter |
| | | | diazotrophicus |
| A. caulinodans pstS promoter | 417 | DNA | Azorhizobium |
| | | | caulinodans |
| A. brasilense pstS promoter | 418 | DNA | Azospirillum brasilense |
| E. coli phoA promoter | 419 | DNA | Escherichia coli |
| E. coli pstS promoter | 420 | DNA | Escherichia coli |
| G. diazotrophicus pstS promoter | 421 | DNA | Gluconacetobacter |
| | | | diazotrophicus |
| H. seropedicae pstS promoter | 422 | DNA | Herbaspirillum |
| | | | seropedicae |
| K. radicincitans pstS promoter 1 | 423 | DNA | Kosakonia radicincitans |
| K. radicincitans pstS promoter 2 | 424 | DNA | Kosakonia radicincitans |
| P. azotofixans pstS promoter | 425 | DNA | Paenibacillus azotofixans |
| P. stutzeri phoX promoter | 426 | DNA | Pseudomonas stutzeri |
| P. stutzeri phy promoter | 427 | DNA | Pseudomonas stutzeri |
| P. stutzeri pstS promoter | 428 | DNA | Pseudomonas stutzeri |
| BCD 1 ribosome binding site | 429 | DNA | synthetic construct |
| BCD 2 vL ribosome binding site | 430 | DNA | synthetic construct |
| BCD 2 vS ribosome binding site | 431 | DNA | synthetic construct |
| BCD 5 ribosome binding site | 432 | DNA | synthetic construct |
| BCD 6 ribosome binding site | 433 | DNA | synthetic construct |
| BCD 7 ribosome binding site | 434 | DNA | synthetic construct |
| BCD 8 ribosome binding site | 435 | DNA | synthetic construct |
| BCD 9 ribosome binding site | 436 | DNA | synthetic construct |
| BCD 10 ribosome binding site | 437 | DNA | synthetic construct |
| BCD 11 ribosome binding site | 438 | DNA | synthetic construct |
| BCD 12 ribosome binding site | 439 | DNA | synthetic construct |
| BCD 13 ribosome binding site | 440 | DNA | synthetic construct |
| BCD 14 ribosome binding site | 441 | DNA | synthetic construct |
| BCD 15 ribosome binding site | 442 | DNA | synthetic construct |
| BCD 16 ribosome binding site | 443 | DNA | synthetic construct |
| BCD 17 ribosome binding site | 444 | DNA | synthetic construct |
| BCD 18 ribosome binding site | 445 | DNA | synthetic construct |
| BCD 19 ribosome binding site | 446 | DNA | synthetic construct |
| BCD 20 ribosome binding site | 447 | DNA | synthetic construct |
| BCD 21 ribosome binding site | 448 | DNA | synthetic construct |
| BCD 22 vL ribosome binding site | 449 | DNA | synthetic construct |
| BCD 22 vS ribosome binding site | 450 | DNA | synthetic construct |
| BCD 23 ribosome binding site | 451 | DNA | synthetic construct |
| BCD 24 ribosome binding site | 452 | DNA | synthetic construct |
| BBa B0029 ribosome binding site | 453 | DNA | synthetic construct |
| BBa B0030 ribosome binding site | 454 | DNA | synthetic construct |
| BBa B0034 ribosome binding site | 455 | DNA | synthetic construct |
| BBa_B1002 terminator | 456 | DNA | synthetic construct |
| BBa_B1006 terminator | 457 | DNA | synthetic construct |
| BBa_B1015 terminator | 458 | DNA | synthetic construct |
| Pliar53 promoter | 459 | DNA | synthetic construct |
| Acetobacter malorum GlnA protein sequence | 460 | PRT | Acetobacter malorum |
| Acetobacter tropicalis GlnA protein sequence | 461 | PRT | Acetobacter tropicalis |
| Acetobacter pasteurianus GlnA protein sequence | 462 | PRT | Acetobacter pasteurianus |
| Acidothermus cellulolyticus GlnA protein sequence | 463 | PRT | Acidothermus |
| | | | cellulolyticus |
| Acinetobacter baumannii GlnA protein sequence | 464 | PRT | Acinetobacter baumannii |
| Acinetobacter calcoaceticus GlnA protein sequence | 465 | PRT | Acinetobacter |
| | | | calcoaceticus |
| Agrobacterium deltaense GlnA protein sequence | 466 | PRT | Agrobacterium deltaense |
| Aromatoleum aromaticum GlnA protein sequence | 467 | PRT | Aromatoleum |
| | | | aromaticum |
| Arthrobacer sp. GlnA protein sequence | 468 | PRT | Arthrobacer sp. |
| Azoarcus sp. GlnA protein sequence | 469 | PRT | Azoarcus sp. |
| Azorhizobium caulinodans GlnA protein sequence | 470 | PRT | Azorhizobium |
| | | | caulinodans |
| Azotobacter vinelandii GlnA protein sequence | 471 | PRT | Azotobacter vinelandii |
| Bacillus subtilis GlnA protein sequence | 472 | PRT | Bacillus subtilis |
| Bifidobacterium adolescentis GlnA protein sequence | 473 | PRT | Bifidobacterium |
| | | | adolescentis |
| Bifidobacterium asteroides GlnA protein sequence | 474 | PRT | Bifidobacterium |
| | | | asteroides |
| Bradyrhizobium diazoefficiens GlnA protein sequence | 475 | PRT | Bradyrhizobium |
| | | | diazoefficiens |
| Bradyrhizobium frederickii GlnA protein sequence | 476 | PRT | Bradyrhizobium |
| | | | frederickii |
| Burkholderia cenocepacia GlnA protein sequence | 477 | PRT | Burkholderia |
| | | | cenocepacia |
| Burkholderia stagnalis GlnA protein sequence | 478 | PRT | Burkholderia stagnalis |
| Conexibacter sp. GlnA protein sequence | 479 | PRT | Conexibacter sp. |
| Curtobacterium albidum GlnA protein sequence | 480 | PRT | Curtobacterium albidum |
| Ensifer adhaerens GlnA protein sequence | 481 | PRT | Ensifer adhaerens |
| Enterobacter agglomerans GlnA protein sequence | 482 | PRT | Enterobacter |
| | | | agglomerans |
| Erwinia amylovora GlnA protein sequence | 483 | PRT | Erwinia amylovora |
| Escherichia coli GlnA protein sequence | 484 | PRT | Escherichia coli |
| Flavobacterium glycines GlnA protein sequence | 485 | PRT | Flavobacterium glycines |
| Frankia canadensis GlnA protein sequence | 486 | PRT | Frankia canadensis |
| Gluconacetobacter diazotrophicus GlnA protein | 487 | PRT | Gluconacetobacter |
| sequence | | | diazotrophicus |
| Gluconobacter oxydans GlnA protein sequence | 488 | PRT | Gluconobacter oxydans |
| Herbaspirillum seropedicae GlnA protein sequence | 489 | PRT | Herbaspirillum |
| | | | seropedicae |
| Klebsiella oxytoca GlnA protein sequence | 490 | PRT | Klebsiella oxytoca |
| Klebsiella variicola GlnA protein sequence | 491 | PRT | Klebsiella variicola |
| Kosakonia sp. GlnA protein sequence | 492 | PRT | Kosakonia sp. |
| Lactobacillus helveticus GlnA protein sequence | 493 | PRT | Lactobacillus helveticus |
| Lactococcus laudensis GlnA protein sequence | 494 | PRT | Lactococcus laudensis |
| Lysinibacillus halotolerans GlnA protein sequence | 495 | PRT | Lysinibacillus |
| | | | halotolerans |
| Maritimibacter harenae GlnA protein sequence | 496 | PRT | Maritimibacter harenae |
| Methylobacterium aquaticum GlnA protein sequence | 497 | PRT | Methylobacterium |
| | | | aquaticum |
| Paenibacillus polymyxa GlnA protein sequence | 498 | PRT | Paenibacillus polymyxa |
| Priestia megaterium GlnA protein sequence | 499 | PRT | Priestia megaterium |
| Pseudarthrobacter siccitolerans GlnA protein sequence | 500 | PRT | Pseudarthrobacter |
| | | | siccitolerans |
| Pseudomonas azotoformans GlnA protein sequence | 501 | PRT | Pseudomonas |
| | | | azotoformans |
| Pseudomonas putida GlnA protein sequence | 502 | PRT | Pseudomonas putida |
| Pseudomonas syringae GlnA protein sequence | 503 | PRT | Pseudomonas syringae |
| Rahnella contaminans GlnA protein sequence | 504 | PRT | Rahnella contaminans |
| Rahnella sp. GlnA protein sequence | 505 | PRT | Rahnella sp. |
| Rhizobium leguminosarum GlnA protein sequence | 506 | PRT | Rhizobium |
| | | | leguminosarum |
| Rhodococcus oryzae GlnA protein sequence | 507 | PRT | Rhodococcus oryzae |
| Sphingomonas glacialis GlnA protein sequence | 508 | PRT | Sphingomonas glacialis |
| Stenotrophomonas maltophilia GlnA protein sequence | 509 | PRT | Stenotrophomonas |
| | | | maltophilia |
| Stutzerimonas stutzeri GlnA protein sequence | 510 | PRT | Stutzerimonas stutzeri |
| Variovorax paradoxus GlnA protein sequence | 511 | PRT | Variovorax paradoxus |
| Yoonia vestfoldensis GlnA protein sequence | 512 | PRT | Yoonia vestfoldensis |
| R. aceris PstS phosphate ABC transporter | 513 | PRT | Rahnella aceris |
| PstS phosphate ABC transporter consensus motif | 514 | PRT | synthetic construct |
| (GSTVNWP[T/V\|GLGGKGNDG\|V/I]AAFVQR | | | |
| LPGSIGYVEYAYAKQNNL[A/T][Y/W]TKL | | | |
| [V/F][D/S]ADGK; SEQ ID NO: 514) | | | |
| K. variicola pstS promoter | 515 | DNA | Klebsiella variicola |
| Optimized TetR | 516 | PRT | synthetic construct |
| K. sacchari pstS promoter | 517 | DNA | Kosakonia sacchari |
| R. aceris pstS promoter | 518 | DNA | Rahnella aceris |
| K. sacchari glutamine synthetase | 519 | PRT | Kosakonia sacchari |
| K. variicola glutamine synthetase | 520 | PRT | Klebsiella variicola |
| R. aceris glutamine synthetase | 521 | PRT | Rahnella aceris |
| Control GFP reporter gene expression cassette | 522 | DNA | synthetic construct |
| K. variicola PstS phosphate transporter ABC | 523 | PRT | Klebsiella variicola |
| K. sacchari PstS phosphate transporter ABC | 524 | PRT | Kosakonia sacchari |
| K. variicola pstS promoter PHO BOX 1 | 525 | DNA | Klebsiella variicola |
| K. variicola pstS promoter PHO BOX 2 | 526 | DNA | Klebsiella variicola |
| K. variicola pstS promoter PHO BOX 3 | 527 | DNA | Klebsiella variicola |
| K. variicola pstS promoter PHO BOX 4 | 528 | DNA | Klebsiella variicola |
| K. variicola pstS promoter PHO BOX 5 | 529 | DNA | Klebsiella variicola |
| P. graminis glutamine synthetase | 530 | PRT | Paenibacillus graminis |
| Enterobacter sp. glutamine synthetase | 531 | PRT | Enterobacter sp |
| P. diazotrophicus glutamine synthetase | 532 | PRT | Phytobacter |
| | | | diazotrophicus |
| E. dykesii glutamine synthetase | 533 | PRT | Enterobacter dykesii |

TABLE 6
Non-limiting examples of protease-specific
recognition sequence (PSRS) insertion
sites in glnA and glnE target genes for ammonia
release. Corresponding insertion sites in
other glnA and glnE genes including those set forth in
Table 3, Table 5, and the sequence listing are also provided.

| | | | Name of | | | | |
|---|---|---|---|---|---|---|---|
| | Target | SEQ | Cut Site for | N- | N- | C- | C- |
| | Gene | ID | insertion of | terminal | terminal | terminal | terminal |
| Strain | Name | NO | PSRS | AA | AA # | AA | AA # |
| A. brasilense | glnA | 229 | 1 | T | 98 | G | 99 |
| Sp245 | | | | | | | |
| A. brasilense | glnA | 229 | 2 | G | 121 | I | 122 |
| Sp245 | | | | | | | |
| A. brasilense | glnA | 229 | 3 | G | 279 | Q | 280 |
| Sp245 | | | | | | | |
| A. brasilense | glnA | 229 | 4 | G | 285 | N | 286 |
| Sp245 | | | | | | | |
| P. stutzeri | glnA | 230 | 1 | Q | 98 | G | 99 |
| DSM 4166 | | | | | | | |
| P. stutzeri | glnA | 230 | 2 | G | 119 | I | 120 |
| DSM 4166 | | | | | | | |
| P. stutzeri | glnA | 230 | 3 | A | 283 | G | 284 |
| DSM 4166 | | | | | | | |
| P. stutzeri | glnA | 230 | 4 | I | 298 | G | 299 |
| DSM 4166 | | | | | | | |
| A. brasilense | glnE | 231 | 1 | Q | 448 | L | 449 |
| Sp245 | | | | | | | |
| A. brasilense | glnE | 231 | 2 | D | 480 | P | 481 |
| Sp245 | | | | | | | |
| A. brasilense | glnE | 231 | 3 | W | 505 | H | 506 |
| Sp245 | | | | | | | |
| A. brasilense | glnE | 231 | 4 | A | 528 | M | 529 |
| Sp245 | | | | | | | |
| A. brasilense | glnE | 231 | 5 | H | 624 | N | 625 |
| Sp245 | | | | | | | |
| A. | glnE | 232 | 1 | P | 527 | R | 528 |
| caulinodans | | | | | | | |
| ORS 571 | | | | | | | |
| A. | glnE | 232 | 2 | E | 537 | H | 538 |
| caulinodans | | | | | | | |
| ORS 571 | | | | | | | |
| A. | glnE | 232 | 3 | I | 547 | E | 548 |
| caulinodans | | | | | | | |
| ORS 571 | | | | | | | |
| E. coli | glnE | 233 | 1 | T | 422 | A | 423 |
| E. coli | glnE | 233 | 2 | D | 608 | D | 609 |

### EXAMPLES

### Example 1. Identification and Selection of Phosphate-Sensitive Promoters and Pho Boxes

The phosphate (Pho) regulon is a bacterial regulatory mechanism that senses and responds to changing availability and concentration of inorganic phosphate (Pi). The Pho regulon activates the expression of extracellular enzymes, phosphate-specific carriers, and enzymes involved in phosphate storage and preservation. The most conserved member of the Pho regulon in bacteria is the high affinity Pi transporter (Pst), and the most common enzymes induced in response to inorganic phosphate starvation in bacteria are alkaline phosphatases (PhoA, PhoX), phospholipases (PhoD), glycerophosphodiester phosphodiesterases, phytases (Phy), 5′-nucleotidase, and the Pst itself.

The Pho regulon is controlled by a two-component regulatory system that includes an inner membrane histidine kinase sensor protein and a regulator of the cytoplasmic transcriptional response. These proteins are named differently in some bacteria, for example, PhoR-PhoB in Escherichia coli, PhoR-PhoP in Bacillus subtilis, PnpR-PnpS in Streptococcus pneumoniae. In all cases of inorganic phosphate deficiency, the response regulator is phosphorylated at an aspartic acid residue by the sensor kinase. The phosphorylated response regulator binds to specific DNA sequences and activates or inhibits gene transcription.

These specific regulator-binding DNA sequences that facilitate the activation or inhibition of gene expression in a phosphate-sensitive manner are termed Pho boxes. Pho boxes have been identified using ChIP-Seq, sequence similarity, and RNA-Seq in Escherichia coli and Streptomyces coelicolor (Allenby et al. (doi: 10.1093/nar/gks766) and Fitzgerald et al. (doi: 10.1128/mbio.02535-22), respectively; SEQ ID NO: 234-375).

Beyond the two-component PhoR-PhoB system, phosphate-sensitive bacterial responses involve additional proteins. By way of example, the E. coli inorganic phosphate uptake pathway requires five more proteins, four of them being the components of the Pst and one a component of the PhoU metal-binding protein.

Depleting inorganic phosphate in a growth medium activates the Pho regulon in bacteria in the medium. When the concentration of inorganic phosphate decreases, PhoB is activated by PhoR acting as a kinase, but under conditions of excess free phosphate, PhoB activation is interrupted by PhoR acting as a phosphatase. PhoU is required for PhoB dephosphorylation under phosphate-rich conditions. Although PhoU is found in many bacterial genomes, this gene is absent in B. subtilis. The inorganic phosphate-signaling network in this bacterium includes a positive feedback loop between the PhoP-PhoR and ResD-ResE two-component systems. ResD does not bind to the phoPR operon and appears to transfer its control through the expression of terminal oxidases.

To express an RNA sequence or protein of interest in a phosphate-sensitive manner in agriculturally relevant bacteria, promoters were constructed that are activated by decreasing phosphate concentration. Initially, sequence alignments were calculated for published Pho boxes to identify a consensus sequence so that additional phosphate-sensitive promoters could be identified simply by searching for sequences similar to the consensus sequence in agriculturally relevant bacteria. However, poor homology among published Pho boxes in bacteria of the same species made this approach unsuccessful. As a result, two distinct approaches were pursued to determine lists of Pho boxes that could be searched to find phosphate-sensitive promoters in agriculturally relevant bacteria.

In the first approach adapted from Torres-Bacete et al. (doi: 10.1111/1751-7915.13808), a DNA motif WebLogo and position-by-position DNA base probability were calculated from known Pho boxes. Based on these statistics, degenerate primers were synthesized to create a library of alternative Pho boxes via polymerase chain reaction (PCR) amplification of a DNA template comprising the sequences based on the aforementioned WebLogo. To construct a library of phosphate-sensitive reporter circuits, the DNA library of alternative Pho boxes above was integrated into the −35 region of a constitutive promoter (BG42), which was operably linked to a strong ribosome binding site (BCD2) and a green fluorescent protein (GFP) gene. The library of phosphate-sensitive GFP reporter circuits was then screened in Pseudomonas and Escherichia strains at varying phosphate concentrations to determine the Pho box and phosphate-sensitive promoter with the greatest response to changes in extracellular phosphate concentration. The Pho box from the maximally responding promoter was subsequently searched using the US National Center for Biotechnology Information (NCBI) Basic Local Alignment Search Tool (BLAST) to identify naturally occurring Pho boxes with high sequence similarity to the Pho box of the maximally responding phosphate-sensitive promoter identified above. Those promoters were screened for ability to induce phosphate-sensitive changes in gene expression as in EXAMPLE 2.

In the second approach, phosphate-sensitive promoter sequences were bioinformatically extracted from agriculturally relevant bacteria. Bacterial species across a range of agriculturally relevant and model taxa were selected, similar to those described by Santos-Beneit (doi: 10.3389/fmicb.2015.00402), Grillo-Puertas et al. (doi: 10.3389/fmicb.2021.666277), and Torres-Bacete et al. (doi: 10.1111/1751-7915.13808). Taxa searched for phosphate-sensitive promoters included both gram-positive bacteria such as Bacillus subtilis, Paenibacillus azotofixans, and Paenibacillus durus as well as gram-negative bacteria. Among gram-negative bacteria, several classes of soil bacteria were searched for phosphate-sensitive promoters, including: alphaproteobacteria like Azospirillum brasilense, Azorhizobium caulinodans, Gluconacetobacter diazotrophicus, Maritimibacter alkaliphilus, and Yoonia vestfoldensis; betaproteobacteria like Aromatoleum aromaticum, Azoarcus olearius, and Herbaspirillum seropedicae; and gammaproteobacteria like Escherichia coli, Kosakonia radicincitans, Pseudomonas aeruginosa, and Pseudomonas stutzeri. Genes of each bacterial Pho regulon were identified because these genes are known to have phosphate-sensitive expression. The presence of Pho boxes in each of the identified Pho regulon genes was confirmed by sequence similarity to Pho boxes described in Allenby et al. (doi: 10.1093/nar/gks766). Pho boxes present in agriculturally relevant bacteria are included as SEQ ID NO: 376-397. CLUSTAL alignment performed using Mview with default parameters showed that no consensus sequence could be easily constructed for these Pho boxes.

Since no consensus sequence emerged using the second approach, phosphate-sensitive promoters were identified by determining the DNA regions immediately upstream of the protein-coding regions of Pho regulon genes in the agriculturally relevant bacterial taxa. The protein-coding sequences of certain Pho regulon genes (given by SEQ ID NO: 398-416, TABLE 7) were determined by querying the US National Center for Biotechnology Information (NCBI) Protein database. The sequence similarity among homologous Pho regulon genes was determined by CLUSTAL alignment using the M-Coffee multiple sequence aligner with default parameters, and multiple sequence alignment statistics were calculated using Mview (SEQ ID NO: 398-416, TABLE 7). Subsequently, promoter DNA sequences were extracted by locating regions immediately upstream of the start codon of the phoA, phoX, phy, and pstS genes associated with SEQ ID NO: 398-416, TABLE 7. DNA regions selected as promoters can comprise about 100 bp upstream of a relevant start codon but at least 50 bp upstream of the start codon and frequently about 50 to about 250 bp upstream of the relevant start codon (SEQ ID NO: 417-428, TABLE 8).

Subsequently, phosphate-sensitive promoter sequences were identified in agricultural soil isolates of Klebsiella variicola, Kosakonia sacchari, and Rahnella aceris using a method adapted from the method above. The phosphate ABC transporter substrate-binding protein PstS amino acid sequences of these soil isolates each has more than 76% homology to the Rahnella aceris PstS amino acid sequence (SEQ ID NO: 513), as well as a consensus motif shown in SEQ ID NO: 514. Additionally, the protein-coding sequences of the pstS genes in these Klebsiella variicola and Kosakonia sacchari isolates were determined to be SEQ ID NO: 523 and 524 respectively, with promoter DNA sequences for the three species given by SEQ ID NO: 515, 517, and 518.

Rows are organized by different bacterial species or strains. Columns are organized by different Pho regulon proteins. Cells contain the SEQ ID NO of each protein's amino acid sequence. “Absent” indicates that the bacterial species or strain does not have a gene encoding a specific Pho regulon protein. “Not tested” indicates that a specific species was not searched for a gene encoding a specific Pho regulon protein.

TABLE 7
Protein-coding sequences of Pho regulon genes
in agriculturally relevant and model bacteria.

| Bacterial Species/ | | | | |
|---|---|---|---|---|
| Strain | PhoA | PhoX | Phytase* | PstS |
| Kosakonia radicincitans | SEQ ID NO: 398 | Absent | Absent | SEQ ID NO: |
| DSM 16656 | | | | 399 |
| Paenibacillus durus | SEQ ID NO: 400 | Absent | Absent | SEQ ID NO: |
| ATCC 35681 | | | | 401 |
| Azorhizobium | SEQ ID NO: 402 | Absent | Absent | SEQ ID NO: |
| caulinodans ORS 571 | | | | 403 |
| Escherichia coli K12- | SEQ ID NO: 404 | Absent | Absent | SEQ ID NO: |
| MG1655 | | | | 405 |
| Azoarcus olearius | SEQ ID NO: 406 | Absent | Absent | SEQ ID NO: |
| DQS4 | | | | 407 |
| Stutzerimonas stutzeri | Absent | SEQ ID NO: 408 | Absent | SEQ ID NO: |
| CMT.A.9 | | | | 409 |
| Pseudomonas | Not tested | SEQ ID NO: 410 | Not tested | Not tested |
| aeruginosa | | | | |
| Aromatoleum | Not tested | SEQ ID NO: 411 | Not tested | Not tested |
| aromaticum | | | | |
| Yoonia vestfoldensis | Not tested | SEQ ID NO: 412 | Not tested | Not tested |
| Maritimibacter | Not tested | SEQ ID NO: 413 | Not tested | Not tested |
| alkaliphilus | | | | |
| Bacillus subtilis | Not tested | Not tested | SEQ ID NO: | Not tested |
| | | | 414 | |
| Herbaspirillum | Absent | Absent | Absent | SEQ ID NO: |
| seropedicae Z67 | | | | 415 |
| Gluconacetobacter | Absent | Absent | Absent | SEQ ID NO: |
| diazotrophicus PA1 5 | | | | 416 |

TABLE 8
DNA regions upstream of Pho regulon coding sequences identified as phosphate-sensitive promoters.
Rows are organized by different bacterial species or strains. Columns are organized by Pho regulon genes.
Cells contain promoter SEQ ID NO and any relevant Pho box SEQ ID NO. “Not tested” indicates
that a specific species was not searched for a specific Pho regulon gene promoter.

| Bacterial | | | | |
|---|---|---|---|---|
| Species/Strain | phoA | phoX | phy | pstS |
| Azorhizobium | Not tested | Not tested | Not tested | Promoter: SEQ ID NO 417 |
| caulinodans | | | | Pho boxes: SEQ ID NO 376 |
| Azospirillum | Not tested | Not tested | Not tested | Promoter: SEQ ID NO 418 |
| brasilense | | | | Pho boxes: SEQ ID NO 377 |
| Escherichia coli | Promoter: SEQ | Not tested | Not tested | Promoter: SEQ ID NO 420 |
| | ID NO 419 | | | Pho boxes: SEQ ID NO |
| | Pho boxes: SEQ | | | 379-382 |
| | ID NO 378 | | | |
| Glucon- | Not tested | Not tested | Not tested | Promoter: SEQ ID NO 421 |
| acetobacter | | | | Pho boxes: SEQ ID NO 383- |
| diazotrophicus | | | | 384 |
| Herbaspirillum | Not tested | Not tested | Not tested | Promoter: SEQ ID NO 422 |
| seropedicae | | | | Pho boxes: SEQ ID NO 385 |
| Kosakonia | Not tested | Not tested | Not tested | Promoters: SEQ ID NO 423- |
| radicincitans | | | | 424 |
| | | | | Pho boxes: SEQ ID NO 386- |
| | | | | 390 |
| Paenibacillus | Not tested | Not tested | Not tested | Promoter: SEQ ID NO 425 |
| azotofixans | | | | Pho boxes: SEQ ID NO 391 |
| Pseudomonas | Not tested | Promoter: SEQ | Promoter: SEQ | Promoter: SEQ ID NO 428 |
| stutzeri | | ID NO 426 | ID NO 427 | Pho boxes: SEQ ID NO 397 |
| | | Pho boxes: SEQ | Pho boxes: SEQ | |
| | | ID NO 392-395 | ID NO 396 | |

### Example 2. Engineering Bacteria to Express a Reporter Gene in Response to Decreasing Phosphate Concentration

To generate strains that express a GFP reporter gene in response to phosphate depletion, the pstS promoter regions of Azospirillum brasilense Sp245 (SEQ ID NO: 418), Herbaspirillum seropedicae Z67 (SEQ ID NO: 422), Pseudomonas stutzeri DSM 4166 (SEQ ID NO: 428), and Kosakonia radicincitans DSM16656 (SEQ ID NO: 423-424) were PCR amplified from genomic DNA, and the synthetic promoter Pliar53 (SEQ ID NO: 459) was synthesized, each with HiFi adapters for cloning into an entry plasmid digested with NotI. The cloning site was designed such that the Pi-dependent promoters were positioned upstream of GFP. Plasmids were confirmed by next generation sequencing and conjugated with aid of the helper strain pRK2013 from the E. coli DH5a donor strains into the strains listed above. Plasmid recipients were confirmed by antibiotic selection and by presence of constitutive LSSmScarlett fluorescence which was additionally conveyed by the cloning plasmid.

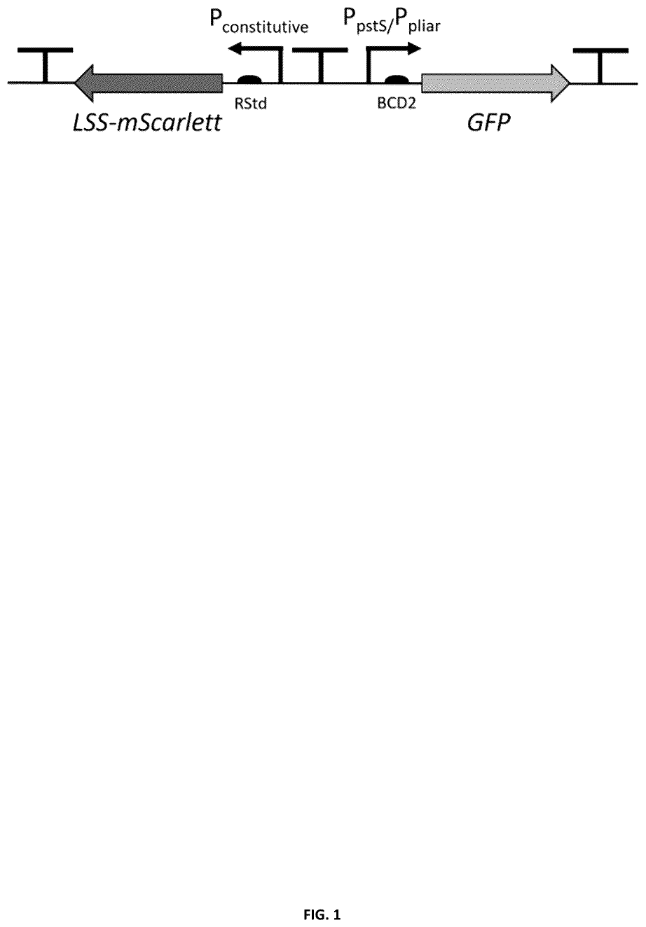

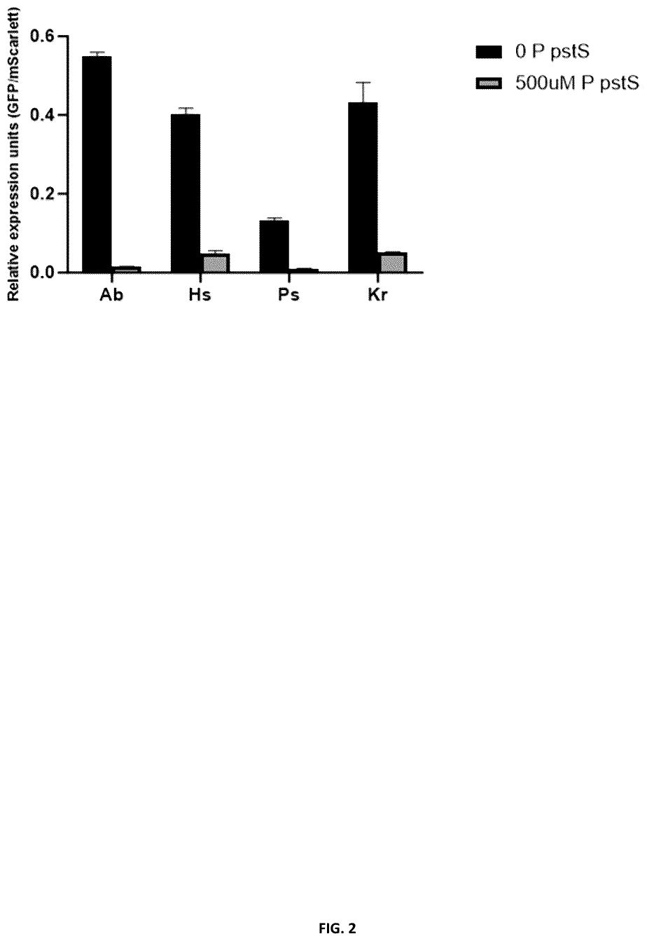

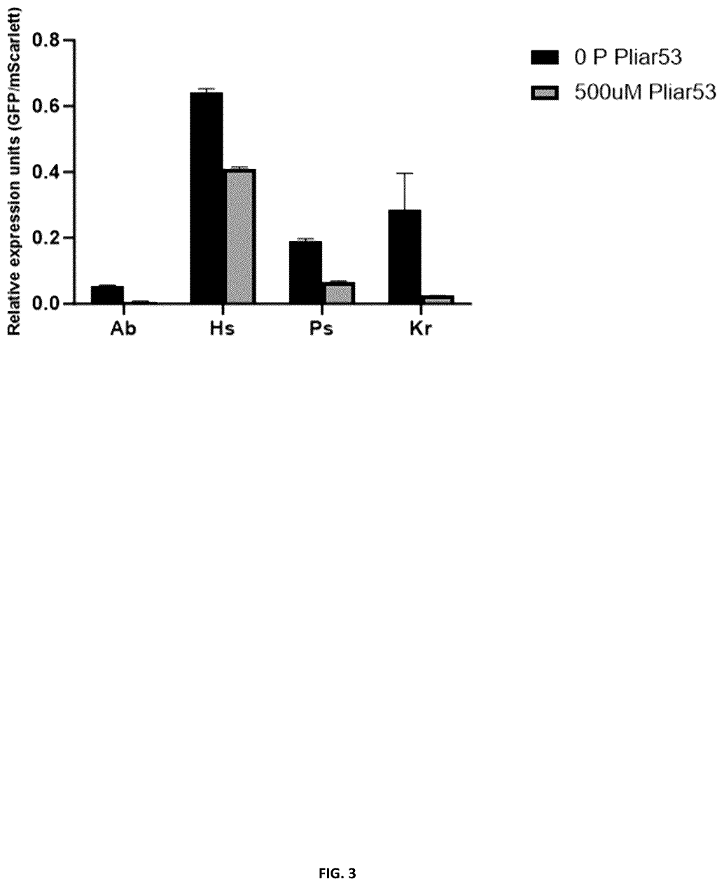

To confirm that pstS and Pliar53 promoters carried by Azospirillum brasilense Sp245, Herbaspirillum seropedicae Z67, Pseudomonas stutzeri DSM 4166, and Kosakonia radicincitans DSM16656 were specifically activated under Pi depleted conditions, expression from these promoters was measured in cultures (n=3) incubated for 15 hours in nitrogen-free basal high phosphate (NFbHP) media with or without addition of 500 uM potassium phosphate. In these experiments, each pstS and Pliar53 promoter was fused to the reporter gene GFP on a plasmid also carrying a constitutively expressed LSSmScarlett reporter gene such that LSSmScarlett fluorescence could be used to track cell density and internally standardize GFP fluorescence as relative expression units (REU), calculated as GFP fluorescence/LSSmScarlett fluorescence (FIG. 1). In all strains tested, expression was activated from the pstS and Pliar53 promoters greater than 10-fold in the absence of Pi compared to where Pi was added into the media (FIGS. 2-3).

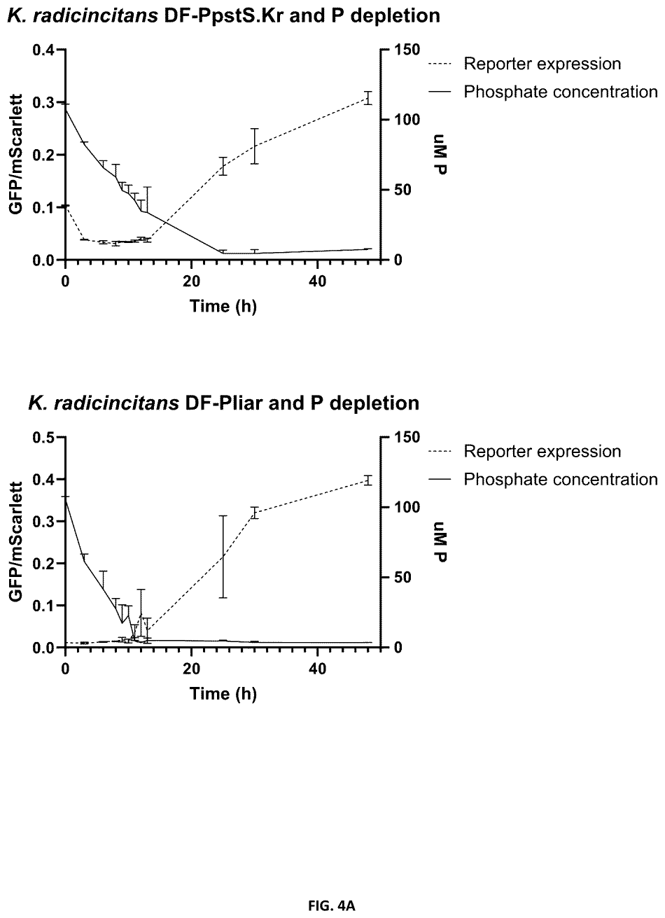

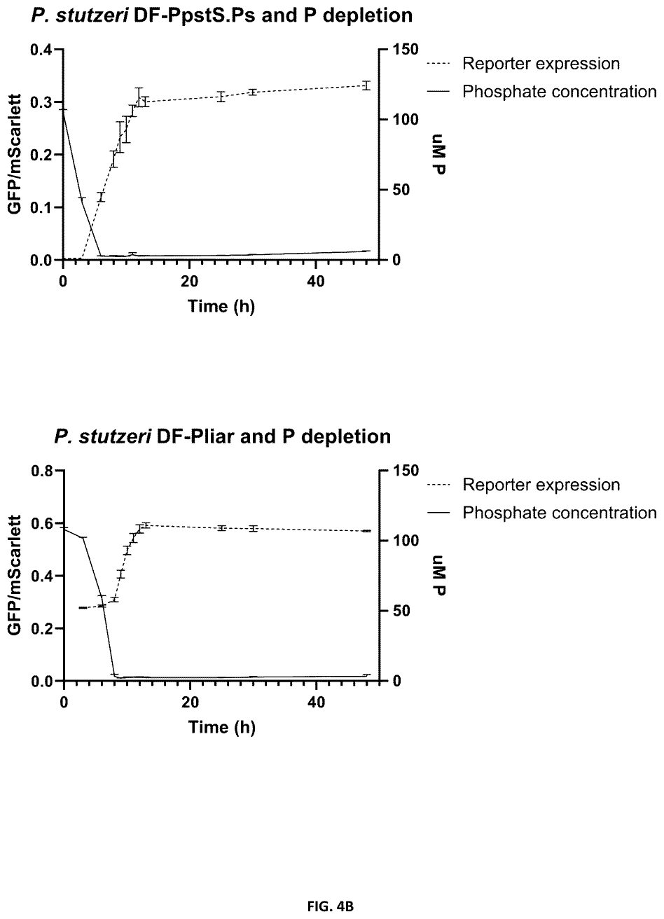

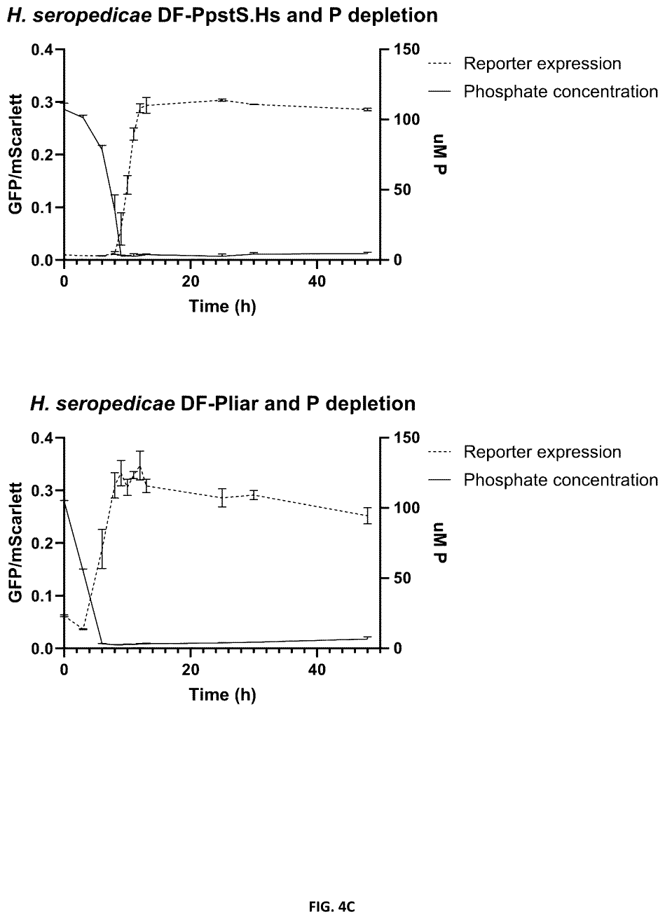

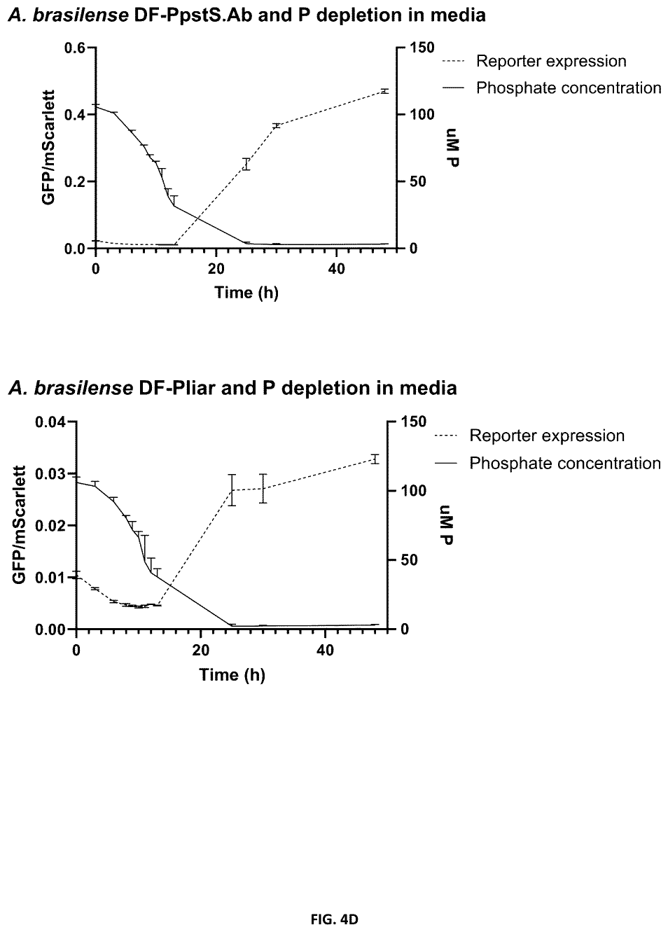

To test whether depletion of Pi in the growth media over time would result in activation of pstS and Pliar53 promoters in Azospirillum brasilense Sp245, Herbaspirillum seropedicae Z67, Pseudomonas stutzeri DSM 4166, and Kosakonia radicincitans DSM16656, cultures (n=3) were thrice washed and inoculated into flat bottomed 96-well plates containing 100 μL of NFbHP supplemented with 100 uM starter potassium phosphate. Cultures were incubated with shaking and GFP/LSSmScarlett fluorescence was read at defined time intervals. In these experiments, each pstS and Pliar53 promoter was fused to the reporter gene GFP on a plasmid also carrying a constitutively expressed LSSmScarlett reporter gene such that LSSmScarlett fluorescence could be used to track cell density and internally standardize GFP fluorescence as relative expression units (REU), calculated as GFP fluorescence/LSSmScarlett fluorescence (FIG. 1). At each interval, cultures were pelleted by centrifugation after and Pi concentration was measured in the cleared supernatant using a Sigma Aldrich Phosphate Assay Kit (Cat MAK308), as per the manufacturer recommendations. In all strains tested, Pi was completely depleted in the media between 0-24 h, whereas, expression was induced in all cases only once the Pi concentration dropped below 50 uM (FIGS. 4A, B, C, and D). Greatest phosphate-sensitive activation of the GFP reporter gene occurred when native pstS promoters derived from each tested strain were used as the control element in the heterologous gene expression cassette, and the heterologous gene expression cassette was integrated at a chromosomal locus distinct from the locus of the endogenous phosphate-sensitive promoter.

### Example 3. Inducing Reporter Gene Expression in Model Soil Bacteria in Response to Decreasing Phosphate Concentration in a Plant Growth Medium

Promoters induced by low Pi concentration activated GFP expression on plant roots following Pi depletion in a sterile sand plant growth medium. Surface sterilized corn seeds were sowed into sterilized 250 mL Schott bottles filled with 200 g of washed sand and 30 mL of Pi-free Hoagland solution. Seeds were germinated in the Schott bottles for 3 days in a growth room set with the following parameters: light intensity 250 μmol/m²/s at 7 inches from the floor; 16:8 light cycles with 30 minute sunrise/sunset dimming; 50-55% humidity. Plants were then inoculated with 1×10⁵ cells of A. brasilense Sp245 carrying a PstS::GFP reporter plasmid (EXAMPLE 2, FIG. 1) with constitutive LSSmScarlett reporter, as confirmed by viable CFU counts. The inoculant cells were prepared by sub-culturing single colonies into NFbHP media with 50 mM potassium phosphate to repress PstS::GFP, 20 mM ammonium chloride, and relevant antibiotics, then incubating overnight at 30° C. with shaking. The following day, cells were harvested by centrifugation and washed twice in NFbHP and resuspended in NFbHP containing 500 uM potassium phosphate to repress the PstS::GFP reporter and provide starter Pi for the plant experiment. After inoculation, plants were incubated in the growth room and sampled at 1, 3, 6, 20, and 40 days post inoculation (dpi). For each sampling point, three plant replicates were destructively uprooted and loose sand was discarded. The roots were then excised from the shoot at the cotyledon and vortexed in water to create a bacterial cell suspension. This fraction was defined in these experiments as the rhizosphere. To measure Pi concentration in the samples, aliquots of the cell suspensions were centrifuged at high speed to remove all solids, and the cleared supernatant was assayed using a Sigma Aldrich Phosphate Assay Kit (Cat MAK308). To isolate bacterial cells for analysis of the PstS::GFP reporter, plant and sand debris remaining in the rhizosphere suspension were selectively cleared by centrifugation at low speed, and one hundred microlitre aliquots of the supernatant containing the bacterial cells were analyzed by flow cytometry.

Because the PstS::GFP reporter plasmid carried a constitutively expressed LSSmScarlett reporter protein, flow cytometry events corresponding to bacterial cells could be gated based on LSSmScarlett fluorescence above the background (here defined as LSSmScarlett+), as validated by Haskett et al. (doi: 10.3389/fmicb.2021.690439). The percentage of the LSSmScarlett+ population activated for PstS::GFP expression at each time point was quantified by establishing PstS::GFP “−” and “+” threshold fluorescence values for the bacteria. This was done by supplementing one experimental treatment with 50 mM potassium phosphate at the time of inoculation to repress expression from PstS::GFP reporter, then generating the GFP−/+ threshold value as the upper 99th percentile of GFP fluorescence in this repressed state, as validated by Haskett et al. (doi: 10.1073/pnas.2117465119).

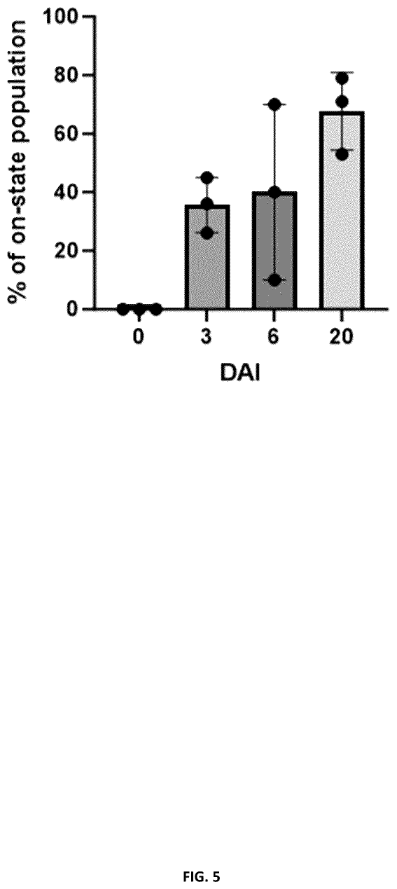

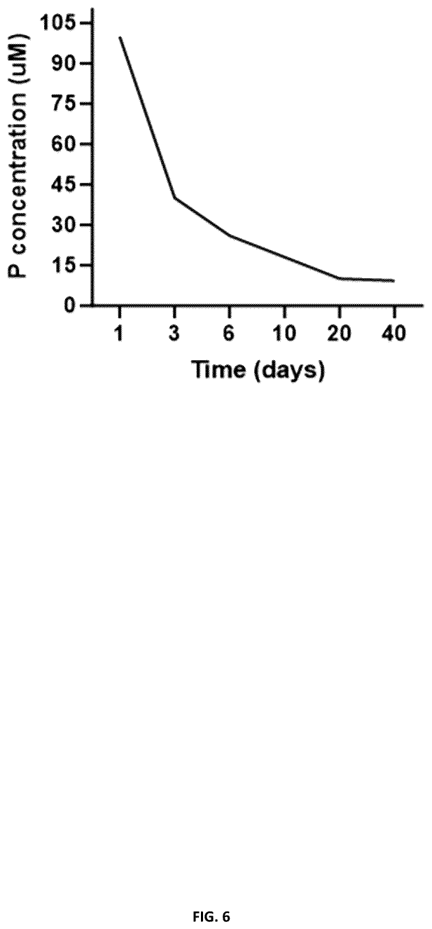

As shown in FIG. 5 and FIG. 6, after 1-dpi, the phosphate concentration in the rhizosphere was determined to be approximately 100 uM, however, the bacterial cell density at this time point was below the detection range for analysis by flow cytometry. After 3-dpi, the phosphate concentration in the rhizosphere fell to 40 uM, triggering a fraction of about 35% of the bacterial population to activate PstS::GFP and enter the GFP+ state. The concentration of Pi continued to decline to about 10 uM by 20-dpi, at which point approximately 70% of the bacterial population were expressing the PstS::GFP reporter fusion, entering the GFP+ state. By 40-dpi, the Pi concentration remained at about 10 uM, and LSSmScarlett+ events were no longer detected in the sample.

### Example 4. Engineering Bacteria to Produce Ammonia with Unidirectional Glutamine Synthetase Adenylyltransferase in Response to Decreasing Phosphate Concentration

Diazotrophic bacteria encode the nitrogenase enzyme capable of reducing inert atmospheric di-nitrogen gas into ammonia under micro-aerobic conditions, providing an autonomous source of nitrogen to the cell. In natural diazotrophs, ammonia derived from nitrogen fixation is assimilated rapidly through the biosynthetic action of glutamine synthetase (GS), which catalyzes the reaction of ammonia and glutamate to form glutamine. Glutamine acts as an indicator of intracellular nitrogen status and blocks expression and activity of nitrogenase on multiple genetic levels. This negative feedback repression prevents diazotrophs from producing more ammonia than is needed for assimilation and prevents release of ammonia for use by plants.

Blocking the activity of GS drives ammonia release in diazotrophs, because a) inability to produce glutamine alleviates negative feedback repression on nitrogenase, and b) inability to assimilate ammonia results in diffusion from the cell. GS activity is modulated through post-translational modification, specifically adenylylation. GS biosynthetic activity can be inhibited by manipulating the adenylylation state. Fully adenylylated GS is inactive but can be de-adenylylated to restore function. In bacteria, adenylylation and deadenylylation of GS is controlled by the bidirectional adenylyltransferase GlnE. Replacement of the glnE gene with a truncated form that carries only the domain required for adenylylation can be used to inactivate GS and drive ammonia release. Ammonia production in response to decreasing phosphate concentration can be caused by operably linking a control element comprising an inducible promoter triggered by low Pi concentration to such a truncated GlnE protein lacking an adenylyl removing domain which exhibits unidirectional adenylyltransferase (uAT) activity.

To generate strains able to release ammonia with uATs in response to decreasing phosphate concentration, the pstS promoter regions of Azospirillum brasilense Sp245 (SEQ ID NO: 418), Herbaspirillum seropedicae Z67 (SEQ ID NO: 422), Pseudomonas stutzeri DSM 4166 (SEQ ID NO: 428), and Kosakonia radicincitans DSM16656 (SEQ ID NO: 423-424) were PCR amplified. Plasmids were designed to encode a uAT (SEQ ID NO: 1) protein of interest operably linked to three different ribosome binding sites (BCD2: strong (SEQ ID NO: 430); BCD22: weak (SEQ ID NO: 449); BCD17: medium strength (SEQ ID NO: 444)) with no promoter, facilitating incorporation of different control elements upstream of the uAT gene. The plasmids were digested with NotI and ligated to the amplified pstS promoters. Assembled plasmids containing complete heterologous gene expression cassettes (FIG. 7) were introduced into Azospirillum brasilense Sp245, Herbaspirillum seropedicae Z67, Pseudomonas stutzeri DSM 4166, and Kosakonia radicincitans DSM16656 host strains with a deletion of the endogenous glnE gene. The expression cassettes were integrated into the chromosome in single copy downstream of each host strain's glmS gene via site-specific Tn7 transposon-mediated recombination. Strains comprising successfully integrated gene expression cassettes were selected via gentamicin resistance, and chromosomal cassette integration was validated by PCR using primers that anneal to junction of the chromosomal DNA and the inserted heterologous gene expression cassettes.

Genetic circuits containing ribosome binding sites of varying strength were tested because uAT expression can reduce glutamine synthetase (GS) activity to a level that is not compatible with cell survival. The level of uAT expression in each strain sufficient to cause ammonia release without impairing cell viability was ascertained.

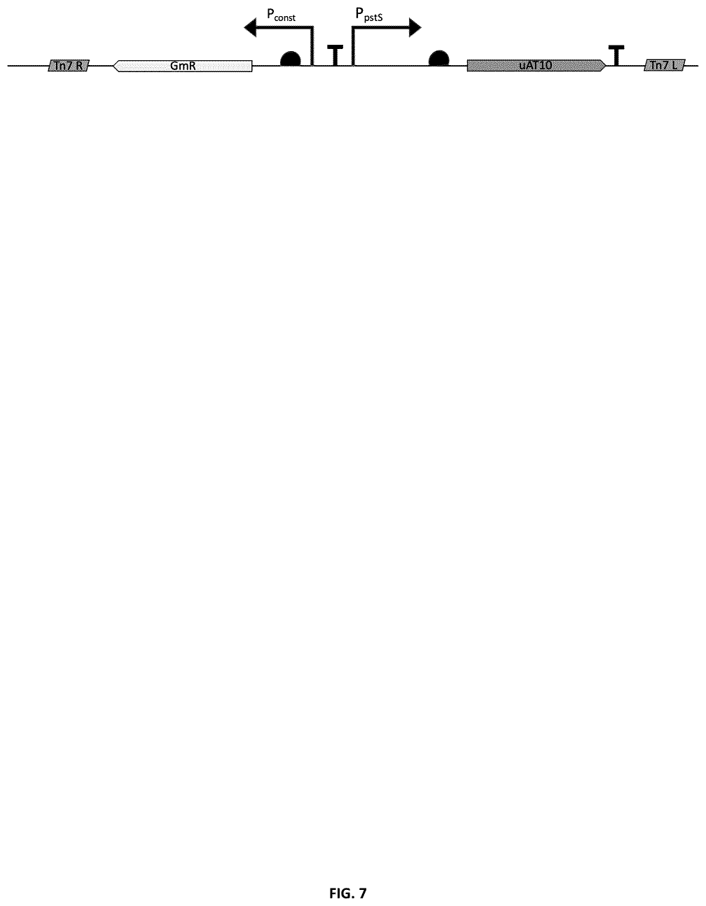

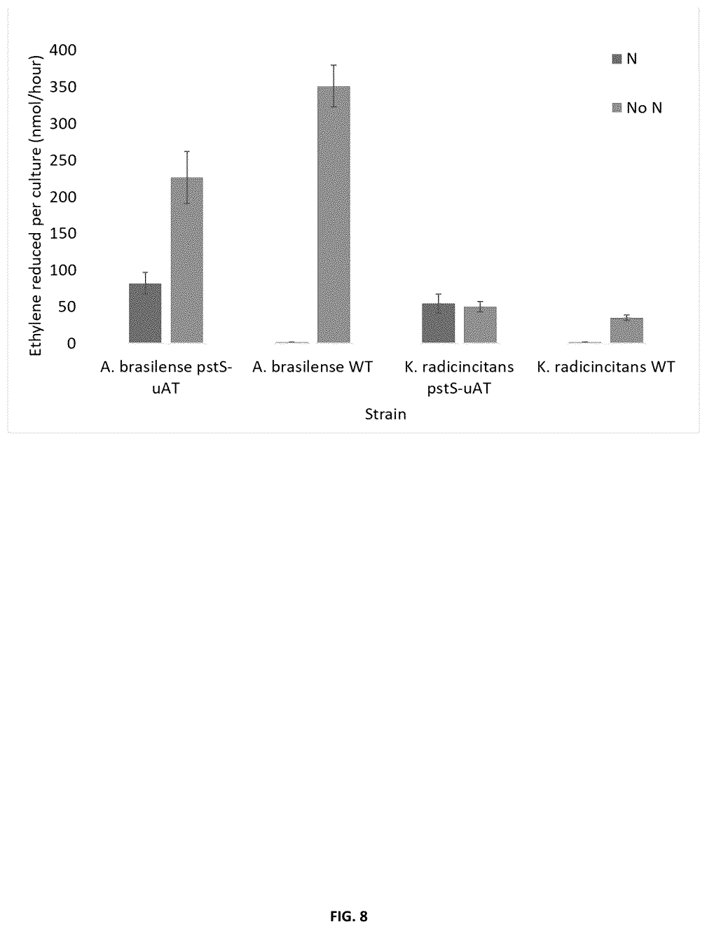

The genetically engineered Azospirillum, Herbaspirillum, Kosakonia, and Pseudomonas strains with the PstS::uA T10 gene expression cassettes (described above) were assayed for de-repression of nitrogenase activity under low phosphate conditions using an acetylene reduction assay. The acetylene reduction assay measures conversion of acetylene to ethylene by nitrogenase as a proxy for conversion of di-nitrogen gas into ammonia. In this experiment, engineered and wild-type bacteria were grown on LB agar plates. Cells were scraped from each plate and resuspended in 22 mL gas chromatography vials at OD600 0.3 in 3 mL of Pi-free semi-solid NFbHP media with either 10 mM ammonium chloride or no added nitrogen. Cultures were sealed by crimping with septum caps and incubated at 30° C. for 24 h prior to starting the assay. The assay was initiated by replacing 10% of the headspace with acetylene gas using a fine gauge needle. Cultures were incubated for an additional 24 h, and ethylene production was quantified in the headspace of each vial using an Agilent 8860 Gas Chromatograph fitted with autosampler (FIG. 8). Wild-type bacteria reduced acetylene into ethylene when cultured in nitrogen-free conditions, but they did not reduce acetylene into ethylene in the presence of 10 mM ammonium chloride. This phenotype was expected because wild-type diazotrophs expressing glutamine synthetase rapidly convert ammonia into glutamine, which represses transcription and activity of nitrogenase. In contrast, glnE mutant bacteria carrying PstS::uAT10 gene expression cassettes reduced acetylene into ethylene both in the absence and presence of ammonium chloride, indicating that GS was inactivated, ammonia was not converted to glutamine, and cells escaped negative feedback repression of nitrogenase.

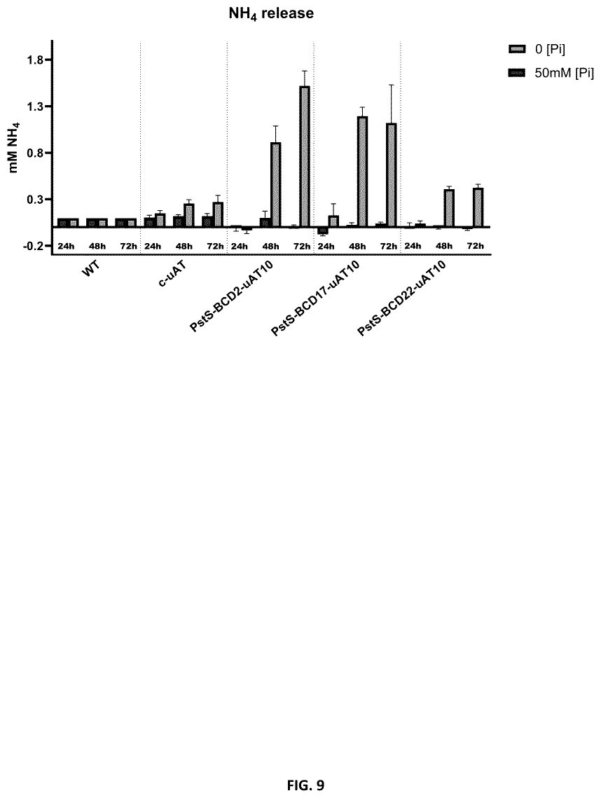

The three genetically engineered Azospirillum brasilense Sp245 strains with the PstS::uAT10 gene expression cassettes containing ribosome binding sites of varying strength were assayed for production of ammonia in the setting of low phosphate concentration. Cells were inoculated in culture tubes at OD600 0.3 into 3 mL semi-solid NFbHP media with or without 50 mM potassium phosphate, and incubated for 72 h at 30° C. 100 μL aliquots of media were sampled from below the pellicle at 24 h time points, centrifuged to pellet agar and cells, and measured for ammonia concentration in the cleared supernatant using an indophenol assay adapted from Schnabel and Sattely (doi: 10.1128/AEM.00582-21). Engineered strains comprising PstS:uA T10 gene expression cassettes were compared to Azospirillum brasilense constitutively expressing uAT10 (c-uAT strain in Schnabel and Sattely (doi: 10.1021/acssynbio.1c00287)) and wild-type A. brasilense Sp245 (FIG. 9).

As expected, wild-type A. brasilense Sp245 did not release ammonia in any condition, and Azospirillum brasilense constitutively expressing uAT10 released a small amount of ammonia at 48 h. All three genetically engineered Azospirillum brasilense Sp245 strains with the PstS::uA T10 gene expression cassettes released ammonia after 48 h in the absence of Pi, but did not release ammonia in the presence of Pi. Strains where the uAT10 gene expressed from the pstS promoter was operably linked to a strong- (BCD 2) or medium-strength (BCD 17) ribosome binding site released approximately two-fold the amount of ammonia compared to the strain where a weak ribosome binding site (BCD 22) was utilized, showing that stronger uAT expression increased ammonia release.

### Example 5. Producing Ammonia from Engineered Bacteria by Overexpressing a GlnE Protein Lacking an Adenylyl Removing Domain

Intracellular glutamine concentration is the dominant indicator of nitrogen status in many nitrogen-fixing bacteria. Decreasing intracellular glutamine concentration prevents cells from sensing high ammonia levels in the environment. Intracellular glutamine concentration can be decreased by increasing expression or activity of glutaminase, an enzyme that converts glutamine into glutamate. Separately, intracellular glutamine concentration can be lowered by decreasing expression or activity of glutamine synthase (GS), an enzyme encoded by the gene glnA that converts ammonia into glutamine. In nitrogen-fixing bacteria, fixed ammonia is quickly assimilated into glutamine and glutamate shunted into cellular metabolism. Blocking ammonia assimilation causes fixed nitrogen to be released into the environment.

Most ammonia is assimilated into glutamine by glutamine synthetase (GS), and subsequently into glutamate by glutamine oxoglutarate aminotransferase (GOGAT). GS is regulated post-translationally by GS adenylyltransferase (GlnE), a bidirectional enzyme encoded by the glnE gene that catalyzes both the adenylylation and de-adenylylation of GS through activity of its adenylyltransferase (AT) and adenylyl-removing (AR) domains, respectively. When nitrogen is abundant, GS is not expressed, and the GlnE AT domain adenylates and deactivates GS. When nitrogen is scarce, GS is expressed, and the GlnE AR domain de-adenylylates and activates GS.

GS adenylyltransferase can be engineered such that it only adenylates and deactivates GS, lacking the ability to reactivate the enzyme. This change in function is achieved by expressing a truncated variant of the GlnE enzyme that lacks the AR domain and regulatory region of the enzyme. Such a truncated GlnE is termed a unidirectional adenylyltransferase (uAT). When a uAT is overexpressed in nitrogen-fixing bacteria, uAT activity deactivates GS independently of the concentration of ammonia, glutamine, and other regulators of nitrogen assimilation. Reduced GS activity in the cells causes a decrease in intracellular glutamine concentration and increase in intracellular ammonia concentration. Cells interpret decreased intracellular glutamine concentration as a need for additional nitrogen fixation, further increasing the intracellular concentration of ammonia through increased fixation of N₂ to NH₃ by nitrogenase. The excess intracellular ammonia is then secreted into the surrounding environment for uptake and assimilation by nearby plants and microbes.

Unidirectional ATases (uATs) are engineered as follows. Where present, the glnE gene encoding glutamine synthetase adenylyltransferase (ATase) is identified in soil bacteria such as those of the genera Acetobacter, Acidothermus, Acinetobacter, Agrobacterium, Aromatoleum, Arthrobacter, Azoarcus, Azorhizobium, Azospirillum, Azotobacter, Bacillus, Bifidobacterium, Bradyrhizobium, Burkholderia, Conexibacter, Curtobacterium, Ensifer, Enterobacter, Erwinia, Escherichia, Flavobacterium, Frankia, Gaiella, Gluconacetobacter, Gluconobacter, Herbaspirillum, Klebsiella, Kosakonia, Lactobacillus, Lactococcus, Lysinibacillus, Maritimibacter, Methylobacterium, Nitrosocosmicus, Nitrososphaera, Paenarthrobacter, Paenibacillus, Panotea, Pediococcus, Peribacillus, Phytobacter, Priestia, Pseudarthrobacter, Pseudomonas, Rahnella, Rhizobium, Rhodococcus, Rhodoplanes, Rhodopseudomonas, Rhodospirillum, Serratia, Solirubrobacter, Sphingobacterium, Sphingomonas, Stenotrophomonas, Streptomyces, Stutzerimonas, Variovorax, Xanthobacter, and Yoonia. Next, the locations of the adenylyl-transferring (AT) and adenylyl-removing (AR) domains within each ATase are predicted. By one method, since the adenylyl-transferring (AT) and adenylyl-removing (AR) domains within ATase are homologous to each other, their locations are predicted through alignment of the N- and C-terminal halves of ATase to each other before designing uAT variants based on this intraprotein homology. By way of a second distinct method, the ATase amino acid sequence in one organism is aligned to that of another to identify the AT and AR domains. After AT and AR domains are identified, a series of uAT candidates is created based on each ATase where each uAT candidate in the series is obtained by selecting a different truncation site in the region between the AR and AT domain (SEQ ID NO: 1-25). The native glnE gene in each organism is then scarlessly deleted by double homologous recombination or other methods, and candidate uATs are expressed. Ammonia release is measured by growing strains in nitrogen-free liquid minimal media with an atmosphere of less than 3% oxygen, or by growing them in nitrogen-free semi-solid agar where bacteria form a pellicle at an oxygen concentration that permits nitrogenase activity. After incubation under these conditions, ammonia is then quantified in samples of cleared supernatant using an ammonium probe or a colorimetric assay such as the indophenol assay described by Schnabel and Sattely (doi: 10.1128/AEM.00582-21).

### Example 6—Producing Ammonia from Engineered Bacteria by Overexpressing Proteins from Nitrogen Fixation and Assimilation Pathways

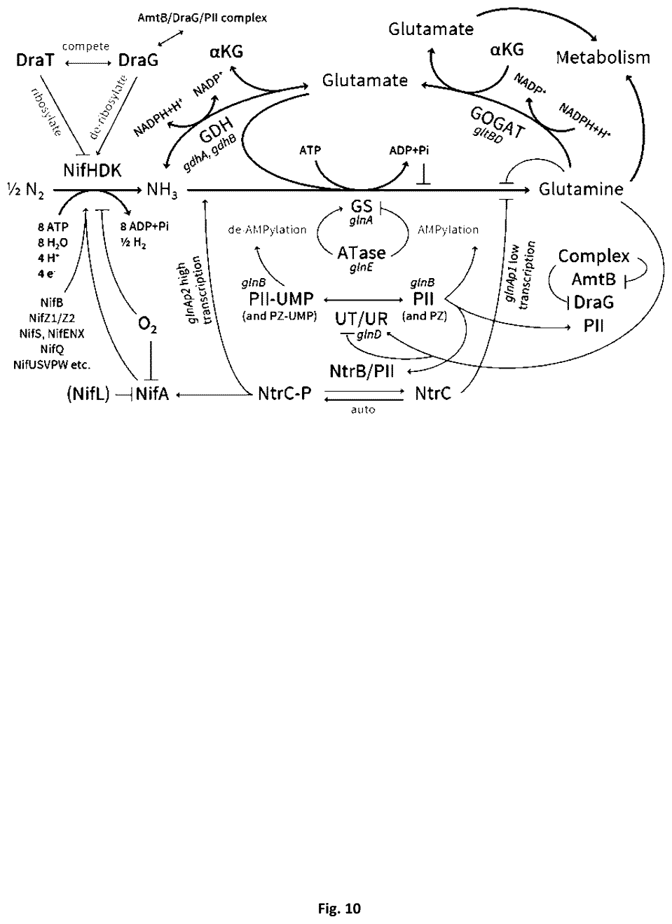

Since nitrogen fixation consumes at least 16 mol of adenosine triphosphate (ATP) per mol N₂ fixed, diazotrophic bacteria have evolved complex regulatory networks to control this energy-intensive metabolic process. To conserve energy, the N₂-fixing catalyst nitrogenase is expressed only under conditions of nitrogen starvation, and the same conditions stimulate upregulation of high-affinity ammonia assimilation by the enzyme glutamine synthetase (GlnA, GS), preventing release of excess ammonia for plants (FIG. 10). Diazotrophs can be engineered to produce and release ammonia by decoupling their ability to repress nitrogenase under nitrogen replete conditions. De-repression of nitrogenase can be achieved by a number of genetic strategies, and in many bacteria, results in more ammonia produced than can be assimilated, causing diffusion of ammonia from the cell.

A distinct method to stimulate ammonia production and release is to prevent bacteria from assimilating fixed nitrogen derived from N₂ into glutamine, the intracellular signal for nitrogen status. This can be accomplished by chemically or genetically inactivating glutamine synthetase by a number of strategies. In addition to preventing assimilation of ammonia derived from nitrogen fixation, low glutamine levels force the cell to engage a nitrogen starvation response, driving de-repression of nitrogenase. High levels of ammonia production paired with inability to assimilate it causes diffusion of ammonia from the cell in high concentrations.

Whether a) nitrogenase feedback repression is alleviated, b) ammonia assimilation and glutamine production is inhibited or c) both processes are engineered, bacteria that produce and release large quantities of ammonia suffer a marked fitness defect that renders them non-competitive and unable to persist in the environment. Thus, inducible ammonia production releases a greater amount of ammonia from an initial bacterial inoculum than constitutive ammonia production because bacterial growth before ammonia production increases bacterial biomass and overall ammonia generated.

The NifA protein, encoded by the nifA gene, is the master transcriptional regulator of genes involved in nitrogenase assembly, function and maintenance, which are encoded by nif cluster and fix cluster genes. Specifically, NifA acts in association with the sigma factor 654 to drive expression of nif cluster and fix cluster genes. The nifA gene in bacteria can be regulated at the transcriptional and posttranslational level by nitrogen, oxygen, and carbon. In Azotobacter vinelandii, overexpression of nifA from a heterologous promoter drives constitutive nitrogenase activity and stimulates ammonia release.

In nature, intracellular levels of active NifA protein are controlled by two key factors: transcription of the nifLA operon and inhibition of NifA protein activity by protein-protein interaction with the NifL protein. Increasing the transcription level of the nifLA operon leads to a higher intracellular concentration of NifA proteins, which increases expression of nitrogenase, the rate of ammonia production, and resulting ammonia release.

The nifL gene is common amongst gamma-proteobacteria and acts as an anti-activator of the nitrogenase master regulator NifA. In conditions of low oxygen and low glutamine, NifL represses activity of NifA, preventing nitrogenase expression and nitrogen fixation. However, deletions in nifL stop NifA inhibition and stop nitrogenase activity from repression by glutamine. Removal of nitrogenase feedback inhibition leads to ammonia release. A nifL gene deletion therefore will stop production of NifL protein, free NifA from inhibition by protein-protein interaction, and permit ammonia release.

glnR acts as a regulator of nitrogen metabolism genes in gram-positive bacteria. In P. polymyxa WLY78, overexpression of glnR drives nitrogenase activity even in the presence of added nitrogen. As such, overexpression of glnR can drive ammonia release in this strain.

### Example 7—Producing Ammonia from Engineered Bacteria by Reducing Expression of Proteins from Nitrogen Fixation and Assimilation Pathways

Ammonia uptake from the environment can be reduced by decreasing the expression level of AmtB ammonia transporter protein (FIG. 10). In Pseudomonas stutzeri A1501, Azotobacter vinlandii DJ, and Gluconobacter diazotrophicus, deletion of the ammonium uptake system encoded by one or more copies of the amtB gene prevents uptake of ammonium from the extracellular environment, and also results in excretion of ammonia from the cells under diazotrophic growth conditions.

Separately, the PII proteins are global nitrogen response regulators, acting on a suite of nitrogen metabolism proteins. In Rhodobacter capsulatus and Azorhizobium caulinodans ORS 571, deletion of both PII proteins forces the adenylyl transferase (AT) to adenylate the GS protein, leading to de-repressed nitrogenase activity in the presence of ammonia. When both PII genes are deleted, ORS 571 releases ammonia into the growth media when grown under nitrogen fixing conditions.

Separately, nitrogenase is regulated by feedback repression at the transcriptional and post translational level by ADP-ribosylation via the DraT-DraG system. DraT catalyzes the ADP-ribosylation of the nitrogenase Fe protein and shuts off of nitrogenase under nitrogen excessive conditions, whereas DraG catalyzes the removal of ADP ribose and reactivation of nitrogenase under nitrogen starvation. Deletion of DraT in a cell where nitrogenase is not regulated by feedback repression at the transcriptional level results in nitrogenase activity that escapes feedback repression.

### Example 8—Producing Ammonia from Engineered Bacteria by Inducibly Repressing Glutamine Synthetase Expression

As described in EXAMPLE 5, down-regulating glutamine synthetase (GS) activity blocks bacteria from assimilating fixed nitrogen and results in ammonia release into the environment. Transcriptionally repressing glnA in response to depletion of phosphate can down-regulate GS and lead to conditional ammonia release under phosphate-limited conditions.

To transcriptionally repress the native glutamine synthetase gene, the native glnA in soil bacteria such as those of the genera Acetobacter, Acidothermus, Acinetobacter, Agrobacterium, Aromatoleum, Arthrobacter, Azoarcus, Azorhizobium, Azospirillum, Azotobacter, Bacillus, Bifidobacterium, Bradyrhizobium, Burkholderia, Conexibacter, Curtobacterium, Ensifer, Enterobacter, Erwinia, Escherichia, Flavobacterium, Frankia, Gaiella, Gluconacetobacter, Gluconobacter, Herbaspirillum, Klebsiella, Kosakonia, Lactobacillus, Lactococcus, Lysinibacillus, Maritimibacter, Methylobacterium, Nitrosocosmicus, Nitrososphaera, Paenarthrobacter, Paenibacillus, Panotea, Pediococcus, Peribacillus, Phytobacter, Priestia, Pseudarthrobacter, Pseudomonas, Rahnella, Rhizobium, Rhodococcus, Rhodoplanes, Rhodopseudomonas, Rhodospirillum, Serratia, Solirubrobacter, Sphingobacterium, Sphingomonas, Stenotrophomonas, Streptomyces, Stutzerimonas, Variovorax, Xanthobacter, and/or Yoonia (SEQ ID NO: 229, 230, 460-511, and 512) is operably linked to a repressible promoter, such as the Ptet promoter (SEQ ID NO: 32), the Plac promoter (SEQ ID NO: 33), or the PphlA promoter (SEQ ID NO: 34). A protein-coding sequence encoding a repressor protein paired with the repressible promoter, TetR (SEQ ID NO: 27), LacI (SEQ ID NO: 29), or PhlF (SEQ ID NO: 30) respectively, is operably linked to a phosphate-sensitive promoter (SEQ ID NO: 417-428) in a heterologous gene expression cassette, which is introduced into a soil bacterium to create a genetically engineered bacterium. The genetically engineered bacterium (GEB) is fermented in high-phosphate media such that the bacteria grow to high cell density. The GEB are then introduced into a plant growth medium in an agricultural context. Once the phosphate concentration in the plant growth medium falls below a threshold level (e.g. about 50 μM, 40 μM, 30 μM, 20 μM, or 10 μM to about 1 μM to about 1 μM), the repressor protein is expressed from the phosphate sensitive promoter, binds to the repressible promoter, and down-regulates expression of glnA, leading to ammonia release.

### Example 9—Producing Ammonia from Engineered Bacteria by Inducibly Cleaving an Engineered Glutamine Synthetase to Abrogate Enzymatic Activity

Glutamine synthetase (GS) is catalytically inactivated by disrupting the structure of its active site through cleavage of the GS peptide backbone. Hydrolysis of the GS peptide backbone can be catalyzed by proteases, a class of enzymes that recognize specific short amino acid sequences and cleave a protein's peptide backbone a consistent distance from said amino acid recognition sequences, herein referred to as protease-specific recognition sequences (PSRS). A GS protein variant that is controllably cleaved by a protease is engineered by encoding one or more PSRS within the coding sequence of the glnA gene. By engineering a nitrogen-fixing bacterial strain to contain (i) a GS protein comprising one or more PSRS and (ii) the cognate protease under inducible control, GS is catalytically inactivated in response to environmental changes. In this system, introduction of an environmental stimulus induces expression of a protease. The protease then binds the one or more PSRS in the engineered GS and cleaves the GS backbone, causing a structural change in GS that inactivates it catalytically and causes ammonia accumulation due to GS down-regulation.

To engineer an organism with a GS protein that is inactivated by an inducible protease, the GS protein is engineered to comprise one or more PSRS. Proteases capable of cleaving the engineered GS protein peptide backbone include the tobacco etch virus (TEV) protease (SEQ ID NO: 45), tobacco vein mottling virus (TVMV) protease (SEQ ID NO: 46), sunflower mild mosaic virus (SMMV) protease (SEQ ID NO: 47), turnip mosaic virus (TuMV) protease (SEQ ID NO: 48), soybean mosaic virus (SMV) protease (SEQ ID NO: 49), plum pox potyvirus (PPV) protease (SEQ ID NO: 50), Hepatitis C virus (HCV) NS3 protease (SEQ ID NO: 51), Coagulation factor Xa (SEQ ID NO: 52), and Furin (SEQ ID NO: 53). The proteases above recognize the following PSRS sequences, respectively: EXXYXQ-(S/G) or ENLYFQ-(S/G/A/M/C/H), wherein X is any amino acid and the TEV protease cleaves between the Q and the S, G, A, M, C, or H residues; ETVRFQ-(G/S), wherein the TVMV protease cleaves between the Q and S or G residues; EEIHLQ-(S/G), wherein the SMMV protease cleaves between the Q and S or G residues; VXHQ or VRHQ-S, wherein X is any amino acid and the TuMV protease cleaves C-terminal to the Q residue; XVXXQ-(G/S), wherein X is any amino acid and the SMV protease cleaves between Q and S or G residues; NVVVHQ-A, wherein the PPV protease cleaves between the Q and the A residue; (D/E)XXXXC(A/S), wherein X is any amino acid and the HCV NS3 protease cleaves between the C and the A or S residues; I(D/E)GR, wherein the Factor Xa protease cleaves C-terminal to the R residue; or RX(K/R)R, wherein the furin protease cleaves C-terminal to the C-terminal R residue.

The one or more PSRS are incorporated into the GS coding sequence in a number of locations. To ensure that the one or more PSRS will be efficiently bound by a protease, the PSRS is/are located at the solvent-facing surface of the GS protein to increase the probability that the protease molecule will collide with the PSRS in the GS protein variant and bind to it/them. Multiple methods are used to identify the solvent-facing surface of the GS protein, including visualization of external amino acid residues using crystal structure data, alignment of the amino acid sequence of GS variants without a crystal structure to those with one in order to identify homologous regions of the protein, in silico structure prediction for GS variants without a crystal structure and identification of solvent-facing regions of the protein, and mass-spectrometry based protein labeling methods that identify solvent-facing regions of a protein. Additionally, catalytic deactivation of GS is more complete if protease cleavage compromises the structure of the GS enzyme active site. Compromising the structure of the active site is achieved by rationally positioning the PSRS such that cleavage will create multiple protein fragments that each contain a portion of the incomplete active site, rationally positioning the one or more PSRS near or within the active site so that the break in the peptide backbone alters the sterics of the active site, or screening the enzymatic activity of a library of GS variants with the PSRS positioned at random locations.

In the Azospirillum brasilense Sp245 GS polypeptide (SEQ ID NO: 229), locations to incorporate one or more PSRS that meet the above criteria include between amino acid residues 98 and 99, 121 and 122, 279 and 280, and/or 285 and 286. In the Pseudomonas stutzeri GS polypeptide (SEQ ID NO: 230), locations to incorporate one or more PSRS that meet the above criteria include between amino acid residues 98 and 99, 119 and 120, 283 and 284, and/or 298 and 299. These insertion sites are depicted in Table 6.

To achieve inducible deactivation of GS, GS must function normally until an environmental stimulus triggers protease expression, at which point enzymatic activity is minimized. To achieve normal function of GS until the protease is expressed, the native glnA gene in a bacterial strain is replaced with an engineered copy of glnA encoding the one or more PSRS so that the engineered GS is expressed by way of the native glnA genetic context of the host strain. To minimize GS activity after protease cleavage, the engineered copy of glnA encoding the one or more PSRS is the only glnA allele in the host strain. The gene encoding GS, glnA, is an essential gene for many bacterial strains. However, replacing a single native allele with a single engineered allele is accomplished by inserting certain site-specific recombinase recognition sites (SSRRS) flanking a bacterium's native glnA allele in the bacterium's chromosome (including FRT sites and/or loxP sites), encoding the corresponding site-specific recombinase (a yeast flippase (FLP) recombinase if FRT SSRRS or a Cre-recombinase if loxP SSRRS) under inducible control on a plasmid with the glnA allele encoding a GS variant comprising the one or more PSRS, and inducing recombination to swap the engineered glnA allele into the chromosome while excising the native allele. Such a process achieves exchange of the wild-type glnA allele for the engineered glnA allele encoding the GS that comprises the one or more PSRS without ever requiring deletion of the essential glnA gene.

Once a bacterial strain is obtained where the native gene encoding GS has been replaced by a GS gene encoding a GS variant comprising the one or more PSRS, a heterologous gene expression cassette comprising a protease-encoding gene (e.g. SEQ ID NO: 54, 55, or 56) operably linked to a phosphate sensitive promoter (SEQ ID NO: 417-428) is introduced. Since the protease gene is expressed by a phosphate-sensitive promoter, (SEQ ID NO: 417-428), expression of the protease is prevented before the phosphate-sensitive promoter is activated.

A genetically engineered bacterium (GEB) comprising, (i) a gene encoding a GS variant comprising one or more PSRS and (ii) a heterologous gene expression cassette comprising a protease gene of interest operably linked to a phosphate-sensitive promoter, is obtained. Enzymatic activity of a GS variant comprising one or more PSRS within a GEB is compared to activity of wildtype GS in a strain-matched bacterium containing a wild-type GS variant to confirm enzymatic function of the PSRS-containing GS variant, using a biochemical assay. Subsequently the GEB comprising the PSRS-containing GS variant is evaluated for ammonia release via an indophenol assay.

In Azospirillum brasilense Sp245, introducing two PSRS into the natively controlled glnA gene followed by inducing TEV protease expression under control of a tetracycline-responsive promoter via anhydrotetracycline yields a significant decrease in GS activity compared to the wild-type. Placing the engineered glnA alleles under control of constitutive promoters at the native locus (instead of the natively regulated promoter) additionally abolishes up-regulation of glnA expression when GS is cleaved by the proteases.

In its agricultural application, the genetically engineered bacterium (GEB) is fermented in high-phosphate media such that the bacteria grow to high cell density. The GEB are then introduced into a plant growth medium in an agricultural context. Once the phosphate concentration in the plant growth medium falls below a threshold level (e.g. about 50 μM, 40 μM, 30 μM, 20 μM, or 10 μM to about 1 μM to about 1 μM), the protease is expressed from the phosphate sensitive promoter, cleaves the GS variant expressed by the GEB, and down-regulates expression of glnA, leading to ammonia release.

### Example 10. Engineering Bacteria Isolated from Agricultural Fields to Express a Reporter Gene in Response to Decreasing Phosphate Concentration

To generate strains from agricultural soil isolates that express a chromosomally integrated GFP reporter gene in response to phosphate depletion, the pstS promoter regions of soil isolates of Klebsiella variicola (SEQ ID NO: 515), Kosakonia sacchari (SEQ ID NO: 517), and Rahnella aceris (SEQ ID NO: 518) were PCR amplified from genomic DNA, each with HiFi adapters for cloning into an entry plasmid digested with NotI. The cloning site was designed such that the Pi-dependent promoters were positioned upstream of GFP. Plasmids were confirmed by next generation sequencing. Assembled plasmids containing complete heterologous gene expression cassettes (FIG. 1) were introduced into Klebsiella variicola, Kosakonia sacchari, and Rahnella aceris strains isolated from agricultural corn fields. The expression cassettes were integrated into the chromosome in single copy downstream of each host strain's glmS gene via site-specific Tn7 transposon-mediated recombination. Strains comprising successfully integrated gene expression cassettes were selected via gentamicin resistance, and chromosomal cassette integration was validated by PCR using primers that anneal to junction of the chromosomal DNA and the inserted heterologous gene expression cassettes.

### Example 11. Inducing Reporter Gene Expression in Bacteria Isolated from an Agricultural Field in Response to Decreasing Phosphate Concentration in an Agricultural Field Soil Substrate

Promoters induced by low Pi concentration activated GFP expression on corn roots following Pi depletion in agricultural field soils used as a growth medium. Topsoil was collected from within 10 inches of the surface from American commercial maize fields in the states of Illinois (two sites), North Dakota, and Oklahoma (“Field Soil”) and underwent soil chemistry analysis. 2×10″ Deepot cells (Stuewe & Sons, Inc.) were each filled with 300 g of Field Soil from a single location that had been titrated to 100 ppm potassium and 100 ppm nitrate. In one experimental group (“Illinois B+P solution”), 226 L of Anderson's 10-34-0 in furrow fertilizer was also added to each Deepot cell. After 24 hours, surface sterilized corn seeds were sown at a depth of 2 inches and inoculated with either (i) 6.2×10⁷ cells of K. sacchari carrying a GFP reporter expression cassette driven by the K. sacchari pstS promoter (SEQ ID NO: 517) or (ii) 8.7×10⁷ cells of K. variicola carrying a GFP reporter expression cassette driven by the K. variicola pstS promoter (SEQ ID NO: 515), as confirmed by viable CFU counts. After inoculation, plants were incubated in a growth room set with the following parameters: light intensity 250 μmol/m²/s at 7 inches from the floor; 16:8 light cycles with 30 minute sunrise/sunset dimming; 50-55% humidity. Plants were sampled at 7 days (“1 week”) and 14 days (“2 weeks”) post inoculation. For each sampling point, five plant replicates were destructively uprooted and loose field soil was discarded. The roots were then excised from the shoot at the cotyledon and vortexed in phosphate-buffered saline with 10 mM ammonium chloride for 1 hour to create a bacterial cell suspension. This fraction was defined in these experiments as the rhizosphere. To isolate bacterial cells for analysis of the GFP reporter, plant and soil debris remaining in the rhizosphere suspension were selectively cleared by centrifugation at low speed, and one hundred microlitre aliquots of the supernatant containing the bacterial cells were analyzed by flow cytometry.

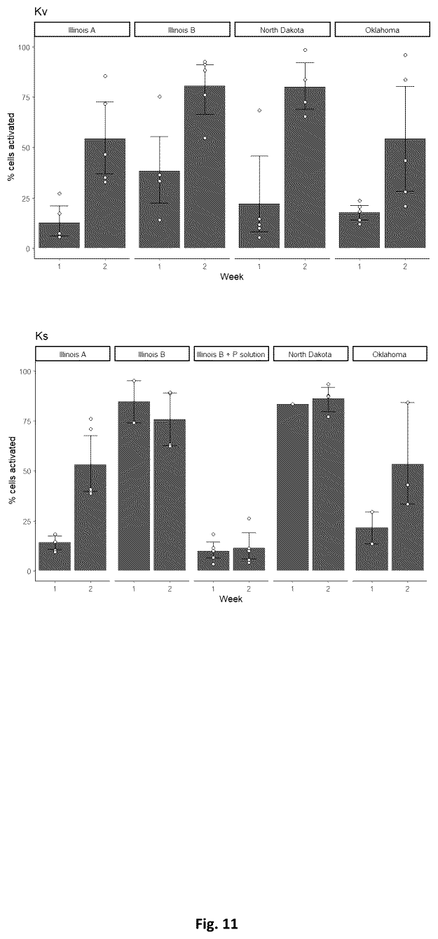

Because the GFP reporter cassette carried a constitutively expressed LSSmScarlett reporter protein, flow cytometry events corresponding to bacterial cells could be gated based on LSSmScarlett fluorescence above the background (here defined as LSSmScarlett+), as validated by Haskett et al. (doi: 10.3389/fmicb.2021.690439). The percentage of the LSSmScarlett+ population activated for GFP expression at each time point was quantified by establishing GFP “−” and “+” threshold fluorescence values for the bacteria. This was done by supplementing one experimental treatment with 50 mM potassium phosphate at the time of inoculation to repress expression from GFP reporter, then generating the GFP−/+ threshold value as the upper 99th percentile of GFP fluorescence in this repressed state, as validated by Haskett et al. (doi: 10.1073/pnas.2117465119). As shown in FIG. 11, engineered K. sacchari demonstrated over 25% activation with a 2 week delay in 2 out of 4 field soil types while engineered K variicola demonstrated from over 25% to over 50% activation with a 2 week delay in 4 out of 4 field soil types. In the group where each sample was treated with extra phosphate by way of an additional 226 L of Anderson's 10-34-0 in furrow fertilizer, the GFP reporter was not expressed above background, even after 2 weeks.

### Example 12. Further Description of Ammonia Production from Engineered Bacteria by

### Inducibly Repressing Glutamine Synthetase Expression

As described in EXAMPLE 8, down-regulating glutamine synthetase (GS) activity blocks bacteria from assimilating fixed nitrogen and results in ammonia release into the environment. Transcriptionally repressing glnA in response to depletion of phosphate can down-regulate GS and lead to conditional ammonia release under phosphate-limited conditions.

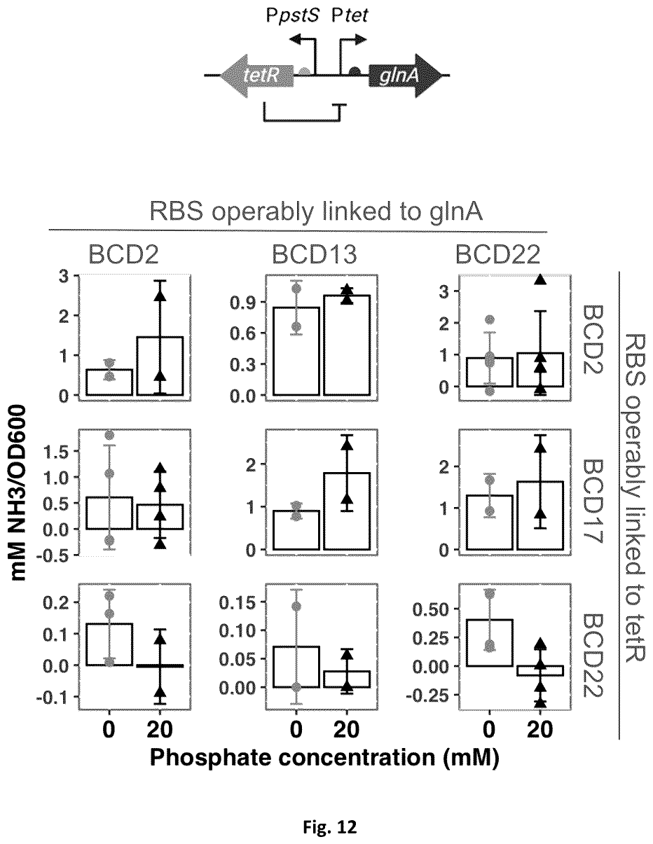

In this example, the native (endogenous) glnA gene encoding GS is deleted, and a cassette is integrated into the chromosome in single copy with the design in the schematic shown in FIG. 12 with one sequence element for each of sites 1-6 as described in Table 9.

TABLE 9
Non-limiting Examples of glnA Cassettes with Sequence Elements Depicted
in FIG. 12: Site 1 (Phosphate-sensitive promoters); Sites 2 and 5 (Ribosome
Binding sites); Site 3 (Encoded Repressor proteins); Site 4 (Repressor-
bound promoters); and Site 6 (encoded glutamine synthase).

| Site | | | SEQ ID | | |
|---|---|---|---|---|---|
| # | Part Type | Description | NO | Type | Organism name |
| 1 | Phosphate- | A. brasilense pstS | 418 | DNA | Azospirillum brasilense |
| | sensitive | promoter | | | |
| | promoter | H. seropedicae pstS | 422 | DNA | Herbaspirillum |
| | | promoter | | | seropedicae |
| | | K. radicincitans pstS | 423 | DNA | Kosakonia radicincitans |
| | | promoter 1 | | | |
| | | P. stutzeri pstS promoter | 428 | DNA | Pseudomonas stutzeri |
| | | K. sacchari pstS promoter | 517 | DNA | Kosakonia sacchari |
| | | K. variicola pstS promoter | 515 | DNA | Klebsiella variicola |
| | | R. aceris pstS promoter | 518 | DNA | Rahnella aquatilis |
| 2, 5 | Ribosome | BCD 2 vL ribosome | 430 | DNA | synthetic construct |
| | binding site | binding site | | | |
| | | BCD 13 ribosome binding | 440 | DNA | synthetic construct |
| | | site | | | |
| | | BCD 17 ribosome binding | 444 | DNA | synthetic construct |
| | | site | | | |
| | | BCD 22 vL ribosome | 449 | DNA | synthetic construct |
| | | binding site | | | |
| 3 | Repressor | Optimized TetR | 516 | PRT | synthetic construct |
| | protein | | | | |
| | encoded | | | | |
| 4 | Repressor- | Ptet promoter (binds TetR | 32 | DNA | synthetic construct |
| | bound | repressors) | | | |
| | promoter | | | | |
| 6 | Glutamine | A. brasilense glutamine | 229 | PRT | Azospirillum brasilense |
| | synthetase | synthetase protein (GlnA) | | | |
| | encoded | K. sacchari glutamine | 519 | PRT | Kosakonia sacchari |
| | | synthetase | | | |
| | | K. variicola glutamine | 520 | PRT | Klebsiella variicola |
| | | synthetase | | | |
| | | R. aceris glutamine | 521 | PRT | Rahnella aceris |
| | | synthetase | | | |

In certain embodiments, other TetR proteins and Ptet promoters or TetR binding sites in those promoters that can be used include those described in Ramos et al., 2005, doi.org/10.1128/mmbr.69.2.326-356.2005.

### Example 13. Ammonia Release is Activated Under Low Pi Conditions for Constructs Made with Heterologous Parts

Systems as described in Example 12 were constructed essentially as described in Example 10 with TetR as a repressor expressed from a K. variicola pstS promoter (SEQ ID NO: 515) and Azospirillum glnA expressed from Ptet promoter and tested for ammonia production in the presence and absence of Pi.

To test for ammonia production, cell mass of each genetically engineered strain of interest was grown on solid Luria-Bertani agar and resuspended in 1% potassium chloride to OD₆₀₀ 3.0. Cells were inoculated at a final OD₆₀₀ of 0.3 into gas chromatography (GC) vials containing liquid HEPES-buffered NFbHP medium supplemented with glucose, with or without 20 mM Pi. To generate microaerobic culture conditions amendable to nitrogen fixation, cultures were incubated shaking for 30 minutes at room temperature in a 2% oxygen atmosphere in a custom-built anaerobic chamber. GC vials were sealed and verified to be air-tight prior to transfer out of the anaerobic chamber. GC vials were subsequently incubated for 24 hours at 30° C. shaking at 200 rpm. A separate 200 μL of each culture was sampled for (i) quantification of ammonia production using a Sigma-Aldrich MAK310 kit according to manufacturer instructions and (ii) quantification of OD₆₀₀ using a spectrophotometric 96-well plate reader.

Constructs (FIG. 12, upper panel) carrying each combination of RBSs (FIG. 12, lower panel) were integrated into the Kosakonia sacchari (Ks) soil isolate and the endogenous glnA gene was deleted from the chromosome. Ammonia release+/−added Pi was measured in vitro as described above (FIG. 12, lower panel). While all combinations display the desirable phenotype of ammonia release in absence of Pi, strains comprising a strong- (BCD2) or medium-strength (BCD17) ribosome binding site operably linked to the tetR gene also display the undesirable phenotype of ammonia release in presence of Pi. Transformed strains exhibiting desirable inducible ammonia release phenotype are displayed in the bottom row of panels of the 3×3 FIG. 12. The relative strength of the RBS used in the various constructs is BCD22<BCD13 BCD17<BCD2. The results of these experiments show that constructs where the tetR gene encoding the TetR is operably linked to a weak RBS (BCD22) provide the best induction of ammonia release under low Pi conditions.

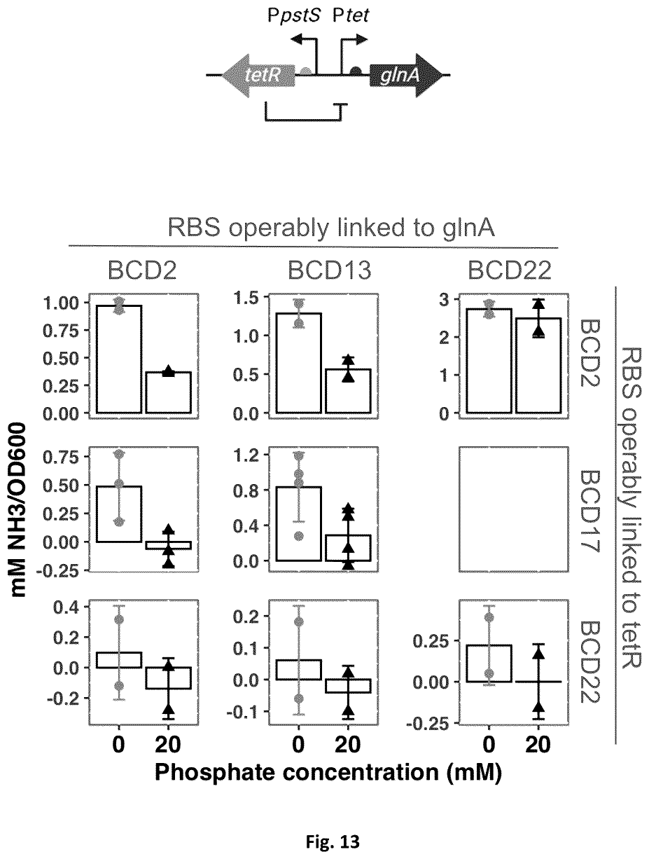

Constructs (FIG. 13, upper panel) carrying each combination of RBSs (FIG. 13, lower panel) were integrated into the Rahnella aceris (Ra) soil isolate, and the endogenous glnA gene was deleted from the chromosome. Ammonia release+/−added Pi was measured in vitro as described above (FIG. 13, lower panel). While all combinations display the desirable phenotype of ammonia release in absence of Pi, strains comprising a strong- (BCD2) ribosome binding site operably linked to the tetR gene also display the undesirable phenotype of leaky ammonia release in presence of Pi. The relative strength of the RBS used in the various constructs is BCD22<BCD13˜BCD17<BCD2. The results of these experiments show that constructs where the tetR gene encoding the TetR is operably linked to a weak RBS (BCD22) or medium-strength RBS (BCD17) provide the best induction of ammonia release under low Pi conditions.

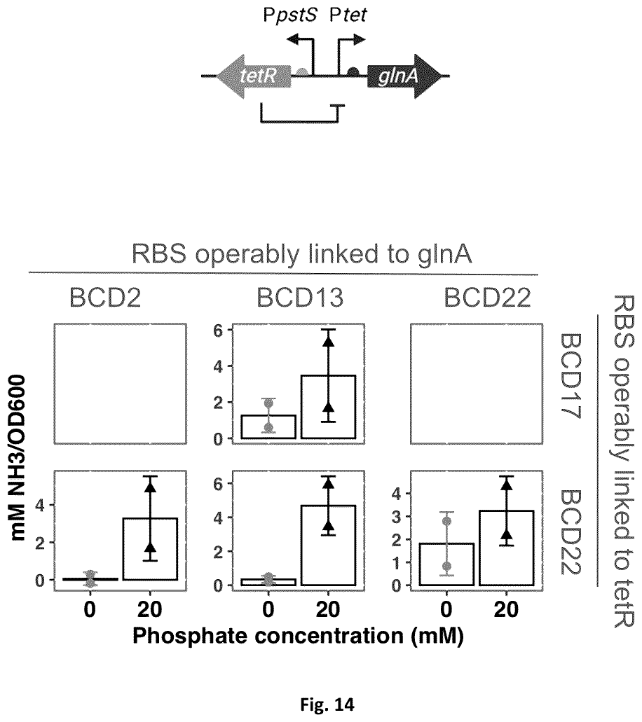

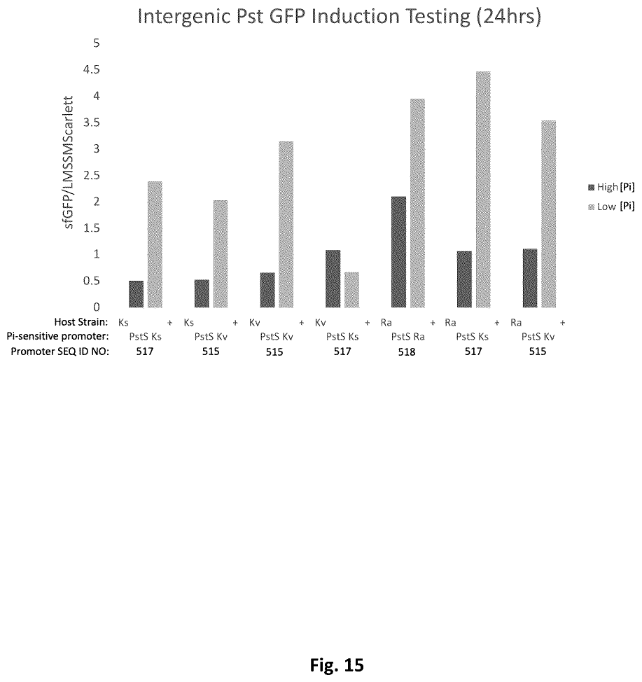

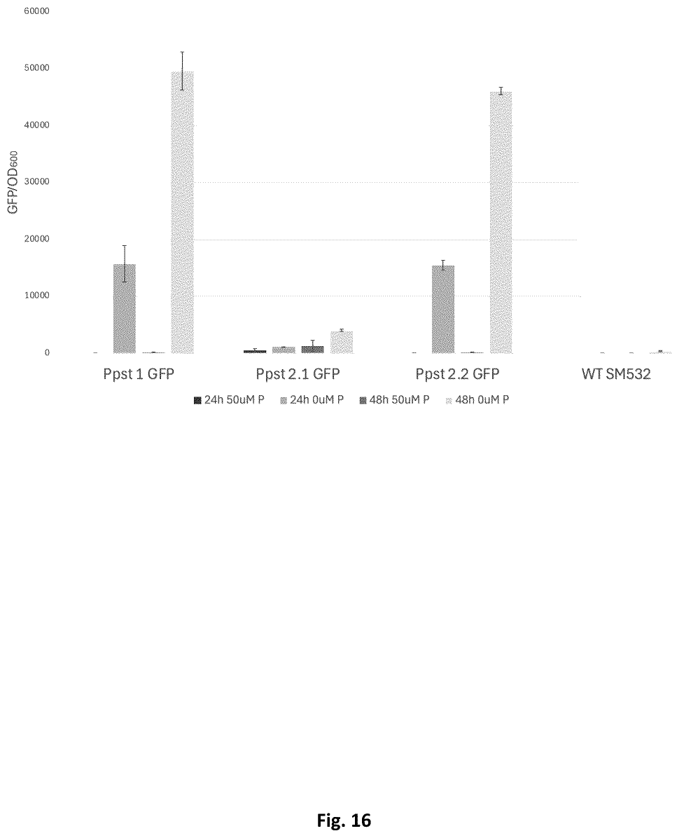

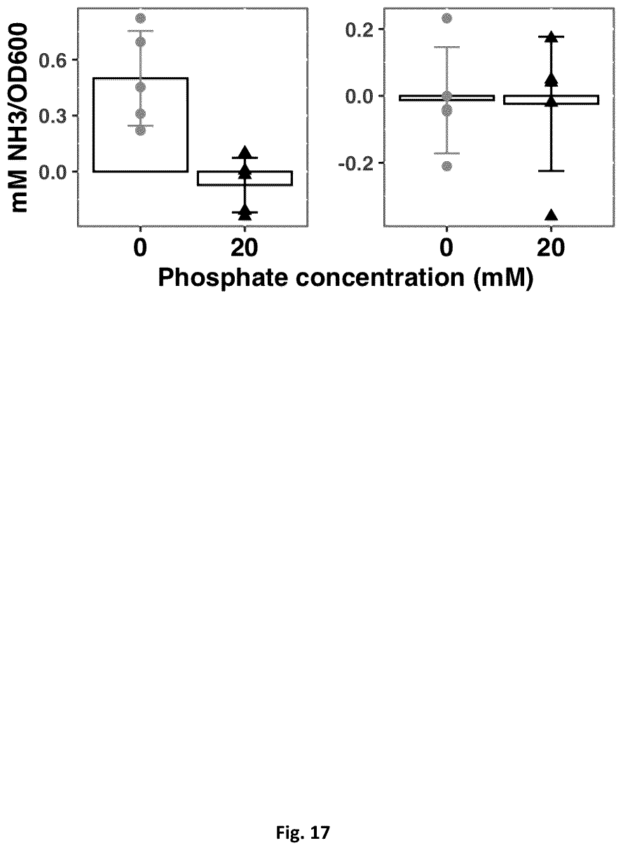

Constructs (FIG. 14, upper panel) carrying each combination of RBSs (FIG. 14, lower panel) were integrated into the Klebsiella variicola (Kv) soil isolate, and the endogenous glnA gene was deleted from the chromosome. Ammonia release+/−added Pi was measured in vitro as described above (FIG. 14, lower panel). None of these strains achieved ammonia production inducible by a reduction in Pi concentration.

### Example 14. PpstS Promoters Activate GFP in Response to Low-Pi in Sterile Cultures of Gram-Negative Gamma-Proteobacteria and Gram-Positive Paenibacillus

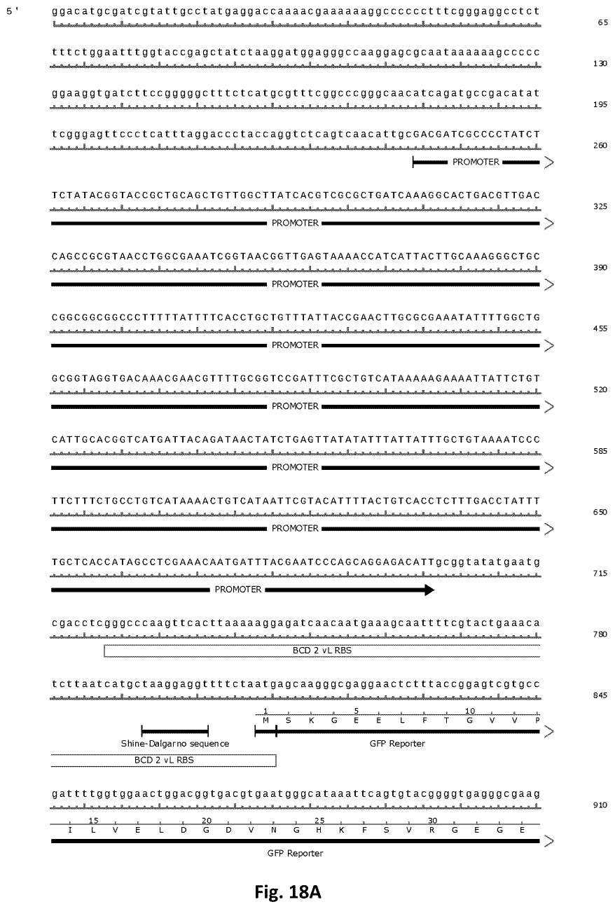

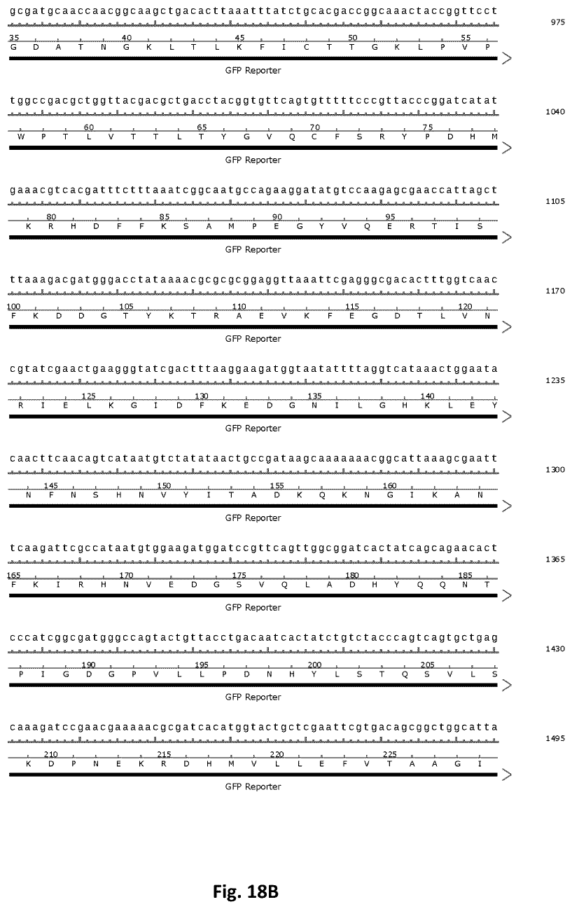

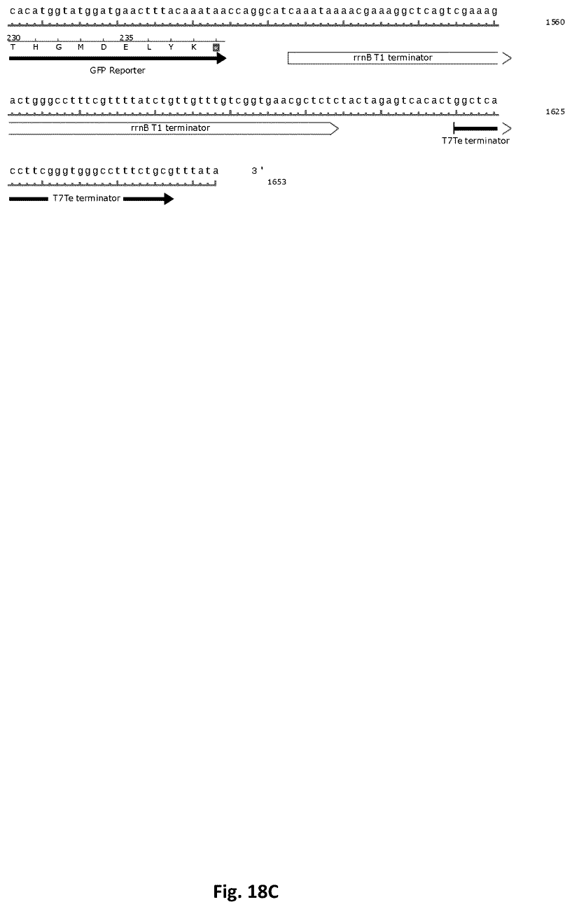

Activation of GFP reporter expression driven by various PpstS promoters in response to low Pi in the genetically engineered bacterial strains of Example 10 was tested in sterile culture essentially as described in Example 2. The promoters that were tested are disclosed in Table 9. Promoters comprising ˜300-bp upstream of the endogenous pstS gene in a Kosakonia sacchari soil isolate, a Klebsiella varicolla soil isolate, and a Rahnella aceris soil isolate activated GFP expression in response to low Pi in response to Pi depletion as shown in FIG. 18. Phosphate sensitive promoters were also tested in Paenibacillus graminis. The PstSI and PstsII promoters for a Paenibacillus graminis soil isolate activate GFP expression in liquid media in response to Pi depletion (Ppst 1, Ppst2.1, and Ppst 2.2 in FIG. 19).

### Example 15. Low Phosphate Control of a Unidirectional Adenylyl Transferase Fused to the PstS Promoter Drives Ammonia Release in Kosakonia sacchari

A Kosakonia sacchari soil isolate was transformed with a vector containing a PstS promoter (SEQ ID NO: 423) fused to DNA encoding a unidirectional adenylyl transferase (SEQ ID NO: 16). Ammonia was released at 0 mM Pi but not at 20 mM Pi as shown in FIG. 20.

What is claimed is:

1. A genetically engineered bacterium comprising:
(i) a first recombinant DNA molecule comprising a phosphate-sensitive promoter of a bacterial pstS phosphate ABC transporter gene operably linked to DNA encoding a transcript comprising a ribosome binding site (RBS) operably linked to a transcriptional repressor protein coding region, wherein the RBS, when substituted in place of nucleotides 723 to 810 of control green fluorescent protein (GFP) reporter gene expression cassette of SEQ ID NO: 522, provides 0.1% to 44% of operably linked GFP reporter gene expression provided by the control GFP reporter gene expression cassette of SEQ ID NO: 522 when expressed under otherwise identical conditions; and
(ii) a second recombinant DNA molecule comprising a promoter which is repressed by the transcriptional repressor protein and which is operably linked to DNA encoding a bacterial glutamine synthetase, wherein the genetically engineered bacterium is of the genus Kosakonia.

2. The bacterium of claim 1, wherein the bacterial pstS phosphate ABC transporter gene is a Klebsiella sp., Klebsiella variicola, Kosakonia sp., or Kosakonia sacchari pstS phosphate ABC transporter gene.

3. The bacterium of claim 1, wherein the bacterial pstS phosphate ABC transporter gene encodes a phosphate ABC transporter protein having at least 76% sequence identity to SEQ ID NO: 513 and comprising the polypeptide of SEQ ID NO: 514.

4. The bacterium of claim 1, wherein the phosphate-sensitive promoter comprises a sequence having at least 95% sequence identity to SEQ ID NO: 515 and comprising at least one Pho box comprising the DNA molecule of SEQ ID NO: 525, 526, 527, 528, and/or 529.

5. The bacterium of claim 1, wherein the transcriptional repressor protein is a bacterial TetR protein.

6. The bacterium of claim 1, wherein the ribosome binding site (RBS) which is operably linked to a transcriptional repressor protein coding region comprises a BCD22vL ribosome binding site encoded by SEQ ID NO: 449.

7. The bacterium of claim 1, wherein transcriptional repressor protein coding region encodes a bacterial TetR transcriptional repressor protein having at least 95% sequence identity to SEQ ID NO: 516.

8. The bacterium of claim 1, wherein transcriptional repressor protein coding region encodes a bacterial TetR transcriptional repressor protein comprising the amino acid sequence of SEQ ID NO: 516.

9. The bacterium of claim 1, wherein the bacterial glutamine synthetase is an Azospirillum glutamine synthetase and the endogenous gene encoding the Kosakonia glutamine synthetase protein contains a null mutation or is deleted.

10. The bacterium of claim 1, wherein the bacterial glutamine synthetase comprises a Kosakonia glutamine synthetase.

11. The bacterium of claim 10, wherein the Kosakonia glutamine synthetase is encoded by the endogenous Kosakonia gene and wherein the endogenous Kosakonia gene is operably linked to the promoter which is repressed by the TetR repressor protein.

12. The bacterium of claim 1, wherein:
(i) the first recombinant DNA molecule comprises the Klebsiella variicola pstS phosphate-sensitive promoter having at least 95% sequence identity to SEQ ID NO: 515, the BCD22vL ribosome binding site (RBS) of SEQ ID NO: 449, and the transcriptional repressor protein comprising the amino acid sequence having at least 95% sequence identity to SEQ ID NO: 516; and
(ii) the second recombinant DNA molecule comprises the Ptet promoter having at least 95% sequence identity to SEQ ID NO: 32, and the bacterial glutamine synthetase is a glutamine synthetase having at least 95% sequence identity to SEQ ID NO: 229.

13. A composition comprising the genetically engineered bacterium of claim 1 and an agriculturally acceptable carrier.

14. The composition of claim 13, wherein the composition further comprises:
(i) an agriculturally acceptable adjuvant, optionally wherein the adjuvant comprises an adhesive agent, a desiccant, and/or a dispersant;
(ii) a fungicide, an insecticide, a nematicide, a rodenticide, and/or a bacteriocide; and/or
(iii) a fertilizer, optionally wherein the fertilizer comprises nitrogen, phosphorous, potassium, calcium, sulfur, magnesium, boron, chloride, manganese, iron, zinc, copper, molybdenum, and/or selenium.

15. A plant part or plant propagule which is at least partially coated, imbibed, or mixed with the composition of claim 13.

16. A method of providing nitrogen to a plant comprising placing the one genetically engineered bacterium of claim 1 into a plant growth medium, wherein a decrease in phosphate concentration in the plant growth medium results in the production of ammonia which is provided to the plant when grown in the growth medium.

17. The method of claim 16, wherein the plant growth medium comprises soil and/or water, optionally wherein the soil and/or water is non-axenic.

18. The method of claim 16, wherein the placing is prior to, during, and/or after depositing a seed or vegetative propagule in the plant growth medium.

19. The method of claim 16, wherein the plant growth medium has been treated by adding inorganic phosphate to the plant growth medium at a rate of about 3 g/m² to about 15 g/m².

20. The method of claim 16, further comprising;
(i) determining leaf nitrogen and/or chlorophyll concentrations in a plant grown in the plant growth medium; and
(ii) placing or re-applying the genetically engineered bacterium into the plant growth medium and/or in contact with the plant when the leaf nitrogen and/or chlorophyll concentrations in the plant are sub-optimal for yield.

21. An agricultural system comprising:
(i) at least one engineered bacterium of claim 1;
(ii) at least one plant growth medium; and
(iii) at least one crop plant, crop plant seed, or crop plant vegetative propagule; wherein the plant growth medium, crop plant, crop seed, and/or crop plant propagule comprise, are at least partially coated, imbibed, and/or are mixed with the engineered bacterium or a composition comprising the engineered bacterium and an agriculturally acceptable carrier.

22. The system of claim 21, wherein the crop plant, seed, or vegetative propagule is an alfalfa, apple, banana, barley, bean, buckwheat, cabbage, cassava, chili, clover, coffee, corn, cotton, cowpea, cucumber, fonio, garlic, herb, lettuce, maize, melon, millet, nut, oat, oilseed rape, olive, onion, orange, sunflower, pea, Phaseolus bean, plantain, potato, quinoa, rice, rye, safflower, sorghum, soybean, sugar beet, sugar cane, sunflower, tangerine, tobacco, tomato, triticale, turnip, wheat, or yam plant, seed, or vegetative propagule.

23. The system of claim 21, wherein the plant growth medium comprises soil and/or water, optionally wherein the soil and/or water is non-axenic.

24. The system of claim 21, wherein the vegetative propagule comprises a cutting, tuber, or stolon.

25. A treated plant seed or plant propagule system comprising:
(i) at least one crop plant seed or crop plant vegetative propagule; and
(ii) at least one engineered bacterium of claim 1,
wherein the crop plant seed or crop plant propagule are at least partially coated, imbibed, and/or mixed with the engineered bacterium or a composition comprising the engineered bacterium and an agriculturally acceptable carrier.

26. The system of claim 25, wherein the crop plant, seed, or vegetative propagule is an alfalfa, apple, banana, barley, bean, buckwheat, cabbage, cassava, chili, clover, coffee, corn, cotton, cowpea, cucumber, fonio, garlic, herb, lettuce, maize, melon, millet, nut, oat, oilseed rape, olive, onion, orange, sunflower, pea, Phaseolus bean, plantain, potato, quinoa, rice, rye, safflower, sorghum, soybean, sugar beet, sugar cane, sunflower, tangerine, tobacco, tomato, triticale, turnip, wheat, or yam plant, seed, or vegetative propagule.
